(19) United States
(12) Patent Application Publication
(10) Pub. No.: US 2010/0252433 A1
(43) Pub. Date: Oct. 7, 2010

(54) NOVEL OPTICAL LABELING MOLECULES FOR PROTEOMICS AND OTHER BIOLOGICAL ANALYSES

(76) Inventors: Edward DRATZ, Bozeman, MT (US); Paul Grieco, Bozeman, MT (US)

Correspondence Address: COOLEY LLP;ATTN: Patent Group, Suite 1100, 777 - 6th Street, NW, WASHINGTON, DC, 20001, US

(21) Appl. No.: 12/578,419

(22) Filed: Oct. 13, 2009

Related U.S. Application Data
(63) Continuation-in-part of application No. PCT/US08/59963, filed on Apr. 10, 2008.
(60) Provisional application No. 60/911,051, filed on Apr. 10, 2007.
(60) Provisional application No. 60/916,548, filed on May 7, 2007.
(60) Provisional application No. 61/245,516, filed on Sep. 24, 2009.
(60) Provisional application No. 61/245,527, filed on Sep. 24, 2009.
(60) Provisional application No. 61/245,537, filed on Sep. 24, 2009.
(60) Provisional application No. 61/245,548, filed on Sep. 24, 2009.
(60) Provisional application No. 61/245,552, filed on Sep. 24, 2009.

(51) Int. Cl. G01N 33/68; C07D 207/08; C07D 403/06; C07F 5/02; C07F 17/00; G01N 33/573

(52) U.S. Cl. 204/451; 548/537; 548/524; 548/405; 546/4; 436/86; 435/4; 204/456; 204/461

(57) ABSTRACT

The invention relates to compositions and methods useful in the labeling and identification of changes in protein levels, changes in enzyme activity, and changes in protein modification. The invention provides for highly soluble optical labeling molecules which are optionally cleavable after separation of mixtures of labeled proteins into components. These optical labeling molecules find utility in a variety of applications, including use in the field of proteomics.

### CROSS-REFERENCE TO RELATED APPLICATIONS

The present application is a continuation-in-part application under 37 C.F.R. §1.53(b) of pending International Patent Application No. PCT/US2008/059963 filed Apr. 10, 2008, which claims priority to U.S. Provisional Application No. 60/911,051, filed Apr. 10, 2007 and U.S. Provisional Application No. 60/916,548, filed May 7, 2007; and also claims priority to U.S. Provisional Application No. 61/245,516 filed Sep. 24, 2009; U.S. Provisional Application No. 61/245,427, filed Sep. 24, 2009; U.S. Provisional Application No. 61/245,537, filed Sep. 24, 2009; U.S. Provisional Application No. 61/245,548, filed Sep. 24, 2009 and U.S. Provisional Application No. 61/245,552, filed Sep. 24, 2009. This application also claims priority to U.S. patent application Ser. No. 12/551,114, filed Aug. 31, 2009, which is a Continuation of U.S. patent application Ser. No. 10/761,818, filed Jan. 20, 2004, now U.S. Pat. No. 7,585,260, which is Continuation-in-Part of U.S. patent application Ser. No. 10/623,447, filed Jul. 18, 2003, now abandoned. This application also claims priority to U.S. patent application Ser. No. 11/767,404, filed Jun. 22, 2007 and U.S. patent application Ser. No. 11/767,406, filed Jun. 22, 2007 both of which are a Divisional of U.S. patent application Ser. No. 10/761,818 filed Jan. 20, 2004, now U.S. Pat. No. 7,585,260. This Application also claims priority to International Application No. PCT/US2003/022397 filed Jul. 18, 2003; all of which claims the benefit of the priority date of U.S. Provisional Application No. 60/396,950, filed Jul. 18, 2002, the disclosures of which are herein incorporated by reference in its entirety for all purposes.

### GOVERNMENT INTERESTS

This research was supported by the US National Science Foundation Grant MCB 0139957, the US National Institutes of Health Grants R21RR16240 and R41RR021790, and the Montana Board of Research and Commercializaton of Technology grants #05-14, #06-46, and #07-17.

### FIELD

The invention relates to compositions and methods useful in the labeling and identification of changes in levels of proteins, changes in enzyme activities, and changes in protein modifications. The invention provides for highly soluble optical labeling molecules which are optionally cleavable after separation of mixtures of labeled proteins into components. These optical labeling molecules find utility in a variety of applications, including use in the field of proteomics.

### BACKGROUND

Proteomics is the practice of identifying and quantifying the proteins, or the ratios of the amounts of proteins expressed in cells and tissues and their posttranslational modifications under different physiological conditions. Proteomics provides methods of studying the effect of biologically relevant variables on gene protein production and modification that provides advantages over genomic studies. While facile DNA gene microarray methods have been rapidly developed and are widely available for analysis of mRNA levels, recent studies have shown little correlation between mRNA levels and levels of protein expression (Gygi et al., (1999) Mol. Cell. Biol. 19, 1720-1730; Anderson et al., (1997) Electrophoresis 18: 533-537; Feder and Waser (2005)., J Evol Biol 18(4): 901-10).

A major limitation of current proteomics techniques is the lack of compositions and methods of sufficient sensitivity to detect low levels of intact proteins and the relative amounts of these low levels of proteins. For example, intact proteins present at low copy number are difficult to detect using currently available methods that generally rely on the use of covalent dyes to label proteins and peptides.

In general, dyes currently used in the art for protein detection during proteomic analysis possess a number of undesirable qualities. Notably, covalently attaching a dye to a protein before separation often results in a substantial decrease in protein solubility which often leads to loss of detectable proteins. With currently available dye molecules that are useful for detection on gels, protein solubility decreases as the number of dye molecules attached per protein molecule increases. Thus, the lack of dye sensitivity cannot be countered by adding more dye molecules to the protein. Methods that rely on detecting proteins with dyes or other stains after separation suffer from lack of sensitivity, do not allow multicolor, multiplex detection, and may have low dynamic range for detection, such as when using silver staining.

Thus, a need exists for optical labeling molecules that possess increased sensitivity and water solubility which enhances detection sensitivity and recovery of intact proteins and allows for versatile multiplex analysis of intact proteins for proteomics. Accordingly, intact proteins of interest that show changes in amount or changes in enzyme activity can be more effectively selected and isolated for analysis of protein identity and posttranslational protein modifications. In addition, there is a need for high sensitivity optical labeling molecules which can be removed after separation and before identification and analysis by mass spectral methods.

### SUMMARY

In one embodiment, the present invention provides an optical labeling molecule selected from the group consisting of structural Formula (I), structural Formula (III), structural Formula (XXI), structural Formula (XV), and structural formula (XV′), or a salt or solvate thereof,

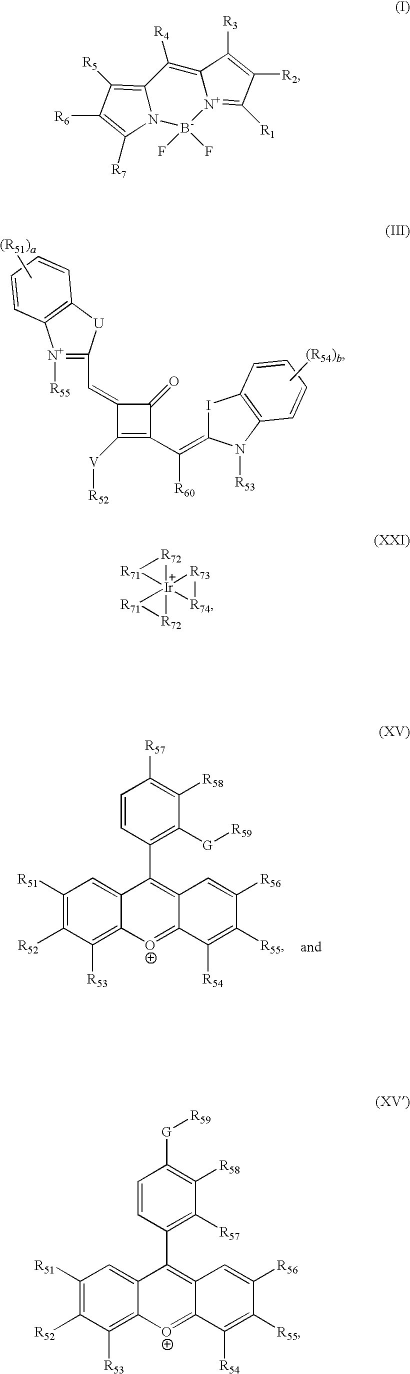

wherein the optical labeling molecule comprises a fluorophore with a derivative tail, and the derivative tail comprises at least one amide bond, wherein:
R₁ to R₇, and R₅₁ to R₅₉, are each independently hydrogen, acyl, substituted acyl, alkoxy, substituted alkoxy, alkoxycarbonyl, substituted alkoxycarbonyl, alkyl, substituted alkyl, amino, substituted amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —(CH₂)ₙN⁺(CH₃)₃, —S(O)ₜR₂₀, —SO₃H, —(CH₂)ₙS(O)ₙOH, —(CH₂)ₙS(O)₂O⁻, —OP(O)(O⁻)₂, or —(CH₂)ₙOP(O)(O⁻)₂,

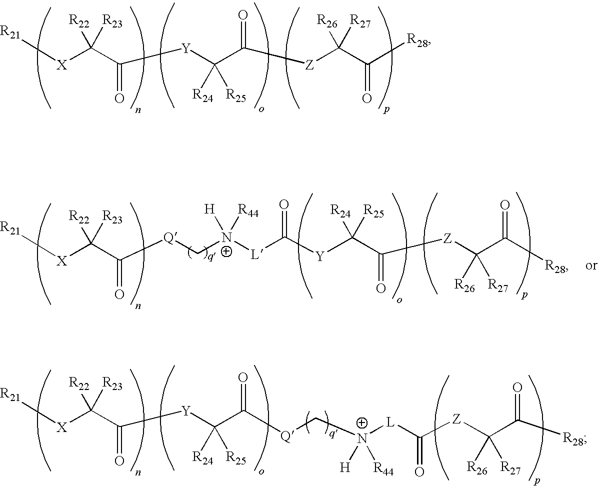

provided that one and only one of R₁ to R₇, or one and only one of R₅₂ to R₅₅, or one and only one of R₅₇ to R₅₉ is

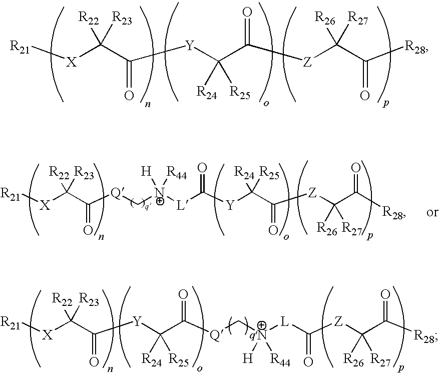

R₇₁ to R₇₄ are each independently aryl, substituted aryl, heteroaryl, or substituted heteroaryl, provided that R₇₁ to R₇₄ contain at least one amino, substituted amino, acyl, substituted acyl,

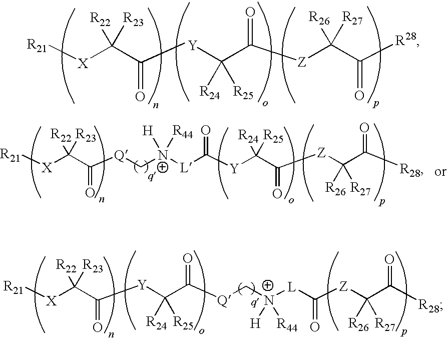

R₂₂, R₂₃, R₂₄, R₂₅, R₂₆ and R₂₇ are each independently hydrogen, alkyl, substituted alkyl, amino, substituted amino, aryl, substituted aryl, arylalkyl, substituted arylalkyl, heteroaryl, substituted heteroaryl, heteroarylalkyl, heteroalkyl or substituted heteroalkyl, nitro, —(CH₂)ₙN⁺(CH₃)₃, —S(O)ₜR₂₀, —SO₃H, —(CH₂)ₙS(O)OH, —(CH₂)ₙS(O)₂O⁻, —OP(O)(O⁻)₂, or —(CH₂)ₙOP(O)(O⁻)₂;
R₂₁ is —(CH₂)ₘ—C(O)—, —(CH₂)ₘ—C(O)-Q′(CH₂)_{q}—N⁺H(R₄₆)-L′-C(O)—,

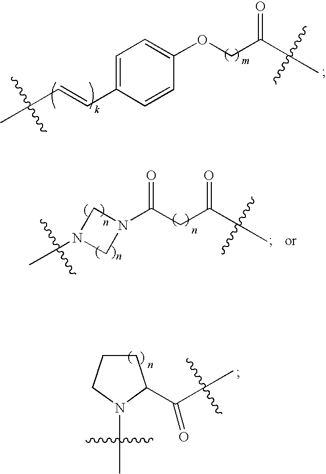

R₂₈ is -Q-L-C(O)-A; -Q(CH₂)_{q}—N⁺H(R₄₅)-L-C(O)-A, -Q-L-D-C(O)—(B′)ᵣ-A or -Q(CH₂)_{q}—N⁺H(R₄₅)-L-D-C(O)—(B′)ᵣ-A;
R₂₀ and R₄₃ are independently alkyl, substituted alkyl, aryl, substituted aryl, arylalkyl, substituted arylalkyl, heteroaryl, substituted heteroaryl, heteroarylalkyl, heteroalkyl or substituted heteroalkyl;
R₂₉ to R₃₄ and R₄₄ to R₄₇ are independently hydrogen, alkyl, or substituted alkyl;
a and b are independently 0, 1, 2, 3 or 4;
k and m are independently 1, 2, 3, 4 or 5;
h, n, o and p are independently 0, 1, 2, 3, 4 or 5;
q and q′ are independently 2, 3, 4 or 5;
e and t are independently 0, 1 or 2;
Q is —NR₂₉;
X is —NR₃₀ or —O—;
Y is —NR₃₁ or —O—;
Z is —NR₃₂ or —O—;
Q′ is —NR₃₃;
B′ is —NH—C(R₃₄)—C(O)— wherein R₃₄ is hydrogen, alkyl, substituted alkyl, aryl, substituted aryl, arylalkyl, substituted arylalkyl, heteroaryl, substituted heteroaryl, heteroarylalkyl, heteroalkyl or substituted heteroalkyl;
I is —C(R₅₆R₅₇)—, —S—, —O— or —Se—;
U is —C(R₅₈R₅₉)—, —S—, —O— or —Se—;
R₆₀ is hydrogen or alternatively R₆₀ and R₅₃ together with the atoms to which they are bonded form a cycloheteroalkyl or substituted cycloheteroalkyl ring;
R₆₁ is hydrogen or alternatively R₆₀ and R₅₂ together with the atoms to which they are bonded form a cycloheteroalkyl or substituted cycloheteroalkyl ring;
V is —NR₆₁,

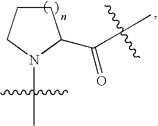

or —O—;
r is 0 or 1;
L and L′ are alkyl, substituted alkyl, heteroalkyl or substituted alkyl, aryl or substituted aryl;
A is OH, —NHCH₂CH₂SH,

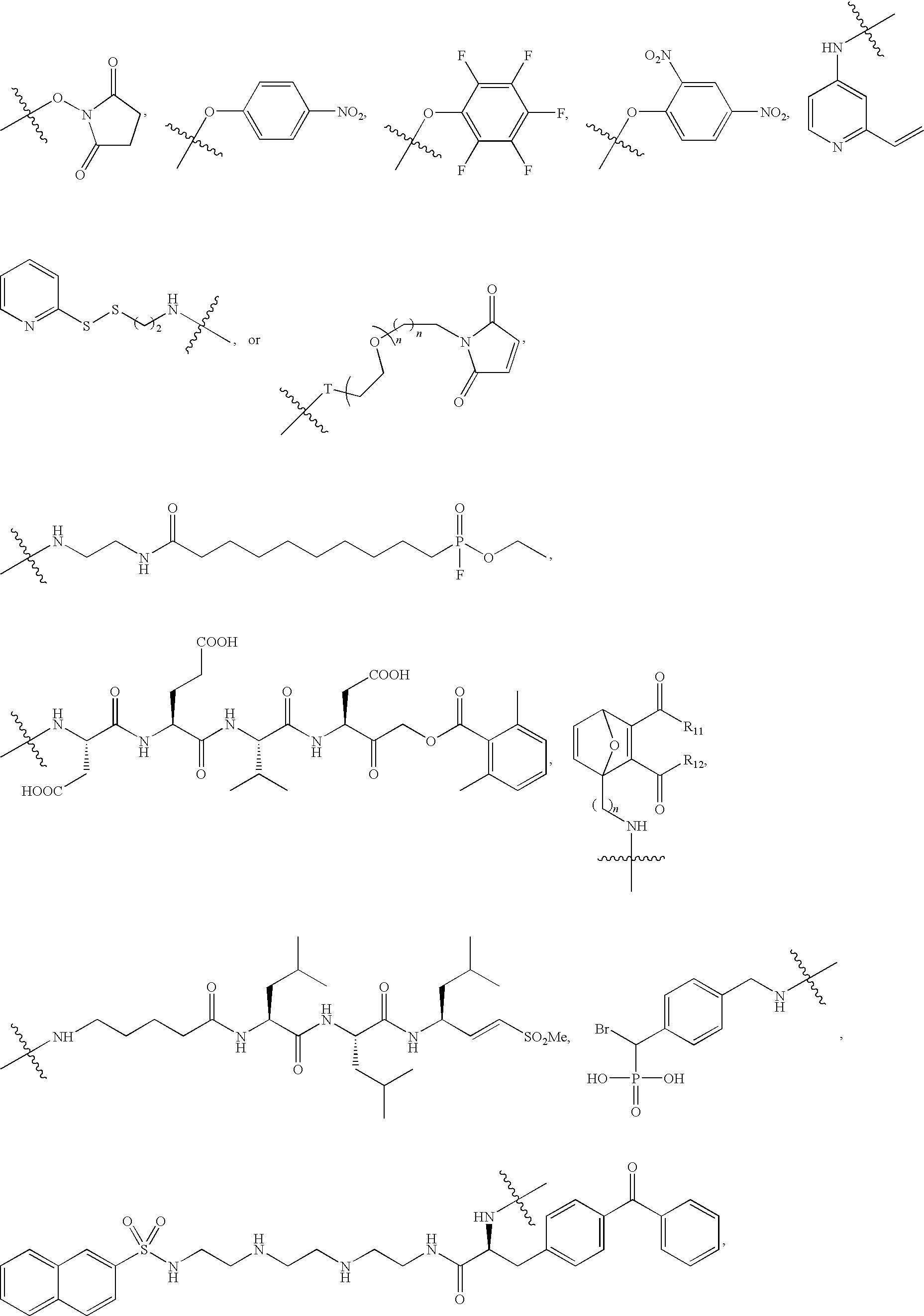

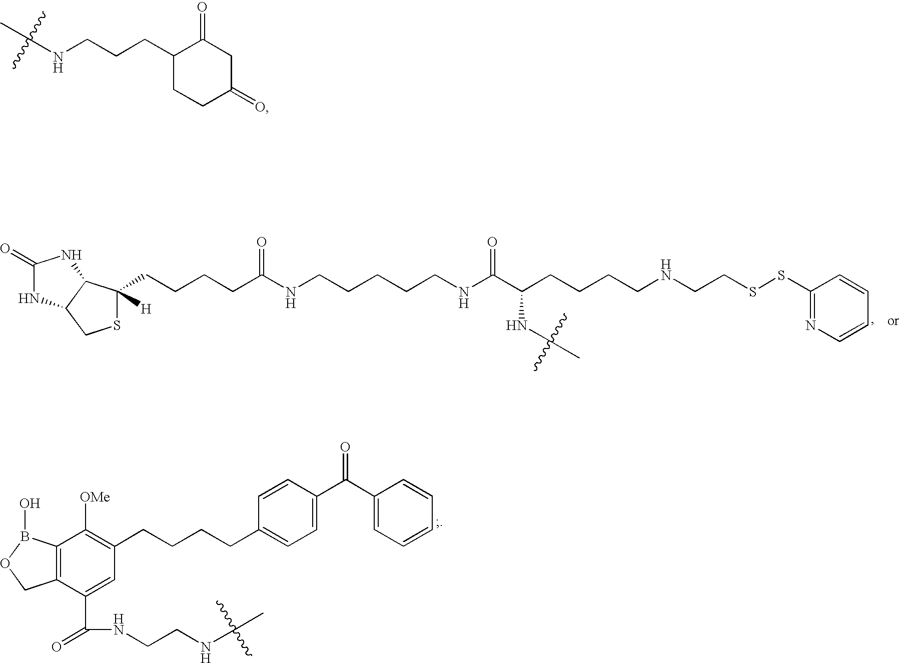

R₁₁ and R₁₂ are independently alkyl, substituted alkyl, acyl, substituted acyl, alkoxy, substituted alkoxy, aryl, substituted aryl, azido alkyl, alkynyl, substituted alkynyl, amino, or substituted amino;
T is —NR₃₄;
D is

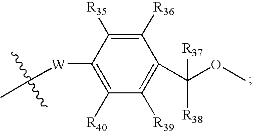

R^{c} is H, alkyl or can be taken together with the nitrogen atoms to which they are bonded form a cycloheteroalkyl or substituted cycloheteroalkyl ring;
R₃₇ and R₃₈ are independently hydrogen, alkyl or substituted alkyl;
R₃₅, R₃₆, R₃₉ and R₄₀ are independently hydrogen, nitro, alkyl, substituted alkyl, —NR₄₁R₄₂, —S(O)_{c}R₄₃, aryloxy, substituted aryloxy, alkoxy or substituted alkoxy provided that at least one of R₃₅, R₃₆, R₃₇ and R₃₈ is nitro, aryloxy, substituted aryloxy, alkoxy or substituted alkoxy; and
W is —O—, —S— or —NR₄₇;
provided that the optical labeling molecule contains at least one zwitterionic pair.

In another embodiment, the present invention provides a method of differential analysis of proteins comprising:

providing at least two different samples of differently labeled proteins with at least two different optical labeling molecules to form pluralities of differently labeled proteins, wherein said optical labeling molecule comprises a zwitterionic dye moiety, a linker, an optional titratable group that mimics the acid-base titration of the group labeled in the proteins, an optional cleavable group, an optional second label stable isotope group and an activator that covalently attaches the optical labeling molecule to the protein,

mixing the different samples of differently labeled proteins together to form a mixture;

simultaneously separating the differently labeled proteins in the mixture to obtain a plurality of separated differently labeled proteins;

scanning the separated differently labeled proteins;

matching the same proteins from different samples that have been labeled with the different optical labeling molecules; and

simultaneously determining the changes in relative amounts of differently labeled proteins in the different samples by correlating said changes with the strength of the optical images of the labeled proteins.

In another embodiment, the present invention provides a method of differential analysis of proteins comprising:

covalently labeling at least two different samples of proteins with at least two different optical labeling molecules to form pluralities of differently labeled proteins, wherein said optical labeling molecule as described above;

mixing the different samples of differently labeled proteins together to form a mixture;

simultaneously separating the differently labeled proteins in the mixture to obtain a plurality of separated differently labeled proteins;

scanning the separated differently labeled proteins;

matching the same proteins from different samples that have been labeled with the different optical labeling molecules; and

simultaneously determining the changes in relative amounts of differently labeled proteins in the different samples by correlating said changes with the strength of the optical images of the labeled proteins.

### BRIEF DESCRIPTION OF THE DRAWINGS

FIG. 1 is a representative 2D gel image of cytosolic brain fraction shown in black and white images limited to 8 bit resolution, which equals 256 image levels. First dimension—18 cm, pH 3-10 isoelectric focusing. 2^{nd} dimension—11% non-gradient gel, SDS-PAGE. The circled spots—are ranked by signification of differential protein expression between the two samples.

FIG. 2 is the MS and MS/MS data of one of the three detected peptides that led to GSTO1 identification.

### DETAILED DESCRIPTION

### Definitions

All documents cited in the present specification are incorporated by reference in their entirety for all purposes.

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In some embodiments, an alkyl group comprises from 1 to 20 carbon atoms (C₁-C₂₀ alkyl). In other embodiments, an alkyl group comprises from 1 to 10 carbon atoms (C₁-C₁₀ alkyl). In still other embodiments, an alkyl group comprises from 1 to 6 carbon atoms (C₁-C₆ alkyl).

“Alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl,” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkoxy,” by itself or as part of another substituent, refers to a radical of the formula —O—R₁₀₀, where R₁₀₀ is alkyl or substituted alkyl as defined herein.

“Alkoxycarbonyl,” by itself or as part of another substituent, refers to a radical of the formula —C(O)—R₁₀₀, where R₁₀₀ is as defined above.

“Acyl” by itself or as part of another substituent refers to a radical —C(O)R₁₀₁, where R₁₀₁ is hydrogen, alkyl, substituted alkyl, aryl, substituted aryl, arylalkyl, substituted arylalkyl, heteroalkyl, substituted heteroalkyl, heteroarylalkyl or substituted heteroarylalkyl as defined herein. Representative examples include, but are not limited to formyl, acetyl, cyclohexylcarbonyl, cyclohexylmethylcarbonyl, benzoyl, benzylcarbonyl and the like.

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryl group comprises from 6 to 20 carbon atoms (C₆-C₂₀ aryl). In other embodiments, an aryl group comprises from 6 to 15 carbon atoms (C₆-C₁₅ aryl). In still other embodiments, an aryl group comprises from 6 to 15 carbon atoms (C₆-C₁₀ aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In some embodiments, an arylalkyl group is (C₆-C₃₀) arylalkyl, e.g., the alkanyl, alkenyl or alkynyl moiety of the arylalkyl group is (C₁-C₁₀) alkyl and the aryl moiety is (C₆-C₂₀) aryl. In other embodiments, an arylalkyl group is (C₆-C₂₀) arylalkyl, e.g., the alkanyl, alkenyl or alkynyl moiety of the arylalkyl group is (C₁-C₈) alkyl and the aryl moiety is (C₆-C₁₂) aryl. In still other embodiments, an arylalkyl group is (C₆-C₁₅) arylalkyl, e.g., the alkanyl, alkenyl or alkynyl moiety of the arylalkyl group is (C₁-C₅) alkyl and the aryl moiety is (C₆-C₁₀) aryl.

“Aryloxycarbonyl,” by itself or as part of another substituent, refers to a radical of the formula —C(O)—O—R₁₀₂, where R₁₀₂ is aryl, substituted aryl, arylalkyl, substituted arylalkyl, heteroaryl, substituted heteroaryl, heteroarylalkyl or substituted heteroarylalkyl.

“Cycloalkyl,” by itself or as part of another substituent, refers to a saturated or unsaturated cyclic alkyl radical, as defined herein. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In some embodiments, the cycloalkyl group comprises from 3 to 10 ring atoms (C₃-C₁₁) cycloalkyl). In other embodiments, the cycloalkyl group comprises from 3 to 7 ring atoms (C₃-C₇ cycloalkyl).

“Cycloalkylalkyl,” by itself or as part of another substituent, refers to an acyclic alkyl group in which one of the hydrogen atoms bonded to a carbon atom, typically a terminal or sp³ carbon atom, is replaced with an cycloalkyl group as, as defined herein.

“Cycloheteroalkyl,” by itself or as part of another substituent, refers to a saturated or unsaturated cyclic alkyl radical in which one or more carbon atoms (and optionally any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidone, quinuclidine, and the like. In some embodiments, the cycloheteroalkyl group comprises from 3 to 10 ring atoms (3-10 membered cycloheteroalkyl) In other embodiments, the cycloalkyl group comprise from 5 to 7 ring atoms (5-7 membered cycloheteroalkyl). A cycloheteroalkyl group may be substituted at a heteroatom, for example, a nitrogen atom, with a (C₁-C₆) alkyl group. As specific examples, N-methyl-imidazolidinyl, N-methyl-morpholinyl, N-methyl-piperazinyl, N-methyl-piperidinyl, N-methyl-pyrazolidinyl and N-methyl-pyrrolidinyl are included within the definition of “cycloheteroalkyl.” A cycloheteroalkyl group may be attached to the remainder of the molecule via a ring carbon atom or a ring heteroatom.

“Cycloheteroalkylalkyl,” by itself or as part of another substituent, refers to an acyclic alkyl group in which one of the hydrogen atoms bonded to a carbon atom, typically a terminal or sp³ carbon atom, is replaced with an cycloheteroalkyl group as, as defined herein.

“Heteroalkyl,” “Heteroalkanyl,” “Heteroalkenyl” and “Heteroalkynyl,” “by themselves or as part of other substituents, refer to alkyl, alkanyl, alkenyl and alkynyl groups, respectively, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, O, S, N, Si, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR₁₀₃R₁₀₄—, —N═N—, —N═N—NR₁₀₅R₁₀₆, —PR₁₀₇—, —P(O)₂—, —POR₁₀₅—, —O—P(O)₂—, —SO—, —SO₂—, —SnR₁₀₀R₁₁₀— and the like, where R₁₀₃-R₁₀₈ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl,” by itself or as part of another substituent, refers to a monovalent heteroaromatic radical derived by the removal of one hydrogen atom from a single atom of a parent heteroaromatic ring systems, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group comprises from 5 to 20 ring atoms (5-20 membered heteroaryl). In other embodiments, the heteroaryl group comprises from 5 to 10 ring atoms (5-10 membered heteroaryl). Exemplary heteroaryl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl,” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In some embodiments, the heteroarylalkyl group is a 6-21 membered heteroarylalkyl, e.g., the alkanyl, alkenyl or alkynyl moiety of the heteroarylalkyl is (C₁-C₆) alkyl and the heteroaryl moiety is a 5-15-membered heteroaryl. In other embodiments, the heteroarylalkyl is a 6-13 membered heteroarylalkyl, e.g., the alkanyl, alkenyl or alkynyl moiety is (C₁-C₃) alkyl and the heteroaryl moiety is a 5-10 membered heteroaryl.

“Optical labeling molecule,” by itself or as part of another substituent refers to any molecule useful in covalently labeling biological molecules that permits the labeled molecule to be detected with an optical measurement and includes any dye molecule disclosed herein such as those encompassed by structural Formulae (I)-(XXI). Optical measurements include, but are not limited to color, absorbance, luminescence, fluorescence, phosphorescence, with fluorescence usually being preferred for maximum detection sensitivity. Optical labeling molecules may be identified either by their chemical structure and/or chemical name. When the chemical structure and chemical name conflict, the chemical structure is determinative of the identity of the optical labeling molecules. The optical labeling molecules described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated optical labeling molecules including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The optical labeling molecules may also exist in several tautomeric forms including the enol form, the keto form and mixtures thereof. Accordingly, the chemical structures depicted herein encompass all possible tautomeric forms of the illustrated optical labeling molecules. The optical labeling molecules described herein also include isotopically labeled optical labeling molecules where one or more atoms have an atomic mass different from the atomic mass conventionally found in nature. Examples of isotopes that may be incorporated into the optical labeling molecules include, but are not limited to, ²H, ³H, ¹³C, ¹⁴C, ¹⁵N, ¹⁸O, ¹⁷O, etc. Optical labeling molecules may exist in unsolvated forms as well as solvated forms, including hydrated forms and as N-oxides. In general, optical labeling molecules may be hydrated, solvated or N-oxides. Certain optical labeling molecules may exist in multiple crystalline or amorphous forms. In general, all physical forms are equivalent for the uses contemplated herein and are intended to be within the scope of the present invention. Further, it should be understood, when partial structures of the compounds are illustrated, that brackets or wiggled lines indicate the point of attachment of the partial structure to the rest of the molecule.

“Parent Aromatic Ring System,” refers to an unsaturated cyclic or polycyclic ring system having a conjugated it electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System,” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Protecting group,” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Salt,” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, trifluoroacetic acid and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —(O)P(O)(O⁻)₂, —(O)P(O)(OR^{b})(O), —(O)P(O)(OR^{b})(OR^{b}), —C(O)R^{b}, —C(s)R^{b}, —C(NR)R^{b}, —C(O)OR^{b}, —C(s)OR^{b}, —(O)P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)OR^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s are taken together with the nitrogen atom to which they are bonded form a 4-, 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl.

Similarly, substituent groups useful for substituting unsaturated carbon atoms in the specified group or radical include, but are not limited to, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —(O)P(O)(O⁻)₂, —(O)P(O)(OR^{b})(O⁻), —(O)P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined. Substituent groups useful for substituting nitrogen atoms in heteroalkyl and cycloheteroalkyl groups include, but are not limited to, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂O—OS(O)₂R^{b}, —OS(O)₂O⁻, —OS(O)₂OR^{b}, —(O)P(O)(O⁻)₂, —(O)P(O)(OR^{b})(O), —(O)P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined. Substituent groups from the above lists useful for substituting other specified groups or atoms will be apparent to those of skill in the art. The substituents used to substitute a specified group can be further substituted, typically with one or more of the same or different groups selected from the various groups specified above.

### Optical Labeling Molecules

The present invention is directed toward compositions and methods useful in optical labeling and detection of biomolecules such as proteins. One aspect of the invention encompasses the use of optical labeling molecules in the field of proteomics. A significant problem with existing methods is limited detection sensitivity. Currently available dyes, suffer from several shortcomings which include, for example, reducing the solubility of proteins to which they are attached. For example, some prior art dyes require a very low multiplicity of dye labeling (1% to 3% dyes/protein,) to minimize dye-induced reduction in protein solubility and dye-induced mobility shifts which severely limits the sensitivity attainable.

Accordingly, optical labeling molecules described herein have increased aqueous solubility over a wide pH range and enhanced detection sensitivity. The optical labeling molecules described herein typically contain zwitterionic groups which maintain charge over a wide pH range and thus increase the solubility of labeled proteins in both aqueous and mixed polar solvents while minimizing isoelectric point (pI) shifts, which facilitates separation and identification of the labeled proteins.

In general, an optical labeling molecule is detected through measuring fluorescent emission. In some embodiments, the optical labeling moiety is a fluorescent dye. Suitable fluorophores include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”), pyrene, Malacite green, stilbene, Lucifer Yellow, Cascade Blue®, Texas Red, Cy dyes (Cy2, Cy3, Cy5, Cy5.5, Cy7, etc.), Alexa dyes (including, but not limited to, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700 and Alexa Fluor 750, see Molecular Probes catalog, 9th Edition), phycoerythin, BODIPY dyes and derivatives, and others described in the 9th Edition of the Molecular Probes Handbook by Richard P. Haugland and in U.S. Pat. Nos. 6,130,101, 6,162,931 and 6,291,203.

In some embodiments, the optical labeling molecule includes a zwitterionic dye moiety, a linker, a titratable group to replace the acid-base behavior of the target group on proteins used for coupling and an activator. In other embodiments, the zwitterionic dye moiety includes more than one zwitterionic group to further enhance the solubility of zwitterionic dyes and zwitterionic dye-labeled proteins over a wide pH range. Zwitterionic groups are those that contain both positive and negative charges and are net neutral, but highly charged. By “zwitterionic dye moiety” is meant a dye that is designed to contain one or more zwitterionic charge pairs, generally added as “zwitterionic components”, e.g., separate positive and negative charged groups. In some embodiments, the zwitterionic dye moiety is non-titratable and thus maintains its zwitterionic charge character over a wide pH range (e.g., pH 3-pH 12, pH 4-pH 10, pH 5-pH 9 and pH 6-pH 11).

In other embodiments, the dye moiety, for example, a fluorophore, is derivatized to include side chain groups and/or a “tail” for the addition of some or all the components of zwitterionic charge pairs. Any number of dyes can be derivatized to allow for a zwitterionic charge balance and other appropriate components (e.g., titratable groups, isotopes, activators, cleavable groups, etc.).

In one embodiment, the derivative tail contains at least one amide bond. In other embodiments, the derivative tail contains at least two amide bonds.

In general, charged groups are contained in the dye moiety. In general, pairs of positive and negative charged moieties (“the zwitterionic components”) may be added at separate locations to the dye moiety, although in some embodiments, both the positive and negative charges are added as single “branched” moieties or combinations thereof. In other embodiments, the chromophoric framework of the dye includes positively or negatively charged groups or includes some combination of positive and negative charges and suitable charged groups to make the number of positive and negative groups equal (in order to form one or more zwitterionic pairs). In still other embodiments, the fluorophore has a derivative “tail,” used as a linker to the other components of the optical labeling moiety, which can contain zwitterionic components as well. In still additional embodiments, the zwitterionic components are anywhere within the optical labeling moiety. For example, negative charges can be added to the fluorophore and positive charges to the linker moiety, or vice-versa.

In some embodiments, the zwitterionic components are small alkyl groups (C₂-C₄) with quaternary ammonium groups (—NR₄⁺), guanidinium groups or other positively charged groups which are not titratable until about pH 12 and negatively charged alkyl sulfonate or alkyl sulfate groups. Any other charged groups which are not titratable between pH 3-12 and are stable under aqueous conditions may be included as components of zwitterionic groups.

In some embodiments, the optical labeling moiety is a BODIPY dye of structural formula (I), wherein R₁-R₇ includes at least one zwitterionic component. In other embodiments, the optical labeling moiety is a BODIPY dye of structural formula (I) where the R₁ position includes a derivative “tail” that may include a number of different chemical groups, the R₃R₅, and R₇ positions can be used to add zwitterionic components and the R₃, R₄, R₅ and R₇ positions may be used to create other BODIPY type dyes with different colors. In still other embodiments, the compounds of structural formula (I) are substituted with one, two or more quaternary ammonium groups and one, two or more sulfonate groups.

BODIPY dyes with a narrow excitation spectra and a wide range of excitation/emission spectra are readily available (9th Edition of the Molecular Probes Handbook). BODIPY dyes have similar structures but different excitation and emission spectra that allow multiplex detection of proteins from two or more protein sample mixtures simultaneously on the same gel. Multiplex detection, or multiplexing, is defined as the transmission of two or more messages simultaneously with subsequent separation of the signals at the receiver.

In some embodiments, a double zwitterionic substitution of two quaternary ammonium and two sulfonate groups are added to a neutral dye moiety. In other embodiments, the double zwitterionic substitution of two quaternary ammonium and sulfonate groups are added to a BODIPY dye moiety.

General methods for designing useful optical labeling molecules can be described. A first method is exemplified by Cascade Blue or Alexa dyes where the dye structure is relatively polar and compact, but there is a net charge on the dye that would substantially alter the isoelectric points of labeled proteins. A tail is designed which may include nontitratable opposing charges to form nontitratable zwitterionic charge pairs, additional zwitterionic charge pairs, titratable groups to replace the acid/base properties of protein groups that are modified by the activator, an optional cleavable group, an optional second label stable isotope group and an activator.

A second general method for designing dyes is exemplified by the BODIPY scaffold where dye components are designed, synthesized and assembled to provide the desired dye properties. Briefly, a tail or dye chromophore may be designed which include nontitratable opposing charges to form nontitratable zwitterionic charge pairs, additional zwitterionic charge pairs, titratable groups to replace the acid/base properties of protein groups that are modified by the activator, an optional cleavable group, an optional second label stable isotope group and an activator.

In some embodiments, in addition to the dye moiety, the optical labeling molecule further includes a linker, an optional titratable group and an activator. By “titratable group” is meant a group that mimics the acid-base titration of the group labeled on the target molecule. The charge on the group labeled on the target molecule is often lost when the target molecule forms a covalent bond with the activator of the optical labeling molecule. In some embodiments, the titratable group is present in the optical labeling molecule and the titratable group replaces the lost charge and thus maintains, as closely as possible, the isoelectric points of the labeled target molecule. In some embodiments, the target molecule is a protein. In these embodiments, the titratable group replaces the charge lost when the activator forms a covalent bond with the protein, thus maintaining the isoelectric point of the protein which is an important factor in protein separation using techniques, such as, for example, two-dimensional electrophoresis, ion exchange chromatography, capillary electrophoresis and reverse phase chromatography.

In other embodiments, in addition to a dye moiety, a linker and an optional titratable group moiety, the optical labeling molecule further includes an activator. The activator covalently attaches an optical labeling molecule to the target molecule. Other activators include, but are not limited to, succinimidyl groups, sulfosuccinimidyl groups, imido esters, isothiocyanates, aldehydes, sulfonylchlorides, arylating agents, thiols, maleimides, iodoacetamides, alkyl bromides, vinyl pyridines, pyridine disulfides, methyl methanethiosulphonate and benzoxidiazoles.

The activator forms a covalent bond with one or more sites on a target protein. By “protein” or grammatical equivalents herein is meant proteins, oligopeptides and peptides, derivatives and analogs, including proteins containing non-naturally occurring amino acids, amino acid analogs and peptidomimetic structures.

In some embodiments, the type and number of proteins labeled is determined by the method used or desired result. In some instances, most or all of the proteins of a cell or virus are labeled. In other instances, some subsets are labeled. For example, subcellular fractionation, is first carried out, or macromolecular protein complexes are first isolated, before dye labeling, protein separation and analysis.

Target proteins include all cellular proteins and/or proteins secreted in biological fluids. Exemplary target proteins include pumps, regulatory proteins such as receptors and transcription factors, as well as structural proteins and enzymes. The proteins may be from any organisms, including prokaryotes and eukaryotes, including, for example, enzymes from bacteria, fungi, extremeophiles, viruses, animals (particularly mammals and particularly human) and birds. Suitable classes of enzymes include, but are not limited to, hydrolases such as proteases, carbohydrases or lipases, isomerases such as racemases, epimerases, tautomerases or mutases, transferases, kinases and phophatases. Other exemplary enzymes include those that carry out group transfers, such as acyl group transfers, including endo and exopeptidases (serine, cysteine, metallo and acid proteases); amino group and glutamyl transfers, including glutaminases, γ glutamyl transpeptidases, amidotransferases, etc.; phosphoryl group transfers, including phosphatases, phosphodiesterases, kinases and phosphorylases; nucleotidyl and pyrophosphotyl transfers, including carboxylate, pyrophosphoryl transfers, etc.; glycosyl group transfers; oxidative and reductive enzymes such as dehydrogenases, monooxygenases, oxidases, hydroxylases, reductases, etc.; enzymes that catalyze eliminations, isomerizations and rearrangements, such as aconitase, fumarase, enolase, crotonase, carbon-nitrogen lyases, etc.; and enzymes that make or break carbon-carbon bonds. Suitable enzymes may be listed in the Swiss-Prot enzyme database.

Viruses which may be labeled with the optical labeling molecules described herein include, but are not limited to, orthomyxoviruses, (e.g., influenza virus), paramyxoviruses (e.g., respiratory syncytial virus, mumps virus, measles virus), adenoviruses, rhinoviruses, coronaviruses, reoviruses, togaviruses (e.g., rubella virus), parvoviruses, poxviruses (e.g., variola virus, vaccinia virus), enteroviruses (e.g., poliovirus, coxsackievirus), hepatitis viruses (including A, B and C), herpes viruses (e.g., Herpes simplex virus, varicella-zoster virus, cytomegalovirus, Epstein-Barr virus), rotaviruses, Norwalk viruses, hantavirus, arenavirus, rhabdovirus (e.g., rabies virus), retroviruses (including HIV, HTLV-I and -II), papovaviruses (e.g., papillomavirus), polyomaviruses and picornaviruses and the like).

Bacteria which may be labeled with the optical labeling molecules described herein include, but are not limited to, Bacillus; Vibrio, e.g., V. cholerae; Escherichia, e.g., Enterotoxigenic E. coli, Shigella, e.g., S. dysenteriae; Salmonella, e.g., S. typhi; Mycobacterium e.g., M. tuberculosis, M. leprae; Clostridium, e.g., C. botulinum, C. tetani, C. difficile, C. perfringens; Cornyebacterium, e.g., C. diphtheriae; Streptococcus, S. pyogenes, S. pneumoniae; Staphylococcus, e.g., S. aureus; Haemophilus, e.g., H. influenzae; Neisseria, e.g., N. meningitidis, N. gonorrhoeae; Yersinia, e.g., G. lamblia Y. pestis, Pseudomonas, e.g., P. aeruginosa, P. putida; Chlamydia, e.g., C. trachomatis; Bordetella, e.g., B. pertussis; Treponema, e.g., T. palladium; and the like.

Cell types or cell lines which may be labeled with the optical labeling molecules described herein include, but are not limited to, disease state cell types, (e.g., tumor cells of all types (particularly, melanoma, myeloid leukemia, carcinomas of the lung, breast, ovaries, colon, kidney, prostate, pancreas and testes)), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Other exemplary cells also include known research cell lines, such as, Jurkat T cells, NIH3T3 cells, CHO, Cos, etc. which may be found in the ATCC cell line catalog. In some embodiments, the cells may be genetically engineered, that is, contain exogeneous nucleic acid, for example, when the effect of additional genes or regulatory sequences on expressed proteins is to be evaluated.

The optical labeling molecules described herein may be used to label other cellular components, such as carbohydrates, lipids, and nucleic acids, including DNA and RNA.

In some embodiments, the activator forms a covalent bond with an amine group of a target protein. Examples of activators that form covalent bonds with amine groups are imidoesters, N-hydroxysuccinimidyl esters, sulfosuccinimidyl esters, isothiocyanates, aldehydes, sulfonylchlorides, or arylating agents. Amine groups are present in several amino acids, including lysine. Lysine E-amino groups are common in proteins (typically 6-7/100 of the residues) and typically many lysine residues are located on protein surfaces and thus are accessible to optical labeling molecules. In some embodiments, the N-terminal amino groups of proteins may be pre-labeled near neutral pH with a different amine-reactive group, such as a small acid anhydride with or without an isotopic label to minimize dye-induced shifts in isoelectric focusing after lysine labeling.

In other embodiments, thiol groups of the target protein are used as the activator attachment site. The thiol groups can either be present in proteins or be produced (after thiol protection) by chemical treatment of —SNO groups or sulfenic acid groups. Examples of activators that form covalent bonds with thiol groups and thiol post-translational modifications are sulfhydryl-reactive maleimides, iodoacetamides, alkyl bromides, vinyl pyridines, pyridine disulfides, methyl methanethiosulphonate, cyclohexanedione, benzoxidiazoles, and

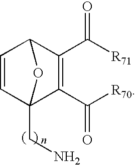

In some embodiments, post-translational modifications on the target protein are used as the activator site. For example, the activator may be a boronic acid moiety for coordination to a carbohydrate modification and a benzophenone moiety for photochemically induced covalent attachment.

In other embodiments, other posttranslationally modified chemical groups on the target proteins are used as the activator attachment site. Examples of reactive groups are those produced by beta elimination of phosphates or O-linked carbohydrate groups that can then be reacted with thiol groups on the fluorescent dye compounds or to linkers that react with the beta elimination site and then to an activator on reactive fluorescent dye compounds.

In some embodiments, the active site of an enzyme may the target for the activator. In these cases, inhibitors selective for the active site of enzymes are employed to covalently bind the active site or coordinate the active site with subsequent photochemical binding to benzophenone. Such binding allows for the detection of enzyme activity levels.

In some embodiments, in addition to a zwitterionic dye moiety, an optional titratable group and an activator, the optical labeling molecule also includes a cleavable moiety. By “cleavable moiety” is meant a group that can be chemically, photochemically, or enzymatically cleaved. In some embodiments, the cleavable moiety forms a stable bond but can be efficiently cleaved under mild, physiological, conditions. In other embodiments, the cleavable moiety is a photocleavable moiety. In still other embodiments, the photocleavable moiety is an O-nitrobenzylic compound, which can be synthetically incorporated into the zwitterionic labeling dye via an ether, thioether, ester (including phosphate esters), amine or similar linkage to a heteroatom (particularly oxygen, nitrogen or sulfur). Also useful are benzoin-based photocleavable moieties and nitrophenylcarbamate esters. A wide variety of suitable photocleavable moieties may be found in the Molecular Probes Catalog, supra.

The cleavable moiety increases the maximum detection sensitivity of the optical labeling molecule by allowing a high multiplicity of dye labeling which is then followed by removal of the optical labeling molecule prior to further analysis. For example, the optical labeling molecule can be removed after protein separation via removal of the cleavable moiety prior to mass spectroscopy (MS) analysis.

Identification of interesting protein spots on 2D gels for further study is typically accomplished by fluorescent scanning during gel analysis, but protein identification is generally accomplished by mass spectrometry. The most generally effective method of identifying proteins and posttranslational modifications thereof involves digesting proteins with trypsin or lysine-specific enzymes, before analysis by mass spectrometry. As is well known in the art, trypsin is an enzyme that specifically cleaves at the basic amino acid groups, arginine and lysine. High multiplicity attachment of optical labeling molecules will label most of the accessible lysine amino groups and will thus prevent trypsin digestion at these sites. In some embodiments the thiol groups on the proteins are saturated with optical labeling molecules to increase the detection sensitivity. In some embodiments, the optical labeling molecule is removed from the protein after separation by chemical, photochemical or enzymatic methods.

In some embodiments, the optical labeling molecule includes a second label in addition to the zwitterionic dye. The second label can be, for example, a stable isotope label, an affinity tag, an enzymatic label, a magnetic label or a second fluorophore. In some embodiments, the optical labeling moiety is a zwitterionic dye moiety, a linker, an optional titratable group, a cleavable moiety, a stable isotope moiety and an activator. In other embodiments, the stable isotope moiety is a light isotope. In still other embodiments, embodiments, the stable isotope moiety is one or more combinations of heavy isotopes. In still other embodiments, the stable isotope moiety is located between the cleavable moiety and the activator.

In some embodiments, the optical labeling molecule has a zwitterionic dye moiety, a linker, an optional titratable group, a cleavable moiety, a stable isotope moiety and an activator. In this embodiment, cleavage of the cleavable moiety results in labeling the protein with the stable isotope moiety. Accordingly, the relative or absolute amount of the protein expressed by the biological system under different stimulus conditions can be quantitated, using isotope ratios in a mass spectrometer as is well known to those of skill in the art.

In some embodiments, an optical labeling molecule of structural Formula (I), or a salt or solvate thereof, is provided:

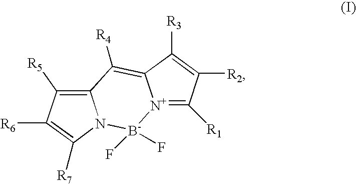

wherein the optical labeling molecule comprises a fluorophore with a derivative tail, and the derivative tail comprises at least two amide bonds; wherein:
R₁-R₇ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —(CH₂)ₙN⁺(CH₃)₃, —SO₃H, —SO₃H, —(CH₂)ₙS(O)ₙOH, —(CH₂)ₙS(O)₂O⁻, —OP(O)(O⁻)₂, —(CH₂)ₙOP(O)(O⁻)₂;

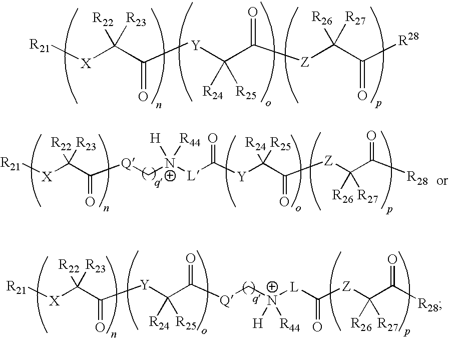

provided that at least one and only one of R₁-R₇ is

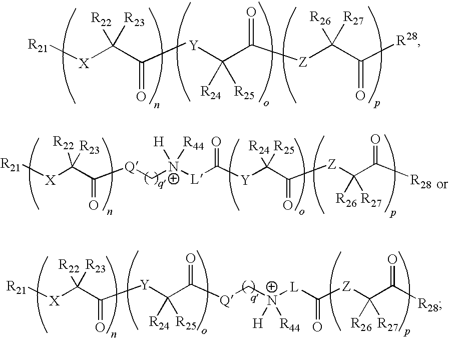

R₂₂, R₂₃, R₂₄, R₂₅, R₂₆ and R₂₇ are independently, hydrogen, alkyl, substituted alkyl, amino, aryl, substituted aryl, arylalkyl, substituted arylalkyl, heteroaryl, substituted heteroaryl, heteroarylalkyl, heteroalkyl or substituted heteroalkyl; Halo; nitro; —(CH₂)ₙN⁺(CH₃)₃; —S(O)ₙR₂₀; —SO₃H; —(CH₂)ₙS(O)OH; —(CH₂)ₙS(O)₂O⁻; OP(O)(O⁻)₂, —CH₂OP(O)(O⁻)₂;
R₂₁ is —(CH₂)ₘ—C(O)—, —(CH₂)ₘ—C(O)-Q′(CH₂)_{q}—N⁺H(R₄₆)-L′-C(O)—

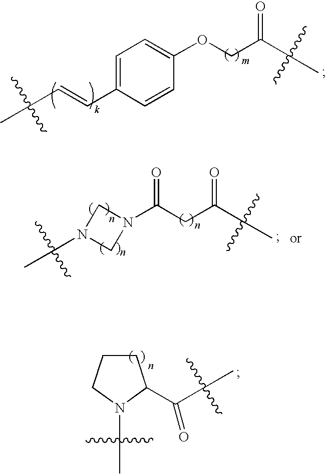

R₂₈ is -Q-L-C(O)-A; -Q(CH₂)_{q}—N⁺H(R₄₅)-L-C(O)-A, -Q-L-D-C(O)—(B′), A or -Q(CH₂)_{q}—N⁺H(R₄₅)-L-D-C(O)—(B′)ᵣ-A;
R₂₀ and R₄₃ are independently alkyl, substituted alkyl, aryl, substituted aryl, arylalkyl, substituted arylalkyl, heteroaryl, substituted heteroaryl, heteroarylalkyl, heteroalkyl or substituted heteroalkyl;
R₂₉, R₃₄ and R₄₄-R₄₇ are independently hydrogen, alkyl or substituted alkyl;
k and m are independently 1, 2, 3, 4 or 5;
n, o and p are independently 0, 1, 2, 3, 4 or 5;
q and q′ are independently 2, 3, 4 or 5;
e and t are independently 0, 1 or 2;
Q is —NR₂₉;
X is —NR₃₀ or —O—;
Y is —NR₃, or —O—;
Z is —NR₃₂ or —O—;
Q′ is —NR₃₃;
B′ is —NH—C(R₃₄)—C(O)— wherein R₃₄ is hydrogen, alkyl, substituted alkyl, aryl, substituted aryl, arylalkyl, substituted arylalkyl, heteroaryl, substituted heteroaryl, heteroarylalkyl, heteroalkyl or substituted heteroalkyl;
r is 0 or 1;
L and L′ are alkyl, substituted alkyl, heteroalkyl or substituted alkyl, aryl or substituted aryl;
A is OH, —NHCH₂CH₂SH,

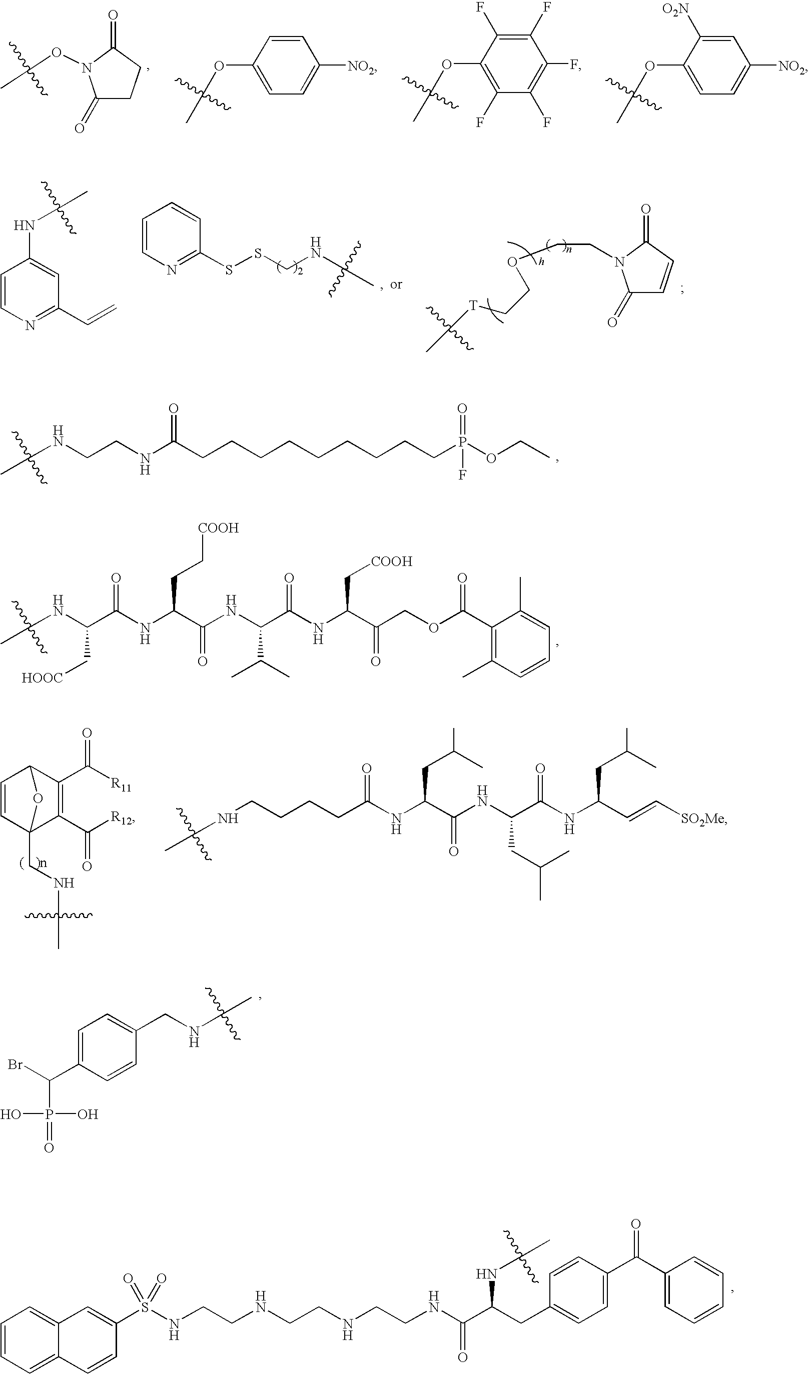

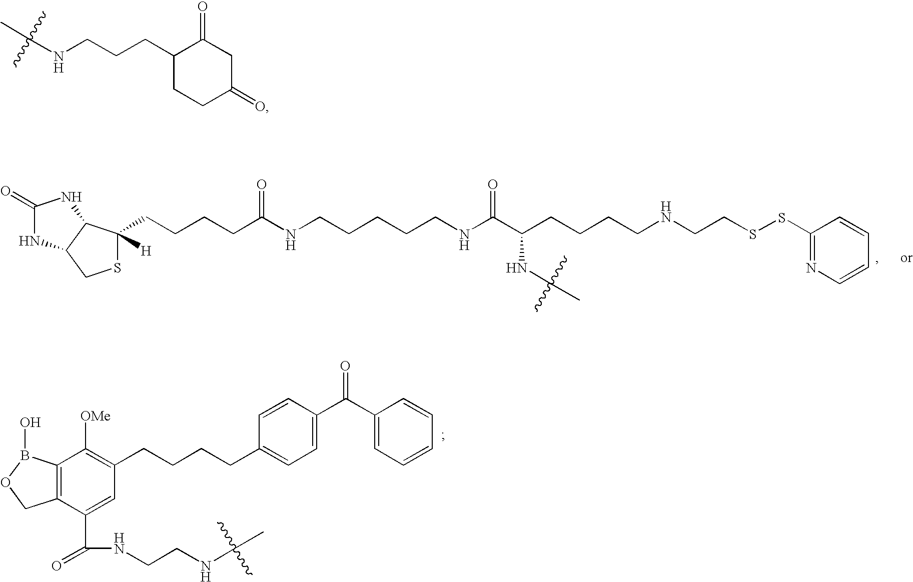

R₁₁ and R₁₂ are independently alkyl, substituted alkyl, acyl, substituted acyl, alkoxy, substituted alkoxy, aryl, substituted aryl, azido alkyl, alkynyl, substituted alkynyl, amino, or substituted amino.
T is —NR₃₄;
D is

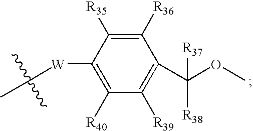

R₃₇ and R₃₈ are independently hydrogen, alkyl or substituted alkyl;
R₃₅, R₃₆, R₃₉ and R₄₀ are independently hydrogen, nitro, alkyl, substituted alkyl, —NR₄₁R₄₂, —S(O)ₜR₄₃, aryloxy, substituted aryloxy, alkoxy or substituted alkoxy provided that at least one of R₃₅, R₃₆, R₃₇ and R₃₈ is nitro, aryloxy, substituted aryloxy, alkoxy or substituted alkoxy; and
W is —O—, —S— or —NR₄₇;
provided that R₁-R₇ includes at least one zwitterionic pair.

In other embodiments, R₁-R₇ are independently hydrogen, alkyl, substituted alkyl, aryl, substituted aryl, heteroaryl, substituted heteroaryl or

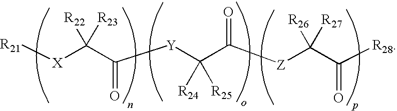

In still other embodiments, R₁ is

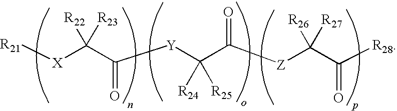

In still other embodiments, R₂, R₃, R₄, R₅, R₆ and R₇ are independently hydrogen, alkyl, substituted alkyl, aryl, substituted aryl, heteroaryl or substituted heteroaryl and R₁ is

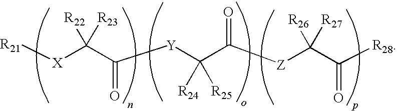

In still other embodiments, R₃ is alkyl or substituted alkyl, R₅ is alkyl or substituted alkyl, R₇ is aryl or substituted aryl or heteroaryl or substituted heteroaryl and R₂, R₄ and R₆ are hydrogen and R₁ is

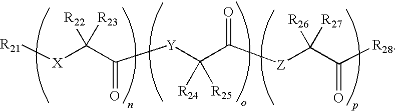

In still other embodiments, R₃, R₅, and R₇ are independently the same or different alkyl or substituted alkyl, R₂, R₄, and R₆ are hydrogen and R₁ is

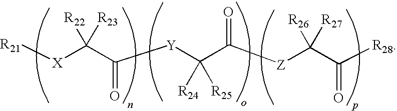

In still other embodiments, R₃, R₅, and R₇ are independently the same or different alkyl or substituted alkyl, R₄ is aryl, substituted aryl, heteroaryl, or substituted heteroaryl, R₂ and R₆ are hydrogen and R₁ is

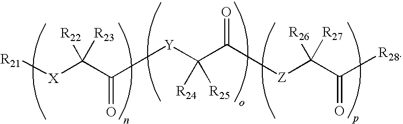

In still other embodiments, R₁, R₅, and R₇ are independently the same or different alkyl or substituted alkyl, R₂, R₄, and R₆ are hydrogen and R₃ is

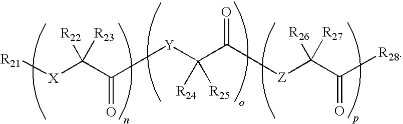

In still other embodiments, R₁ and R₅ are independently the same or different alkyl or substituted alkyl, R₇ is aryl or substituted aryl, heteroaryl or substituted heteroaryl, R₂, R₄, and R₆ are hydrogen, and R₃ is

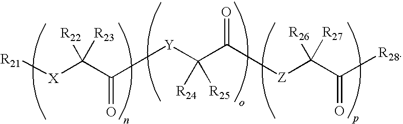

In still other embodiments, R₁, R₄, and R₇ are independently the same or different alkyl or substituted alkyl, aryl or substituted aryl, heteroaryl or substituted heteroaryl, R₅ is alkyl or substituted alkyl, R₂ and R₆ are hydrogen and R₃ is

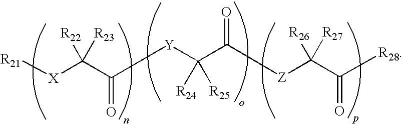

In some embodiments, R₃ is methyl, propyl or —(CH₂)₄N⁺(CH₃)₃. In other embodiments, R₅ is methyl, propyl or —(CH₂)₄N⁺(CH₃)₃. In still other embodiments, R₇ is phenyl, p-methoxyphenyl, thiophenyl, methyl, propyl, butyl, heptyl, or —(CH₂)₄N⁺(CH₃)₃

In some embodiments, R₃ is methyl, propyl or —(CH₂)₄N⁺(CH₃)₃, R₅ is methyl, propyl, or —(CH₂)₄N⁺(CH₃)₃, R₇ is phenyl, p-methoxyphenyl, thiophenyl, methyl, propyl, butyl, heptyl or —(CH₂)₄N⁺(CH₃)₃, R₂, R₄ and R₆ are hydrogen and R₁ is

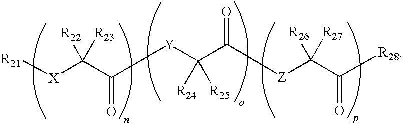

In other embodiments, R₃ is methyl, R₅ is —(CH₂)₄N⁺(CH₃)₃, R₇ is phenyl, p-methoxyphenyl, thiophenyl, methyl, propyl, butyl, heptyl, or —(CH₂)₄N⁺(CH₃)₃, R₂, R₄ and R₆ are hydrogen and R₁ is

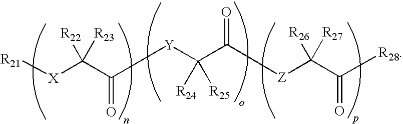

In still other embodiments, R₃ is —(CH₂)₄N⁺(CH₃)₃, R₅ is —(CH₂)₄N⁺(CH₃)₃, R₇ is phenyl, p-methoxyphenyl, methyl, propyl, butyl, heptyl, or —(CH₂)₄N⁺(CH₃)₃, R₂, R₄ and R₆ are hydrogen and R₁ is

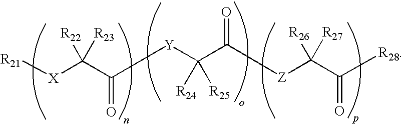

In still other embodiments, R₃ propyl, R₅ is propyl, R₇ is —(CH₂)₄N⁺(CH₃)₃, R₂, R₄ and R₆ are hydrogen and R₁ is

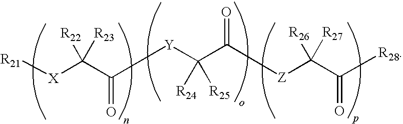

In some of the above embodiments, R₂₁ is —(CH₂)ₘ—C(O)—, n is 1, X is —NH—, o is 0, p is 1, Z is —NH— and R₂₈ is -Q-L-C(O)-A. In some of the above embodiments, R₂₁ is —(CH₂)₂—C(O)—, n is 1, X is —NH—, R₂₂ is hydrogen, R₂₃ is —CH₂SO₃— or —(CH₂)₄N⁺(CH₃)₃, o is 0, p is 1, Z is —NH—, R₂₆ and R₂₇ are hydrogen, R₂₈ is -Q-L-C(O)-A and Q is —NH—, L is —(CH₂)₄—. In some of the above embodiments, R₂₁ is —(CH₂)ₙ—C(O)—, n is 1, X is —NH—, o is 0, p is 0 and R₂₈ is -Q(CH₂)_{q}—N⁺H(R₂₁)-L-D-C(O)—(B)ᵣ-A. In some of the above embodiments, R₂₁ is —(CH₂)₂—C(O)—, n is 1, X is —NH—, R₂₂ is hydrogen, R₂₃ is —CH₂SO₃— or —(CH₂)₄N⁺(CH₃)₃, o is 0, p is 0, R₂₈ is -Q(CH₂)₂—N⁺H(R₂₁)-L-D-C(O)-A, Q is —NH—, L is —(CH₂)₂— and R₂₁ is methyl.

In some of the above embodiments, D is

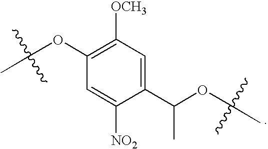

In some of the above embodiments, r is 1 and R₃₄ is hydrogen.

In some of the above embodiments, R₂₁ is n is 1, X is —NH—, o is 1, Y is —NH— and R₂₈ is -Q-L-C(O)-A. In some of the above embodiments, R₂₁ is —(CH₂)₂—C(O)—, R₂₂ is hydrogen, R₂₃ is —CH₂SO₃— or —(CH₂)₄N⁺(CH₃)₃, R₂₄ is hydrogen, R₂₅ is —CH₂SO₃— or —(CH₂)₄N⁺(CH₃)₃, n is 1, X is —NH—, o is 1, Y is —NH—, R₂₈ is -Q-L-C(O)-A, Q is —NH—, L is —(CH₂)₄— and R₂₁ is methyl. In some of the above embodiments, R₂₁ is —(CH₂)ₘ—C(O)—, n is 1, X is —NH—, o is 1, Y is —NH—, p is 0 and R₂₈ is -Q(CH₂)_{q}—N⁺H(R₄₅)-L-D-C(O)—(B)ᵣ-A. In some of the above embodiments, R₂₁ is —(CH₂)₂—C(O)—, n is 1, X is —NH—, o is 1, Y is —NH—, R₂₂ is hydrogen, R₂₃ is —CH₂SO₃— or —(CH₂)₄N⁺(CH₃)₃, R₂₄ is hydrogen, R₂₅ is —CH₂SO₃— or —(CH₂)₄N⁺(CH₃)₃, p is 0, R₂₈ is -Q(CH₂)₂—N⁺H(R₄₅)-L-D-C(O)-A, Q is —NH—, L is —(CH₂)₂— and R₄₅ is methyl.

In some of the above embodiments, D is

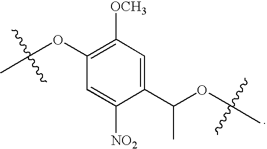

In some of the above embodiments, r is 1 and R₃₄ is hydrogen.

In some of the above embodiments, R₂₁ is

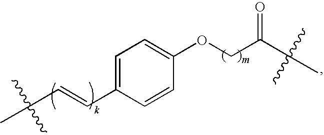

n is 1, X is —NH—, o is 0, p is 1, Z is —NH— and R₂₈ is -Q-L-C(O)-A. In some of the above embodiments, R₂₁ is

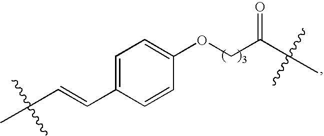

n is 1, X is —NH—, R₂₂ is hydrogen, R₂₃ is —CH₂SO₃— or —(CH₂)₄N⁺(CH₃)₃, o is 0, p is 1, Z is —NH—, R₂₆ and R₂₇ are hydrogen, R₂₈ is -Q-L-C(O)-A, Q is —NH— and L is —(CH₂)₄—. In some of the above embodiments, R₂₁ is

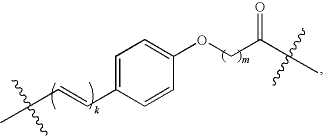

n is 1, X is —NH—, o is 0, p is 0 and R₂₈ is -Q(CH₂)_{q}—N⁺H(R₄₅)-L-D-C(O)—(B)ᵣ-A. In some of the above embodiments, R₂₁ is

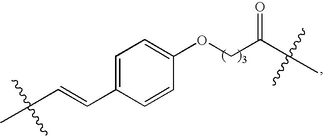

n is 1, X is —NH—, R₂₂ is hydrogen, R₂₃ is —CH₂SO₃— or —(CH₂)₄N⁺(CH₃)₃, o is 0, p is 0, R₂₈ is -Q(CH₂)₂—N⁺H(R₄₅)-L-D-C(O)-A, Q is —NH—, L is —(CH₂)₂— and R₄₅ is methyl. In some of the above embodiments, D is

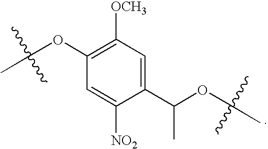

In some of the above embodiments, r is 1 and R₃₄ is hydrogen. In some of the above embodiments, R₂₁ is

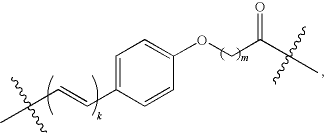

n is 1, X is —NH—, o is 1, Y is —NH— and R₂₈ is -Q-L-C(O)-A. In some of the above embodiments, R₂₁ is

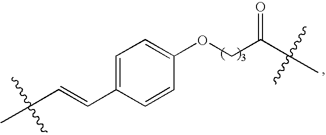

R₂₂ is hydrogen, R₂₃ is —CH₂SO₃— or —(CH₂)₄N⁺(CH₃)₃, R₂₄ is hydrogen, R₂₅ is —CH₂SO₃— or —(CH₂)₄N⁺(CH₃)₃, n is 1, X is —NH—, o is 1, Y is —NH— and R₂₈ is -Q-L-C(O)-A, Q is —NH—, L is —(CH₂)₄— and R₂₁ is methyl. In some of the above embodiments, R₂₁ is

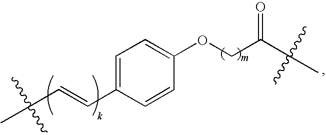

n is 1, X is —NH—, o is 1, Y is —NH—, p is 0 and R₂₈ is -Q(CH₂)_{q}—N⁺H(R₄₅)-L-D-C(O)—(B)ᵣ-A.
In some of the above embodiments, R₂₁ is

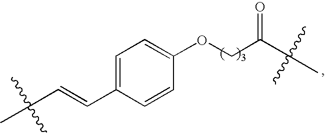

n is 1, X is —NH—, o is 1, Y is —NH—, R₂₂ is hydrogen, R₂₃ is —CH₂SO₃— or —(CH₂)₄N⁺(CH₃)₄, R₂₄ is hydrogen, R₂₅ is —CH₂SO₃— or —(CH₂)₄N⁺(CH₃)₃, p is 0, R₂₈ is -Q(CH₂)₂—N⁺H(R₄₅)-L-D-C(O)-A, Q is —NH—, L is —(CH₂)₂— and R₄₅ is methyl. In some of the above embodiments, D is

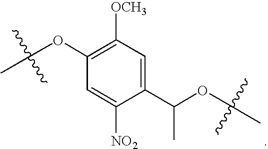

In some of the above embodiments, r is 1 and R₃₄ is hydrogen. In some embodiments, the compounds of Formula (I) include the compounds of Table 1.

TABLE 1

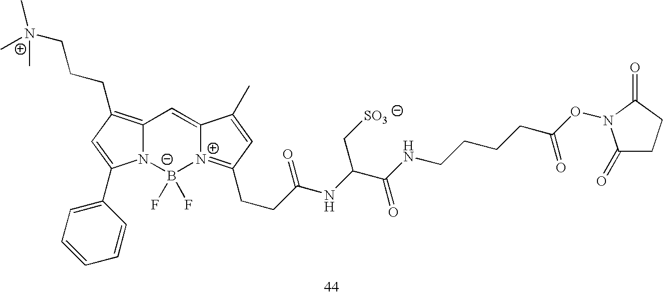

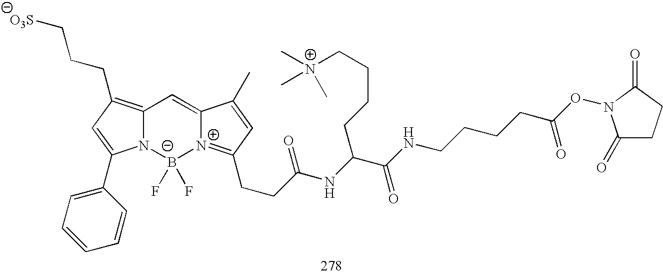

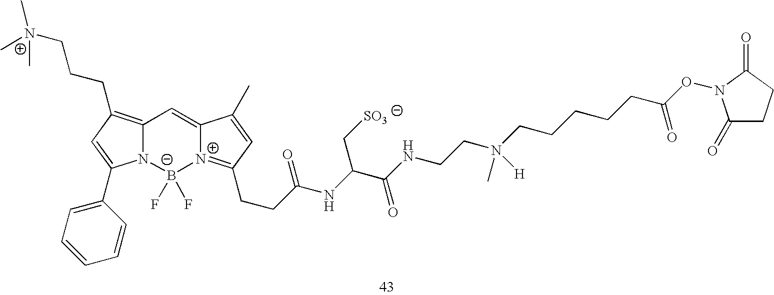

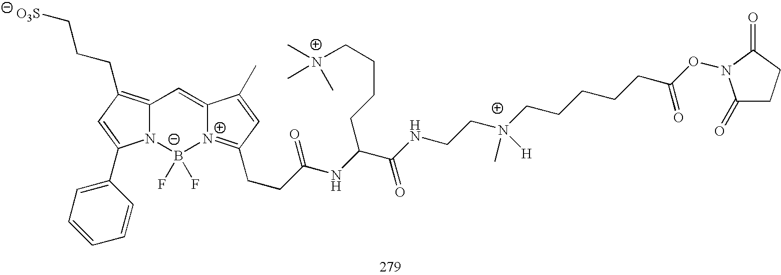

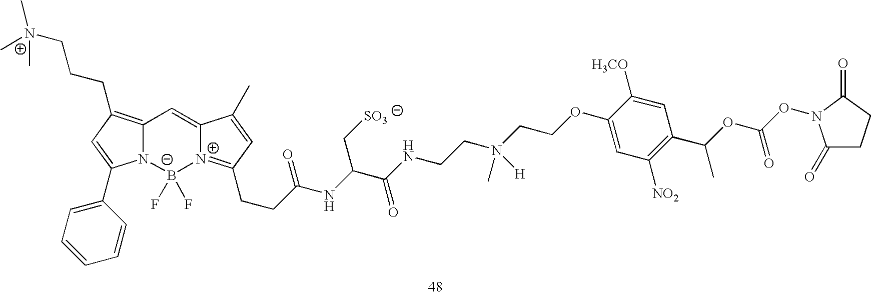

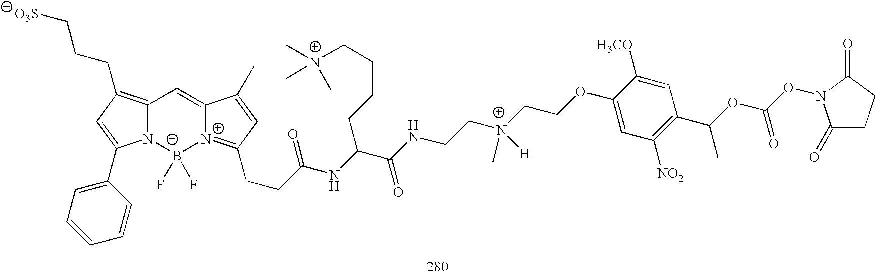

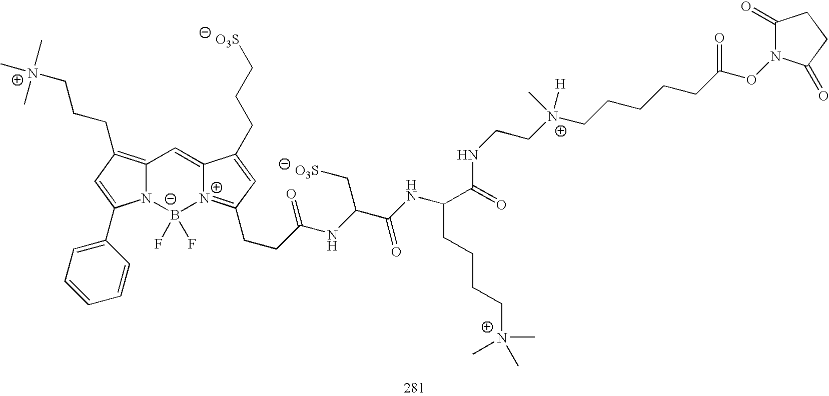

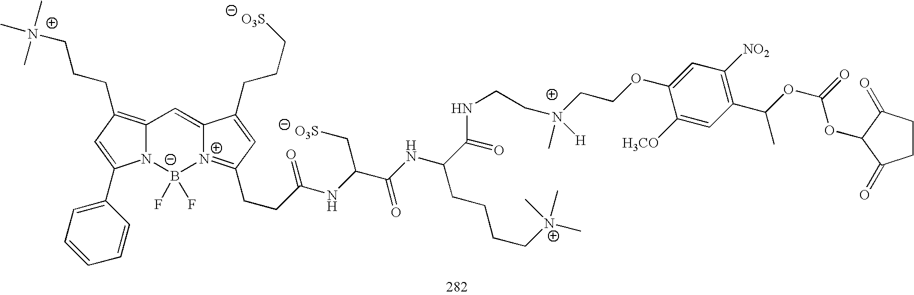

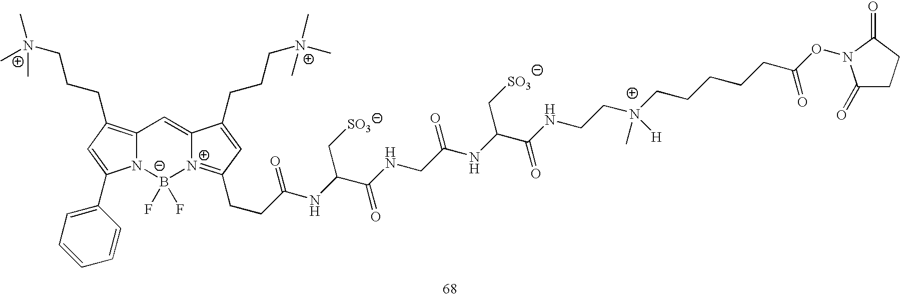

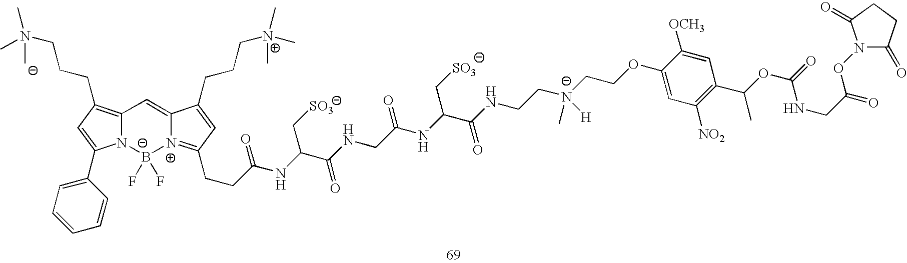

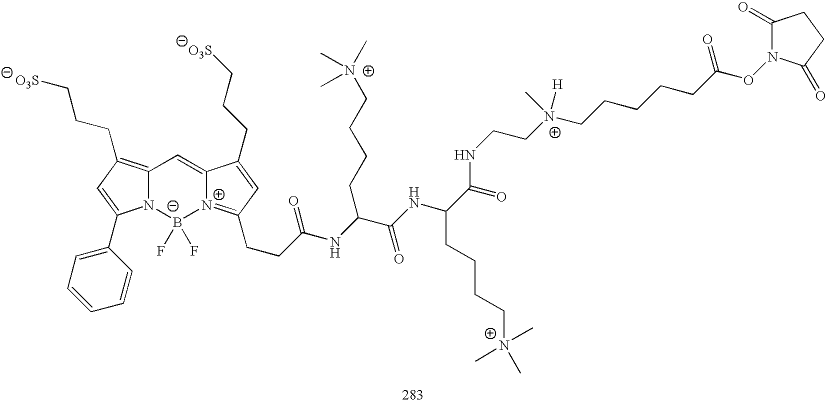

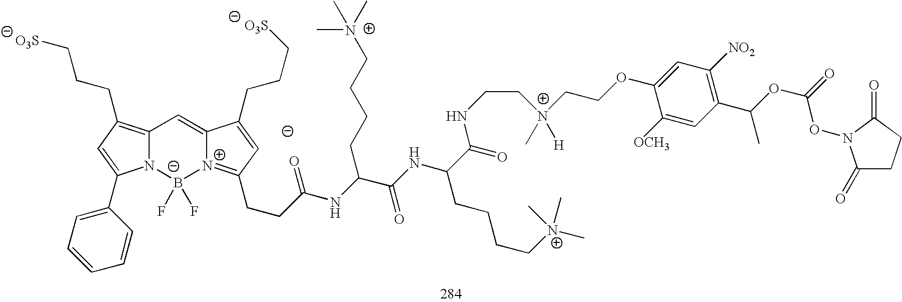

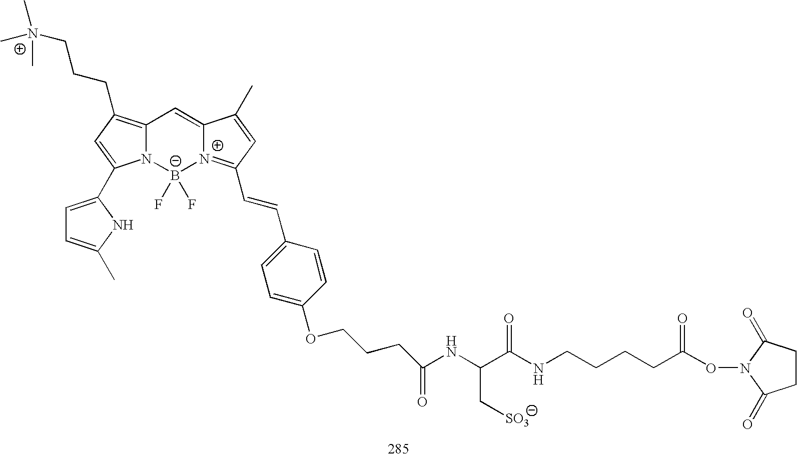

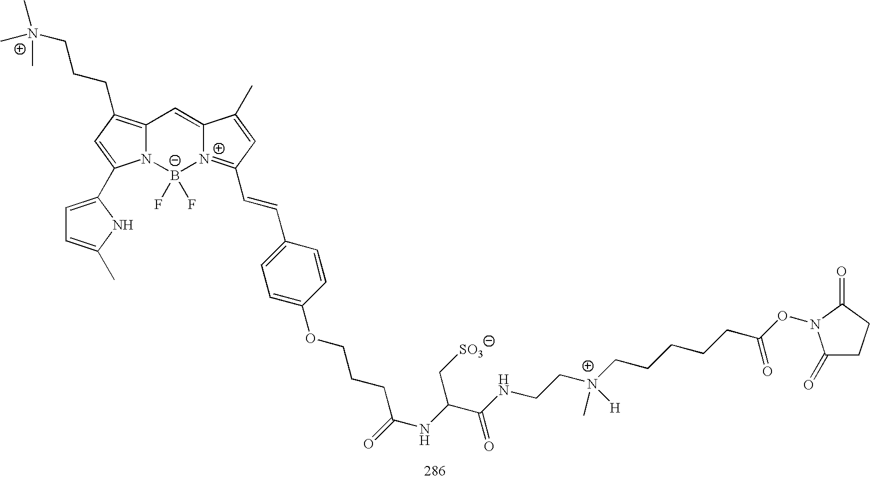

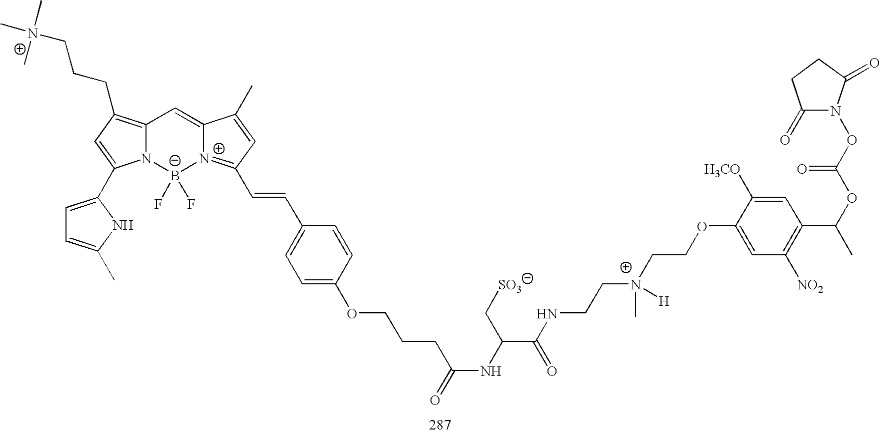

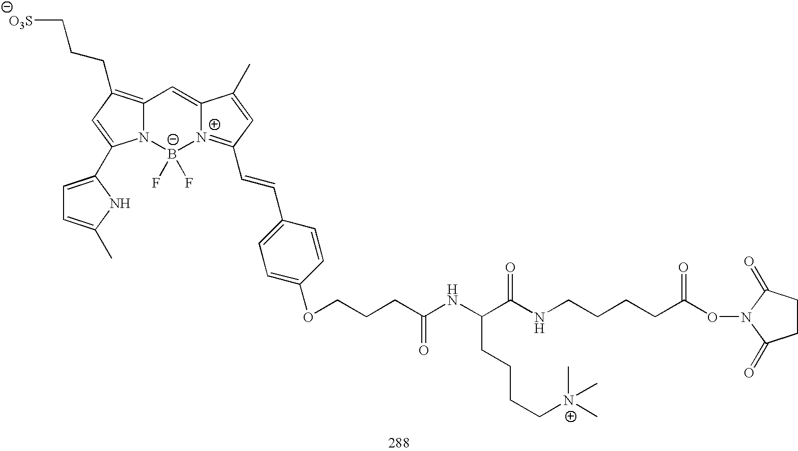

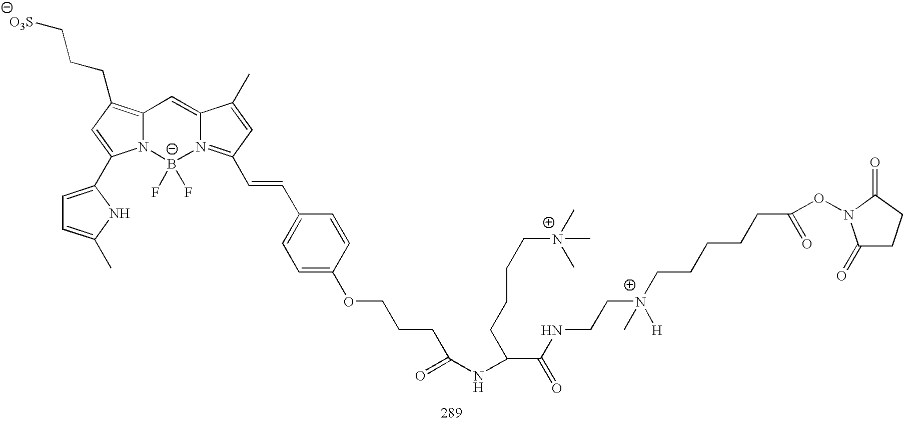

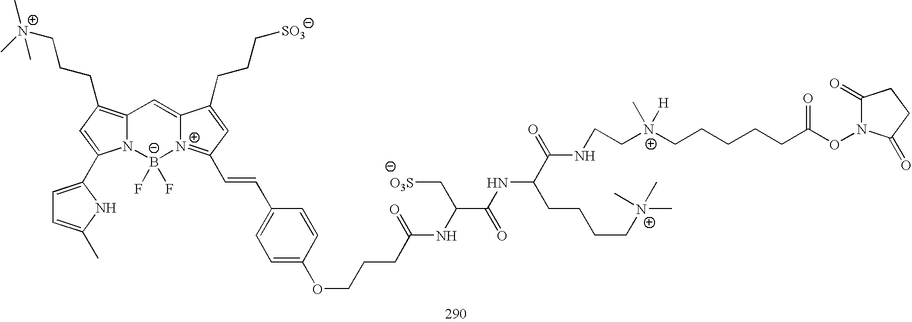

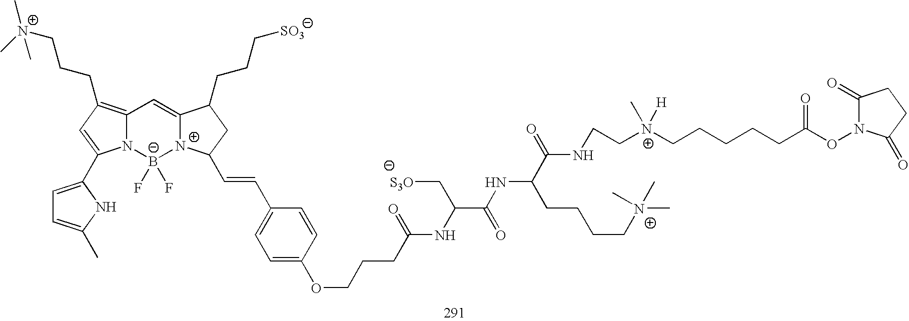

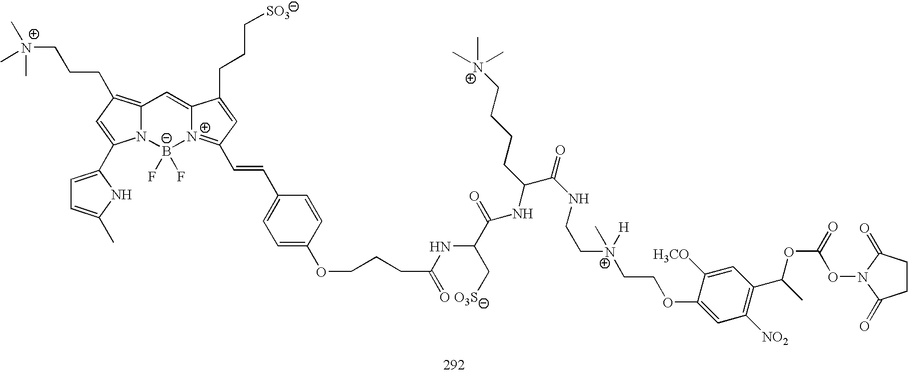

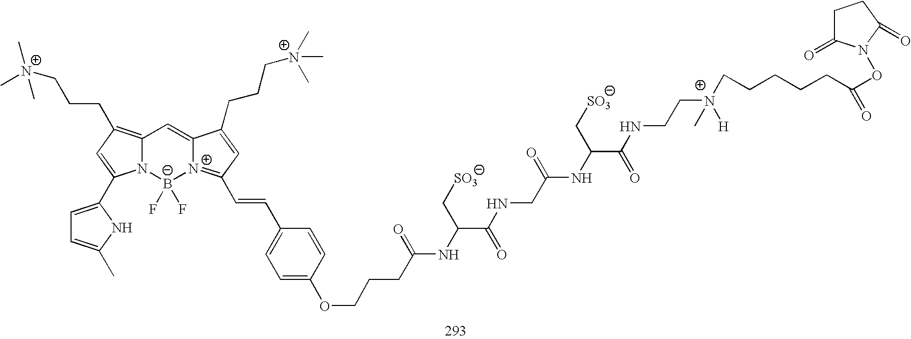

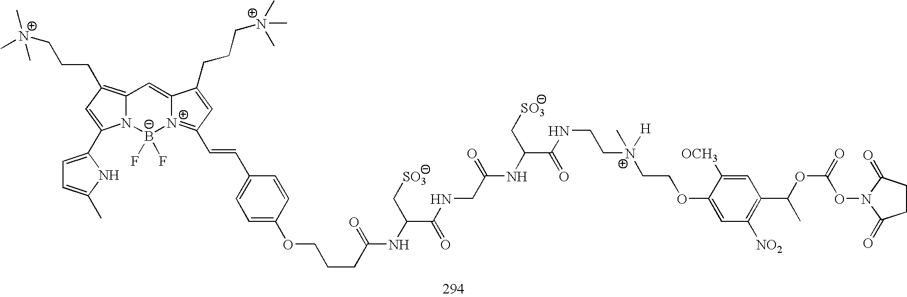

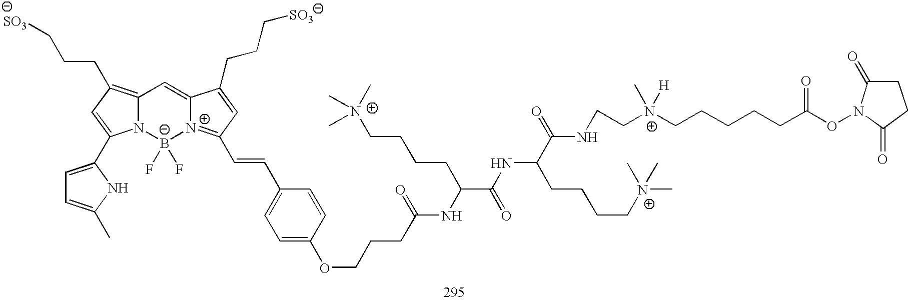

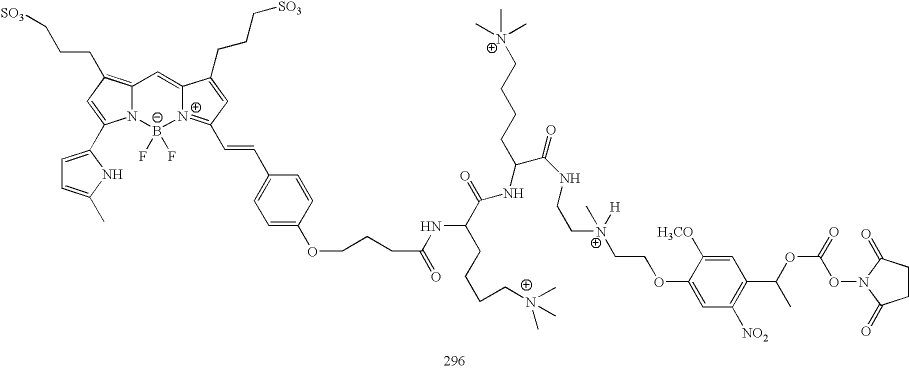

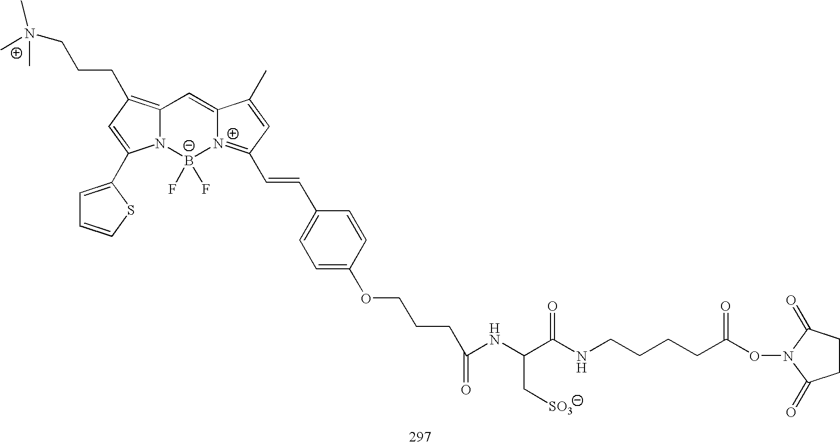

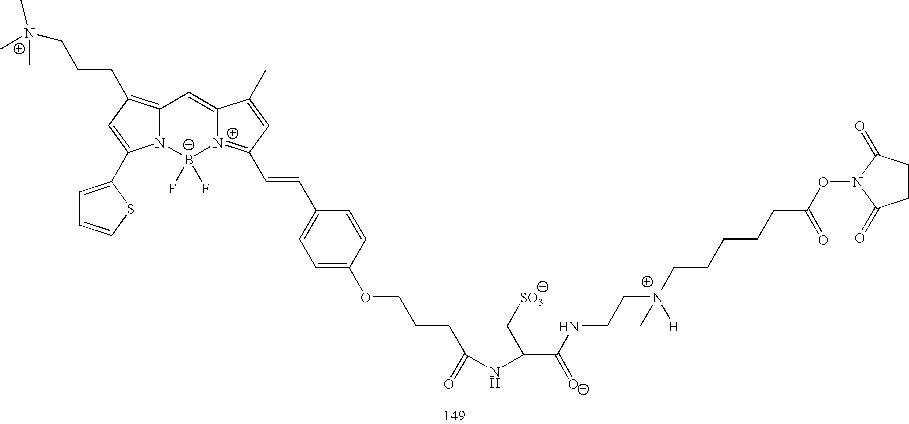

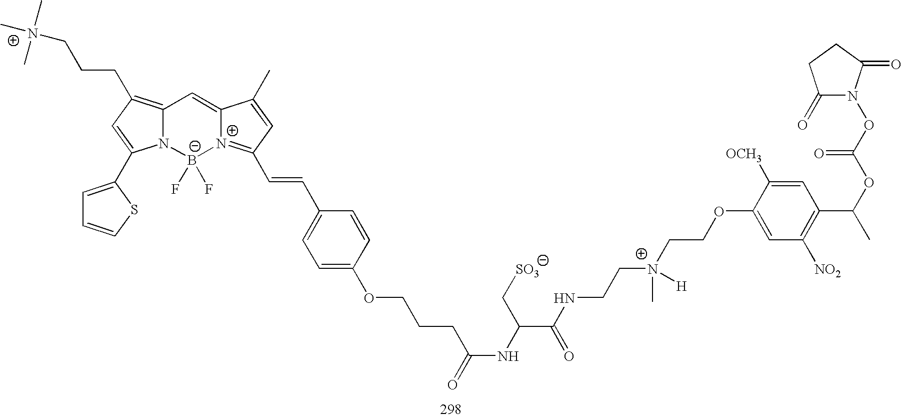

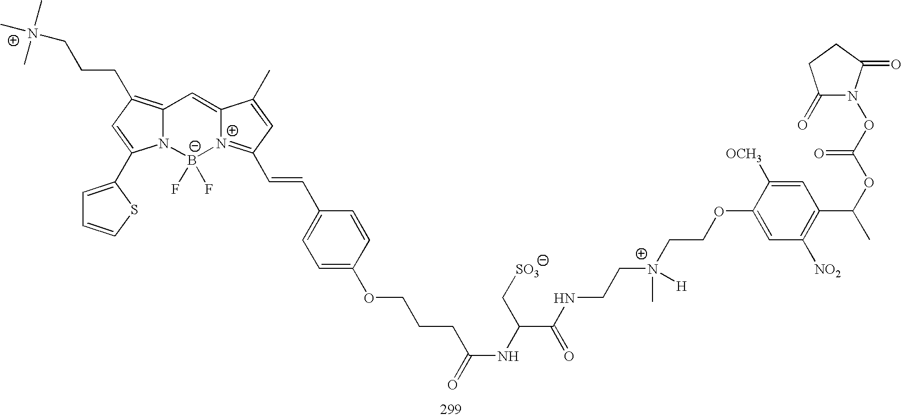

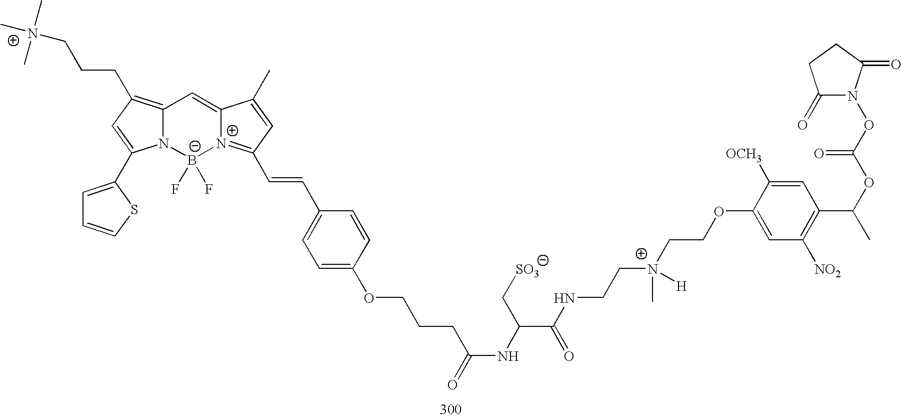

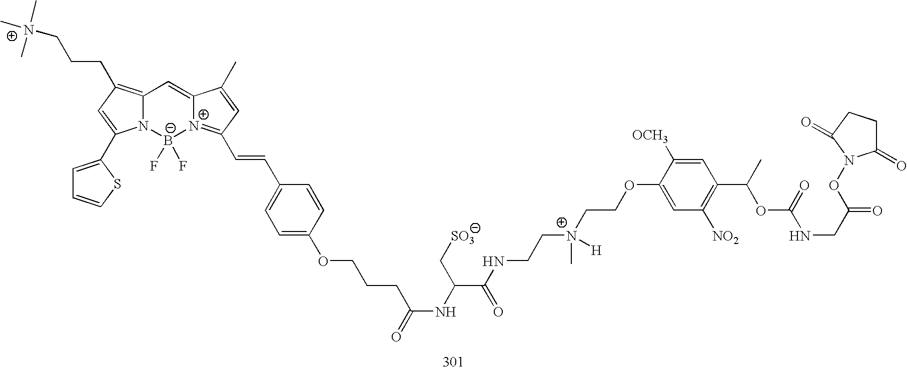

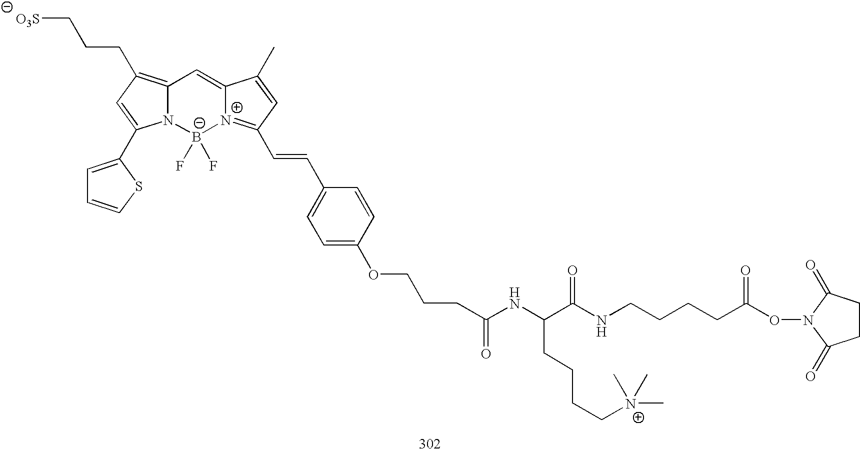

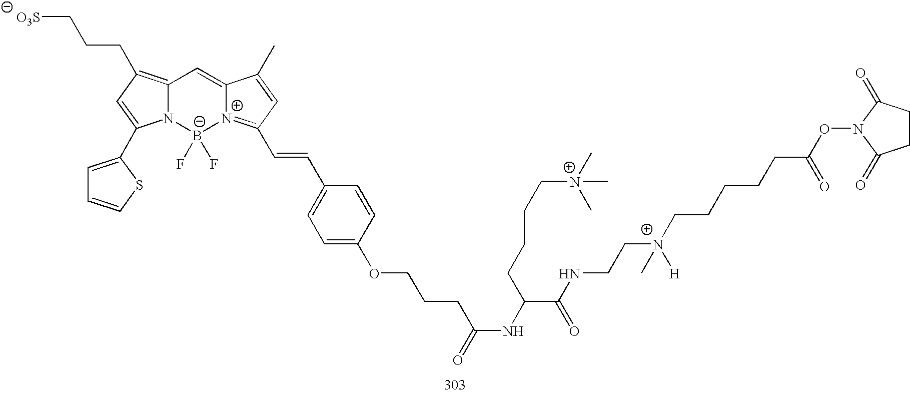

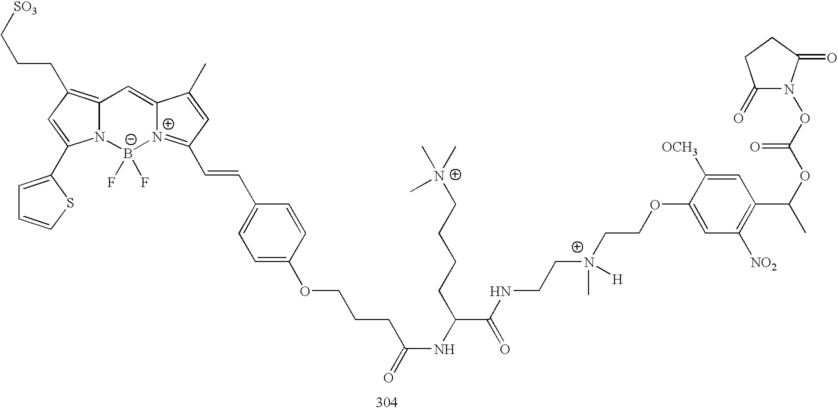

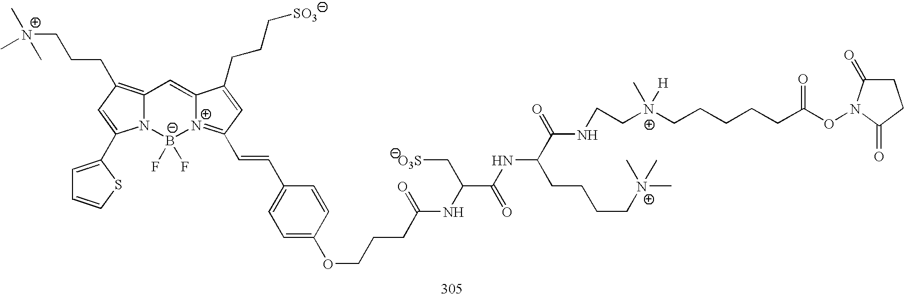

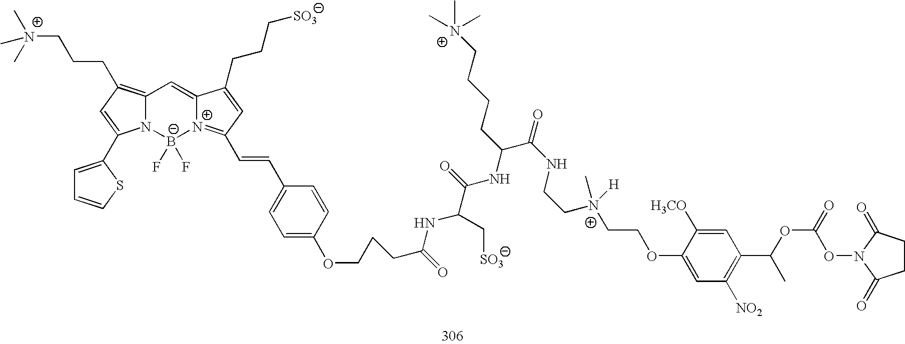

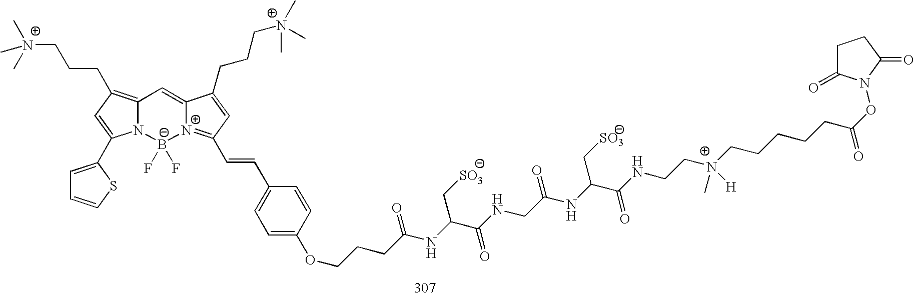

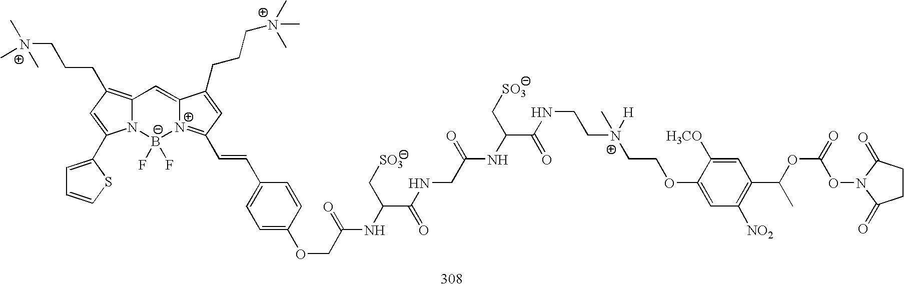

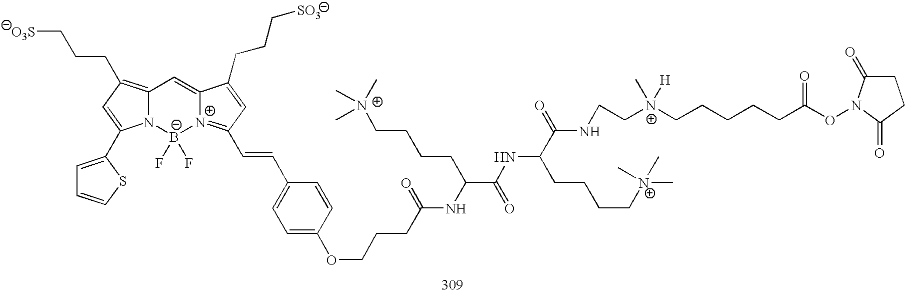

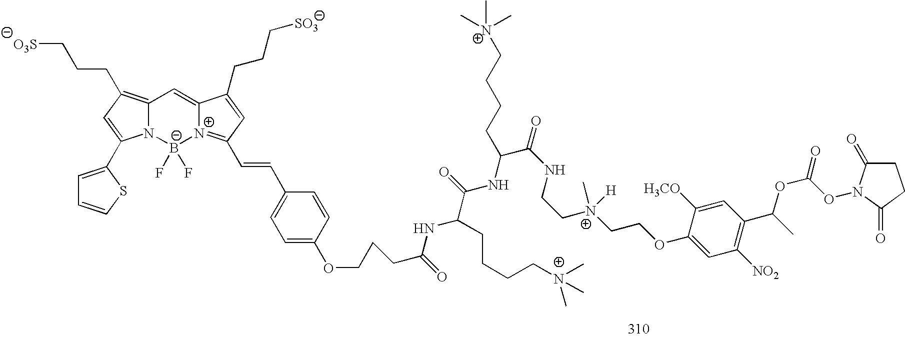

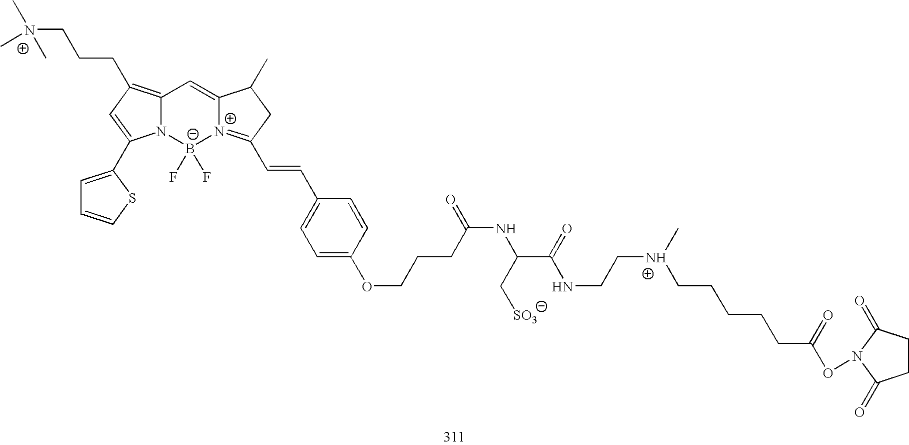

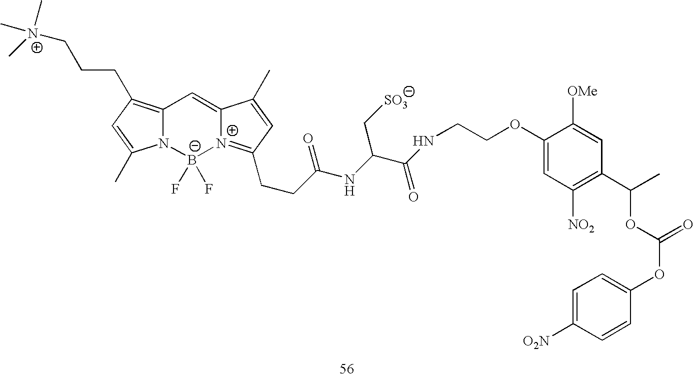

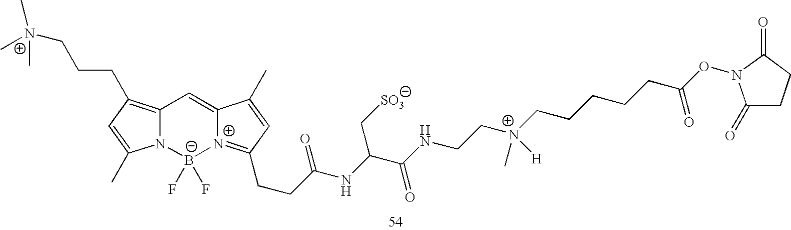

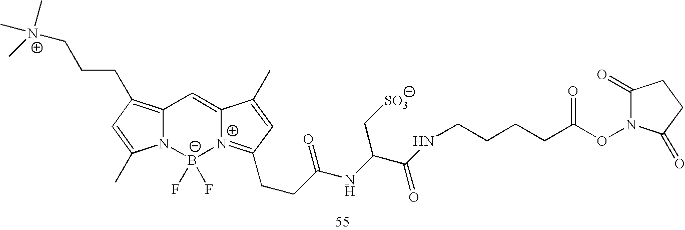

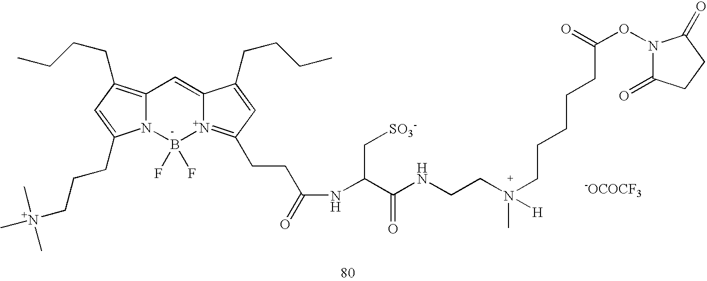

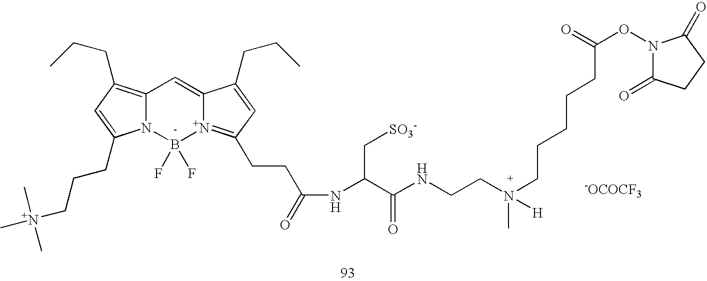

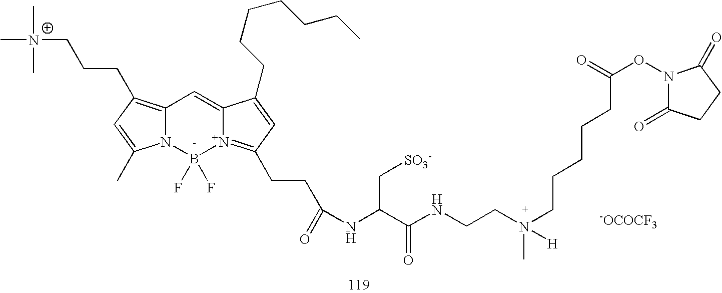

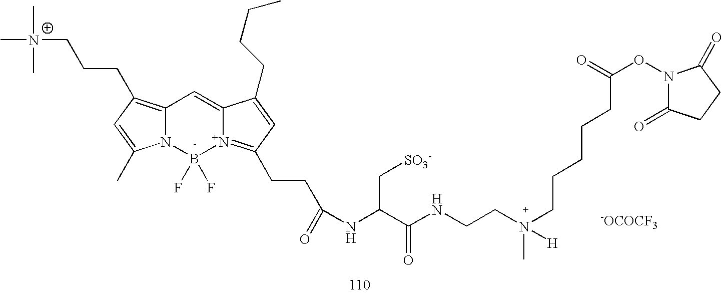

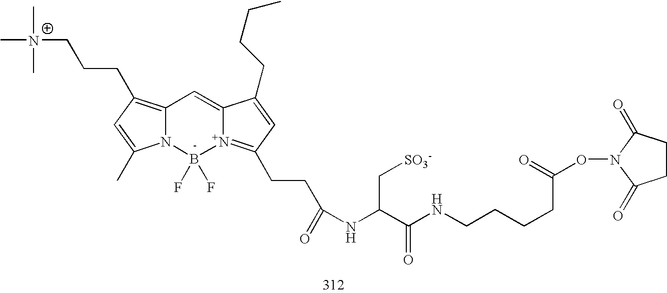

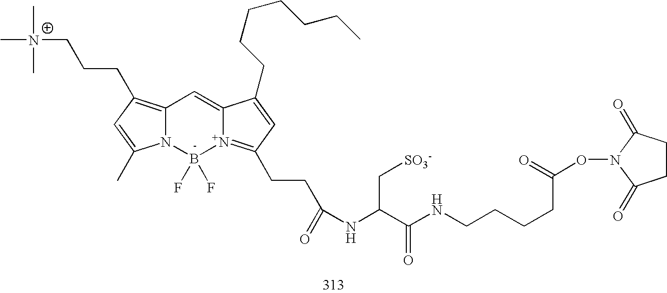

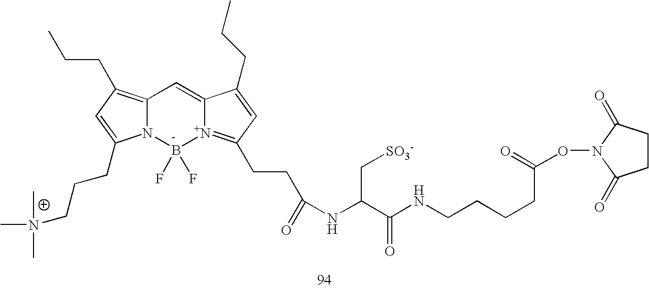

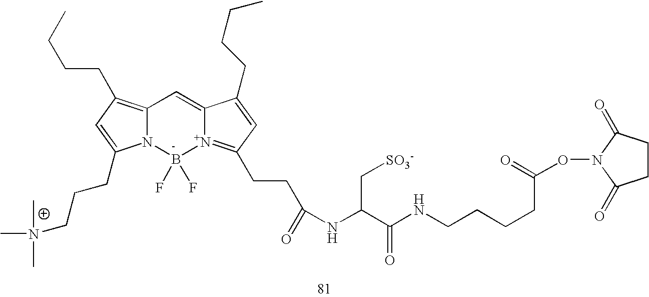

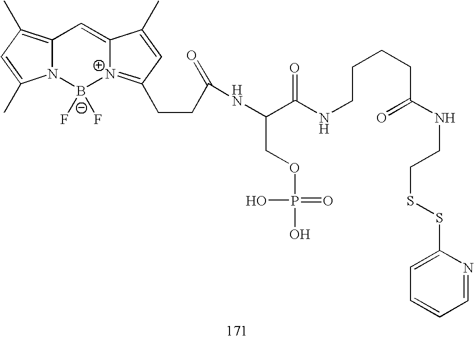

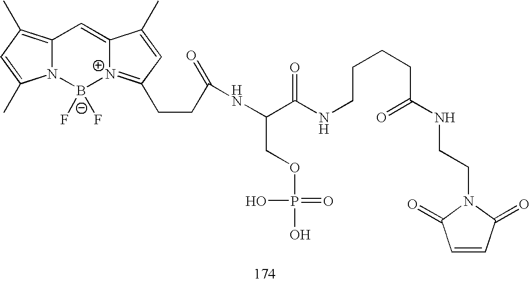

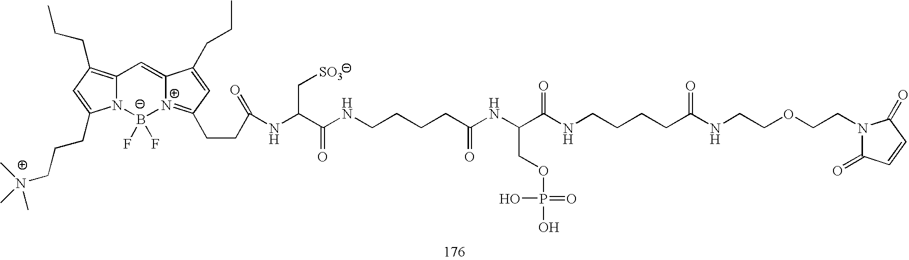

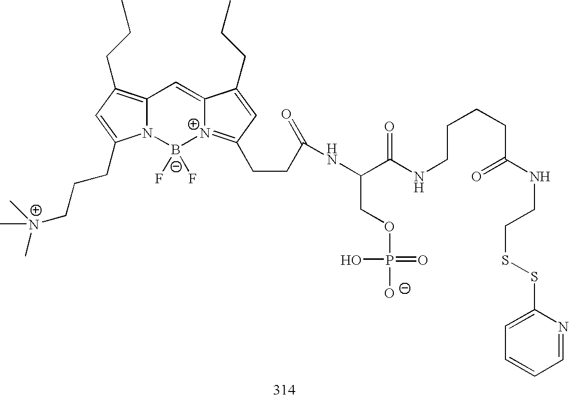

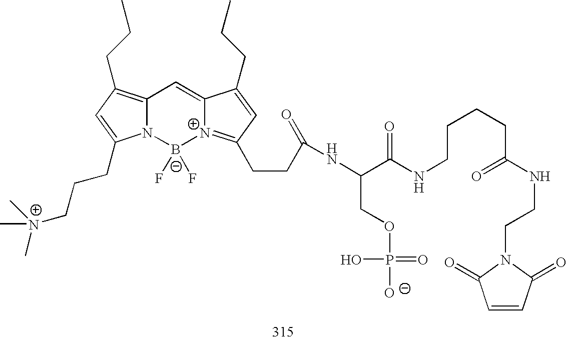

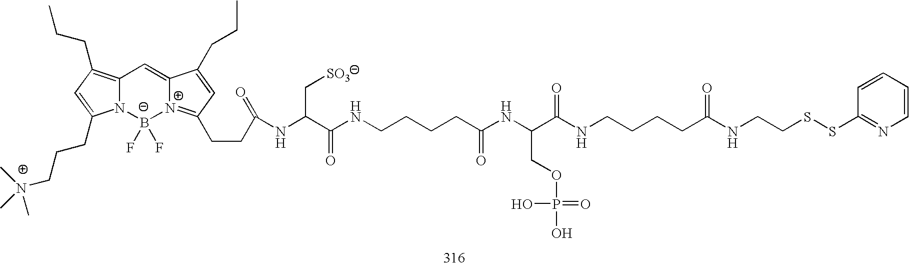

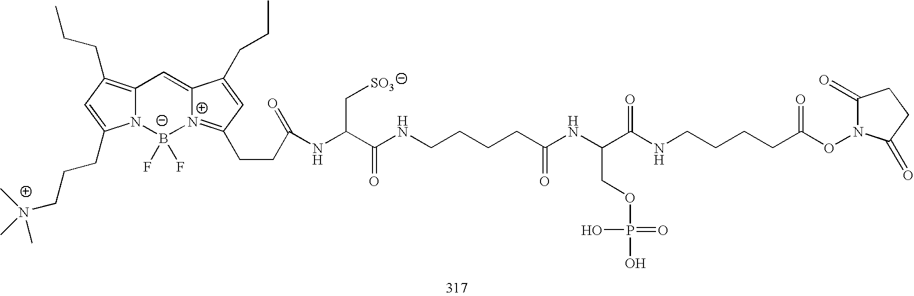

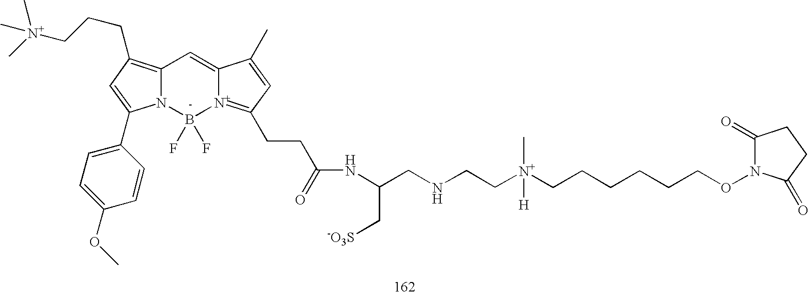

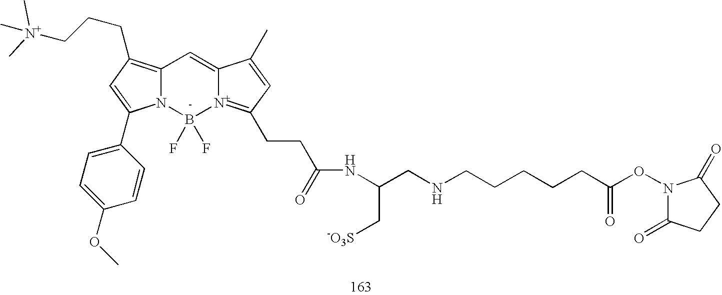

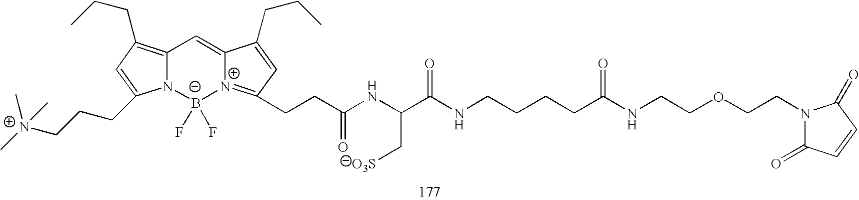

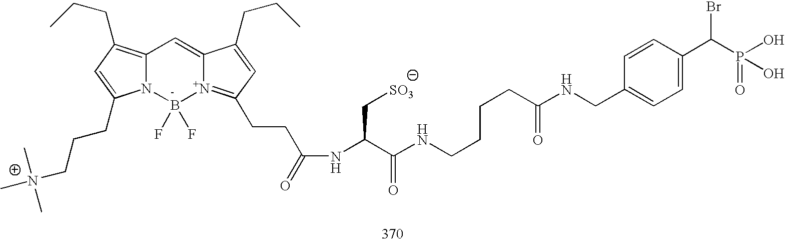

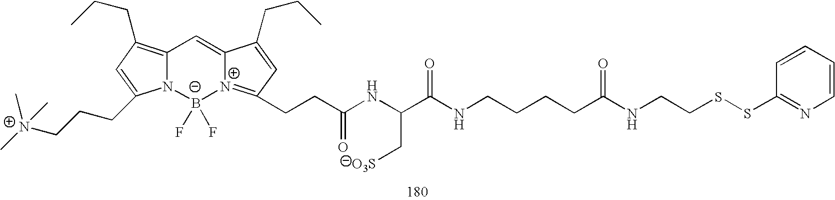

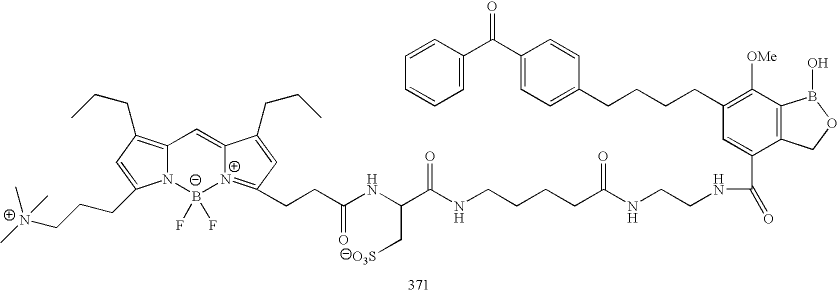

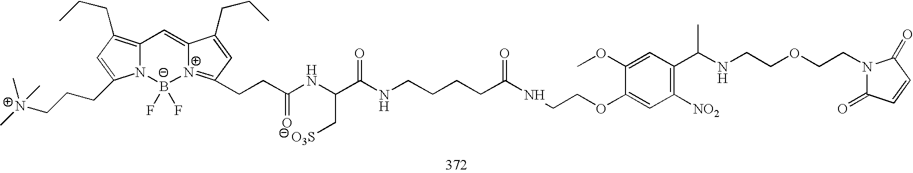

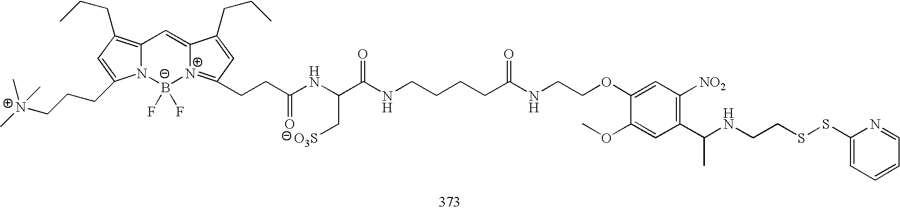

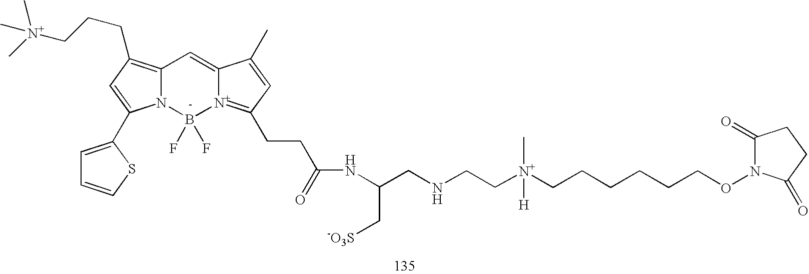

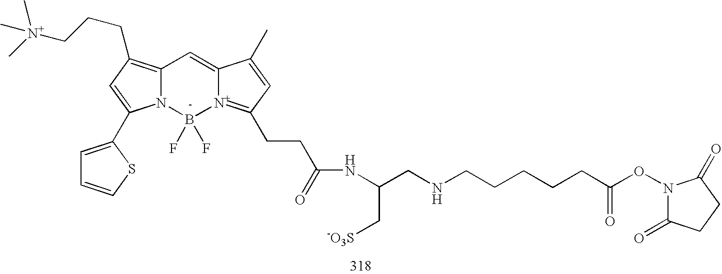

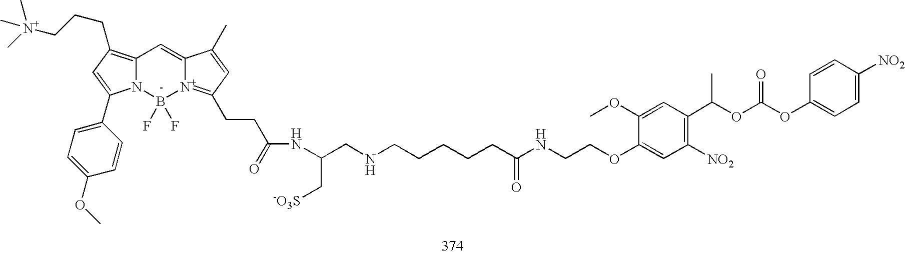

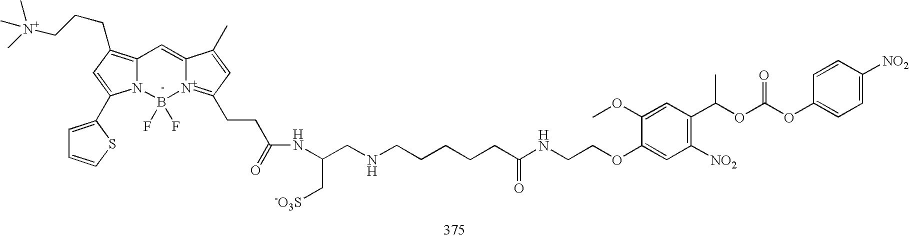

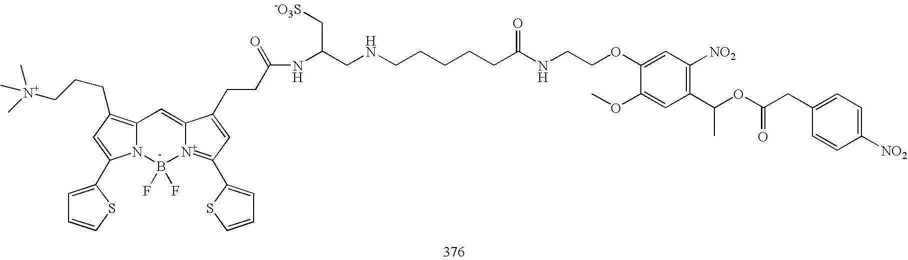

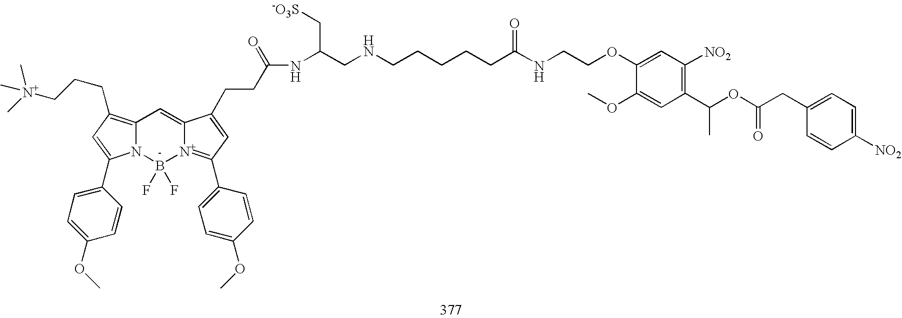

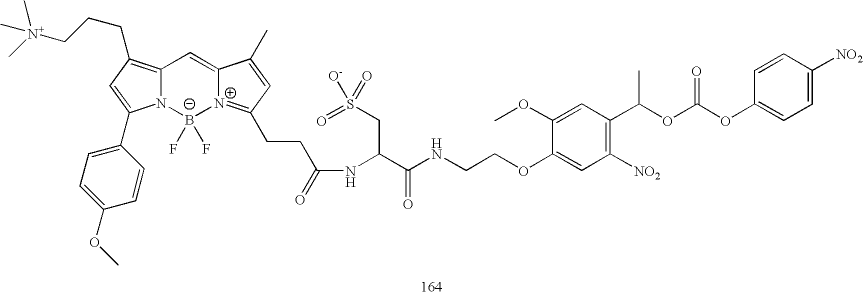

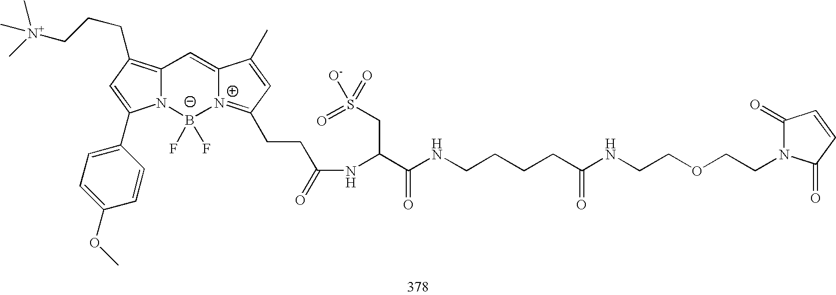

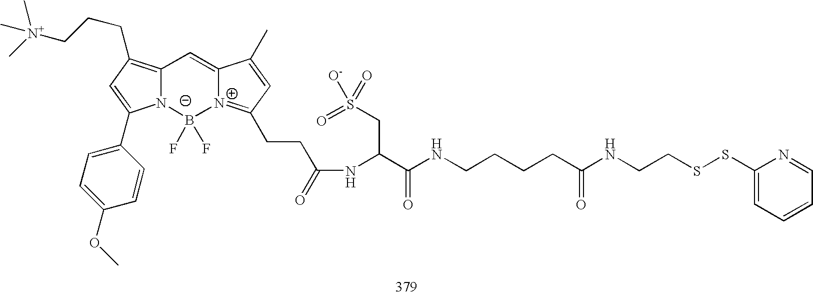

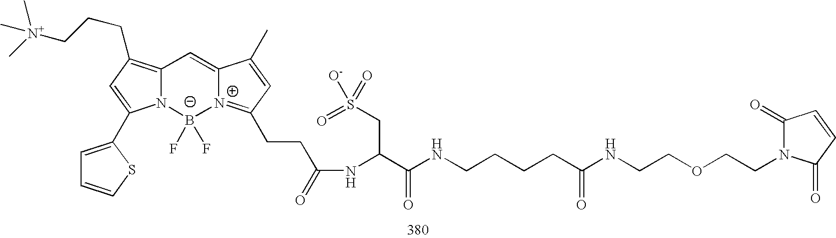

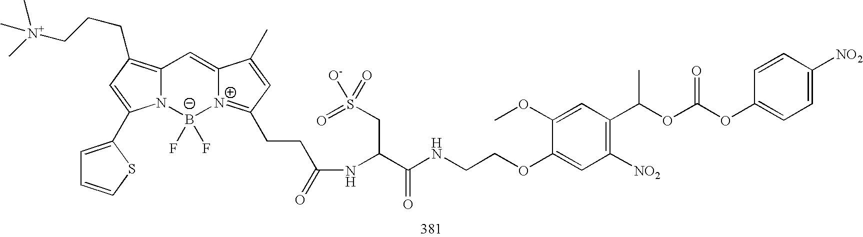

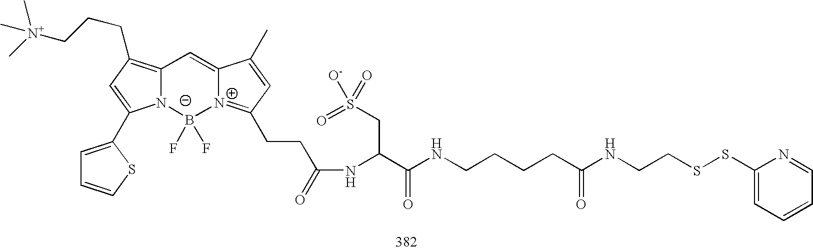

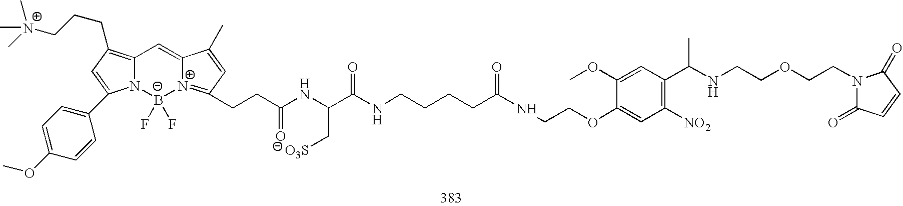

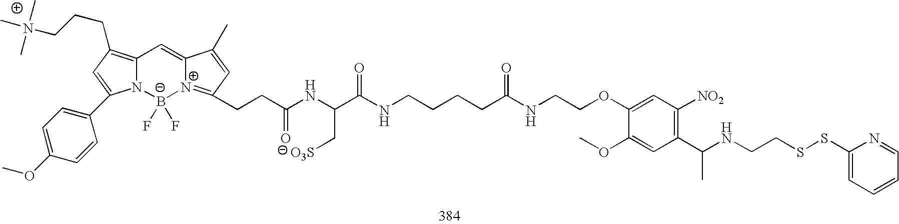

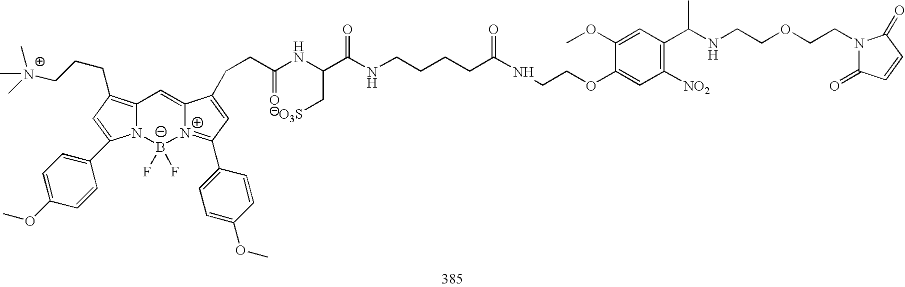

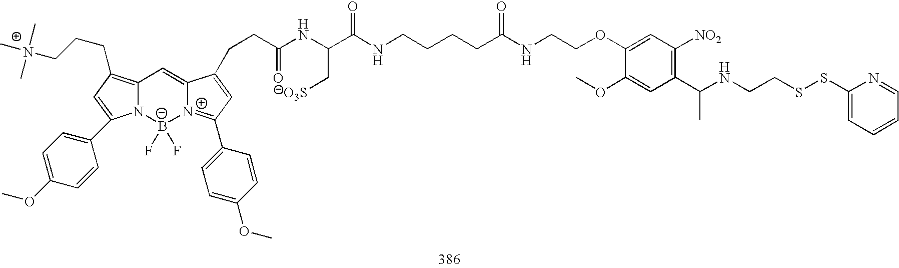

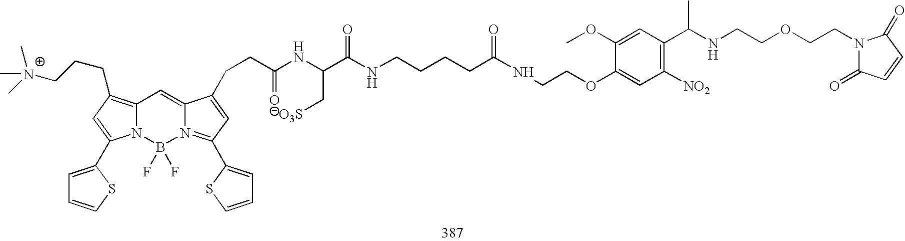

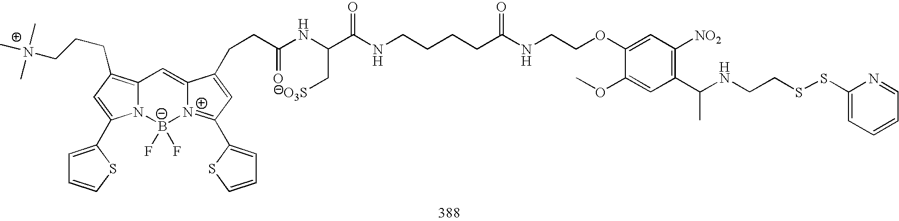

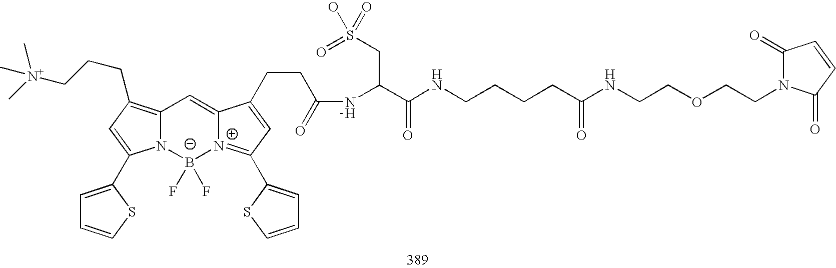

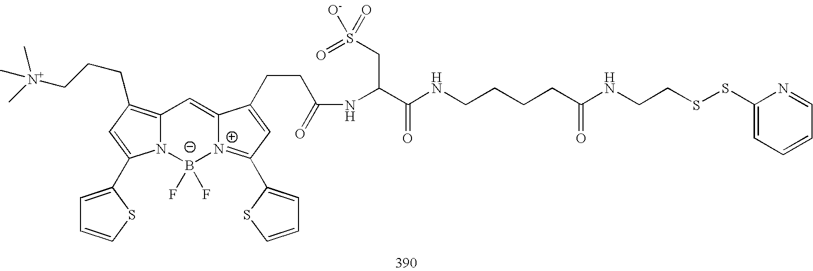

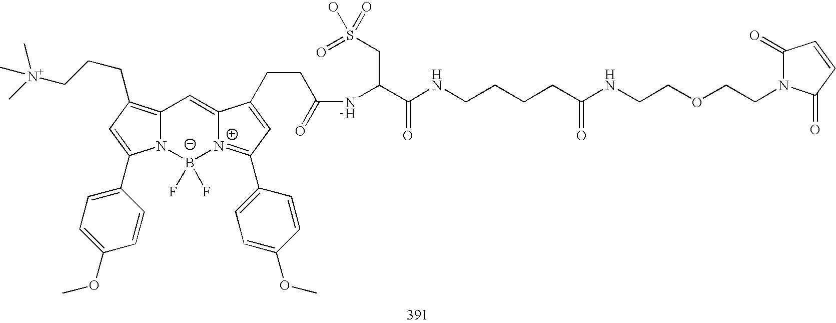

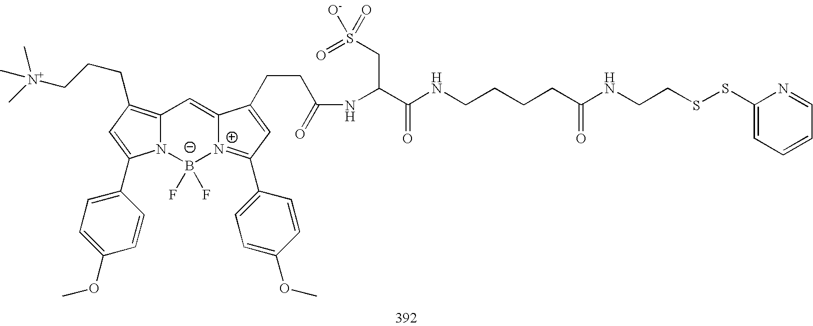

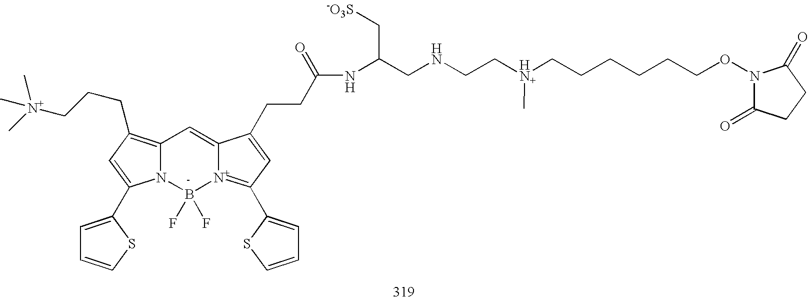

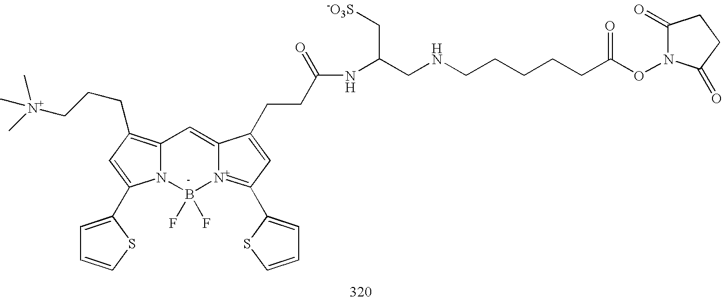

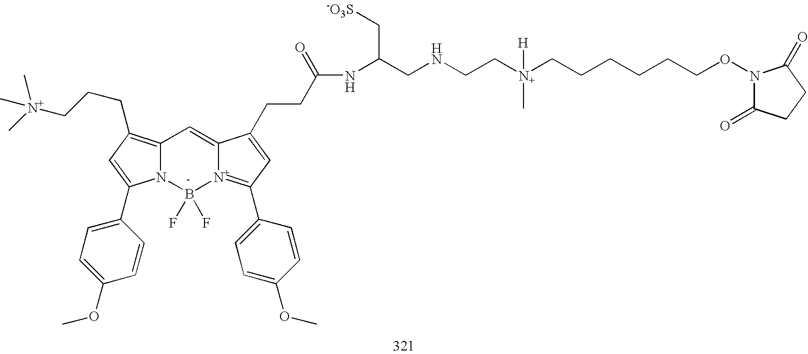

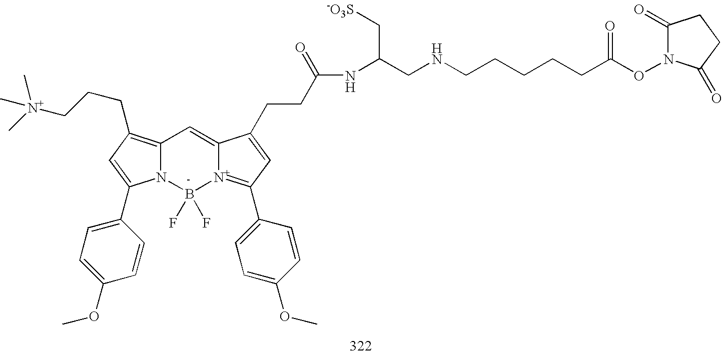

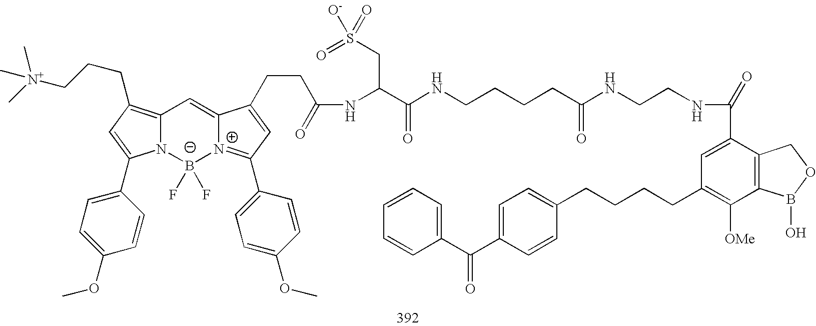

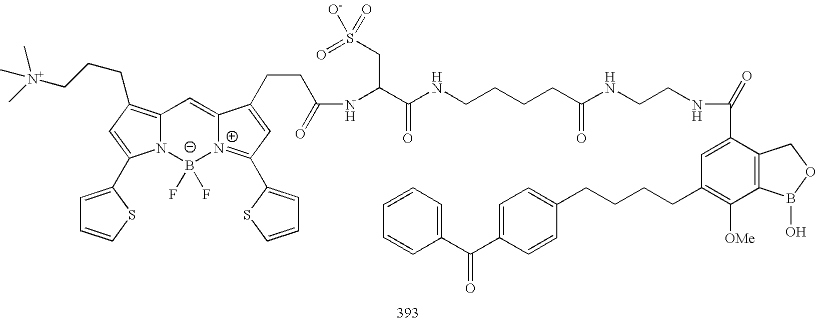

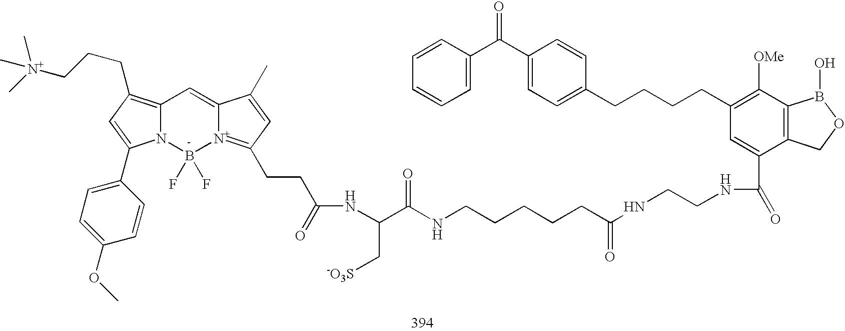

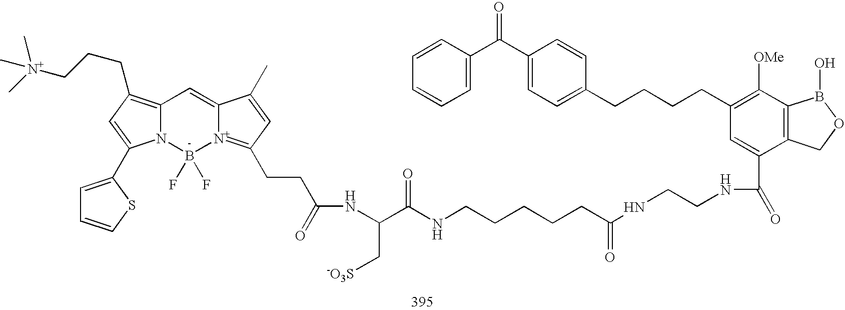

In some embodiments, an optical labeling molecule of structural Formula (II), or a salt or solvate thereof, is provided:

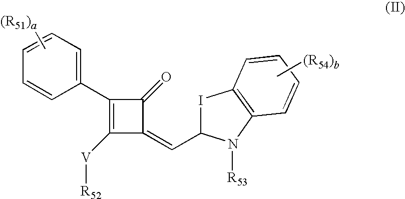

wherein:

a and b are independently 0, 1, 2, 3 or 4;

each R₅₁ and each R₅₄ are independently acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —S(O)ₜR₂₀, —SO₃H,

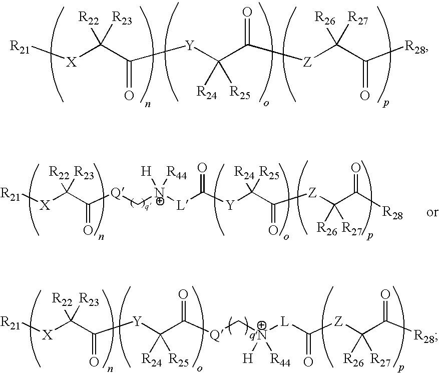

R₅₂ and R₅₃ are independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

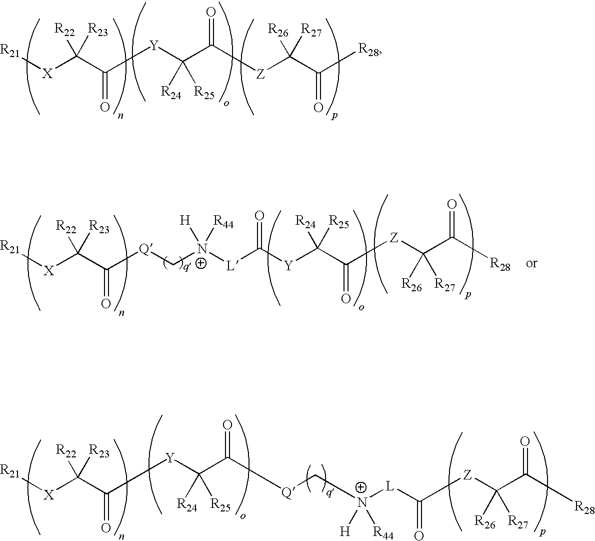

### I is —C(R₅₅R₅₆)—, —S—, —O— or —Se—;

R₅₅ and R₅₆ are independently hydrogen or alkyl;

### V is —NR₅₇ or —O—;

R₅₇ is hydrogen, alkyl or substituted alkyl or alternatively, R₅₂ and R₅₇ along with the nitrogen atom to which they are attached form a cycloheteroalkyl or substituted cycloheteroalkyl ring; and
R₂₀-R₂₉, R₄₃, R₄₄, n, o, p, q′, t, e, Q′, X, Y, Z and L′ are the same as defined above;
provided that:
(a) one and only one of R₅₁, R₅₂, R₅₃ or R₅₄ is

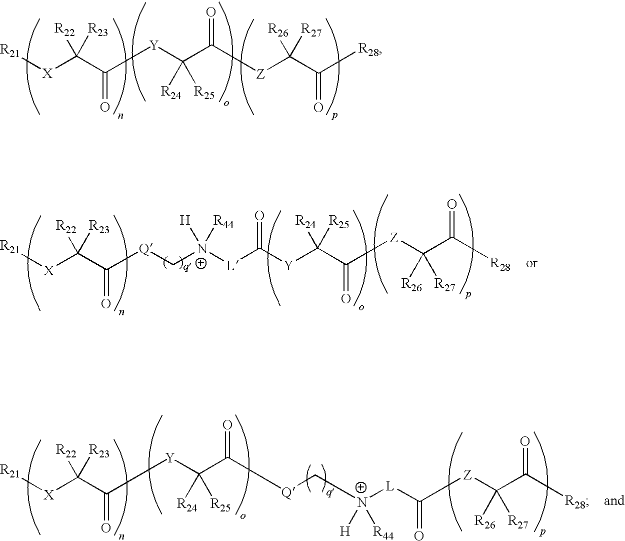

(b) R₅₁ to R₅₅ and R₆₀ contains at least one zwitterionic pair.

In some embodiments, R₅₁ and R₅₄ are acyl, alkyl, substituted alkyl, alkoxy, heteroalkyl, substituted heteroalkyl, halo, nitro, —S(O)ₜR₂₀, —SO₃H or

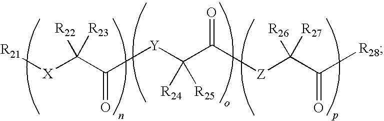

and R₅₂ and R₅₃ are acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃ or

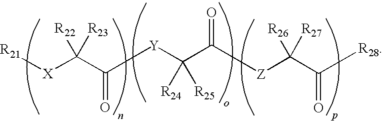

In other embodiments, R₅₁ and R₅₄ are acyl, alkyl, substituted alkyl, alkoxy, heteroalkyl, substituted heteroalkyl, halo, nitro, —S(O)ₜR₂₀, —SO₃H and R₅₂ and R₅₃ are acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃ or

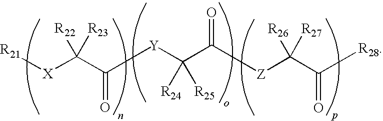

In still other embodiments, R₅₁ and R₅₄ are acyl, alkyl, substituted alkyl, alkoxy, heteroalkyl, substituted heteroalkyl, halo, nitro, —S(O)ₜR₂₀, —SO₃H and R₅₂ is acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃ and R₅₃ is

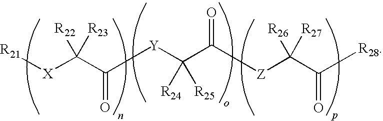

In still other embodiments, R₅₁ and R₅₄ are methoxy or —SO₃H. In still other embodiments, V is —O— and I is —(CR₅₅R₅₆)—. In still other embodiments, R₅₅ and R₅₆ are —CH₃. In still other embodiments, R₅, and R₅₄ are acyl, alkyl, substituted alkyl, alkoxy, heteroalkyl, substituted heteroalkyl, halo, nitro, —S(O)ₜR₂₀, —SO₃H and R₅₂ is acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃, R₅₃ is

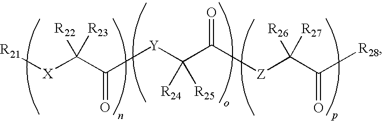

V is —O— and I is —(CR₅₅R₅₆)—. In still other embodiments, R₅₁ and R₅₄ are methoxy or —SO₃H, V is —O—, I is —(CR₅₅R₅₆)—, R₅₅ and R₅₆ are methyl, and R₅₃ is

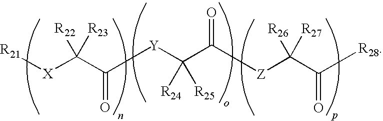

In some embodiments, the compounds of Formula (II) include the compounds of Table 2.

TABLE 2

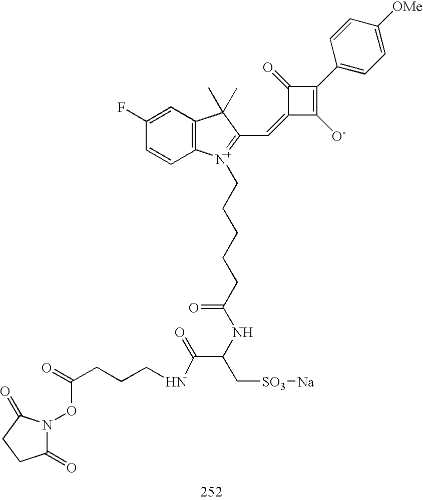

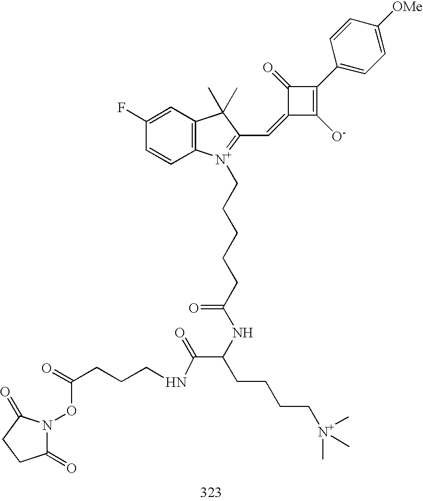

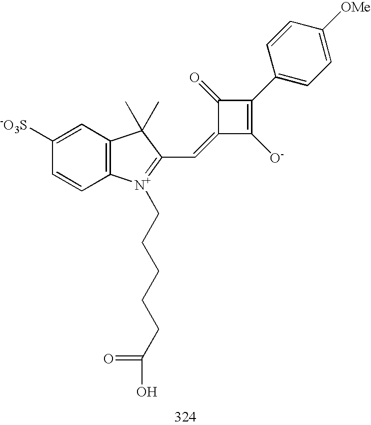

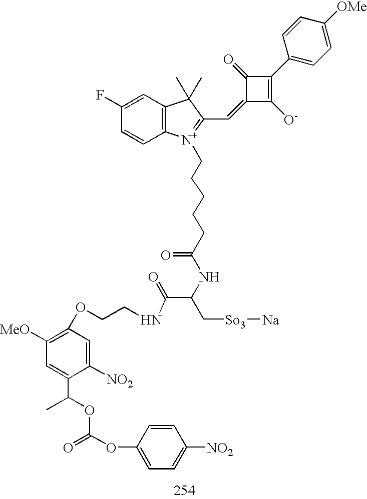

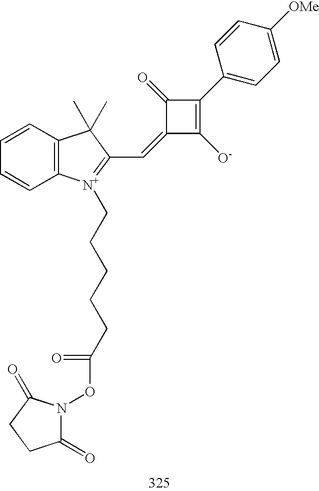

In some embodiments, an optical labeling molecule of structural Formula (III), or a salt or solvate thereof, is provided:

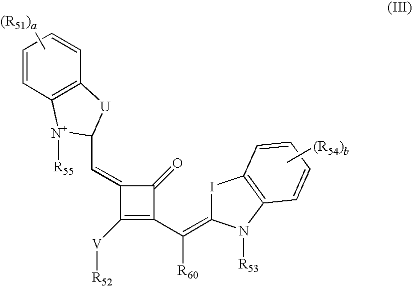

Wherein the following definitions apply for formula (III):
a and b are independently 0, 1, 2, 3 or 4;
R₅₅ is hydrogen, acyl, substituted acyl, alkoxycarbonyl, substituted alkoxycarbonyl, alkyl, substituted alkyl, aryl, substituted aryl, arylalkyl, substituted arylalkyl, aryloxycarbonyl, substituted aryloxycarbonyl, heteroaryl, substituted heteroaryl, heteroarylalkyl, substituted heteroarylalkyl, heteroalkyl, substituted heteroalkyl, —S(O)ᵢR₄₃,

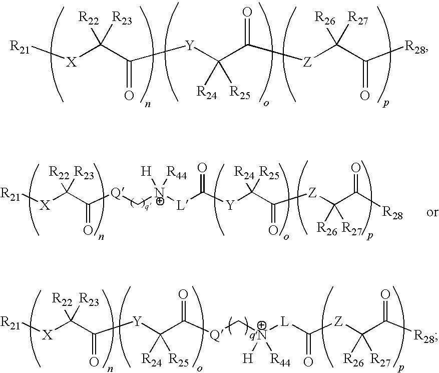

### I is —C(R₅₆R₅₇)—, —S—, —O— or —Se—;

### U is —C(R₅₈R₅₉)—, —S—, —O— or —Se—;

R₅₆, R₅₇, R₅₈ and R₅₉ are independently hydrogen or alkyl;
R₆₀ is hydrogen or alternatively R₆₀ and R₅₃ together with the atoms to which they are bonded form a cycloheteroalkyl or substituted cycloheteroalkyl ring;

### V is —NR₆₁, —O—,

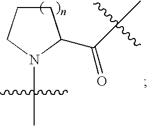

R₆, is hydrogen, alkyl or substituted alkyl or alternatively R₆₁ and R₅₂ along with the atoms to which they are bonded form a cycloheteroalkyl or substituted cycloheteroalkyl ring;
R₂₀-R₂₉, R₄₃, R₄₄, n, o, p, q′, t, e, Q′, X, Y, Z, L′, R₅₁-R₅₄;
provided that:
(a) one and only one of R₅₁, R₅₂, R₅₃, R₅₄ or R₅₅ is

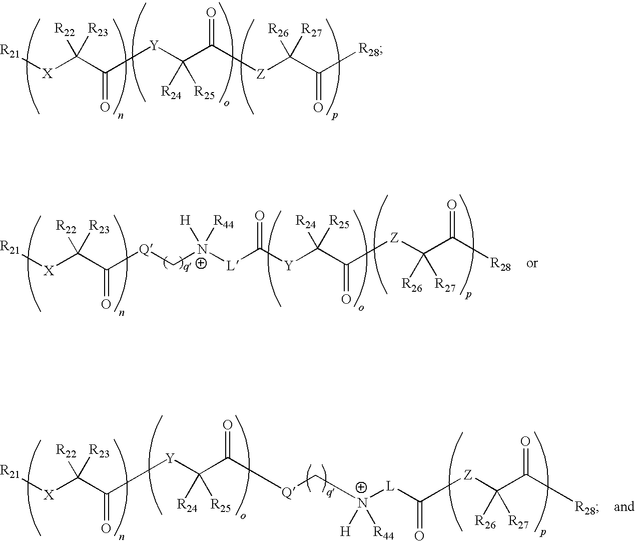

(b) R₅₁ to R₅₅ and R₆₀ contains at least one zwitterionic pair.

In other embodiments, R₅₁ and R₅₄ are each independently acyl, alkyl, substituted alkyl, alkoxy, heteroalkyl, substituted heteroalkyl, halo, nitro, —S(O)ₜR₂₀, —SO₃H or

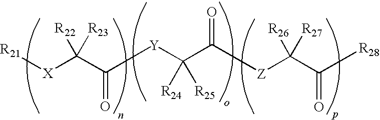

and R₅₂, R₅₃ and R₅₅ are each independently acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃ or

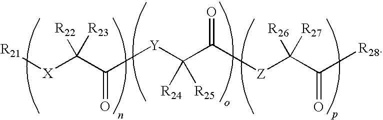

In still other embodiments, R₅₁ and R₅₄ are acyl, alkyl, substituted alkyl, alkoxy, heteroalkyl, substituted heteroalkyl, halo, nitro, —S(O)ₜR₂₀, —SO₃H and R₅₂, R₅₃ and R₅₅ are acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃ or

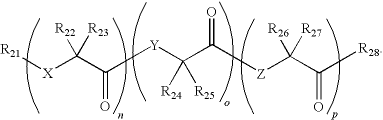

In still other embodiments, R₅₁ and R₅₄ are acyl, alkyl, substituted alkyl, alkoxy, heteroalkyl, substituted heteroalkyl, halo, nitro, —S(O)ₜR₂₀, —SO₃H and R₅₃ and R₅₅ are independently the same or different acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃ and R₅₂ is

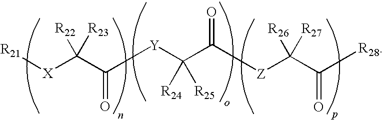

In still other embodiments, R₅₁ and R₅₄ are methoxy or —SO₃H. In many of the above embodiments, V is —NH—,

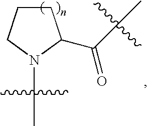

or R₆₁ and R₅₂ form a cycloheteroalkyl ring, I is —(CR₅₆R₅₇)— or S and U is —C(R₅₈R₅₉)—. In still other embodiments, R₅₆, R₅₇, R₅₈ and R₅₉ are —CH₃. In still other embodiments, R₅₁ and R₅₄ are acyl, alkyl, substituted alkyl, alkoxy, heteroalkyl, substituted heteroalkyl, halo, nitro, —S(O)ₜR₂₀, —SO₃H and R₅₃ and R₅₅ are acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃, R₅₂ is

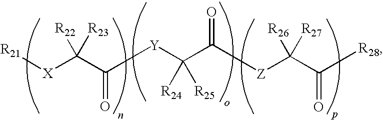

### V is —NH— or

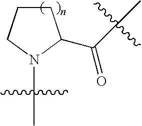

### and I is —(CR₅₅R₅₆)—.

In still other embodiments, R₅₁ and R₅₄ are —SO₃H, V is —NH—,

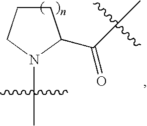

or R₆₁ and R₅₂ form a cycloheteroalkyl ring, I is —(CR₅₆R₅₇)— or —S—, U is —C(R₅₈R₅₉), R₅₆, R₅₇, R₅₈ and R₅₉ are methyl, R₅₃ and R₅₅ are methyl or —(CH₂)₄N⁺(CH₃)₃ and R₅₂ is

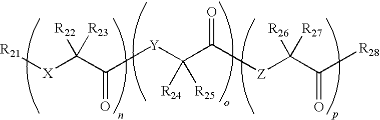

In some embodiments, the compounds of Formula (III) include the compounds of Table 3.

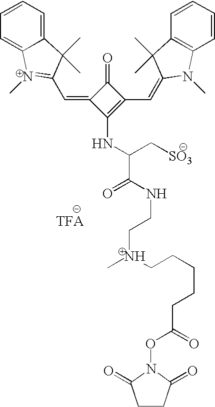

223;

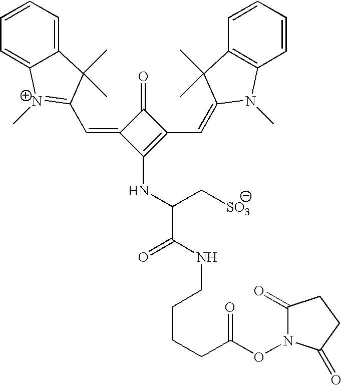

225

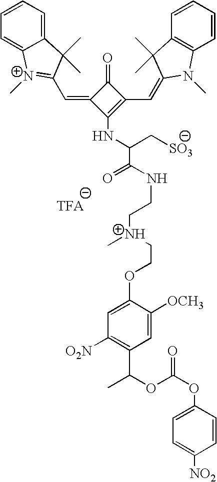

228

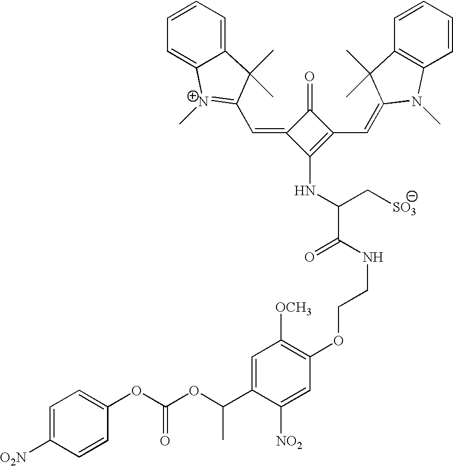

230

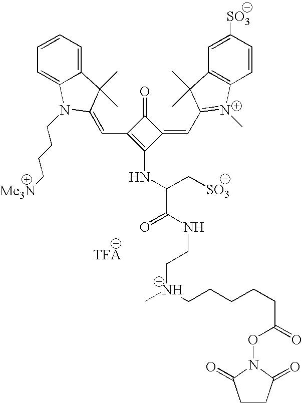

236

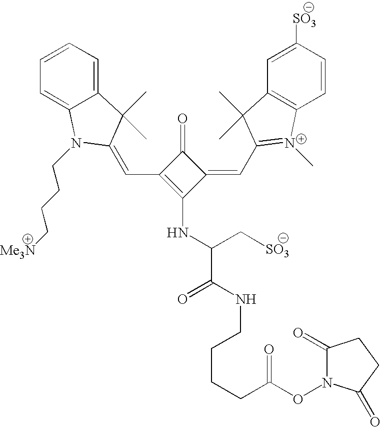

238

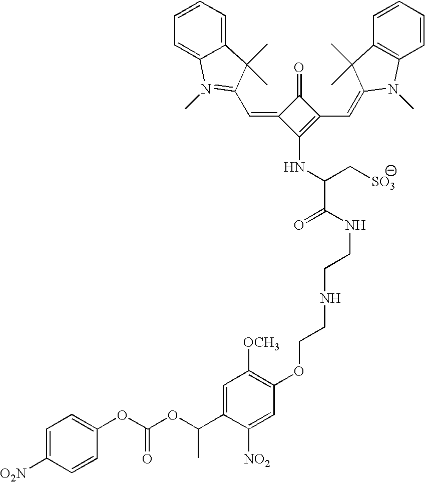

326

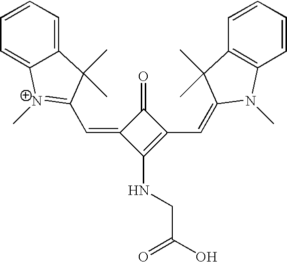

327

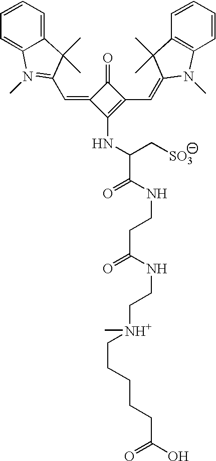

222

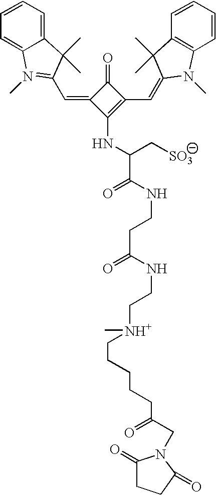

223

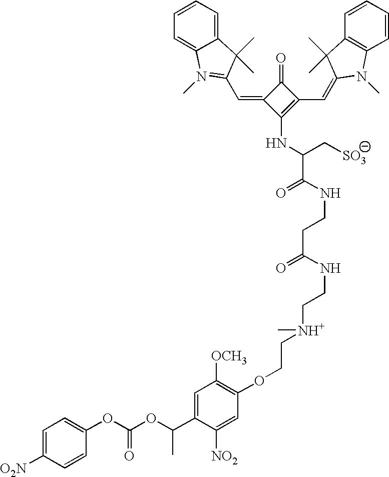

228

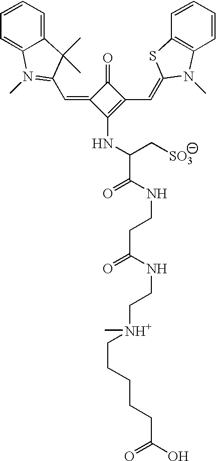

328

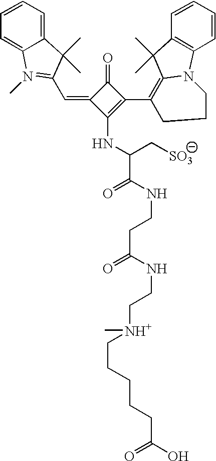

329

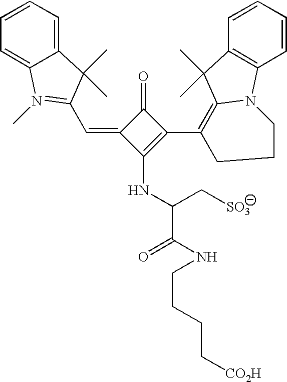

330

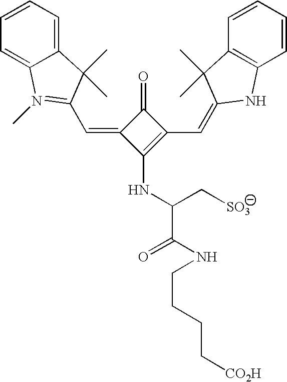

331

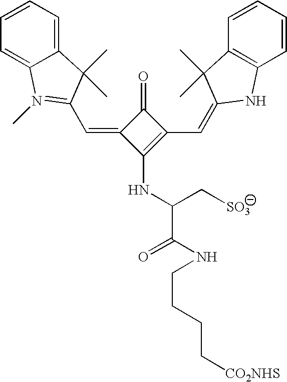

332

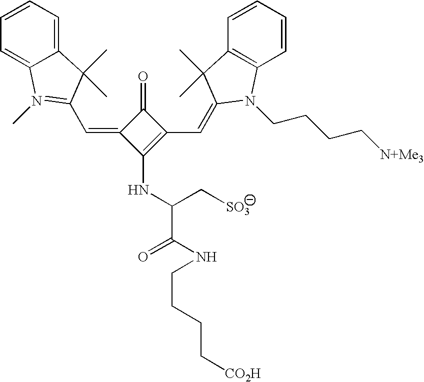

333

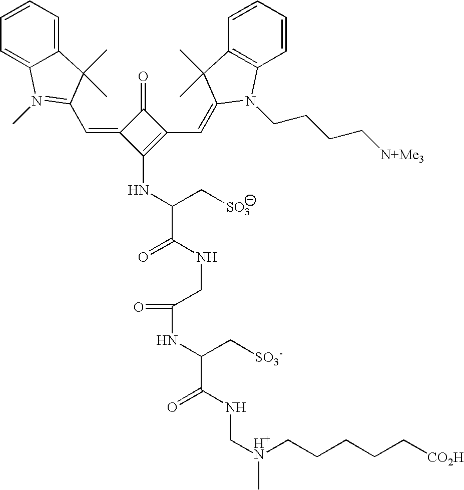

334

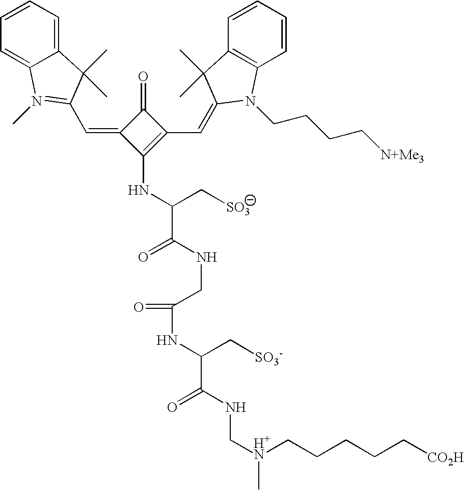

335

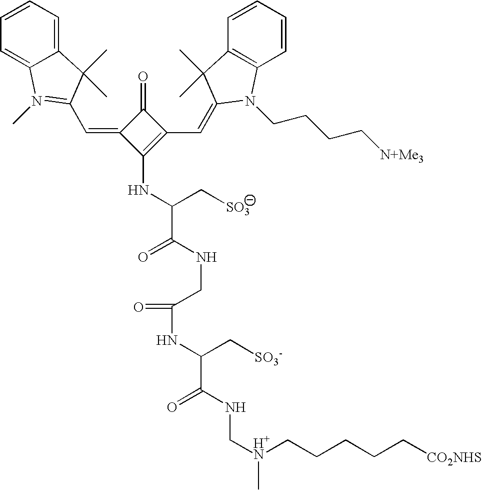

336

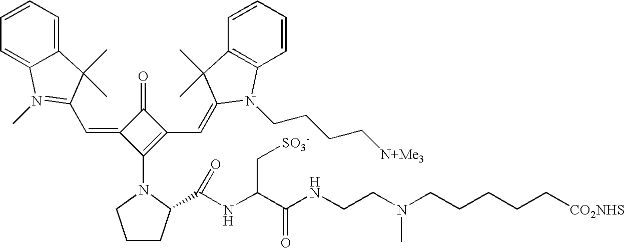

337

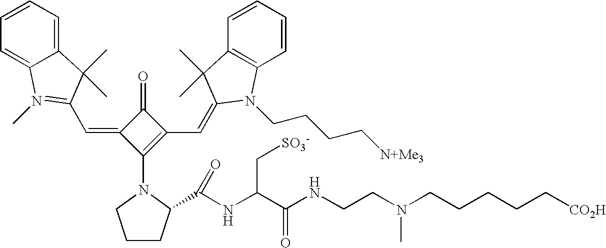

338

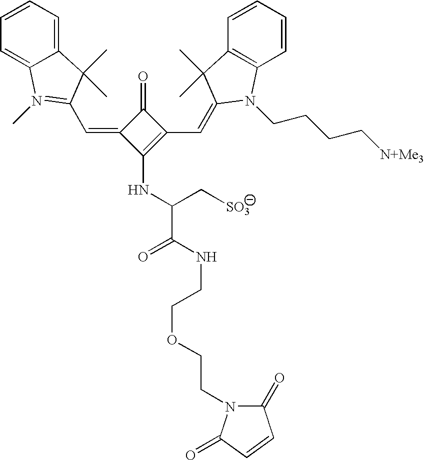

339

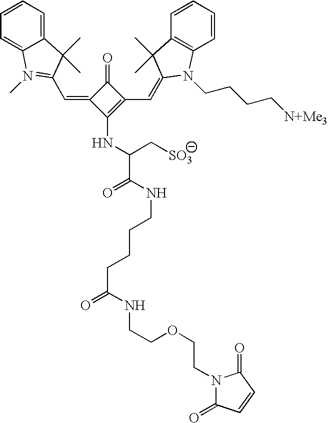

340

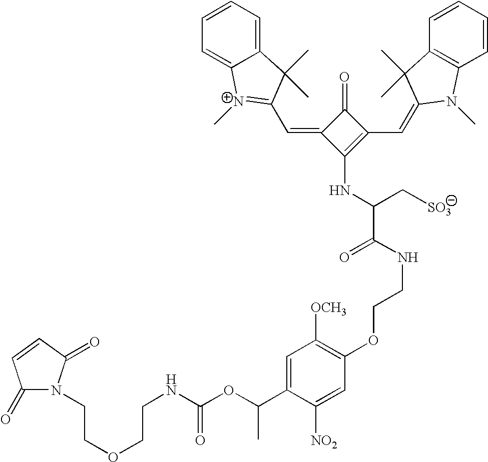

341

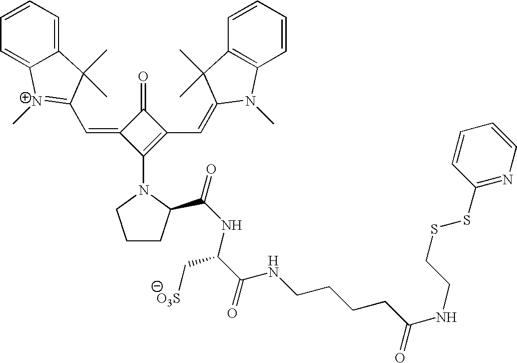

242

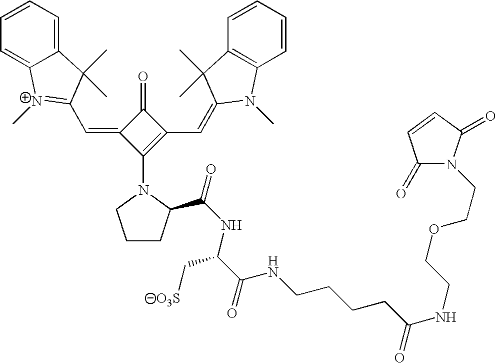

241

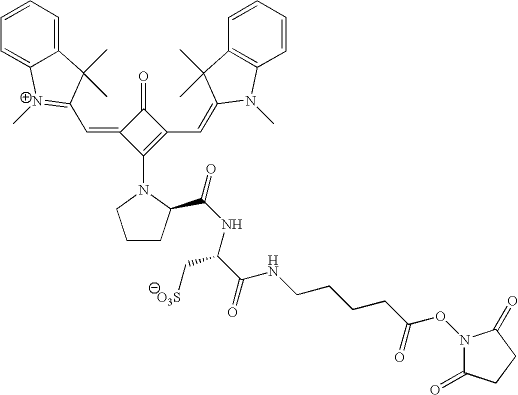

240

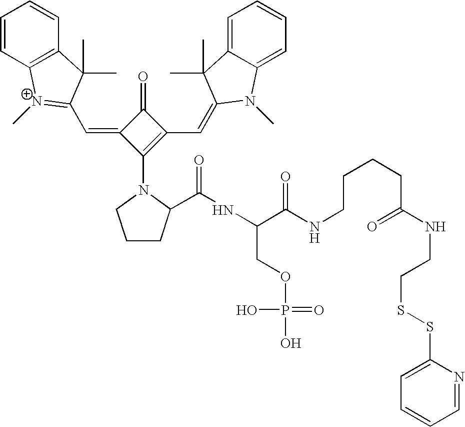

342

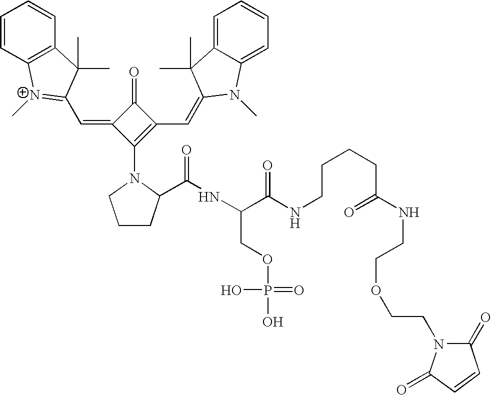

244

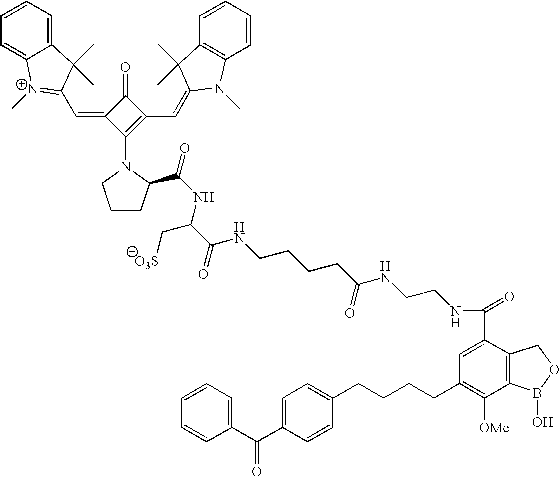

396

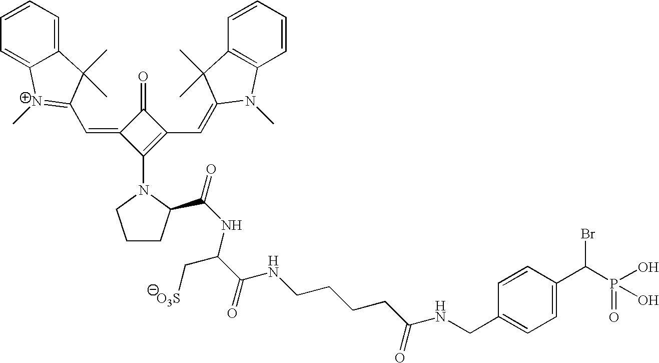

397

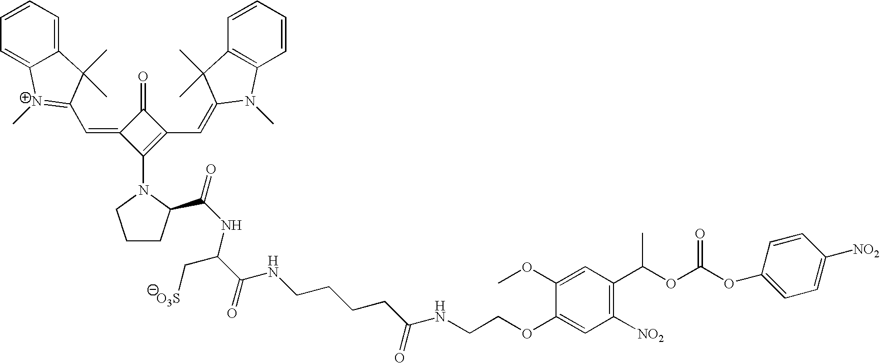

398

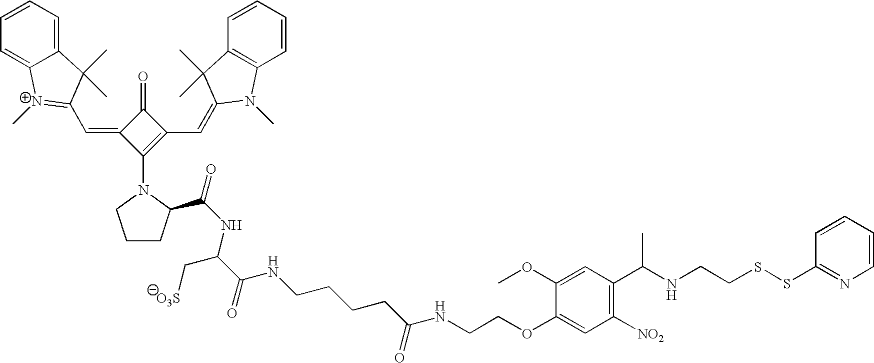

399

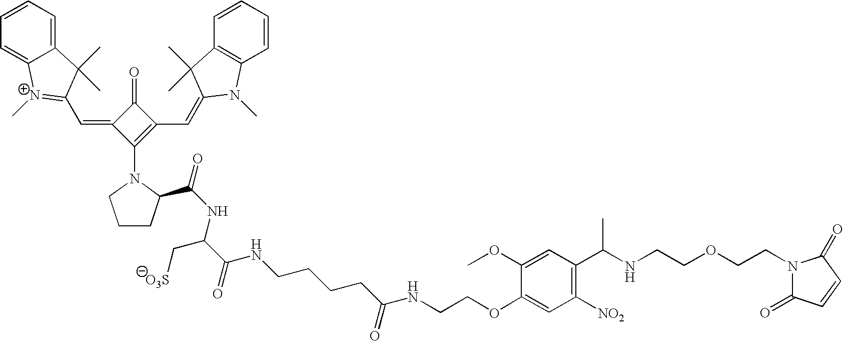

400

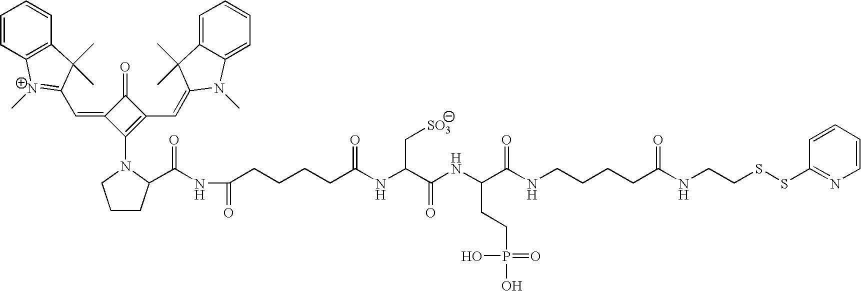

343

In some embodiments, an optical labeling molecule of structural Formula (IV), or a salt or solvate thereof, is provided:

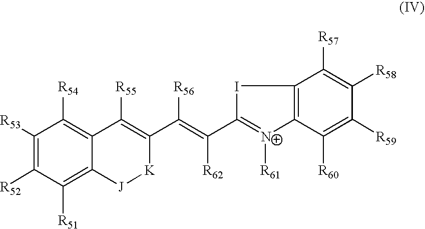

wherein:

### J is —O— or —NR₆₃;

R₆₃ is hydrogen, alkyl or substituted alkyl;

### K is —C(O)— or —C(S)—;

R₅₁-R₆₀ and R₆₂ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —S(O)ₜR₂₀, —SO₃H,

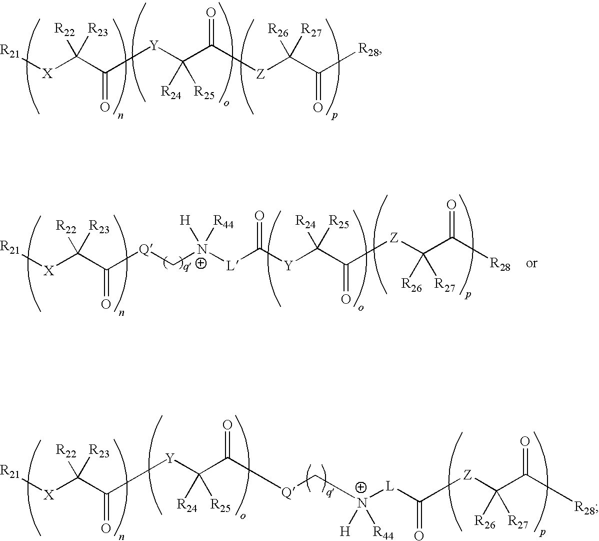

R₆₁ is independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

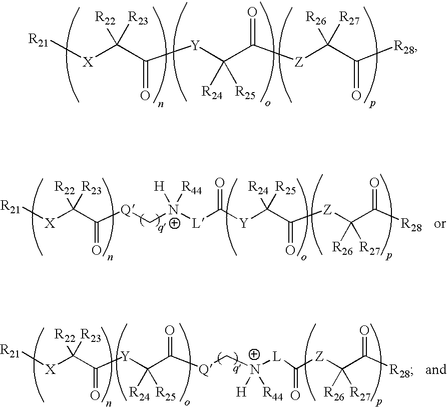

and R₂₀-R₂₉, R₄₃, R₄₄, n, o, p, q′, t, e, Q′, X, Y, Z, L′ and I are as previously defined;
provided that one and only one of R₅₀-R₆₂ is

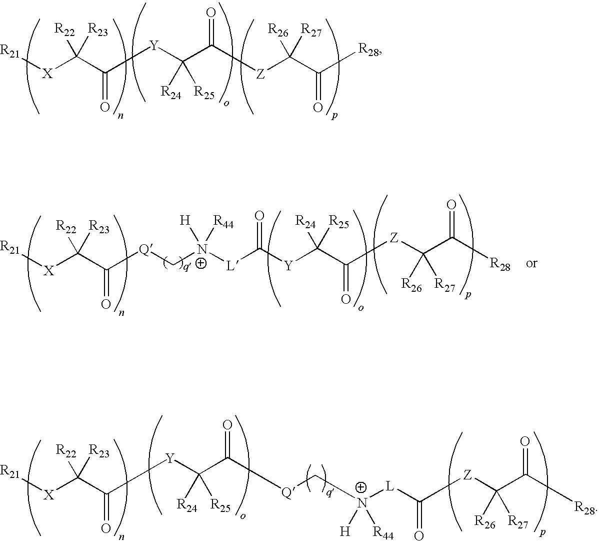

In some embodiments, a optical labeling molecule of structural Formula (V), or a salt or solvate thereof, is provided:

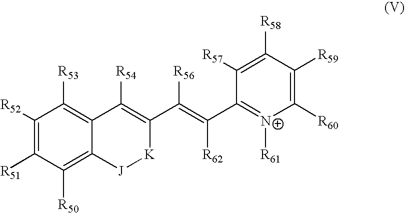

wherein:

### J is —O— or —NR₆₃;

R₆₃ is hydrogen, alkyl or substituted alkyl;

### K is —C(O)— or —C(S)—;

R₅₁-R₆₀ and R₆₂ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —S(O)ₜR₂₀, —SO₃H,

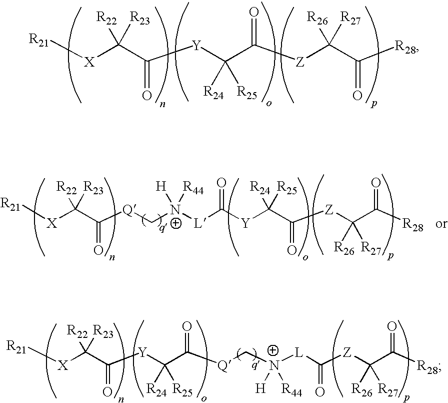

R₆₁ is independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

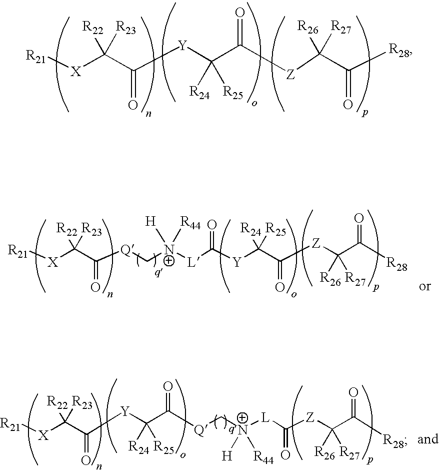

and R₂₀-R₂₉, R₄₃, R₄₄ n, o, p, q′, t, e, Q′, X, Y, Z and L′ are as previously defined;
provided that one and only one of R₅₀-R₆₂ is

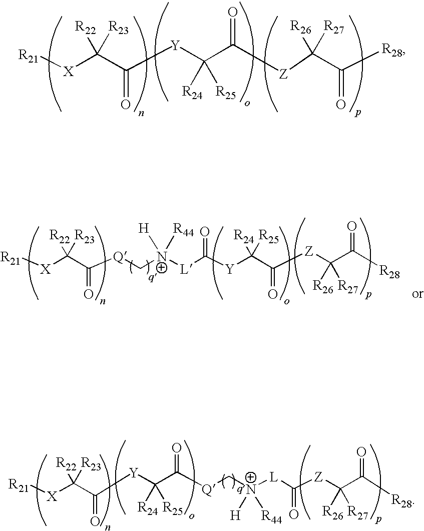

In some embodiments, a optical labeling molecule of structural Formula (VI), or a salt or solvate thereof, is provided:

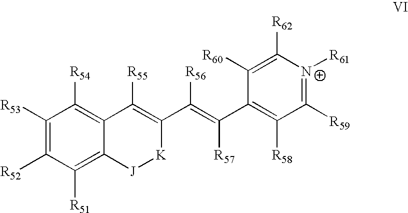

wherein:

### J is —O— or —NR₆₃;

R₆₃ is hydrogen, alkyl or substituted alkyl;

### K is —C(O)— or —C(S)—;

R₅₁-R₆₀ and R₆₂ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —S(O)ₜR₂₀, —SO₃H,

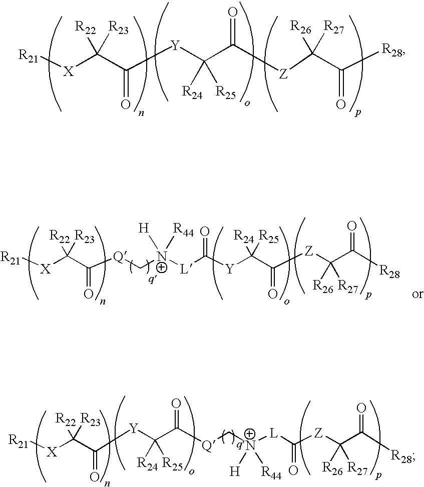

R₆₁ is independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

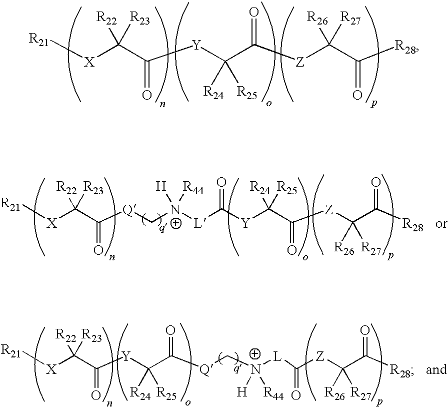

and R₂₀-R₂₉, R₄₃, R₄₄, n, o, p, q′, t, e, Q′, X, Y, Z and L′ are as previously defined;
provided that one and only one of R₅₀⁻R₆₂ is

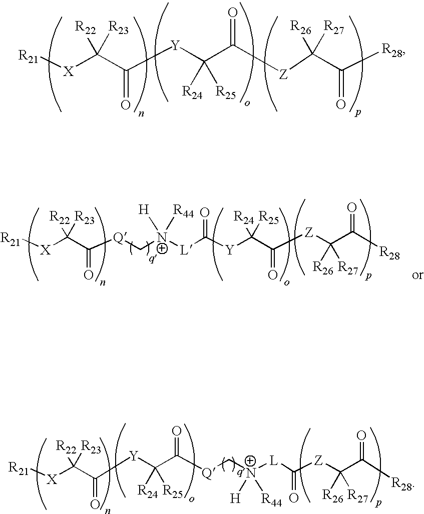

In some embodiments, an optical labeling molecule of structural Formula (VII), or a salt or solvate thereof, is provided:

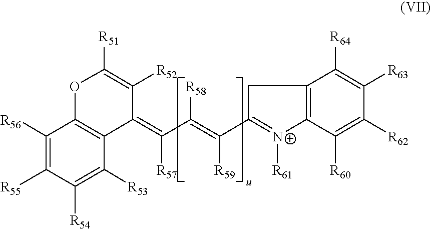

wherein:
u is 0, 1, 2, 3, 4, or 5;
R₅₁-R₆₀ and R₆₂-R₆₄ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —S(O)ₜR₂₀, —SO₃H,

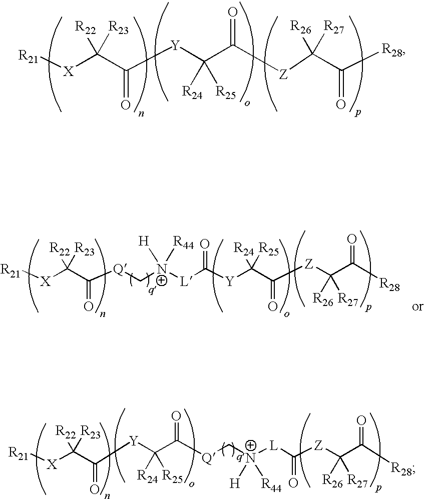

R₆₁ is independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

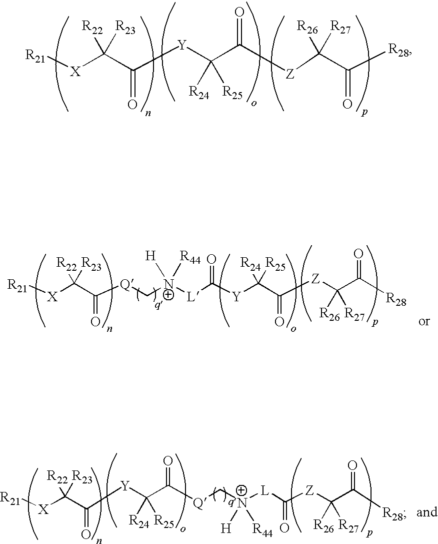

and R₂₀-R₂₉, R₄₃, R₄₄, n, o, p, q′, t, e, Q′, X, Y, Z, L′ and I are as previously defined;
provided that one and only one of R₅₀-R₆₄ is

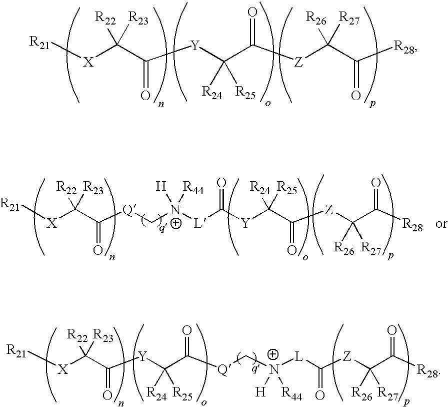

In some embodiments, an optical labeling molecule of structural Formula (VIII), or a salt or solvate thereof, is provided:

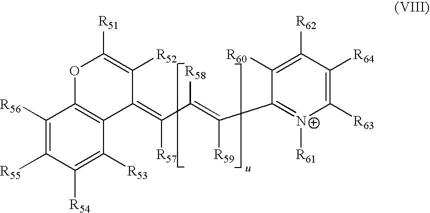

wherein:
u is 0, 1, 2, 3, 4, or 5;
R₅₁-R₆₀ and R₆₂-R₆₄ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —S(O)ₜR₂₀, —SO₃H,

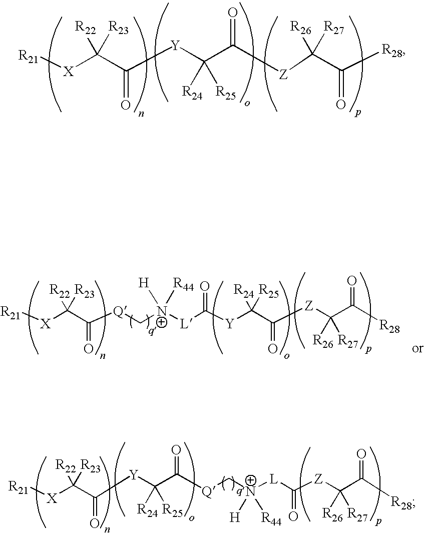

R₆₁ is independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

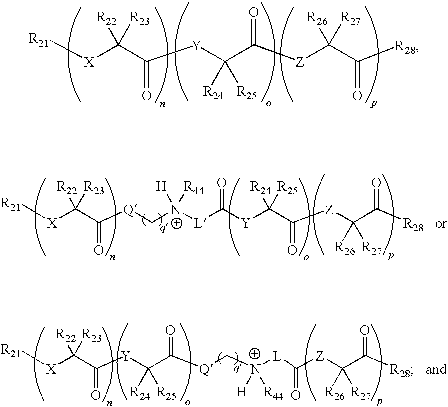

and R₂₀-R₂₉, R₄₃, R₄₄, n, o, p, q′, t, e, Q′, X, Y, Z and L′ are as previously defined;
provided that one and only one of R₅₀-R₆₄ is

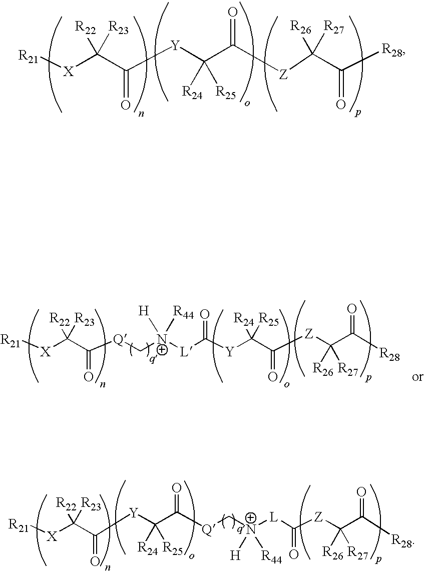

In some embodiments, an optical labeling molecule of structural Formula (IX), or a salt or solvate thereof, is provided:

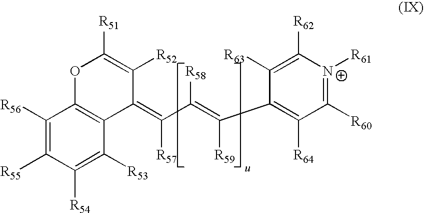

wherein:
u is 0, 1, 2, 3, 4, or 5;
R₅₁-R₆₀ and R₆₂-R₆₄ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —S(O)ₜR₂₀, —SO₃H,

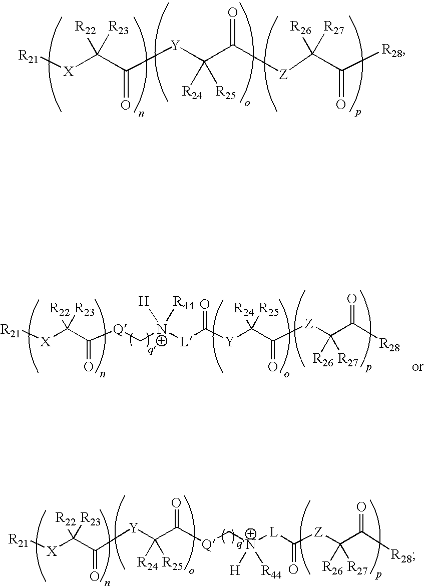

R₆₁ is independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

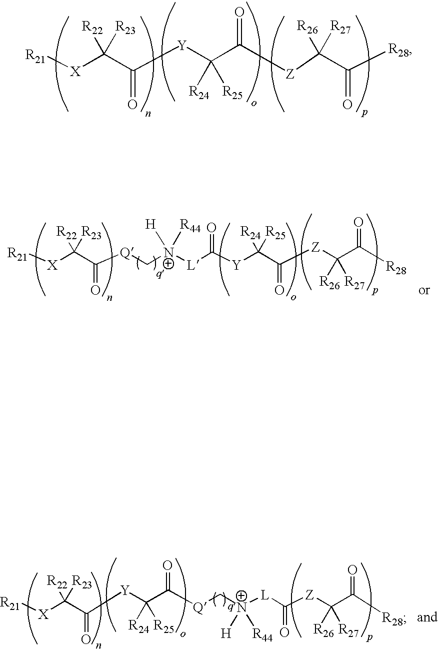

and R₂₀-R₂₉, R₄₃, R₄₄, n, o, p, q′, t, e, Q′, X, Y, Z and L′ are as previously defined;
provided that one and only one of R₅₀-R₆₄ is

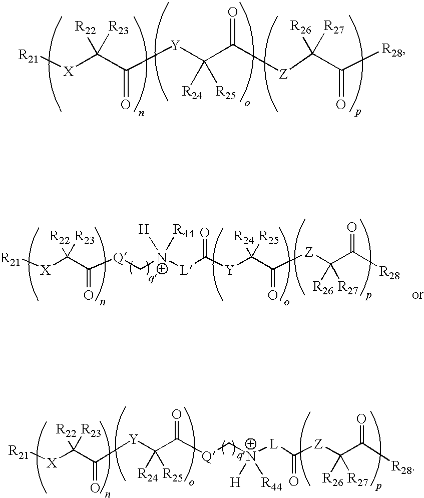

In some embodiments, an optical labeling molecule of structural Formula (X), or a salt or solvate thereof, is provided:

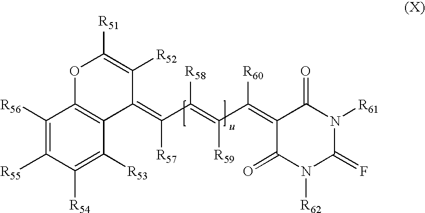

wherein:

### F is O or S;

u is 0, 1, 2, 3, 4, or 5;
R₅₁-R₆₀ and R₆₂ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —S(O)ₜR₂₀, —SO₃H,

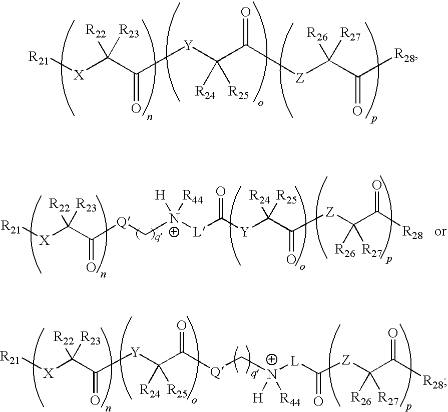

R₆₁ is independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

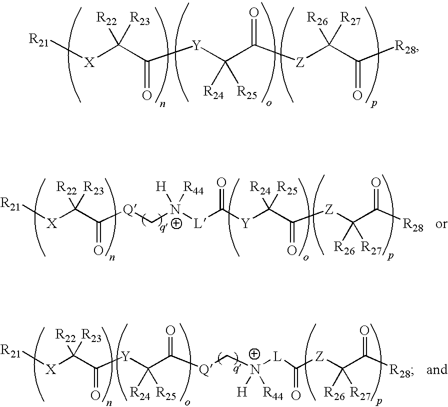

and R₂₀-R₂₉, R₄₃, R₄₄, n, o, p, q′, t, e, Q′, X, Y, Z and L′ are as previously defined;
provided that one and only one of R₅₀-R₆₂ is

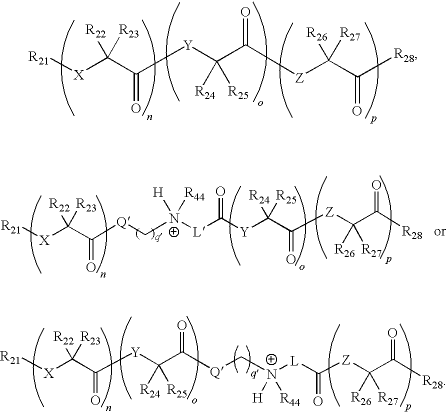

In some embodiments, an optical labeling molecule of structural Formula (XI), or a salt or solvate thereof, is provided:

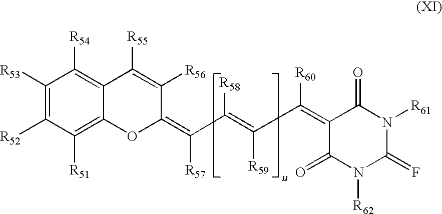

wherein:

### F is O or S;

u is 0, 1, 2, 3, 4, or 5;
R₅₁-R₆₀ and R₆₂ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —S(O)ₜR₂₀, —SO₃H,

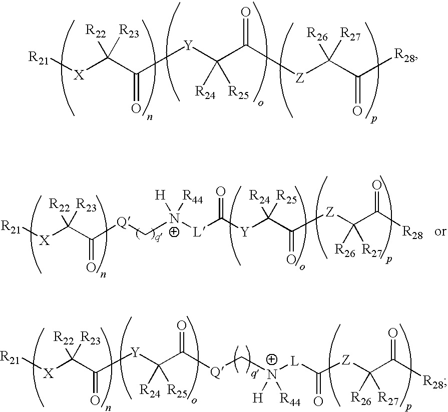

R₆₁ is independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

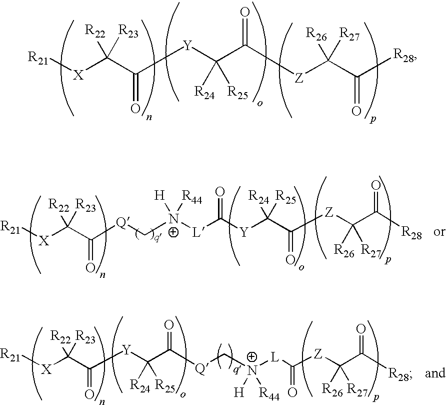

and R₂₀-R₂₉, R₄₃, R₄₄, n, o, p, q′, t, e, Q′, X, Y, Z and L′ are as previously defined;
provided that one and only one of R₅₀-R₆₂ is

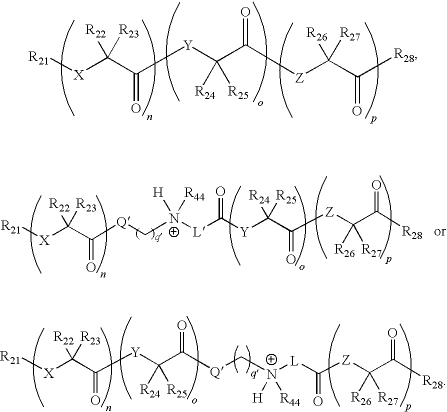

In some embodiments, a optical labeling molecule of structural Formula (XII), or a salt or solvate thereof is provided:

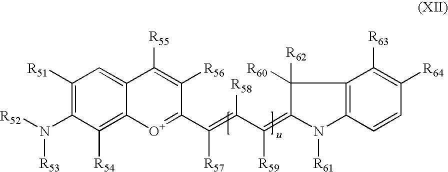

wherein:
u is 0, 1, 2, 3, 4, or 5;
R₅₁⁻R₆₀ and R₆₂-R₆₄ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl; heteroalkyl, substituted heteroalkyl, halo, nitro, —S(O)ₜR₂₀, —SO₃H,

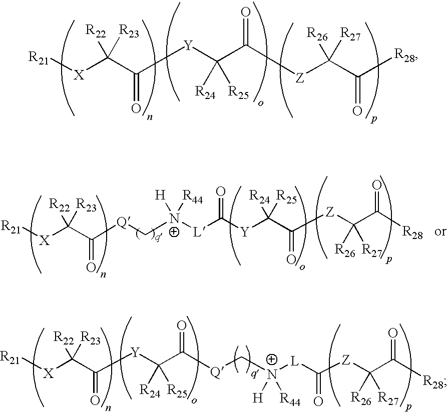

R₆₁ is independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

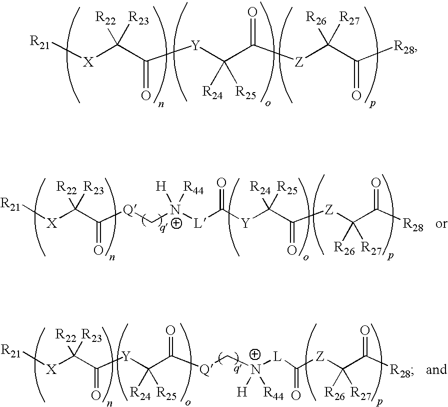

and R₂₀-R₂₉, R₄₃, R₄₄, n, o, p, q′, t, e, Q′, X, Y, Z and L′ are as previously defined;
provided that one and only one of R₅₀-R₆₄ is

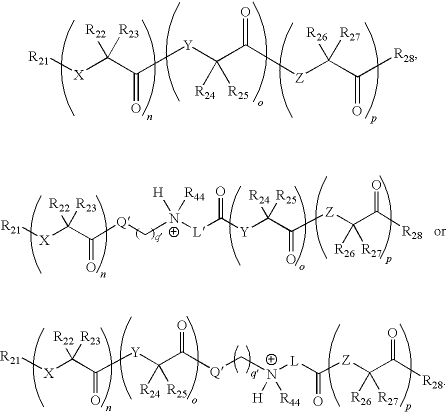

In some embodiments, a optical labeling molecule of structural Formula (XIII), or a salt or solvate thereof, is provided:

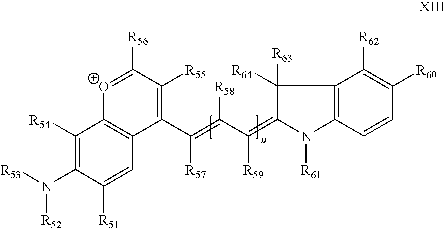

wherein:
u is 0, 1, 2, 3, 4, or 5;
R₅₁-R₆₀ and R₆₂-R₆₄ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —S(O)ₜR₂₀, —SO₃H,

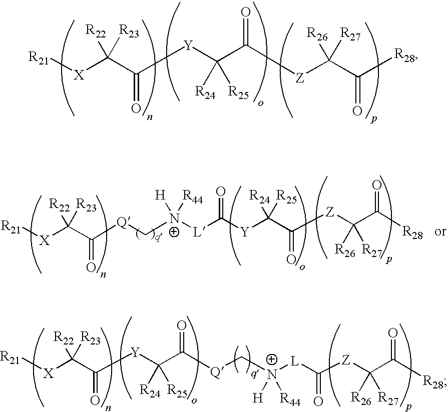

R₆₁ is independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

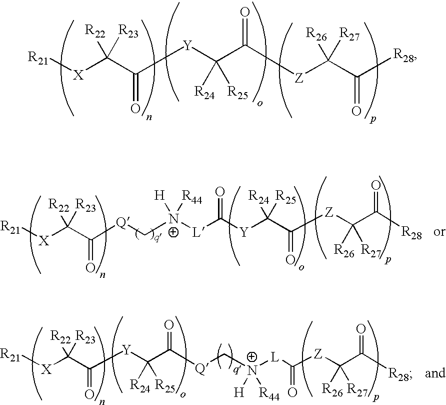

and R₂₀-R₂₉, R₄₃, R₄₄, n, o, p, q′, t, e, Q′, X, Y, Z and L′ are as previously defined;
provided that one and only one of R₅₀-R₆₄ is

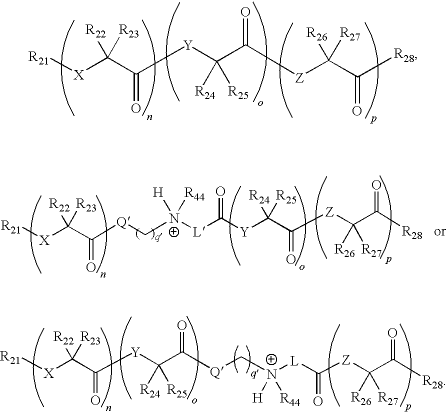

In some embodiments, an optical labeling molecule of structural Formula (XIV), or a salt or solvate thereof, is provided:

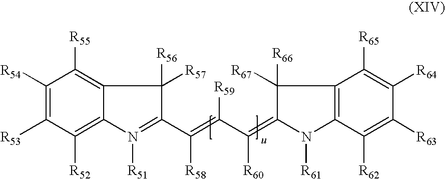

wherein:
u is 0, 1, 2, 3, 4, or 5;
R₅₂-R₆₀ and R₆₂-R₆₅ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —S(O)ₜR₂₀, —SO₃H,

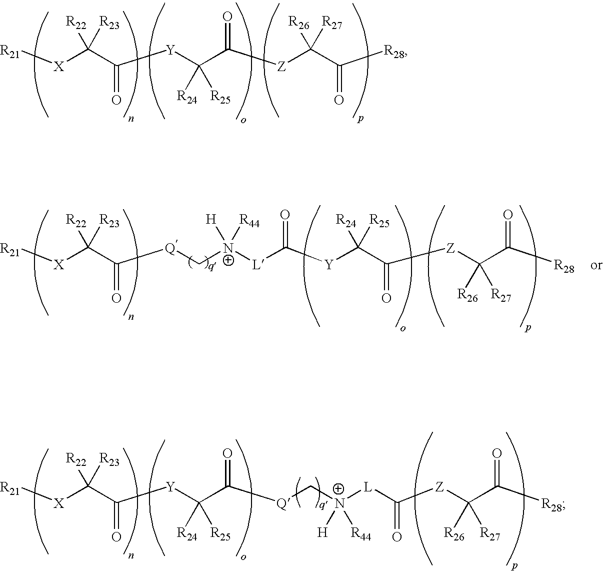

R₅₁ and R₆₁ are independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

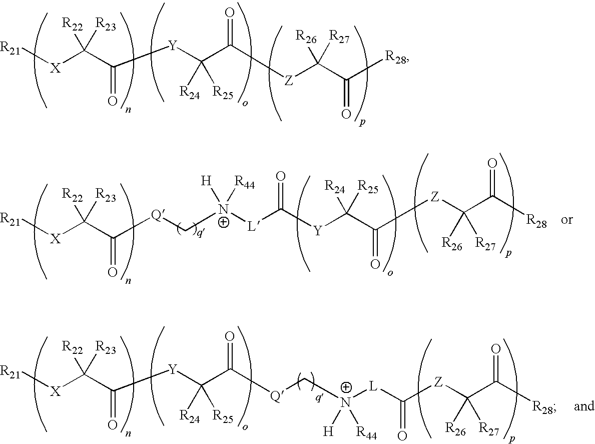

and R₂₀-R₂₉, R₄₃, R₄₄, n, o, p, q′, t, e, Q′, X, Y, Z and L′ are as previously defined;
provided that one and only one of R₅₀-R₆₅ is

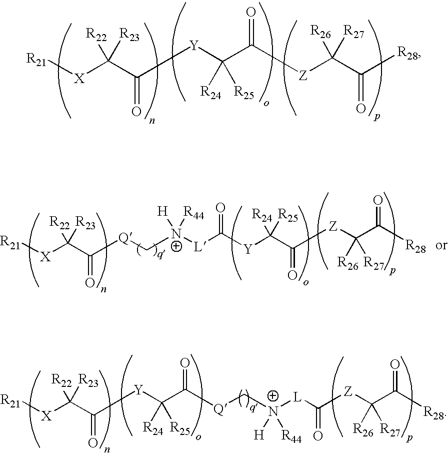

In some embodiments, R₆₁ is

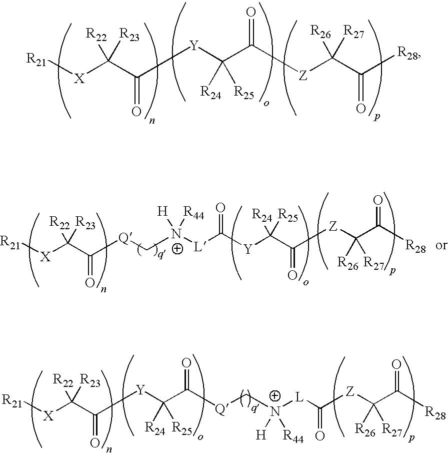

in the optical labeling molecules of structures (IV)-(XIV).

In some embodiments, an optical labeling molecule of structural Formula (XV) and structural formula (XV′), or a salt or solvate thereof, is provided:

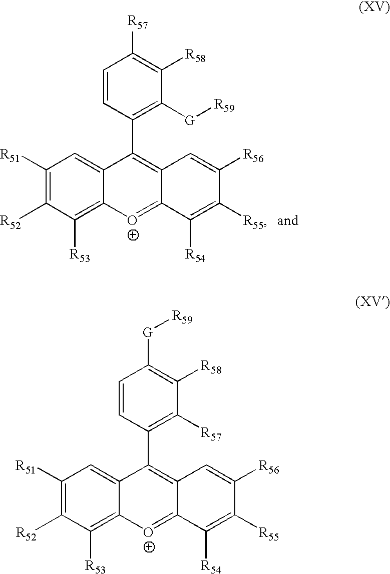

wherein the following definitions apply for formulae XV and XV′:
R₅₇-R₅₉ are each independently the same or different hydrogen, acyl, substituted acyl, alkoxy, substituted alkoxy, alkoxycarbonyl, substituted alkoxycarbonyl, alkyl, substituted alkyl, amino, substituted amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —(CH₂)ₙN⁺(CH₃)₃, —S(O)ₜR₂₀, —SO₃H, —(CH₂)ₙS(O)ₙOH, —(CH₂)ₙS(O)₂O⁻, —OP(O)(O⁻)₂, —(CH₂)ₙOP(O)(O⁻)₂

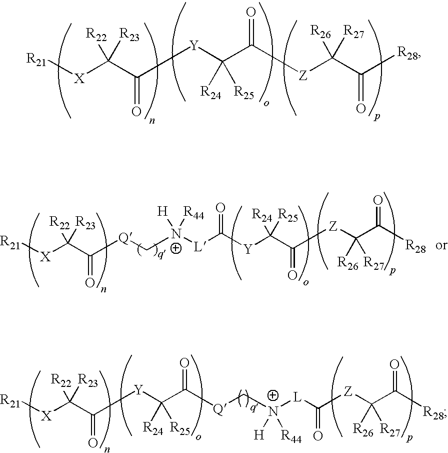

G is (CH₂)ₙ—(C(O))ₚ—N(R^{c})N(CH₂)qR^{c}, —(CH₂)ₙ—(C(O))—,

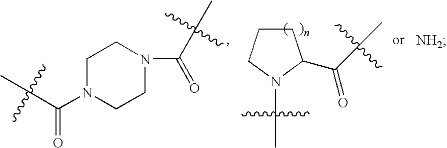

R^{c} is H, alkyl or can be taken together with the nitrogen atoms to which they are bonded form a cycloheteroalkyl or substituted cycloheteroalkyl ring;
R₂₀-R₂₉, R₄₃, R₄₄, n, o, p, q′, t, e, Q′, X, Y, Z and L′ are the same as defined above;
   provided that one and only one of R₅₇-R₅₉ is

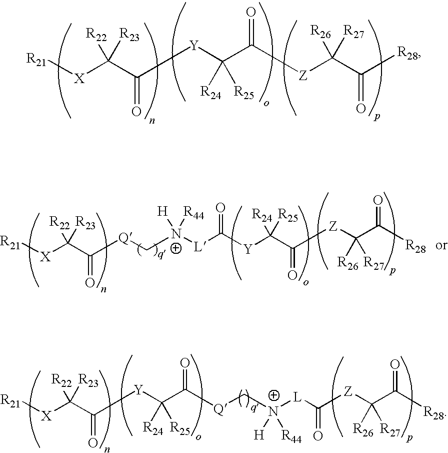

In one embodiment of Formula (XV) or (XV′), R₅₇ or R₅₉ is acyl, alkyl, substituted alkyl, alkoxy, heteroalkyl, substituted heteroalkyl, halo, nitro, —S(O)ₜR₂₀, —SO₃H, or

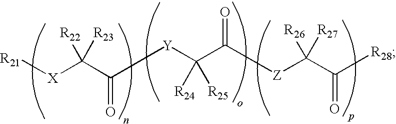

and R₅₁ to R₅₆ are each independently acyl, substituted acyl, alkyl, substituted alkyl, amino, substituted amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃, or

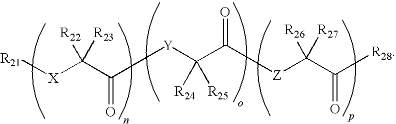

In one embodiment of Formula (XV) or (XV′), R₅₇ or R₅₉ is acyl, alkyl, substituted alkyl, alkoxy, heteroalkyl, substituted heteroalkyl, halo, nitro, —S(O)ₜR₂₀, or —SO₃H; and R₅₂ and R₅₅ are each independently acyl, substituted acyl, alkyl, substituted alkyl, amino, substituted amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃, or

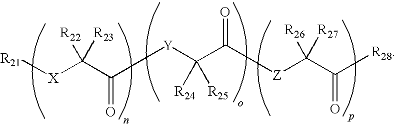

In one embodiment of Formula (XV) or (XV′), R₅₇ or R₅₈ are acyl, alkyl or substituted alkyl;

### R₅₉ is

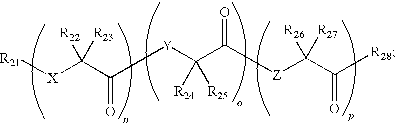

and R₅₂ and R₅₅ are each independently amino or substituted amino.
In one embodiment of Formula (XV) or (XV′), G is

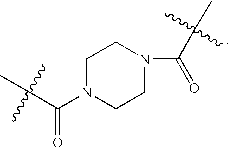

In one embodiment of Formula (XV) or (XV′), G is

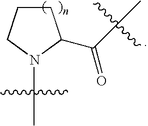

In some embodiments, the optical labeling molecule of structural Formula (XV or XV′), or a salt or solvate thereof, include the compound of Table 4.

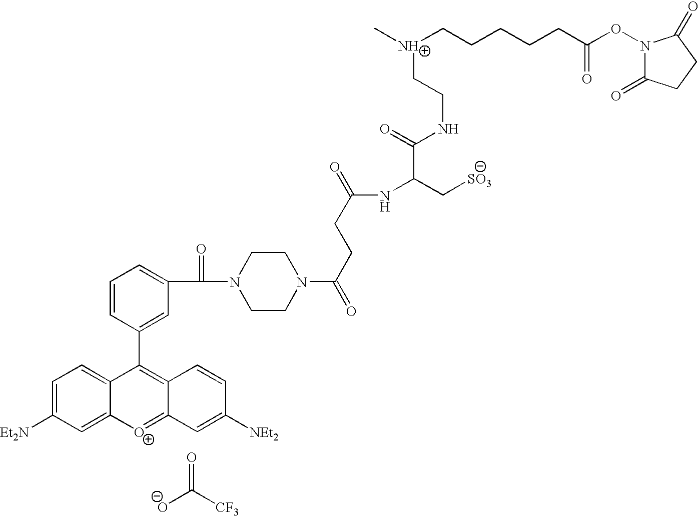

185

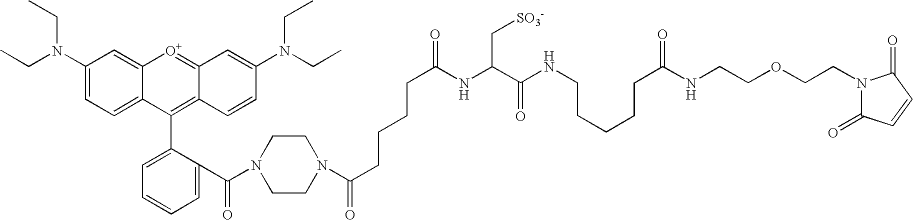

202

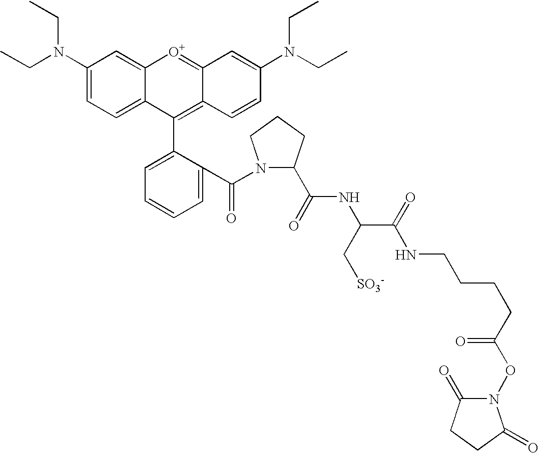

192

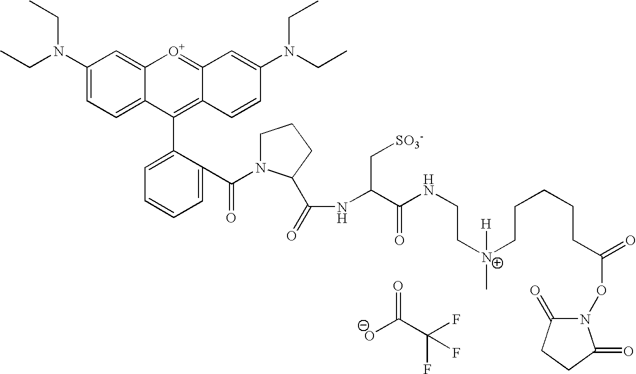

344

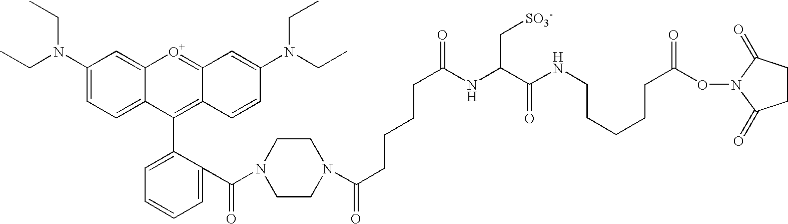

189

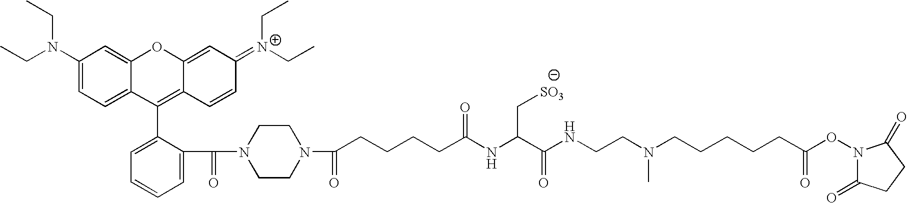

345

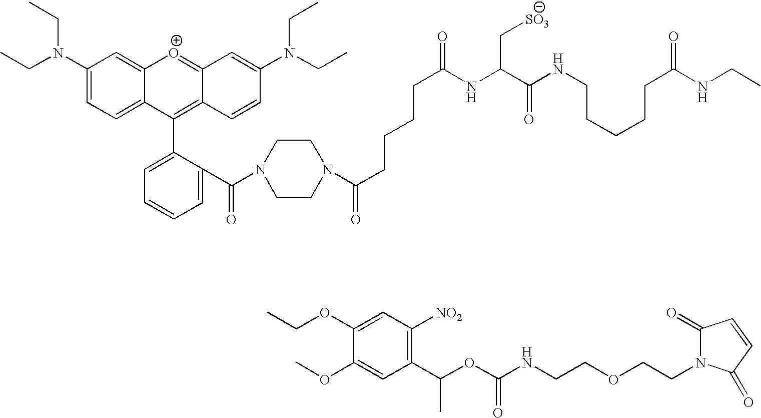

402

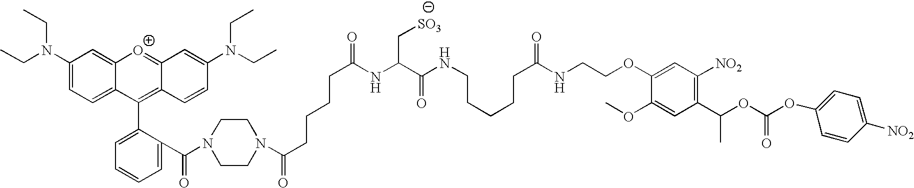

403

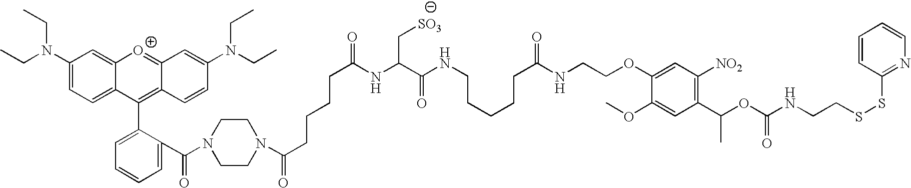

404

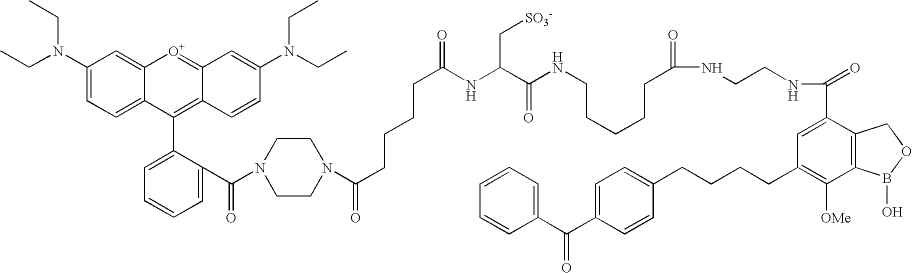

405

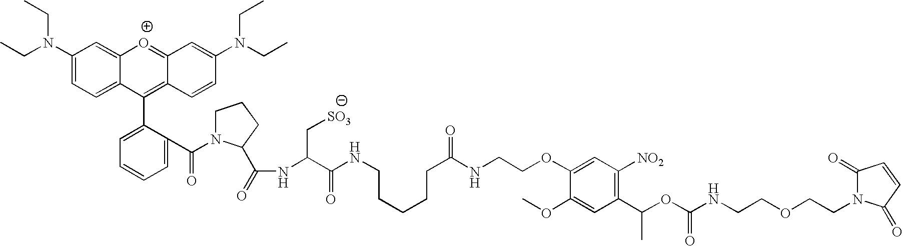

406

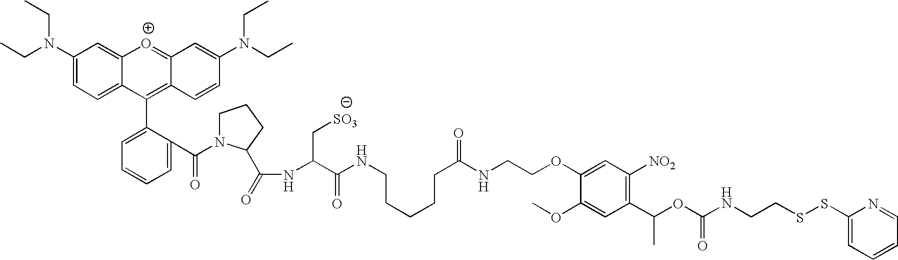

407

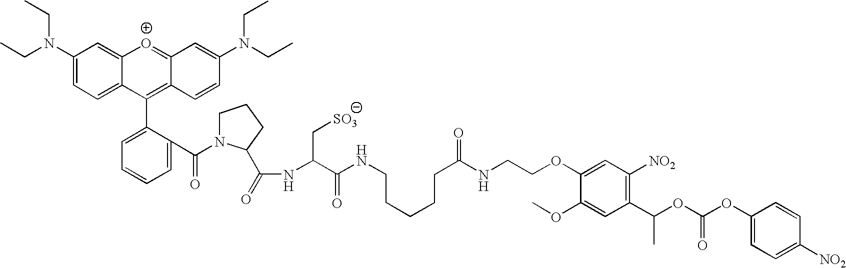

408

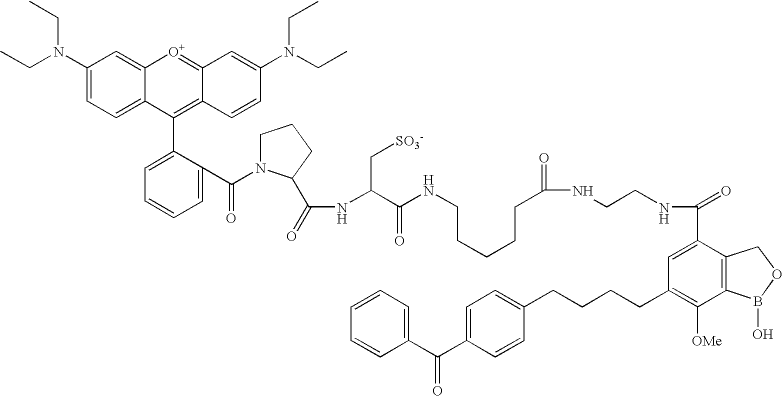

409

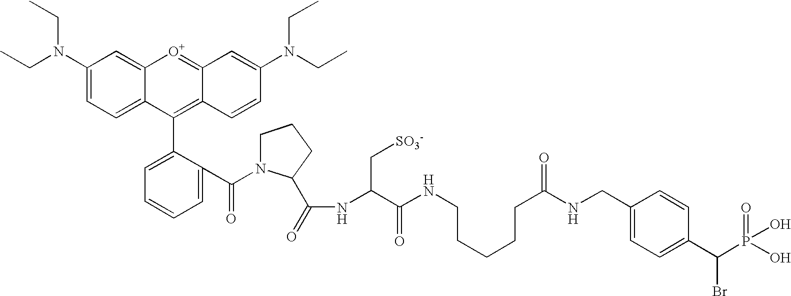

410

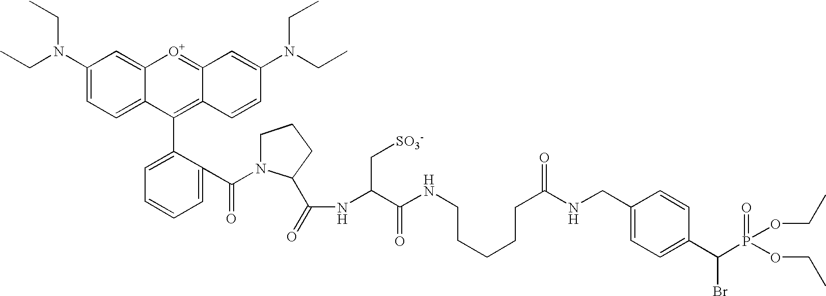

411

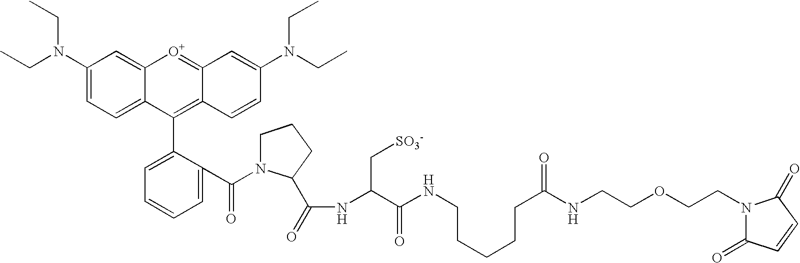

412

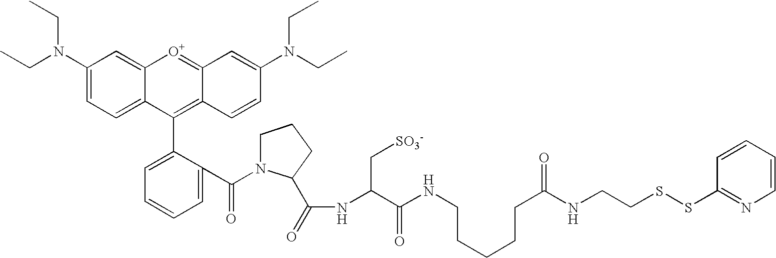

413

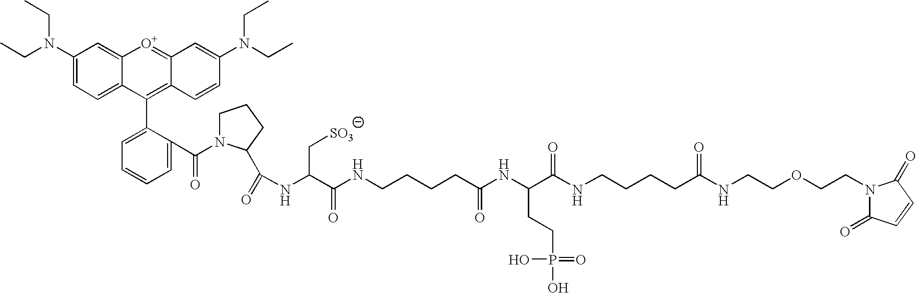

414

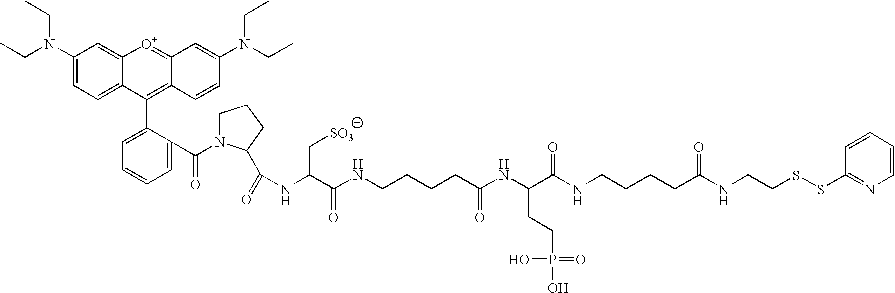

415

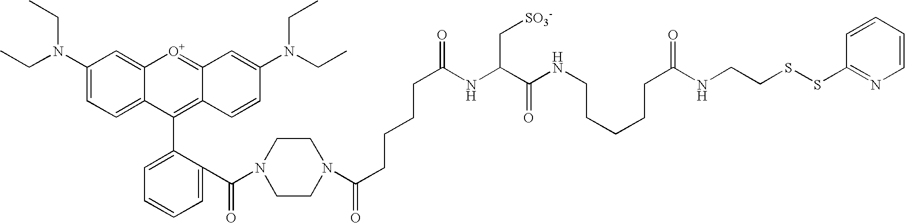

203

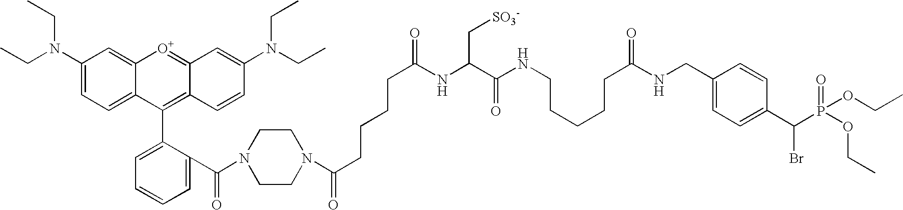

204

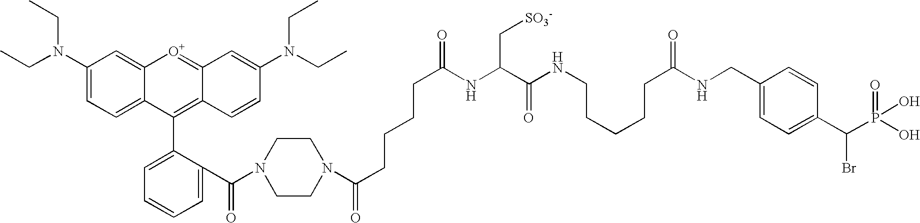

401

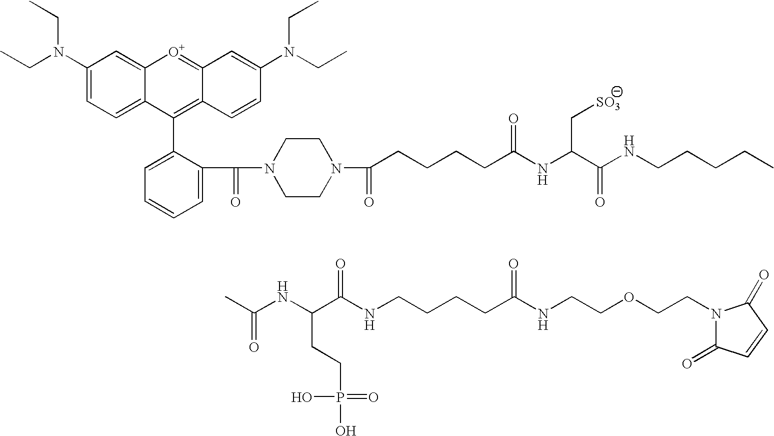

194

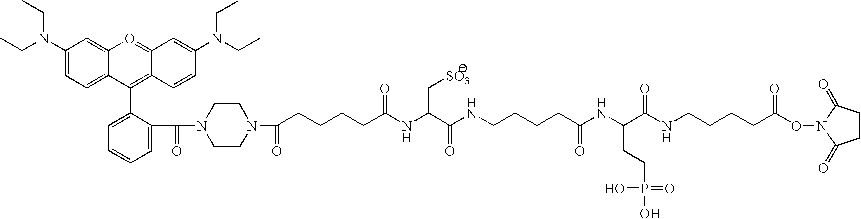

193

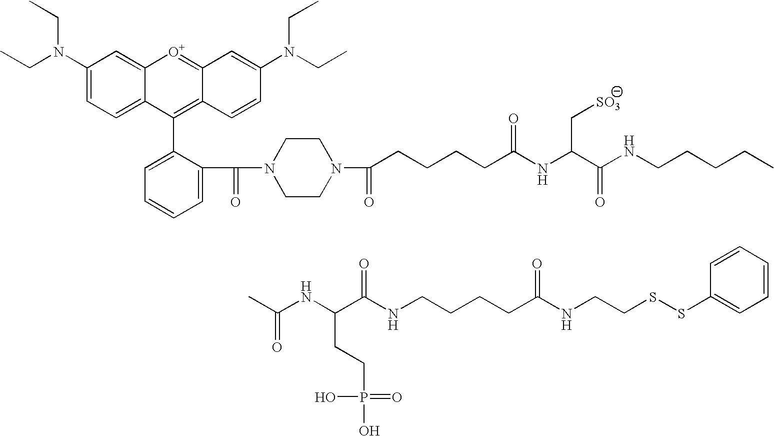

346

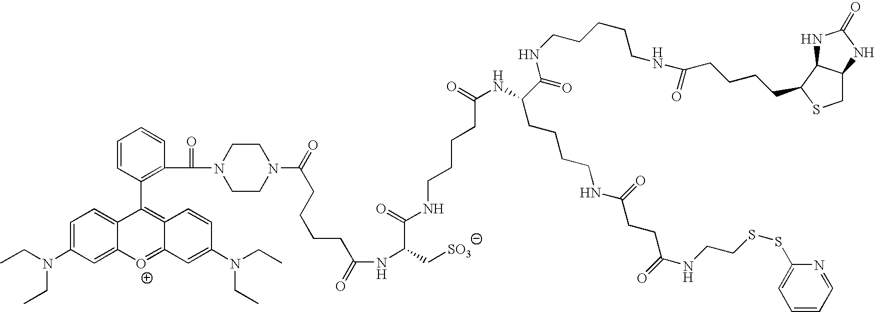

205

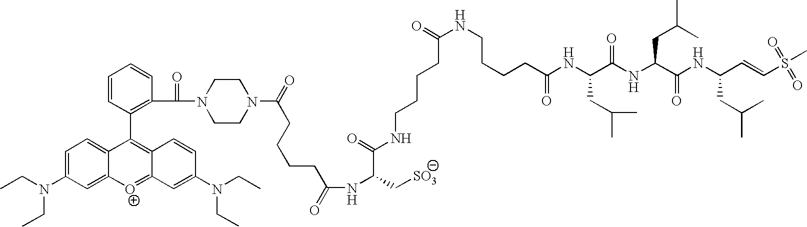

213

In some embodiments, a optical labeling molecule of structural Formula (XVI), or a salt or solvate thereof, is provided:

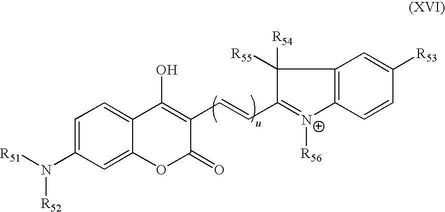

wherein:
u is 0, 1, 2, 3, 4, or 5;
R₅₃-R₅₅ are independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —S(O)ₜR₂₀, —SO₃H,

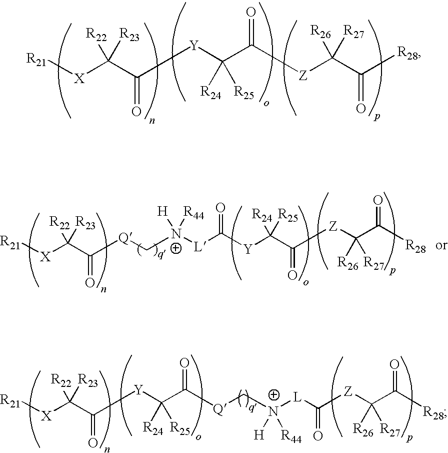

R₅₁, R₅₂ and R₅₆ are independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

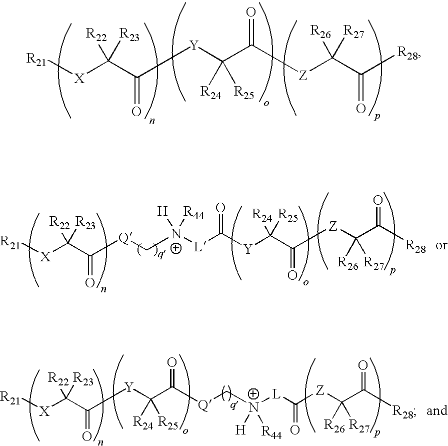

and R₂₀-R₂₉, R₄₃, R₄₄, n, o, p, q′, t, e, Q′, X, Y, Z and L′ are as previously defined;
provided that one and only one of R₅₀-R₅₉ is

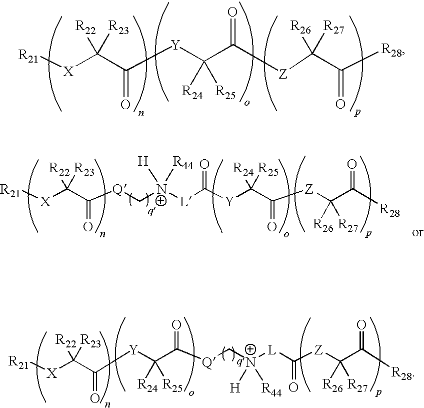

In some embodiments, R₅₆ is

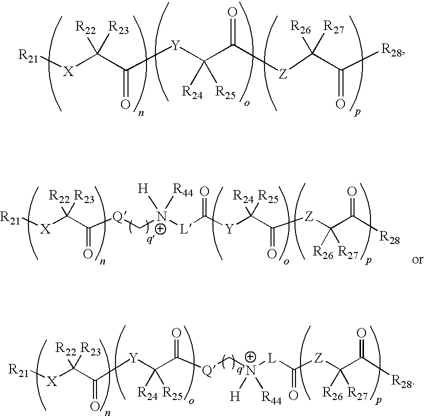

In some embodiments, a optical labeling molecule of structural Formula (XVII), or a salt or solvate thereof, is provided:

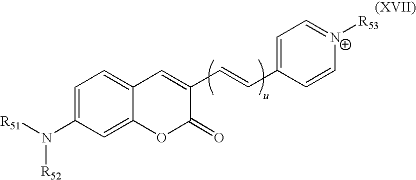

wherein:
u is 0, 1, 2, 3, 4, or 5;
R₅₁-R₅₃ are independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

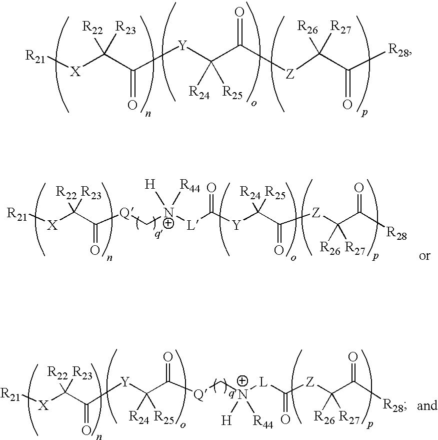

and R₂₀-R₂₉, R₄₃, R₄₄, n, o, p, q′, t, e, Q′, X, Y, Z and L′ are as previously defined;
provided that one and only one of R₅₀-R₅₃ is

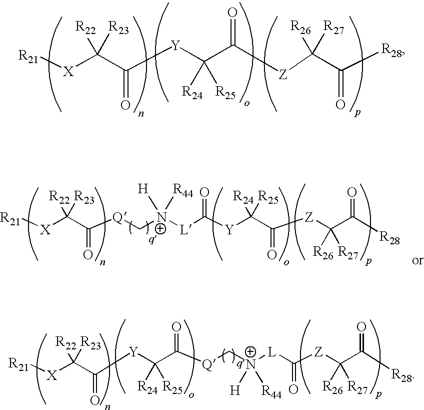

In some embodiments, a optical labeling molecule of structural Formula (XVIII), or a salt or solvate thereof, is provided:

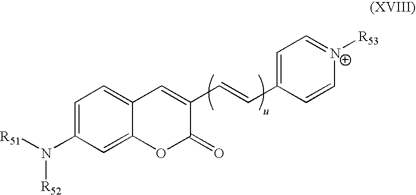

wherein:
u is 0, 1, 2, 3, 4, or 5;
R₅₁-R₅₃ are independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃,

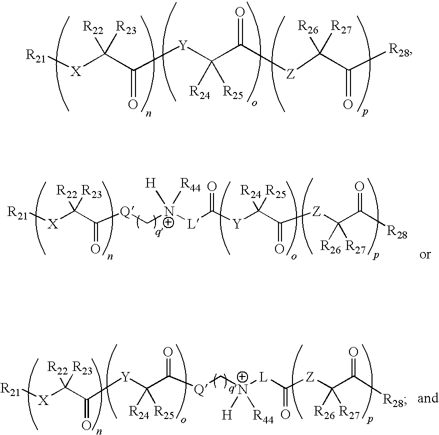

R₂₀-R₂₉, R₄₃, n, o, p, q′, t, e, Q′, X, Y, Z and L′ are as previously defined.
provided that one and only one of R₅₀-R₅₃ is

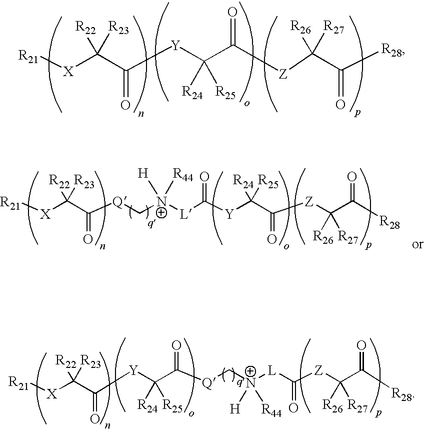

In some embodiments, R₃ is

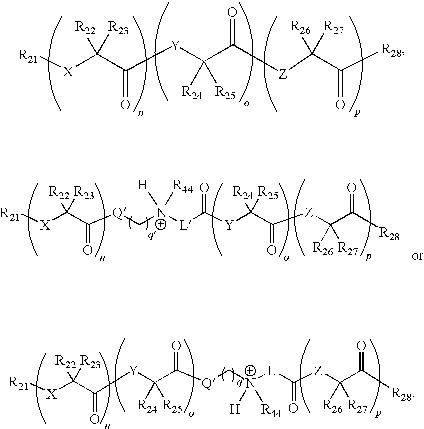

In some embodiments, an optical labeling molecule of structural Formula (XIX), or a salt or solvate thereof, is provided:

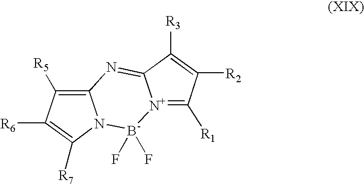

wherein: R₁-R₃ and R₅-R₇ are as previously defined in claim 1 provided that provided that one and only one of R₁-R₃ and R₅-R₇ are

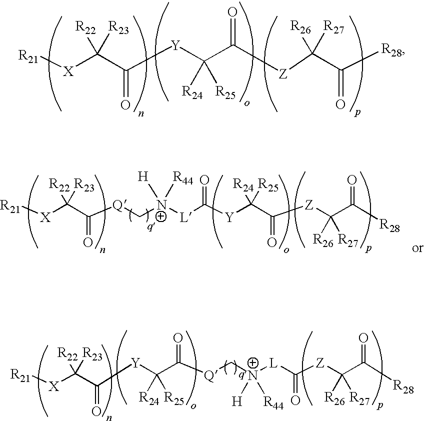

In some embodiments, an optical labeling molecule of structural Formula (XX), or a salt or solvate thereof, is provided:

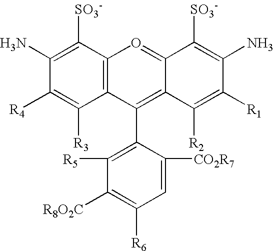

wherein: R₁-R₇ are as previously defined in claim 1 and R₈ is defined identically to R₁-R₇ provided that provided that one and only one of R₁-R₈

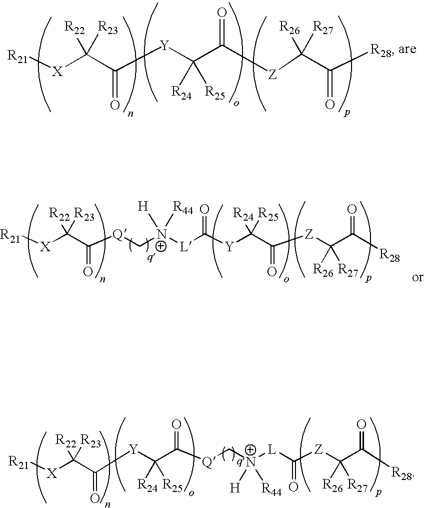

In some embodiments, an optical labeling molecule of structural Formula (XXI), or a salt or solvate thereof, is provided:

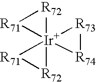

### Wherein:

R₇₁-R₇₄ are independently the same or different aryl, substituted aryl, heteroaryl, or substituted heteroaryl and R₇₂-R₇₄ are preferentially heteroaryl or substituted heteroaryl, and R₇₁-R₇₂ may be connected or substituted with one or more aryl, substituted aryl, heteroaryl or substituted heteroaryl rings R₇₃-R₇₄ may be connected or substituted with one or more aryl, substituted aryl, heteroaryl or substituted heteroaryl rings and contain at least one amino, substituted amino, acyl, substituted acyl, or

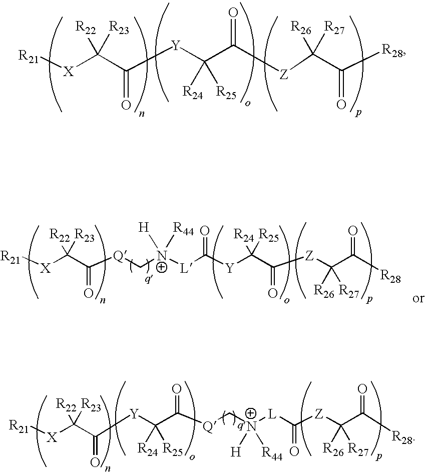

In another embodiment, an optical labeling molecule of structural Formula (XXI), or a salt or solvate thereof, is chosen from Table 5:

TABLE 5

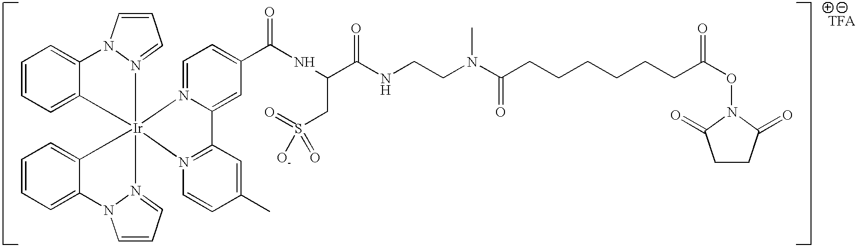

347

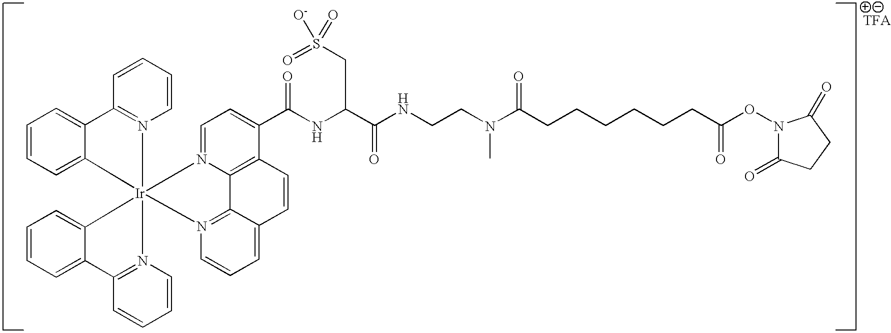

348

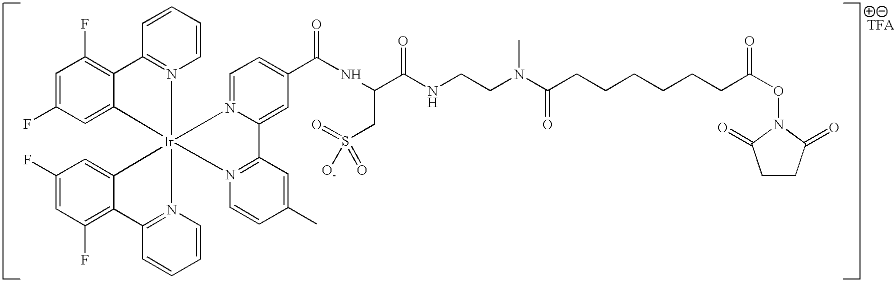

349

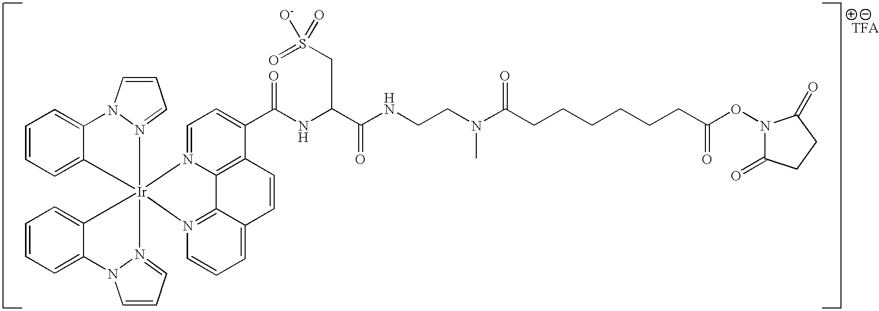

350

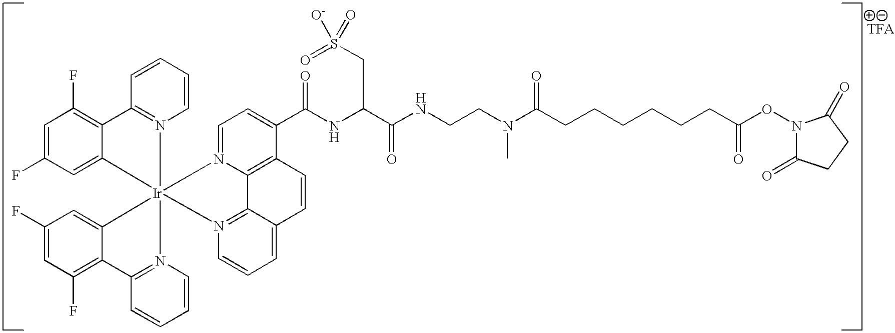

351

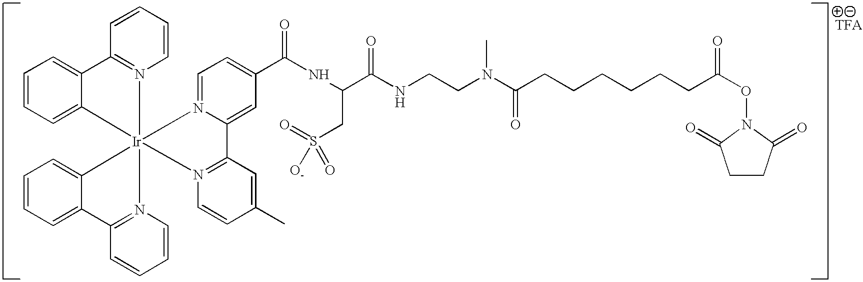

276

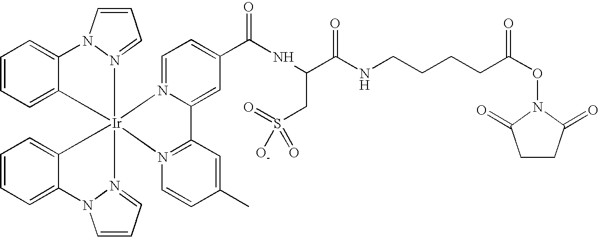

352

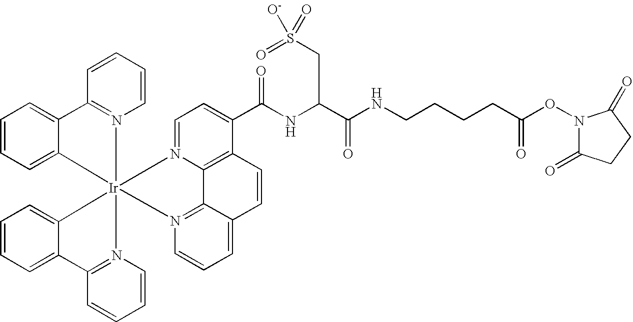

353

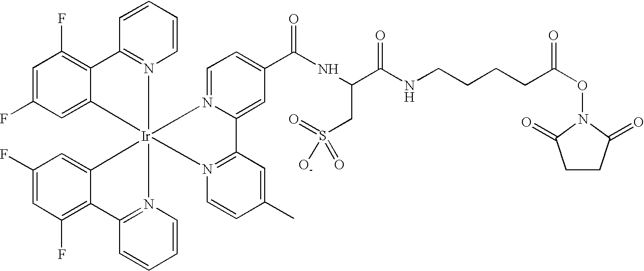

354

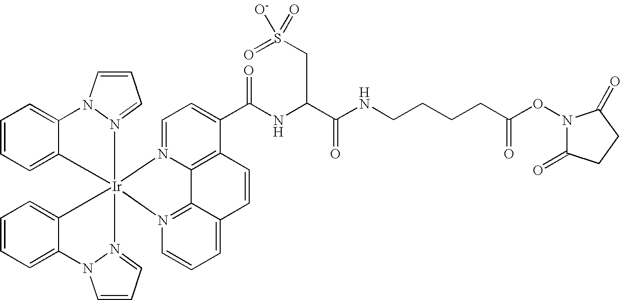

355

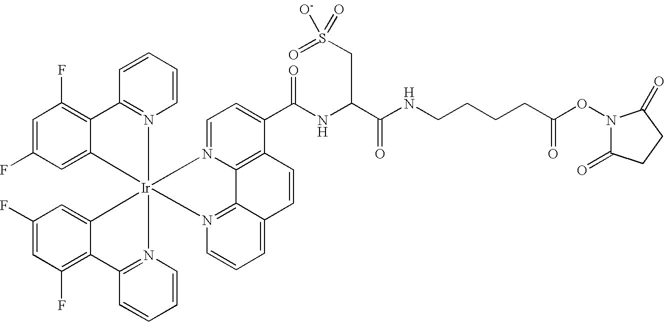

356

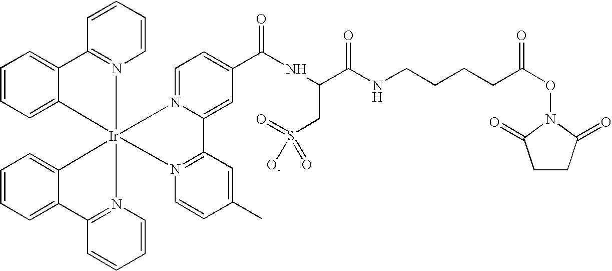

357

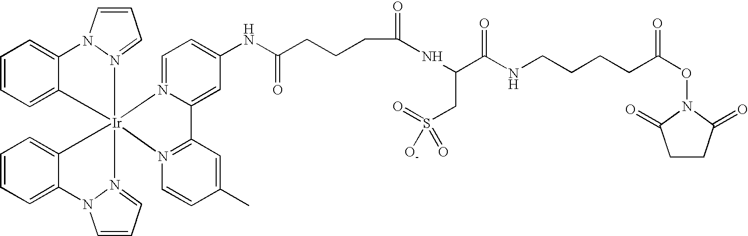

358

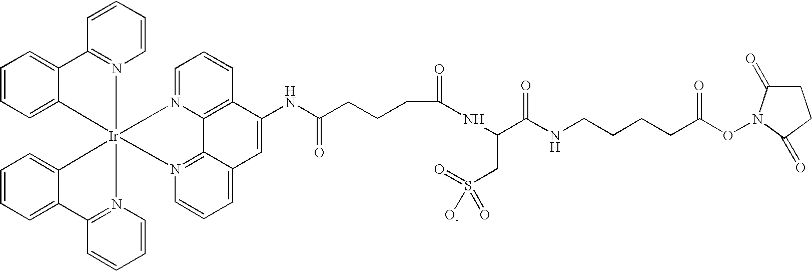

359

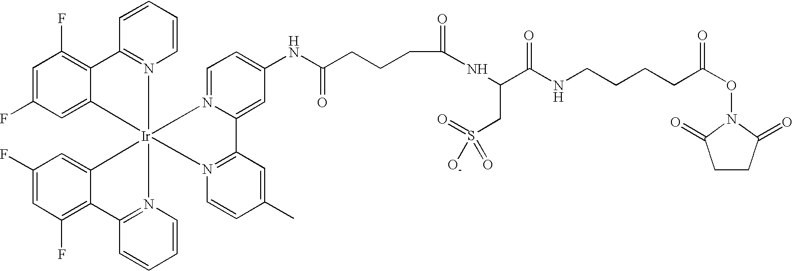

360

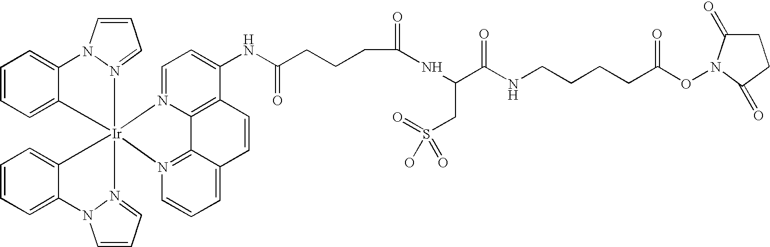

361

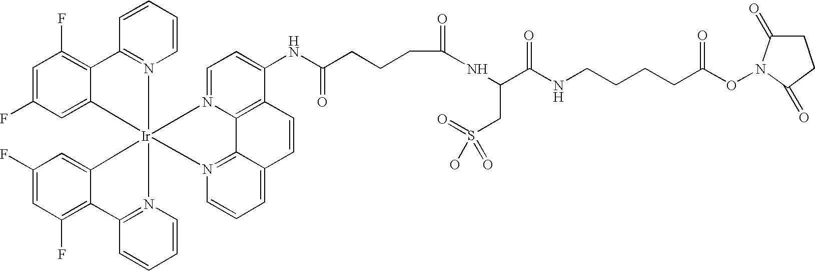

362

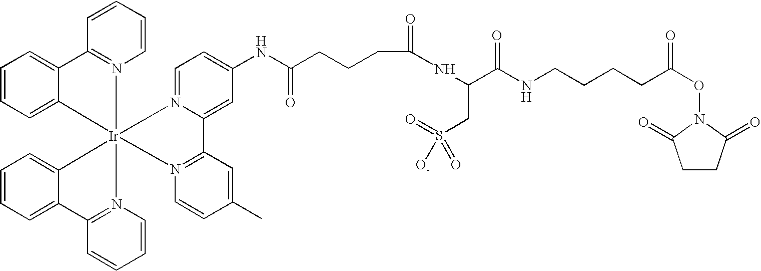

363

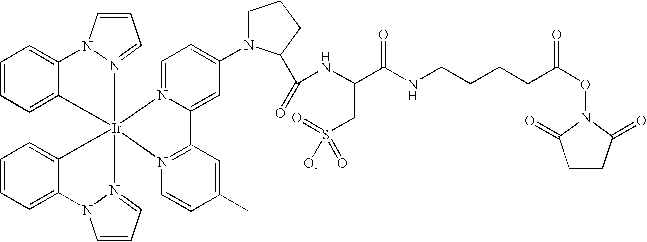

364

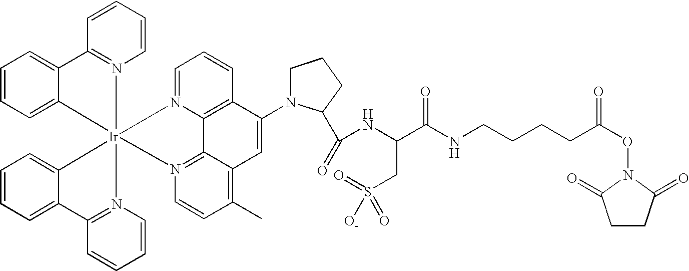

365

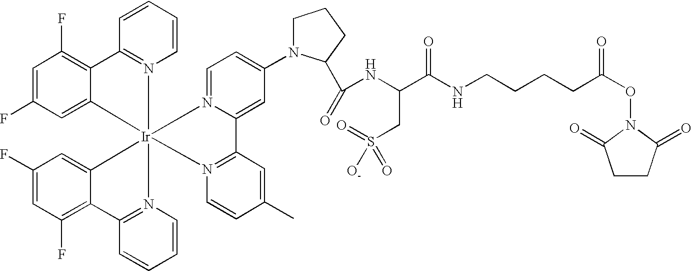

366

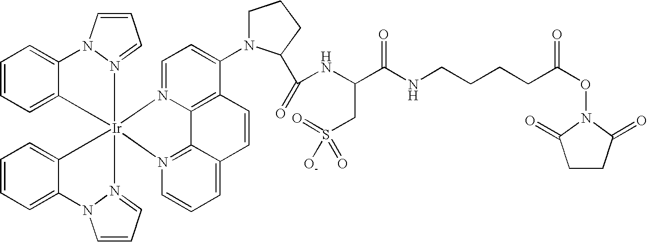

367

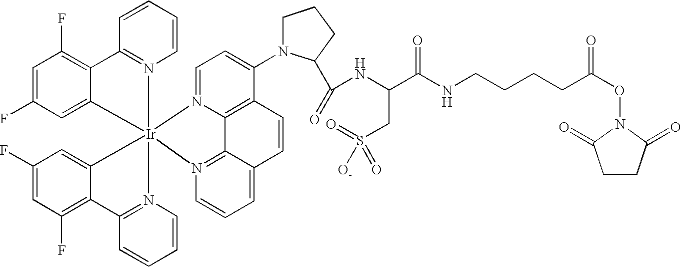

368

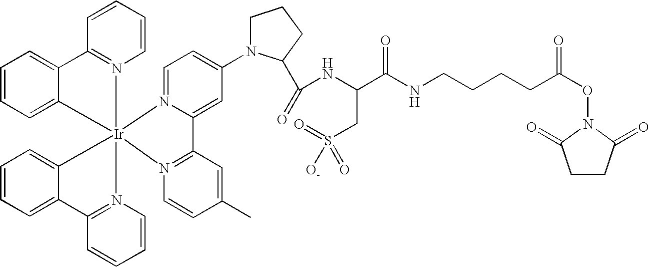

369

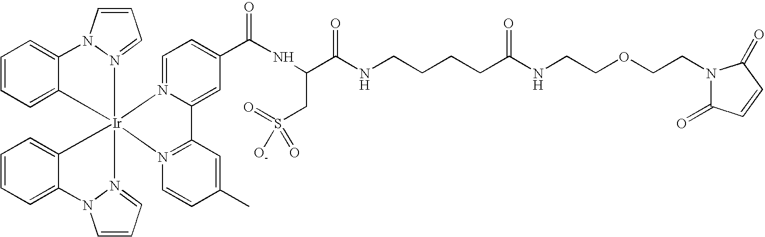

416

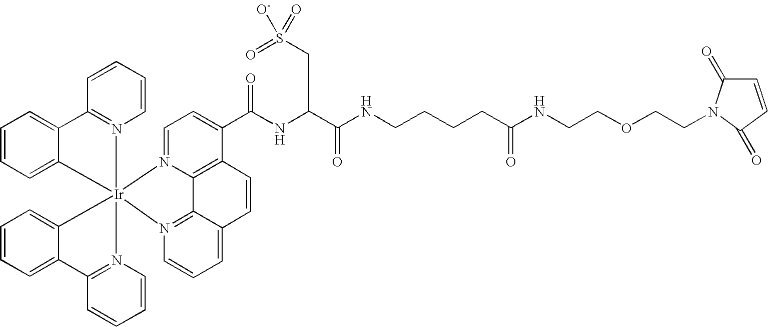

417

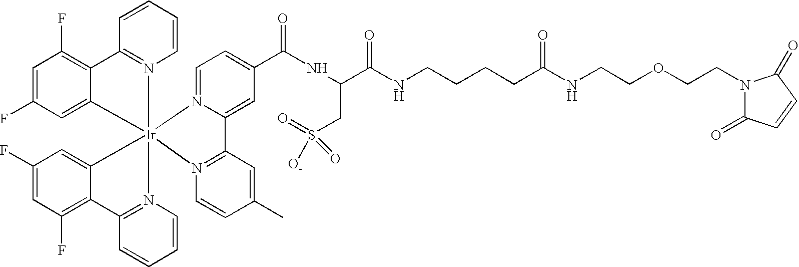

418

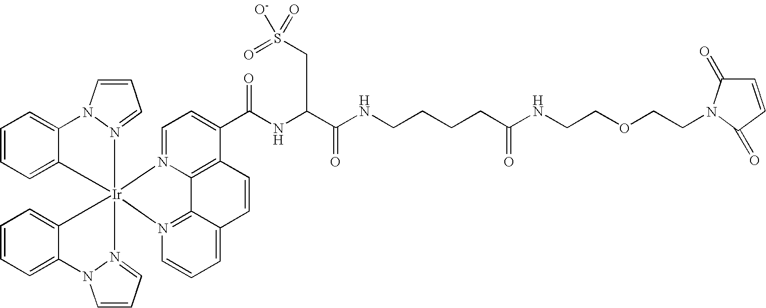

419

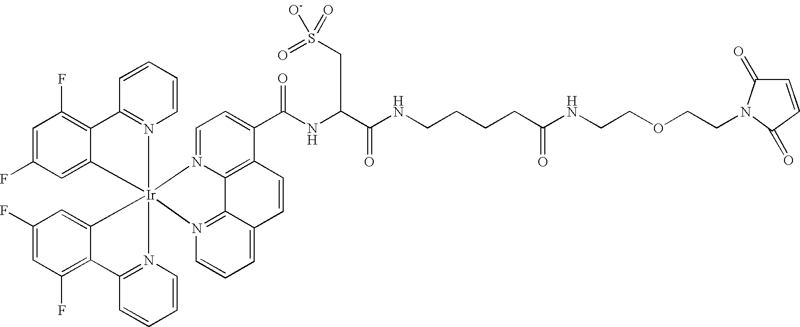

420

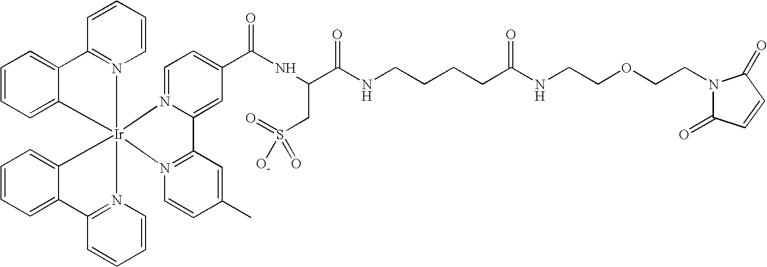

421

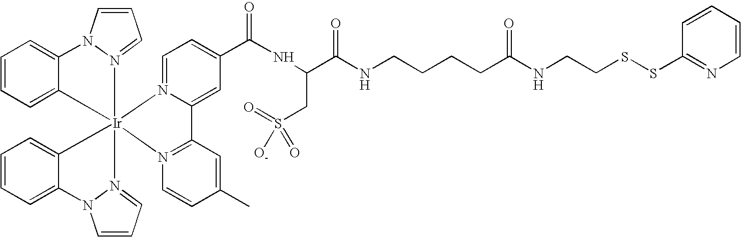

422

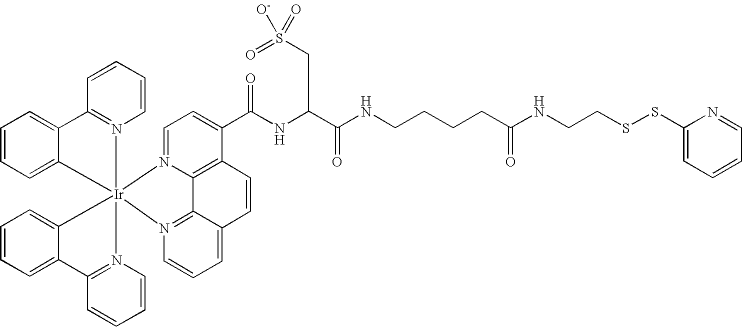

423

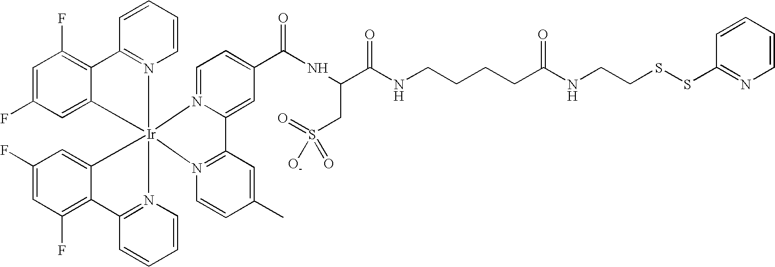

424

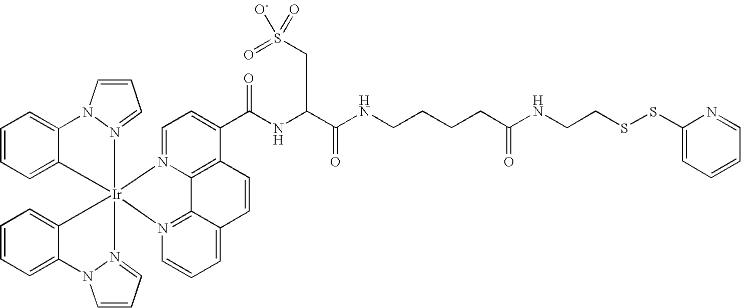

425

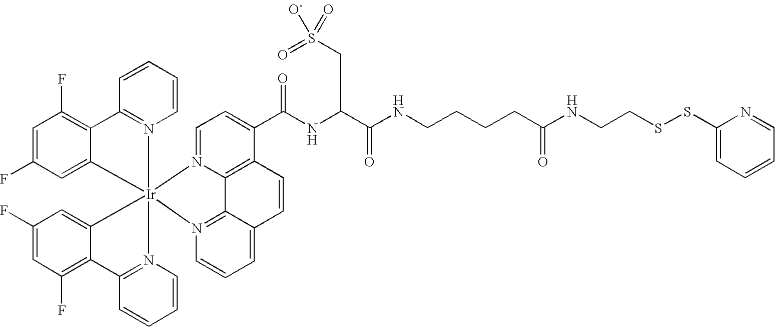

426

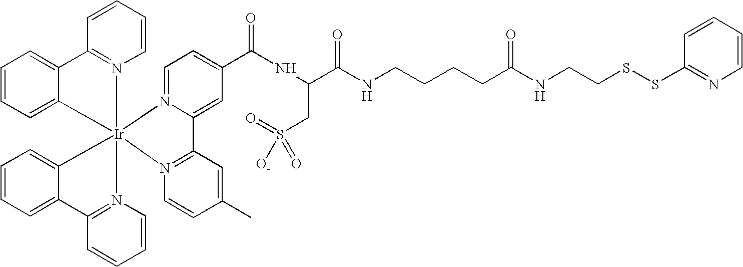

427

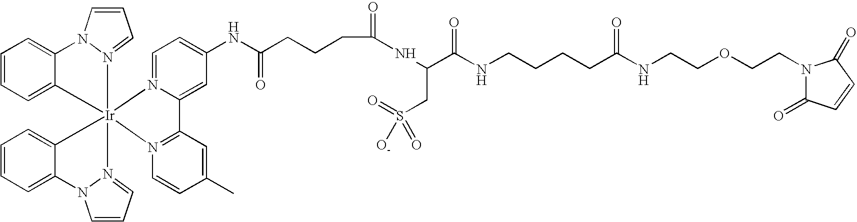

428

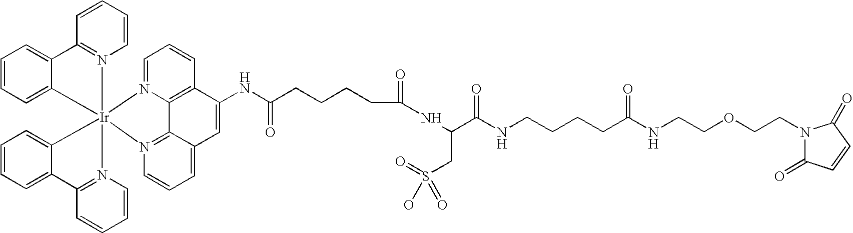

429

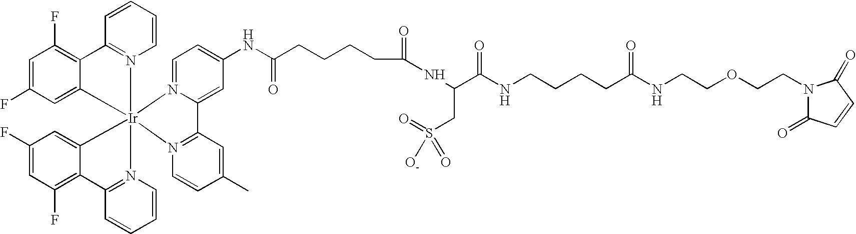

430

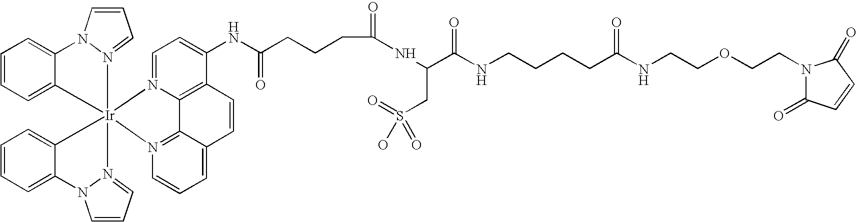

431

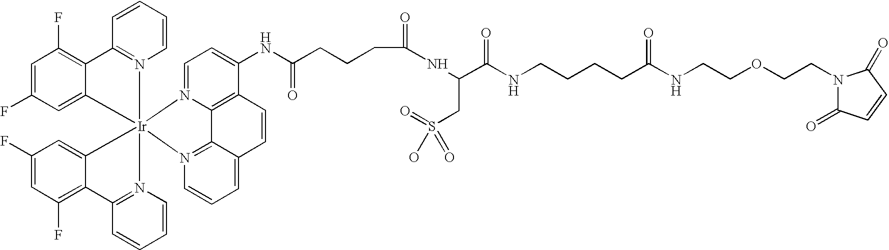

432

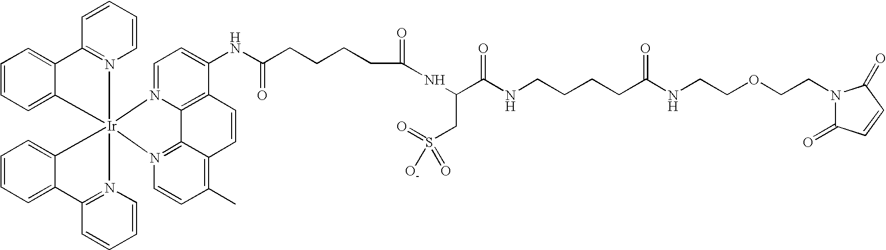

433

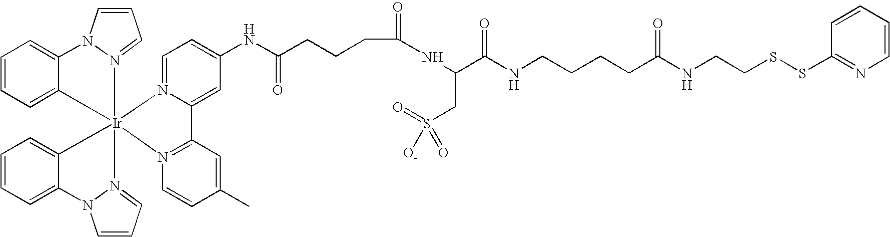

434

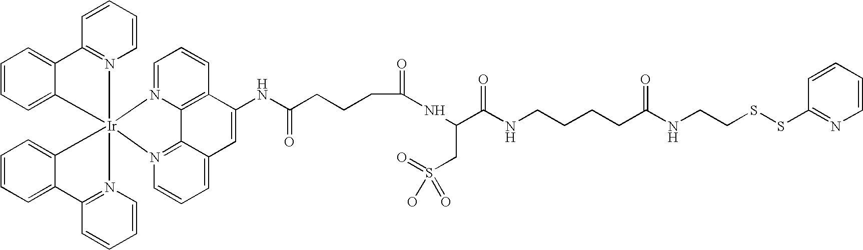

435

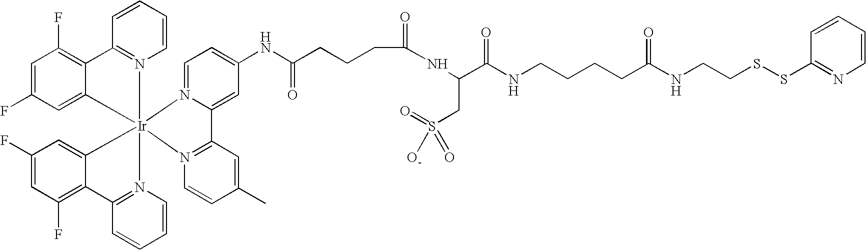

436

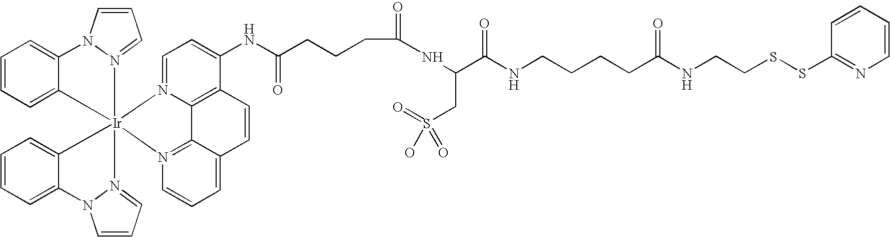

437

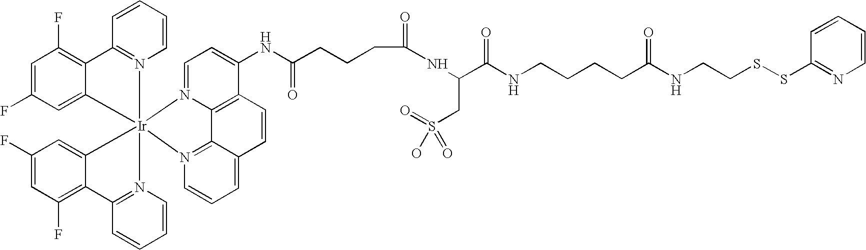

438

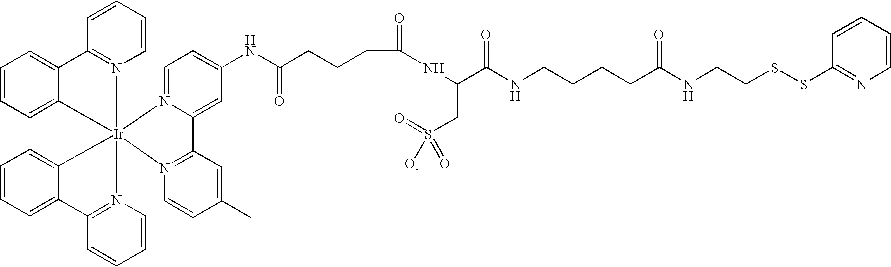

439

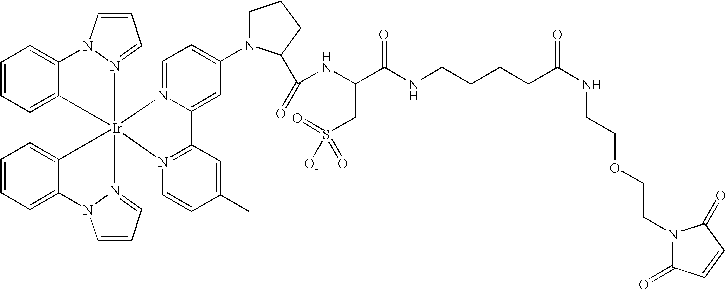

440

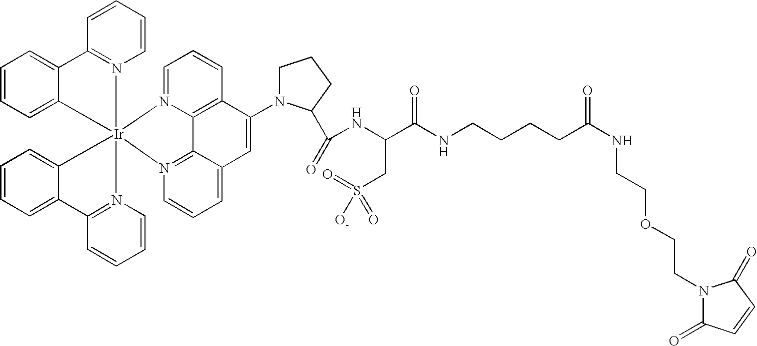

441

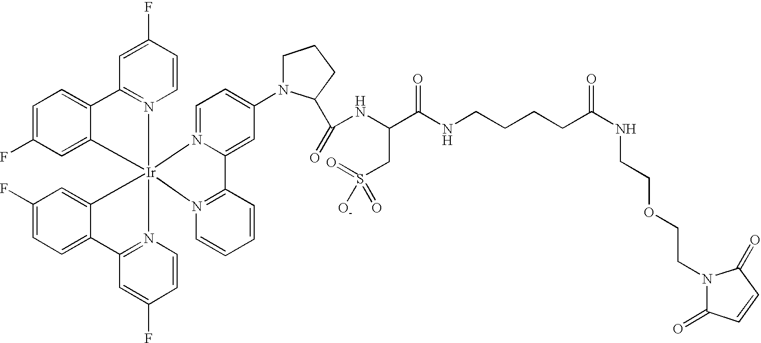

442

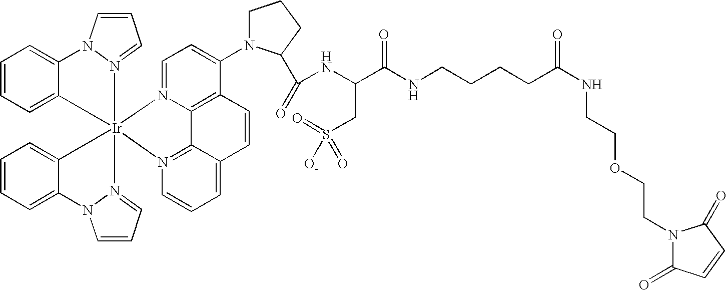

443

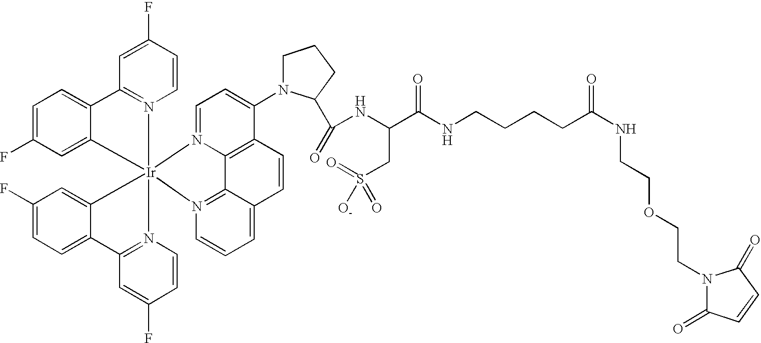

444

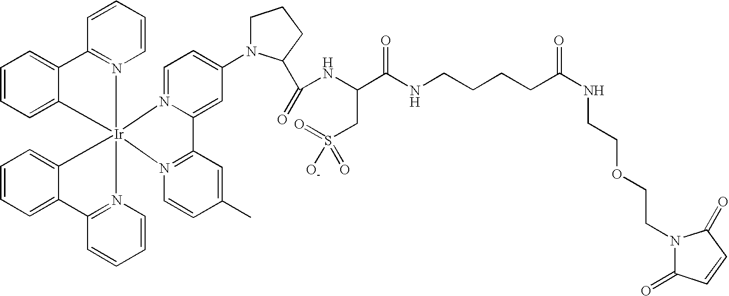

445

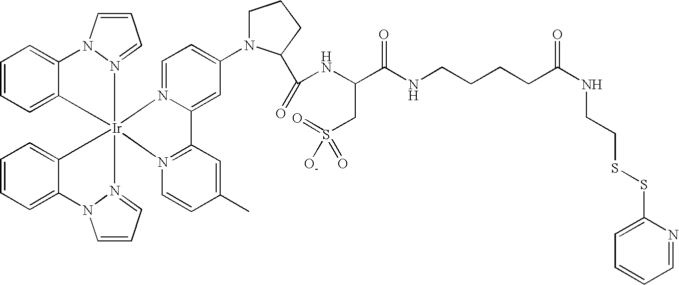

446

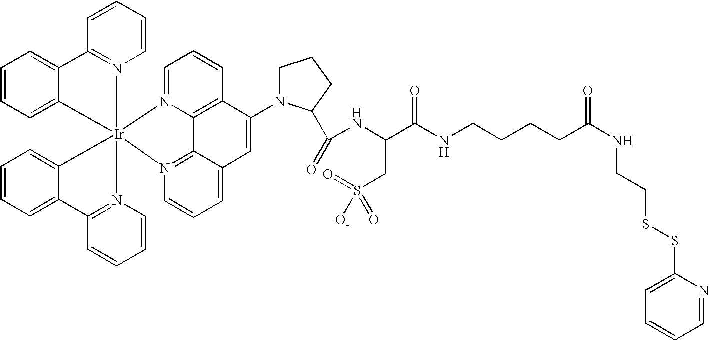

447

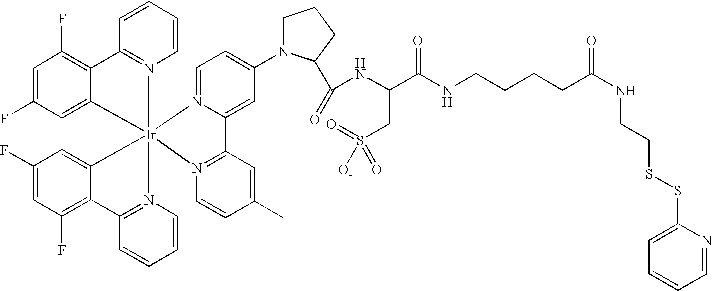

448

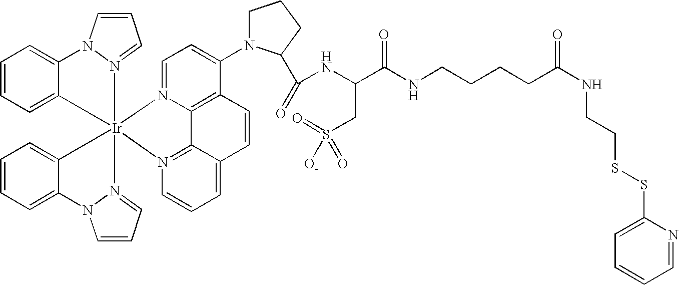

449

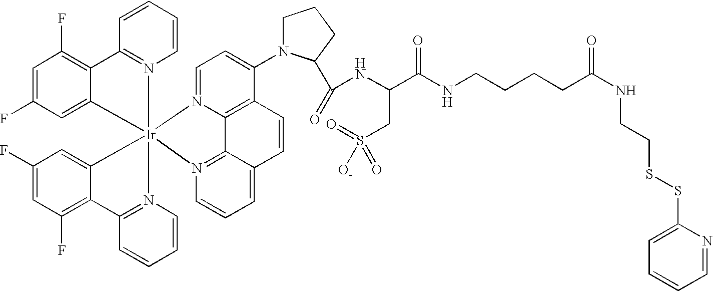

450

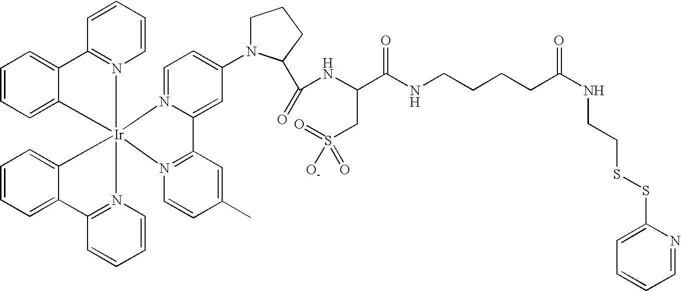

451

In some embodiments, the optical labeling molecules of Formulas (I)-(XXI) include at least one zwitterion pair. In other embodiments, the optical labeling molecules of Formulas (I)-(XXI) have between one and four zwitterion pairs. In still other embodiments, the optical labeling molecules of Formulas (I)-(XXI) have between one and three zwitterion pairs. In still other embodiments, the optical labeling molecules of Formulas (I)-(XXI) have a net positive charge. In still other embodiments, the optical labeling molecule is an Alexa 488 dye and has at least one added zwitterionic pair.

The optical labeling molecules described herein may be used in a wide variety of applications. In some embodiments, a method of labeling a protein using any of the above-described optical labeling molecules is provided where the optical labeling molecule is contacted with a target protein to form a labeled protein. The efficiency of forming the labeled protein is affected, for example, by pH, buffer, salts, temperature, other reagents, etc. as is known to those of skill in the art. In some embodiments, the protein is contacted with the optical labeling molecule between about pH 8.0 and about pH 8.5. Exemplary buffers include, but are not limited to, phosphate, phosphate/borate, tertiary amine buffers such as BICINE and borate. Other reagents which may be added to the labeling reaction mixture include various detergents, urea and thiourea.

The number of optical labeling molecules per labeled protein and the relative fluorescence of the optical labeling molecules on differently labeled proteins can be determined using methods well known to those of skill in the art. For example, the number of optical labeling molecules per labeled protein and the relative fluorescence of optical labeling molecules on different labeled proteins can be determined by separating the labeled proteins from the free optical label, using HPLC gel filtration with in-line fluorescence and absorbance detection or by numerous different gel-filtration columns. The ratio of hydrolyzed and unreacted optical label can be determined by RP-HPLC (reverse-phase HPLC), if desired, to help optimize labeling conditions. Isolated, labeled proteins can be incubated and re-run on gel filtration to determine the stability of the protein-optical label molecule complex. (Miyairi et al., (1998) Anal Biochem. 258(2):168-75; Mills et al., (1998) J Biol Chem. 273(17):10428-35; Kwon et al., (1993) Biochemistry, 32(9):2401-8).

In some embodiments, a plurality of target protein samples are labeled with different optical labeling molecules. By “different optical labeling molecule” is meant optical labeling molecules which exhibit different optical properties. For example, different optical labeling molecules include optical labeling molecules with fluorescent zwitterionic dye moieties, where each dye exhibits a different fluorescence spectra. In some embodiments, each optical labeling molecule has similar physical characteristics. By “similar physical characteristics” is meant that each optical labeling molecule has similar size, charge and isoelectric point characteristics to minimize any shifts in isoelectric point or chromatographic mobility between the labeled and unlabeled proteins. Optical labeling molecules that have similar physical characteristics are preferable to minimize relative changes in physical characteristics of the protein that arise as a result of the presence of the optical labeling molecule on the protein. For example, the presence of a labeling molecule on the protein may result in a change in gel mobility or electrophoresis mobility of the labeled protein relative to the unlabeled protein. When each labeling molecule of the family has similar physical characteristics, the plurality of labeled proteins labeled with different dyes will retain sufficiently similar physical characteristics to minimize differences in separation.

The most sensitive protein parameters in 2D gel analysis perturbed by labeling are the isoelectric point and solubility of the labeled molecule at or near the isoelectric point. 2D gels have modest resolution by mass and so labeling with different numbers of dyes generally does not change the apparent mass in a very significant manner on 2D gels, especially for larger proteins. The optical labeling molecules described herein increase the solubility of proteins, especially at the isoelectric point, but do not change the isoelectric point of the labeled proteins significantly when they contain titratable groups that replace the acid/base behavior of the functional group on the protein which reacts with the optical labeling molecule. The increased solubility of the labeled proteins is especially valuable for saturation thiol labeling. As a result, the plurality of proteins labeled with different dyes generally exhibit similar, but not necessarily identical mobility patterns, in gel electrophoresis and will also be similar, but not necessarily identical in gel mobility to the unlabeled proteins. The matching step of the described method of differential analysis of proteins described herein is performed to adjust the images to compensate for differences in gel mobility among proteins labeled with different optical labeling molecules using gel pattern matching software. The slight differences in gel mobility between proteins labeled with different colored dyes can be adjusted and superimposed by gel pattern matching software that is widely available in the field and known to persons skilled in the field.

The optical labeling molecules described herein may be used in differential multiplex detection reactions of proteins. Accordingly, provided herein are families of different optical labeling molecules which may be used to label a plurality of target proteins. Each member of an individual family exhibits different optical properties but has similar physical characteristics to other molecules of the same family.

The optical labeling molecules described herein typically do not shift the position of labeled proteins in the first (isoelectric point) dimension of 2D gels. However, the position of labeled proteins are differentially shifted in the second (molecular weight) dimension of the 2D gels. The compensation for any differential shift of labeled proteins due to the optical labeling molecules described herein allows for use of a wide range of fluorescent dyes, dye molecular weights and dye-protein coupling chemistries for detection of relative levels of proteins. In some embodiments, use of different optical labeling molecules described herein simultaneously detects the relative amounts of posttranslational modifications and/or relative levels of enzyme activities on 2D gels.

Gels are scanned with light excitation, (e.g., laser excitation) to derive or provide fluorescence images of the differently colored optical labeling molecules. In a further embodiment, there are at least two optical labeling molecules employed in the analysis. There may be more than two optical labeling molecules. The fluorescent images of optical labeling molecules can also be obtained by computational deconvolution of full fluorescent spectra obtained from each pixel by hyperspectral imaging. The fluorescent background is subtracted from each image, to the extent that it is known or can be estimated. The fluorescent background subtraction can be “exact” with hyperspectral imaging, but this can be done only very approximately, if at all, from conventional images.

The range of fluorescence signal intensities are often greater than can be captured with fluorescent gel imagers which are limited to a 16 bit intensity. In such cases, the fluorescent gel images are first scanned at a detector sensitivity which does not saturate the strongest signals and one or more images are scanned at higher detector sensitivity which brings the weaker signals into the dynamic range of the detector and saturates the stronger signals. The outline of the saturated signals from the higher sensitivity images are used as a mask to cut off the saturated signal values. The images taken with the lower detector sensitivity are scaled up using 32 bit arithmetic to match the signal values taken at the higher sensitivity and used to fill in the peaks of the signal mask to create a images with greatly increased signal dynamic range. These images may be used directly to compare with the other colored images to identify up and down regulated protein spots, using 32 bit arithmetic. Alternatively, the logarithm of the 32 bit signals can be taken to create 16 bit images which can be analyzed with conventional gel image analysis programs that use 16 bit computer files.

The different colored images of the same 2D gel are matched, in an essential step, to adjust/morph the different-colored images to accommodate small shifts between the proteins labeled with the different colored fluorescent dyes. The matching of the different colored images of the 2D gels show patterns of systematic differences, with larger vertical shifts for smaller proteins and smaller vertical shifts for larger proteins.

Once the different colored images are matched, the matched images can be identically cropped, if desired, to remove poorly resolved features at the bottom, sides or top of the images. After the images are cropped, the total amount of fluorescence signal in each image is summed and the intensities of each feature in each fluorescent image is normalized by the total intensity of that image. If expanded dynamic range images are created in a 32 bit format the intensity normalization must be carried out in the 32 bit format before logarithmic or other compression is carried in a 16 bit format for calculation of intensity ratios. The ratios of fluorescent intensities of the images are calculated for several replications of the experiment and the proteins in the spots that show significant intensity changes (the level of significance is chosen by the investigator), as a function of the biological variables, can be identified and analyzed by mass spectrometry. General protein stains can be used to identify the location on the 2D gels of the unlabeled proteins, if the dye labeling protocol does not saturate the labeling sites. The multicolor matched image is then matched to the general protein stain image to identify the regions of the gels to analyze by mass spectrometry. A variety of methods can be used to transfer proteins or protein digests from 2D gels into mass spectrometers including, but not limited to, in-gel digestion and peptide extraction, electroelution and direct analysis of dried gels by laser desorption.

When multiply fluorescently labeled protein samples are separated by liquid chromatography or electrochromatography, fluorescence is measured from two or more colors and regions of the chromatographic eluants may be selected for mass spectral analysis. In some embodiments, the optical labeling molecules can be cleaved from labeled proteins (in some cases to regenerate the original functional groups) after the determining step, once the protein spots of interest are identified. The dye removal can enhance protease digestion for mass spectral analysis and can simplify protein and peptide identification and characterization by mass spectrometry.

In other embodiments, different isotopic tags are associated with differently-colored optical labeling molecules. In these embodiments, cleavage of the optical labeling molecule from the labeled protein can provide a protein still labeled with an isotopic label. Examples of such optical labeling molecules are those which are isotopically labeled between the cleavable group and the activating group. Accordingly, the ratios of proteins stained with different colored optical labeling molecules can be accurately determined by mass spectrometric analysis. These embodiments are significant when two or more protein species are in some of the gel spots or liquid chromatographic fractions which are analyzed by mass spectrometry. These embodiments result in the determination of the relative amounts of the separated labeled proteins in the different samples by the determination from the relative abundances of the isotopic tags by mass spectral techniques.

In some embodiments, proteins present in a sample of the extract of cells prior to exposure to physiological stimuli are labeled with an optical labeling molecule. Proteins present in the cell extract sample after exposure to the physiological stimuli are labeled with a different optical labeling molecule of the family. Additional samples may be labeled with still different optical labeling molecules, after different ranges of physiological stimuli are applied. The labeled proteins from cellular extracts are mixed and then simultaneously partially or fully separated into constituent components. The separated components are analyzed by observing the optical signals of the separated proteins, which identify protein components which are altered in expression level or posttranslational modification state, in response to the stimuli of interest. The altered protein components can then be further characterized by mass spectrometry using standard analysis techniques.

In some embodiments, the presence or absence of labeled proteins is analyzed to determine if a specific protein is affected by the presence or absence of a physiological stimuli. In other embodiments, the relative quantity (or ratios of expression) of the specific labeled proteins as a function of the stimulus is determined. In still other embodiments, the plurality of differently labeled protein samples are separated prior to determining the ratios of expression, enzyme activity, or posttranslational modification of the different labeled proteins. The differentially labeled proteins in the different samples may be separated using, for example, 1D gel electrophoresis, 2D gel electrophoresis, capillary electrophoresis, 1D chromatography, 2D chromatography, 3D chromatography, and further analyzed by mass spectroscopy. In some embodiments, a large number of labeled proteins are separated by 2D gel electrophoresis and the relative amounts of the proteins in different spots are determined by the relative strength of laser induced fluorescence emission and simultaneous multiplex analysis of the strength of the signals from the different fluorescence dyes.

In some embodiments, an optical labeling molecule having at least one amide bond in the derivative tail provides strong fluorescent signals that do not diminish with increasing pH employed during the separation of basic proteins on 2-D gels.

In some embodiments, the relative quantity of each differently labeled proteins are determined. The relative quantity of the different labeled proteins can be assessed, for example, by measuring the relative intensity of the optical signal emitted by each of the differently labeled proteins.

In other embodiments, the absolute quantity of differently labeled proteins is determined. Absolute quantity of a labeled protein can be assessed, for example, by including a known amount of an optically labeled protein as an internal standard. Absolute quantity can also be determined by including a known amount of an isotopically-labeled protein or peptide as an internal standard.

In some embodiments, a cleavable group is present in the optical labeling molecule between the dye moiety, the linker and the activator. After separating the differently labeled proteins as discussed above, the optical labeling molecule is cleaved from the labeled protein. The protein can then be analyzed, for example, using mass spectral techniques (Tao et al., (2003) Current Opinion in Biotechnology, 14:110-188; Yates, (2000) Trends Genet. 16: 5-8). The removal of the optical labeling molecule may also enhance protease digestion and/or efficiency of ionization for mass spectral analysis.

In some embodiments, an isotope label is present on the optical labeling molecule between the cleavable group and the activator moiety. After separating the differently labeled proteins as discussed above, the optical labeling molecule is cleaved from the target protein leaving an isotope label on the target protein. The relative amounts of the target proteins in samples labeled with different isotope labels can then be analyzed, using mass spectral techniques. In other embodiments, different isotope tags are associated with differently-colored optical labeling molecules. In this embodiment, the ratios of proteins stained with different colored optical labeling molecules can be accurately determined by mass spectrometric analysis. These embodiments are significant when two or more protein species are in some of the gel spots or liquid chromatographic fractions which are analyzed by mass spectrometry.

In some embodiments, optical labeling molecules with a net positive charge are provided. Such optical labeling molecules may be used in differential fluorescent detection in liquid chromatography separations and to detect peptides via mass spectrometry, using electron transfer dissociation (ETD) or electron capture dissociation (ECD) mass spectrometry.

The optical labeling molecules disclosed herein may reveal changes, such as relative amounts of protein, absolute amounts of protein, analysis of posttranslational modifications, analysis of enzyme activities, analysis of protein levels, analysis of cell or organelle surface-exposed proteins, phosphorylation states, nitrosylation states, and glycosidation states of proteins. Further changes may be those caused by biological variables in a plurality of protein posttranslational modifications including, but not limited to, phosphorylation, glycosidation, thiol oxidation/reduction, nitrosothiol, nitrotyrosine, ADP ribosylation, disulfide formation, glycoslyation, carboxylation, acylation, methylation, sulfation, and prenylation, etc. Further, optical labeling molecules disclosed herein may reveal changes caused by biological variable in a plurality of enzyme activities including, but limited to, proteases, caspases, kinases, phosphatases, glycosidases.

In some embodiments, the phosphorylation state of proteins in the cells is determined. In these embodiments, unstimulated cells are labeled with ³³P phosphate and the protein extract of the cells is labeled with a first optical labeling molecule. Cells that have been exposed to a growth factor or other stimulus are labeled with ³²P phosphate and a second different optical labeling molecule. Preferably, the first and the second optical labeling molecules are chosen from the same set of optical labeling molecules so that the optical signal is different but the physical characteristics are similar. The labeled extracts of cells are mixed and simultaneously separated by a method described above. The labeled extracts are analyzed with optical scanning to determine protein expression ratios between the stimulated and unstimulated cells. The gel is sandwiched between two phosphoimaging detector plates with a thin metal foil in between the gel and the phosphoimager plate on one side of the gel. The phosphoimager plate on the side with no foil responds to ³²P+³³P whereas the phosphoimager plate on the side with the metal foil only detects ³²P since the beta radiation from ³³P is blocked by the thin metal foil. The phosphoimager plates are read and the ratios of the signals for the two plates are analyzed to determine the relative amount of phosphorylation on each protein on the gel. The method can be used to determine phosphorylation levels of each protein on a gel by using antibodies or other labels, e.g., antiphosphothreonine antibodies and a chemical labeling method for phosphoserine and phosphothreonine groups on gel-separated proteins. After the proteins are separated on the gel and expression ratios measured by laser scanning the gels, the proteins can either be further analyzed on the gel or transferred to blotting membranes for further analysis.

In some embodiments, the gel or blot is incubated in strong base (e.g., 1 M barium hydroxide) for a sufficient time to beta-eliminate the phosphate groups from phosphoserine and phosphothreonine. An optical labeling molecule containing a thiol group is reacted with modified proteins, the excess labeling molecules is rinsed away and fluorescence signals that reflect relative amounts of protein phosphorylation in different protein samples are measured. In some embodiments the beta-eliminated site is reacted with a linker that provides a reactive site for subsequent dye labeling.

Other methods are available to detect other posttranslational modifications of proteins by pre- or post-labeling on gels where protein expression ratios have been measured. Thus, protein multiplex methods can be extended for simultaneous monitoring of changes in phosphorylation, as well as the changes in protein levels and other posttranslational modifications of proteins.

The water solubility and fixed charges of many of the optical labeling molecules described herein provide low membrane permeability and low penetration into hydrophobic interiors of protein complexes and thus limit reaction to groups on the surface of proteins. Accordingly, the optical labeling molecules described herein can used to determine whether a particular protein is exposed to solvent. In some embodiments, a first optical labeling molecule is used to label exposed target proteins on the surfaces of cells, isolated organelles or isolated multiprotein complexes. The cell or organelle membranes or the multiprotein complex structure are then disrupted with detergents and/or chaotropic compounds and the interior groups labeled with a second, different optical labeling molecule. The sample is then separated by a method described above. Those proteins labeled with the first optical labeling molecule are proteins exposed to the surface of the cell, organelle or multiprotein complex. Those proteins labeled with the second optical labeling molecule are proteins that are not exposed to the surface of cell, organelle or multiprotein complex. In some embodiments, the labeled proteins are isolated and identified, as described previously.

In addition, as will be appreciated by those in the art, the optical labeling molecules described herein can be used in any standard application of optical labels. For example, the single proteins can analyzed or mixtures of proteins can be analyzed on 1D gels. A wide variety of techniques and applications in which the optical labeling molecules described herein are described in the 9^{th} edition of the Molecular Probes Catalog and references cited therein. Similarly, the optical labeling molecules described herein can be used in certain nucleic acid analyses such as gene expression and genotyping. The optical labeling molecules described herein can also be used as universal protein stains in 1D gels or in aptamer binding analysis.

In another embodiment, a set of at least two different optical labeling molecules of described herein for use in labeling at least two different target proteins in a sample, wherein a protein labeled with one of the optical labeling molecules does not exhibit an identical electrophoretic mobility pattern to a second protein labeled with a different optical labeling molecule. In yet another embodiment, each optical labeling molecule does not shift isoelectric point of said labeled protein.

In another embodiment, the present invention is directed to a method of differential analysis of proteins comprising covalently modifying different samples of proteins with different optical labeling molecules to form pluralities of differently labeled proteins; mixing the different samples of labeled proteins together to form a mixture; separating the proteins in the mixture via 2 dimensional (2D) gel electrophoresis to obtain a gel with separated differently labeled proteins; scanning the gels to provide optical images of the separated differently labeled proteins; matching the differently labeled protein images labeled with the differently optical labeling molecules; and simultaneously determining the changes in relative amounts of differently labeled proteins by correlating said changes with the strength of the optical images of the labeled proteins. In still another embodiment, the matching step is performed to adjust the images to compensate for differences in gel mobility among proteins labeled with different optical labeling molecules using gel pattern matching software. In yet another embodiment, the optical labeling molecule is a fluorescent dye and said gel is scanned with light excitation to provide fluorescent images of the differently colored optical labeling molecules. In these methods the optical labeling molecules comprise at least two optical labeling molecules described herein. Further, in the method the optical labeling molecule is cleaved from the target proteins after the determining step. In yet a further embodiment, the optical labeling molecule upon cleavage from the target protein leaves an isotopic tag attached to the target protein. In these methods the identities of the separated labeled proteins are determined by mass spectral techniques, and the relative amounts of the separated labeled proteins in the different samples are determined from the relative abundances of the isotopic tags by mass spectral techniques.

In a further embodiment, the method is used for the differential analysis of the proteins comprises at least one of the following analysis selected from the group consisting of relative amounts of protein, absolute amounts of protein, analysis of relative amounts of posttranslational modifications, analysis of relative levels of enzyme activities, analysis of protein levels, analysis of cell or organelle surface-exposed proteins and phosphorylation states of proteins.

In another embodiment, a method of labeling at least one target protein in a sample comprising covalently labeling at least one target protein with at least one optical labeling molecule according to the optical labeling molecules described herein.

In another embodiment, the method of labeling at least one target protein in at least two different samples comprising covalently labeling at least one target protein with at least one (or the first) optical labeling molecule of the present invention in one sample, wherein a protein labeled with one of the optical labeling molecules does not exhibit an identical electrophoretic mobility pattern to the same protein labeled with a different optical labeling molecule in a different sample. In still a further embodiment, more than one target protein in a plurality of target proteins in a sample are each covalently labeled with the same optical labeling molecule to form a plurality of labeled target proteins. Each optical labeling molecule does not shift the isoelectric point of said labeled protein. Additionally, the plurality of different labeled proteins are mixed and separated simultaneously prior to the determining the relative amounts of each of the different labeled proteins in the samples. In another embodiment, the present method further comprising simultaneously determining the changes in relative amounts of differently labeled proteins in at least the two samples by correlating said changes with the intensities of the optical images of labeled proteins. In a further embodiment of these methods, the different labeled proteins are separated by a method selected from the group consisting of 1 dimensional (1D) gel electrophoresis, 2 dimensional (2D) gel electrophoresis, capillary electrophoresis, 1 dimensional (1D) chromatography, 2 dimensional (2D) chromatography and 3 dimensional (3D) chromatography. In a still further embodiment, the optical labeling molecule is cleaved from the target proteins prior to the determining step. Additionally, the optical labeling molecule upon cleavage from the target protein leaves an isotopic tag attached to the target protein. In these methods, the identities of the separated labeled proteins are determined by mass spectral techniques, and the relative amounts of the separated labeled proteins in the different samples are determined from the relative abundances of the isotopic tags by mass spectral techniques.

The following examples serve to more fully describe the manner of making and/or using the above-described invention, as well as to set forth the best modes contemplated for carrying out various aspects of the invention. It is understood that these examples in no way serve to limit the true scope of this invention, but rather are presented for illustrative purposes. All references cited herein are hereby expressly incorporated by reference in their entirety.

### EXAMPLES

### Example 1

### Evaluation and Optimization of Labeling of Target Proteins from Different Types of Samples

The sensitivity of labeling to pH, buffer type, and common salts in the reaction medium is tested for different sample types, using parallel readout of the results of different conditions on ID electrophoresis and quantitation of labeled proteins with laser excited fluorescent gel scanning. Phosphate buffer is used near pH 7.4, a phosphate/borate mixture near pH 8, and borate or BICINE near pH 8.5 or 9.0. Tris buffers or other buffers with potentially reactive amines are best avoided. The best ratio of labeling to hydrolysis is near pH 8.5, unless SDS or other anionic detergent is used to solubilize the proteins and then a somewhat higher pH is favorable. The labeling rate of amino groups with the sulfo-succinamidyl or succinamidyl groups increases with pH, however at too high a pH the sulfo-succinamidyl or succinamidyl group hydrolyzes. Labeling kinetics are measured by quenching the labeling reactions at different times with excess glycine, taurine, hydroxylamine or low pH. Possible enhancement of labeling can be assessed for different samples in the presence of the detergents, urea, and thiourea used for IEF, using, 1D SDS gels and fluorescence emission as the readout. Saturation labeling of protein thiols can also be assessed in this manner.

After favorable pH and labeling times are established for samples from different organisms or tissues, experiments may be carried out to vary the optical labeling molecule/protein ratio during labeling. The approximate number of optical labeling molecules per labeled protein and the relative fluorescence of the optical labeling molecules on different labeled proteins is assessed, using on-line fluorescence and absorbance detection in HPLC gel filtration experiments. The HPLC gel filtration separates the free optical labeling molecule from the labeled proteins. Proteins used in such studies can be chosen to allow separation based on size by HPLC gel filtration. The amount of each protein added to the reaction mixture is known and the amount of 280 nm absorbance observed from the known amount of protein is determined in the HPLC on unlabeled and labeled samples. The stoichiometry of the optical labeling molecule to protein is determined from absorbance measurements of the dye moiety of the optical labeling on each protein peak and the relative extinction coefficients of the protein and the dye moiety. Fluorescence/absorbance ratios on each protein peak, relative to the free optical labeling molecule, allows detection of fluorescence quenching by the protein or by excessive numbers of optical labeling molecule/protein.

Such experiments also allow determination of the ratio of protein labeling to optical labeling molecule hydrolysis under different conditions, as it is desirable to minimize the remaining free optical labeling molecule for improved detection of low molecular weight proteins. The ratio of hydrolyzed and unreacted optical labeling molecule are determined on the free optical labeling molecule fraction by RP-HPLC. Too high an optical labeling molecule concentration during labeling might produce some dye fluorescence quenching by excessive protein labeling or produce inactive optical labeling molecule noncovalent dimers or even higher multimers from these particular optical labeling molecule. If optical labeling molecule dimerization occurs, it will be controlled by variation of labeling conditions. If necessary, more sterically-hindered tertiary amine groups (such as a t-butyl) can be substituted for the titratable group in the synthesis of the dye.

The strength of on-gel fluorescent signals is measured as a function of the number of optical labeling molecules per protein using gel filtration analysis of aliquots of the samples, where the labeling stoichiometry has been determined by gel filtration, as described above. It is not anticipated that the quenching of fluorescent signals will differ much in solution compared to gels, as a function of the number of optical labeling molecule/protein, except at the highest protein loadings on gels where fluorescence quenching may be observed. Such experiments establish the range of linearity of fluorescence signals and the dynamic range of detection of optical labeling molecule-labeled proteins on gels. Any differences in labeling of proteins in specific mixtures of proteins with different members of the optical labeling molecule sets, or families, can be detected by splitting identical protein mixtures, labeling each half of the sample with different optical labeling molecule, mixing the samples and detecting the fluorescence ratios for each band on 2D gels. Any departure from a constant ratio of fluorescence signals across bands on the gel would indicate differences in labeling, but this is not expected to be significant. If significant optical labeling molecule-dependent labeling is seen with some proteins, a labeling reversal experiment should be done routinely to allow correction for this effect in practical functional proteomics experiments.

The stability of dye binding to labeled proteins can be determined by centrifugal filtration to concentrate each protein peak from HPLC gel filtration, incubation of the purified, labeled proteins for various times (in the presence of sodium azide and protease inhibitors) and measuring any loss of labeling by rerunning on gel filtration. The UV-reversible linkages in some of the compounds require protection from fluorescent light during experimental manipulation for highest stability, and sample tubes must be wrapped in opaque material and manipulated under dim incandescent light.

### Example 2

### Effect of Optical Labeling Molecule on Protein Solubility and Two-Dimensional Gel Electrophoresis Mobility

The effect of the optical labeling molecule on protein solubility and 2D gel mobility is assessed using fluorescent signals and radioactive labeling of standard proteins. The solubilities of labeled proteins can be assessed by running them on IEF (isoelectric focusing) and 2D (two-dimensional) electrophoresis to assess any changes of retention of proteins on the IEF strips before and after labeling. Retention of protein on the IEF strips and poor transfer into the second dimension is often found in 2D electrophoresis if sample loadings are too high or if solubilization conditions are inadequate. Fluorescent signals of labeled proteins retained on IEF strips provide semi-quantitative measurements of limited solubility since the strong signals can exceed the linear range. The use of the optical labeling molecules described herein will lead to substantial protein solubility increases compared to the unlabeled protein samples. To verify this phenomenon, radioactively labeled standard proteins and complex mixtures of proteins from cells are used for assessment of any labeling induced gel mobility shifts (see below) and these same radioactive proteins will be useful for quantitative solubility assessments. Phosphorimaging of the 2D gels, and any protein residues on the IEF strips, provides a quantitative measure of insoluble proteins remaining on the LEE strips, relative to the radioactivity on the second dimension.

Two methods of radioactive labeling of the standard proteins are used. N-acetyl labeling with tritiated acetic anhydride at near neutral pH largely couple to N-terminal groups. Excess acetic anhydride will be removed by HPLC gel filtration, followed by fluorescent dye labeling of the epsilon amino groups of lysine at elevated pH (e.g., 8.5). An alternative method of radioactive labeling first reduces protein sulfhydryl groups with tributylphosphine (TBP), tricarboxyethyl phosphine (TCEP), or other trisubstituted phosphine compound. The sulfhydryl groups are then labeled with radioactive iodoacetamide and the amino groups labeled with dyes.

2D gels are run on the radioactively tagged and fluorescently labeled proteins after low (substoichiometric), medium (one or two optical labeling molecules per protein) and high optical labeling molecules labeling (many optical labeling molecules per protein). Gels are scanned for fluorescence and the location of radioactive spots will be measured by phosphorimaging on a Fluorescent Gel Scanner and Phosphoimager. The radioactivity shows the position of proteins that are not dye labeled, as well as the dye labeled proteins. Thus, any optical labeling molecule-induced shifts in protein patterns is detected and monitored by comparing radioactivity patterns to fluorescence patterns. An expected reduction of shifts is assessed using the optical labeling molecules with titratable groups. The dyes with titratable amine groups are especially valuable in the high pH range from 9-12. Commercial IEF strips are now available from Pharmacia up to pH=11 and if strips up to pH=12 are not commercially available, the needed strips may be prepared following procedures known in the art (Gorg et al., (1999) Electrophoresis 20: 712-717; Gorg, (1999) Methods Mol. Biol. 112, 197-209; Gorg et al., (2000) Electrophoresis 21, 1037-1053). The larger the multiplicity of optical labeling molecules labeling on target proteins, the larger the fluorescent signals (up to the point where fluorescence quenching becomes a problem). Thus, labeling conditions can be optimized for maximum sensitivity, consistent with minimal mobility shifts for mixtures of proteins from particular organisms or tissues and thiol saturation labeling can be used to maximize sensitivity.

With two (or multiple) color ratio recording of fluorescent signals, the information content as to which proteins are changing with physiological stimulus is insensitive to optical labeling molecule-induced shifts as long as the shifts are the same or very similar for the different dyes. However, increased complexity or spot distortion would occur if labeling shifted the gel mobility with increasing number of optical labeling molecules bound/protein. If labeled protein spots are resolved from other proteins then the fluorescence ratios will still contain reliable information on the relative expression of proteins under different physiological conditions. Thus, any significant shifts with labeling will favor increased reliance on narrow pH range IEF gels to spread proteins over 1 or 2 unit pH range. Optical labeling molecule-induced shifts are not expected to be very large due to the modest resolution of 2D gels. A tradeoff between minimum complexity and lower sensitivity with sub-stoichiometric labeling, to possibly more spot complexity and highest sensitivity with high optical labeling molecule labeling will be under experimental control.

### Example 3

### Testing of the Protein Pre-Labeling Methods on Standard Proteins

A very large range of protein abundance/concentration is found in cells, tissues and bodily fluids. Increased dynamic range of protein measurement can be obtained by labeling samples at more than one level of dye multiplicity and scanning gels at several different photomultiplier amplifications. After the desirable conditions for different multiplicity of optical labeling molecule labeling are established for particular protein mixtures, the detection limit and linearity of the fluorescence signal vs. amount of protein loading can be determined. These experiments can be carried out at low labeling multiplicity, medium multiplicity and high multiplicity of optical labeling molecule labeling that is found to be useful in prior experiments and can also determine the dynamic range for the method and the scanner in practice. A dilution series of standard proteins labeled with the optical labeling molecules is made and the different dilutions run on different lanes of ID gels.

Similar experiments can be carried out with two and three or several different optical labeling molecules using identical standard protein mixtures. In multiple color optical labeling molecule experiments, dye cross talk and multiplex sensitivity is determined, using constant amounts of one or two of the labeled protein mixtures (at a relatively high level) and varying the amount of proteins labeled with a second or third optical labeling molecule in steps from the detection limit to very high levels. The degree of crosstalk between the two main groups of optical labeling molecule investigated is extremely low due to the essentially non-existent direct excitation of the partner dyes by the lasers to be used. Double-label pairs with minimum cross-talk are dyes excited with the 488 nm laser-paired with dyes efficiently excited with the 633 nm laser.

A third optical labeling molecule excited efficiently with the 532 mm laser, with only modest cross talk expected with the other dyes. The degree of crosstalk is determined by comparing gels from a standard curve of protein fluorescence on a dilution series, using a single optical labeling molecule, to the same dilution series in the presence of a constant, high level of proteins labeled with a second optical labeling molecule. Any preference of optical labeling molecule for different proteins is determined by labeling protein mixtures separately with the different optical labeling molecules, mixing the two or three different labeled proteins in the same amounts, running electrophoretic separations and determining the fluorescence color ratios.

### Example 4

### Recovery of Proteins from 2D Gels and Efficiency of Removal of Optical Labeling Molecule

The recovery of proteins from 2D gels and efficiency of removal of the optical labeling molecule is assessed and optimized using radioactively labeled proteins with and without the optical labeling molecule. Initial experiments are carried out in aqueous solution on glycine-quenched dyes to test the amount and type of UV irradiation needed to remove the reversible cleavable group efficiently, using RP-FPLC to analyze the products. Known amounts of labeled standard proteins are run in duplicates. Fluorescence and phosphoimager scanning can be used to confirm the dilution series. Consistent-sized gel circles are punched out of the gel, frozen in liquid nitrogen and the gel pieces powdered with a stainless steel rod in microfuge tubes. One of the duplicate samples is counted for radioactivity and the other is freeze-dried and then rehydrated in a buffer containing Promega autolysis-resistant trypsin, (+/−TCEP and IAA to enhance recovery of cysteine-containing peptides). Dye labeled and control samples are treated with UV (365 nm mercury lamp) to remove the reversible optical label molecule linkage. After incubation (24-48 hours) gel pieces are extracted with 50% acetonitrile and the supernatant harvested by centrifugal filtration using a filter that is resistant to acetonitrile (e.g., Millipore Biomax) to retain the gel fragments. The extraction is repeated once or more with acetonitrile and the extracts are counted to determine the recovery of peptide radioactivity. Control proteins with no labels are hydrolyzed in solution with trypsin in H₂O¹⁸ to mark the trypsin cleavage sites with 0¹⁸ substitution (Shevchenko et al., (2001) Anal. Biochem 296, 279-283). Aliquots of the 0¹⁸-labeled peptides are added to the extraction steps and the ratios of 0¹⁶ peptides to 0¹⁸ peptides monitored by mass spectrometry to determine the percentage of recovery of peptides from the protein. The peptides are run on MALDI and ESI/MS/MS to determine peptide recovery +/−UV treatment to remove the dye labels, using 0¹⁸ internal standards. Standard acrylamide gels and meltable Proto-Preps system gels (National Diagnostics) will be compared. Protocols for efficient protein digestion and peptide recovery will be optimized to maximize the conditions for effective protein identification using mass spectral analysis. 0.1% octyl glucoside may be included to improve recovery of tryptic peptides from in-gel digests (Mann et al., (2001) Annu. Rev. Biochem. 10, 437-473).

### Example 5

### Testing of the Optical Labeling Molecules on Total Bacterial Proteins

The properties of optical labeling molecules can be evaluated on the complex protein mixture in the total protein complement of an organism. For example, the hyperthermophilic archeabacterium, Sulfolobus solfararicus, can be used to evaluate optical labeling molecules.

An advantage to the use of a microorganism for testing and evaluation of proteomic methodology is that all the proteins in the microorganisms can easily be radioactively labeled, using radioactive sulfur⁻³⁵ in the growth medium. Radioactive labeling provides tremendous advantages for assessment of protein recovery from gels and any label-induced gel mobility shifts. Essentially the same techniques are used for analysis of the total Sulfolobus proteins as was described above. Sulfolobus provides a wide range (about 3,316 proteins in the genome) of proteins with a much greater variety of characteristics, than possessed by standard protein mixtures (discussed in earlier sections). In particular, there is the opportunity to discover any dye-specific labeling preferences in the wide range of Sulfolobus proteins using simple dye cross-over labeling experiments. Comparison of radioactivity and dye labeling are used to detect any dye labeling-induced shifts on complex protein mixtures from Sulfolobus. Protein spots are cut out of the gel, the dye label is removed by UV irradiation (365 or 308 nm), the proteins digested with trypsin in the presence of octyl glucoside to enhance recovery (Katayama et al., (2001) Rapid Comm. Mass Spectrom. 15, 1416-1421), peptides are extracted and submitted to mass spectral analysis using the best procedures available (Gygi et al., (2000) Curr. Opin. Chem. Biol. 4: 489-494; Loo et al., (1999) Electrophoresis 20, 743-748, Kraft et al., (2001) Anal. Biochem. 292, 76-86). For example, nano-spray and tandem mass spectral techniques can be used as a method to identify proteins and posttranslational modifications.

### Example 6

### Testing of the Effect of Higher Dye Labeling Molecules on Number of Proteins Detected

As pointed out in the background, existing fluorescent dyes for detecting protein changes on 2D gels have limited sensitivity because they are rather oily molecules and decrease the solubility of dye-labeled proteins. These solubility limitations restrict the amount of dye that can be added to the proteins before proteins are lost to analysis by precipitation. In this example we describe how by using the highly water soluble zwitterionic optical labeling molecules disclosed that heavier dye labeling can be accomplished and this greatly increases number of proteins that can be detected in a given sample. The example described here uses E. coli cytosolic proteins which are a complex mixture that is a commercially available test sample.

Any type of simple or complex protein mixture can be labeled and analyzed and differences in the amounts of the different proteins in complex mixtures can be determined. In this case a single colored optical labeling molecule (e.g. 43) is used at different levels of labeling for different identical samples and the number of proteins that can be detected is shown to increase greatly with heavier dye labeling.

In this example, freeze-dried, commercial (BioRad 163-2110) E. coli cytosolic proteins are dissolved in TU4 buffer (7M Urea, 2M Thiourea, 30 mM Bicine pH 8.5, 4% CHAPS plus a 1:100 dilution of protease inhibitor cocktail (Complete Mini, EDTA-free Roche # 11836170001)) at a ratio 10 μl per mg of wet cell pellet.

Protein extracts were diluted to a final concentration of 5 mg/ml in TU4 buffer. Ten microliters containing 50 μg of protein was labeled with 1× (400 pmol) or 5× (2 nmol), 10× (4 nmol), or 20× (8 nmol) optical labeling molecule (e.g. 43) diluted in DMF (final concentration of 10% DMF) for 30 minutes at on ice tin the dark. Excess dye was quenched with a 100× molar excess of Lysine pH 8.5 for 30 minutes at on ice in the dark.

Rehydration buffer was prepared by adding 1% carrier ampholytes and 15 mg/ml Destreak (GE Healthcare) Reagent to TU4 buffer. Samples were diluted in rehydration buffer and added to wells for strip rehydration. IEF strips were rehydrated for 14 hours at 50 volts and were focused with a maximum of 50 μA/strip, using the following IEF program: Step 300 volts 3 hours, Gradient 3500 volts, 6 hours, Step 3500 volts, 6 hours, Gradient 5000 volts, 3 hours, and Step 5000 volts, 6 hours. Strips were equilibrated in 5 mls equilibration buffer (6M Urea, 4% SDS, 30% Glycerol, 50 mM Tris pH 8.8) containing 130 mM DTT for 15 minutes with gentle rocking. Excess buffer was removed by blotting before equilibration in equilibration buffer containing IAA. Strips were then equilibrated in 5 ml equilibration buffer containing 270 mM IAA for 15 minutes with gentle rocking. Excess buffer was blotted off and strips were briefly rinsed with 1× running buffer before loading onto gels. Equilibrated strips were loaded onto 11% polyacrylamide gels, and overlayed with 0.5% agarose containing bromophenol blue in 1× running buffer. Gels were run using the following program.: Step 1—1000 volts 2 hours 5 mA/gel, 1000 volts 12 hours 10 mA/gel, 1000 volts 6 hours 20 mA/gel. Gels were scanned on a Typhoon Trio, using the 532 nm laser and filters appropriate for optical labeling molecule (43) employed in this experiment. In these particular experiments no image matching was needed since single optical labeling molecule; i.e., (43) was used.

More spots are clearly seen visually with higher optical labeling molecule (43) when the labeling is compared at 1×, 5×, 10× and 20× the recommended labeling levels for commercial DIGE dyes from GE Healthcare. The number of protein spots detected on a 24 cm. 3-10 ranged from 1,245, 3,252, 4,005, and 4,825 with the increasing labeling of 1×, 5×, 10× and 20× labeling. This was not due to spot doubling by adding dyes since the patterns were superimposible. The biggest change was between 1× and 5×, where the number of spots increased by 2.6 fold, which approaches the number of genes in E. coli. The increasing number of spots with higher labeling may be revealing posttranslationally modified forms of the proteins.

The detectability of proteins at the higher multiplicity of labeling is limited also by the separation of the proteins in the first dimension and the dynamic range of the detector in the scanner, both of which have been increased, the first by using narrower range IEF strips and the second by using an image processing technique to merge images obtained with lower and higher sensitivity, as described in the specifications. The maximum number of spots that can be resolved is about 5,640, which implies that all the proteins and isoforms present may be resolved and that there are approximately three postranslational modifications per protein in the E. coli cytosol.

### Example 7

### Differential Analysis of Developing Rat Brain Proteins Raised on High and Low Levels of Docosahexaenoic Acid

Docosahexaenoic acid (DHA) is a key member of an essential fatty acid family (the omega-3 family), that is rich in ocean fish. DHA is of supreme importance for developing optimium learning, memory and low anxiety in rodent, monkey and human brains. The mechanism of these beneficial effects is not known and high sensitivity global proteomics was used to investigate this mechanism as set forth below.

Sample preparation—Each forebrain was ground using a pestle and mortar, previously brought to liquid nitrogen temperature in a sealed plastic bag with positive dry nitrogen gas pressure. After grinding of the tissue at liquid nitrogen temperature, 10 μL/μg sample of cell lysis buffer containing 20 mM Bicine, 5 mM magnesium acetate, 0.5% Nonidet P-40 and Roche Complete protease inhibitor-mini EDTA free, were added to about a 100 mg sample and mixed well in a 2 ml microcentrifuge tube. Sample fractionation—Crude nuclear fraction (P1) was removed by sedimentation at 1000×g for 10 min at room temp. The supernatant (S1) was removed and was ultra-centrifuged at 100,000×g at 4° C. for an hour in 2 ml polycarbonate tubes in a swinging bucket rotor. The cytosolic fraction S2 was separated. The pellet (P2), which contained the membrane organelle fraction, was re-suspend in lysis buffer and washed once by centrifugation under the same conditions. P2 was solubilized in a buffer containing 6M urea, 2M thiourea, 2% CHAPS, 2% ASB-14 (amidosulfobetain-14), 20 mM tris and 5 mM magnesium acetate, at pH 8.5. S2, the cytosolic fraction, was precipitated, using a GE healthcare “2-D Clean-up Kit”, and re-suspended at the same solubilization buffer as P2. Protein concentrations of the cytosolic and membrane organelle fractions were assessed, using the Bio-Rad RCDC assay.

Each brain fraction sample was paired with the same fraction of brain from the other diet group (n=4 in each group) for dye labeling and further analysis. The experiment was designed to have four technical gel repetitions for each animal pair, consisting of two replicas with control samples labeled with optical labeling molecule (43), and DHA enriched samples labeled with optical labeling molecule (54). The two other replicas were reciprocally labeled with the different colored optical labeling molecules to test for and account for any differential dye labeling effects.

Protein labeling reactions—were carried out, as recommended by GE Healthcare for DIGE dyes. Briefly: 1 μL (400 pmole) of one of the optical labeling molecules (43), (54), or (236) in dimethylformamide were added, respectively, to 50 μg (in about 10 μL at pH 8.5) of proteins of each of the two sample diet groups or an internal standard combining 25 μg of each of the two diet group samples. The reactions took place in the dark and on ice for 45 min, and the dye reactions were quenched by addition of 1 μl, of 10 mM Lysine and incubation for 10 min. The differentially labeled n-3 adequate, DHA enriched and internal standard samples were pooled and brought to a final volume of 350 μL in solubilization buffer. Bio-Rad carrier-ampholytes 3-10 (final concentration 0.2% w/v) and hydroxyethyldisulfide (HED, 1% v/v final conc') were added.
2 dimensional gel electrophoresis (all carried out in the dark except for handling the strips and gels in between steps)—1^{st} dimension separation—the sample was spread in a lane of a Bio-Rad Protean isoelectric focusing (IEF) cell tray, an 18 cm 3-10 non-linear IPG strip, or 3-7, or 6-11 IEF strip was put on top of it, covered with mineral oil and allowed to passively re-hydrate for one hr. Paper wicks moistened with water were placed between the strip ends and the electrodes, followed by 14 hrs of active re-hydration at 50 volts per strip at 20° C. The IPG strips were then transferred to an Ettan IPGphor and covered with mineral oil for monitored iso-electric focusing as follows, 3:30 hrs 300V, 2:30 hrs 1000V, 2:30 hrs 2500V and 7:30 hrs 3500V. Focused IPG strips were kept at −80° C. until further processing.
Equilibration—IPG strips were manually shaken every five min for 15 min in 5 mL equilibration buffer (6M Urea, 375 mM Tris, 20% Glycerol and 2% SDS, pH 8.8) with 32 mM DTT, and then transferred to 15 min shaking in 5 mL equilibration buffer containing 216 mM IAA. Excess equilibration buffer was then washed from the strips with 1×SDS running buffer. 2^{nd} dimension—IPG's were loaded onto 18 cm 11% non-gradient polyacrylamide gels, sealed with 0.5% agarose containing Bromophenol blue (BPB), and run for 2 hrs at 5 mA/gel, followed by approximately 9 hrs at 20 mA/gel, until the BPB dye running front reached the end of the gel.

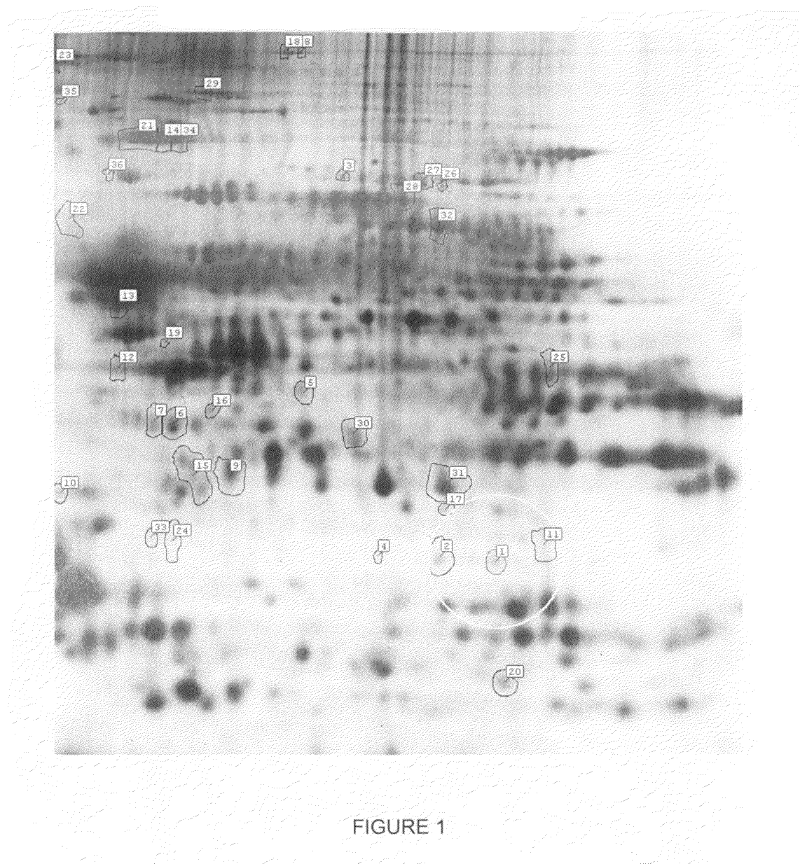

Imaging and analysis—Images were obtained using a Typhoon Trio imager, which was previously optimized for imaging the three optical labeling molecules. Each gel was scanned using three different fluorescence channels within six hours from the end of 2nd dimension run. Because the different colored optical labeling molecule labeled proteins do not have the same mobility in the second dimension, the different-colored images of the same gels were first matched using Progenesis (NonLinear Dynamics), a program originally designed to match the images of different gels, which can differ much more than the different-colored images of the same gels. Other image matching programs such as PDQuest can also be used. Allowing for the matching step is very important because it is very difficult to adjust the structures of the optical labeling molecules to give the same mobilities in the second dimension of the 2D gels for a wide range of different colors and protein coupling chemistries that are desirable to use. The experimental data supports the lack of equal mobility in the second dimension of the different colored optical labeling molecule-labeled proteins before matching with the computer program. After matching the images of the different optical labeling molecules, there is excellent matching of the imaging and accurate ratios of the image intensities can be determined. Once the different-color images are adjusted by matching then the ratios of image colors can be used to locate proteins that differ between the different experimental treatments. If some spots are remain colored after matching this indicates higher or lower amounts of those proteins in the compared samples. Representative 2D gel image of cytosolic brain fraction in shown in FIG. 1. The different-colored images were matched/warped to achieve exact pixel alignment for the different-colored images. The image is shown in black and white because B&W images have more dynamic range than color images (which are limited to 8 bit resolution, which equals 256 image levels). First dimension—18 cm, pH 3-10 isoelectric focusing. 2^{nd} dimension—11% non-gradient gel, SDS-PAGE. The circled spots—are ranked by signification of differential protein expression between the two samples.

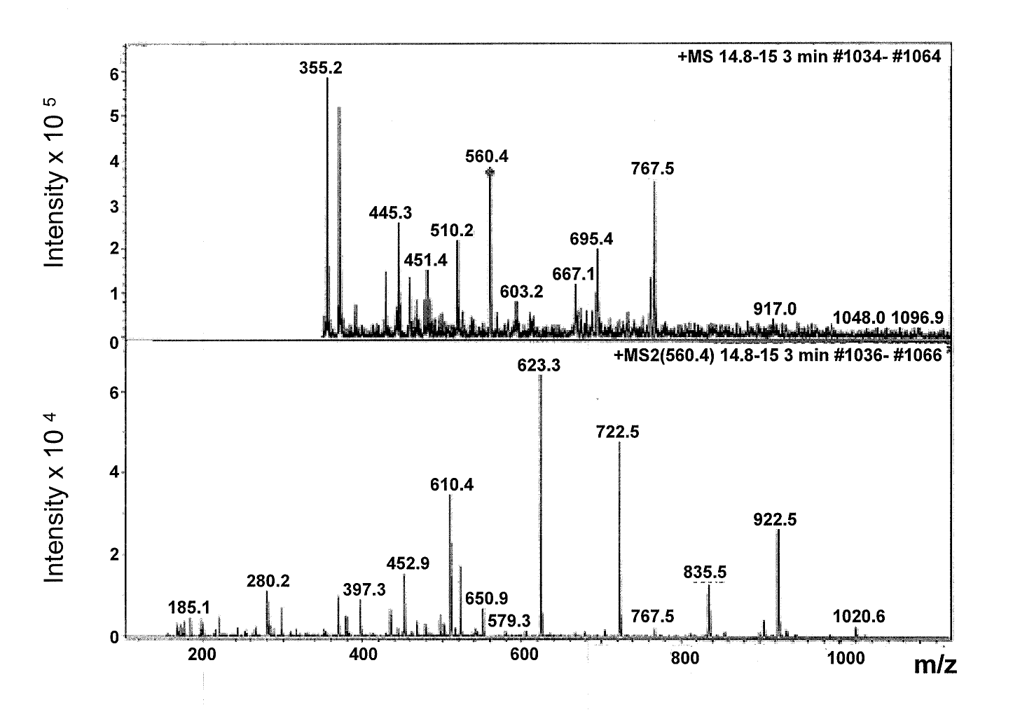

The proteins that show statistically significant changes are cut out of the gels, digested and analyzed by mass spectrometry to identify the proteins and the protein posttranslational modifications of interest. We found 8 spots of most interest (P value≦0.05) in this particular experiment. The top two ranking spots (spots 1 & 2), showed approximately the same molecular weight and half a pI unit difference, and reported up-regulation of 2.5 and down-regulation of 1.7 in the DHA enriched diet, compared with the adequate control diet, respectively. Those spots were manually picked from an analytical gel (a representative gel, containing 200 μg protein pooled from all samples, that followed the same 2D separation protocol described previously, and was fixed in 10% Methanol 7% acetic acid, and stained with SyproRuby). After dehydration in a SpeedVac, trypsin (0.5 μg) was add, and the gel pieces were covered with 10 mM ammonium-bicarbonate, 10% acetonitrile buffer and incubated overnight at 37° C. After the in-gel digestion, the peptides were extracted with 0.1% trifluoracetic acid (TFA), 50% acetonitrile buffer, concentrated by SpeedVac and subjected to mass spectrometry analysis by Agilent ChipLC XCT Ultra ion trap coupled to an Agilent 1100 series nanoflow HPLC. The spectrum was scanned for MS and MS/MS ions (FIG. 2) and the data was analyzed using the Mascot online search engine (matrixscience.com), searching the NCBI non-redundant Rattus data base.

The highest up and down regulated proteins were both identified as Glutathione S Transferase Omega 1—GSTO1 (MOWSE scores 133 and 131 respectively). The predicted molecular weight and pI values generally agree with the experimental MW and pI, although, both proteins appear to be ˜3.5 KDa heavier than predicted. This slower than predicted migration on SDS gels was also observed by Board et al (2000), who suggested that GSTO1 migrates anomalously in reducing SDS-PAGE. Also, spot 1 was shifted about half a pH unit from the predicted pI to the basic region, presumably due to being posttranslationally modified. The other spots of interest and the change in modification between the two GSTO-1 isoforms are under investigation. Expanded IEF and means to more efficiently recover the proteins and the peptides is being pursued.

### Example 8

### Multiplex Detection of Phosphorylation

Phosphorylation is one of the most common posttranslational modifications in cellular regulation, but because of the labile nature of this modification, phosphorylation is difficult to detect by mass spectrometry. Some of the Trk receptor isoforms are phosphorylated and there is evidence that several signaling cascades are activated (Patapoutian et al., Curr Opin Neurobiol. 2001 June; 11(3):272-80). In addition to the methods of detecting the presence or absence of proteins, or quantity of protein, with fluorescence detection, multiplex detection of phosphorylation can be performed examining all the proteins on the same sample as described previously and below.

The dorsal root ganglia (DRG) cells are cultured as described (Garner et al., (1994) Neuron 13, 457-472), unstimulated cells are labeled with ³³P phosphate and growth factor stimulated cells are labeled with ³²P phosphate. After suitable incubation the two cell samples are extracted. The ³³P-labeled extracts are reacted with a first optical labeling molecule and the ³²P-labeled extracts are reacted with a second different optical labeling molecule. The first and the second optical labeling molecules are chosen from the same set of optical labeling molecules so that the optical signal is different but the physical characteristics are similar. The labeled extracts are mixed together, run on 2D gels and laser scanned for the protein expression ratios between the stimulated and unstimulated cells. In addition, two phosphoimager image plates are exposed simultaneously on two sides of the same gel, one phosphoimager plate directly on the gel and the other having a 1 mil thickness of copper foil in front of the phosphoimager plate (Bossinger et al., (1979) J. Biol. Chem., 254, 7986-7998; Johnston et al., (1990) Electrophoresis, 11, 355-360; Pickett et al., (1991) Molecular Dynamics Application Note). The directly exposed P1 plate registers the sum of both isotopes, whereas the copper foil-filtered phosphoimager image almost entirely blocks the ³¹P, whereas barely attenuating the signals from the ³²P. The results of these studies will be compared to direct dye staining of the serine and threonine phosphorylated proteins using beta-elimination of the phosphates by base treatment of the gels after fluorescent and phosphoimager scanning or after transfer of proteins to PVDF membranes and staining of the beta-eliminated sites with high sensitivity fluorescent dyes. Thus, the multiplex methods of the invention can be extended for with simultaneous monitoring of changes in phosphorylation, as well as the changes in the level and posttranslational modification of the proteins associated with function.

### Example 9

### Zdye Labeling of Phosphoproteins

Reduction and alkylation of proteins. Proteins are dissolved in 100 ul of 8M urea, 5% 3-((cholamidopropyl)-dimethylammonio)-1-propanesulfonate (CHAPS; Sigma-Aldrich, Co. St. Louis, Mo.), 30 mM Bicine pH 8.5. With the addition of 0.5 ul of TEP (triethyl phosphine, final conc. 34 mM), the solution was maintained at room temperature (RT) for at least 1 hr. Acrylamide was added to the solution to final concentration of 170 mM. The alkylation reaction was terminated after 2 hr incubation at RT. Alternative thiol alkylation reagents (such at 2-vinyl pyridine or 4-vinyl pyridine) can be used at appropriate concentrations. A total 400 ul of ice-cold acetone was added, after 1 hr incubation at −20° C., proteins were pelleted by centrifuging at 14,000×g for 5 min.

Beta-elimination and Michael addition of phosphorylated proteins. Sulfhydryl-protected proteins (0.05-1 mg) were dissolved in 130 μl of 5% CHAPS solution. A 20 μl aliquot of 1,2-ethanedithiol (EDT; Sigma-Aldrich) was diluted into 50 μl ethanol. To this EDT solution 50 μl of 150 mM Ba(OH)₂ and 250 μl acetonitrile were added, and finally the dissolved protein solution was added. This reaction solution was stirred at RT for 4 h. The reaction was terminated with 75 ul of 200 mM acetic acid, the unreacted EDT was extracted using 3×500 ul of chloroform. The aqueous layer was harvested and 800 ul of ice-cold acetone was added and the solution kept at −20° C. for 1 h and centrifuged at 14,000 g for 5 min. The pellet was washed with ethanol, and redissolved in 20 μl of 8 M urea, 0.5% CHAPS, 0.1 M sodium phosphate buffer (pH 6.8).

Zdye labeling. Maleimide Zdye (500 pmole per 50 ug protein) was added to the protein solution and incubated at RT for 30 min. The protein solution was further treated with 5 mM of Tris(2-carboxy ethylphosphine) hydrochloride (TCEP; Pierce) for 30 min and a second aliquot of maleimide Zdye was added and incubated for another 30 min. Unreacted Zdye was quenched by adding 1 ul of 2-mercaptoethanol.

Gel electrophoresis detection of the relative amounts of phosphoproteins in different protein mixtures. Different-colored maleimide Zdyes can be used for labeling different protein samples, the labeled samples mixed, separated on 1D or 2D gels, and the positions of the phosphoproteins and the relative amounts of the phosphoproteins determined by laser scanning of the gels, to detect the relative amounts of the different Zdye-labeled phosphoproteins. A third color of maleimide Zdye can be used to label a mixture of the proteins to be analyzed after reduction and alkylation to detect any residual protein thiol reactivity that would otherwise be mistaken for the occurrence of phosphogroups on the proteins of interest.

### Example 10

### Synthesis of Optical Labelling Molecules

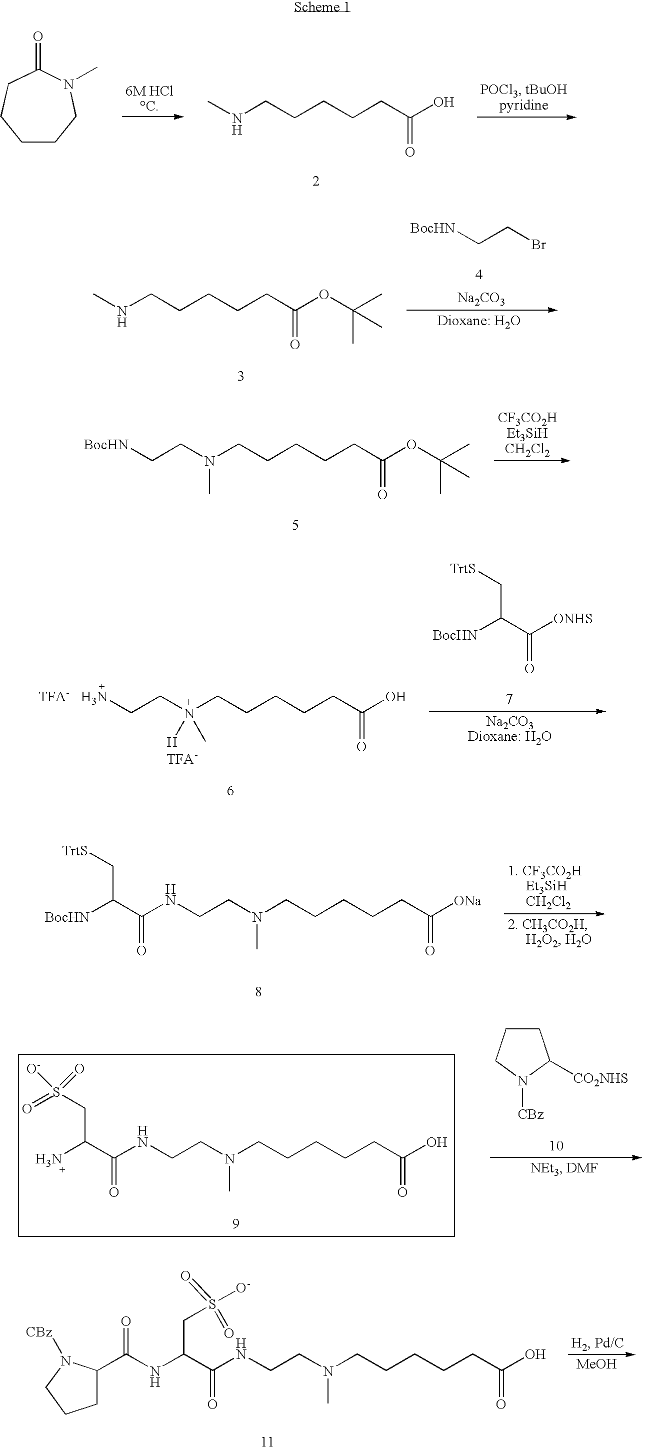

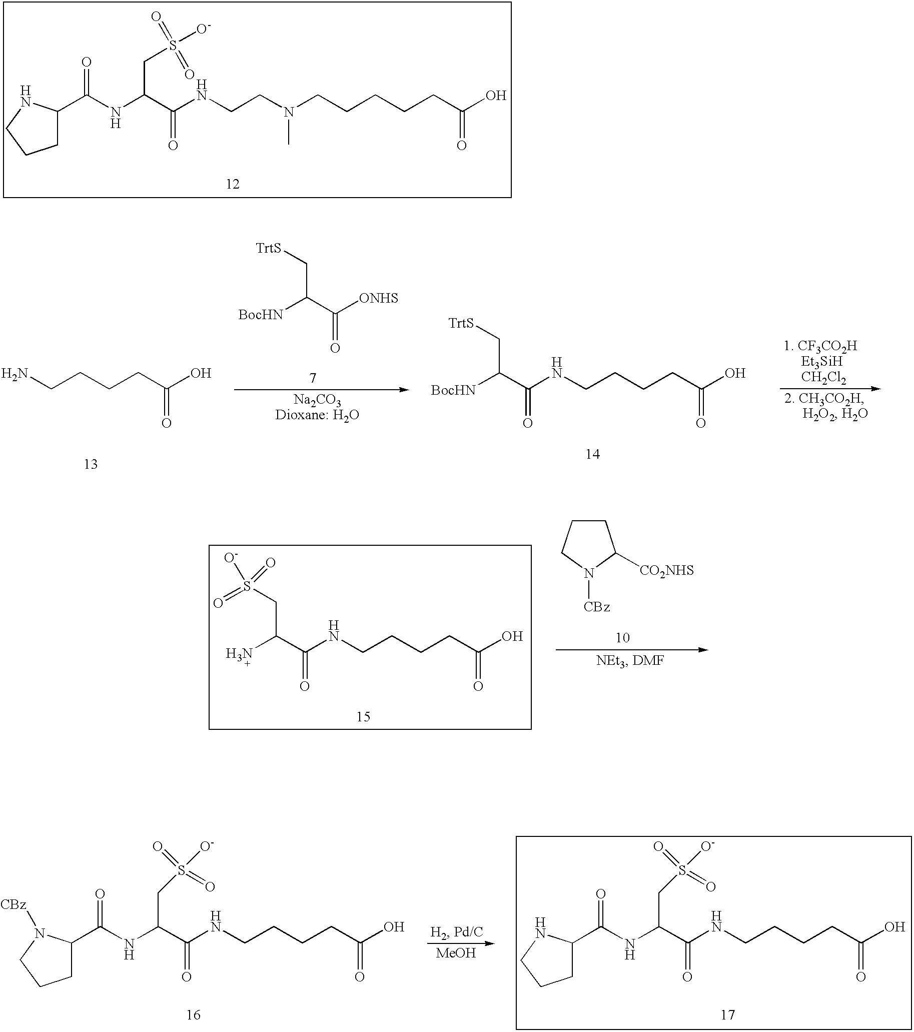

5-(methylamino)pentanoic acid (2): 6 M HCl (100 mL) was added to a flask containing N-methylcaprolactam (1) (15 g, 0.12 mmol) and the resulting solution was refluxed for 24 hours. After cooling, the solvent was removed by vacuum and benzene (200 mL) was added followed by refluxing into a Dean Stark trap. After 6 hours, the solution was cooled and the solvent was removed by vacuum to yield 17.1 g product (100%). ¹H NMR (500 MHz, D₂O) δ 2.9 (t, 2H, J=12 Hz), 2.56 (s, 3H), 2.26 (t, 2H, J=1120), 1.50 (m, 4H), 1.27 (m, 2H).

tert-Butyl-6-(methylamino)hexanoate (3): POCl₃ (0.981 mL, 10.6 mmol) was added to a solution of 5-(methylamino)pentanoic acid (2) (958 mg, 5.29 mmol), anhydrous pyridine (0.856 mL, 10.6 mmol) and t-BuOH (2.56 mL, 27 mmol) in anhydrous DCM (16 mL). Stirring was continued for 21 hours. The mixture was poured in brine and partitioned with DCM. The organic phase was washed an additional time with brine, twice with aqueous Na₂CO₃, once with water and finally with brine again. The organic extract was dried over Mg₂SO₄ and the solvent was removed in vacuo. The residue was purified by flash chromatography on Et₃N deactivated DAVISIL (10% MeOH; 90% DCM) to yield a yellowish oil (230 mg, 1.14 mmol, 22%); ¹H NMR (500 MHz, CDCl₃) δ 1.35-1.42 (m, 2H), 1.41 (s, 9H), 1.59 (p, 2H, J=7.5 Hz) 1.85 (p, 2H, J=7.5 Hz), 2.20 (t, 2H, J=7.5 Hz) 2.65 (s, 3H), 2.91 (t, 2H, J=7.5 Hz); ¹³C NMR (125 MHz, CDCl₃) δ 24.51, 25.75, 26.25, 28.27, 33.01, 35.28, 49.34, 80.39, 172.81; HRMS-ES (m/z): [M+H] calcd for C₁₁H₂₄NO₂⁺ 202.1807. found 202.1804.

### tert-Butyl-2-(N-methyl-N-(6-(tert-butoxycarbonyl)hexylamino))ethylcarbamate (5)

tert-Butyl-6-(methylamino)hexanoate (3) (205 mg, 1.02 mmol), tert-butyl 2-bromoethylcarbamate (4) (202 mg, 0.903 mmol) and Na₂CO₃ (316 mg, 2.97 mmol) was dissolved in a mixture of H₂O (1.8 mL) and 1,4-dioxane (1.8 mL). The solution was stirred at 80° C. for 3 hours. The mixture was allowed to cool to room temperature and the solvents were removed in vacuo. The resulting solid was partitioned between H₂O and DCM, and the aqueous phase was extracted with DCM (×3). The combined organic extracts were dried over Mg₂SO₄ and the solvent was removed in vacuo to yield the crude product which was used in the next step without further purification (270 mg, 0.785 mmol, 77%).

6-(N-(2-aminoethyl)-N-methylamino)hexanoic acid, bishydrotrifluoroacetate (6): Et₃SiH (1.44 mL, 9.04 mmol) was added to a solution of tert-Butyl-2-(N-methyl-N-(5-(tert-butoxycarbonyl)pentylamino))ethylcarbamate (5) (1.44 g, 4.19 mmol) in TFA (10 mL) and DCM (10 mL), and the mixture was stirred at room temperature under argon for 1 hour. Solvents were removed in vacuo and the residue partitioned between H₂O and Et₂O. The aqueous phase was evaporated in vacuo to yield the crude product which was used in the next step without further purification.

Sodium 6-(N-methyl-N-(2-(2-tert-butoxycarbonylamino-3-tritylsulfanyl-propionamido)ethyl)amino)hexanoate (8): A solution of 6-(N-(2-aminoethyl)-N-methylamino)hexanoic acid, bishydrotrifluoroacetate (6) (4.19 mmol), Boc-Cys(Trt)-OSu (7) (4.69 g, 8.37 mmol) and Na₂CO₃ (1.77 g, 16.7 mmol) in H₂O (40 mL) and 1,4-dioxane (40 mL) was stirred at room temperature for 48 hours. The 1,4-dioxane was removed in vacuo and the resulting solution was extracted with EtOAc. The EtOAc extract was dried over Na₂SO₄ and the solvent was removed in vacuo. The residue was purified by flash chromatography on DAVISIL (10 to 20% MeOH in DCM) to yield a white solid (2 g, 3.07 mmol, 73% over 2 steps): ¹H NMR (500 MHz, CD₃OD) δ 1.36 (p, 2H, J=7.5 Hz), 1.44 (s, 9H), 1.62 (p, 2H, J=7.5 Hz), 2.21 (t, 2H, J=7.5 Hz), 2.45-2.57 (m, 2H), 2.64 (s, 3H), 2.87 (t, 2H, J=7.5 Hz), 2.96 (br, 2H), 337-3.51 (m, 2H), 3.92 (t, 1H, J=6.5 Hz), 7.23 (t, 3H, J=7.5 Hz), 7.29 (t, 6H, J=7.5 Hz), 7.37 (d, 6H, J=7.5 Hz); ¹³C NMR (125 MHz, CD₃OD) δ 26.06, 26.45, 27.69, 28.86, 35.30, 36.77, 37.35, 41.66, 55.49, 56.87, 58.16, 68.20, 81.16, 128.11, 129.17, 130.87, 146.11, 157.58, 173.85, 180.79; HRMS-ES (m/z): [M+H] calcd for C₃₆H₄₅N₃O₅S⁺ 634.3314. found 634.3312.

6-(N-methyl-N-(2-(2-amino-3-mercapto-propionamido)ethyl)amino)hexanoic acid, bishydrotrifluoroacetate (9): Et₃SiH (0.036 mL, 0.222 mmol) was added to a solution of sodium 6-(N-methyl-N-(2-(2-tert-butoxycarbonylamino-3-tritylsulfanyl-propionamido)ethyl)amino)hexanoate (8) (72 mg, 0.111 mmol) in TFA (1 mL) and DCM (1 mL) and the mixture was stirred at room temperature under argon for 1 hour. Solvents were removed in vacuo and the residue partitioned between H₂O and Et₂O. The aqueous phase was evaporated in vacuo to yield a white solid. Then, a performic acid reagent solution (a mixture of 30% hydrogen peroxide (5 mL) and 99% formic acid (50 mL) that was allowed to stand at room temperature for 1 hour prior to use, J. Am. Chem. Soc. 1960, 82, 896-903, 18 mL) was added at 0° C. and the solution was stirred for 1 hour. The solvent was removed in vacuo. Water was added and removed in vacuo again. The residue was dried under vacuum to yield a white crystalline solid (0.038 m g, 0.111 mmol, 100%): ¹H NMR (500 MHz, D₂O) δ 1.41 (p, 2H, J=7.5 Hz), 1.65 (p, 2H, J=7.5 Hz), 1.75 (m, 2H), 2.41 (t, 2H, J=7.5 Hz), 2.91 (d, 3H, J=5 Hz), 3.07-3.18 (m, 1H), 3.21-3.34 (m, 2H), 3.38-3.62 (m, 2H), 3.49 (t, 2H, J=6 Hz), 3.78-3.92 (m, 1H), 4.43 (t, 1H, J=6 Hz); ¹³C NMR (125 MHz, D₂O) δ 24.11, 24.72, 26.15, 34.48, 35.62, 35.68, 40.96, 41.03, 50.98, 51.14, 55.81, 57.29, 57.63, 169.37, 179.60; HRMS-ES (m/z): [M] calcd for C₁₂H₂₆N₃O₆S⁺ 340.1542. found 340.1514.

(11): 461.2 mg (1.02 mmol) of 6-(N-methyl-N-(2-(2-amino-3-mercaptopropionamido)ethyl)amino)hexanoic acid, bishydrotrifluoroacetate (9) was combined in a round bottom flask with 295.2 mg (0.852 mmol) of N-benzylcarbonylamino proline O-succinimide and 340 υL (2.44 mmol) of triethylamine in 10 mL of N,N-dimethylformamaide. The reaction was stirred at room temperature for 1.5 h at which time it was complete by TLC. The solvent was removed by lyophilization. The desired product was purified by HPLC Synergi RP-Polar 250×20.2 mm, 4 micron column. 14%-60% 94.9% methanol:5% water 0.1% trifluoroacetic acid:water with 0.1% trifluoroacetic acid over 50 min. 20 mL/min. 254 nm. tᵣₑₜₑₙₜᵢₒₙ=18.8 min. The fractions were lyophilized to yield 442.4 mg (91%) of the desired compound. ¹H-NMR (300 MHz, D₂O) δ7.19-7.26 (m, 5H), 4.86-5.06 (m, 4H), 4.71-4.78 (m, 1H), 4.52 (s, 1H), 4.30 (s, 1H), 4.12-4.18 (m, 2H), 3.33-3.43 (m, 6H), 3.16-3.19 (m, 3H), 3.11 (m, 2H), 3.00 (m, 2H), 2.88 (m, 2H), 2.74 (m, 2H), 2.68 (s, 3H), 2.12-2.22 (m, 4H), 1.75-1.78 (m, 4H), 1.35-1.55 (m, 6H), 1.15-1.25 (m, 3H). Carried forward without further purification.

(12): 591.4 mg (1.04 mmol) of 11 was combined in a round bottom flask with 20 mL of methanol and 220.4 mg (0.21 mmol) of 10% Pd/C. Hydrogen was bubbled through the reaction for 10 minutes then the reaction was stirred under hydrogen atmosphere for 2 h. The reaction was filtered and concentrated to dryness. The residual oil was dissolved in water and washed twice with ethyl acetate (15 mL). The aqueous layer was filtered through a 0.45 micron syringe filter, frozen and lyophilized to provide the product as a white foam (307.3 mg, 68%). ¹H-NMR (500 MHz, D₂O) δ 4.57-4.62 (m, 1H), 4.23-4.26 (m, 1H), 3.55 (bs, 1H), 3.36-3.39 (m, 1H), 3.13-3.28 (m, 7H), 3.09 (bs, 1H), 2.76 (s, 3H), 2.26-2.29 (m, 1H), 2.09-2.11 (t, J=7.5 Hz, 2H), 1.85-1.89 (m, 3H), 1.56 (bs, 2H), 1.44-1.49 (m, 2H), 1.17-1.21 (m, 2H).

### 5-((R)-2-((N-Boc)-amino)-(3-tritylthio)-propionamido)-pentanoic acid (14)

A mixture of 5-aminovaleric acid hydrochloride (13) (1.40 g, 9.11 mmol), Boc-Cys(Trt)-OSu (7) (5.0 g, 8.92 mmol) and Na₂CO₃ (1.89 g, 17.8 mmol) in H₂O (52.5 mL) and 1,4-Dioxane (52.5 mL) was stirred at ambient temperature for 2 d before the solvents were removed in vacuo. The residue was dissolved in H₂O (1 L) and acidified to pH 2 using 1M HCl, at which point a white precipitate fell out of solution. The precipitate was filtered and washed with H₂O, followed by drying under vacuum providing a white solid (4.40 g, 7.82 mmol, 88%): FTIR (CH₂Cl₂): 700 (s), 742 (s), 1166 (s), 1490 (s), 1527 (s), 1708 (s), 2341 (s), 2359 (s), 2931 (s), 2975 (s) 3304 (br); ¹H NMR (500 MHz, d-acetone) 1.44 (s, 9H), 1.52-1.57 (m, 2H), 1.61-1.66 (m, 2H), 2.32 (t, J=7 Hz, 2H), 2.55-2.63 (m, 2H), 3.19-3.26 (m, 2H), 4.10-4.12 (m, 1H), 6.12 (d, J=8 Hz, 1H), 7.27 (t, J=8 Hz, 3H), 7.35 (t, J=7 Hz, 6H), 7.45 (d, J=7.5 Hz, 6H); ¹³C NMR (125 MHz, d-acetone) 22.85, 28.63, 29.66, 33.86, 35.35, 39.56, 54.66, 67.32, 79.66, 127.63, 128.82, 130.39, 145.72, 156.03, 171.01, 174.84; HRMS-ES (m/z): [M+Na] calcd for C₃₂H₃₈N₂O₅SNa⁺ 585.2399. found 585.2393.

### 5-((R)-2-amino-sulfono-propionamido)-pentanoic acid (15)

Triethylsilane (4.58 mL, 28.6 mmol) and TFA (40.4 mL) were added to a solution of 5-((R)-2-((N-Boc)-amino)-(3-tritylthio)-propionamido)-pentanoic acid (14) (8.075 g, 14.3 mmol) in anhydrous DCM (40.4 mL) and the mixture was stirred at ambient temperature for 1.25 h before the solvents were removed in vacuo. The white residue was partitioned between Et₂O and H₂O. The aqueous phase was removed in vacuo providing a yellow oil which was dissolved a performic acid reagent solution (a mixture of 30% hydrogen peroxide (5 mL) and 99% formic acid (50 mL) that was allowed to stand at room temperature for 1 hour prior to use) and stirred at ambient temperature for 1.5 h. The solvent was removed in vacuo, and the white solid was dissolved in water. The water was removed in vacuo. The white crystalline product was dried under vacuum providing a white solid (3.384 g, 11.6 mmol, 81%): ¹H NMR (500 MHz, d-DMSO) δ 1.43 (m, 2H), 1.50 (m, 2H), 2.21 (t, J=7 Hz, 2H), 2.76 (dd, J=13.5, 10.5 Hz, 1H), 2.96 (dd, J=14, 2 Hz), 3.09 (m, 2H), 3.98 (d, J=8 Hz, 1H), 8.62 (d, J=4.5 Hz, 1H) (NH); ¹³C NMR (125 MHz, d-DMSO) δ 21.77, 28.12, 33.20, 38.55, 50.24, 50.48, 166.95, 174.30; HRMS-ES (m/z): [M+Na] calcd for C₈H₁₆N₂O₆SNa⁺ 291.0627. found 291.0609.

5-((R)-2-(benzyl(R)-2-carbamoylpyrrolidine-1-carboxyloyl)-2-carbamoylethane-sulfonato) pentanoic acid (16): A mixture of Cbz-(D)-Pro-OSu (10) (165 mg, 0.476 mmol), 5-((R)-2-amino-sulfono-propionamido)-pentanoic acid (15) (128 mg, 0.477 mmol) and Et₃N (2.00 mL, 14.4 mmol) in anhydrous DMF (25 mL) was stirred at ambient temperature for 1 d. The solvent was removed via the use of a lyophilizer, and the crude product was purified via reverse phase HPLC using a gradient of 1:19 ([95% CH₃CN/4.9% H₂O/0.1% TFA]:[99.9% H₂O/0.1% TFA]) to 1:4 ([95% CH₃CN/4.9% H₂O/0.1% TFA]:[99.9% H₂O/0.1% TFA]) over 30 min at a flow rate of 20 mL/min, monitoring at 220 nm. The product was collected at 20 min. The solution containing the product was frozen, and the solvents removed via the use of a lyophilizer providing white crystals (166 mg, 0.333 mmol, 70%): ¹³C NMR (125 MHz, CD₃OD) δ 23.34, 25.80, 26.92, 27.01, 29.63, 31.01, 33.83, 34.46, 40.50, 48.31, 50.38, 52.23, 52.56, 62.50, 68.38, 128.77, 129.16, 129.67, 138.31, 157.01, 172.32, 175.27, 175.77; HRMS-ES (m/z): [M] calcd for C₂₁H₂₈N₃O₉S⁻ 498.1552. found 498.1540.

### 5-(2-carbamoyl-(R)-2-(R)-pyrrolidine-2-carboxamido)ethanesulfonyl)pentanoic acid (17)

A suspension of 5-((R)-2-(benzyl(R)-2-carbamoylpyrrolidine-1-carboxyloyl)-2-carbamoylethane-sulfonato) pentanoic acid (16) (160 mg, 0.321 mmol) and 10% Pd/C (200 mg, 0.188 mmol) in EtOH (15 mL) was evacuated and charged with hydrogen gas several times before allowing the mixture to stir under hydrogen (1 atm) at ambient temperature for 3 h. The mixture was filtered, and the bluish solid which remained was rinsed with a 1:1 MeOH/H₂O mixture to dissolve the product. Removal of the solvents in vacuo provided a white solid (115 mg, 0.315 mmol, 100%): ¹H NMR (500 MHz, d-DMSO) δ 1.48 (m, 2H), 1.53 (m, 2H), 2.01 (p, J=7 Hz, 2H), 2.14 (m, 1H), 2.05 (t, J=7 Hz, 2H), 2.38 (m, 1H), 3.12-3.19 (m, 3H), 3.24-3.28 (m, 2H) 3.30-3.34 (m, 1H), 3.37-3.42 (m, 1H), 4.36 (t, J=7 Hz, 1H), 4.71 (dd, J=9.5, 3 Hz, 1H), 8.21 (t, J=5.5 Hz, 1H) (NH); ¹³C NMR (125 MHz, d-DMSO) δ 21.83, 23.37, 28.49, 28.96, 33.29, 38.40, 39.85, 51.24, 52.00, 59.22, 59.71, 167.69, 169.83, 174.41; HRMS-ES (m/z): [M+Na] calcd for C₁₃H₂₃N₃O₇SNa⁺ 388.1149. found 388.1155.

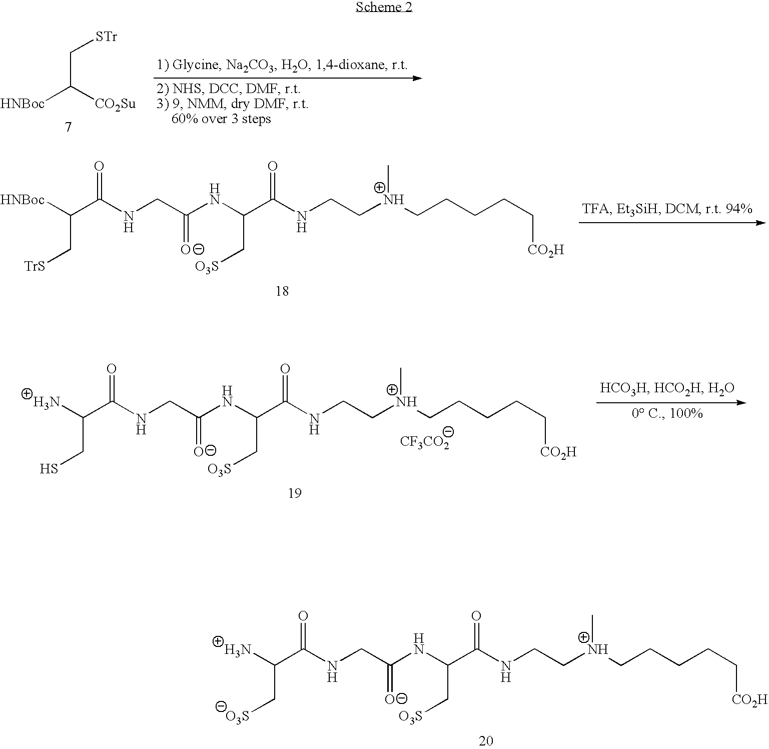

### N—(N-methyl-N-(2-(2-(2-(2-tert-butoxycarbonylamino-3-tritylsulfanyl-propionamido)acetamido)-3-sulfonate-propionamido)ethyl)amino)hexanoic acid (18)

A solution of Boc-Cys(Trt)-OSu (7) (1 g, 1.78 mmol), glycine (200 mg, 2.67 mmol) and Na₂CO₃ (566 mg, 5.34 mmol) in 1,4-dioxane (5 mL): water (8 mL) was stirred at room temperature for 19 hours. The solution was brought to pH 5 with 1M HCl, and the resulting mixture partitioned between water and EtOAc. The combined organic extracts were dried over Na₂SO₄ and evaporated in vacuo. To the crude product was added N-hydroxysuccinimide (409 mg, 3.56 mmol) and DCC (367 mg, 1.78 mmol). Anhydrous DMF (10 mL) was added and the resulting solution was stirred 3 hours at room temperature under argon. The resulting DCU was filtered out and the filtrate was evaporated in vacuo. The resulting solid was dissolved in EtOAc and washed with water (×3). The EtOAc extract was dried over Na₂SO₄ and evaporated in vacuo. The crude mixture was partially resolved by DAVISIL flash chromatography (60% EtOAC:40% hexanes) to yield unclean product (1.10 g). 6-(N-methyl-N-(2-(2-amino-3-sulfonate-propionamido)ethyl)amino)hexanoic acid, hydroformate (9) (62 mg, 0.162 mmol) was added to a fraction of this crude material (100 mg) in anhydrous DMF (1 mL). Triethylamine (0.135 mL, 0.972 mmol) was added to the solution and the mixture was stirred at room temperature for 19 hours. The solvent was removed in vacuo at room temperature and the resulting mixture purified by reverse phase HPLC to yield a white solid. (82 mg, 0.0972 mmol, 60%): ¹H NMR (600 MHz, d6-acetone) δ 1.30-1.47 (m, 2H), 1.42 (s, 9H), 1.62 (m, 2H), 1.76 (m, 2H), 2.32 (q, 2H, J=8.1 Hz), 2.60-4.30 (m, 16H), 4.55-4.72 (m, 1H), 6.30 (m, 1H), 7.25 (t, 3H, J=7.2 Hz), 7.33 (t, 6H, J=7.2 Hz), 7.40 (d, 6H, J=7.2 Hz) 7.93-8.72 (m, 3H), 9.44 (br, 2H); ¹³C NMR (150 MHz, d6-acetone) δ 24.26, 25.17, 26.88, 29.01, 34.32, 35.37, 41.06, 41.24, 44.63, 51.94, 52.60, 56.91, 57.16, 57.27, 57.79, 67.90, 80.48, 127.91, 129.09, 130.76, 146.13, 156.40, 170.00, 172.74, 173.06, 173.19, 175.05; HRMS-ES (m/z): [M+H] calcd for C₄₁H₅₅N₅O₁₀S₂ 842.3463. found 842.3498.

6-(N-methyl-N-(2-(2-(2-(2-amino-3-mercapto-propionamido)acetamido)-3-sulfonate-propionamido)ethyl)amino)hexanoic acid, hydrotrifluoroacetate (19): To a solution of 6-(N-methyl-N-(2-(2-(2-(2-tert-butoxycarbonylamino-3-tritylsulfanyl-propionamido)acetamido)-3-sulfonate-propionamido)ethyl)amino)hexanoic acid (18) (70 mg, 0.0831 mmol) in anhydrous DCM (1 mL) and TFA (1 mL) was added triethyl silane (27 μL, 0.166 mmol). The mixture was stirred at room temperature under argon for 1 hour. The solvents were removed in vacuo. The resulting oil was partitioned between water and diethyl ether. The aqueous phase was evaporated in vacuo to yield a white solid (48 mg, 0.0781 mmol, 94%): ¹H NMR (500 MHz, D₂O) δ 1.35 (p, 2H, J=7.5 Hz), 1.60 (p, 2H, J=7.5 Hz), 1.70 (m, 2H), 2.37 (t, 2H, J=7.5 Hz), 2.85 (s, 3H), 3.00-3.13 (m, 3H), 3.16-3.42 (m, 5H), 3.50-3.70 (m, 2H), 4.02 (s, 2H), 4.26 (t, 1H, J=5.5 Hz), 4.69 (t, 1H, J=6.0 Hz); ¹³C NMR (150 MHz, D₂O) δ 23.13, 23.68, 24.81, 25.11, 33.43, 34.57, 34.60, 40.11, 42.68, 50.64, 50.77, 50.91, 54.51, 55.10, 56.36, 56.41, 168.77, 170.81, 172.14, 178.62; [M] calcd for C₁₇H₃₄N₅O₈S₂⁺ 500.1843. found 500.1849.

6-(N-methyl-N-(2-(2-(2-(2-amino-3-sulfonate-propionamido)acetamido)-3-sulfonate-propionamido)ethyl)amino)hexanoic acid (20): A mixture of 30% hydrogen peroxide (0.2 mL) and 99% formic acid (1.8 mL) was allowed to stand at room temperature for 1 hour prior to use (J. Am. Chem. Soc. 1960, 82, 896-903). The performic acid reagent solution (2 mL) was cooled to 0° C. and added at the same temperature to a flask containing 6-(N-methyl-N-(2-(2-(2-(2-amino-3-mercapto-propionamido)acetamido)-3-sulfonate-propionamido)ethyl)amino)hexanoic acid, hydrotrifluoroacetate (19) (48 mg, 0.0784 mmol). The mixture was stirred at room temperature for 10 min. The solvent was removed in vacuo. Water was added and removed in vacuo again. The residue was dried under vacuum to yield a white crystalline solid (43 mg, 0.0784 mmol, 100%): ¹H NMR (600 MHz, D₂O) δ 1.37 (br, 2H), 1.62 (br, 2H), 1.71 (m, 2H), 2.38 (br, 2H), 2.87 (s, 3H), 3.08 (br, 1H), 3.24 (br, 2H), 3.30-3.55 (m, 5H), 3.61 (br, 2H), 4.03 (s, 2H), 4.49 (s, 1H), 4.72 (s, 1H); ¹³C NMR (150 MHz, D₂O) δ 24.42, 25.00, 26.44, 34.76, 35.95, 41.49, 44.32, 51.36, 51.40, 52.05, 52.10, 56.43, 57.75, 169.72, 172.28, 173.52, 179.88; HRMS-ES (m/z): [M+H] calcd for C₁₇H₃₃N₅O₁₁S₂ 548.1691. found 548.1687.

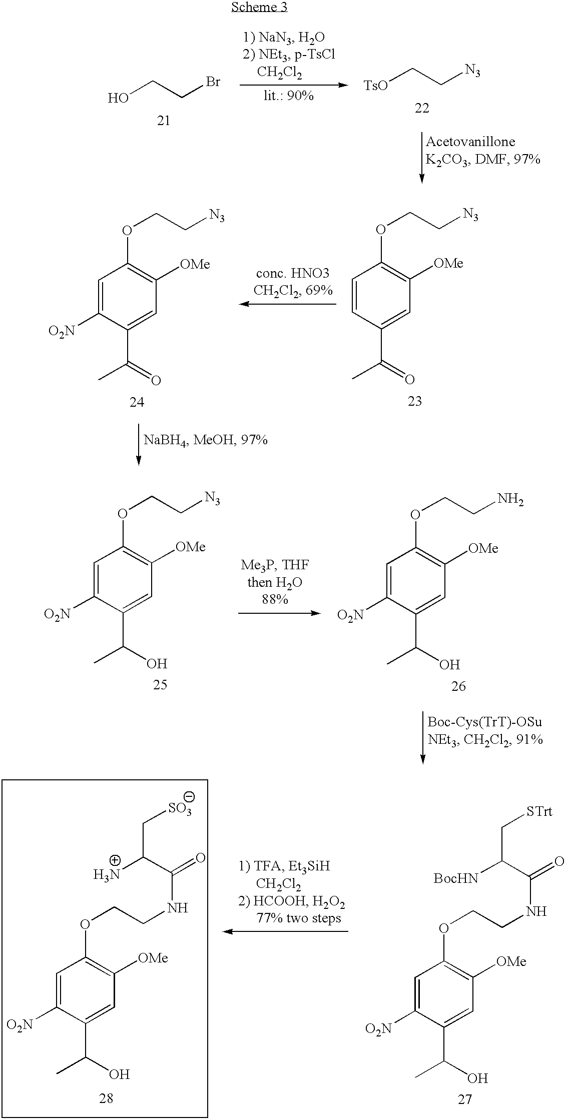

(23): Toluenesulfonic acid-2-azido ethyl ester (22) (1.67 g, 6.94 mmol, 1.09 eq.) which was prepared according to Demko et al., Org. Lett. 2001, 3, 4091-4094 was added to a solution of acetovanillone (1.06 g, 6.37 mmol, 1 eq.) and freshly grinded potassium carbonate (1.3 g, 9.36 mmol, 1.47 mmol) in 20 ml dry DMF (3.1 ml/mmol). This suspension was stirred overnight at 80-100° C. The solvent was evaporated under reduced pressure and the residue was purified by flash chromatography through a short column of silica gel (10/1 to 1/1 hexane/EtOAc) to give 22 (1.46 g, 6.19 mmol, 97%). ¹H NMR (300 MHz, CDCl₃) δ 2.55 (s, 3H), 3.66 (t, J=5.1 Hz, 2H), 3.90 (s, 3H), 4.22 (t, J=5.1 Hz, 2H), 6.88 (d, J=9 Hz, 1H), 7.50-7.58 (m, 2H).

(24): Fuming nitric acid (1.85 ml, 0.3 ml/mmol) was slowly added to an ice-cooled solution of 23 (1.45 g, 6.17 mmol) in 18.5 ml CH₂Cl₂ (3 ml/mmol). Then the ice bath was removed and the dark red solution stirred overnight. The reaction mixture was quenched with water, followed by extraction with CH₂Cl₂. Drying of the organic phase with MgSO₄ and evaporation of the solvent gave a brownish residue which was purified by flash chromatography through a short column of silica gel (10/1 to 1/1 hexane/EtOAc) to give 24 (1.187 g, 4.23 mmol, 69%). ¹H NMR (300 MHz, CDCl₃) δ 2.48 (s, 3H), 3.69 (t, J=5.1 Hz, 2H), 3.95 (s, 3H), 4.25 (t, J=5.1 Hz, 2H), 6.75 (s, 1H), 7.62 (s, 1H).

(25): Sodium borohydride (192 mg, 5.1 mmol, 1.2 eq.) was added to a solution of 24 (1.187 g, 4.23 mmol) in 10 ml MeOH (2.4 ml/mmol) at 0° C. The ice bath was then removed and the mixture stirred for 30 min at room temperature. The reaction mixture was quenched with saturated aqueous NH₄Cl, followed by extraction with CH₂Cl₂. Drying of the organic phase with MgSO₄ and evaporation of the solvent gave a yellow oil which was purified by flash chromatography through a short column of silica gel (10/1 to 1/1 hexane/EtOAc) to give 25 (1.157 g, 4.10 mmol, 97%). ¹H NMR (300 MHz, CDCl₃) δ 1.42 (d, J=6.5 Hz, 3H), 3.05 (br. s, 1H), 3.58 (t, J=5.1 Hz, 2H), 3.89 (s, 3H), 4.15 (t, J=5.1 Hz, 2H), 5.45 (q, J=7.0 Hz, 1H), 7.27 (s, 1H), 7.47 (s, 1H).

(26): To a solution of 25 (453 mg, 1.62 mmol) in 8 ml THF (5 ml/mmol) was added at 0° C. a 1 M solution of trimethylphosphine in toluene (2 ml, 1.94 mmol, 1.2 eq.). After 2.5 h the reaction was quenched with water and stirred for 12 h at room temperature. The mixture was extracted with CH₂Cl₂. Drying of the organic phase with MgSO₄ and evaporation of the solvent gave a yellow oil which was purified by flash chromatography through a short column of silica gel (10/1 to 1/1 hexane/EtOAc containing 1% NEt₃) to give 26 (364 mg, 1.42 mmol, 88%). ¹H NMR (300 MHz, CDCl₃) δ 1.44 (d, J=6.3 Hz, 3H), 3.05 (t, J=5.2 Hz, 2H), 3.89 (s, 3H), 3.98 (t, J=5.1 Hz, 2H), 5.47 (q, J=6.2 Hz, 1H), 7.28 (s, 1H), 7.45 (s, 1H).

(27): To a solution of the amine 26 (150 mg, 0.59 mmol) in 10 ml CH₂Cl₂ (17 ml/mmol) was added Boc-Cys(Trt)-OSu (7) (347 mg, 0.62 mmol, 1.05 eq.) and triethylamine (119 mg, 1.18 mmol, 0.17 ml, 2 eq.). The solution was stirred for 90 min at room temperature followed by evaporating the solvent under reduced pressure. The residue was purified by flash chromatography through a short column of silica gel (5/1 to 1/1 hexane/EtOAc) to give 27 (377 mg, 0.54 mmol, 91%). ¹H NMR (300 MHz, CDCl₃) δ 1.37 (s, 9H), 1.54 (d, J=6.3 Hz, 3H), 2.43-2.53 (m^{AB}, 1H), 2.65-2.75 (m^{AB}, 1H), 3.60-3.68 (m, 2H), 3.92 (s, 3H), 4.03-4.10 (m, 2H), 4.78 (br. m, 1H), 5.50-5.60 (m, 1H), 7.15-7.55 (m, 17H).

(28): To a solution of 27 (366 mg, 0.52 mmol) in 5 ml CH₂Cl₂ (10 ml/mmol) was added triethylsilane (121 mg, 1.04 mmol, 0.17 ml, 2 eq.) and 1 ml trifluoroacetic acid (2 ml/mmol) at 0° C. The ice bath was removed and the solution stirred for 90 min at room temperature. The solvent was removed under reduced pressure. The resulting paste was partitioned between water and ether. The aqueous phase was evaporated to give crude thiol which was dissolved in 9 mL of a performic acid reagent solution (a mixture of 30% hydrogen peroxide (5 mL) and 99% formic acid (50 mL) that was allowed to stand at room temperature for 1 hour prior to use) and stirred at room temperature for 1.5 hour. The solvent was removed under reduced pressure to yield 28 (164 mg, 0.40 mmol, 77%) as a yellow-orange crystalline solid. ¹H NMR (500 MHz, D₂O) δ 1.22 (d, J=6.3 Hz, 3H), 3.05-3.20 (m, 2H), 3.35-3.45 (m^{AB}, 1H), 3.49-3.56 (m^{AB}, 1H), 3.74 (s, 3H), 3.93-4.01 (m, 2H), 4.15-4.20 (m, 1H), 5.21 (q, J=6.3 Hz, 1H), 7.05 (s, 1H), 7.37 (s, 1H).

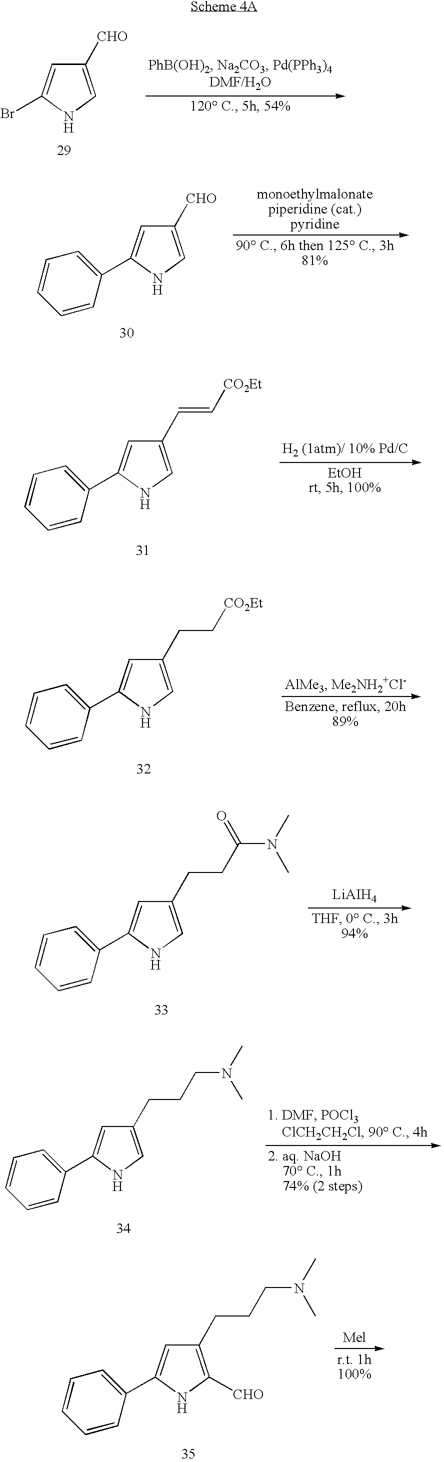

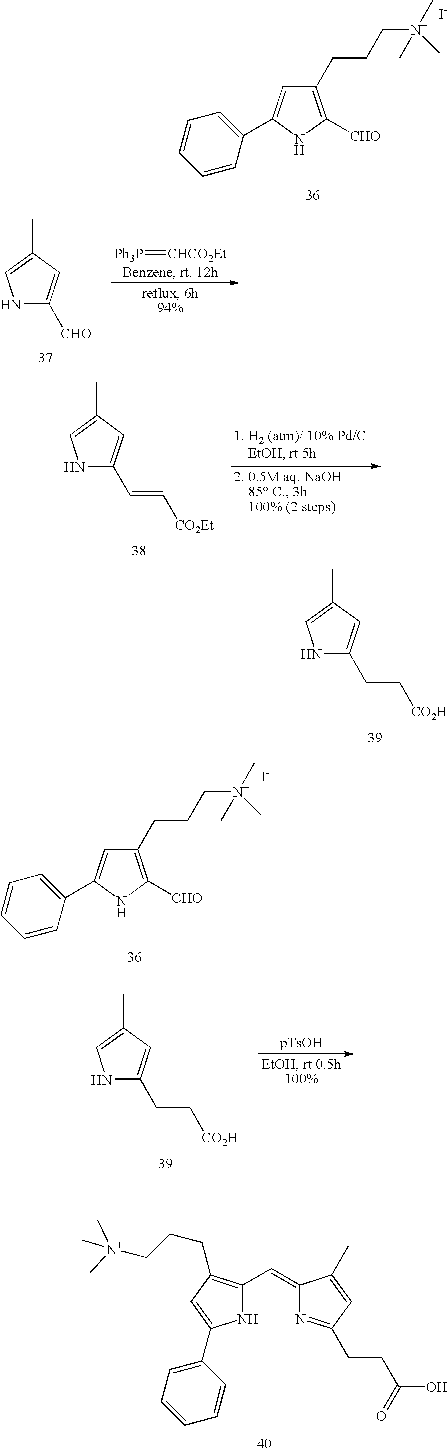

2-bromo-4-formylpyrrole (29): To a cold (−78° C.) solution of 3-formylpyrrole (9.1 g; 95.8 mmol) in anhydrous THF (400 mL) was added freshly recrystallized N-bromosuccinimide (17 g; 95.8 mmol). The reaction mixture was brought to −20° C. and stirred under argon for 15 hours. The solvent was removed in vacuo and the crude residue was purified by flash chromatography on silica gel (10% ethylacetate; 90% hexanes). The fractions containing the product were further purified on a second column (2% iPrOH; 98% hexanes) to yield a white powder (10.7 g, 61.3 mmol, 64%): mp 119-121° C. (dec); FTIR (CH₂Cl₂) 1643 (CO); ¹H NMR (500 MHz, acetone-d₆ δ) 6.58 (d, 1H, J=1.6 Hz), 7.67 (d, 1H, J=1.6 Hz), 9.71 (s, 1H); ¹³C NMR (125 MHz, acetone-d₆) δ 102.83, 109.10, 129.01, 129.90, 184.75; TLC R_{f}=0.16 (20% EtOAc in hexanes). HRMS-EI (m/z): [M] calcd for C₅H₄BrNO 172.9476. found 172.9481.

2-phenyl-4-formylpyrrole (30): To a solution of 2-bromo-4-formylpyrrole (29) (7.5 g, 43.1 mmol) and palladium tetrakis-triphenylphosphine (1 g, 0.864 mmol) in degassed DMF (170 mL) was added under argon via syringe a solution of Na₂CO₃ (11.2 g, 105 mmol) in degassed water (70 mL). The mixture was stirred at room temperature for 5 min and a solution of phenylboronic acid (6 g, 49.4 mmol) in degassed DMF (80 mL) was added. The reaction mixture was then stirred under argon at 120° C. for 5 hours. The flask was allowed to cool down to room temperature and water was added. The resulting solution was extracted with CH₂Cl₂. The organic phase was washed with water (×5), brine (×3) and dried over Mg₂SO₄. The solvent war removed in vacuo. The resulting solid was purified by flash chromatography on silica gel (10% ethylacetate; 90% hexanes) to yield a white powder: (4 g, 23.4 mmol, 54%): mp 136-137° C.; FTIR (CH₂Cl₂) 1642 (CO); ¹H NMR (300 MHz, acetone-d₆) δ 6.96 (s, 1H), 7.26 (t, 1H, J=7.4 Hz), 7.40 (t, 2H, J=7.4 Hz), 7.70 (s, 1H), 7.72 (d, 2H, J=7.4 Hz); ¹³C NMR (125 MHz, acetone-d₆) δ 104.17, 125.18, 127.92, 129.11, 129.81, 132.77, 135.50, 185.70; TLC R_{f}=0.06 (20% EtOAc in hexanes). HRMS-EI (m/z): [M] calcd for C₁₁H₉NO 171.0684. found 171.0680.

(E)-ethyl-3-(5-phenyl-1H-3-pyrrolyl)acrylate (31): To a stirred solution of 2-phenyl-4-formylpyrrole (30) (800 mg, 4.68 mmol) and piperidine (92 μL, 0.936 mmol) in anhydrous pyridine (4.28 mL, 52.9 mmol) was added monoethyl malonate (3.31 mL, 28.1 mmol). The reaction mixture was heated under argon to 90° C. for 6 hours. Heating was raised to 125° C. and stirring was continued for an additional 3 hours. The reaction flask was allowed to cool down to room temperature and water was added (130 mL). The solution was brought to pH 1 by addition of 1M HCl and extracted exhaustively with EtOAc. The organic extract was dried over anhydrous K₂CO₃. The solvent was removed in vacuo and the resulting oily residue was triturated in hexanes to produce a yellowish solid (911 mg, 3.78 mmol, 81%). An analytical sample was obtained by recrystallization from CH₂Cl₂ and hexanes to yield white crystals: mp 67-70° C.; FTIR (CH₂Cl₂) 1276 (s), 1625 (s), 1672 (s), 2359 (d), 3307 (s); ¹H NMR (300 MHz, acetone-d₆) δ 1.27 (3, 3H, J=7.1), 4.17 (q, 2H, J=7.1 Hz), 6.18 (d, 1H, J=15.7 Hz), 6.94 (s, 1H), 7.23 (t, 1H, J=7.4 Hz), 7.30 (s, 1H), 7.39 (t, 2H, J=7.4 Hz), 7.62 (d, 1H, J=15.7 Hz), 7.69 (d, 2H, J=7.4 Hz); ¹³C NMR (125 MHz, acetone-d₆) δ 14.83, 60.20, 104.35, 113.79, 122.87, 124.48, 124.91, 127.47, 129.79, 133.39, 139.82, 168.02; TLC R_{f}=0.44 (20% EtOAc in hexanes). HRMS-EI (m/z): [M] calcd for C₁₅H₁₅NO₂ 241.1103. found 241.1102.

Ethyl-3-(5-phenyl-1H-3-pyrrolyl)propanoate (32): (E)-ethyl 3-(5-phenyl-3-pyrrolyl)acrylate (31) (860 mg, 3.57 mmol) was dissolved in absolute ethanol (30 mL). 10% Pd on carbon (120 mg, 0.107 mmol) was added to the solution and the resulting suspension was stirred under hydrogen (1 atm) for 5 hours. The catalyst was filtered out and rinsed with ethanol. The filtrate was evaporated in vacuo to yield a white powder (866 mg, 100%): mp 141-143° C.; FTIR (CH₂Cl₂) 1718 (s), 2359 (s), 3350 (s); ¹H NMR (300 MHz, acetone-d₆) δ 1.21 (t, 3H, J=7.1 Hz), 2.55 (t, 2H, J=7.6 Hz), 2.77 (t, 2H, J=7.6 Hz), 4.09 (q, 2H, J=7.1 Hz), 6.42 (s, 1H), 6.68 (s, 1H), 7.13 (t, 1H, J=7.4 Hz), 7.25 (t, 2H, J=7.4 Hz), 7.58 (d, 2H, J=7.4 Hz); ¹³C NMR (125 MHz, acetone-d₆) δ 14.66, 23.39, 36.62, 60.53, 106.76, 117.42, 124.67, 126.36, 134.41, 173.49; TLC R_{f}=0.21 (20% EtOAc in hexanes). HRMS-EI (m/z): [M] calcd for C₁₅H₁₇NO₂ 243.1259. found 243.1265.

N,N-dimethyl-3-(5-phenyl-1H-3-pyrrolyl)propanamide (33): A solution of trimethylaluminum (2M, 12.2 mL, 24.4 mmol) in toluene was added dropwise to a suspension of dimethylammonium chloride (1.99 g, 24.4 mmol) in anhydrous benzene (100 mL). The reaction was stirred at room temperature under argon for 1 hour. A solution of ethyl 3-(5-phenyl-1H-3-pyrrolyl)propanoate (32) (2.96 g, 12.2 mmol) in anhydrous benzene (100 mL) was added drop-wise to the reaction mixture. The reaction was refluxed for 20 hours. The mixture was allowed to cool down to room temperature and aqueous HCl was added (2M, 72 mL). The reaction flask was placed in an ice bath. The resulting precipitate was collected by filtration to yield an off white solid (2.4 g). The liquor was extracted with ethyl acetate. The organic layer was dried over Mg₂SO₄ and evaporated under vacuum. The resulting solid was recrystallized from hexane to yield an additional 200 mg of off white solid (combined yield: 2.6 g, 10.7 mmol, 89%): mp 183-184° C.; FTIR (CH₂Cl₂) 1623 (s), 2420 (s), 3250 (s); ¹H NMR (300 MHz, CD₃OD) δ 2.64 (t, 2H, J=6.7), 2.78 (t, 2H, J=6.7 Hz), 2.93 (s, 3H), 3.00 (s, 3H), 6.36 (s, 1H), 6.62 (s, 1H), 7.11 (t, 1H, J=7.4 Hz), 7.29 (t, 2H, J=7.4 Hz), 7.51 (d, 2H, J=7.4 Hz); ¹³C NMR (125 MHz, CD₃OD) δ 24.27, 35.94, 36.29, 38.08, 106.64, 117.78, 124.58, 124.98, 126.58, 129.81, 133.30, 134.98; TLC R_{f}=0.37 (EtOAc). HRMS-EI (m/z): [M] calcd for C₁₅H₁₈N₂O 242.1419. found 242.1417.

N,N-dimethyl-3-(5-phenyl-1H-3-pyrrolyl)propan-1-amine (34): To a suspension of lithium aluminum hydride (454 mg, 11.9 mmol) in anhydrous THF (40 mL) maintained at 0° C. was added a solution of N,N-dimethyl-3-(5-phenyl-1H-3-pyrrolyl)propanamide (33) (567 mg, 2.34 mmol) in anhydrous THF (75 mL). The mixture was then stirred under argon at room temperature for 3 hours. The reaction was quenched by addition of aqueous Na₂CO₃ (1 M, 200 mL) and the mixture was extracted with EtOAc. The organic extract was dried over Na₂SO₄ and the solvent was removed in vacuo to yield a solid (500 mg, 2.2 mmol, 94%): mp 79-80° C.; FTIR (CH₂Cl₂) 763 (s), 1465 (s), 1513 (s), 1606 (s), 2939 (s); ¹H NMR (500 MHz, CD₃OD) δ 1.73 (m, 2H), 2.18 (s, 6H), 2.31 (m, 2H), 2.44 (t, 2H, J=7.4 Hz), 6.31 (d, 1H, J=1.5 Hz), 6.56 (d, 1H, J=1.5 Hz), 7.07 (t, 1H, J=7.4 Hz), 7.26 (t, 2H, J=7.4 Hz), 7.49 (d, 2H, J=7.4 Hz); ¹³C NMR (75 MHz, CD₃OD) δ 26.04, 29.92, 45.53, 60.53, 106.67, 117.65, 124.54, 125.73, 126.48, 129.79, 133.13, 135.02; TLC R_{f}=0.06 (20% MeOH in CH₂Cl₂). HRMS-EI (m/z): [M] calcd for C₁₅H₂₀N₂ 228.1626. found 228.1628.

3-(3-(dimethylamino)-propyl)-5-phenyl-1H-pyrrole-2-carboxaldehyde (35): Anhydrous DMF (0.677 mL, 8.76 mmol) was added under argon to a flask containing POCl₃ (0.4 mL, 4.38 mmol). The solution was stirred at room temperature for 1 hour. (CH₂)₂Cl₂ (20 mL) and a solution of N,N-dimethyl-3-(5-phenyl-1H-3-pyrrolyl)propan-1-amine (34) (105 mg, 0.438 mmol) in (CH₂)₂Cl₂ (24 mL) were successively added to the reaction mixture. The resulting solution was refluxed under argon at 90° C. for 4 hours. The reaction mixture was poured on crushed ice and brought to pH 12 by addition of aqueous NaOH (10 M). The mixture was heated to 70° C. for 1 hour and allowed to cool down to room temperature. The crude mixture was extracted with EtOAc and the organic phase was dried over Mg₂SO₄. The solvent war removed in vacuo. The resulting solid was purified by flash chromatography on silica gel (40% MeOH; 60% CH₂Cl₂) to yield a light brown solid (1.17 g, 0.325 mmol, 74%): mp 77-79° C.; FTIR (CH₂Cl₂) 1469 (d), 1632 (s), 2777 (s), 2815 (s), 2942 (s), 3272 (br); ¹H NMR (500 MHz, CD₃OD) δ 1.87 (m, 2H), 2.40 (m, 2H), 2.84 (t, 2H, J=7.5 Hz), 6.60 (s, 1H), 7.34 (t, 1H, 7.4 Hz), 7.42 (t, 2H, J=7.4 Hz), 7.74 (d, 2H, J=7.4 Hz), 9.59 (s, 1H); ¹³C NMR (125 MHz, CD₃OD) δ 24.54, 30.03, 45.58, 60.26, 110.64, 126.72, 129.54, 130.15, 131.38, 132.49, 140.05, 141.58, 179.21; TLC R_{f}=0.19 (50% MeOH; 50% CH₂Cl₂); HRMS-EI (m/z): [M] calcd for C₁₆H₂₀N₂O 256.1576. found 256.1567.

Trimethyl-[3-(2-formyl-5-phenyl-1H-3-pyrrolyl)-propyl]-ammonium iodide (36): To a round bottom flask containing 3-(3-(dimethylamino)-propyl)-5-phenyl-1H-pyrrole-2-carboxaldehyde (35) (40 mg, 0.156 mmol) under argon was added methyl iodide (1 mL). The mixture was stirred at room temperature for one hour. The excess methyl iodide was removed in vacuo to yield an off white powder (42 mg, 0.156 mmol, 100%): mp 230-232° C.; ¹H NMR (500 MHz, CD₃OD) δ 2.20 (m, 2H), 2.95 (t, 2H, J=7.5 Hz), 3.14 (s, 9H), 3.43 (m, 2H), 6.69 (s, 1H), 7.35 (t, 1H, J=7.4 Hz), 7.44 (t, 2H, J=7.4 Hz), 7.75 (d, 2H, J=7.4 Hz), 9.65 (s, 1H); ¹³C NMR (125 MHz, CD₃OD) δ 21.98, 23.57, 52.27, 65.01, 109.35, 125.31, 127.98, 128.77, 129.83, 130.66, 138.38, 177.91; HRMS-EI (m/z): [M] calcd for C₁₇H₂₃N₂O⁺ 271.1805. found 271.1806.

E-Ethyl 3-(4-methyl-1H-2-pyrrolyl)-propanoate (38): A solution of 2-formyl-4-methyl pyrrole (37) (2.6 g, 23.9 mmol) and (carbethoxymethylene)-triphenylphosphorane (12.4 g, 35.7 mmol) in anhydrous benzene (250 mL) was stirred at room temperature under argon overnight. The mixture was then refluxed for 6 hours. Benzene was removed in vacuo and the crude mixture was purified by silica gel flash chromatography (20% EtOAC; 80% hexanes) to yield a white powder (2.17 g, 22.5 mmol, 94%): mp 65° C.; FTIR (CH₂Cl₂) 1603.1 (s), 703.3 (s), 813.8 (s), 969.8 (s), 1184 (s), 1277 (d), 1442 (s), 1571 (s), 1614 (s), 1682 (s), 2969 (br), 3330 (br); ¹H NMR (300 MHz, CD₃OD) δ 1.27 (t, 3H, 7.1 Hz), 2.04 (s, 3H), 4.15 (q, 2H, J=7.1 Hz), 6.01 (d, 1H, J=15.8 Hz), 6.31 (s, 1H), 6.68 (s, 1H), 7.43 (d, 1H, J=15.8 Hz); ¹³C NMR (75 MHz, CD₃OD) δ 11.87, 14.81, 61.27, 110.32, 116.95, 122.05, 122.82, 129.59, 136.54, 170.19; TLC R₁=0.44 (20% EtOAc; 80% hexanes); HRMS-EI (m/z): [M] calcd for C₁₀H₁₃NO₂ 179.0946. found 179.0944.

3-(4-methyl-1H-2-pyrrolyl)-propanoic acid (39): E-Ethyl 3-(4-methyl-1H-2-pyrrolyl)propanoate (38) (225 mg, 1.26 mmol) was dissolved in absolute ethanol (10 mL). 10% Pd on carbon (34 mg, 0.0321 mmol) was added to the solution and the resulting suspension was stirred under hydrogen (1 atm) for 5 hours. The catalyst was filtered out and rinsed with ethanol. The filtrate was evaporated in vacuo to yield a yellow oil. Then, a solution of aqueous NaOH (0.5 M, 30 mL) was added and the mixture was stirred at 85° C. for 3 hours. The mixture was cooled down by addition of iced water and acidified to pH 1 with aqueous HCl (6M). The resulting solution was extracted with EtOAc. The combined organic extracts were dried over Mg₂SO₄ and evaporated in vacuo to yield a brown solid (192 mg, 100%): mp 109-112° C.; ¹H NMR (500 MHz, CD₃OD) δ 2.00 (s, 3H), 2.55 (t, 2H, J=15 Hz), 2.79 (t, 2H, J=15 Hz), 5.64 (s, 1H, 15.8 Hz), 6.32 (s, 1H); ¹³C NMR (125 MHz, CD₃OD) δ 12.17, 24.25, 33.00, 106.60, 107.28, 118.79, 131.97, 177.18; HRMS-EI (m/z): [M] calcd for C₈H₁₁NO₂ 154.0868. found 154.0878.

5-phenyl-3-(3-trimethylammonium iodide)-propyl-3′-methyl-5′-(3-propionic acid) dipyrromethene (40): p-TsOH monohydrate (48 mg, 0.251 mmol), was added to a stirred solution of trimethyl-[3-(2-formyl-4-phenyl-1H-3-pyrrolyl)-ethyl]-ammonium iodide (36) (100 mg, 0.251 mmol) and 3-(4-methyl-1H-2-pyrrolyl)-propanoic acid (39) (42 mg, 0.276 mmol) in absolute ethanol (4 mL). The mixture was stirred 30 min at room temperature. The reaction mixture was then passed through a DOWEX 21K Cl anion exchange resin and eluted with water. When the wash from the column came out clean elution was stopped. The eluate was evaporated in vacuo to yield a red solid (110 mg, 0.251 mmol, 100%): ¹H NMR (500 MHz, CD₃OD) δ 2.27 (m, 2H), 2.47 (s, 3H), 2.82 (t, 2H, 7 Hz), 2.98 (t, 2H, 7.5 Hz), 3.14 (t, 2H, 7.5 Hz), 3.19 (s, 9H), 3.51 (m, 2H), 6.42 (s, 1H), 7.08 (s, 1H), 7.4-7.49 (m, 4H), 7.88-7.95 (m, 2H)); ¹³C NMR (125 MHz, CD₃OD) δ 12.42, 23.99, 24.85, 25.18, 33.00, 53.85, 67.29, 115.95, 118.92, 123.27, 128.64, 130.08, 130.25, 130.56, 131.02, 132.51, 150.14, 150.80, 154.31, 161.84, 175.47; HRMS-ES (m/z): [M] calcd for C₂₅H₃₂N₃O₂⁺ 406.2495. found 406.2477.

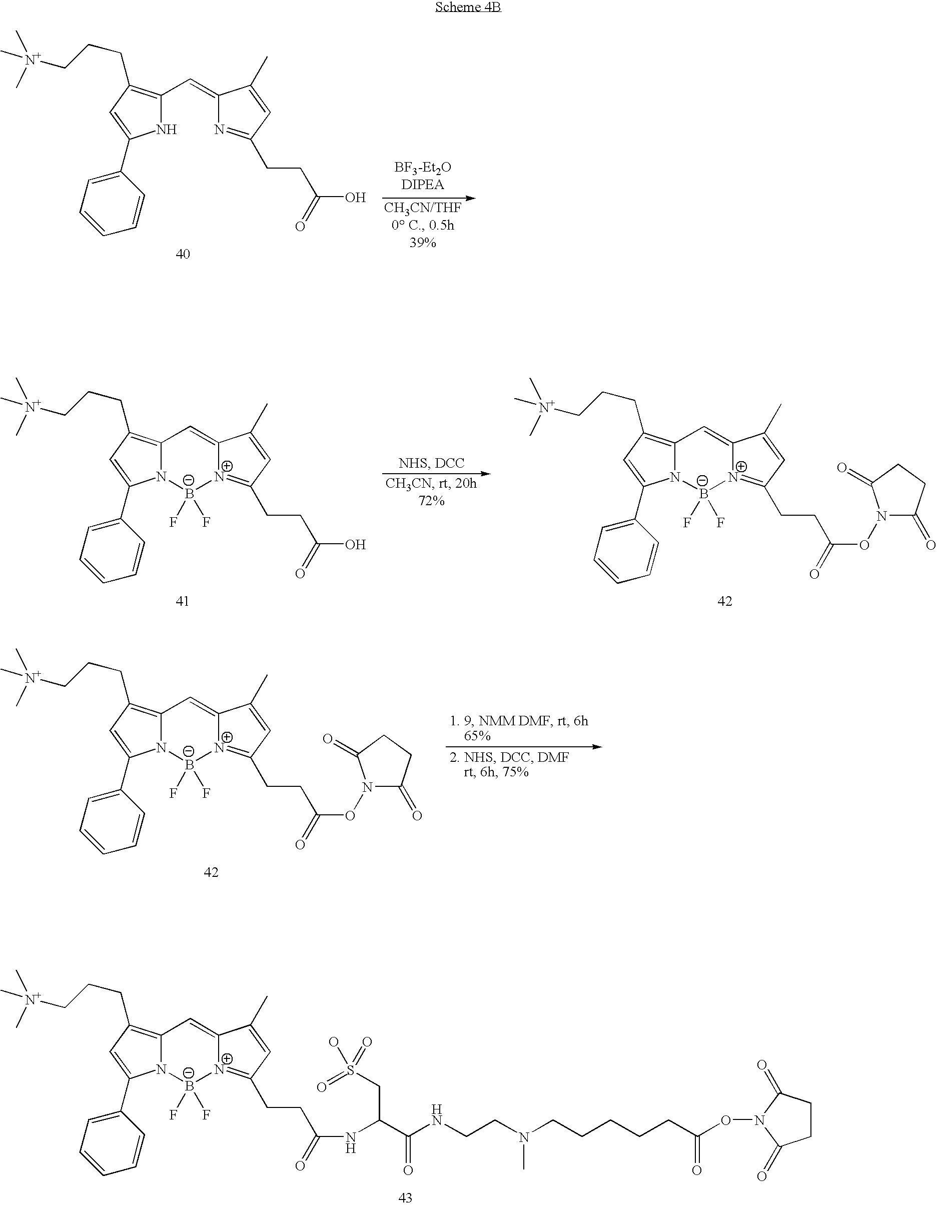

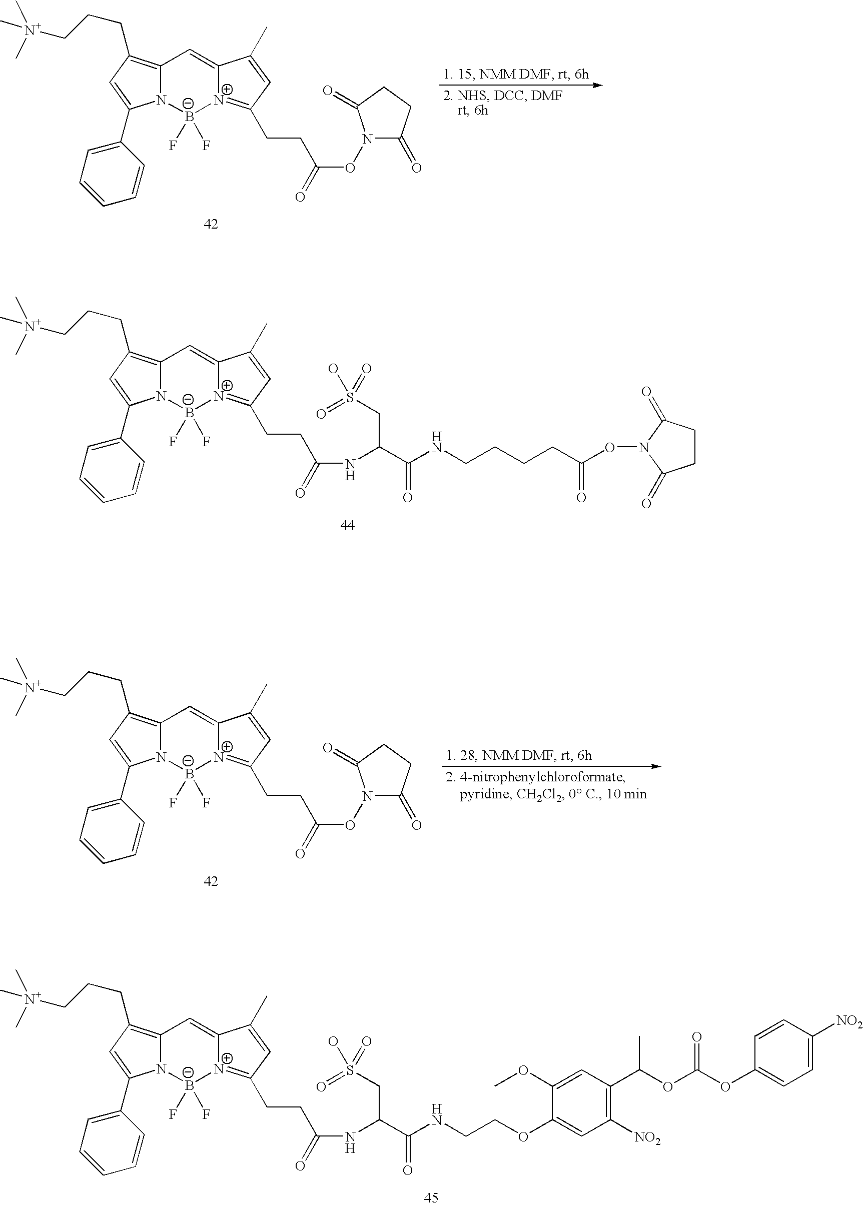

4,4-difluoro-1-methyl-5-phenyl-7-(3-trimethylammonium trifluoroacetate)-propyl-4-bora-3a,4a,diaza-s-indacene-3-propionic acid (41): Diisopropylethylamine (0.15 mL, 0.904 mmol) was added at room temperature to a solution of 5-phenyl-3-(trimethylammonium iodide)-propyl-3′-methyl-5′-(3-propionic acid) dipyrromethene (40) (10 mg, 0.0226 mmol) in anhydrous acetonitrile (3 mL) and anhydrous THF (3 mL) stirred under argon. The mixture was stirred 5 min and cooled to 0° C. BF₃.THF complex (0.02 mL, 0.18 mmol) was added dropwise and the mixture was stirred at 0° C. for 30 min. The solvent was removed in vacuo at 0° C., and the residue purified by reverse phase HPLC to yield a dark red solid (5 mg, 0.00881 mmol, 39%): ¹H NMR (500 MHz, CD₃OD) δ 2.12 (m, 2H), 2.29 (s, 3H), 2.68 (t, 2H, J=7.5 Hz), 2.78 (t, 2H, J=7.5 Hz), 3.09 (s, 9H), 3.15 (t, 2H, J=7.5 Hz), 3.33-3.39 (m, 2H), 6.26 (s, 1H), 6.60 (s, 1H) 7.35-7.44 (m, 3H), 7.55 (s, 1H), 7.84-7.91 (m, 2H); ¹³C NMR (125 MHz, CD₃OD) δ 11.50, 23.28, 25.09, 25.32, 33.52, 53.80, 67.43, 119.63, 120.14, 123.59, 129.32, 130.45, 130.53, 134.23, 135.42, 135.98, 144.46, 145.57, 157.61, 163.16, 175.98; HRMS-EI (m/z): [M] calcd for C₂₅H₃₁BF₂N₃O₂⁺ 454.2477. found 454.2457.

4,4-difluoro-1-methyl-5-phenyl-7-(3-trimethylammonium trifluoroacetate)-propyl-4-bora-3a,4a,diaza-s-indacene-3-propionic acid, succinimidyl ester (42): A solution of 4,4-difluoro-1-methyl-5-phenyl-7-(3-trimethylammonium trifluoroacetate)-propyl-4-bora-3a,4a,diaza-s-indacene-3-propionic acid (41) (13 mg, 0.0229 mmol), NHS (3 mg, 0.0265 mmol) and DCC (6 mg, 0.0292 mmol) in anhydrous acetonitrile (1.2 mL) was stirred at room temperature under argon for 20 hours. The solvent was removed in vacuo at room temperature and the residue purified by reverse phase HPLC to yield a dark red solid (11 mg, 0.0166 mmol, 72%); ¹H NMR (500 MHz, CD₃OD) δ 2.19 (m, 2H), 2.35 (s, 3H), 2.83 (s, 4H), 2.87 (t, 2H, J=7.5 Hz), 3.05 (t, 2H, J=7.5 Hz), 3.14 (s, 9H), 3.28 (t, 2H, J=7.5 Hz), 3.42 (m, 2H), 6.38 (s, 1H), 6.67 (s, 1H), 7.41-7.47 (m, 3H), 7.64 (s, 1H), 7.88-7.93 (m, 2H); ¹³C NMR (125 MHz, CD₃OD) δ 11.53, 23.30, 24.74, 25.09, 23.63, 30.65, 53.79, 67.41, 119.94, 120.27, 123.98, 129.37, 130.56, 130.63, 134.08, 135.75, 135.90, 145.08, 145.44, 158.32, 160.88, 169.60, 171.89; HRMS-EI (m/z): [M] calcd for C₂₉H₃₄BF₂N₄O₄⁺ 551.2641. found 551.2650.

4,4-difluoro-1-methyl-5-phenyl-7-(3-trimethylammonium)-propyl-4-bora-3a,4a,diaza-s-indacene-3-(6-(N-methyl-N-(2-(2-propionamido-3-sulfonate propionamido)ethyl)amino))hexanoic acid, succinimidyl ester hydrotrifluoroacetate (43): N-methylmorpholine (0.146 mL, 1.36 mmol) was added under argon to a solution of 4,4-difluoro-1-methyl-5-phenyl-7-(3-trimethylammonium trifluoroacetate)-propyl-4-bora-3a,4a,diaza-s-indacene-3-propionic acid, succinimidyl ester (42) (40 mg, 0.068 mmol) and 6-(N-methyl-N-(2-(2-amino-3-sulfonate-propionamido)ethyl)amino)hexanoic acid, hydroacetate (9) (105 mg, 0.273 mmol) in anhydrous DMF (4 mL). The mixture was stirred under argon at room temperature for 6 hours. The solvent was removed in vacuo at room temperature, and the residue purified by reverse phase HPLC to yield a dark red solid (39 mg, 0.044 mmol, 65%); ¹H NMR (500 MHz, CD₃OD) δ 1.41 (m, 2H), 1.66 (sextet, 2H, J=7 Hz), 1.77 (m, 2H), 2.22 (m, 2H), 2.32 (q, 2H, J=7 Hz), 2.38 (s, 3H), 2.68 ((9), 2H, J=7.5 Hz), 2.87 (s, 3H), 2.90 (t, 2H, J=7.5 Hz), 3.20 (dt, 1H, J¹=8.5 Hz, J²=12 Hz), 3.08-3.74 (m, 11H), 3.16 (s, 9H), 4.64 (m, 1H), 6.32 (s, 1H), 6.66 (s, 1H), 7.43 (m, 3H) 7.63 (s, 1H), 7.90 (m, 2H); HRMS-ES (m/z): [M] calcd for C₃₅H₅₄BF₂N₆O₇S⁺ 775.3836. found 775.3834. Then, the resulting dark red solid (12 mg, 13.5 μmol), was added to NHS (15.5 mg, 0.135 mmol) and DCC (28 mg, 0.135 mmol) in anhydrous DMF (0.75 mL) and stirred at room temperature under argon for 10 hours. The DMF was removed in vacuo at room temperature, and the residue purified by reverse phase HPLC to yield a dark red solid (10 mg, 10.1 μmol, 75%); ¹H NMR (500 MHz, CD₃CN) δ 1.44 (m, 2H), 1.73 (sex, 4H, J=7 Hz), 1.97 (m, 2H), 2.11 (m, 2H), 2.35 (s, 3H), 2.60-2.65 (m, 2H), 2.65 (t, 2H, J=7.5 Hz), 2.70-3.37 (m, 17H), 3.02 (s, 9H), 3.63-3.73 (m, 1H), 3.78-3.90 (m, 1H), 4.55 (br, 1H), 6.33 (s, 1H), 6.61 (s, 1H), 7.21 (br, 1H), 7.46-7.51 (m, 3H), 7.55 (s, 1H), 7.88 (d, 2H, J=6.5 Hz); FIRMS-ES (m/z): [M] calcd for C₄₁H₅₇BF₂N₇O₉S⁺ 872.4000. found 872.4044.

(45). N-methylmorpholine (6 μL, 0.054 mmol) was added at room temperature to a solution of 4,4-difluoro-1,5-dimethyl-7-(3-trimethylammonium trifluoroacetate)-propyl-4-bora-3a,4a,diaza-s-indacene-3-propionic acid, succinimidyl ester (42) (13 mg, 0.0216 mmol) and 28 (13 mg, 0.0319 mmol) in anhydrous DMF (0.3 mL). The mixture was stirred at room temperature under argon for 3.5 hours. The solvent was lyophilized off and the resulting solid purified by reverse phase HPLC to yield an orange solid (7 mg, 0.0897 mmol, 42%): ¹H NMR (500 MHz, CD₃CN:D₂O (1:1) 6) 1.39 (d, 3H, J=6 Hz), 2.00 (m, 2H), 2.22 (s, 3H), 2.43 (s, 3H), 2.60 (t, 2H, J=7.5 Hz), 2.66 (t, 2H, J=7.5 Hz), 2.99 (s, 9H), 3.01-3.14 (m, 4H), 3.23 (m, 2H), 3.52 (t, 2H, J=5.5 Hz), 3.90 (s, 3H), 4.04 (q, 2H, J=5 Hz), 4.53 (dd, 1H, J1=4.5 Hz, J2=8 Hz), 5.34 (q, 1H, J=6.5 Hz), 6.22 (s, 1H), 6.23 (s, 1H), 7.30 (s, 1H), 7.39 (s, 1H), 7.52 (s, 1H); FIRMS-ES (m/z): [M+H] calcd for C₃₄H₄₈BF₂N₆O₁₀S 781.3214. found 781.3204. Then p-nitrophenylchloroformate was added dropwise to a solution of the resulting orange solid (7 mg, 0.00897 mmol) and N-methylmorpholine (8 μL, 0.0718 mmol) in anhydrous acetonitrile (0.4 mL) at 0° C. The mixture was stirred under argon at 0° C. for 2 hours and brought to room temperature. Stirring was continued for 24 hours. The solvent was lyophilized off and the resulting solid purified by reverse phase HPLC to yield an orange solid (2.5 mg, 0.0264 mmol, 29% (69% BRSM)): ¹H NMR (500 MHz, CD₃CN δ) 1.71 (d, 3H, J=6 Hz), 2.02 (m, 2H), 2.25 (s, 3H), 2.47 (s, 3H), 2.62 (t, 2H, J=7.5 Hz), 2.68 (t, 2H, J=7.5 Hz), 2.99 (s, 9H), 3.02 (m, 2H), 3.12 (t, 2H, J=7 Hz), 3.24, (m, 2H), 3.55 (q, 2H, J=5.5 Hz), 3.96 (s, 3H), 4.10 (t, 2H, J=5.5 Hz), 4.63 (m, 1H), 6.21 (s, 1H), 6.27 (s, 1H), 6.33 (q, 1H, J=6.5 Hz), 7.15 (s, 1H), 7.36 (d, 2H, J=9 Hz), 7.37 (s, 1H), 7.58 (s, 1H), 7.62-7.74 (br, 2H), 8.22 (d, 2H, J=9 Hz); HRMS-ES (m/z): [M+H] calcd for C₄₁H₅₁BF₂N₇O₁₄S 946.3278. found 946.3262.

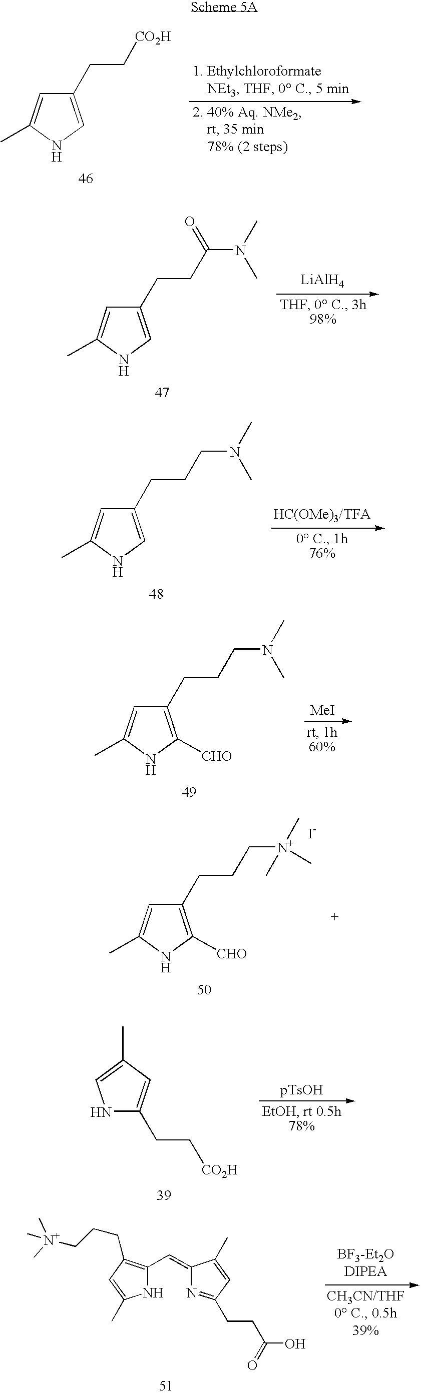

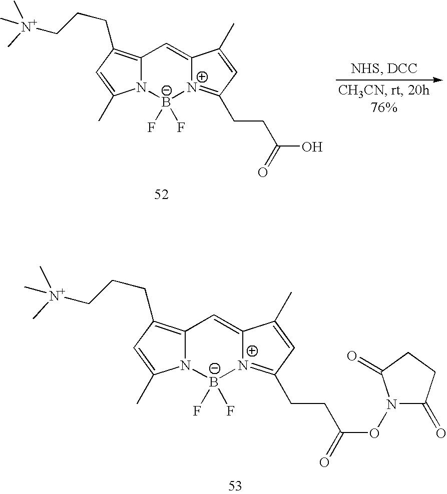

N,N-dimethyl-3-(5-methyl-1H-3-pyrrolyl)propanamide (47): Ethyl chloroformate (0.143 mL, 1.5 mmol) was added drop-wise to a stirred solution of 3-(5-methyl-1H-3-pyrrolyl)propanoic acid (46) (230 mg, 1.5 mmol) and triethylamine (0.208 mL, 1.5 mmol) in anhydrous THF (2 mL) at 0° C. The mixture was stirred at 0° C. under argon for 5 min and 40% aqueous dimethylamine was added (0.400 mL of 3.24 mmol). The ice bath was removed and the mixture was stirred at room temperature for 35 min. The THF was removed in vacuo and the resulting residue partitioned between dilute aqueous NaHCO₃ and EtOAc. The EtOAc extract was dried over Na₂SO₄ and evaporated in vacuo to yield an off white crystalline solid (211 mg, 1.17 mmol, 78): ¹H NMR (500 MHz, CDCl₃) δ 2.25 (s, 3H), 2.61 (m, 2H), 2.82 (m, 2H), 2.90 (s, 3H), 3.08 (s, 3H), 5.78 (s, 1H), 6.45 (s, 1H), 8.78 (s, 1H); ¹³C NMR (125 MHz, CDCl₃) δ 12.73, 22.70, 35.16, 35.20, 37.04, 105.72, 113.33, 122.55, 127.61, 173.03; HRMS-ES (m/z): [M+H] calcd for C₁₀H₁₆N₂O 181.1335. found 181.1309.

N,N-dimethyl-3-(5-methyl-1H-3-pyrrolyl)propan-1-amine (48): To a suspension of lithium aluminum hydride (220 mg, 5.8 mmol) in anhydrous THF (14 mL) maintained at 0° C. was added a solution of N,N-dimethyl-3-(5-methyl-1H-3-pyrrolyl)propanamide (47) (210 mg, 1.16 mmol) in anhydrous THF (28 mL). The mixture was then stirred under argon at room temperature for 3 hours. The reaction was quenched by addition of aqueous Na₂CO₃ (1 M) and the mixture was extracted with EtOAc. The combined organic extracts were washed with brine and dried over Na₂SO₄. The solvent was removed in vacuo to yield a yellow oil (189 mg, 1.14 mmol, 98%): ¹H NMR (500 MHz, CD₃OD) δ 1.71 (m, 2H), 2.16 (s, 3H), 2.20 (s, 6H), 2.32 (m, 2H) 2.39 (t, 2H, J=7.5 Hz), 5.63 (s, 1H), 6.32 (s, 1H); ¹³C NMR (125 MHz, CD₃OD) δ 13.17, 26.17, 29.99, 45.55, 60.61, 106.75, 114.35, 124.02, 128.44; HRMS-ES (m/z): [M+H] calcd for C₁₀H₁₈N₂ 167.1543. found 167.1536.

3-(3-(dimethylamino)-propyl)-5-methyl-4H-pyrrole-2-carboxaldehyde (49): Trimethyl orthoformate (1.9 mL, 17.6 mmol) was added to a stirred solution of N,N-dimethyl-3-(5-methyl-1H-3-pyrrolyl)propan-1-amine (48) (885 mg, 5.33 mmol) in TFA (20 mL) stirred under argon at 0° C. Stirring was continued at 0° C. for 1 h. Cold water was added and the mixture was basified to ph 12 with aqueous NaOH. The mixture was extracted with EtOAc (×3) and the combined organic extracts were dried over Na₂SO₄. The solvent was removed in vacuo and the resulting residue purified by flash chromatography on DAVISIL (20% MeOH, 80% CH₂Cl₂) to yield a yellow oil (786 mg, 4.05 mmol, 76%): ¹H NMR (500 MHz, CD₃OD) δ 1.46 (p, 2H, J=7.5 Hz), 1.89 (s, 6H), 1.94 (s, 3H), 2.02 (t, 2H, J=7.5 Hz), 2.39 (t, 2H, J=7.5 Hz), 5.56 (s, 9.05 (s, 1H); HRMS-ES (m/z): [M+H] calcd for C₁₁H₁₈N₂O 195.1492. found 195.1469.

### Trimethyl-[3-(2-formyl-5-methyl-1H-3-pyrrolyl)-propyl]-ammonium triflate (50)

Methyl iodide was added to a solution of 3-(3-(dimethylamino)-propyl)-5-methyl-1H-pyrrole-2-carboxaldehyde (49) (788 mg, 4.06 mmol) in THF (20 mL) stirred under argon. The mixture was stirred 1 hour at room temperature. The solvent was removed in vacuo, to yield a light brown solid (1.36 g, 4.06 mmol, 100%): ¹H NMR (500 MHz, CD₃OD) δ 2.12 (m, 2H), 2.27 (s, 3H), 2.83 (t, 2H, J=7.5 Hz), 3.13 (s, 9H), 3.38 (m, 2H), 6.02 (s, 1H), 9.44 (s, 1H); HRMS-ES (m/z): [M] calcd for C₁₂H₂₁N₂O⁺ 209.1648. found 209.1667.

5-methyl-3-(3-trimethylammonium trifluoroacetate)-propyl-3′-methyl-5′-(3-propionic acid)dipyrromethene (51): p-TsOH monohydrate (325 mg, 1.71 mmol) was added to a solution of trimethyl-[3-(2-formyl-4-methyl-1H-3-pyrrolyl)-propyl]-ammonium triflate (50) (550 mg, 1.71 mmol) and 3-(4-methyl-1H-2-pyrrolyl)-propanoic acid (39) (309 mg, 1.71 mmol) in ethanol (6 mL)). The mixture was stirred 30 min at room temperature. The reaction mixture was then passed through a DOWEX 21K Cl anion exchange resin and eluted with water. The eluate was evaporated in vacuo and purified by reverse phase HPLC to yield an orange solid (610 mg, 1.33 mmol, 78%): ¹H NMR (500 MHz, D₂O) δ 2.01 (m, 2H), 2.15 (s, 3H), 2.32 (s, 3H), 2.64 (t, 2H, 7.5 Hz), 2.65 (t, 2H, 7.5 Hz), 2.86 (t, 2H, 7.5 Hz), 2.99 (s, 9H), 3.22 (m, 2H), 6.20 (s, 1H), 6.30 (s, 1H), 7.04 (s, 1H); ¹³C NMR (125 MHz, D₂O) δ 11.26, 13.48, 22.08, 22.99, 23.21, 52.88, 52.97, 65.64, 116.69, 121.06, 126.98, 148.25, 148.29, 149.20, 156.08, 157.11, 162.47, 176.05; HRMS-ES (m/z): [M] calcd for C₂₀H₃₀N₃O₂⁺ 344.2333. found 344.2313.

4,4-difluoro-1,5-dimethyl-7-(3-trimethylammonium trifluoroacetate)-propyl-4-bora-3a,4a,diaza-s-indacene-3-propionic acid (52): Diisopropylethylamine (1 mL, 5.76 mmol) was added at room temperature to a solution of 5-methyl-3-(trimethylammonium trifluoroacetate)-propyl-3′-methyl-5′-(3-propionic acid)dipyrromethene (51) (88 mg, 0.192 mmol) in acetonitrile (9 mL) stirred under argon. The mixture was stirred 5 min and cooled to 0° C. BF₃.THF complex (0.127 mL, 1.15 mmol) was added dropwise and the mixture was stirred at 0° C. for 30 min. The solvent was removed in vacuo at 0° C., and the residue purified by reverse phase HPLC to yield an orange solid (38 mg, 0.0749 mmol, 39%): ¹H NMR (500 MHz, CD₃CN) δ 2.03 (m, 2H), 2.29 (s, 3H), 2.49 (s, 3H), 2.69 (t, 2H, J=7.5 Hz), 2.71 (t, 2H, J=8 Hz), 3.01 (s, 9H), 3.12 (t, 2H, J=7.5 Hz), 3.26 (m, 2H), 6.25 (s, 2H), 7.43 (s, 1H); ¹³C NMR (125 MHz, CD₃CN) δ 11.63, 15.04, 22.93, 24.91, 33.30, 54.08, 66.97, 118.86, 119.33, 123.31, 133.72, 134.66, 144.11, 145.41, 157.77, 161.10, 174.53; HRMS-ES (m/z): [M] calcd for C₂₀H₂₉BF₂N₃O₂⁺ 392.2319. found 392.2330.

4,4-difluoro-1,5-dimethyl-7-(3-trimethylammonium trifluoroacetate)-propyl-4-bora-3a,4a,diaza-s-indacene-3-propionic acid, succinimidyl ester (53): A solution of 4,4-difluoro-1,5-dimethyl-7-(3-trimethylammonium trifluoroacetate)-propyl-4-bora-3a,4a,diaza-s-indacene-3-propionic acid (52) (10 mg, 0.0198 mmol), NHS (14 mg, 0.119 mmol) and DCC (25 mg, 0.119 mmol) in anhydrous DMF (0.5 mL) was stirred at room temperature under argon for 5 hours. The DMF was removed in vacuo at room temperature, and the residue purified by reverse phase HPLC to yield an orange solid (9 mg, 0.015 mmol, 76%): ¹H NMR (500 MHz, CD₃CN) δ 2.04 (m, 2H), 2.30 (s, 3H), 2.50 (s, 3H), 2.72 (t, 2H, J=7.2 Hz), 2.77 (s, 4H), 3.02 (s, 3H), 3.05 (t, 2H, J=7.2 Hz), 3.24 (t, 2H, J=7.2 Hz), 3.28 (m, 2H), 6.28 (s, 2H), 7.49 (s, 1H); ¹³C NMR (150 MHz, CD₃CN) δ 11.79, 15.27, 23.28, 24.65, 25.12, 26.78, 30.88, 54.48, 67.43, 119.17, 120.09, 123.84, 134.48, 134.87, 144.00, 146.39, 158.50, 159.26, 169.50, 171.17; HRMS-ES (m/z): [M] calcd for C₂₄H₃₂BF₂N₄O₄⁺ 489.2484. found 489.2496.

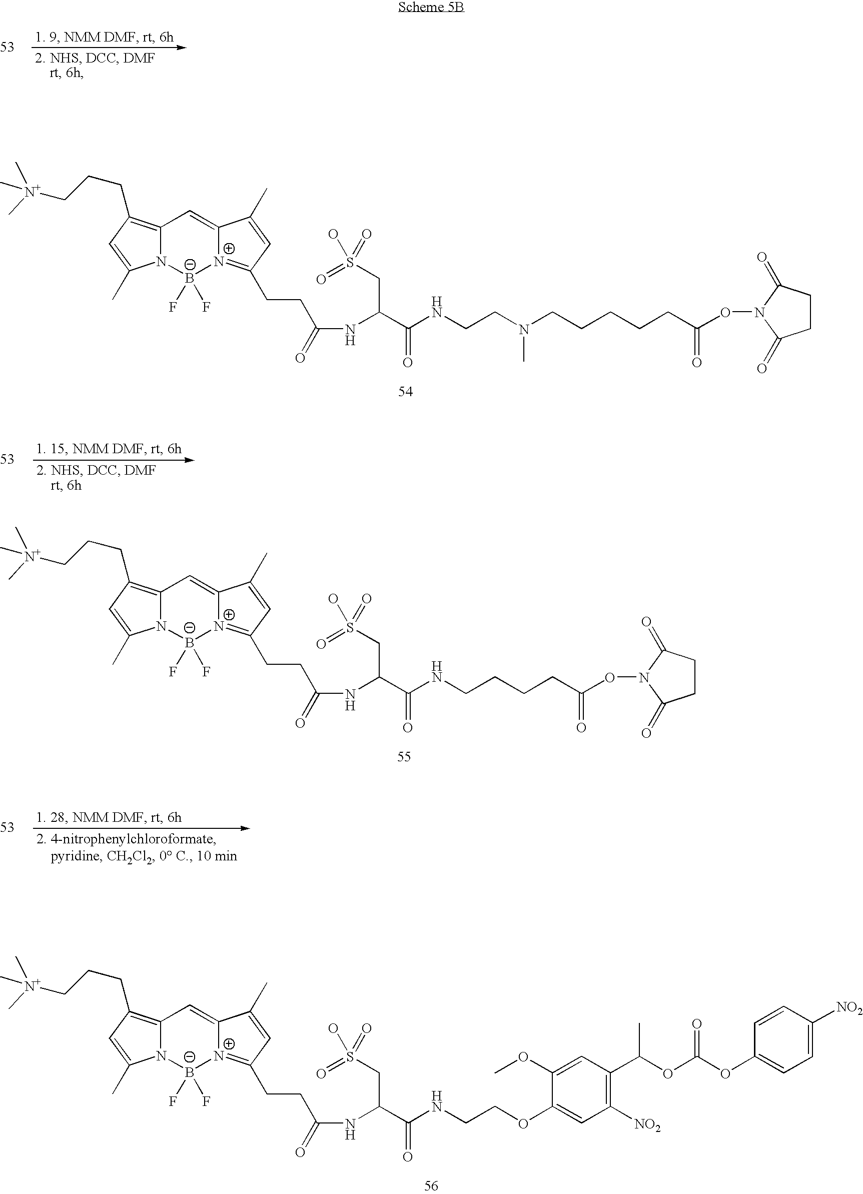

4,4-difluoro-1,5-dimethyl-7-(3-trimethylammonium)-propyl-4-bora-3a,4a,diaza-s-indacene-3-(6-(N-methyl-N-(2-(2-propionamido-3-sulfonate-propionamido)ethyl)amino))hexanoic acid, succinimidyl ester hydrotrifluoroacetate (54): N-methylmorpholine (0.287 mL, 2.62 mmol) was added under argon to a solution of 4,4-difluoro-1,5-dimethyl-7-(3-trimethylammonium)-propyl-4-bora-3a,4a,diaza-s-indacene-3-propionic acid, succinimidyl ester (53) (79 mg, 0.131 mmol) and 6-(N-methyl-N-(2-(2-amino-3-sulfonate-propionamido)ethyl)amino)hexanoic acid, hydroacetate (9) (200 mg, 0.524 mmol) in anhydrous DMF (2 mL). The mixture was stirred under argon at room temperature for 3.5 hours. The solvent was removed in vacuo at room temperature, and the residue purified by reverse phase HPLC to yield an orange solid (83 mg, 0.101 mmol, 77%); ¹H NMR (500 MHz, CD₃CN:D₂O (2:1)) δ 1.28 (p, 2H, J=8 Hz), 1.54 (p, 2H, J=7.5 Hz), 1.62 (m, 2H), 2.00 (m, 2H), 2.23 (s, 3H), 2.27 (t, 2H J=7.5 Hz), 2.43 (s, 3H), 2.62 (q, 2H, J=8.2 Hz), 2.67 (t, 2H, J=7.5 Hz), 2.75 (d, 3H, J=3 Hz), 2.90-3.30 (m, 10H), 2.98 (s, 9H), 3.51 (m, 2H), 4.55 (m, 1H), 6.21 (s, 1H), 6.25 (s, 1H), 7.42 (s, 1H); ¹³C NMR (150 MHz, CD₃CN:D₂O (2:1)) δ 11.67, 15.09, 22.90, 24.12, 24.58, 24.89, 24.96, 26.40, 34.49, 34.93, 35.38, 41.11, 51.87, 52.45, 54.14, 56.58, 57.45, 67.03, 119.12, 119.61, 123.11, 133.82, 134.64, 144.64, 145.94, 158.37, 160.51, 173.39, 174.52, 178.10; HRMS-ES (m/z): [M] calcd for C₃₂H₅₂BF₂N₆O₇S⁺ 713.3680. found 713.3652.

The orange solid was then added to NHS (6.3 mg, 54.4 μmol) and DCC (11.2 mg, 54.4 μmol) in anhydrous DMF (0.2 mL) and stirred at room temperature under argon for 23 hours. The DMF was removed in vacuo at room temperature, and the residue purified by reverse phase HPLC to yield an orange solid (4.3 mg, 4.68 μmol, 86%); ¹H NMR (500 MHz, CD₃CN:D₂O (1:1)) δ 1.25-1.40 (m, 2H), 1.54 (p, 2H, J=7.5 Hz), 1.64 (m, 2H), 2.00 (m, 2H), 2.23 (s, 3H), 2.27 (t, 2H, J=7.5 Hz), 2.44 (s, 3H), 2.60-2.65 (m, 2H), 2.61 (s, 4H), 2.67 (t, 2H, J=7.5

Hz), 2.76 (d, 3H, J=2.5 Hz), 2.91-3.30 (m, 10H), 2.95 (s, 9H), 3.52 (m, 2H), 4.55 (m, 1H), 6.21 (s, 1H), 6.25 (s, 1H), 7.42 (s, 1H); ¹³C NMR (150 MHz, CD₃CN:D₂O (1:1)) δ 10.99, 14.41, 22.13, 23.40, 23.94, 24.20, 25.50, 25.68, 25.82, 33.74, 34.13, 34.50, 40.28, 51.01, 51.72, 53.32, 55.82, 56.58, 66.16, 118.15, 118.86, 122.57, 133.04, 133.88, 143.86, 145.18, 157.47, 159.83, 172.57, 173.53, 175.27, 177.22; HRMS-ES (m/z): [M] calcd for C₃₆H₅₅BF₂N₇O₉S⁺ 810.3844. found 810.3841.

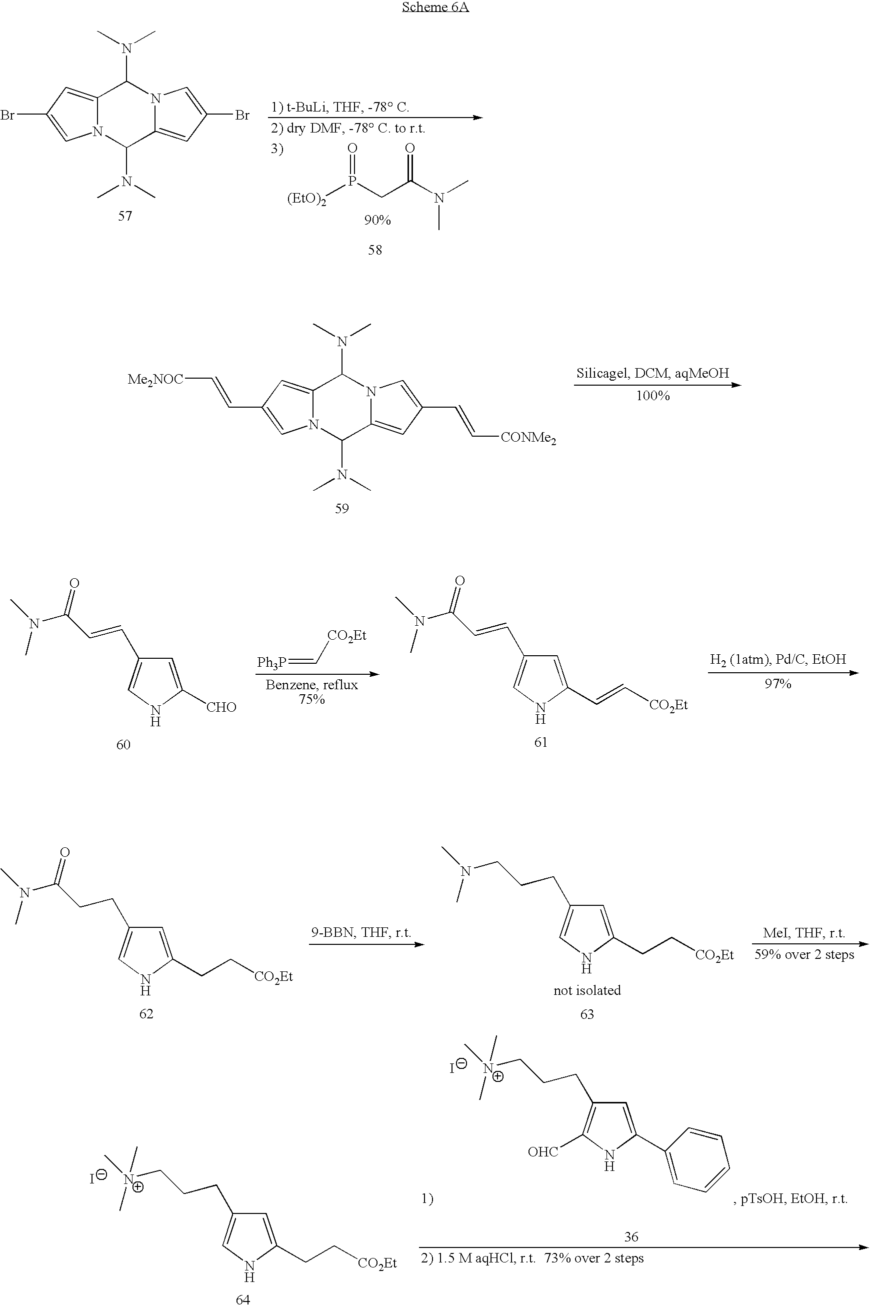

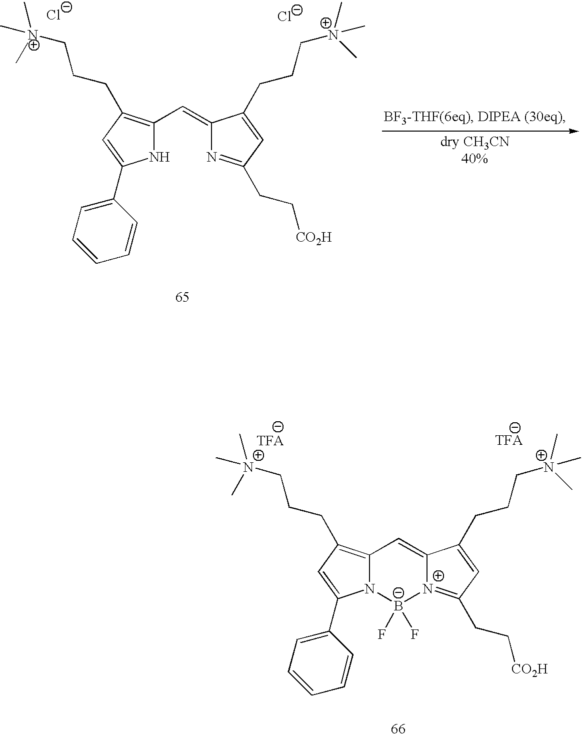

Dimer of 3-(3-N,N-dimethylacrilamidyl)-6-dimethylamino-1-azafulvene (59): 1.7 M t-Butyl lithium in pentane (5.86 mL, 9.96 mmol) was added drop-wise to a stirred solution of the dimer of 3-bromo-6-dimethylamino-1-azafulvene (57) (1 g, 2.49 mmol) in anhydrous THF (100 mL) at −78° C. under argon. The mixture was stirred at −78° C. for 30 min and anhydrous DMF (0.385 mL, 4.98 mmol) was added drop-wise. Stirring was continued at −78° C. for 1 hour and the dry ice bath was removed. The temperature was allowed to rise to 15° C. over 1 hour. The mixture was cooled to 5° C. in an ice bath and a solution of diethyl (dimethylcarbamoyl)methylphosphonate (58) (1.11 g, 4.98 mmol) in anhydrous THF (5 mL) was added drop-wise. The ice bath was removed after 5 min and the mixture was stirred at room temperature under argon for 20 hours. The resulting precipitate was filtered out and dried in vacuo to yield the desired product as an off white powder (700 mg). Water was added to the liquor of filtration and the resulting solution was extracted with ethyl acetate. The combined organic extracts were dried oven Na₂SO₄ and evaporated in vacuo to yield a red oil (450 mg) which was used as such in the next step. Mp 193-200° C. (decomposed); ¹H NMR (300 MHz, CDCl₃) δ 2.30 (s, 6H), 3.07 (s, 3H), 3.16 (s, 3H), 5.84 (s, 1H), 6.44 (s, 1H), 6.60 (d, 1H, J=15 Hz), 7.15 (s, 1H), 7.63 (d, 1H, J=15 Hz); ¹³C NMR (125 MHz, CDCl₃) δ 36.04, 37.52, 39.45, 72.23, 104.03, 113.42, 121.69, 122.56, 127.26, 136.36, 167.64; HRMS-ES (m/z): [(M+2H)/2] calcd for C₂₄H₃₄N₆O₂ 220.1444. found 220.1432.

(E)-3-(5-formyl-1H-pyrrol-3-yl)-N,N-dimethylacrylamide (60): The red oil (59) (450 mg) obtained in the previous step was dissolved in DCM (10 mL). MeOH (95%, 2 mL) was added to the flask followed by silica gel (200 mg). The mixture was stirred at room temperature overnight. The silica gel was filtered out of the resulting suspension and rinsed with a methanol:DCM solution (1:1). The liquor of filtration was evaporated in vacuo and the resulting oil purified by DAVISIL flash chromatography (2% MeOH in DCM) to yield a yellow solid (245 mg, 1.28 mmol). The clean dimer of 3-(3-N,N-dimethylacrilamidyl)-6-dimethylamino-1-azafulvene (700 mg, 1.59 mmol) obtained in the previous step was subjected to the same conditions in a different flask. The silica gel was filtered out of the resulting suspension and rinsed with a methanol:DCM solution (1:1). The resulting solution was evaporated to dryness to yield the desired product as a yellow solid (610 mg, 3.19 mmol). Combined with the 245 mg obtained after chromatography the reaction leads to 855 mg of product (855 mg, 4.47 mmol, 90% over two steps): ¹H NMR (500 MHz, CDCl₃) δ 3.00 (s, 3H), 3.10 (s, 3H), 6.30 (d, 1H, J=15 Hz), 7.11 (s, 1H), 7.30 (s, 1H), 7.57 (d, 1H, J=15 Hz), 9.47 (s, 1H); ¹³C NMR (125 MHz, CDCl₃) δ 36.04, 37.45, 114.70, 118.22, 123.15, 128.13, 133.80, 135.30, 167.34, 179.79; HRMS-ES (m/z): [M+H] calcd for C₁₀H₁₂N₂O₂ 193.0972. found 193.0963.

(E)-ethyl 3-(4-((E)-2-(dimethylcarbamoyl)vinyl)-1H-pyrrol-2-yl)acrylate (61): A solution of (E)-3-(5-formyl-1H-pyrrol-3-yl)-N,N-dimethylacrylamide (60) (34 mg, 0.177 mmol) and (carbethoxymethylene) triphenylphosphorane (92 mg, 0.266 mmol) in anhydrous benzene (2 mL) was refluxed under argon for 22 hours. The solvent was removed in vacuo and the resulting solid purified by DAVISIL flash chromatography (2% MeOH in DCM) to yield a white powder (35 mg, 0.134 mmol, 75%): ¹H NMR (500 MHz, d6-DMSO) δ 1.23 (t, 3H, J=7.2 Hz), 2.90 (s, 3H), 3.10 (s, 3H), 4.15 (q, 2H, J=7.2 Hz), 6.26 (d, 1H, J=16 Hz), 6.76 (d, 1H, J=15 Hz), 6.96 (s, 1H), 7.35 (d, 1H, J=15 Hz), 7.40 (d, 1H, J=16 Hz), 7.43 (s, 1H); ¹³C NMR (150 MHz, d6-DMSO) δ 13.72, 59.04, 111.30, 112.05, 113.76, 122.09, 124.72, 129.12, 133.47, 134.28, 166.06; HRMS-ES (m/z): [M+H] calcd for C₁₄H₁₈N₂O₃ 263.1390. found 263.1374.

Ethyl 3-(4-(2-(dimethylcarbamoyl)ethyl)-1H-pyrrol-2-yl)propanoate (62): (E)-ethyl 3-(4-((E)-2-(dimethylcarbamoyl)vinyl)-1H-pyrrol-2-yl)acrylate (61) (270 mg, 1.03 mmol) was dissolved in absolute ethanol (20 mL). 10% Pd on carbon (57 mg, 0.0538 mmol) was added to the solution and the resulting suspension was stirred under hydrogen (1 atm) for 6 hours. The catalyst was filtered out and rinsed with ethanol. The filtrate was evaporated in vacuo to yield a yellow oil (266 mg, 97%): ¹H NMR (500 MHz, CDCl₃) δ 1.25 (t, 3H, J=7.0 Hz), 2.56 (t, 2H, J=7.9 Hz), 2.61 (t, 2H, J=7.0 Hz), 2.77 (t, 2H, J=7.9 Hz), 2.86 (t, 2H, J=7.0 Hz), 2.95 (s, 3H), 2.97 (s, 3H), 4.14 (q, 2H, J=7.0 Hz), 5.79 (s, 1H), 6.46 (s, 1H) 8.53 (br, 1H); ¹³C NMR (125 MHz, CDCl₃) δ 14.01, 22.67, 22.70, 34.29, 35.09, 35.17, 37.05, 60.28, 105.28, 113.83, 122.38, 130.86, 172.94, 173.43; HRMS-ES (m/z): [M+H] calcd for C₁₄H₂₂N₂O₃ 267.1703. found 267.1731.

Ethyl 3-(4-(3-(dimethylamino)propyl)-1H-pyrrol-2-yl)propanoate (63): 9-BBN (0.5M in THF, 4.7 mL, 2.36 mmol) was added drop-wise to a stirred solution of ethyl 34442-(dimethylcarbamoyl)ethyl)-1H-pyrrol-2-yl)propanoate (62) (285 mg, 1.07 mmol) in anhydrous THF (8 mL) under argon. The mixture was stirred at room temperature under an argon atmosphere for 3 hours. Ethanolamine (0.142 mL, 2.36 mmol) was added to the solution and the solvent was removed in vacuo. The flask was filled with pentane and the resulting suspension stirred 2 hours at room temperature. The flask was then placed at 0° C. overnight. The solid was filtered out and rinsed with ice cold pentane. The filtrate was evaporated in vacuo to yield the crude product as a yellowish oil (232 mg) which was used as such in the next step. HRMS-ES (m/z): [M+H] calcd for C₁₄H₂₄N₂O₂ 253.1911. found 253.1910.

### Trimethyl-[3-(ethyl 3-(1H-pyrrol-2-yl)propanoate)-propyl]-ammonium iodide (64)

To a solution of crude ethyl 3-(4-(3-(dimethylamino)propyl)-1H-pyrrol-2-yl)propanoate (63) (56 mg) in dry THF (1 mL) under argon was added methyl iodide (0.8 mL). The mixture was stirred at room temperature for one hour. The precipitated product was filtered out to yield an off white powder (60 mg, 0.225 mmol, 60% over 2 steps): ¹H NMR (500 MHz, CD₃OD) δ 1.23 (t, 3H, J=7.2 Hz), 2.01 (m, 2H), 2.52 (t, 2H, J=7.0 Hz), 2.58 (t, 2H, J=7.5 Hz), 2.83 (t, 2H, J=7.5 Hz) 3.00-3.15 (m, 2H), 3.11 (s, 9H), 4.11 (q, 2H, J=7.2 Hz), 5.74 (s, 1H), 6.45 (s, 1H); HRMS-EI (m/z): [M] calcd for C₁₅H₂₇N₂O₂⁺ 267.2067. found 267.2070.

5-phenyl-3,3′-(3,3-bistrimethylammonium trifluoroacetate)propyl-5′-(3-propionic acid) dipyrromethene (65): p-TsOH monohydrate (29 mg, 0.152 mmol), was added to a stirred solution of trimethyl-[3-(ethyl 3-(1H-pyrrol-2-yl)propanoate)-propyl]ammonium iodide (64) (60 mg, 0.152 mmol) and trimethyl-[3-(2-formyl-5-phenyl-1H-3-pyrrolyl)-propyl]-ammonium iodide (36) (61 mg, 0.152 mmol) in absolute ethanol (9 mL). The mixture was stirred 80 min at room temperature. The reaction mixture was then diluted with water (10 mL) and passed through a DOWEX 21K Cl anion exchange resin and eluted with water. When the wash from the column came out clean elution was stopped. The eluate was evaporated in vacuo to yield a red solid (97 mg). The resulting red solid was stirred at room temperature in 1M aqueous HCl (3 mL) for 15 hours. The solvent was removed in vacuo and the residue purified by reverse phase HPLC to yield a red solid (80 mg, 0.111 mmol, 73% over 2 steps): ¹H NMR (500 MHz, CD₃CN) δ 2.10 (m, 2H), 2.16 (m, 2H), 2.79 (t, 2H, J=7 Hz), 2.82 (t, 2H, J=7 Hz), 2.90 (t, 2H, J=7.5 Hz), 3.04 (s, 18H), 3.10 (t, 2H, J=7.5 Hz), 3.21-3.38 (m, 4H), 6.57 (s, 1H), 7.04 (s, 1H), 7.31 (s, 1H), 7.45-7.58 (m, 3H), 8.00 (dd, 2H, J1=8 Hz, J2=2 Hz); HRMS-ES (m/z): [M/2] calcd for C₃₀H₄₄N₄O₂²⁺ 246.1727. found 246.1742.

4,4-difluoro-1,7-(3,3-bistrimethylammonium trifluoroacetate)propyl-5-phenyl-4-bora-3a,4a,diaza-s-indacene-3-propionic acid (66): Diisopropylethylamine (0.581 mL, 3.34 mmol) was added at room temperature to a solution of 5-phenyl-3,3′-(3,3-bistrimethylammonium trifluoroacetate)propyl-5′-(3-propionic acid) dipyrromethene (65) (80 mg, 0.111 mmol) in anhydrous acetonitrile (5 mL) stirred under argon. The mixture was stirred 5 min and cooled to 0° C. BF₃.THF complex (0.073 mL, 0.666 mmol) was added dropwise and the mixture was stirred at 0° C. for 30 min. The solvent was removed in vacuo at 0° C., and the residue purified by reverse phase HPLC to yield recovered starting material (30 mg, 0.0418 mmol) together with a red solid (40 mg, 0.0522 mmol, 47% (75% BRSM)): ¹H NMR (500 MHz, CD₃CN) δ 2.05-2.20 (m, 4H), 2.71 (t, 2H, J=7.3 Hz), 2.80 (t, 2H, J=7.3 Hz), 2.84 (t, 2H, J=7.3 Hz), 3.03 (s, 9H), 3.04 (s, 9H), 3.13 (t, 2H, J=7.3 Hz), 3.25-3.40 (m, 4H), 6.47 (s, 1H), 6.66 (s, 1H), 7.43-7.56 (m, 3H), 7.64 (s, 1H), 7.90 (dd, 2H, J1=7.8 Hz, J2=1.8 Hz); ¹³C NMR (500 MHz, CD₃CN) δ 23.25, 24.77, 25.08, 25.51, 33.69, 54.28, 54.31, 67.14, 119.26, 120.31, 124.81, 129.68, 130.57, 130.92, 134.05, 134.85, 135.69, 145.64, 147.29, 157.84, 163.19, 174.95; HRMS-ES (m/z): [M/2] calcd for C₃₀H₄₃BF₂N₄O₂²⁺ 270.1721. found 270.1721.

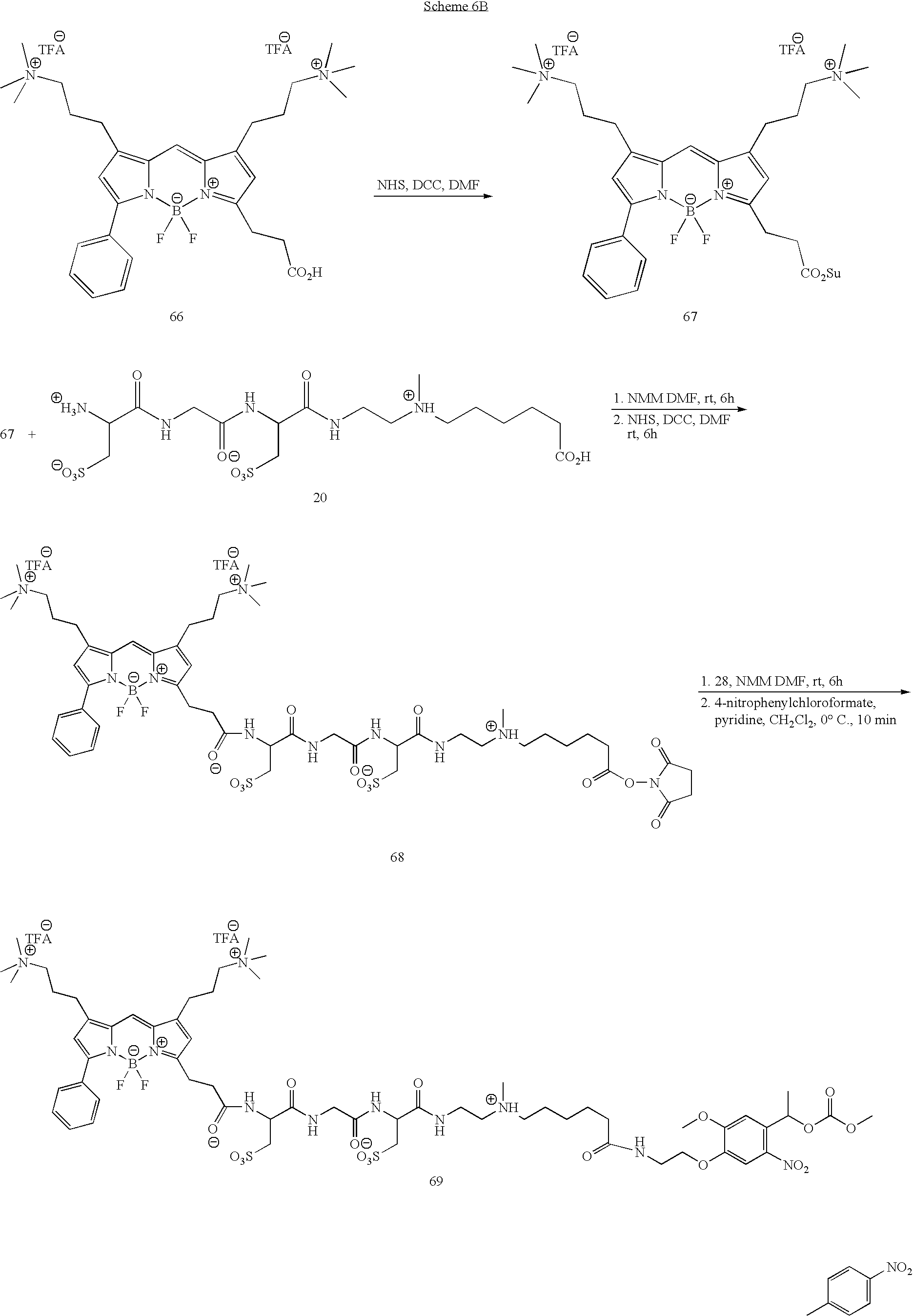

4,4-difluoro-1,7-(3,3-bistrimethylammonium trifluoroacetate)propyl-5-phenyl-4-bora-3a,4a,diaza-s-indacene-3-propionic acid, succinimidyl ester (67): A solution of 4,4-difluoro-1,7-(3,3-bistrimethylammonium trifluoroacetate)propyl-5-phenyl-4-bora-3a,4a,diaza-s-indacene-3-propionic acid (66) (6.6 mg, 0.00861 mmol), NHS (12 mg, 0.103 mmol) and DCC (21 mg, 0.103 mmol) in anhydrous DMF (0.3 mL) was stirred at room temperature under argon for 6 hours. The DMF was removed in vacuo at room temperature, and the residue purified by reverse phase HPLC to yield a red solid (4 mg, 0.00463 mmol, 54%): HRMS-ES (m/z): [M/2] calcd for C₃₄H₄₆BF₂N₅O₄²⁺ 318.6803. found 318.6799.

4,4-difluoro-1,7-(3,3-bistrimethylammonium)propyl-5-phenyl-4-bora-3a,4a,diaza-s-indacene-3-(6-(N-methyl-N-(2-(2-(2-(2-propionamido-3-sulfonate-propionamido)acetamido)-3-sulfonate-propionamido)ethyl)amino)hexanoic acid, succinimidyl ester, hydrotrifluoroacetate (68) A solution of 4,4-difluoro-1,7-(3,3-bistrimethylammonium trifluoroacetate)propyl-5-phenyl-4-bora-3a,4a,diaza-s-indacene-3-propionic acid, succinimidyl ester (67) (0.8 mg, 0.926 μmol), 6-(N-methyl-N-(2-(2-(2-(2-amino-3-sulfonate-propionamido)acetamido)-3-sulfonate-propionamido)ethyl)amino)hexanoic acid (20) (5 mg, 9.26 μmol) and NaHCO₃ (2.3 mg, 0.0278 mmol) in water (0.15 mL) was stirred at room temperature for 2 hours. The solvent was removed in vacuo and the resulting solid purified by reverse phase HPLC to yield a red solid (0.9 mg, 0.741 μmol, 80%): ¹H NMR (500 MHz, D₂O) δ 1.27 (m, 2H), 1.45-1.70 (m, 4H), 2.15 (m, 4H), 2.27 (q, 2H, J=7 Hz), 2.69-3.63 (m, 22H), 2.78 (s, 3H), 3.09 (s, 18H), 3.84 (m, 2H), 4.60-4.71 (m, 2H), 6.47 (s, 1H), 6.67 (s, 1H), 7.51-7.60 (m, 4H), 7.80-7.87 (m, 2H); HRMS-ES (m/z): [M] calcd for C₄₇H₇₁D₂BF₂N₉O₁₂S₂⁺ 1070.5001. found 1070.5027. The resulting red solid (0.9 mg, 0.762 μmol) was added to NHS (6 mg, 0.0534 mmol) and DCC (11 mg, 0.0534 mmol) in anhydrous DMF (0.2 mL) and stirred at room temperature under argon for 10 hours. The DMF was removed in vacuo at room temperature, and the residue purified by reverse phase HPLC to yield a red solid (1.0 mg, 0.762 μmol, 100%); ¹H NMR (500 MHz, D₂O) δ 1.32 (m, 2H), 1.55-1.70 (m, 4H), 2.09-2.22 (m, 4H), 2.59 (m, 2H), 2.70-3.65 (m, 25H), 2.76 (s, 4H), 3.07 (s, 9H), 3.08 (s, 9H), 3.81 (ddd, 4H, J1=J2=J3=16.4 Hz), 4.54-4.65 (m, 2H), 6.47 (s, 1H), 6.67 (s, 1H), 7.51 (m, 3H), 7.57 (s, 1H), 7.81 (m, 2H); HRMS-ES (m/z): [M] calcd for C₅₁H₇₆BF₂N₁₀O₁₄S₂⁺ 1165.5048. found 1165.5044.

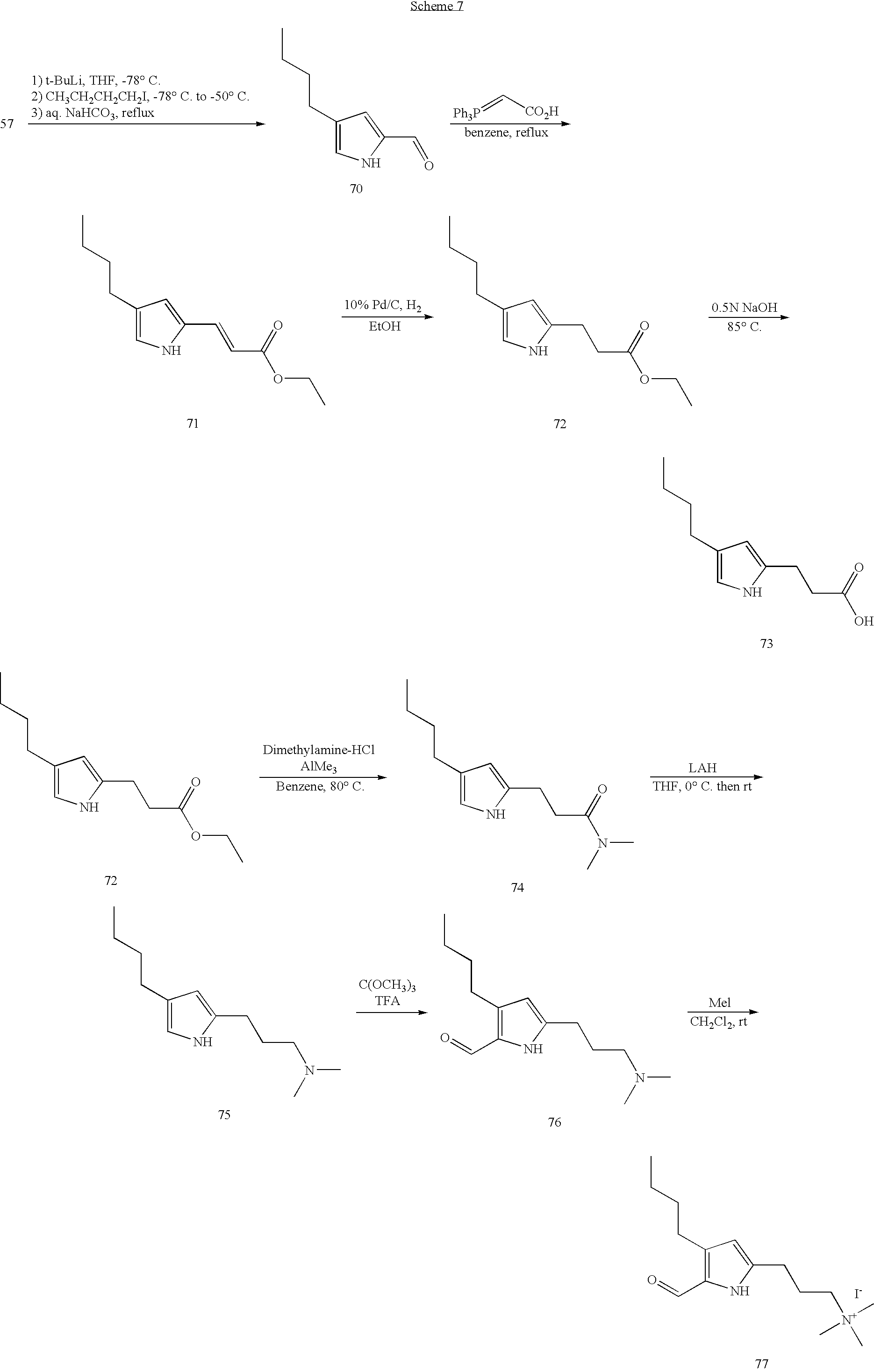

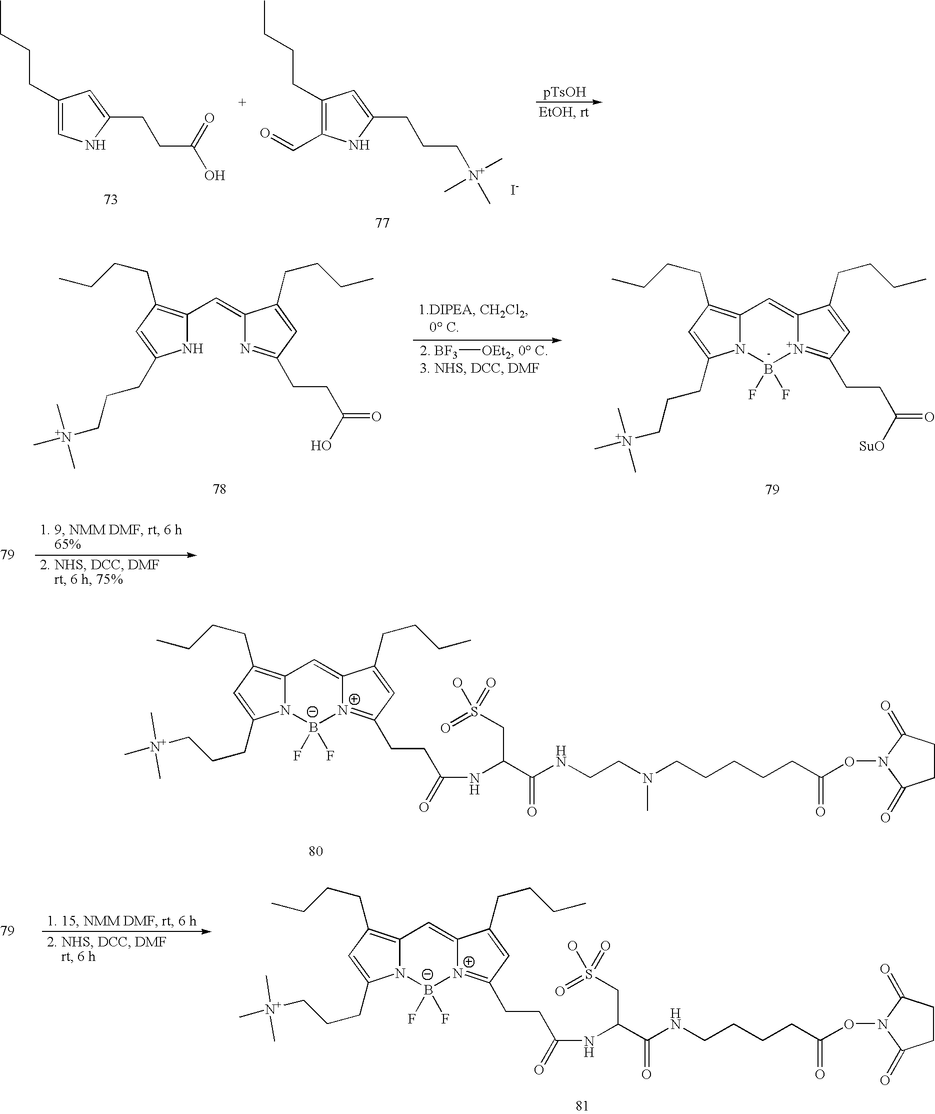

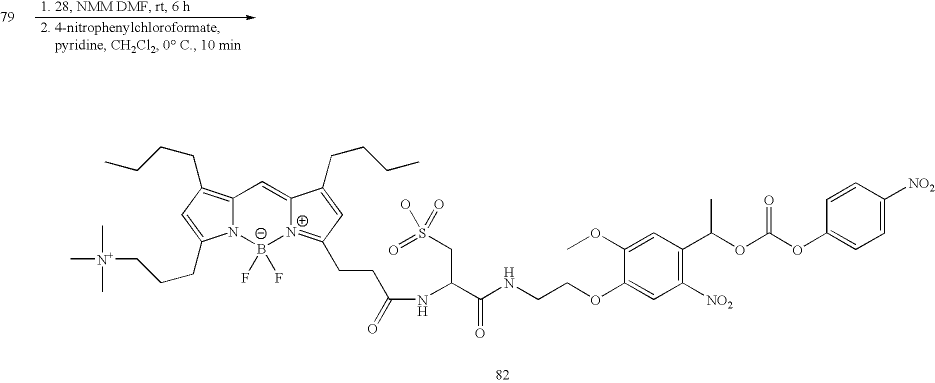

4-Butyl-1H-pyrrole-2-carbaldehyde (70): A 1.7 M solution of t-BuLi in pentane (5.85 mL, 9.95 mmol) was added dropwise to a solution of the 3-bromo-6-dimethylamino-1-azafulvene dimer (57) (1.0 g, 2.49 mmol) in anhydrous THF (115 mL) cooled to −78° C. under argon. The solution was maintained at −78° C. for 30 m, followed by addition of 1-iodobutane (1.14 mL, 9.97 mmol). The solution was allowed to warm to −50° C. over 1 h, followed by further stirring at ambient temperature for 30 min. Saturated NaHCO₃ (44 mL) and H₂O (44 mL) were added to the solution, and the mixture was refluxed 15 h. The mixture was allowed to cool to ambient temperature, and the product was isolated by extraction with DCM. The organic layer was dried (Na₂SO₄) and the solvent removed in vacuo providing a brown residue. The crude product was purified by flash chromatography on silica gel (9:1 hexanes-ethyl acetate) providing 70 as a brown oil (538 mg, 71%). ¹H-NMR (500 MHz, CDCl₃) δ 9.8 (bs, 1H), 9.4 (s, 1H), 6.9 (s, 1H), 6.75 (s, 1H), 2.45 (t, 2H), 1.5 (sextet, 2H), 1.3 (sextet, 2H), 0.99 (t, 3H).

(E)-Ethyl 3-(4-butyl-1H-pyrrol-2-yl)acrylate (71): A solution of aldehyde 70 (654 mg, 4.77 mmol) and (carbethoxymethylene)-triphenylphosphorane (2.49 g, 7.15 mmol) in anhydrous benzene (47 mL) was refluxed 15 h. The solution was allowed to cool to ambient temperature, and the solvent was removed in vacuo. The crude product was purified by flash chromatography on silica gel (9:1 hexanes-ethyl acetate) providing 71 as a clear yellow oil (980 mg, 99%). ¹H-NMR (500 MHz, CDCl₃) δ 8.4 (bs, 1H), 7.5 (d, 1H), 6.7 (s, 1H), 6.4 (s, 1H), 4.25 (q, 2H), 2.45 (t, 2H), 1.55 (sextet, 2H), 1.3-1.5 (m, 6H), 0.95 (t, 3H).

Ethyl 3-(4-butyl-1H-pyrrol-2-yl)propanoate (72). A flask containing a suspension of acrylate 71 (560 mg, 2.53 mmol) and 10% Pd/C (270 mg, 0.255 mmol) in ethanol (10 mL) was charged with hydrogen. The suspension was stirred under hydrogen (1 atm) for 2 h. The catalyst was filtered and rinsed with ethanol. The solvent was removed in vacuo providing 72 as a clear oil (468 mg, 2.10 mmol, 83%).

3-(5-Formyl-4-propyl-1H-pyrrol-2-yl)propanoic acid (73): A suspension of propanoate 72(568 mg, 2.10 mmol) in 0.5 M NaOH (50 mL) was stirred at 85° C. for 1 h. The mixture was cooled down to ambient temperature and acidified to pH 3 using 1 M HCl. The product was isolated by extraction with EtOAc. The organic layer was washed with brine (1×) and dried (Na₂SO₄). Removal of the solvent in vacuo provided 73 as a brown solid (409 mg, 2.10 mmol, 100%).

N,N-Dimethyl-3-(4-butyl-1H-pyrrol-2-yl)propanamide (74): To a flask containing a solution of dimethylamine hydrochloride (292 mg, 3.58 mmol) in anhydrous benzene (12.9 mL) under argon was added a solution of 2.0 M AlMe₃ in toluene (1.79 mL, 3.58 mmol). The mixture was stirred at ambient temperature for 1 h before addition of a solution of propanoate 72 (400 mg, 1.79 mmol) in anhydrous benzene (12.9 mL). The mixture was refluxed 15 h. The mixture was cooled to ambient temperature and quenched with 1 M HCl. Water (20 mL) was added, and the product was isolated by extraction with DCM. The organic layer was dried (Na₂SO₄) and the solvent removed in vacuo providing 74 as a white solid.

N,N-Dimethyl-3-(4-butyl-1H-pyrrol-2-yl)propan-1-amine (75): A solution of amide 74 (240 mg, 1.08 mmol) in anhydrous THF (27 mL) was slowly added to a stirred suspension of LAH (200 mg, 5.27 mmol) in anhydrous THF (39 mL) cooled to 0° C. under argon. The mixture was stirred at ambient temperature for 2 h. The mixture was cooled to 0° C., followed by quenching with 1.5 M Na₂CO₃. Water (30 mL) was added, and the product was isolated by extraction with EtOAc. The organic layer was washed with brine (1×) and dried (Na₂SO₄). Removal of the solvent in vacuo provided 75 as a brown oil.

### 3-(3-(dimethylamino)propyl)-5-methyl-1H-pyrrole-2-carbaldehyde (76)

Trimethyl orthoformate (0.350 mL, 3.20 mmol) was added to a flask containing a solution of pyrrole 75 (194 mg, 1.00 mmol) in TFA (3.5 mL) under argon at 0° C. The mixture was stirred at 0° C. for 1 h before being quenched with cold H₂O (5 mL). The mixture was basified using 1 M NaOH and the product was isolated by extraction with DCM. The organic layer was dried (Na₂SO₄) and the solvent removed in vacuo providing 76 as a yellow solid (151 mg, 0.78 mmol, 78%)

### 3-(3-(trimethylamonium iodide)propyl)-5-methyl-1H-pyrrole-2-carbaldehyde (77)

Iodomethane (1 mL) was added to a solution of amine 76 (31.1 mg, 0.160 mmol) in anhydrous DCM (3 mL). Upon addition of the iodomethane, a precipitate was noted. The suspension was stirred at ambient temperature for a further 40 min before the solvent was removed in vacuo providing 77 as a light brown solid (44.4 mg, 0.145 mmol, 91%)

5-(Trimethylammonium trifluoroacetate)-butyl-3-propyl-3′-butyl-5′-(3-propionic acid)dipyrromethene (78): Para-toluenesulfonic acid monohydrate (44.4 mg, 0.233 mmol) was added to a stirred suspension of ammonium iodide (77) (88.4 mg, 0.234 mmol) and acid (74) (45.6 mg, 0.234 mmol) in ethanol (3 mL) under normal atmosphere at ambient temperature. The mixture was stirred at ambient temperature for 30 min before removal of the solvent in vacuo. The crude product was purified via reverse phase HPLC using a gradient of 35:65 ([95% CH₃CN/4.9% H₂O/0.1% TFA]:[99.9% H₂O/0.1% TFA]) to 65:35 ([95% CH₃CN/4.9% H₂O/0.1% TFA]:[99.9% H₂O/0.1% TFA]) over 30 min at a flow rate of 20 mL/min, monitoring at 450 nm. The product was collected at 21 min. The solution containing the product was frozen, and the solvents removed via the use of a lyophilizer providing 78 as an orange solid (115 mg, 91%): ¹H NMR (500 MHz, CD₃OD) δ 0.98 (m, 6H), 1.71 (sext, J=7.5 Hz, 2H), 1.42 (sext, J=7.5 Hz, 4H), 1.49 (sext, J=7.5 Hz, 4H), 2.28 (m, 2H), 2.81 (m, 6H), 2.95 (t, J=7.5 Hz, 2H), 3.13 (t, J=7.5 Hz, 2H), 3.17 (s, 9H), 3.45 (m, 2H), 6.54 (s, 1H), 6.57 (s, 1H), 7.52 (s, 1H)

4,4-Difluoro-1-butyl-5-(trimethylammonium trifluoroacetate)-propyl-7-butyl-4-bora-3a,4a,diaza-s-indacene-3-propionic acid, succinimidyl ester (79). Freshly distilled N,N-diisopropylethylamine (400 μL) was added to a solution of dipyrromethene (78) (67 mg, 0.124 mmol) in anhydrous acetonitrile (3 mL) under argon at ambient temperature, and the resulting mixture was stirred at ambient temperature for 5 min. The mixture was cooled to 0° C. and BF₃.Et₂O (100 μL, 1.13 mmol) was added. The mixture was stirred at 0° C. for 30 min before the solvent was removed in vacuo at 0° C. The crude product was purified via reverse phase HPLC using a gradient of 35:65 ([95% CH₃CN/4.9% H₂O/0.1% TFA]:[99.9% H₂O/0.1% TFA]) to 55:45 ([95% CH₃CN/4.9% H₂O/0.1% TFA]:[99.9% H₂O/0.1% TFA]) over 30 min at a flow rate of 20 mL/min, monitoring at 450 nm. The product was collected at 21 min. The solution containing the product was frozen, and the solvents removed via the use of a lyophilizer providing an orange solid (17.0 mg, 23%): ¹H NMR (500 MHz, CD₃OD) δ 0.98 (m, 6H), 1.71 (sext, J=7.5 Hz, 2H), 1.42 (sext, J=7.5 Hz, 4H), 1.64 (sext, J=7.5 Hz, 4H), 2.25 (m, 2H), 2.70 (t, J=7.5 Hz, 2H), 2.99 (t, J=7.5 Hz, 2H), 3.16 (s, 9H), 3.18 (m, 2H), 3.41 (m, 2H), 6.28 (s, 1H), 6.34 (s, 1H), 7.48 (s, 1H)

The resulting orange solid was added to NHS (27 mg, 0.237 mmol) and DCC (34 mg, 0.165 mmol) in DMF (2 mL) under argon at ambient temperature. The mixture was stirred at ambient temperature for 6 h before the solvent was removed via the use of a lyophilizer. The crude product was purified via reverse phase HPLC using a gradient of 2:3 ([95% CH₃CN/4.9% H₂O/0.1% TFA]:[99.9% H₂O/0.1% TFA]) to 7:3 ([95% CH₃CN/4.9% H₂O/0.1% TFA]:[99.9% H₂O/0.1% TFA]) over 30 min at a flow rate of 20 mL/min, monitoring at 450 nm. The product was collected at 21.5 min. The solution containing the product was frozen, and the solvents removed via the use of a lyophilizer providing 79 as an orange solid (17.1 mg, 86%): ¹H NMR (500 MHz, CDCl₃) δ 0.94 (m, 6H), 1.37 (sext, J=7.5 Hz, 4H), 1.58 (sext, J=7.5 Hz, 4H), 1.94 (m, 2H), 2.67 (dt, J=8, 7.5 Hz, 4H), 2.77 (s, 4H), 2.94 (t, J=7.5 Hz, 2H), 3.03 (s, 9H), 3.06 (t, J=7.5 Hz, 2H), 3.24 (t, J=7.5 Hz, 2H), 3.32 (m, 2H), 6.33 (s, 1H), 6.36 (s, 1H), 7.49 (s, 1H); ¹³C NMR (125 MHz, CDCl₃) δ 14.25, 22.78, 23.25, 24.39, 25.95, 26.37, 26.40, 26.52, 30.40, 33.95, 54.06, 66.97, 117.84, 117.95, 124.08, 134.08, 134.29, 149.48, 150.03, 158.47, 159.93, 169.37, 171.13; HRMS [M] calcd for C₂₈H₄₀BF₂N₄O₄⁺ 573.3419. found 573.3417.

6-(4,4-Difluoro-1-propyl-5-(trimethylammonium trifluoroacetate)-propyl-7-propyl-4-bora-3a,4a,diaza-s-indacene-3-(N-methyl-N-(2-((R)-2-propionamido-3-sulfono-propionamido)ethyl)amino)hexanoic acid, succinimidyl ester (80): N-methylmorpholine (54 μL) was added to a stirred mixture of succinimidyl ester (79) (17.1 mg, 0.0249 mmol)) and hexanoic acid 9 (30 mg, 0.0748 mmol) in anhydrous DMF (1.5 mL) under argon at ambient temperature. The mixture was stirred at ambient temperature for 4 h before the solvent was removed via the use of a lyophilizer. The crude product was purified via reverse phase HPLC using a gradient of 3:7 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 450 nm. The solution containing the product was frozen, and the solvents removed via the use of a lyophilizer providing an orange solid (16.5 mg, 73%).

The resulting orange solid was added to NHS (16 mg, 0.0139 mmol) and DCC (26 mg, 0.0126 mmol) in DMF (1.8 mL) under argon at ambient temperature. The mixture was stirred at ambient temperature for 7 h before the solvent was removed via the use of a lyophilizer. The crude product was purified via reverse phase HPLC using a gradient of 3:7 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 450 nm. The product was collected at 20.5 min. The solution containing the product was frozen, and the solvents removed via the use of a lyophilizer providing 80 as an orange solid (12.4 mg, 77%): ¹H NMR (500 MHz, CD₃OD) δ 0.94 (m, 6H), 1.37 (m, 6H), 1.44 (m, 4H), 1.58 (m, 4H), 1.94 (m, 2H), 2.24 (m, 2H), 2.66 (m, 8H), 2.77 (m, 9H), 2.93 (m, 5H), 3.03 (s, 9H), 3.14 (m, 4H), 3.30 (m, 4H), 4.68 (m, 1H), 6.29 (s, 1H), 6.32 (s, 1H), 7.45 (s, 1H); ¹³C NMR (125 MHz, CD₃CN) δ 14.25, 22.98, 23.24, 23.88, 24.81, 25.30, 25.92, 26.22, 26.34, 26.50, 31.20, 33.92, 34.00, 34.68, 34.87, 35.36, 41.11, 41.36, 51.77, 51.82, 52.68, 52.84, 54.03, 54.06, 56.44, 56.53, 56.91, 57.24, 66.94, 117.54, 123.62, 133.78, 134.23, 149.09, 149.82, 158.79, 161.40, 170.16, 171.30, 172.43, 172.60, 172.84; HRMS [M] calcd for C₄₀H₆₃BF₂N₇O₉S⁺ 894.4778. found 894.4769.

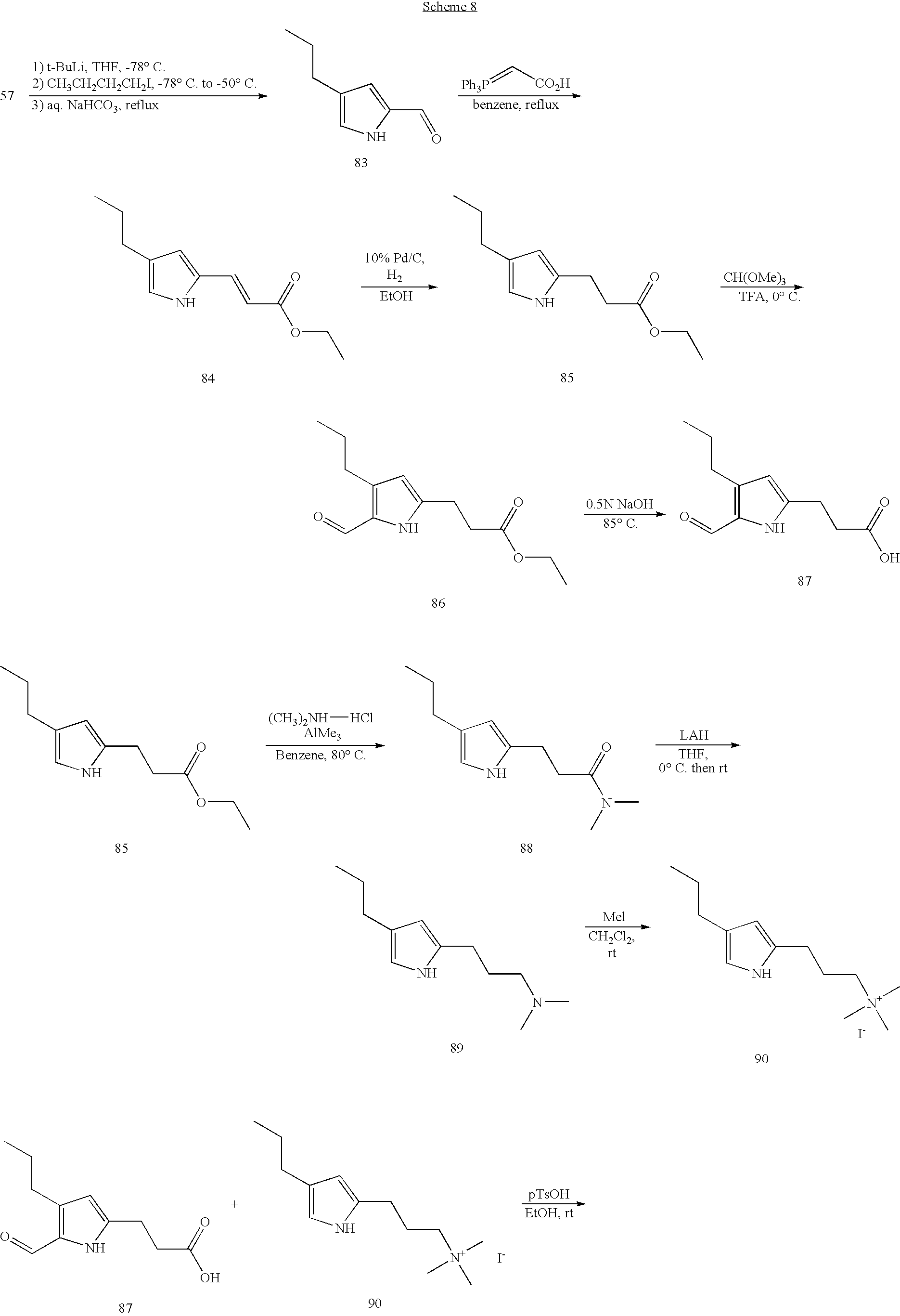

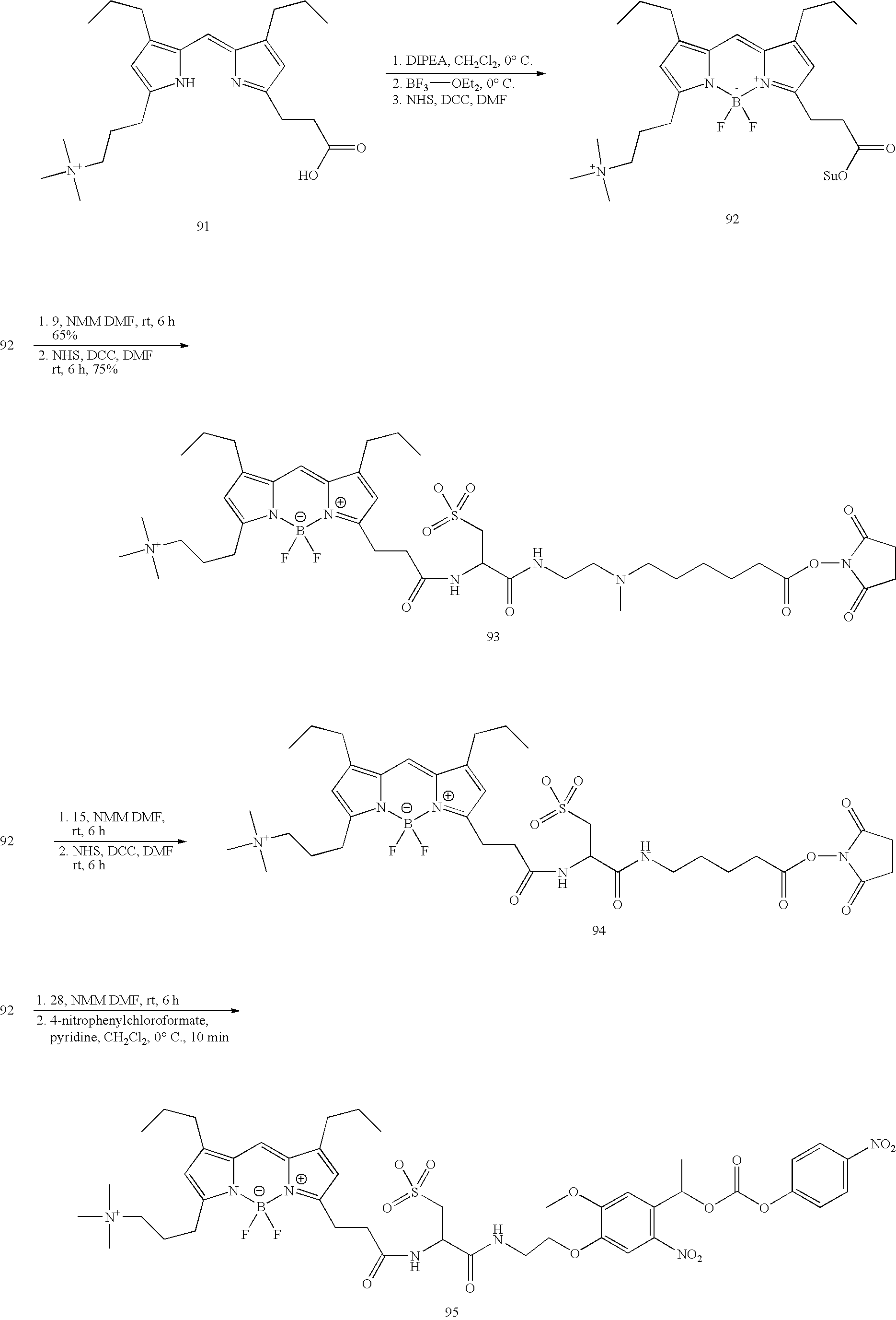

4-Propyl-1H-pyrrole-2-carbaldehyde (83): A 1.7 M solution of t-BuLi in pentane (11.7 mL, 19.9 mmol) was added dropwise to a solution of the 3-bromo-6-dimethylamino-1-azafulvene dimer (57) (2.00 g, 4.97 mmol) in anhydrous THF (230 mL) cooled to −78° C. under argon. The solution was maintained at −78° C. for 30 m, followed by addition of 1-iodopropane (1.95 mL, 20.0 mmol). The solution was allowed to warm to −50° C. over 1 h, followed by further stirring at ambient temperature for 30 min. Saturated NaHCO₃ (88 mL) and H₂O (88 mL) were added to the solution, and the mixture was refluxed 15 h. The mixture was allowed to cool to ambient temperature, and the product was isolated by extraction with DCM. The organic layer was dried (Na₂SO₄) and the solvent removed in vacuo providing a brown residue. The crude product was purified by flash chromatography on silica gel (9:1 hexanes-ethyl acetate) providing 83 as a brown oil (902 mg, 6.58 mmol, 66%): R_{f} 0.35 (4:1 hexanes-ethyl acetate); FTIR (CH₂Cl₂): 776 (s), 833 (m), 969 (m), 1131 (s), 1151 (m), 1353 (s), 1398 (s), 1446 (s), 1489 (m), 1645 (s), 2871 (m), 2930 (s), 2959 (s), 3272 (br); ¹H NMR (500 MHz, CDCl₃) δ 0.96 (t, J=7.5 Hz, 3H), 1.61 (sext, J=7.5 Hz, 2H), 2.47 (t, J=7.5 Hz, 2H), 6.83 (s, 1H), 6.97 (s, 1H), 9.43 (s, 1H), 10.12 (bs, 1H) (NH); ¹³C NMR (125 MHz, CDCl₃) δ 13.92, 24.16, 28.76, 121.85, 125.89, 127.64, 132.64, 179.35; HRMS-EI (m/z): [M] calcd for C₈H₁₂NO 138.0914. found 138.0909.

(E)-Ethyl 3-(4-propyl-1H-pyrrol-2-yl)acrylate (84): A solution of aldehyde 83 (654 mg, 4.77 mmol) and (carbethoxymethylene)-triphenylphosphorane (2.49 g, 7.15 mmol) in anhydrous benzene (47 mL) was refluxed 15 h. The solution was allowed to cool to ambient temperature, and the solvent was removed in vacuo. The crude product was purified by flash chromatography on silica gel (9:1 hexanes-ethyl acetate) providing 84 as a light brown oil (980 mg, 4.73 mmol, 99%): R_{f} 0.42 (4:1 hexanes-ethyl acetate); FTIR (CH₂Cl₂): 1181 (s), 1279 (s), 1365 (m), 1404 (m), 1439 (m), 1569 (s), 1621 (s), 1677 (s), 2869 (m), 2929 (m), 2953 (m), 3302 (br); ¹H NMR (500 MHz, CDCl₃) δ 0.96 (t, J=7.5 Hz, 3H), 1.33 (t, J=7.5 Hz, 3H), 1.60 (sext, J=7.5 Hz, 2H), 2.45 (t, J=7.5 Hz, 2H), 4.26 (q, J=7.5 Hz, 2H), 6.07 (d, J=16 Hz, 1H), 6.43 (s, 1H), 6.72 (s, 1H), 7.56 (d, J=16 Hz, 1H), 9.16 (bs, 1H) (NH); ¹³C NMR (125 MHz, CDCl₃) δ 14.04, 14.48, 24.24, 29.01, 60.40, 110.47, 114.46, 120.59, 127.20, 128.40, 134.92, 168.46; HRMS-EI (m/z): [M+H] calcd for C₁₂H₁₈NO₂ 208.1332. found 208.1335.

Ethyl 3-(4-propyl-1H-pyrrol-2-yl)propanoate (85). A flask containing a suspension of acrylate 84 (900 mg, 4.34 mmol) and 10% Pd/C (460 mg, 0.432 mmol) in ethanol (12 mL) was charged with hydrogen. The suspension was stirred under hydrogen (1 atm) for 2 h. The catalyst was filtered and rinsed with ethanol. The solvent was removed in vacuo providing 85 as a clear oil (754 mg, 3.60 mmol, 83%): R_{f} 0.27 (9:1 hexanes-ethyl acetate); FTIR (CH₂Cl₂): 794 (m), 1039 (m), 1113 (m), 1194 (s), 1374 (s), 1445 (m), 1723 (m), 2871 (s), 2928 (s), 2957 (s), 3388 (br); ¹H NMR (500 MHz, CDCl₃) δ 0.99 (t, J=7.5 Hz, 3H), 1.30 (t, J=7 Hz, 3H), 1.61 (sext, J=7.5 Hz, 2H), 2.44 (t, J=7.5 Hz, 2H), 2.65 (t, J=7 Hz, 2H), 2.90 (t, J=7 Hz, 2H), 4.19 (q, J=7 Hz, 2H), 5.82 (s, 1H), 6.46 (s, 1H), 8.30 (bs, 1H) (NH); ¹³C NMR (125 MHz, CDCl₃) δ 14.28, 14.32, 22.90, 24.40, 29.41, 34.70, 60.75, 106.12, 113.87, 124.49, 130.99, 174.17; HRMS-EI (m/z): [M+H] calcd for C₁₂H₂₀NO₂ 210.1489. found 210.1489.

Ethyl 3-(5-formyl-4-propyl-1H-pyrrol-2-yl)propanoate (86): Trimethyl orthoformate (0.35 mL, 3.20 mmol) was added to a solution of pyrrole 85 (209 mg, 1.00 mmol) in TFA (3.5 mL) cooled to 0° C. under argon. The solution was stirred at 0° C. for 1 h before H₂O (2 mL) was added. The solution was basified to pH 12 using 1 M NaOH, and the product was isolated by extraction with DCM. The organic layer was dried (Na₂SO₄) and the solvent removed in vacuo providing 86 as a yellow oil (213 mg, 0.90 mmol, 90%): R_{f} 0.30 (3:1 hexanes-ethyl acetate); FTIR (CH₂Cl₂): 812 (m), 1162 (s), 1185 (s), 1353 (s), 1373 (s), 1475 (s), 1629 (s), 1735 (s), 2871 (m), 2932 (s), 2960 (s), 3251 (br); ¹H NMR (500 MHz, CDCl₃) δ 0.94 (t, J=7.5 Hz, 3H), 1.22 (t, J=7.5 Hz, 3H), 1.62 (sext, J=7.5 Hz, 2H), 2.66 (dt, J=7.5, 1.5 Hz, 4H), 2.96 (t, J=7.5 Hz, 2H), 4.13 (q, J=7 Hz, 2H), 5.90 (s, 1H), 9.47 (s, 1H), 10.52 (bs, 1H), (NH); ¹³C NMR (125 MHz, CDCl₃) δ 13.96, 14.27, 23.10, 24.89, 27.48, 33.64, 60.80, 110.06, 128.02, 139.54, 141.09, 172.83, 176.59; HRMS-EI (m/z): [M+H] calcd for C₁₃H₂₀NO₃ 238.1438. found 238.1436.

3-(5-Formyl-4-propyl-1H-pyrrol-2-yl)propanoic acid (87): A suspension of propanoate 86 (125 mg, 0.527 mmol) in 0.5 M NaOH (10 mL) was stirred at 85° C. for 1 h. The mixture was cooled down to ambient temperature and acidified to pH 3 using 1 M HCl. The product was isolated by extraction with EtOAc. The organic layer was washed with brine (1×) and dried (Na₂SO₄). Removal of the solvent in vacuo provided 87 as a brown solid (106 mg, 0.507 mmol, 96%): FTIR (CH₂Cl₂): ¹H NMR (500 MHz, CD₃OD) δ 0.95 (t, J=7.5 Hz, 3H), 1.63 (sext, J=7.5 Hz, 2H), 2.64 (t, J=7.5 Hz, 2H), 2.69 (t, J=7.5 Hz, 2H), 2.89 (t, J=7.5 Hz, 2H), 5.97 (s, 1H), 9.40 (s, 1H); ¹³C NMR (125 MHz, CDCl₃) δ HRMS-EI (m/z): [M+H] calcd for C₁₁H₁₆NO₃ 210.1124. found 210.1123.

N,N-Dimethyl-3-(4-propyl-1H-pyrrol-2-yl)propanamide (88): To a flask containing a solution of dimethylamine hydrochloride (163 mg, 2.00 mmol) in anhydrous benzene (7.2 mL) under argon was added a solution of 2.0 M AlMe₃ in toluene (1.00 mL, 2.00 mmol). The mixture was stirred at ambient temperature for 1 h before addition of a solution of propanoate 85 (210 mg, 1.00 mmol) in anhydrous benzene (7.2 mL). The mixture was refluxed 15 h. The mixture was cooled to ambient temperature and quenched with 1 M HCl. Water (20 mL) was added, and the product was isolated by extraction with DCM. The organic layer was dried (Na₂SO₄) and the solvent removed in vacuo providing 88 as a white solid (191 mg, 0.918 mmol, 92%): mp 71-72° C.; HRMS-EI (m/z): FTIR (CH₂Cl₂): ¹H NMR (500 MHz, CDCl₃) δ 1.00 (t, J=7.5 Hz, 3H), 1.62 (sext, J=7.5 Hz, 2H), 2.45 (t, J=7.5, 2H), 2.64 (t, J=6.5 Hz, 2H), 2.93 (t, J=6.5 Hz, 2H), 2.98 (s, 3H), 3.00 (s, 3H), 5.80 (s, 1H), 6.45 (s, 1H), 9.08 (bs, 1H) (NH); ¹³C NMR (125 MHz, CDCl₃) δ 14.12, 22.68, 24.26, 29.26, 34.06, 35.42, 36.94, 105.46, 113.49, 123.72, 131.72, 173.04; [M+H] calcd for C₁₂H₂₁N₂O 209.1648. found 209.1645.

N,N-Dimethyl-3-(4-propyl-1H-pyrrol-2-yl)propan-1-amine (89): A solution of amide 88 (160 mg, 0.769 mmol) in anhydrous THF (28 mL) was slowly added to a stirred suspension of LAH (150 mg, 3.95 mmol) in anhydrous THF (19.5 mL) cooled to 0° C. under argon. The mixture was stirred at ambient temperature for 2 h. The mixture was cooled to 0° C., followed by quenching with 1.5 M Na₂CO₃. Water (30 mL) was added, and the product was isolated by extraction with EtOAc. The organic layer was washed with brine (1×) and dried (Na₂SO₄). Removal of the solvent in vacuo provided 89 as a brown oil (146 mg, 0.752 mmol, 98%): FTIR (CH₂Cl₂): 791 (m), 1464 (s), 1687 (s), 2779 (s), 2859 (s), 2928 (s), 2953 (s), 3256 (br); ¹H NMR (500 MHz, CDCl₃) δ 0.98 (t, J=7.5 Hz, 3H), 1.60 (sext, J=7.5 Hz, 2H), 1.80 (p, J=7 Hz, 2H), 2.26 (s, 6H), 2.35 (t, J=7 Hz, 2H), 2.44 (t, J=7.5 Hz, 2H), 2.64 (t, J=7 Hz, 2H), 5.79 (s, 1H), 6.44 (s, 1H), 8.78 (bs, 1H) (NH); ¹³C NMR (125 MHz, CDCl₃) δ 14.37, 24.47, 26.21, 27.39, 29.54, 45.54, 59.66, 105.56, 113.32, 124.60, 132.25; HRMS-EI (m/z): [M] calcd for C₁₂H₂₃N₂ 195.1856. found 195.1852.

Trimethyl (3-(4-propyl-1H-pyrrol-2-yl)propyl)ammonium iodide (90): Iodomethane (1 mL) was added to a solution of amine 89 (55.5 mg, 0.286 mmol) in anhydrous DCM (1 mL) under argon. The mixture was stirred at ambient temperature for 1 h before the solvents were removed in vacuo providing 77 as a light brown oil (96 mg, 0.285 mmol, 100%): FTIR (CH₂Cl₂): ¹H NMR (500 MHz, CD₃OD δ 0.92 (t, J=7.5 Hz, 3H), 1.54 (sext, J=7.5 Hz, 2H), 2.09 (m, 2H), 2.36 (t, J=7.5 Hz, 2H), 2.67 (t, J=7.5 Hz, 2H), 3.14 (s, 9H), 3.38 (m, 2H), 5.77 (s, 1H), 6.40 (s, 1H), 9.70 (bs, 1H) (NH); ¹³C NMR (125 MHz, CD₃OD) δ 14.51, 24.65, 25.40, 25.62, 30.52, 54.04, 67.62, 107.16, 107.19, 115.07, 115.24, 125.01, 130.64, 130.81; HRMS-EI (m/z): [M] calcd for C₁₃H₂₅N₂ 209.2012. found 209.2015.

5-(Trimethylammonium trifluoroacetate)-propyl-3-propyl-3′-propyl-5′-(3-propionic acid)dipyrromethene (91): Para-toluenesulfonic acid monohydrate (8.8 mg, 0.0463 mmol) was added to a stirred suspension of ammonium iodide (90) (15.5 mg, 0.0461 mmol) and acid (87) (8.4 mg, 0.0463 mmol) in ethanol (3 mL) under normal atmosphere at ambient temperature. The mixture was stirred at ambient temperature for 30 min before removal of the solvent in vacuo. The crude product was purified via reverse phase HPLC using a gradient of 3:7 ([95% CH₃CN/4.9% H₂O/0.1% TFA]:[99.9% H₂O/0.1% TFA]) to 1:1 ([95% CH₃CN/4.9% H₂O/0.1% TFA]:[99.9% H₂O/0.1% TFA]) over 30 min at a flow rate of 20 mL/min, monitoring at 450 nm. The product was collected at 10 min. The solution containing the product was frozen, and the solvents removed via the use of a lyophilizer providing 85 as an orange solid (19.7 mg, 0.0384 mmol, 83%): FTIR (CH₂Cl₂): ¹H NMR (500 MHz, CDCl₃) δ 1.01 (t, J=7.5 Hz, 3H), 1.02 (t, J=7.5 Hz, 3H), 1.71 (sext, J=7.5 Hz, 2H), 1.72 (sext, J=7.5 Hz, 2H), 2.28 (m, 2H), 2.81 (m, 6H), 2.95 (t; J=7.5 Hz, 2H), 3.14 (t, J=7.5 Hz, 2H), 3.17 (s, 9H), 3.44 (m, 2H), 6.53 (s, 1H), 6.57 (s, 1H), 7.51 (s, 1H); ¹³C NMR (125 MHz, CDCl₃) δ 14.21, 14.26, 23.34, 25.10, 25.44, 25.50, 26.25, 29.49, 33.14, 53.87, 67.00, 116.89, 117.31, 123.68, 129.07, 129.42, 154.14, 154.95, 157.60, 160.56, 175.40; HRMS [M] calcd for C₂₄H₃₈N₃O₂⁺ 400.2958. found 400.2957.

4,4-Difluoro-1-propyl-5-(trimethylammonium trifluoroacetate)-propyl-7-propyl-4-bora-3a,4a,diaza-s-indacene-3-propionic acid, succinimidyl ester (92). Freshly distilled N,N-diisopropylethylamine (0.100 mL, 0.574 mmol) was added to a solution of dipyrromethene (91) (15.4 mg, 0.0300 mmol) in anhydrous acetonitrile (3.0 mL) under argon at ambient temperature, and the resulting mixture was stirred at ambient temperature for 5 min. The mixture was cooled to 0° C. and BF₃.Et₂O (25.0 μL, 0.199 mmol) was added. The mixture was stirred at 0° C. for 30 min before the solvent was removed in vacuo at 0° C. The crude product was purified via reverse phase HPLC using a gradient of 35:65 ([95% CH₃CN/4.9% H₂O/0.1% TFA]:[99.9% H₂O/0.1% TFA]) to 55:45 ([95% CH₃CN/4.9% H₂O/0.1% TFA]:[99.9% H₂O/0.1% TFA]) over 30 min at a flow rate of 20 mL/min, monitoring at 450 nm. The product was collected at 18 min. The solution containing the product was frozen, and the solvents removed via the use of a lyophilizer providing an orange solid (5.5 mg, 0.00979 mmol, 33%): ¹H NMR (500 MHz, CDCl₃) δ 0.99 (t, J=7.5 Hz, 3H), 1.01 (t, J=7 Hz, 3H), 1.66 (sext, J=7.5 Hz, 2H), 1.68 (sext, J=7 Hz, 2H), 2.24 (m, 2H), 2.69 (m, 6H), 3.00 (t, J=7.5 Hz, 2H), 3.14 (s, 9H), 3.18 (t, J=7.5 Hz, 2H), 3.41 (m, 2H), 6.29 (s, 1H), 6.34 (s, 1H), 7.50 (s, 1H); ¹³C NMR (125 MHz, CDCl₃) δ 14.27, 14.33, 23.75, 25.28, 25.42, 25.49, 26.39, 28.89, 28.92, 33.85, 53.72, 67.46, 117.87, 118.27, 123.60, 134.50, 135.00, 149.05, 149.91, 158.89, 161.59, 176.06; HRMS [M] calcd for C₂₄H₃₇BF₂N₃O₂⁺ 448.2943. found 448.2938.

The resulting orange solid was added to NHS (35.5 mg, 0.308 mmol) and DCC (52.0 mg, 0.252 mmol) under argon at ambient temperature. The mixture was stirred at ambient temperature for 6 h before the solvent was removed via the use of a lyophilizer. The crude product was purified via reverse phase HPLC using a gradient of 2:3 ([95% CH₃CN/4.9% H₂O/0.1% TFA]:[99.9% H₂O/0.1% TFA]) to 7:3 ([95% CH₃CN/4.9% H₂O/0.1% TFA]:[99.9% H₂O/0.1% TFA]) over 30 min at a flow rate of 20 mL/min, monitoring at 450 nm. The product was collected at 18 min. The solution containing the product was frozen, and the solvents removed via the use of a lyophilizer providing 92 as an orange solid (21.6 mg, 0.0328 mmol, 85%): FTIR (CH₂Cl₂): ¹H NMR (500 MHz, CDCl₃) δ 0.97 (t, J=7.5 Hz, 3H), 0.98 (t, J=7.5 Hz, 3H), 1.63 (sext, J=7.5 Hz, 2H), 1.65 (sext, J=7.5 Hz, 2H), 2.17 (m, 2H), 2.67 (dt, J=8, 7.5 Hz, 4H), 2.78 (s, 4H), 2.95 (t, J=7.5 Hz, 2H), 3.03 (s, 9H), 3.06 (t, J=7.5 Hz, 2H), 3.25 (t, J=7.5 Hz, 2H), 3.31 (m, 2H), 6.34 (s, 1H), 6.36 (s, 1H), 7.50 (s, 1H); ¹³C NMR (125 MHz, CDCl₃) δ 14.54, 23.08, 24.68, 25.36, 25.41, 26.25, 26.81, 28.92, 28.94, 30.69, 67.27, 118.21, 118.29, 124.50, 134.48, 134.66, 149.60, 150.12, 158.79, 160.19, 169.66, 171.42; HRMS [M] calcd for C₂₈H₄₀BF₂N₄O₄⁺ 545.3110. found 545.3135.

6-(4,4-Difluoro-1-propyl-5-(trimethylammonium trifluoroacetate)-propyl-7-propyl-4-bora-3a,4a,diaza-s-indacene-3-(N-methyl-N-(2-((R)-2-propionamido-3-sulfono-propionamido)ethyl)amino)hexanoic acid, succinimidyl ester (93): N-methylmorpholine (70.0 μL, 0.651 mmol) was added to a stirred mixture of succinimidyl ester (92) (21.6 mg, 0.0328 mmol) and hexanoic acid 9 (41.0 mg, 0.102 mmol) in anhydrous DMF (3 mL) under argon at ambient temperature. The mixture was stirred at ambient temperature for 4 h before the solvent was removed via the use of a lyophilizer. The crude product was purified via reverse phase HPLC using a gradient of 3:7 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 450 nm. The product was collected at 14 min. The solution containing the product was frozen, and the solvents removed via the use of a lyophilizer providing an orange solid (21.1 mg, 0.0239 mmol, 73%): FTIR (CH₂Cl₂): ¹H NMR (500 MHz, CDCl₃) δ 0.99 (t, J=7.5 Hz, 3H), 1.00 (t, J=7.5 Hz, 3H), 1.43 (m, 2H), 1.65-1.70 (m, 6H), 1.78 (m, 2H), 2.24 (m, 2H), 2.33 (m, 2H), 2.69 (m, 6H), 2.88 (s, 3H), 3.00 (t, J=7.5 Hz, 2H), 3.05 (m, 1H), 3.15 (s, 914), 3.18-3.28 (m, 4H), 3.30-3.55 (m, 4H), 3.43 (m, 2H), 3.60-3.64 (m, 1H), 4.68 (m, 1H), 6.29 (s, 1H), 6.34 (s, 1H), 7.50 (s, 1H), 8.22 (m, 1H) (NH), 8.44 (m, 1H) (NH); ¹³C NMR (125 MHz, CDCl₃) δ 14.33, 23.74, 24.76, 25.42, 25.48, 25.54, 25.72, 26.42, 27.16, 28.91, 34.68, 35.44, 35.82, 40.95, 52.23, 53.16, 53.29, 53.78, 57.29, 57.80, 67.41, 117.93, 118.38, 123.62, 134.55, 134.97, 149.18, 149.90, 159.08, 161.34, 173.69, 174.34, 177.31; HRMS [M] calcd for C₃₆H₆₀BF₂N₆O₇S⁺ 769.4301. found 769.4243. The resulting orange solid was added to NHS (8.0 mg, 0.0695 mmol) and DCC (11.0 mg, 0.0533 mmol) under argon at ambient temperature. The mixture was stirred at ambient temperature for 7 h before the solvent was removed via the use of a lyophilizer. The crude product was purified via reverse phase HPLC using a gradient of 3:7 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 450 nm. The product was collected at 17 min. The solution containing the product was frozen, and the solvents removed via the use of a lyophilizer providing 93 as an orange solid (23.4 mg, 0.0239 mmol, 100%): ¹H NMR (500 MHz, CD₃OD) δ 1.00 (t, J=7.5 Hz, 3H), 1.00 (t, J=7 Hz, 3H), 1.52 (m, 2H), 1.68 (m, 4H), 1.81 (m, 4H), 2.24 (m, 2H), 2.69 (m, 8H), 2.82 (m, 4H), 2.89 (m, 3H), 3.00 (t, J=7.5 Hz, 2H), 3.06 (m, 1H), 3.15 (s, 9H), 3.20 (m, 4H), 3.41 (m, 4H), 3.62-3.75 (m, 1H), 4.68 (m, 1H), 6.30 (s, 1H), 6.35 (s, 1H), 7.50 (s, 1H); ¹³C NMR (125 MHz, CD₃CN) δ 14.27, 22.97, 23.85, 24.80, 25.07, 25.14, 25.92, 26.22, 26.50, 28.59, 31.20, 34.79, 34.95, 35.22, 41.04, 41.25, 51.77, 51.81, 52.71, 52.85, 53.99, 54.02, 56.54, 56.80, 57.18, 66.90, 117.60, 118.26, 123.68, 133.84, 134.30, 148.84, 149.61, 158.80, 161.49, 170.18, 171.36, 172.55, 172.65, 172.82; HRMS [M] calcd for C₄₀H₆₃BF₂N₇O₉S⁺ 866.4465. found 866.4456.

(94): To a stirred solution of NHS ester 92 (45 mg, 0.068 mmol) and 15 (50 mg, 0.186 mmol) in DMF (6 mL) at room temperature was added 4-methylmorpholine (0.16 mL, 1.42 mmol). After 4 h, the solvent was removed by vacuum and the crude mixture was purified by HPLC (30% B to 60% B over 30 min, 20 mL/min flow, λ=450 nm, product Rt=˜12 min) to provide an orange solid (20 mg, 43%) ¹H NMR (CDCl₃, 500 MHz): δ 7.47 (s, 1H), 6.30 (brs, 2H), 4.63 (dd, J=8.0 Hz, J=5.0 Hz, 1H), 3.40 (m, 2H), 3.18 (m, 6H), 3.12 (s, 9H), 2.96 (t, J=7.5 Hz, 2H), 2.66 (m, 6H), 2.28 (t, J=7.5 Hz, 2H), 2.21 (m, 2H), 1.63 (m, 4H), 1.59 (m, 2H), 1.51 (m, 2H), 0.97 (m, 6H). The resulting orange solid (20 mg, 0.029 mmol) was added to an argon flushed flask followed by DCC (136 mg, 0.66 mmol) and NHS (94 mg, 0.822 mmol). Then DMF (5.5 mL) was added and stirred at room temperature for 12 h. Solvent was removed under vacuum and the crude solid mass was dissolved/suspended in 3:1 H₂O:acetonitrile, filtered through a syringe filter, and purified by RP HPLC (30% B to 60% B over 30 min, 20 mL/min flow, λ=450 nm, product Rt=16 min) to afford the pure product as an orange solid (21.6 mg, 93%) ¹H NMR (CDCl₃, 500 MHz): δ 7.48 (s, 1H), 6.32 (s, 1H), 6.30 (s, 1H), 4.62 (dd, J=8.0 Hz, J=5.5 Hz, 1H), 3.40 (m, 2H), 3.18 (m, 6H), 3.12 (s, 9H), 2.96 (t, J=7.5 Hz, 2H), 2.78 (s, 4H), 2.65 (m, 8H), 2.22 (m, 2H), 1.65 (m, 8H), 0.98 (m, 6H).

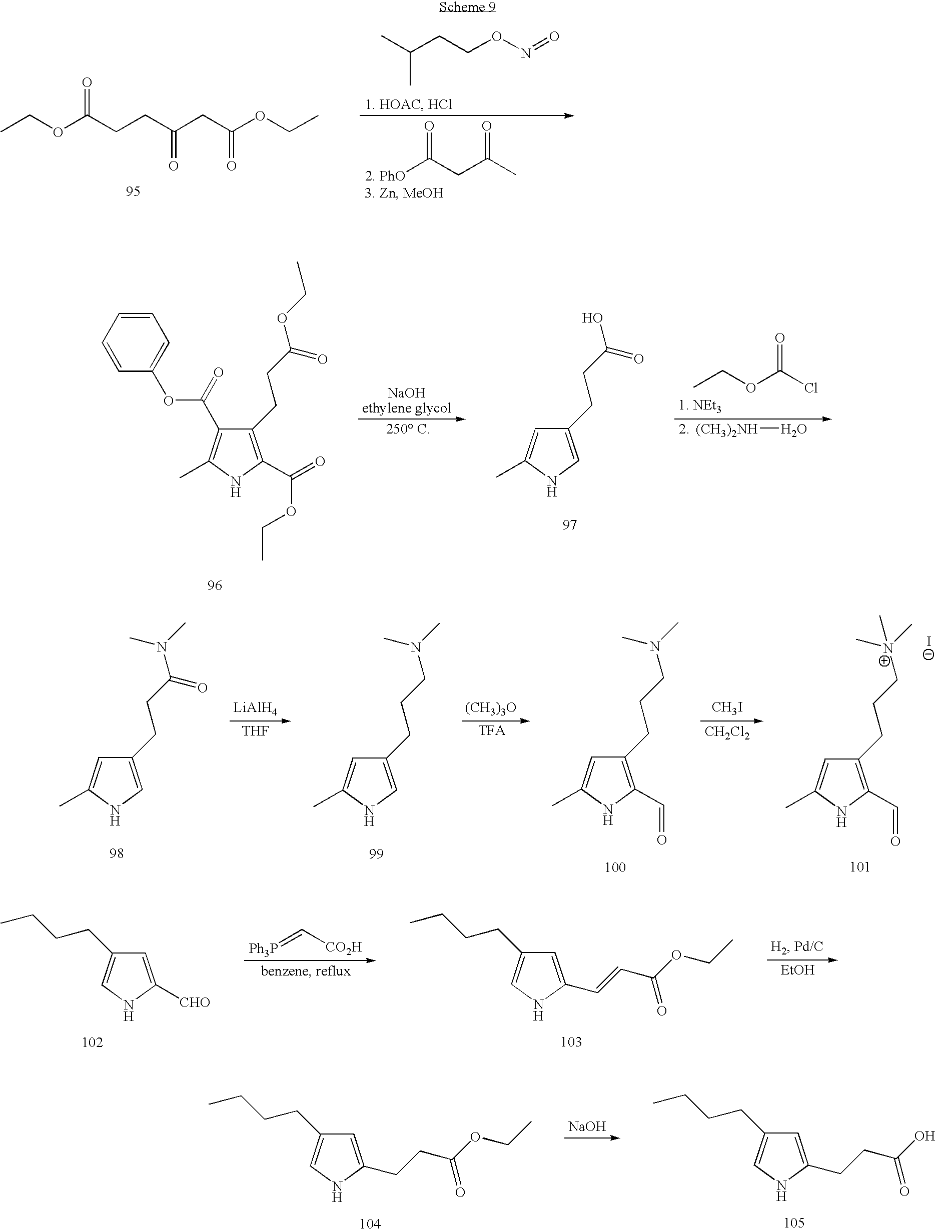

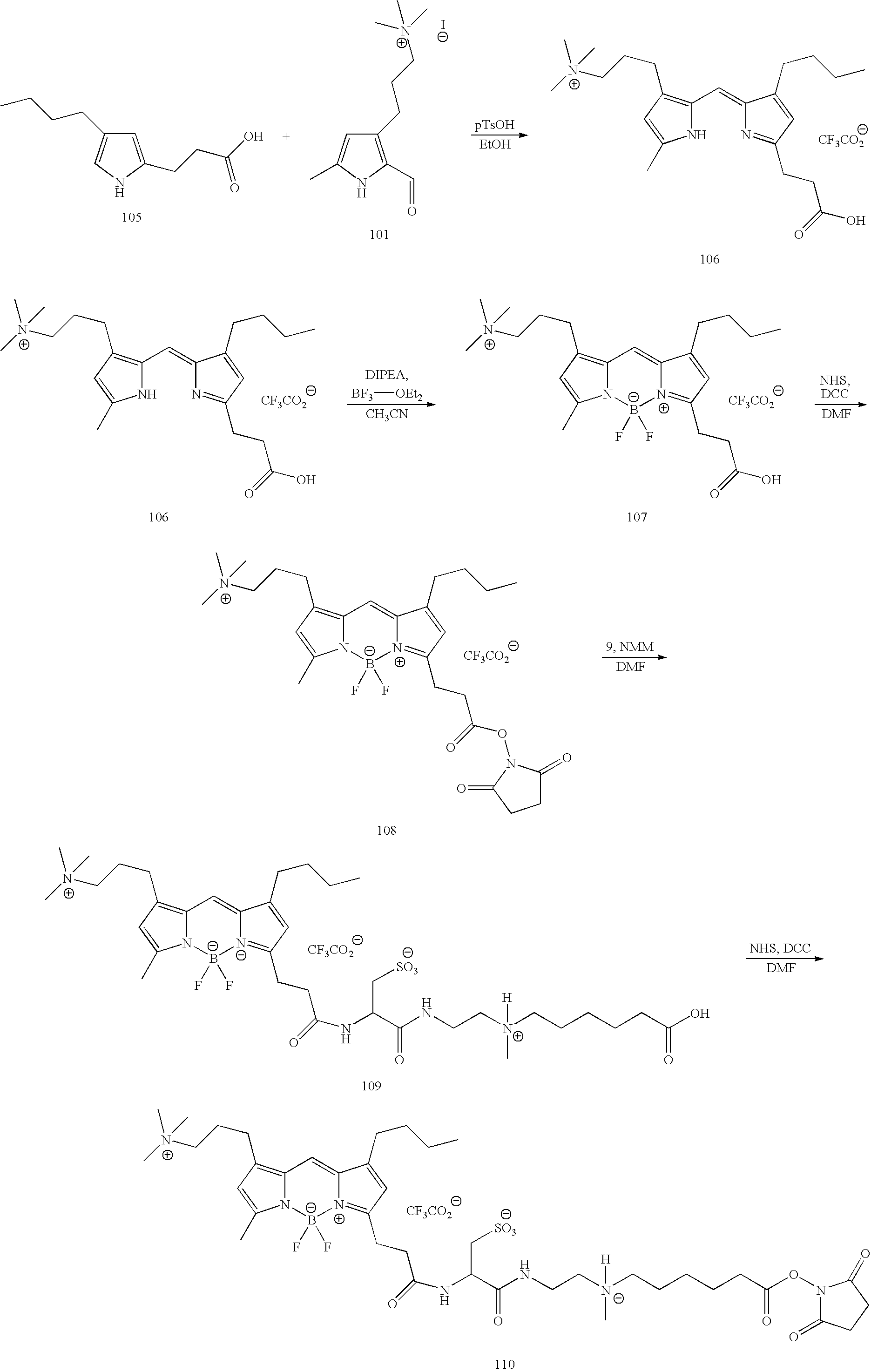

### Diethyl 3-oxohexanedioate (95)

To a 0° C. solution of 2,2-dimethyl-1,3-dioxane-4,6-dione (5 g, 34.7 mmol) in dichloromethane (175 ml) under argon and containing pyridine (5.6 ml, 69.4 mmol) was added ethyl maleonylchloride. The mixture was stirred at 0° C. for 1 hr and allowed to return to room temp over 1 hr. 1M HCL was added and the mixture was extracted with dichloromethane and the organics dried over sodium sulfate. The solvent was removed in vacuo and the resulting oil was taken up in EtOH (100 ml) and refluxed 3 hrs. The solvent was removed in vacuo and the residue dissolved in EtOAc and rinsed with a dilute sodium bicarbonate solution. The organics were dried over NaSO₄ and concentrated in vacuo. Column chromatography on Davisil provided the title compound 95.

### 2-ethyl 4-phenyl 3-(2-(ethoxycarbonyl)ethyl)-5-methyl-1H-pyrrole-2,4-dicarboxylate (96)

To a solution of diethyl 3-oxohexanedioate 95 (1.17 g, 5.41 mmol) in glacial acetic acid (5.3 ml) containing conc. HCl (15 ul), was added n-amyl nitrite (632 mg, 5.39 mmol) The temperature throughout the addition was maintained at 20°-25° C. by application of an ice bath. The reaction was stirred at room temperature over night. Phenyl 3-oxobutanoate (1.05 g) was added while the temperature was maintained below 65° C. Cooling was required for one hour. Zn dust (844 mg) was added portion wise; the reaction was heated to 100° C. and stirred three hours. The reaction mixture was poured into 75 ml of ice water with vigorous stirring and the resulting precipitate was collected by vacuum filtration. The precipitate was dissolved in MeOH and the Zn was removed by filtration. The solvent was removed and the resulting crude mixture was recrystallized from EtOH to provide the title compound 96 in (1.63 g, 3.38 mmol, 81%)

### 3-(5-methyl-1H-pyrrol-3-yl)propanoic acid (97)

To a hot solution of NaOH (1.42 g, 35.4 mmol) in ethylene glycol 35 ml was added substrate (96) the reaction was heated to reflux at 240°-260° C. bath temp. for 10 min. The reaction was poured over crushed ice and acidified by addition of 6N HCl (aq). The reaction mixture was extracted 3×25 ml EtOAc and the organic phase was washed repeatedly with water. The solvent was removed in vacuo and the crude product was taken up in 1N NaOH (aq) and brought to reflux 1 hr. The solution was cooled to room temp and acidified with 6N HCl (aq). The reaction mixture was again extracted with EtOAc and the organics washed with water. The solvent was removed in vacuo. Column Chromatography on SiO₂ provided 97 (762 mg 14.5 mmol, 41%)

### N,N-dimethyl-3-(5-methyl-1H-pyrrol-3-yl)propanamide (98)

A solution of propanoic acid (97) (230 mg, 1.5 mmol) in THF (2 ml) was treated with triethyl amine (208 ul, 1.5 mmol) at 0° C. under argon. Ethyl chloroformate was added and the mixture was stirred at 0° C. 5 min. 40% dimethylamine in H₂O (400 ul) was added and the reaction was allowed to warm to room temperature and stirred for 35 min. The solvent was removed in vacuo and the residue was taken up in EtOAc and partitioned with saturated aqueous sodium bicarbonate. The organic phase was dried over sodium sulfate, filtered, and concentrated in vacuo to provide 98 (205 mg, 1.14 mmol, 78%)

### N,N-dimethyl-3-(5-methyl-4H-pyrrol-3-yl)propan-1-amine (99)

A solution of amide 98 (210 mg, 1.16 mmol) in anhydrous THF (28 mL) was slowly added to a stirred suspension of LAH (220 mg, 5.80 mmol) in anhydrous THF (14 mL) cooled to 0° C. under argon. The mixture was stirred at ambient temperature for 2 h. The mixture was cooled to 0° C., followed by quenching with 1.5 M Na₂CO₃. Water (30 mL) was added, and the reaction mixture was extracted with EtOAc. The organic layer was washed with brine (1×) and dried (Na₂SO₄). Removal of the solvent in vacuo provided 99 as a brown oil (189 mg, 1.14 mmol, 98%)

### 3-(3-(dimethylamino)propyl)-5-methyl-1H-pyrrole-2-carbaldehyde (100)

Trimethyl orthoformate (0.350 mL, 3.20 mmol) was added to a flask containing a solution of pyrrole 99 (194 mg, 1.00 mmol) in TFA (3.5 mL) under argon at 0° C. The mixture was stirred at 0° C. for 1 h before being quenched with cold H₂O (5 mL). The mixture was basified using 1 M NaOH and the product was isolated by extraction with DCM. The organic layer was dried (Na₂SO₄) and the solvent removed in vacuo providing 100 as a yellow solid (151 mg, 0.78 mmol, 78%)

### 3-(3-(trimethylamonium iodide)propyl)-5-methyl-1H-pyrrole-2-carbaldehyde (101)

Iodomethane (1 mL) was added to a solution of amine 100 (31.1 mg, 0.160 mmol) in anhydrous DCM (3 mL). Upon addition of the iodomethane, a precipitate was noted. The suspension was stirred at ambient temperature for a further 40 min before the solvent was removed in vacuo providing 101 as a light brown solid (44.4 mg, 0.145 mmol, 91%)

### 4-butyl-1H-pyrrole-2-carbaldehyde (102)

A 1.7 M solution of t-BuLi in pentane (5.85 ml, 9.95 mmol) was added dropwise to a solution of 3-bromo-6-dimethylamino-1-azafulvene dimer (1.0 g, 2.49 mmol) in anhydrous THF (230 mL) cooled to −78° C. under argon. The solution was maintained at −78° C. for 30 m, followed by addition of 1-iodobutane (1.14 mL, 9.97 mmol). The solution was allowed to warm to −50° C. over 1 h, followed by further stirring at ambient temperature for 30 min. Saturated NaHCO₃ (44 mL) and H₂O (44 mL) were added to the solution, and the mixture was refluxed 15 h. The mixture was allowed to cool to ambient temperature, and the product was isolated by extraction with DCM. The organic layer was dried (Na₂SO₄) and the solvent removed in vacuo providing a brown residue. The crude product was purified by flash chromatography on silica gel (9:1 hexanes-ethyl acetate) providing 102 as a brown oil (538 mg, 3.56 mmol, 71%)

### (E)-Ethyl 3-(4-butyl-1H-pyrrol-2-yl)acrylate (103)

A solution of aldehyde 102 (458 mg, 3.03 mmol) and (carbethoxymethylene)-triphenylphosphorane (1.58 g, 4.54 mmol) in anhydrous benzene (30 mL) was refluxed 15 h. The solution was allowed to cool to ambient temperature, and the solvent was removed in vacuo. The crude product was purified by flash chromatography on silica gel (9:1 hexanes-ethyl acetate) providing 103 as a light brown oil (663 mg, 3.00 mmol, 99%)

### Ethyl 3-(4-butyl-1H-pyrrol-2-yl)propanoate (104)

A flask containing a suspension of acrylate 103 (560 mg, 2.53 mmol) and 10% Pd/C (270 mg, 0.255 mmol) in ethanol (10 mL) was charged with hydrogen. The suspension was stirred under hydrogen (1 atm) for 2 h. The catalyst was filtered and rinsed with ethanol. The solvent was removed in vacuo providing 104 as a colorless oil (468 mg, 2.10 mmol, 83%)

### Ethyl 3-(4-butyl-1H-pyrrol-2-yl)propionic acid (105)

A suspension of propanoate 104 (568 mg, 2.10 mmol) in 0.5 M NaOH (50 mL) was stirred at 85° C. for 1 h. The mixture was cooled to ambient temperature and acidified to pH 3 using 1 M HCl. The reaction mixture was extracted with EtOAc. The organic layer was washed with brine (1×) and dried (Na₂SO₄). Removal of the solvent in vacuo provided 105 as a brown solid (409 mg, 2.10 mmol, 100%)

### 3-(Trimethylammonium trifluoroacetate)-propyl-5-methyl-3′-butyl-5′-(3-propionic Ethyl acid)dipyrromethene (106)

Para-toluenesulfonic acid monohydrate (46.8 mg, 0.246 mmol) was added to a stirred suspension of ammonium iodide 105 (79.3 mg, 0.246 mmol) and acid 11 (8.4 mg, 0.246 mmol) in ethanol (4 mL) under ambient atmosphere and temperature. The mixture was stirred for 30 min, solvent was removed in vacuo. The crude product was purified via reverse phase HPLC using a gradient of 3:7 ([95% CH₃CN/4.9% H₂O/0.1% TFA]:[99.9% H₂O/0.1% TFA]) to 1:1 ([95% CH₃CN/4.9% H₂O/0.1% TFA]:[99.9% H₂O/0.1% TFA]) over 30 min at a flow rate of 20 mL/min, monitoring at 450 nm. The fractions containing the product were frozen, and the solvent was removed on a lyophilizer providing 106 as an orange solid (101.9 mg, 0.204 mmol, 83%)

### 4,4-Difluoro-1-butyl-7-(trimethylammonium trifluoroacetate)-propyl-5-methyl-4-bora-3a,4a,diaza-s-indacene-3-propionic acid (107)

Freshly distilled N,N-diisopropylethylamine (774 ul, 4.44 mmol) was added to a solution of dipyrromethene 106 (74 mg, 0.148 mmol) in anhydrous acetonitrile (5.0 mL) under argon at ambient temperature, and the resulting mixture was stirred at ambient temperature for 5 min. The mixture was cooled to 0° C. and BF₃.THF (114 μL, 1.268 mmol) was added. The mixture was stirred at 0° C. for 30 min before the solvent was removed in vacuo at 0° C. The crude product was purified via reverse phase HPLC using a gradient of 35:65 ([95% CH₃CN/4.9% H₂O/0.1% TFA]:[99.9% H₂O/0.1% TFA]) to 55:45 ([95% CH₃CN/4.9% H₂O/0.1% TFA]:[99.9% H₂O/0.1% TFA]) over 30 min at a flow rate of 20 mL/min, monitoring at 450 nm. The fractions containing the product were frozen, and the solvents removed on a lyophilizer providing 107 as an orange solid (26.4 mg, 0.0484 mmol, 33%)

### 4,4-Difluoro-1-butyl-7-(trimethylammonium trifluoroacetate)-propyl-5-methyl-4-bora-3a,4a,diaza-s-indacene-3-propionic acid, succinimidyl ester (108)

Anhydrous DMF (4 mL) was added to a flask containing acid 107 (21.7 mg, 0.0386 mmol), NHS (4.0 mg, 0.0348 mmol) and DCC (5.0 mg, 0.0242 mmol) under argon at ambient temperature. The mixture was stirred at ambient temperature for 6 h before the solvent was removed via the use of a lyophilizer. The crude product was purified via reverse phase HPLC using a gradient of 2:3 ([95% CH₃CN/4.9% H₂O/0.1% TFA]:[99.9% H₂O/0.1% TFA]) to 7:3 ([95% CH₃CN/4.9% H₂O/0.1% TFA]:[99.9% H₂O/0.1% TFA]) over 30 min at a flow rate of 20 mL/min, monitoring at 450 nm. The solution containing the product was frozen, and the solvents removed on a lyophilizer providing 108 as an orange solid (21.14 mg, 0.0328 mmol, 85%)

### 6-(4,4-Difluoro-1-butyl-5-(trimethylammonium trifluoroacetate)-propyl-7-methyl-4-bora-3a,4a,diaza-s-indacene-3-(N-methyl-N-(2-((R)-2-propionamido-3-sulfono-propionamido)ethyl)amino)hexanoic acid (109)

N-methylmorpholine (19 ml, 0.177 mmol) was added to a stirred mixture of succinimidyl ester 108 (21.6 mg, 0.0328 mmol) and hexanoic acid (Side chain 9) (10.0 mg, 0.0249 mmol) in anhydrous DMF (1 mL) under argon at ambient temperature. The mixture was stirred at ambient temperature for 4 h before the solvent was removed on a lyophilizer. The crude product was purified via reverse phase HPLC using a gradient of 3:7 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 450 nm. The product was collected at 14 min. The solution containing the product was frozen, and the solvents removed via the use of a lyophilizer providing 109 as an orange powder (3.7 mg, 0.00426 mmol, 49%); HRMS [M] calculated for C₃₅H₅₈BF₂N₆O₇S⁺ 755.4149. found 755.4110.

### 6-(4,4-Difluoro-1-butyl-5-(trimethylammonium trifluoroacetate)-propyl-7-methyl-4-bora-3a,4a,diaza-s-indacene-3-(N-methyl-N-(2-((R)-2-propionamido-3-sulfono-propionamido)ethyl)amino)hexanoic acid, succinimidyl ester (110)

The acid 109 (3.7 mg, 0.00426 mmol) was added to NHS (5.0 mg, 0.0434 mmol) and DCC (6.0 mg, 0.0291 mmol) in DMF (1 mL) under argon at ambient temperature. The mixture was stirred at ambient temperature for 6 h before the solvent was removed via the use of a lyophilizer. The crude product was purified via reverse phase HPLC using a gradient of 3:7 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 450 nm. The solution containing the product was frozen, and the solvents removed via the use of a lyophilizer providing 110 as an orange solid (1.5 mg, 0.00155 mmol, 36%).

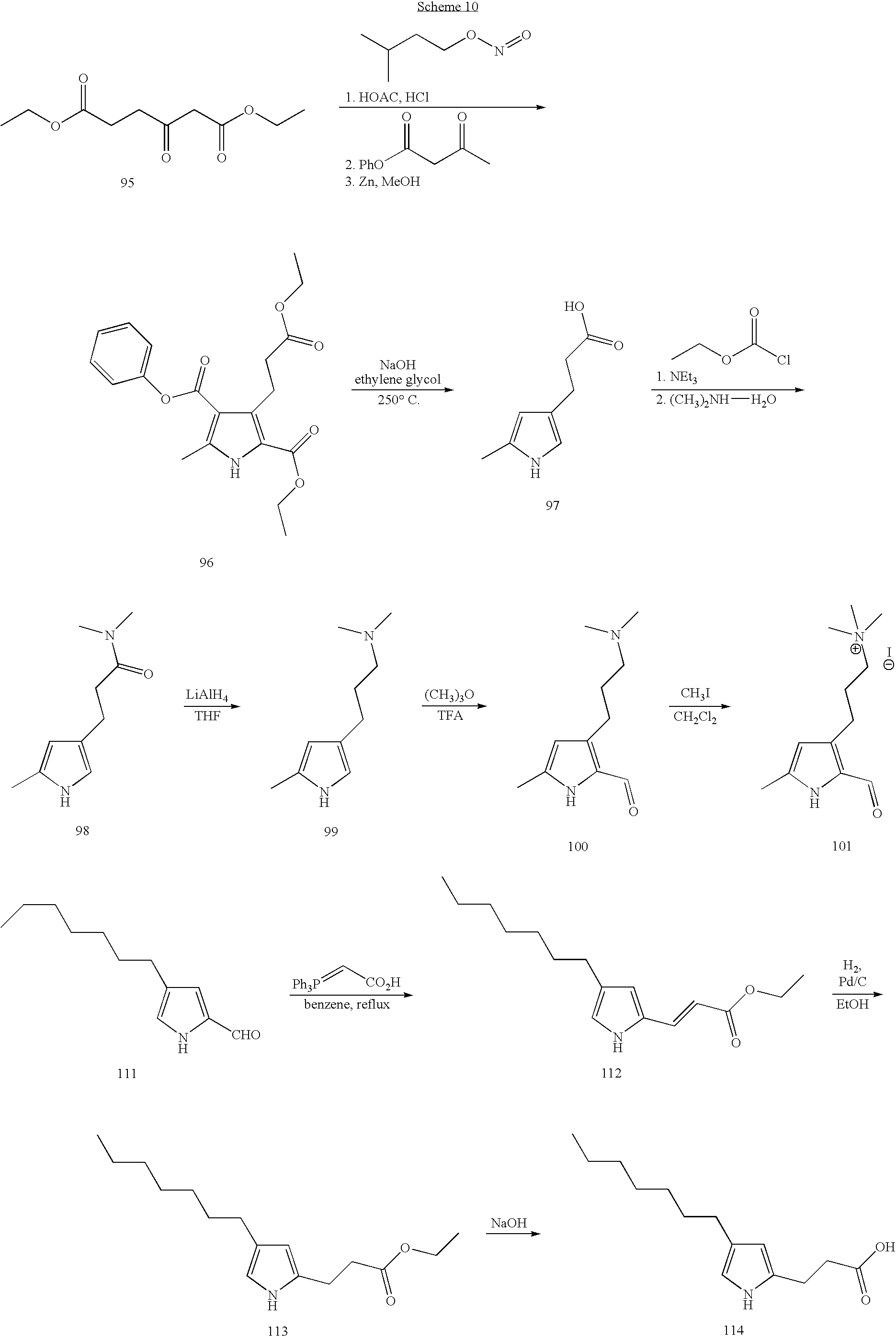

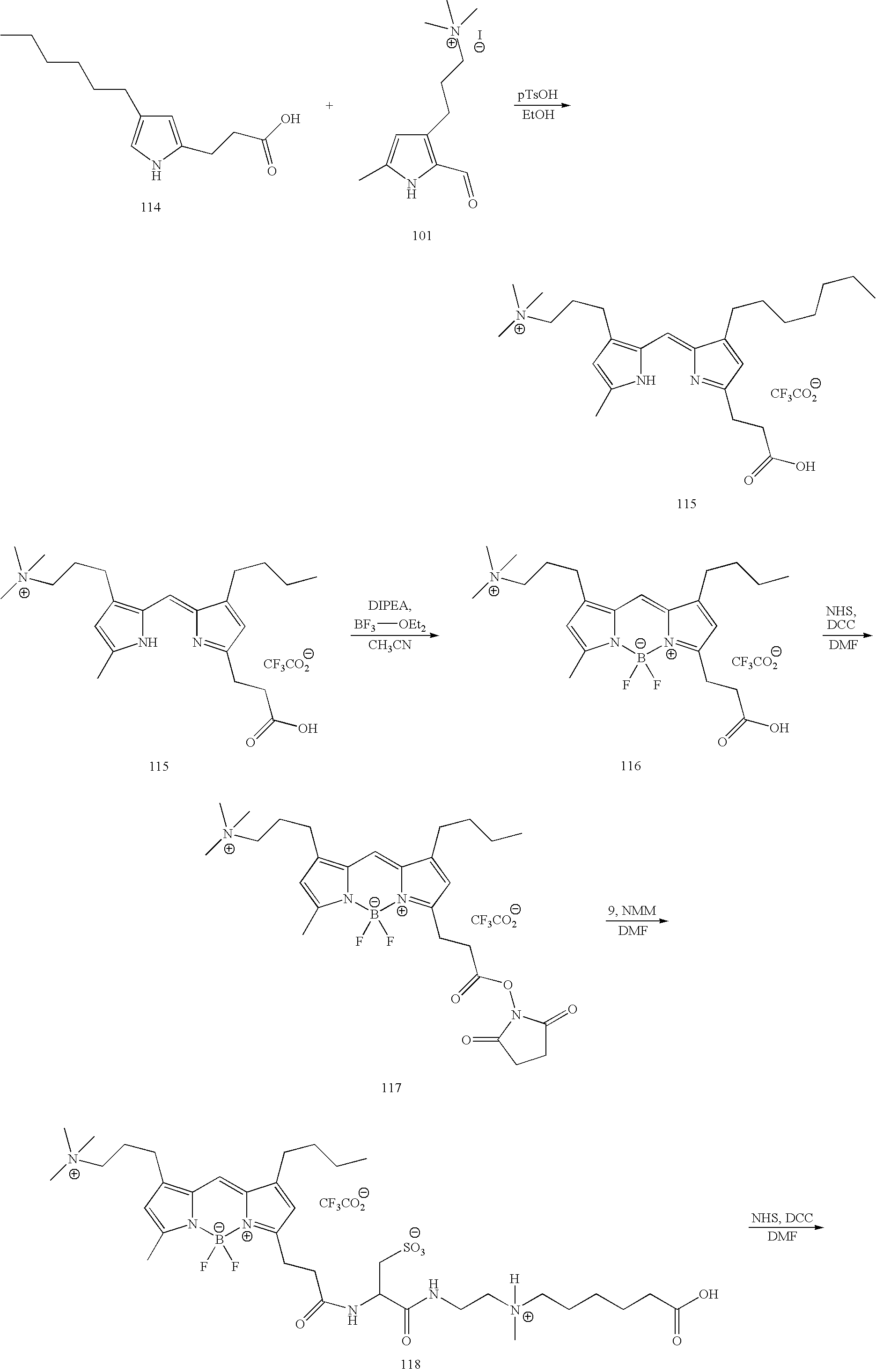

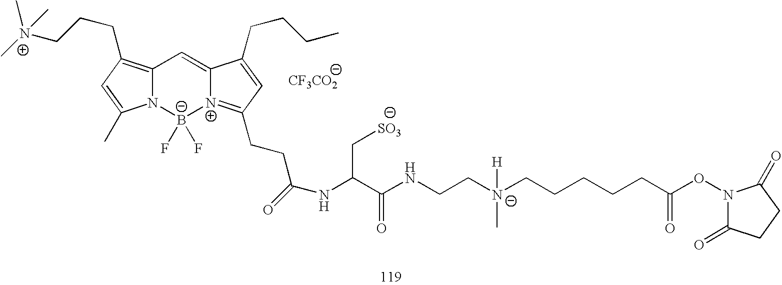

### 4-heptyl-1H-pyrrole-2-carbaldehyde (111)

A 1.7 M solution of t-BuLi in pentane (5.85 ml, 9.95 mmol) was added dropwise to a solution of 3-bromo-6-dimethylamino-1-azafulvene dimer (1.0 g, 2.49 mmol) in anhydrous THF (115 mL) cooled to −78° C. under argon. The solution was maintained at −78° C. for 30 m, followed by addition of 1-iodoheptane (1.63 mL, 9.94 mmol). The solution was allowed to warm to −50° C. over 1 h, followed by further stirring at ambient temperature for 30 min. Saturated NaHCO₃ (44 mL) and H₂O (44 mL) were added to the solution, and the mixture was refluxed 15 h. The mixture was allowed to cool to ambient temperature, and the product was isolated by extraction with DCM. The organic layer was dried (Na₂SO₄) and the solvent removed in vacuo providing a brown residue. The crude product was purified by flash chromatography on silica gel (9:1 hexanes-ethyl acetate) providing 111 as a brown oil (682 mg, 3.53 mmol, 71%)

### (E)-Ethyl 3-(4-heptyl-1H-pyrrol-2-yl)acrylate (112)

A solution of aldehyde 111 (682 mg, 3.53 mmol) and (carbethoxymethylene)-triphenylphosphorane (1.84 g, 5.28 mmol) in anhydrous benzene (35 mL) was refluxed 15 h. The solution was allowed to cool to ambient temperature, and the solvent was removed in vacuo. The crude product was purified by flash chromatography on silica gel (9:1 hexanes-ethyl acetate) providing 112 as a pale yellow solid (652 mg, 70%)

### Ethyl 3-(4-heptyl-1H-pyrrol-2-yl)propanoate (113)

A flask containing a suspension of acrylate 112 (405 mg, 1.54 mmol) and 10% Pd/C (164 mg, 0.154 mmol) in ethanol (8 mL) was charged with hydrogen. The suspension was stirred under hydrogen (1 atm) for 1.5 h. The catalyst was filtered and rinsed with ethanol. The solvent was removed in vacuo providing 113 as a yellow oil.

### Ethyl 3-(4-heptyl-1H-pyrrol-2-yl)propionic acid (114)

A suspension of propanoate 113 in 0.5 M NaOH (50 mL) was stirred at 85° C. for 1 h.

The mixture was cooled down to ambient temperature and acidified to pH 3 using 1 M HCl. The product was isolated by extraction with EtOAc. The organic layer was washed with brine (1×) and dried (Na₂SO₄). Removal of the solvent in vacuo provided 114.

### 3-(Trimethylammonium trifluoroacetate)-propyl-5-methyl-3′-heptyl-5′-(3-propionic Ethyl acid)dipyrromethene (115)

Para-toluenesulfonic acid monohydrate (34.6 mg, 0.182 mmol) was added to a stirred suspension of ammonium iodide 114 (58.8 mg, 0.182 mmol) and acid 101 (43.3 mg, 0.182 mmol) in ethanol (3 mL) under room atmosphere at ambient temperature. The mixture was stirred for 30 min. solvent was removed in vacuo. The crude product was purified with reverse phase HPLC using a gradient of 3:7 ([95% CH₃CN/4.9% H₂O/0.1% TFA]:[99.9% H₂O/0.1% TFA]) to 1:1 ([95% CH₃CN/4.9% H₂O/0.1% TFA]:[99.9% H₂O/0.1% TFA]) over 30 min at a flow rate of 20 mL/min, monitoring at 450 nm. The solution containing the product was frozen, and the solvent removed on a lyophilizer providing 115 as a solid (75.5 mg, 0.139 mmol, 76%).

### 4,4-Difluoro-1-heptyl-7-(trimethylammonium trifluoroacetate)-propyl-5-methyl-4-bora-3a,4a,diaza-s-indacene-3-propionic acid (116)

Freshly distilled N,N-diisopropylethylamine (600 ul, 0.742 mmol) was added to a solution of dipyrromethene 115 (75.5 mg, 0.139 mmol) in anhydrous acetonitrile (5.0 mL) under argon at ambient temperature, and the resulting mixture was stirred at ambient temperature for 5 min. The mixture was cooled to 0° C. and BF₃.THF (125 μL, 1.268 mmol) was added. The mixture was stirred at 0° C. for 30 min before the solvent was removed in vacuo at 0° C. The crude product was purified with reverse phase HPLC using a gradient of 35:65 ([95% CH₃CN/4.9% H₂O/0.1% TFA]:[99.9% H₂O/0.1% TFA]) to 55:45 ([95% CH₃CN/4.9% H₂O/0.1% TFA]:[99.9% H₂O/0.1% TFA]) over 30 min at a flow rate of 20 mL/min, monitoring at 450 nm. The solution containing the product was frozen, and the solvents removed on a lyophilizer providing 116 (6.0 mg, 7%)

### 4,4-Difluoro-1-heptyl-7-(trimethylammonium trifluoroacetate)-propyl-5-methyl-4-bora-3a,4a,diaza-s-indacene-3-propionic acid, succinimidyl ester (117)

Anhydrous DMF (2 mL) was added to a flask containing acid 116 (6.0 mg, 0.0102 mmol), NHS (15.0 mg, 0.130 mmol) and DCC (12.0 mg, 0.0582 mmol) under argon at ambient temperature. The mixture was stirred at ambient temperature for 7 h before the solvent was removed via the use of a lyophilizer. The crude product was purified via reverse phase HPLC using a gradient of 2:3 ([95% CH₃CN/4.9% H₂O/0.1% TFA]:[99.9% H₂O/0.1% TFA]) to 7:3 ([95% CH₃CN/4.9% H₂O/0.1% TFA]:[99.9% H₂O/0.1% TFA]) over 30 min at a flow rate of 20 mL/min, monitoring at 450 nm. The solution containing the product was frozen, and the solvents removed on a lyophilizer providing 117 as an orange solid (5.0 mg, 0.00728 mmol, 71%).

### 6-(4,4-Difluoro-1-heptyl-5-(trimethylammonium trifluoroacetate)-propyl-7-methyl-4-bora-3a,4a,diaza-s-indacene-3-(N-methyl-N-(2-((R)-2-propionamido-3-sulfono-propionamido)ethyl)amino)hexanoic acid (118)

N-methylmorpholine (15.6 μl, 0.145 mmol) was added to a stirred mixture of succinimidyl ester 117 (5.0 mg, 0.00728 mmol) and hexanoic acid (Side chain 9) (8.8 mg, 0.0219 mmol) in anhydrous DMF (1 mL) under argon at ambient temperature. The mixture was stirred at ambient temperature for 4 h before the solvent was removed via the use of a lyophilizer. The crude product was purified via reverse phase HPLC using a gradient of 2:8 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 450 nm. The solution containing the product was frozen, and the solvents removed on a lyophilizer providing 116 as an orange powder (4.2 mg, 0.00461 mmol, 63%); HRMS [M+] calculated for C₃₈H₆₄F₂N₆O₇S⁺ 797.4618. found 797.4568.

### 6-(4,4-Difluoro-1-hexyl-5-(trimethylammonium trifluoroacetate)-propyl-7-methyl-4-bora-3a,4a,diaza-s-indacene-3-(N-methyl-N-(2-((R)-2-propionamido-3-sulfono-propionamido)ethyl)amino)hexanoic acid, succinimidyl ester (119)

The acid 118 (4.2 mg, 0.00461 mmol) was added to NHS (5.3 mg, 0.0460 mmol) and DCC (7.6 mg, 0.0368 mmol) in DMF (1 mL) under argon at ambient temperature. The mixture was stirred at ambient temperature for 24 h before the solvent was removed via the use of a lyophilizer. The crude product was purified twice via reverse phase HPLC using a gradient of 3:7 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 450 nm. The solution containing the product was frozen, and the solvents removed via the use of a lyophilizer providing 119 as an orange solid (1.5 mg, 0.00149 mmol, 32%).

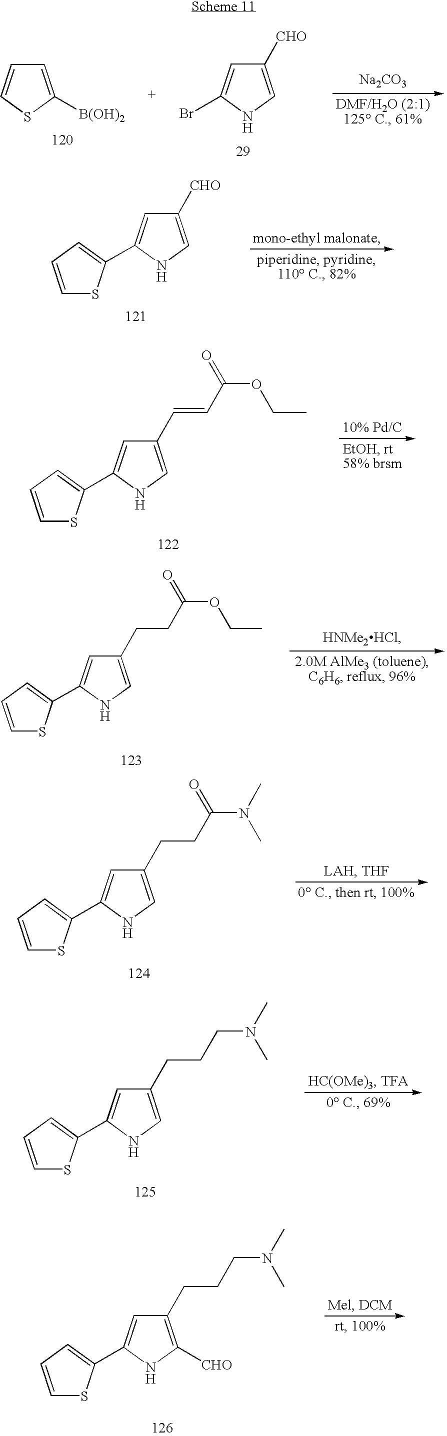

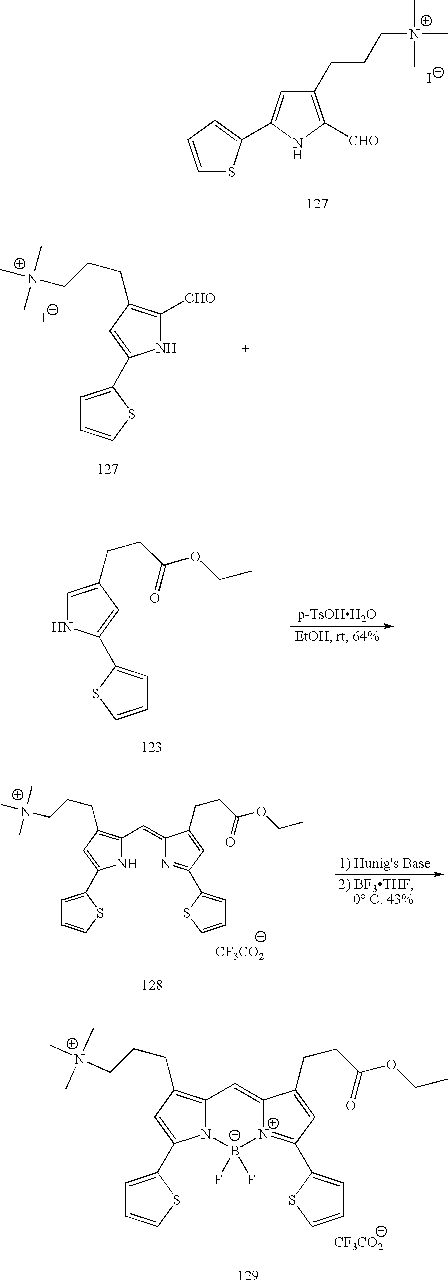

5-(2-thienyl)-1H-pyrrole-3-carboxaldehyde (121): A solution of Na₂CO₃ (3.5 g, 33.0 mmol) in degassed water (22.4 mL) was added to a flask containing a suspension of 5-bromo-1H-pyrrole-3-carboxaldehyde (29) (1.83 g, 10.5 mmol) and tetrakis(triphenylphosphine)palladium(0) (570 mg, 0.493 mmol) in degassed DMF (66 mL) under argon at ambient temperature, followed by addition of a solution of 2-thiopheneboronic acid (120) (1.96 g, 15.3 mmol) in degassed DMF (34.7 mL). The mixture was refluxed at 125° C. for 15 h. The flask was cooled down to ambient temperature and poured into DCM (155 mL). The mixture was washed with H₂O (6×180 mL) and the organic phase was dried (Na₂SO₄). Removal of the solvent in vacuo provided the crude product which was purified by flash chromatography on silica gel (7:3 hexanes-ethyl acetate) providing 121 as a yellow solid (1.13 g, 6.40 mmol, 61%): mp 198-199° C.; R_{f}: 0.20 (7:3 hexanes-ethyl acetate); FTIR (CH₂Cl₂): 1637(s); ¹H NMR (500 MHz, CD₃OD) δ 6.70 (d, J=1.5 Hz, 1H), 7.03 (dd, J=5, 3.5 Hz, 1H), 7.23 (dd, J=3.5, 0.5 Hz, 1H), 7.30 (dd, J=5, 0.5 Hz, 1H), 7.59 (d, J=1.5 Hz, 1H), 9.64 (s, 1H); ¹³C NMR (125 MHz, CD₃OD) δ 104.46, 123.79, 125.13, 128.77, 128.84, 131.24, 136.11, 187.96; HRMS-ES (m/z): [M+H] calcd for C₉H₈NOS⁺ 178.0321. found 178.0354.

(E)-Ethyl 3-(5-(thiophen-2-yl)-1H-pyrrol-3-yl)acrylate (122): Piperidine (0.13 mL, 1.32 mmol) and mono-ethyl-malonate (5.0 mL, 42.3 mmol) were added to a flask containing a solution of aldehyde 121 (1.13 g, 6.38 mmol) in anhydrous pyridine (6.6 mL) under argon at ambient temperature. The mixture was stirred at 110° C. for 5.5 h before being cooled and quenched with H₂O (10 mL). The mixture was acidified to pH 3 using 1M HCl, followed by isolation of the product by extraction with EtOAc. The organic layer was dried (Na₂SO₄) and the solvent removed in vacuo. The crude product was purified by flash chromatography on silica gel (85:15 hexanes-ethyl acetate) providing 122 as a white solid (1.30 g, 5.26 mmol, 82%): mp 104-105° C.; R_{f}: 0.32 (4:1 hexanes-ethyl acetate); FTIR (CH₂Cl₂): 1615(s), 1684(s), 3261(s); ¹H NMR (500 MHz, CD₃OD) δ 1.31 (t, J=7 Hz, 3H), 4.20 (q, J=7 Hz, 2H), 6.10 (d, J=16 Hz, 1H), 6.58 (d, J=1.5 Hz, 1H), 7.02 (dd, J=9, 5 Hz, 1H), 7.10 (d, J=1.5 Hz, 1H), 7.18 (dd, J=3.5, 1.5 Hz, 1H), 7.24 (dd, J=5, 1 Hz, 1H), 7.61 (d, J=16 Hz, 1H); ¹³C NMR (125 MHz, CD₃OD) δ 14.82, 61.28, 104.46, 113.30, 122.79, 122.88, 124.21, 124.52, 128.70, 130.43, 137.05, 140.95, 170.23; HRMS-ES (m/z): [M+H] calcd for C₁₃H₁₄NO₂S⁺ 248.0740. found 248.0781.

Ethyl 3-(5-(thiophen-2-yl)-1H-pyrrol-3-yl)propanoate (123): A flask containing a suspension of acrylate 122 (915 mg, 3.70 mmol) and 10% Pd/C (1.00 g, 0.94 mmol) in ethanol (40 mL) was charged with hydrogen. The suspension was stirred under hydrogen (1 atm) for 2 h. The catalyst was filtered and rinsed with ethanol. The solvent was removed in vacuo. The crude product was purified by flash chromatography on silica gel (85:15 hexanes-ethyl acetate) providing 123 as a white solid (327 mg, 1.31 mmol, 58% brsm): mp 39-40° C.; R_{f}: 0.26 (9:1 hexanes-ethyl acetate); FTIR (CH₂Cl₂): 1714(s), 3366(s); ¹H NMR (500 MHz, CD₃OD) δ 1.20 (t, J=7 Hz, 3H), 2.54 (t, J=7.5 Hz, 2H), 2.74 (t, J=7.5 Hz, 2H), 4.09 (q, J=7 Hz, 2H), 6.16 (d, J=2 Hz, 1H), 6.53 (s, 1H), 6.93 (dd, J=5, 3.5 Hz, 1H), 7.03 (dd, J=3.5, 1 Hz, 1H), 7.09 (dd, J=5, 1 Hz, 1H); ¹³C NMR (125 MHz, CD₃OD) δ 14.65, 23.62, 37.12, 61.54, 107.16, 117.27, 121.19, 122.80, 124.54, 127.95, 128.50, 138.44, 175.52; HRMS-ES (m/z): [M+H] calcd for C₁₃H₁₆NO₂S⁺ 250.0896. found 250.0918.

### N,N-Dimethyl-3-(5-(thiophen-2-yl)-1H-pyrrol-3-yl)propanamide (124)

A solution of 2.0 AlMe₃ in toluene (1.44 mL, 2.86 mmol) was added to a flask containing a solution of dimethylamine hydrochloride (233 mg, 2.86 mmol) in anhydrous benzene (10.6 mL) under argon at ambient temperature. The mixture was stirred at ambient temperature for 1 h before addition of a solution of propanoate 123 (354 mg, 1.42 mmol) in anhydrous benzene (10.6 mL). The mixture was refluxed for 15 h. The mixture was cooled down to ambient temperature and quenched by slow addition of 1 M HCl (10 mL). Water (25 mL) was added, and the product was isolated by extraction with EtOAc. The organic layer was dried (MgSO₄) and the solvent removed in vacuo providing 124 as a white solid (337 mg, 1.36 mmol, 96%): mp 107-109° C.; R_{f}: 0.42 (ethyl acetate); FTIR (CH₂Cl₂): 1631(s); ¹H NMR (500 MHz, CD₃OD) δ 2.59 (t, J=8 Hz, 2H), 2.73 (t, J=8 Hz, 2H), 2.90 (s, 3H), 2.95 (s, 3H), 6.18 (s, 1H), 6.56 (s, 1H), 6.95 (dd, J=5, 3.5 Hz, 1H), 7.04 (d, J=3.5 Hz, 1H), 7.12 (d, J=5 Hz, 1H); ¹³C NMR (125 MHz, CD₃OD) δ 24.08, 35.92, 36.16, 38.03, 107.29, 117.40, 121.21, 122.82, 124.88, 128.02, 128.55, 138.53, 175.77; HRMS-ES (m/z): [M+H] calcd for C₁₃H₁₇N₂OS⁺ 249.1056. found 249.1072.

N,N-Dimethyl-3-(5-(thiophen-2-yl)-1H-pyrrol-3-yl)propan-1-amine (125): A solution of amide 124 (296 mg, 1.19 mmol) in anhydrous THF (43 mL) was added dropwise to a flask containing a suspension of LAH (280 mg, 7.37 mmol) in anhydrous THF (30 mL) under argon cooled to 0° C. The mixture was stirred at ambient temperature for 3 h before being cooled to 0° C. and quenched with 1.5 M Na₂CO₃ (5 mL). Water (30 mL) was added, and the product was isolated by extraction with EtOAc. The organic layer was dried (Na₂SO₄) and the solvent removed in vacuo providing 125 as a light brown residue (278 mg, 1.19 mmol, 100%): R_{f}: 0.20 (3:2 dichloromethane-methanol); FTIR (CH₂Cl₂): 1265(s), 2857(s), 2931(s), 3053(s); ¹H NMR (500 MHz, CD₃OD) δ 1.74 (p, J=7.5 Hz, 2H), 2.20 (s, 6H), 2.33 (m, 2H), 2.44 (t, J=7.5 Hz, 2H), 6.16 (d, J=0.5 Hz, 1H), 6.53 (s, 1H), 6.94 (dd, J=5, 3.5 Hz, 1H), 7.04 (d, J=3.5 Hz, 1H), 7.09 (d, J=5 Hz, 1H); ¹³C NMR (125 MHz, CD₃OD) δ 25.91, 29.87, 45.52, 60.48, 107.28, 117.24, 121.07, 122.68, 125.59, 127.92, 128.53, 138.64; HRMS-ES (m/z): [M+H] calcd for C₁₃H₁₉N₂S⁺ 235.1263. found 235.1280.

3-(3-(dimethylamino)propyl)-5-(thiophen-2-yl)-1H-pyrrole-2-carboxaldehyde (126): A flask containing a solution of amine 125 (280 mg, 1.19 mmol) in TFA (2.3 mL) under argon was cooled to 0° C. Trimethyl orthoformate (0.4 mL, 3.65 mmol) was added to the solution, and the mixture was stirred at 0° C. for 20 min before being quenched with cold H₂O (10 mL). The mixture was basified using 5 M NaOH and the product isolated by extraction with EtOAc. The organic layer was dried (MgSO₄) and the solvent removed in vacuo. The crude product was purified by flash chromatography on silica gel (7:3 dichloromethane-methanol) providing 126 as a red residue (216 mg, 0.824 mmol, 69%): FTIR (CH₂Cl₂): 1468(s), 1639(s), 2928(s); ¹H NMR (500 MHz, CD₃OD) δ 1.86 (p, J=7.5 Hz, 2H), 2.60 (s, 6H), 2.40 (m, 2H), 2.82 (t, J=7.5 Hz, 2H), 6.42 (s, 1H), 7.09 (dd, J=5, 3.5 Hz, 1H), 7.43 (dd, J=5, 1 Hz, 1H), 7.47 (dd, J=3.5, 1 Hz, 1H), 9.56 (s, 1H); ¹³C NMR (125 MHz, CD₃OD) δ 24.20, 29.14, 30.87, 45.01, 59.77, 110.96, 125.88, 126.97, 129.24, 130.98, 135.35, 136.11; HRMS-ES (m/z): [M+H] calcd for C₁₃H₁₉N₂S⁺ 263.1213. found 263.1206.

### Trimethyl-(3-(2-formyl-5-(2-thienyl)-1H-3-pyrrolyl)-propyl)-ammonium iodide (127)

An excess of iodomethane (1 mL) was added to a flask containing a solution of aldehyde (126) (216 mg, 0.823 mmol) in anhydrous DCM (5 mL) under argon at ambient temperature. The mixture was allowed to stir at ambient temperature for 30 min. The solvent was removed in vacuo providing 127 as a red solid (332 mg, 0.821 mmol, 100%): ¹H NMR (500 MHz, CD₃OD) δ 2.15-2.21 (m, 2H), 2.92 (t, J=7 Hz, 2H), 3.14 (s, 9H), 3.42 (m, 2H), 6.53 (s, 1H), 7.11 (dd, J=5, 3.5 Hz, 1H), 7.44 (d, J=5 Hz, 1H), 7.47 (d, J=3.5 Hz, 1H), 9.62 (s, 1H); ¹³C NMR (125 MHz, CD₃OD) δ 23.57, 25.49, 53.86, 53.89, 53.92, 67.58, 111.16, 125.95, 127.05, 129.26, 131.06, 135.25, 136.06; HRMS-ES (m/z): [M] calcd for C₁₅H₂₁N₂OS⁺ 277.1369. found 277.1369.

5-(2-thienyl)-3-(trimethylammonium trifluoroacetate)-propyl-ethyl-(3′-methyl-5′-(2-thienyl))propanoate dipyrromethene (128): Para-toluenesulfonic acid monohydrate (27 mg, 0.142 mmol) was added to a stirred suspension of ammonium iodide 127 (56.0 mg, 0.139 mmol) and propanoate 123 (35.0 mg, 0.140 mmol) in ethanol (10 mL) under normal atmosphere at ambient temperature. The mixture was stirred at ambient temperature for 30 min before removal of the solvent in vacuo. The crude product was purified via reverse phase HPLC using a gradient of 3:7 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 420 nm. The product was collected at 15.5 min. The solution containing the product was frozen, and the solvents removed via the use of a lyophilizer providing 128 as a blue solid (55 mg, mmol, 64%): ¹H NMR (500 MHz, CD₃OD) δ 1.20 (t, J=7 Hz, 3H), 2.30 (m, 2H), 2.81 (t, J=7 Hz, 2H), 2.99 (t, J=7 Hz, 2H), 3.18 (t, J=7 Hz, 2H), 3.19 (s, 9H), 3.52 (m, 2H), 4.09 (q, J=7 Hz, 2H), 6.98 (s, 1H), 7.07 (s, 1H), 7.26 (d, J=3.5 Hz, 1H), 7.28 (d, J=3.5 Hz, 1H), 7.55 (s, 1H), 7.81-7.83 (m, 2H), 7.95 (t, J=3.5 Hz, 2H); ¹³C NMR (125 MHz, CD₃OD) δ 14.64, 22.66, 24.10, 24.99, 35.66, 53.87, 53.90, 53.93, 62.03, 67.30, 116.93, 117.00, 120.90, 130.55, 131.21 131.49, 131.62, 131.67, 132.93, 132.99, 133.20, 148.95, 149.16, 150.41, 151.64, 174.47; HRMS [M] calcd for C₂₈H₃₄N₃O₂S₂⁺ 508.2087. found 508.2009.

4,4-difluoro-3,5-di(2-thienyl)-7-(trimethylammonium trifluoroacetate)-propyl-4-bora-3a,4a-diaza-s-indacene-1-ethylpropanoate (129): Freshly distilled N,N-diisopropylethylamine (600 μL, 3.44 mmol) was added to a solution of dipyrromethene 128 (51.0 mg, 0.0820 mmol) dissolved in anhydrous acetonitrile (4 mL) under argon at ambient temperature, and the solution was stirred at ambient temperature for 5 min. The solution was cooled to 0° C. and BF₃.THF (74 μL, 0.671 mL) was added. The mixture was stirred at 0° C. for 30 min before the solvent was removed in vacuo at 0° C. The crude product was purified via reverse phase HPLC using a gradient of 35:65 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 40 min at a flow rate of 20 mL/min, monitoring at 420 nm. The product was collected at 23 min. The solution containing the product was frozen, and the solvents removed via the use of a lyophilizer providing 129 as a blue solid (23.4 mg, 0.0349 mmol, 42.6%): R_{f}: 0.37 (90% acetonitrile in water); ¹H NMR (500 MHz, CD₃OD) δ 1.20 (t, J=7 Hz, 3H), 2.07 (m, 2H), 2.62-2.66 (m, 4H), 2.92 (t, J=7 Hz, 2H), 3.07 (s, 9H), 3.27 (m, 2H), 4.07 (q, J=7 Hz, 2H), 6.72 (s, 1H), 6.75 (s, 1H), 7.19 (d, J=4.5 Hz, 1H), 7.20 (d, J=4.5 Hz, 1H), 7.40 (s, 1H), 7.65 (d, J=4.5 Hz, 2H), 8.13 (t, J=4.5 Hz, 2H); ¹³C NMR (125 MHz, CD₃OD) δ 14.68, 22.02, 23.30, 24.82, 35.58, 53.77, 53.80, 53.83, 61.91, 67.40, 120.14, 121.02, 129.97, 130.01, 131.01, 131.15, 132.41, 132.47, 132.52, 132.57, 132.63, 135.37, 135.42, 136.37, 139.69, 144.91, 146.27, 150.52, 150.85, 174.46; HRMS [M] calcd for C₂₈H₃₃BF₂N₃O₂S₂⁺ 556.2075. found 556.2118.

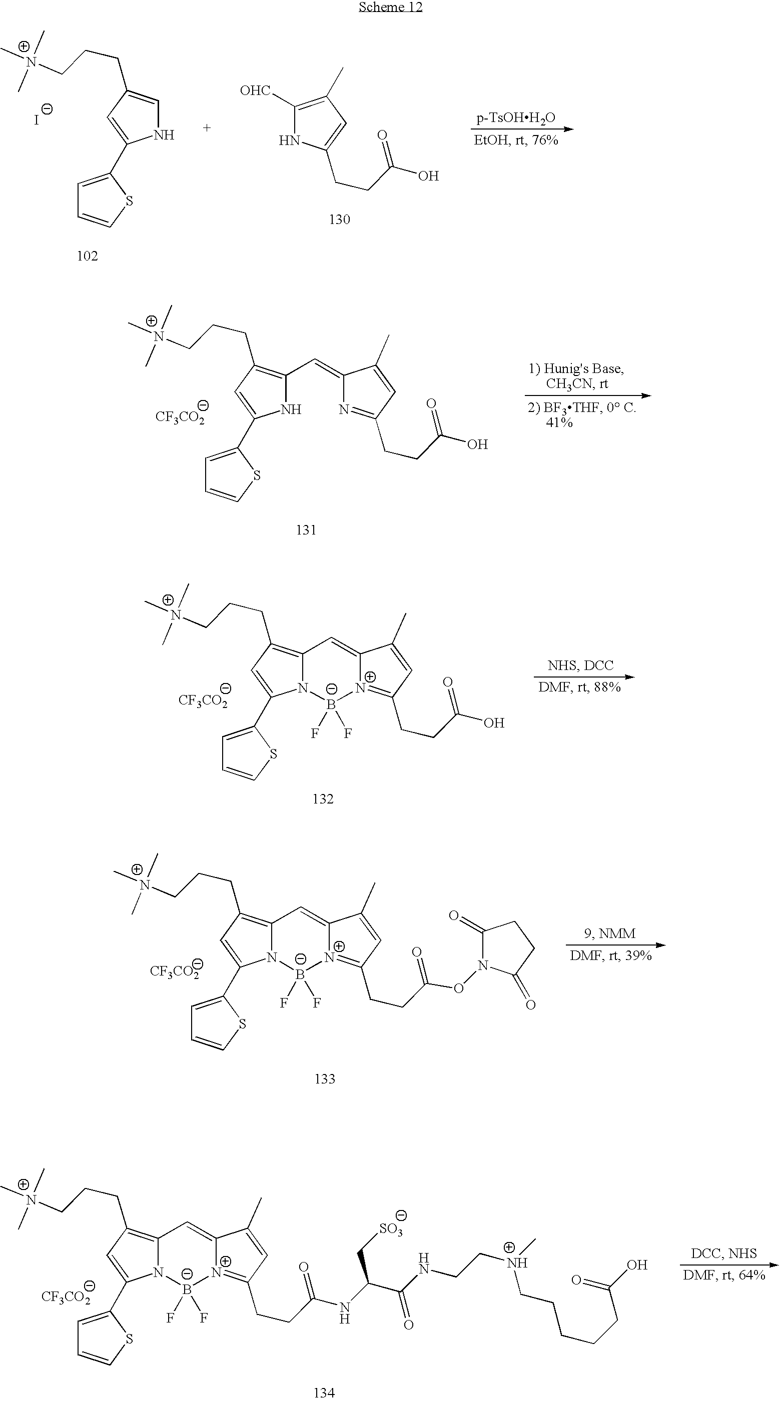

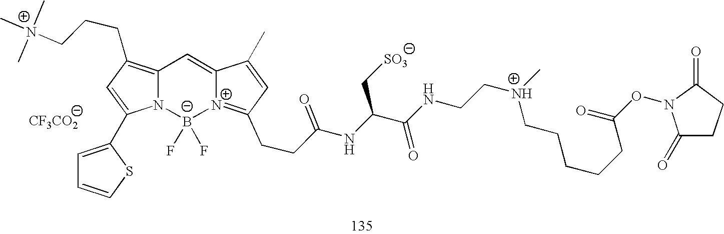

5-(2-Thienyl)-3-(trimethylammonium trifluoroacetate)-propyl-3′-methyl-5′-(3-propionic acid)dipyrromethene (131): Para-toluenesulfonic acid monohydrate (20.0 mg, 0.105 mmol) was added to a stirred suspension of ammonium iodide 102 (40.0 mg, 0.106 mmol) and acid 130 (19.3 mg, 0.106 mmol) in ethanol (5 mL) under normal atmosphere at ambient temperature. The mixture was stirred at ambient temperature for 30 min before removal of the solvent in vacuo. The crude product was purified via reverse phase HPLC using a gradient of 1:4 ([95% CH₃CN/4.9% H₂O/0.1% TFA]:[99.9% H₂O/0.1% TFA]) to 1:1 ([95% CH₃CN/4.9% H₂O/0.1% TFA]:[99.9% H₂O/0.1% TFA]) over 30 min at a flow rate of 20 mL/min, monitoring at 420 nm. The product was collected at 13 min. The solution containing the product was frozen, and the solvents removed via the use of a lyophilizer providing 131 as a red solid (42.2 mg, 0.0803 mmol, 76%): ¹H NMR (500 MHz, CD₃OD) δ 2.26 (m, 2H), 2.46 (s, 3H), 2.82 (t, J=8 Hz, 2H), 2.95 (t, J=7.5 Hz, 2H), 3.14 (t, J=8 Hz, 2H), 3.18 (s, 9H), 3.50 (m, 2H), 6.51 (s, 1H), 7.06 (s, 1H), 7.27 (dd, J=5, 4 Hz, 1H), 7.50 (s, 1H), 7.80 (d, J=5 Hz, 1H), 7.94 (d, J=4 Hz, 1H); ¹³C NMR (125 MHz, CD₃OD) δ 12.39, 23.91, 24.78, 25.15, 33.04, 53.84, 53.87, 53.90, 67.26, 116.32, 118.60, 122.38, 130.06, 130.40, 130.75, 131.30, 132.58, 133.07, 148.46, 150.00, 150.42, 160.82, 175.50; HRMS [M] calcd for C₂₃H₃₀N₃O₂S⁺ 412.2053. found 412.2037.

4,4-Difluoro-1-methyl-5-(2-thienyl)-7-(3-trimethylammonium trifluoroacetate)-propyl-4-bora-3a,4a,diaza-s-indacene-3-propionic acid (132): Freshly distilled N,N-diisopropylethylamine (0.500 mL, 2.87 mmol) was added to a stirred solution of dipyrromethene 131 (42.2 mg, 0.0803 mmol) in anhydrous acetonitrile (4.0 mL) under argon at ambient temperature, and the resulting mixture was stirred at ambient temperature for 5 min. The mixture was cooled to 0° C. and BF₃.THF (70.0 μL, 0.634 mmol) was added. The mixture was stirred at 0° C. for 30 min before the solvent was removed in vacuo at 0° C. The crude product was purified via reverse phase HPLC using a gradient of 1:4 ([95% CH₃CN/4.9% H₂O/0.1% TFA]:[99.9% H₂O/0.1% TFA]) to 1:1 ([95% CH₃CN/4.9% H₂O/0.1% TFA]:[99.9% H₂O/0.1% TFA]) over 30 min at a flow rate of 20 mL/min, monitoring at 420 nm. The product was collected at 26 min. The solution containing the product was frozen, and the solvents removed via the use of a lyophilizer providing 132 as a red solid (18.7 mg, 0.0326 mmol, 41%): ¹H NMR (500 MHz, CD₃OD) δ 2.16 (m, 2H), 2.32 (s, 3H), 2.73 (t, J=7.5 Hz, 2H), 2.79 (t, J=7.5 Hz, 2H), 3.14 (s, 9H), 3.22 (t, J=7.5 Hz, 2H), 3.40 (m, 2H), 6.30 (s, 1H), 6.77 (s, 1H), 7.17 (dd, J=4.5, 3.5 Hz, 1H), 7.50 (s, 1H), 7.60 (d, J=4.5 Hz, 1H), 8.06 (d, J=3.5 Hz, 1H); ¹³C NMR (125 MHz, CD₃OD) δ 11.50, 23.19, 25.10, 25.29, 33.74, 53.79, 53.82, 53.85, 67.40, 119.32, 120.02, 122.27, 129.77, 130.18, 131.87, 131.92, 131.98, 135.42, 135.70, 135.90, 144.56, 144.82, 149.90, 162.54, 176.13; HRMS [M] calcd for C₂₃H₂₉BF₂N₃O₂S⁺ 460.2040. found 460.2024.

4,4-Difluoro-1-methyl-5-(2-thienyl)-7-(3-trimethylammonium trifluoroacetate)-propyl-4-bora-3a,4a,diaza-s-indacene-3-propionic acid, succinimidyl ester (133): Anhydrous DMF (4 mL) was added to a flask containing acid 132(18.7 mg, 32.6 μmol), NHS (35.0 mg, 0.304 mmol) and DCC (55.0 mg, 0.266 mmol) under argon at ambient temperature. The mixture was stirred at ambient temperature for 15 h before the solvent was removed via the use of a lyophilizer. The crude product was purified via reverse phase HPLC using a gradient of 3:7 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 420 nm. The product was collected at 21 min. The solution containing the product was frozen, and the solvents removed via the use of a lyophilizer providing 133 as a red solid (19.3 mg, 28.8 mmol, 88%): R_{f}: 0.38 (9:1 acetonitrile-water); ¹H NMR (500 MHz, CD₃OD) δ 2.11 (m, 2H), 2.28 (s, 3H), 2.73 (t, J=7.5 Hz, 2H), 2.83 (s, 4H), 3.08 (t, J=7.5 Hz, 2H), 3.11 (s, 9H), 3.33-3.37 (m, 4H), 6.34 (s, 1H), 6.76 (s, 1H), 7.17 (dd, J=5.5, 3.5 Hz, 1H), 7.48 (s, 1H), 7.62 (d, J=5 Hz, 1H), 8.07 (d, J=3.5 Hz, 1H); ¹³C NMR (125 MHz, CD₃CN) δ 11.75, 22.88, 24.46, 24.68, 26.53, 30.34, 54.05, 54.08, 54.11, 66.89, 119.72, 119.87, 123.32, 129.91, 131.02, 131.90, 131.96, 134.90, 135.22, 135.69, 144.54, 145.45, 149.90, 159.71, 169.39, 171.18; HRMS [M] calcd for C₂₇H₃₂BF₂N₄O₄S⁺ 557.2201. found 557.2202.

6-(4,4-Difluoro-1-methyl-5-(2-thienyl)-7-(trimethylammonium trifluoroacetate)-propyl-4-bora-3a,4a,diaza-s-indacene-3-(N-methyl-N-(2-((R)-2-propionamido-3-sulfono-propionamido)ethyl)amino)hexanoic acid (134): N-methylmorpholine (50 μL, 0.465 mmol) was added to a solution of succinimidyl ester 133 (17.5 mg, 26.1 μmol) and hexanoic acid 9 (31.0 mg, 77.3 μmol) in anhydrous DMF under argon at ambient temperature. The mixture was stirred at ambient temperature for 4 h before the solvent was removed via the use of a lyophilizer. The crude product was purified via reverse phase HPLC using a gradient of 1:4 ([95% CH₃CN/4.9% H₂O/0.1% TFA]:[99.9% H₂O/0.1% TFA]) to 1:1 ([95% CH₃CN/4.9% H₂O/0.1% TFA]: [99.9% H₂O/0.1% TFA]) over 30 min at a flow rate of 20 mL/min, monitoring at 420 nm. The product was collected at 12 min. The solution containing the product was frozen, and the solvents removed via the use of a lyophilizer providing 134 as a red solid (9.1 mg, 10.2 μmol, 39%): R_{f}: 0.28 (1:1 acetonitrile-water, reverse phase); ¹H NMR (500 MHz, CD₃OD 6) 1.40 (m, 2H), 1.64 (m, 2H), 1.75 (m, 2H), 2.30 (m, 2H), 2.32 (s, 3H), 2.71 (sept, J=7.5 Hz, 2H), 2.79 (t, J=7.5 Hz, 2H), 2.86 (s, 3H), 3.01 (m, 1H), 3.14 (s, 9H), 3.16-3.42 (m, 9H), 3.49-3.56 (m, 1H), 3.64-3.70 (m, 1H), 4.67 (m, 1H), 6.30 (s, 1H), 6.77 (s, 1H), 7.17 (dd, J=5, 3.5 Hz, 1H), 7.51 (s, 1H), 7.60 (d, J=5 Hz, 1H), 8.05 (d, J=3.5 Hz, 1H), 8.18 (m, 1H) (NH), 8.41 (m, 1H) (NH); ¹³C NMR (125 MHz, CD₃OD) δ 11.58, 23.22, 24.75, 25.12, 25.54, 25.68, 27.15, 34.68, 35.43, 35.61, 40.84, 40.96, 52.30, 53.06, 53.21, 53.82, 57.24, 57.33, 57.68, 57.84, 67.39, 119.38, 120.20, 122.40, 129.80, 130.26, 131.96, 135.45, 135.74, 135.91, 144.67, 144.93, 149.88, 162.38, 173.69, 174.29, 177.32; HRMS [M] calcd for C₃₅H₅₂BF₂N₆O₇S₂⁺ 781.3401. found 781.3432.

6-(4,4-Difluoro-1-methyl-5-(2-thienyl)-7-(trimethylammonium trifluoroacetate)-propyl-4-bora-3a,4a,diaza-s-indacene-3-(N-methyl-N-(2-((R)-2-propionamido-3-sulfono-propionamido)ethyl)amino)hexanoic acid, succinimidyl ester (135): Anhydrous DMF (4 mL) was added to a flask containing acid 134 (9.1 mg, 10.2 μmol), NHS (11.5 mg, 0.100 mmol) and DCC (17.9 mg, 0.0868 mmol) under argon at ambient temperature. The mixture was stirred at ambient temperature for 15 h before the solvent was removed via the use of a lyophilizer. The crude product was purified via reverse phase HPLC using a gradient of 3:7 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 420 nm. The product was collected at 13 min. The solution containing the product was frozen, and the solvents removed via the use of a lyophilizer providing 135 as a red solid (6.5 mg, 6.55 μmol, 64%): R_{f}: 0.36 (3:1 acetonitrile-water); ¹H NMR (500 MHz, CD₃OD) δ 1.49 (m, 2H), 1.79 (m, 4H), 2.19 (m, 2H), 2.35 (s, 3H), 2.64-2.75 (m, 4H), 2.81-2.85 (m, 6H), 2.87 (s, 4H), 3.02 (m, 1H), 3.15 (m, 9H), 3.19 (m, 2H), 3.25 (m, 4H), 3.42 (m, 2H), 3.53-3.69 (m, 2H), 4.66 (bs, 1H), 6.31 (s, 1H), 6.80 (s, 1H), 7.17 (dd, J=5, 4 Hz, 1H), 7.55 (s, 1H), 7.61 (d, J=5 Hz, 1H), 8.06 (d, J=4 Hz, 1H), 8.17 (dd, J=21, 7 Hz, 1H) (NH), 8.42 (bs, 1H) (NH); ¹³C NMR (125 MHz, CD₃OD) δ 11.60, 23.23, 24.49, 25.14, 25.68, 26.66, 31.40, 35.43, 35.59, 40.96, 41.02, 52.33, 53.06, 53.20, 53.84, 57.33, 57.57, 57.66, 67.39, 119.40, 120.22, 122.43, 131.96, 135.45, 135.74, 135.92, 144.68, 144.98, 149.87, 162.43, 170.34, 172.06, 173.69, 174.31; HRMS [M] calcd for C₃₉H₅₅BF₂N₇O₉S₂⁺ 878.3565. found 878.3566.

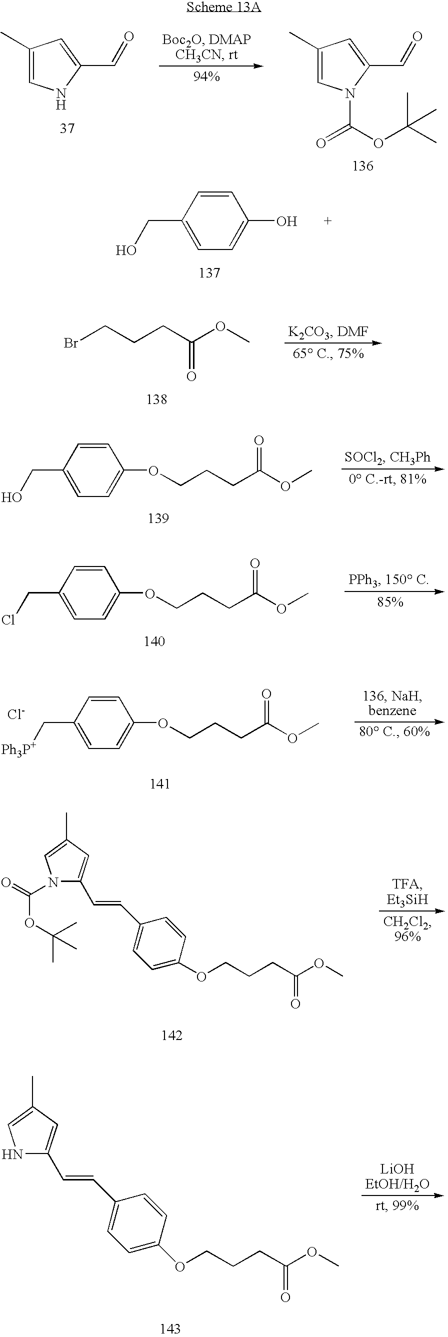

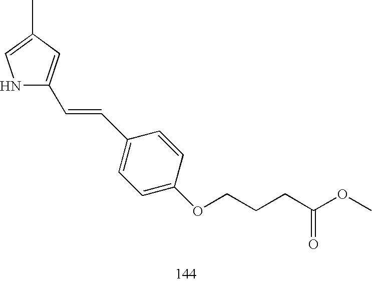

tert-Butyl 2-formyl-4-methyl-1H-pyrrole-1-carboxylate (136): To a stirred solution of 4-methyl-2-formylpyrrole (1.66 g, 15.2 mmol) in anhydrous acetonitrile (33 mL) under argon at room temperature was added DMAP (204 mg, 1.66 mmol), followed by Boc₂O (3.26 g, 14.9 mmol). The mixture was stirred at room temperature overnight, followed by partitioning between 0.1 M HCl (70.5 mL) and ether (150 mL). The organic layer was washed with 36 mL portions of 0.1 M HCl (5×), H₂O (1×), sat'd NaHCO₃ (3×), and brine (3×). The organic layer was dried over MgSO₄, followed by filtration and removal of the solvent in vacuo. The crude product was then purified by flash chromatography on silicagel (10% EtOAc; 90% hexanes) to yield a clear liquid (2.93 g, 14.0 mmol, 94%): FTIR: 1094(s), 1140(s), 1163(s), 1274(s), 1345(s), 1424(s), 1469(s), 1669(s), 1744(s), 2904(s), 2932(s), 2980(s); ¹H NMR (500 MHz, CD₃OD) δ 1.63 (s, 9H), 2.07 (s, 3H), 6.99 (s, 1H), 7.29 (s, 1H), 10.19 (s, 1H); ¹³C NMR (500 MHz, CD₃OD) δ 11.46, 28.25, 86.82, 123.74, 123.90, 127.08, 136.04, 149.90, 183.89; R_{f}: 0.33 (5% ethyl acetate in hexanes); HRMS-ES (m/z): [M+Na] calcd for C₁₁H₁₅NO₃Na⁺ 232.0950. found 232.0948.

Methyl 4-(4-(hydroxymethyl)phenoxy)butanoate (139): To a flask containing 4-hydroxy-benzyl alcohol (137) (5.46 g, 30.2 mmol) and K₂CO₃ (30.95 g, 224 mmol) under Argon was added a solution of methyl 4-bromobutanoate (138) (3.67 g, 29.6 mmol) in dry DMF (72 mL). The mixture was stirred at 65° C. for 16 hr. before being cooled and filtered. The filtered solution was poured into EtOAc (300 mL) and washed with H₂O (6×150 mL). The organic layer was dried over Na₂SO₄, followed by filtration and removal of the solvent in vacuo to yield a clear yellow liquid (4.98 g, 22.2 mmol, 75%): FTIR: 1006(s), 1042(s), 1173(s), 1246(s), 1513(s), 1612(s), 1731(s), 2875(s), 2952(s), 3430(br); ¹H NMR (500 MHz, CD₃OD) δ 2.06 (p, J=11, 2H), 2.52 (t, J=12, 2H), 3.67 (s, 3H), 4.00 (t, J=10, 2H), 4.51 (s, 2H), 6.87 (d, J=14, 2H), 7.25 (d, J=14, 2H); ¹³C NMR (500 MHz, CD₃OD) δ 25.84, 31.47, 52.22, 64.95, 67.94, 115.42, 129.69, 134.90, 159.65, 175.43; HRMS-ES (m/z): [M+Na] calcd for C₁₂H₁₆O₄Na⁺ 247.0946. found 247.0937.

Methyl 4-(4-(chloromethyl)phenoxy)butanoate (140): A stirred solution of methyl 4-(4-(hydroxymethyl)phenoxy)butanoate (7.52 g, 33.5 mmol) in dry toluene (37.2 mL) under Argon was cooled to 0° C., followed by dropwise addition of SOCl₂ (4.9 mL, 41.0 mmol). The mixture was stirred at room temperature for 2 days before the solvent was removed in vacuo. The crude product was purified by flash chromatography on silicagel (20% EtOAc; 80% hexanes) to yield a clear liquid (6.61 g, 27.3 mmol, 81%): FTIR: 1174(s), 1245(s), 1435(s), 1514(s), 1611(s), 1736(s), 2952(s); ¹H NMR (500 MHz, CD₃OD) δ 2.06 (p, J=6.5, 2H), 2.51 (t, J=7, 2H), 3.67 (s, 3H), 4.00 (t, J=6, 2H), 4.58 (s, 2H), 6.87 (d, J=9, 2H), 7.30 (d, J=8.5, 2H); ¹³C NMR (500 MHz, CD₃OD) δ 25.98, 31.58, 47.04, 52.20, 68.14, 75.38, 115.56, 115.79, 130.72, 131.30, 131.71, 160.21; R_{f}: 0.38 (10% ethyl acetate in hexanes); HRMS-ES (m/z): [M+Na] calcd for C₁₂H₁₅ClO₃Na⁺ 265.0607. found 265.0619.

4-(Methyl-4-butanoate)benzyl)triphenylphosphonium chloride (141): A flask containing methyl 4-(4-(chloromethyl)phenoxy)butanoate (3.338 g, 13.75 mmol) and triphenylphosphine (3.605 g, 13.74 mmol) were stirred under argon and stirred at 160° C. overnight. The crude product was purified by flash chromatography on silicagel (5% MeOH; 95% CH₂Cl₂) to yield a clear, viscous liquid (5.55 g, 11.0 mmol, 80%): ¹H NMR (500 MHz, CD₃OD) δ 2.02 (p, J=7, 2H), 2.48 (t, J=7, 2H), 3.63 (s, 3H), 3.96 (t, J=6, 2H), 5.01 (d, J=14, 2H), 6.77 (d, J=8.5, 2H), 7.00 (dd, J=8.5, J=2.5, 2H), 7.71-7.78 (m, 12H), 7.89-7.92 (m, 3H); ¹³C NMR (500 MHz, CD₃OD) δ 25.68, 31.41, 52.27, 68.16, 116.12, 118.95, 119.63, 131.36, 131.46, 133.54, 133.58, 135.37, 135.44, 136.40, 160.53, 175.19; HRMS-ES (m/z): [M] calcd for C₃₀H₃₀O₃P⁺ 469.1932. found 469.1935.

tert-Butyl-2-(4-(3-(methoxycarbonyl)propoxy)styryl)-4-methyl-1H-pyrrole-1-carboxylate (142): To a stirred suspension of (4-(methyl-4-butanoate)benzyl)triphenylphosphonium chloride (500 mg, 0.99 mmol) in anhydrous benzene (1.1 mL) under argon at 80° C. was added NaH (23.2 mg, 0.97 mmol). The suspension was stirred at 80° C. for 30 min before a solution of tert-butyl 2-formyl-4-methyl-1H-pyrrole-1 carboxylate (209 mg, 1.00 mmol) in anhydrous benzene (1.1 mL) was added. The mixture was refluxed for 4 hours before being cooled. The crude material was dissolved in ether and absorbed onto silicagel. The crude product was purified via flash chromatography on silicagel (5% EtOAc; 95% hexanes) to yield a clear yellow oil which was a 1:1 mixture of isomers which were not resolved (232 mg, 0.58 mmol, 60%): HRMS-ES (m/z): [M+Na] calcd for C₂₃H₂₉NO₅Na⁺ 422.1943. found 422.1951.

Methyl-4-(4-((E)-2-(4-methyl-1H-pyrrol-2-yl)vinyl)phenoxy)butanoate (143): To a stirred solution of tert-Butyl-2-(4-(3-(methoxycarbonyl)propoxy)styryl)-4-methyl-1H-pyrrole-1-carboxylate (184 mg, 0.461 mmol) in dry DCM (2.5 mL) under argon at room temperature was added Et₃SiH (0.08 mL, 0.500 mmol) and trifluoroacetic acid (2.5 mL). The mixture was stirred at room temperature for 45 min followed by quenching with H₂O. The mixture was basified with 1 M NaOH and extracted with DCM. The organic layer was washed with 1 M HCl and brine, followed by drying the organic layer with Na₂SO₄. Filtration and removal of the solvent in vacuo yielded a purple solid (132 mg, 0.441 mmol, 96%): FTIR (CH₂Cl₂): 1173(s), 1249(s), 1512(s), 1722(s); ¹H NMR (500 MHz, CDCl₃) δ 2.10-2.15 (m, 5H), 2.55 (t, J=7, 2H), 3.71 (s, 3H), 4.02 (t, J=6, 2H), 6.17 (s, 1H), 6.55 (s, 1H), 6.59 (d, J=16.5, 1H), 6.78 (d, J=16.5, 1H), 6.85 (d, J=7.5, 2H), 7.33 (d, J=7.5, 2H), 8.09 (bs, 1H); ¹³C NMR (500 MHz, CDCl₃) δ 12.04, 24.84, 30.75, 51.83, 66.95, 109.99, 114.91, 116.86, 117.44, 120.64, 122.90, 127.11, 130.76, 131.22, 158.16, 173.88; HRMS-ES (m/z): [M+Na] calcd for C₁₈H₂₁NO₃Na⁺ 322.1419. found 322.1428.

4-(4-((E)-2-(4-methyl-1H-pyrrol-2-yl)vinyl)phenoxy)butanoic acid (144): To a flask containing methyl-4-(4-((E)-2-(4-methyl-1H-pyrrol-2-yl)vinyl)phenoxy)butanoate (57 mg, 0.190 mmol) in THF under normal atmosphere was added a solution of aqueous LiOH (23 mg, 0.960 mmol in 1.6 mL H₂O). The mixture was stirred at room temperature until the completion of the reaction was indicated by TLC. The mixture was acidified with 1 M HCl and extracted with EtOAc. The organic layer was dried with Na₂SO₄, followed by filtration and removal of the solvent in vacuo to yield a purple solid (54 mg, 0.189 mmol, 99%): ¹H NMR (500 MHz, CD₃OD) δ 1.99-2.06 (m, 5H), 2.46 (t, J=7.5, 2H), 3.99 (t, J=6, 2H), 6.00 (s, 1H), 6.46 (s, 1H), 6.63 (d, J=16.5, 1H), 6.75 (d, J=16, 1H), 6.83 (d, J=9, 2H), 7.31 (d, J=8.5, 2H); ¹³C NMR (500 MHz, CD₃OD) δ 12.09, 26.08, 31.63, 68.23, 110.50, 115.90, 118.07, 119.01, 120.52, 123.19, 127.88, 132.52, 132.69, 159.40, 177.26; R_{f}: 0.33 (5% methanol in dichloromethane); HRMS-ES (m/z): [M+H] calcd for C₁₇H₂₀NO₃⁺ 286.1438. found 286.1409.

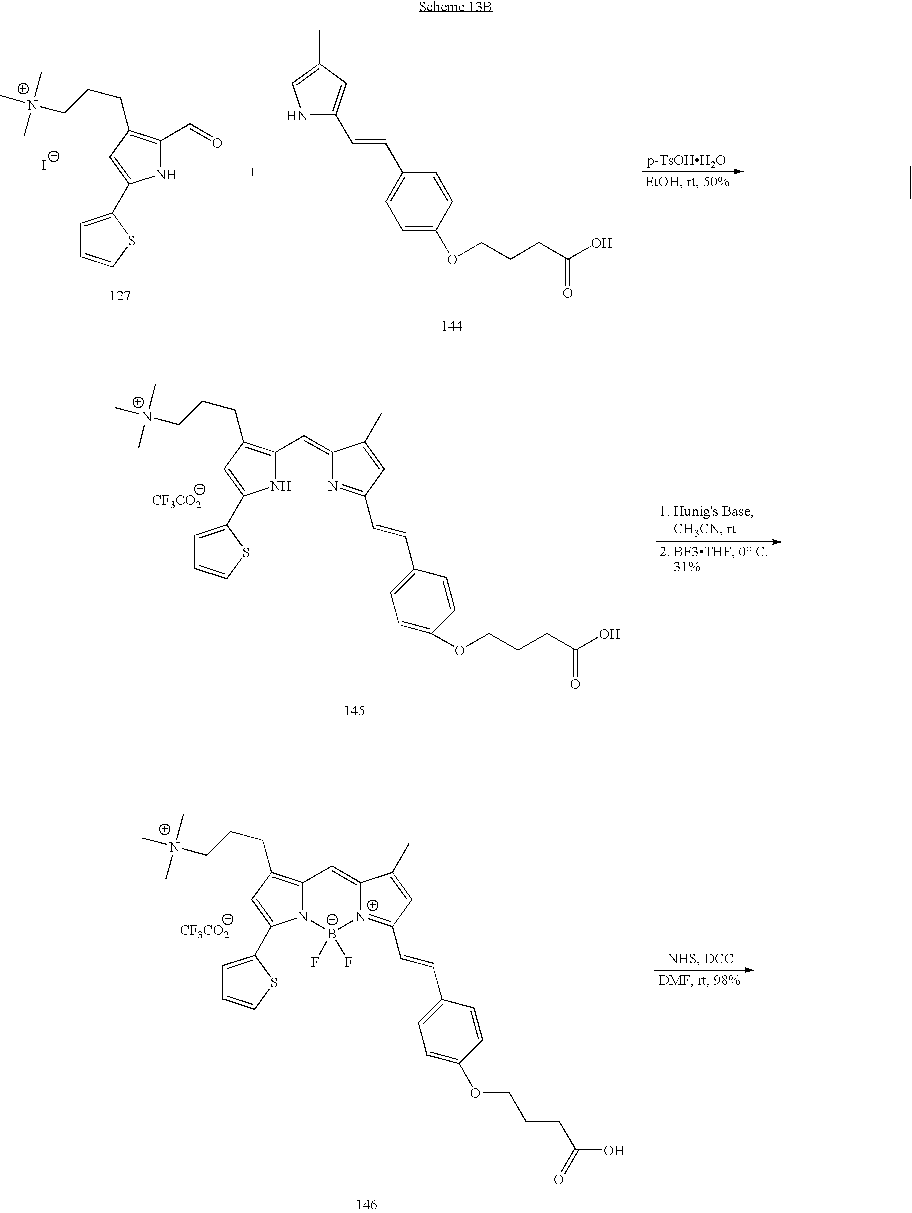

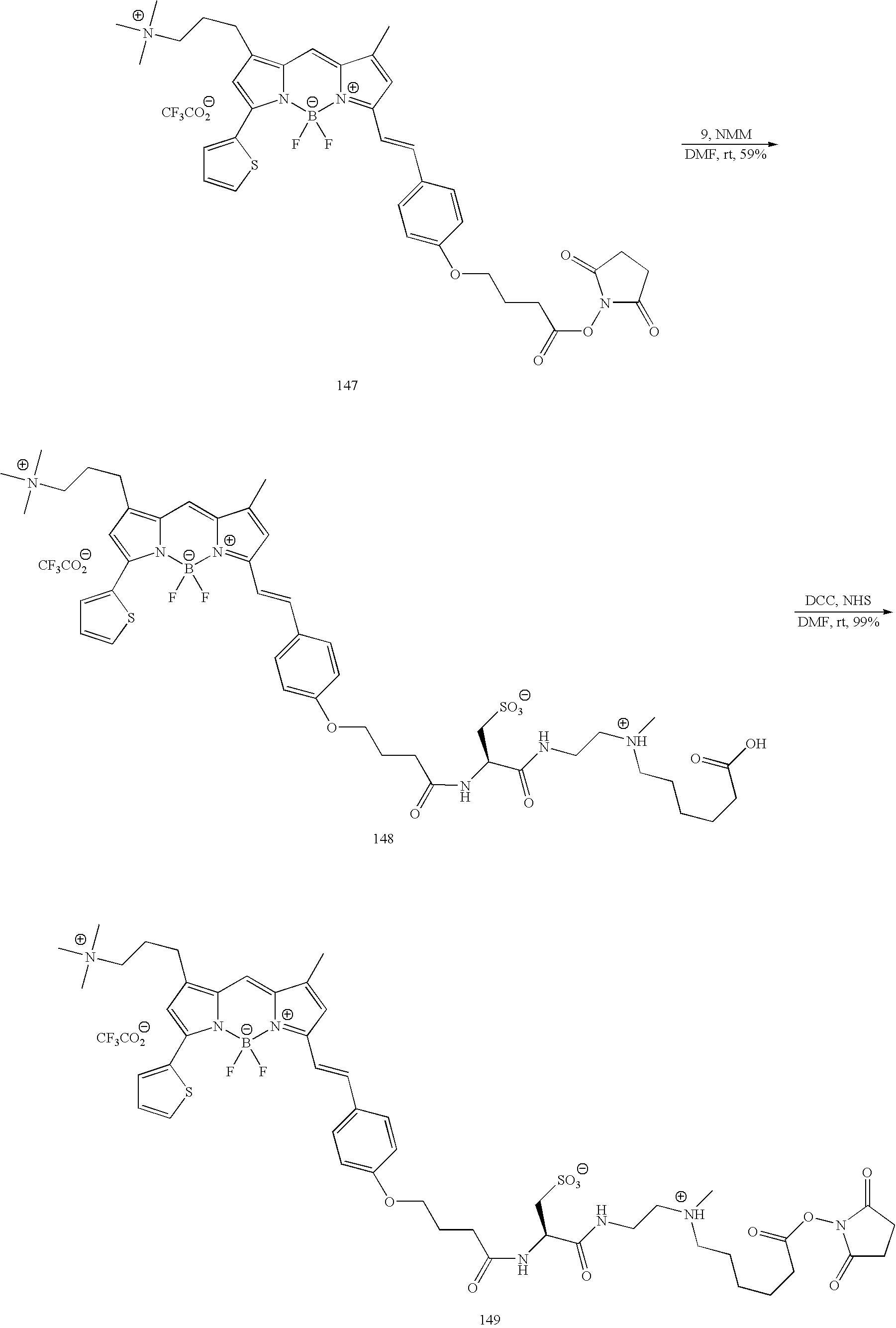

5-(2-thienyl)-3-(trimethylammonium trifluoroacetate)-propyl-3′-methyl-5′-(4-(4-(E)-vinylphenoxy)butanoic acid)dipyrromethene (145): To a stirred suspension of trimethyl-(3-(2-formyl-5-(2-thienyl)-1H-3-pyrrolyl)-propyl)-ammonium iodide 127 (37.5 mg, 92.8 μmol) and 4-(4-((E)-2-(4-methyl-1H-pyrrol-2-yl)vinyl)phenoxy)butanoic acid 144 (26.5 mg, 84.6 μmol) in ethanol (14 mL) was added p-TsOH monohydrate (17.6 mg, 92.5 μmol). The mixture was stirred at room temperature for 30 minutes before removal of the solvent in vacuo. The crude was purified via HPLC to yield a dark purple solid (28 mg, 42.6 μmol, 50%): ¹H NMR (500 MHz, CD₃CN) δ 1.96-2.05 (m, 4H), 2.43 (s, 3H), 2.47 (t, J=7, 2H), 2.64 (t, J=7.5, 2H), 3.04 (s, 9H), 3.26-3.30 (m, 2H), 3.95 (t, J=6.5, 2H), 6.56 (s, 1H), 6.58 (s, 1H), 6.75 (d, J=8.5, 2H), 6.77 (s, 1H), 6.82 (d, J=16, 1H), 7.07 (dd, J=4, J=3, 1H), 7.23 (d, J=16.5, 1H), 7.29 (d, J=8.5, 2H), 7.55 (d, J=4, 1H), 7.79 (d, J=3, 1H); ¹³C NMR (500 MHz, CD₃CN) δ 12.76, 23.66, 24.26, 25.64, 31.35, 54.30, 67.04, 68.37, 114.43, 115.36, 116.23, 117.88, 129.54, 130.20, 130.36, 130.84, 131.58, 131.95, 133.31, 142.08, 145.67, 147.62, 148.97, 156.29, 162.22; HRMS-ES (m/z): [M] calcd for C₃₂H₃₈N₃O₃S⁺ 544.2628. found 544.2624.

4-((4,4-difluoro-1-methyl-5-(2-thienyl)-7-(3-trimethylammonium trifluoroacetate)-propyl-4-bora-3a,4a,diaza-s-indacene-yl)styryloxy)butanoic acid (146): To a stirred solution of 5-(2-thienyl)-3-(trimethylammonium trifluoroacetate)-propyl-3′-methyl-5′-(4-(4-(E)-vinylphenoxy)butanoic acid)dipyrromethene (61 mg, 92.7 μmol) in anhydrous acetonitrile (15 mL) under argon at room temperature was added diisopropylethylamine (645 μL, 3.70 mmol), and the resultant mixture was stirred at room temperature for 5 minutes. The mixture was then cooled to 0° C. and BF₃.THF (82 μL, 0.743 mmol) was added, and the mixture was stirred at 0° C. for 30 minutes before the solvent was removed in vacuo at 0° C. The crude was purified via HPLC to yield a dark blue solid (20 mg, 28.3 μmol, 31%): ¹H NMR (500 MHz, CD₃CN) δ 2.03 (p, J=7, 2H), 2.07-2.13 (m, 2H), 2.35 (s, 3H), 2.46 (t, J=7.5, 2H), 2.76 (t, J=7.5, 2H), 3.02 (s, 9H), 3.27-3.31 (m, 2H), 4.06 (t, J=6.5, 2H), 6.76 (s, 1H), 6.90 (s, 1H), 6.98 (d, J=9, 2H), 7.22 (dd, J=4.5, J=3.5, 1H), 7.38 (s, 1H), 7.42 (d, J=16.5, 1H), 7.51 (d, J=16, 1H), 7.56 (d, J=8.5, 2H), 7.60 (dd, J=5, J=0.5, 1H), 8.05 (d, J=3, 1H); ¹³C NMR (500 MHz, CD₃OD) δ 11.49, 23.26, 25.21, 25.97, 31.52, 53.82, 67.50, 68.41, 116.31, 117.61, 118.31, 118.81, 119.28, 129.36, 129.69, 130.55, 130.66, 131.16, 135.62, 135.97, 138.04, 140.15, 142.34, 144.44, 148.00, 158.58, 162.11, 177.08; HRMS-ES (m/z): [M] calcd for C₃₂H₃₇BF₂N₃O₃S⁺ 592.2617. found 592.2609.

4-((4,4-difluoro-1-methyl-5-(2-thienyl)-7-(3-trimethylammonium trifluoroacetate)-propyl-4-bora-3a,4a,diaza-s-indacene-yl)styryloxy)butanoic acid, succinimidyl ester (147): A solution of DCC (78 mg, 0.378 mmol) in anhydrous DMF was added to a round bottom flask, equipped with a stir bar, containing 4-((4,4-difluoro-1-methyl-5-(2-thienyl)-7-(3-trimethylammonium trifluoroacetate)-propyl-4-bora-3a,4a,diaza-s-indacene-yl)styryloxy)butanoic acid 146 (29.6 mg, 42.0 μmol) and NHS (48.3 mg, 0.420 mmol) under argon. The mixture was stirred at room temperature for 5.5 hours before the solvent was removed in vacuo. The crude was purified via HPLC to yield a dark blue solid (33 mg, 41.1 mmol, 98%): ¹H NMR (500 MHz, CD₃CN) δ 2.14-2.08 (m, 2H), 2.18 (p, J=7, 2H), 2.37 (s, 3H), 2.76-2.79 (m, 6H), 2.83 (t, J=7.5, 2H), 3.03 (s, 9H), 3.28-3.32 (m, 2H), 4.13 (t, J=6.5, 2H), 6.77 (s, 1H), 6.92 (s, 1H), 7.01 (d, J=8.5, 2H), 7.22 (dd, J=5.5, J=3.5, 1H), 7.40 (s, 1H), 7.44 (d, J=16.5, 1H), 7.53 (d, J=16.5, 1H), 7.58 (d, J=8.5, 2H), 7.60 (d, J=5.5, 1H), 8.06 (d, J=3.5, 1H); ¹³C NMR (500 MHz, CD₃CN) δ 11.72, 22.89, 24.75, 25.27, 26.52, 28.38, 54.06, 66.97, 67.48, 116.30, 117.08, 118.16, 119.02, 120.09, 129.82, 129.93, 130.21, 130.34, 131.00, 135.33, 135.54, 137.47, 139.86, 142.78, 144.46, 147.39, 157.71, 161.43, 170.01, 171.25; HRMS-ES (m/z): [M] calcd for C₃₆H₄₀BF₂N₄O₅S⁺ 689.2781. found 689.2756.

6-(((4,4-difluoro-1-methyl-5-(2-thienyl)-7-(3-trimethylammonium)-propyl-4-bora-3a,4a,diaza-s-indacene-3-yl)styryloxy)N-methyl-N-(2-(2-butanamido-3-sulfonate-propionamido)ethyl)amino))hexanoic acid, hydrotrifluoroacetate (148): To a stirred mixture of 4-((4,4-difluoro-1-methyl-5-(2-thienyl)-7-(3-trimethylammonium trifluoroacetate)-propyl-4-bora-3a,4a,diaza-s-indacene-yl)styryloxy)butanoic acid, succinimidyl ester 147 (33 mg, 41.1 μmol) and 6-(N-methyl-N-(2-(2-amino-3-sulfonate-propionamido)ethyl)amino)hexanoic acid, hydroacetate 9 (52 mg, 0.130 mmol) in anhydrous DMF under argon at room temperature was added NMM (91 μL, 0.847 mmol). The mixture was stirred at room temperature for 3 hours before the solvent was removed in vacuo. The crude was purified via HPLC to yield a dark blue solid (25 mg, 24.3 μmol 59%): ¹H NMR (500 MHz, CD₃OD) δ 1.36-1.41 (m, 2H), 1.64 (p, J=7.5, 2H), 1.74 (p, J=8, 2H), 2.03 (s, 3H), 2.06-2.10 (m, 2H), 2.10-2.16 (m, 2H), 2.29-2.32 (m, 5H), 2.46 (bs, 2H), 2.76 (t, J=7.5, 2H), 2.85 (s, 3H), 2.99 (bs, 1H), 3.11 (s, 9H), 3.19-3.30 (m, 2H), 3.36-3.39 (m, 2H), 3.44-3.47 (m, 1H), 3.58 (bs, 1H), 3.71 (bs, 1H); 4.02 (t, J=6, 2H), 4.69 (dd, J=7.5, J=4.5, 1H), 6.74 (s, 1H), 6.86 (s, 1H), 6.93 (d, J=9, 2H), 7.19 (dd, J=5.5, J=3.5, 1H), 7.33 (s, 1H), 7.42 (d, J=16.5, 1H), 7.46 (d, J=16.5, 1H), 7.52 (d, J=8.5, 2H), 7.58 (d, J=5.5, 1H), 8.08 (d, J=3.5, 1H); ¹³C NMR (500 MHz, CD₃OD) δ 11.57, 23.28, 24.74, 25.10, 25.54, 26.28, 27.14, 33.54, 34.68, 35.45, 40.91, 52.19, 53.25, 53.81, 57.29, 57.73, 67.47, 68.52, 89.43, 102.09, 116.38, 117.57, 118.37, 118.81, 119.31, 129.51, 129.79, 130.58, 131.20, 135.63, 136.00, 138.00, 140.12, 142.44, 144.44, 147.89, 158.40, 162.02, 173.73, 175.22, 177.28; HRMS-ES (m/z): [M] calcd for C₄₄H₆₀BF₂N₆O₈S₂⁺ 913.3977. found 913.3991.

6-(((4,4-difluoro-1-methyl-5-(2-thienyl)-7-(3-trimethylammonium)-propyl-4-bora-3a,4a,diaza-s-indacene-3-yl)styryloxy)N-methyl-N-(2-(2-butanamido-3-sulfonate-propionamido)ethyl)amino)hexanoic acid, succinimidyl ester, hydrotrifluoroacetate (149): A solution of DCC (45 mg, 0.218 mmol) in anhydrous DMF (4.4 mL) was added to a round bottom flask, equipped with a stir bar, containing 6-(((4,4-difluoro-1-methyl-5-(2-thienyl)-7-(3-trimethylammonium trifluoroacetate)-propyl-4-bora-3a,4a,diaza-s-indacene-3-yl)styryloxy)N-methyl-N-(2-(2-butanamido-3-sulfonate-propionamido)ethyl)amino)hexanoic acid, hydroacetate 148 (25 mg, 24.3 μmol) and NHS (28 mg, 0.243 mmol) under argon. The mixture was stirred at room temperature for 3 hours before the solvent was removed in vacuo. The crude was then purified via HPLC to yield a dark blue solid (27 mg, 24 μmol, 99%): ¹H NMR (500 MHz, CD₃CN) δ 1.39-1.47 (m, 2H), 1.69-1.76 (m, 4H), 2.03-2.13 (m, 4H), 2.34 (s, 3H), 2.41-2.46 (m, 2H), 2.64 (t, J=7, 2H), 2.75-2.80 (m, 11H), 3.02 (s, 9H), 3.23-3.40 (m, 7H), 3.66-3.82 (m, 1H), 4.05-4.06 (m, 2H), 4.61 (bs, 1H), 6.76 (s, 1H), 6.88 (s, 1H), 6.97 (dd, J=8.5, J=1.5, 2H), 7.22 (dd, J=4.5, J=3.5, 1H), 7.38 (s, 1H), 7.41 (d, J=16.5, 1H), 7.49 (d, J=16, 1H), 7.55 (dd, J=8.5, J=1.5, 2H), 7.60 (d, J=4.5, 1H), 7.90 (NH) (dd, J=49.5, J=7, 1H), 8.05 (d, J=3.5, 1H), 8.88-8.93 (NH) (m, 1H); ¹³C NMR (500 MHz, CD3CN) δ 11.76, 22.92, 23.88, 24.75, 24.82, 25.80, 26.25, 26.52, 31.23, 33.25, 33.32, 34.84, 35.01, 41.11, 41.30, 51.80, 52.82, 52.97, 54.06, 56.47, 56.56, 56.75, 57.19, 66.96, 68.47, 116.25, 116.92, 118.19, 118.94, 119.95, 129.85, 129.95, 130.37, 130.96, 135.27, 135.57, 137.49, 140.02, 142.62, 144.41, 147.22, 157.83, 161.62, 170.14, 171.28, 172.80, 172.97, 173.44; HRMS-ES (m/z): [M] calcd for C₄₈H₆₃BF₂N₇O₁₀S₂⁺ 1010.4142. found 1010.4198.

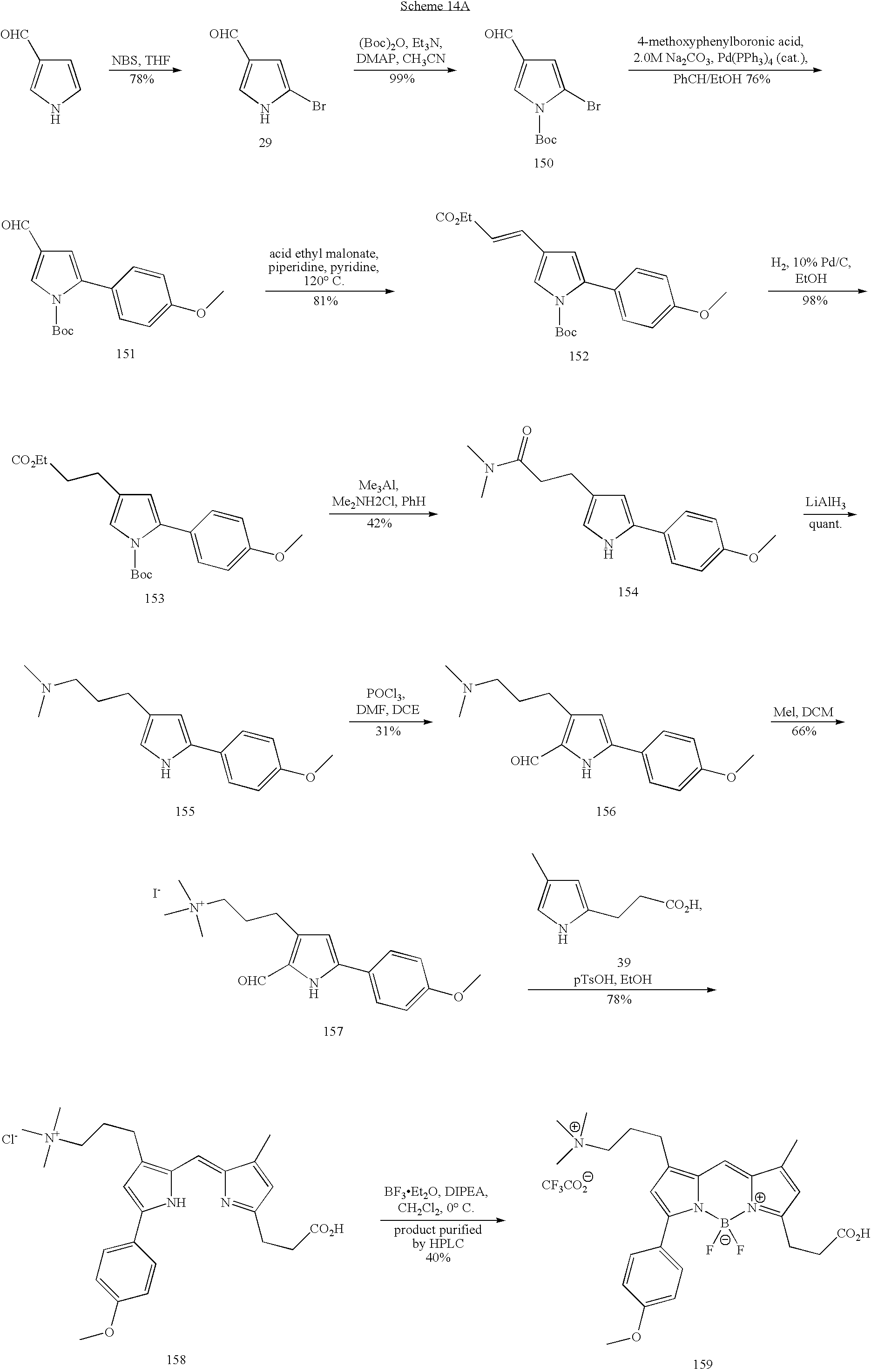

### 5-bromo-1H-pyrrole-3-carbaldehyde (29)

To a round-bottom flask containing 1H-pyrrole-3-carbaldehyde (2.84 g, 29.9 mmol, 1.0 eq) under argon was added 150 mL dry tetrahydrofuran. The reaction vessel was placed in a −78° C. isopropanol bath and the solution was allowed to stir for 10 min before adding freshly recrystallized N-bromosuccinimide (5.32 g, 29.9 mmol, 1.0 eq). The bath temperature was raised to −50° C. and the reaction was stirred at this temperature for 15 hrs. Reaction progress was monitored by H NMR. Upon completion, the solvent was removed in vacuo and the crude residue purified by column chromatography on Davisil (5% isopropanol, 95% hexanes) to yield 29 as a white powder (4.10 g, 23.6 mmol, 78%). TLC Rf=0.11 (5% isopropanol, 95% hexanes). 1H NMR (500 MHz, acetone-d6): δ 6.58 (d, 1H), 7.67 (d, 1H), 9.71 (s, 1H). 13C NMR (125 MHz, acetone-d6): δ 102.83, 109.10, 129.01, 129.90, 184.75. HRMS (ESI-TOF) MH+ of C5H4BrNO+ calculated (m/z)=172.9476. found (m/z)=172.9481.

### tert-Butyl 2-bromo-4-formyl-1H-pyrrole-1-carboxylate (150)

A round-bottom flask containing 5-bromo-1H-pyrrole-3-carbaldehyde 29 (1.080 g, 6.21 mmol, 1.0 eq) and dimethylaminopyridine (0.076 g, 0.621 mmol, 0.1 eq) was flushed with argon and the solids were dissolved with 15.5 mL anhydrous acetonitrile. Freshly distilled triethylamine (0.866 mL, 6.21 mmol, 1.0 eq) and di-tert-butyl pyrocarbonate (1.360 g, 6.21 mmol, 1.0 eq) were added to the reaction flask. The solution was stirred under argon overnight and poured into 100 mL diethyl ether. The ether layer was washed with 3×50 mL of 1 M potassium bisulfate, 1×50 mL of DI water, and 1×50 mL saturated aqueous sodium bicarbonate. The organic layer was dried over sodium sulfate the solvent removed in vacuo. The crude solid was purified by column chromatography on Davisil (5% ethyl acetate, 95% hexanes). Removal of the solvent in vacuo at 50° C. provided analytically pure 150 as a white powder. A crystalline product was obtained dissolving the solid in diethyl ether and diluting with hexanes, followed by removal of the solvent in vacuo at room temperature. (1.40 g, 5.1 mmol, 99% yield). TLC Rf=0.33 (20% ethyl acetate, 80% hexanes). 1H NMR (500 MHz, chloroform-d3): δ 1.654 (s, 9H), 6.756 (s, 1H), 7.927 (s, 1H), 9.752 (s, 1H). 13C NMR (500 MHz, chloroform-d3): δ 55.156, 101.663, 114.316, 118.063, 124.268, 125.645, 127.278, 134.177, 158.440, 185.131. HRMS (ESI-TOF) MH+ of C10H12BrNO3 calculated (m/z)=273.0001. found (m/z)=273.0072.

### tert-Butyl 4-formyl-2-(4-methoxyphenyl)-1H-pyrrole-1-carboxylate (151)

A Schlenk tube was charged with tert-butyl 2-bromo-4-formyl-1H-pyrrole-1-carboxylate (1.000 g, 3.66 mmol, 1.0 eq), 4-methoxyphenylboronic acid (0.557 g, 3.66 mmol, 1.0 eq), and tetrakis(triphenylphosphine) palladium (0) (0.212 g, 0.18 mmol, 0.05 eq). The vessel was evacuated and refilled with argon thrice. Freshly distilled toluene (21 mL), degassed 95% aqueous ethanol (,2.5 mL), and degassed 2.0 M aqueous sodium carbonate (3.65 mL, 7.33 mmol, 2 eq) were added via syringe. The mixture was placed in an oil bath (preheated to 85° C.) and stirred under argon for 14 hours. The mixture was then cooled and partitioned with deionized water. The resulting mixture was transferred to a separatory funnel and extracted twice into dichloromethane. The organic fractions were combined and dried over sodium sulfate. The solvent was removed in vacuo. The crude residue was purified by column chromatography (5% ethyl acetate, 95% hexanes) on Davisil to give a solid product (0.8057 g, 2.79 mmol, 76% yield). TLC Rf=0.24 (20% ethyl acetate, 80% hexanes). 1H NMR (500 MHz, acetone-d6): δ 1.403 (s, 9H), 3.840 (s, 3H), 6.476 (s, 1H), 6.976 (d, 2H, J=9 Hz), 7.338 (d, 2H, J=8.5 Hz), 8.119 (s, 1H), 9.861 (s, 1H). 13C NMR (500 MHz, chloroform-d6): δ 27.478, 55.210, 85.229, 110.353, 33 113.202, 125.263, 127.003, 130.422, 130.692, 137.088, 148.383, 159.476, 185.526. HRMS (ESI-TOF) MH+ of C17H19NO4+ calculated (m/z)=301.1314. found (m/z)=301.1386.

### (E)-tert-Butyl-4-(3-ethoxy-3-oxoprop-1-enyl)-2-(4-methoxyphenyl)-1H-pyrrole-1-carboxylate (152)

To a round-bottom flask containing a stirred solution of tert-butyl 4-formyl-2-(4-methoxyphenyl)-1H-pyrrole-1-carboxylate (0.3602 g, 1.195 mmol, 1.0 eq) and piperidine (24 μL, 0.239 mmol, 0.2 eq) in anhydrous pyridine (1.06 mL, 13.15 mmol, 11.0 eq) under argon was added mono-ethyl malonate (0.85 mL, 7.172 mmol, 6 eq). The reaction flask was placed in an oil bath (preheated to 120° C.) for six hours. After bringing the reaction mixture to room temperature, the mixture was poured into a separatory funnel containing 50 mL deionized water and was extracted exhaustively into diethyl ether. The organic fractions were combined and washed successively with 50 mL of 0.1 M aqueous potassium bisulfate, 50 mL of 0.1 M aqueous sodium bicarbonate, 50 mL deionized water, and 50 mL brine. The organic phase was dried over sodium sulfate. The solvent was removed in vacuo, giving a viscous orange residue. The crude residue was purified by recrystallization with methanol after triturating with hexanes, yielding an orange solid. (0.4241 g, 1.14 mmol, 95.5% yield). TLC Rf=0.42 (20% ethyl acetate, 80% hexanes). 1H NMR (500 MHz, chloroform-d3): δ 1.291 (t, 3H, J=7.5 Hz), 1.361 (s, 9H), 3.798 (s, 3H), 4.208 (q, 2H, J=7 Hz), 6.127 (d, 1H, J=16 Hz), 6.309 (s, 1H), 6.874 (d, 2H, J=8.5 Hz), 7.239 (d, 2H, J=8.5 Hz), 7.494 (s, 1H), 7.549 (d, 1H, J=15.5 Hz). 13C NMR (500 MHz, chloroform-d3): δ 14.468, 27.734, 55.373, 60.318, 84.395, 111.236, 113.249, 34 116.611, 122.037, 124.660, 126.005, 130.516, 136.859, 137.266, 148.828, 159.412, 167.478. HRMS (ESI-TOF) MH+ of C21H25NO5+ calculated (m/z)=371.1733. found (m/z)=371.18595.

### tert-Butyl 4-(3-ethoxy-3-oxopropyl)-2-(4-methoxyphenyl)-1H-pyrrole-1-carboxylate (153)

To a round-bottom flask charged with (E)-tert-butyl 4-(3-ethoxy-3-oxoprop-1-enyl)-2-(4-methoxyphenyl)-1H-pyrrole-1-carboxylate 39 (0.2814 g, 0.758 mmol, 1.0 eq) was added absolute ethanol (6.3 mL). 10% palladium on carbon catalyst (0.0242 g, 0.023 mmol, 0.03 eq) was added to the solution and the flask was evacuated and flushed with hydrogen three times. The suspension was then stirred under hydrogen gas at a pressure of one atmosphere for five hours. The catalyst was filtered using a medium fritted-glass filter and rinsed thoroughly with absolute ethanol. The filtrate was evaporated in vacuo to yield 153 as a solid product (0.2625 g, 0.703 mmol, 92.5% yield). TLC Rf=0.44 (20% ethyl acetate, 80% hexanes). 1H NMR (500 MHz, acetone-d6): δ 1.236 (t, 3H, J=10 Hz), 1.367 (s, 9H), 2.545 (t, 2H, J=7.5 Hz), 2.714 (t, 2H, J=7.5 Hz), 4.107 (q, 2H, J=7 Hz), 6.031 (s, 1H), 6.882 (d, 2H, J=9 Hz), 7.118 (s, 1H), 7.224 (d, 2H, J=9 Hz). 13C NMR (500 MHz, acetone-d6): δ 14.657, 22.864, 27.862, 35.418, 55.617, 60.651, 83.722, 113.841, 115.528, 119.914, 125.496, 127.793, 131.122, 135.906, 150.022, 160.001, 173.150. HRMS (ESITOF) MH+ of C21H27NO5+ calculated (m/z)=373.1889. found (m/z)=373.1978.

### 3-(5-(4-methoxyphenyl)-1H-pyrrol-3-yl)-N,N-dimethylpropanamide (154)

To a round-bottom flask containing a suspension of dimethylammonium chloride (0.1146 g, 1.41 mmol, 2 eq) in anhydrous toluene (7 mL) under argon was added dropwise a solution of trimethylaluminum, 2 M in toluene (0.70 mL, 1.41 mmol, 2 eq). The resulting mixture was stirred for one hour at room temperature. In another roundbottom flask a solution of tert-butyl 4-(3-ethoxy-3-oxopropyl)-2-(4-methoxyphenyl)-1H-pyrrole-1-carboxylate (0.2625 g, 0.702 mmol, 1.0 eq) in anhydrous toluene (6 mL), under argon, was prepared and was subsequently added dropwise to the reaction mixture. The reaction mixture was refluxed for 23 hours. The mixture was allowed to cool to room temperature and then 4.5 mL of 2 M aqueous hydrochloric acid were added. The reaction flask was placed in an ice bath and stirred. The resulting reaction mixture and precipitate were transferred to a separatory funnel. 10 mL deionized water were added to the funnel and the resulting mixture was extracted thrice with 20 mL ethyl acetate and twice with 10 mL dichloromethane. The organic fractions were combined and dried over magnesium sulfate. The solvent was removed in vacuo to yield light yellow crystals (0.1332 g, 0.489 mmol, 69.7% yield). TLC Rf=0.41 (20% ethyl acetate, 80% dichloromethane). 1H NMR (500 MHz, methanol-d4): δ 2.633 (t, 2H, J=6.5 Hz), 2.759 (t, 2H, J=8 Hz), 2.928 (s, 3H), 2.997 (s, 3H), 3.785 (s, 3H), 6.227 (s, 1H), 6.567 (s, 1H), 6.878 (d, 2H, J=9 Hz), 7.436 (d, 2H, J=9 Hz). 13C NMR (500 MHz, chloroform-d3): δ 22.956, 35.361, 35.608, 37.443, 55.477, 105.232, 114.418, 115.877, 124.855, 125.268, 126.143, 132.292, 158.277, 173.079. HRMS (ESI-TOF) MH+ of C16H20N2O2+ calculated (m/z)=272.1525. found (m/z)=272.1598.

### 3-(5-(4-methoxyphenyl)-1H-pyrrol-3-yl)-N,N-dimethylpropan-1-amine (155)

To a round-bottom flask containing a suspension of lithium aluminum hydride (0.0102 g, 0.27 mmol, 7.3 eq) in anhydrous tetrahydrofuran (0.5 mL) under argon and maintained at 0° C. was added a solution of 3-(5-(4-methoxyphenyl)-1H-pyrrol-3-yl)-N,Ndimethylpropanamide (0.0100 g, 0.037 mmol, 1.0 eq) in anhydrous tetrahydrofuran, also under argon. The reaction mixture was stirred under argon at room temperature for two hours. The reaction was quenched by the addition of 3 mL of 1 M aqueous sodium carbonate, and the mixture was transferred to a separatory funnel and extracted exhaustively into ethyl acetate. The organic fractions were combined and dried over sodium sulfate. The solvent was removed in vacuo, yielding a reddish-brown solid (0.0096 g, 0.037 mmol, 100% yield). TLC Rf=0.41 (10% methanol, 90% dichloromethane). 1H NMR (500 MHz, chloroform-d3): δ 1.806 (quintet, 2H, J=7.5 Hz), 2.257 (s, 6H), 2.363 (t, 2H, J=7.5 Hz), 2.524 (t, 2H, J=8 Hz), 3.821 (s, 3H), 6.282 (s, 1H), 6.588 (s, 1H), 6.900 (d, 2H, J=9 Hz), 7.380 (d, 2H, J=8.5 Hz), 8.373 (s, 1H). 13C NMR (500 MHz, chloroform-d3): δ 24.989, 28.974, 45.505, 55.254, 59.679, 105.143, 114.213, 115.697, 125.106, 125.280, 126.305, 132.023, 157.962. HRMS (ESI-TOF) MH+ of C16H22N2O+ calculated (m/z)=258.1732. found (m/z)=258.1806.

### 3-(3-(dimethylamino)propyl)-5-(4-methoxyphenyl)-1H-pyrrole-2-carbaldehyde (156)

To a round-bottom flask containing phosphoryl chloride (0.42 mL, 4.62 mmol, 10 eq) under argon was added anhydrous dimethylformamide (0.71 mL, 9.23 mmol, 20 eq). The solution was stirred under argon at room temperature for one hour, after which 1,2-dichloroethane (20 mL) was added. In another round-bottom flask, a solution of 3-(5-(4-methoxyphenyl)-1H-pyrrol-3-yl)-N,N-dimethylpropan-1-amine (0.1192 g, 0.462 mmol, 1.0 eq) in 1,2-dichloroethane (25 mL) was prepared and flushed with argon. To this second flask were added 5 mL of the phosphoryl chloride/dimethylformamide solution and the resulting reaction solution was placed in an oil bath (preheated to 90° C.) and refluxed for four hours. The reaction flask was removed from the oil bath, crushed ice (deionized) was added to the flask, and the reaction mixture was subsequently brought to pH 12 by the addition of 10 M aqueous sodium hydroxide. The reaction mixture was placed in an oil bath (preheated to 70° C.) for one hour and then allowed to cool down to room temperature. The crude mixture was transferred to a separatory funnel and extracted exhaustively into ethyl acetate. The organic fractions were combined and dried over magnesium sulfate. The solvent was removed in vacuo. The resulting solid was purified by column chromatography on Davisil (stepwise: 10% isopropanol, 90% dichloromethane with 0.1% triethylamine added per liter solvent; 20% isopropanol, 80% dichloromethane with 0.1% triethylamine added per liter solvent), yielding a brownish solid (0.0844 g, 0.295 mmol, 63.8% yield). TLC Rf=0.26 (40% isopropanol, 60% dichloromethane). 1H NMR (500 MHz, chloroform-d3): δ 1.854 (quintet, 2H, J=7.5 Hz), 2.247 (s, 6H), 2.349 (t, 2H, J=8 Hz), 2.812 (t, 2H, J=8 Hz), 3.850 (s, 3H), 6.397 (s, 1H), 6.955 (d, 2H, J=9 Hz), 7.559 (d, 2H, J=8.5 Hz), 9.602 (s, 1H). 13C NMR (500 MHz, chloroform-d3): δ 23.111, 29.350, 45.462, 55.394, 59.005, 108.588, 114.481, 123.524, 127.002, 129.672, 140.190, 159.993, 176.709. HRMS (ESI-TOF) MH+ of C17H22N2O2+ calculated (m/z)-286.1681. found (m/z)=286.1754.

### 3-(2-formyl-5-(4-methoxyphenyl)-1H-pyrrol-3-yl)-N,N,N-trimethylpropan-1-aminium iodide (157)

To a round-bottom flask containing 3-(3-(dimethylamino)propyl)-5-(4-methoxyphenyl)-1H-pyrrole-2-carbaldehyde 138 (0.0844 g, 0.295 mmol, 1.0 eq) in anhydrous dichloromethane (0.6 mL) under argon was added iodomethane (0.18 mL, 2.95 mmol, 10 eq). The mixture was stirred at room temperature for one and one-half hours. The excess iodomethane and solvent were removed in vacuo, yielding 157 as an off-white solid (0.09 72 g, 0.227 mmol, 76.9% yield). 1H NMR (500 MHz, methanol-d4): δ 2.197 (quintet, 2H, J=3.5 Hz), 2.934 (t, 2H, J=7.5) 3.170 (s, 9H), 3.465 (t, 2H, J=9.5 Hz), 3.830 (s, 3H), 6.632 (s, 1H), 6.988 (d, 2H, J=9 Hz), 7.697 (d, 2H, J=8.5 Hz), 9.594 (s, 1H). 13C NMR (500 MHz, methanol-d4): δ 28.582, 53.897, 56.018, 67.583, 115.627, 124.912, 128.214, 130.929, 131.022, 141.074, 161.732. HRMS (ESI-TOF) MH+ of C18H25N2O2+ calculated (m/z)=301.1911. found (m/z)=301.1894.

### (Z)-3-(2-((5-(2-carboxyethyl)-3-methyl-2H-pyrrol-2-ylidene)methyl)-5-(4-methoxyphenyl)-1H-pyrrol-3-yl)-N,N,N-trimethylpropan-1-aminium chloride (158)

To a round-bottom flask containing a stirred solution of 3-(2-formyl-5-(4-methoxyphenyl)-1H-pyrrol-3-yl)-N,N,N-trimethylpropan-1-aminium iodide (0.0116 g, 0.027 mmol, 1.0 eq) and 3-(4-methyl-1H-pyrrol-2-yl)propanoic acid 140 (0.0046 g, 0.03 mmol, 1.1 eq) in absolute ethanol (0.9 mL) was added p-toluenesulfonic acid monohydrate (0.0052 g, 0.027 mmol, 1.0 eq), and the reaction solution was stirred under argon at room temperature for one hour. The reaction mixture was then passed through a DOWEX 21K Cl anion exchange resin and eluted with deionized water. The eluate was evaporated in vacuo to yield a 158 as red solid (0.012 g, 0.025 mmol, 92.6%). 1H NMR (500 MHz, methanol-d4): δ 2.268 (quintet, 2H, J=7.5 Hz), 2.454 (s, 3H), 2.819 (t, 2H, J=7 Hz), 2.960 (t, 2H, J=7.5 Hz), 3.127 (t, 2H, J=7.5 Hz), 3.187 (s, 9H), 3.505 (t, 2H, J=8.5 Hz), 3.901 (s, 3H), 6.479 (s, 1H), 7.099 (d, 2H, J=7 Hz), 7.163 (s, 1H), 7.481 (s, 1H), 8.017 (d, 2H, J=7 Hz). 13C NMR (500 MHz, methanol-d4): δ 12.358, 23.966, 24.747, 25.041, 33.153, 53.832, 56.311, 67.239, 116.136, 118.070, 122.150, 122.407, 130.151, 130.396, 130.806, 148.987, 150.867, 155.391, 159.390, 164.378, 175.679. HRMS (ESI-TOF) MH+ of C26H34N3O3+ calculated (m/z)=436.2595. found (m/z)=436.2639.

### 3-(2-carboxyethyl)-5,5-difluoro-7-(4-methoxyphenyl)-1-methyl-9-(3-(trimethylammonio)propyl)-5H-dipyrrolo[1,2-c:1′,2′-f][1,3,2]diazaborinin-4-ium-5-uide 2,2,2-trifluoroacetate (159)

To a round-bottom flask containing a stirred solution of (Z)-3-(2-((5-(2-carboxyethyl)-3-methyl-2H-pyrrol-2-ylidene)methyl)-5-(4-methoxyphenyl)-1H-pyrrol-3-yl)-N,N,N-trimethylpropan-1-aminium chloride 141 (0.010 g, 18 μmol, 1.0 eq) in anhydrous dichloromethane (2 mL) at 0° C., under argon, was added diisopropylethylamine (0.047 g, 364 μmol, 20 eq) and the reaction mixture was stirred for forty-five minutes at 0° C. Boron trifluoride diethyl etherate complex (0.013 g, 91 μmol, 5 eq) was added dropwise, and the mixture was stirred at 0° C. for two hours. The solvent was removed in vacuo at 0° C. The resulting reddish residue was purified by HPLC (20%-60% B in 40 minutes, 500 nm, product eluted in 12% B), yielding 159 as a dark reddish product (0.0048 g, 8.0 μmol, 44.6%) 1H NMR (500 MHz, 1:1 deuterium oxide:acetonitrile-d3): δ 2.578 (quintet, 2H, J=4 Hz), 2.785 (s, 3H), 3.168 (t, 2H, J=7.5 Hz), 3.260 (t, 2H, J=7.5 Hz), 3.516 (s, 9H), 3.575 (t, 2H, J=8.5 Hz), 3.792 (t, 2H, J=7.5 Hz), 4.340 (s, 3H), 6.774 (s, 1H), 7.122 (s, 1H), 7.524 (d, 2H, J=8.5

Hz), 8.027 (s, 1H), 8.356 (d, 2H, J=9 Hz).

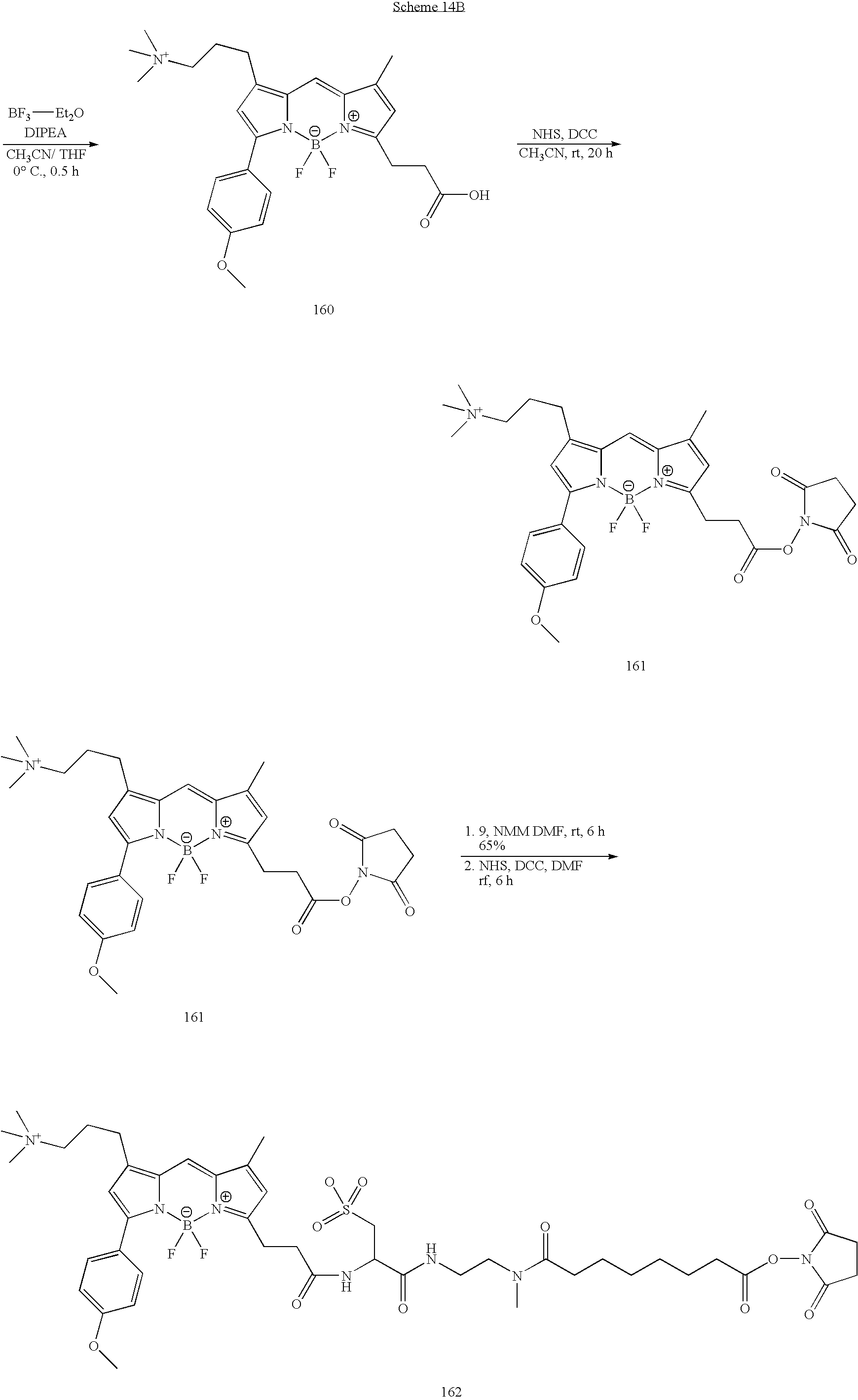

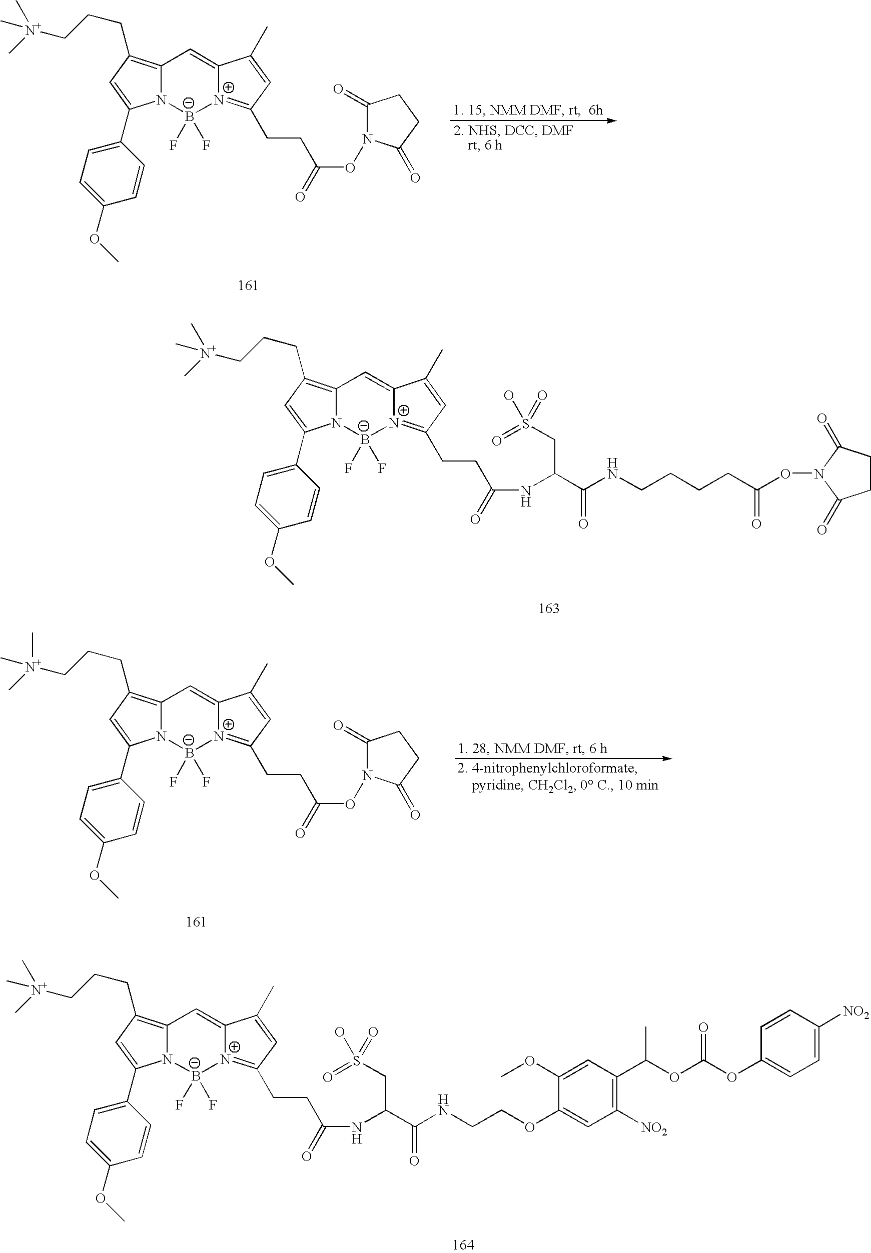

3-(2-carboxyethyl)-5,5-difluoro-7-(4-methoxyphenyl)-1-methyl-9-(3-(trimethylammonio)propyl)-5H-dipyrrolo[1,2-c:1′,2′-f][1,3,2]diazaborinin-4-ium-5-uide

2,2,2-trifluoroacetate, succinimidyl ester (160)

A round-bottom flask was charged with 3-(2-carboxyethyl)-5,5-difluoro-7-(4-methoxyphenyl)-1-methyl-9-(3-(trimethylammonio)propyl)-5H-dipyrrolo[1,2-c:1′,2′-f][1,3,2]diazaborinin-4-ium-5-uide 2,2,2-trifluoroacetate 159 (0.0033 g, 5.5 μmol, 1.0 eq), N-hydroxysuccinimide (0.0064 g, 55.0 μmol, 10 eq), and dicyclohexylcarbodiimide (0.0125 g, 61.0 μmol, 11.0 eq) and flushed with argon. The solid reagents were then dissolved in 0.5 mL anhydrous acetonitrile and the reaction solution was stirred at room temperature for 18 hours.

The solvent was removed in vacuo. The crude residue was purified by HPLC (35%-55% B in 40 minutes, 500 nm, product 160 eluted in 44% B) (0.035 g, 0.0052 mmol, 94.5%). 1H NMR (500 MHz, methanol-d4): δ 2.204 (quintet, 2H, J=8 Hz), 2.350 (s, 3H), 2.842 (s, 3H), 2.871 (t, 2H, J=7.5 Hz), 3.056 (t, 2H, J=7.5 Hz), 3.148 (s, 9H), 3.283 (t, 2H, J=7.5 Hz), 3.425 (t, 3H, J=9 Hz), 3.868 (s, 3H), 6.351 (s, 1H), 6.710 (s, 1H), 7.004 (d, 2H, J=9 Hz), 7.588 (s, 1H), 7.947 (d, 2H, J=8.5 Hz).

### 3-(6-(2-β5-carboxypentyl)(methyl)ammonio)ethylamino)-3,6-dioxo-5-(sulfonatomethyl)hexyl)-5,5-difluoro-7-(4-methoxyphenyl)-1-methyl-9-(3-(trimethylammonio)propyl)-5H-dipyrrolo[1,2-c:1′,2′-f][1,3,2]diazaborinin-4-ium-5-uide 2,2,2-trifluoroacetate (161)

3-(2-carboxyethyl)-5,5-difluoro-7-(4-methoxyphenyl)-1-methyl-9-(3-(trimethylammonio)propyl)-5H-dipyrrolo[1,2-c:1′,2′-f][1,3,2]diazaborinin-4-ium-5-uide 2,2,2-trifluoroacetate, succinimidyl ester 160 (3.5 mg, 5.0 μmol, 1.0 eq) and (2R)-2-amino-3-(2-((5-carboxypentyl)(methyl)ammonio)ethylamino)-3-oxopropane-1-sulfonate (11 mg, 25 μmol, 5 eq) were added to a 10 mL conical flask equipped with a magnetic stir vane. The flask was flushed with argon. Anhydrous dimethylformamide (0.5 mL) and freshly distilled N-methylmorpholine (11 μL, 100 μmol, 20 eq) were added to the flask and the solution was stirred at room temperature for 3 hrs. The solvent was removed in vacuo. The crude mixture was purified by HPLC to yield 161 as a deeply colored red solid (3.1 mg, 3.4 μmol, 67.5%). 1H NMR (500 MHz, methanol-d4): δ 1.35-1.45 (m, 2H), 1.60-1.70 (m 2H), 1.70-1.80 (m, 2H), 2.19 (m, 2H), 2.28-2.37 (m, 5H), 2.60-2.75 (m, 2H), 2.83-2.89 (m, 5H), 2.95-3.10 (m, 1H), 3.12-3.21 (m, 12H), 3.35 (m, 5H) 3.40-3.46 (m, 2H), 3.52 (s, br, 1H), 3.67 (s, br, 1H), 3.89 (s, 3H), 4.60-4.66 (m, 1H), 6.28 (s, 1H), 6.68 (s, 1H), 7.00 (d, 2H, J=9 Hz), 7.57 (s, 1H), 7.92 (d, 2H, J=9 Hz).

### 3-(6-(2-β5-carboxypentyl)(methyl)ammonio)ethylamino)-3,6-dioxo-5-(sulfonatomethyl)hexyl)-5,5-difluoro-7-(4-methoxyphenyl)-1-methyl-9-(3-(trimethylammonio)propyl)-5H-dipyrrolo[1,2-c:1′,2′-f][1,3,2]diazaborinin-4-ium-5-uide

2,2,2-trifluoroacetate, succinimidyl ester (162) 3-(6-(2-((5-carboxypentyl)(methyl)ammonio)ethylamino)-3,6-dioxo-5-(sulfonatomethyl)hexyl)-5,5-difluoro-7-(4-methoxyphenyl)-1-methyl-9-(3-(trimethylammonio)propyl)-5H-dipyrrolo[1,3,2]diazaborinin-4-ium-5-uide-2,2,2-trifluoroacetate 161 (1.2 mg, 1.3 μmol, 1.0 eq), N-hydroxysuccinimide (3.0 mg, 26 μmol, 20 eq), and dicyclohexylcarbodiimide (5.4 mg, 26 μmol, 20 eq) were added to a 5 mL conical flask equipped with a magnetic stir vane. The flask was flushed with argon. Anhydrous dimethylformamide (0.2 mL) was added to the flask and the solution was stirred overnight at room temperature. The reaction mixture was filtered and the solvent removed in vacuo. The crude mixture was purified by HPLC to yield 162 as a deeply colored red solid (1.1 mg, 1.1 μmol, 84.7%). 1H NMR (500 MHz, acetonitrile-d3): δ 1.40-1.50 (m, 2H), 1.68-1.78 (m, 4H), 2.05-2.15 (m, 4H), 2.60-2.68 (m, 6H), 2.71-2.81 (m, 10H), 2.85-2.99 (m, 3H), 3.01 (s, 9H), 3.05-3.15 (m, 4H), 3.25-3.37 (m, 4H), 3.89 (s, 3H), 4.52 (s, br, 1H), 6.30 (s, 1H), 6.63 (s, 1H), 7.04 (d, 2H, J=9 Hz), 7.50 (s, 1H), 7.90 (d, 2H, J=9 Hz). HRMS (ESI-TOF) MH+ of C42H59BF2N7O10S+ calculated (m/z)=902.4103. found (m/z)=902.4099.

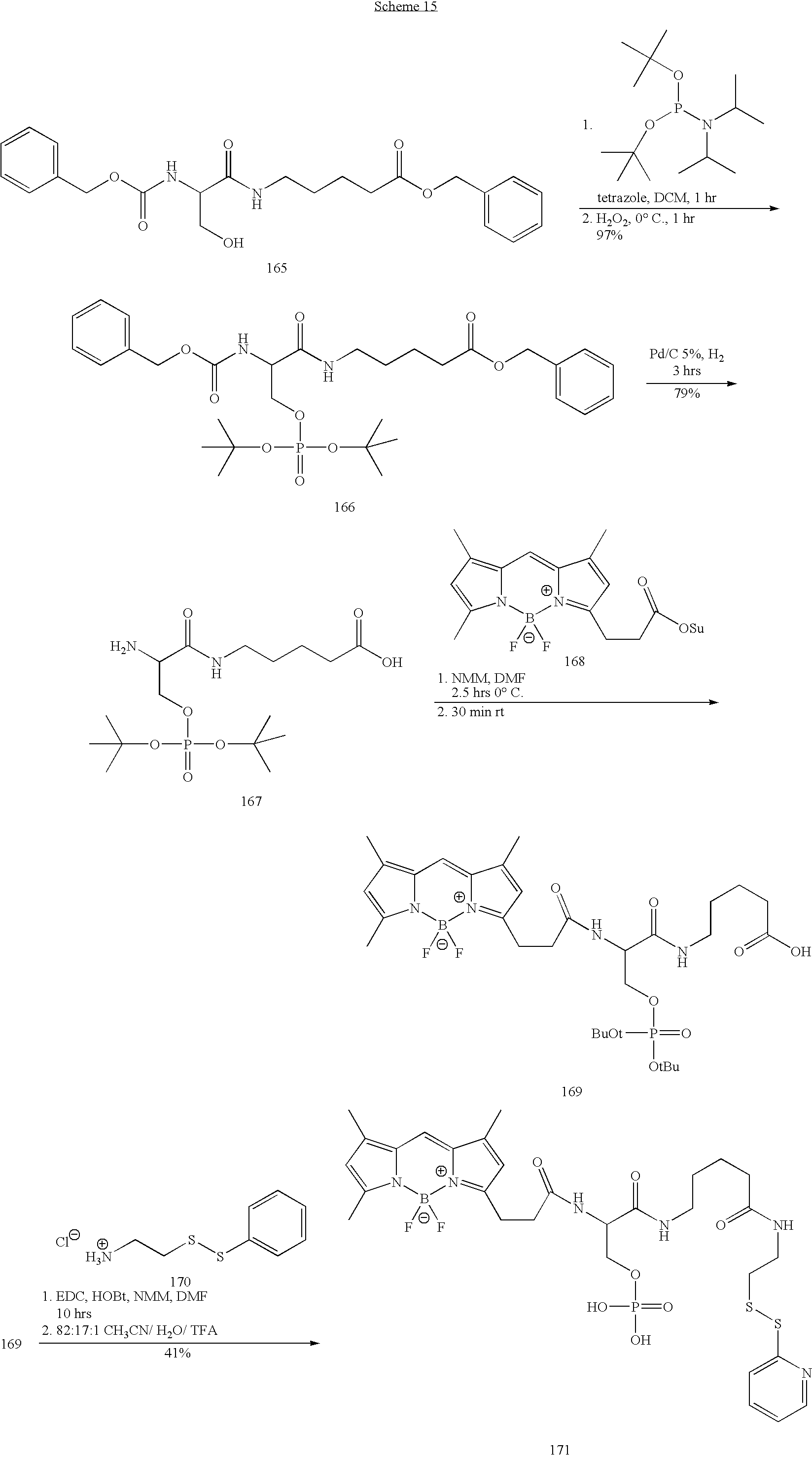

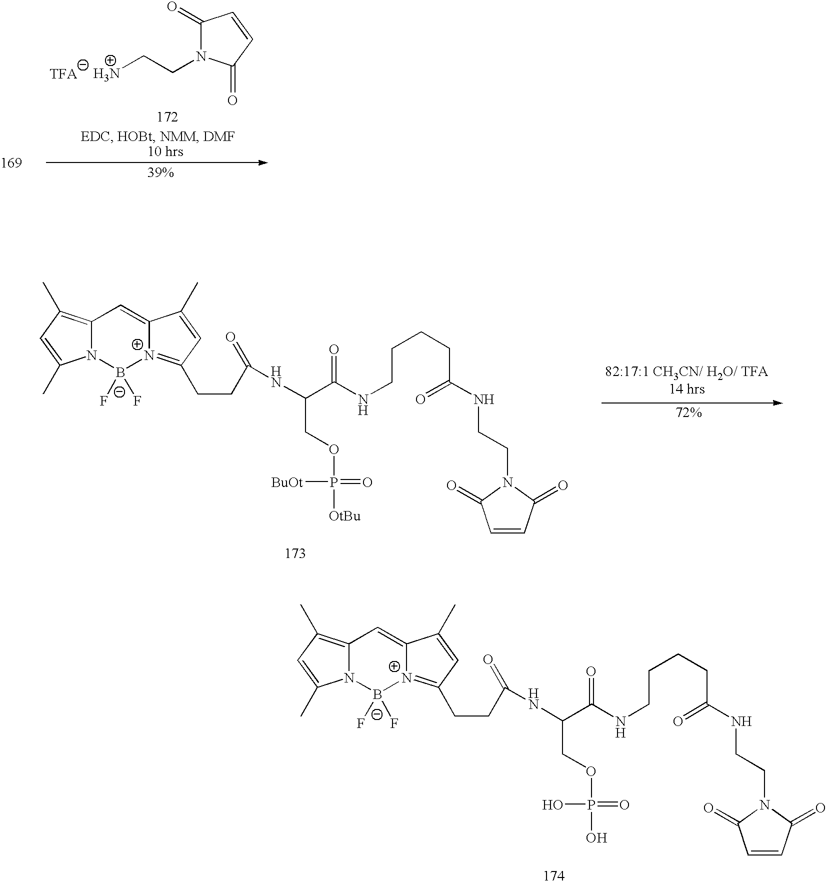

(166): To an argon flushed flask containing 165 (200 mg, 0.47 mmol) and dichloromethane to was added di-tert-butyl-diisopropylphosphoramidite (0.24 mL, 0.74 mmol, 1.6 eq) followed by tetrazole (106 mg, 1.52 mmol, 3.3 eq). The solution was stirred under argon for 1 hour at room temperature and then cooled to 0° C. Then, hydrogen peroxide (0.06 mL, 50%) was added and the solution was stirred for an additional hour at 0° C. Dichloromethane (20 mL) was added and the solution was washed sequentially with 10% sodium metabisulfite (2×5 mL), saturated sodium bicarbonate (2×5 mL), water (1×5 mL), and brine (1×5 mL). The combined organic solution was dried over Na₂SO₄ and then removed by rotary evaporation. The remaining clear oil was frozen and lyophilized to yield 277 mg (97% yield). ¹H NMR (300 MHz, CD₃CN) δ 7.36 (m, 10H), 6.99 (br s, 1H), 6.56 (br s, 1H), 5.09 (m, 4H) 4.28-4.24 (m, 1H) 4.20-4.13 (m, 2H), 3.18-3.12 (q, 2H, J=6.5 Hz), 2.37-2.32 (t, 2H, J=7 Hz), 1.61-1.51 (m, 2H), 1.50-1.42 (m, 20H). ¹³C NMR (125 MHz, CD₃CN) δ 22.92, 29.61, 30.08, 30.13, 30.60, 34.33, 39.71, 56.48, 56.57, 66.72, 67.51, 83.94, 83.99, 128.93, 129.02.

(167): To an argon flushed flask containing 5% Palladium on carbon (85 mg, 0.04 mmol, 0.3 eq), was added a solution of 166 (82 mg, 0.12 mmol) in methanol (3 mL). The solution was sparged with a balloon of hydrogen gas three times and then let stir under H₂ for 3 hours. The solution was then filtered and the solvent was removed by rotary evaporation. The resulting clear oil was frozen and lyophilized for 12 hours affording 40 mg of clear wax (79%). ¹H NMR (500 MHz, CD₃OD) δ 4.15-4.10 (m, 2H), 3.69-3.68 (t, 1H, J=4.4 Hz), 3.30-3.18 (m, 2H), 2.28-2.25 (t, 2H, J=7.1 Hz), 1.66-1.61 (m, 2H), 1.59-1.55 (m, 2H), 1.49 (s, 18H). ¹³C NMR (CD₃OD, 125 MHz) δ 24.90, 29.99, 30.26, 35.45, 40.94, 68.54, 85.35, 170.68, 178.37. ³¹P NMR (121 MHz, CD₃OD) δ −11.77 vs 85% H₃PO₄.

(169): To an argon flushed flask containing 168 (48 mg, 0.119 mmol) in an ice bath was added a solution of 167 (81 mg, 0.20 mmol, 1.7 eq) in anhydrous DMF (3 mL). Then freshly distilled NMM (26.7 uL, 0.25 mmol, 2.1 eq) was added and the solution was stirred for 2.5 hours at which time the ice bath was removed and stirred for another 30 minutes. The reaction was followed by TLC(Rf=0.5, 14:1 DCM/MeOH). The solution was then frozen and lyophilized. The resulting orange red solid was purified by column chromatography (premium Rf silica gel, 100:1 to 10:1 DCM/MeOH). The solvent was removed by rotary evaporation at 0° C. to afford 60 mg of orange-red solid (73% yield). ¹H NMR (300 MHz, CD₃OD) δ 7.40 (s, 1H), 6.17 (s, 1H), 6.13 (s, 1H), 4.60-4.59 (m, 1H), 4.14-4.13 (m, 2H), 3.16 (m, 4H), 2.70-2.66 (t, 2H, J=7.5), 2.45 (s, 3H), 2.27 (m, 8H), 1.54-1.49 (m, 4H), 1.45 (s, 18H). ¹³C NMR (125 Hz, CD₃OD) δ 11.31, 11.41, 14.84, 23.37, 25.39, 29.89, 34.54, 35.74, 40.35, 55.07, 67.12, 85.05, 118.64, 120.54, 122.80, 134.61, 135.10, 143.07, 144.27, 158.90, 159.50, 170.87, 171.2, 174.75. ³¹P NMR (121 MHz, CD₃OD) 8-11.61 vs 85% H₃PO₄ HRMS-EI (m/z): [M] calcd for C₃₁H₄₈BF₂N₄O₈P+H⁺ 685.3349. found 685.3332.

(171): To an argon flushed flask submerged in an ice bath containing 170 (10.2 mg, 14.9 umol), 169 (3.3 mg, 14.9 umol, 1 eq), and HOBt (2 mg, 14.9 umol) was added anhydrous DMF (0.5 mL) followed by freshly distilled NMM (3.28 uL, 29.8 umol, 2 eq). EDC (2.86 mg, 14.9 umol, 1 eq) was then added and after 2 hours, the ice bath was removed and the solution was stirred under argon for an additional 9 hours. The solvent was then removed in vacuo and the resulting orange-red solid was cooled to 0° C. under argon. Then 1 mL of a 71:28:1 H₂O/CH₃CN/TFA was added. After 5 minutes, the ice bath was removed and the solution was stirred for an additional 22 hours followed by purification by reversed phase HPLC (B 30% to 60% over 30 min, flow=20 mL/min, 450 nm, tᵣₑₜₑₙₜᵢₒₙ=14 min) to afford 4.5 mg (41%). ¹H NMR (500 MHz, CD₃OD) δ 8.35 (s, 1H), 7.78-7.75 (m, 2H), 6.16 (s, 1H), 6.11 (s, 1H), 4.50 (m, 1H), 4.13-4.09 (m, 2H), 3.43-3.41 (m, 2H), 3.15-3.28 (m, 2H), 2.89-2.86 (t, 2H, J=6.6 Hz), 2.68 (m, 2H), 2.43 (s, 3H), 2.25 (m, 8H), 2.15-2.14 (t, 2H, J=7.4 Hz), 1.57 (m, 2H), 1.47 (m, 2H). ³¹P NMR (121 MHz, CD₃OD) δ −0.89 vs H₃PO₄. FIRMS-EI (m/z): [M] calcd for C₃₀H₄₀BF₂N₆O₇PS₂+H⁺ 741.2273. found 741.2296.

(173): To an argon flushed flask submerged in an ice bath containing 172 (27 mg, 39.4 umol), 169 (10 mg, 39.4 umol, 1 eq), and HOBt (5.32 mg, 39.4 umol, 1 eq) was added anhydrous DMF (1 mL) followed by freshly distilled NMM (8.66 uL, 78.8 umol, 2 eq). EDC (7.5 mg, 39.4 umol, 1 eq) was then added and after 2 hours, the ice bath was removed and the solution was stirred under argon for an additional 8 hours. The solvent was then removed in vacuo and the resulting orange-red solid purified by reversed phase HPLC (B 30% to 70% 14 min) to afford 12.3 mg (39% yield). ¹H NMR (500 MHz, CD₃OD) 7.44 (s, 1H), 6.80 (s, 2H), 6.19 (s, 1H), 6.16 (s, 1H), 4.64 (m, 1H), 4.16 (m, 2H), 3.59 (t, 2H, J=5.3 Hz), 3.34 (m, 2H), 3.18 (m, 4H), 2.72 (m, 2H), 2.47 (s, 3H), 2.29 (m, 6H), 2.10 (t, 2H, J=7.1 Hz) 1.60-1.52 (m, 4H), 1.48 (m, 20H). ¹³C NMR (125 Hz, CD₃OD) δ 11.31, 11.45, 14.87, 24.03, 25.43, 29.80, 30.27, 35.72, 36.54, 38.57, 38.92, 40.30, 54.91, 67.52, 85.06, 118.63, 120.60, 122.83, 127.30, 134.61, 135.34, 143.06, 144.27, 158.95, 159.50, 170.88, 172.73, 174.76, 176.28 ³¹P NMR (121 MHz, CD₃OD) δ −11.63 vs 85% H₃PO₄ HRMS-EI (m/z): [M] calcd for C₃₇H₅₄BF₂N₆O₉P+H⁺ 807.3830. found 807.3849.

(174): To an argon flushed flask containing 173 (7.1 mg, 8.8 umol) was added 0.6 mL of a 82:17:1 H₂O/CH₃CN/TFA solution. The solution was stirred for 14 hours and then frozen and lyophilized. The resulting orange-red solid was purification by reversed phase HPLC (B 30% to 70% over 30 min, flow=20 mL/min, PDA detector, tᵣₑₜₑₙₜᵢₒₙ=11.7 min) to afford 4.4 mg (72% yield). ¹H NMR (500 MHz, CD₃OD) δ 7.38 (s, 1H), 6.75 (s, 2H), 6.15 (s, 1H), 6.10 (s, 1H), 4.55 (m, 1H), 4.12 (m, 2H), 3.54 (t, 2H, J=5.2 Hz), 3.30 (m, 2H), 3.14 (m, 4H), 2.66 (m, 2H), 2.41 (s, 3H), 2.24 (m, 6H), 2.06 (t, 2H, J=7.1 Hz), 1.51-1.42 (m, 4H). HRMS-EI (m/z): [M] calcd for C₂₉H₃₆BF₂N₆O₉P⁻² 346.1177. found 346.1170.

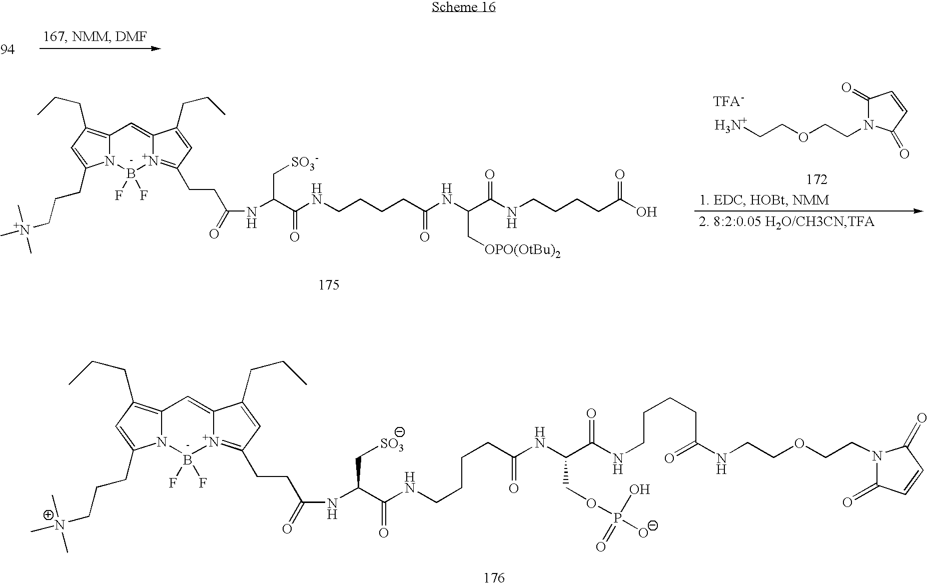

(175): To an Argon flushed flask containing 94 (20 mg, 25.2 umoles), 167 (30 mg, 75.7 umoles, 3 eq), and anhydrous DMF (1 mL) was added NMM (11 uL, 100.8 umoles, 4 eq) and stirred for 22 hours at which time the reaction was complete by TLC (4:1 DCM/MeOH, Rf=0.5, compound 1 Rf=0.6). The solvent was removed by vacuum and the resulting orange solid was purified by reversed phase HPLC (B 30% to 60% over 30 min, flow=20 mL/min, λ=450 nm, retention=18 min) to afford 20.2 mgs (75%). ¹H NMR (300 MHz, CDCl₃) δ 7.46 (s, 1H), 6.30 (s, 2H), 4.60 (m5, 2H), 4.14 (m, 2H), 3.40 (m, 2H), 3.15 (m, 17H), 2.95 (t, 2H, J=7.4 Hz), 2.65 (m, 6H), 1.70-1.40 (m, 30H), 0.97 (m, 6H). HRMS-EI (m/z): [M] calcd for C₄₈H₈₁BF₂N₇O₁₃PSH+ 1076.5485. found 1076.5481.

(176): To an argon flushed flask containing 175 (15.2 mg, 14 umol), 172 (17 mg, 56 umol, 4 eq), and HOBt (7.6 mg, 56 umol, 4 eq), was added sequentially DMF (0.5 mL), NMM (12.4 uL, 113 umol, 8 eq), and EDC (11 mg, 56 umol, 4 eq) at 0° C. The solution was stirred for 45 minutes at which time the ice bath was removed. The solution was allowed to warm to room temperature and continued stirring for another 23 hours at which time the solvent was removed by vacuum. The resulting orange solid was purified by reversed phase HPLC (B 30% to 60% over 30 min, flow=20 mL/min, λ=450 nm, retention=12 min) to afford 5.9 mgs (37%). ¹H NMR (300 MHz, CDCl₃) δ 7.46 (s, 1H), 6.80 (s, 2H), 6.31 (s, 1H), 6.30 (s, 1H), 4.64 (t, 1H, J=6 Hz), 4.55 (t, 1H, J=4.7 Hz), 4.17 (m, 2H), 3.69-3.39 (m, 10H), 3.29 (m, 8H), 3.19 (m, 9H), 2.97 (m, 2H), 2.66 (m, 6H), 2.28-2.15 (m, 6H), 1.70-1.40 (m, 12H), 0.98 (m, 6H). HRMS-EI (m/z): [M] calcd for C₄₈H₇₅BF₂N₉O₁₅PSNa+ 1152.4796. found 1152.4742.

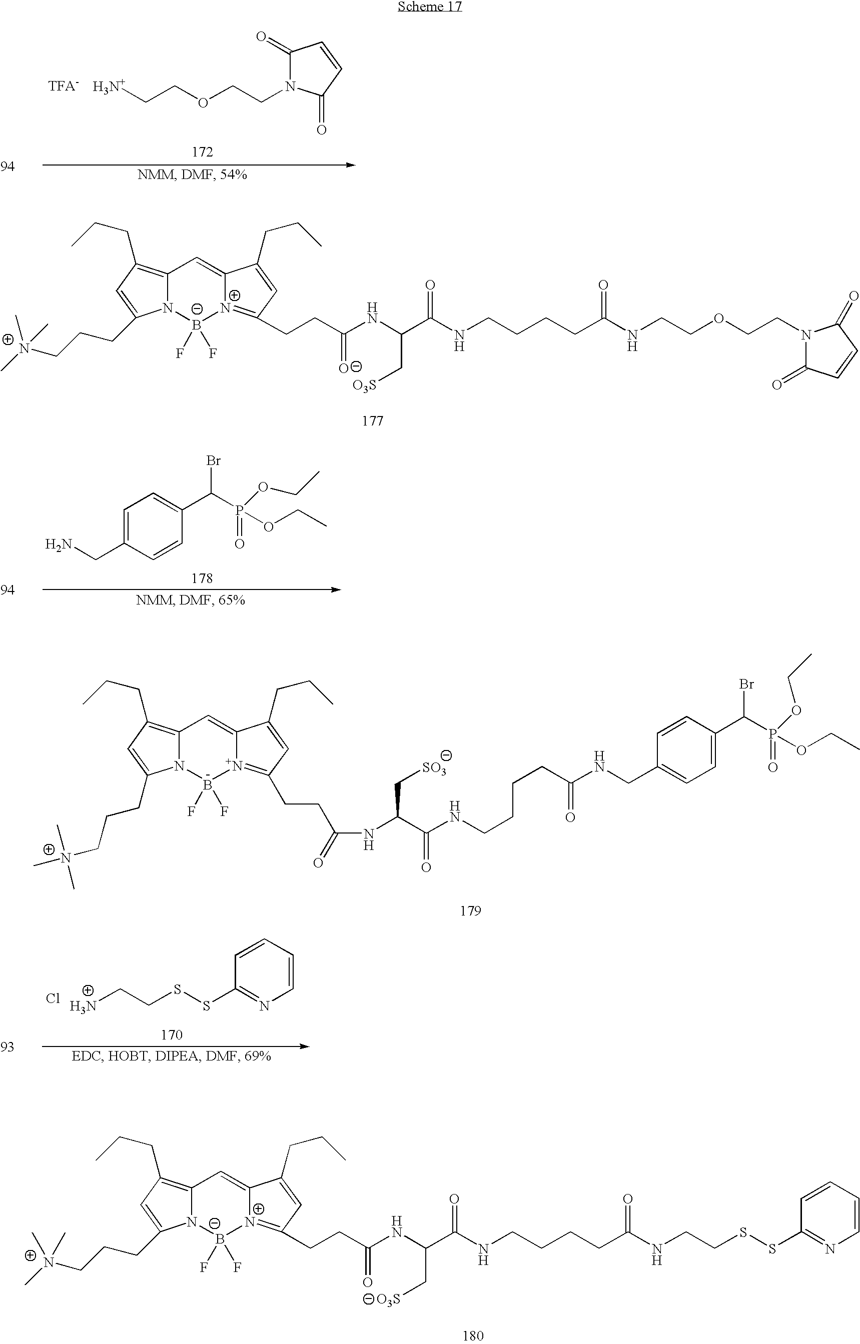

177: To a stirred solution of NHS ester 94 (21 mg, 0.026 mmol) and maleimide 172 (22 mg, 0.072 mmol) in DMF (2.6 mL) at room temperature was added 4-methylmorpholine (0.06 mL, 0.55 mmol). After 24 h, reaction was put on lyopilizer to remove DMF and the crude mixture was purified by HPLC, provided ZBB-mal 177 (12.3 mg, 0.014 mmol) as a fine solid.

(30% B to 60% B over 30 min, 20 mL/min flow, λ=450 nm, product Rt=14.2 min).

¹H NMR (CDCl₃, 500 MHz): δ 7.46 (s, 1H), 6.80 (s, 2H), 6.29 (brs, 2H), 4.63 (dd, J=8.5 Hz, J=5.5 Hz, 1H), 3.63 (t, J=5.0 Hz, 2H), 3.55 (t, J=5.0 Hz, 2H), 3.47 (m, 2H), 3.40 (m, 2H), 3.27 (m, 2H), 3.17 (m, 6H), 3.14 (s, 9H), 2.96 (t, J=8.0 Hz, 2H), 2.66 (m, 6H), 2.21 (m, 4H), 1.64 (m, 6H), 1.49 (m, 2H), 0.98 (m, 6H).

179: To a stirred solution of NHS ester 94 (18 mg, 0.023 mmol) and PBB 178 (30.5 mg, 0.068 mmol) in DMF (2.2 mL) at room temperature was added 4-methylmorpholine (0.052 mL; 0.47 mmol). After 24 h, reaction was put on lyopilizer to remove DMF and the crude mixture was purified by HPLC, provided ZBB-PBB 179 (15 mg, 0.015 mmol) as a fine solid. (30% B to 60% B over 30 min, 20 mL/min flow, λ=450 nm, product Rt=19.2 min).

¹H NMR (CDCl₃, 500 MHz): δ 7.51 (d, J=8.5 Hz, 2H), 7.47 (s, 1H), 7.26 (d, J=8.5 Hz, 2H), 6.30 (s, 1H), 6.29 (s, 1H), 5.24 (d, J=14.0 Hz, 1H), 4.63 (m, 1H), 4.34 (s, 2H), 4.17 (m, 2H), 3.98 (m, 1H), 3.86 (m, 1H), 3.38 (m, 2H), 3.18 (m, 6H), 3.11 (s, 9H), 2.95 (t, J=7.5 Hz, 2H), 2.66 (m, 6H), 2.22 (m, 4H), 1.64 (m, 6H), 1.50 (m, 2H), 1.30 (t, J=7.0 Hz, 3H), 1.12 ((t, J=7.0 Hz, 3H), 0.97 (m, 6H).

180: To a stirred solution of acid 93 (8.6 mg, 0.0106 mmol), EDC (4.6 mg, 0.0240 mmol), HOBT (0.07 mg, 0.00052 mmol) and amine 170 (4.0 mg, 0.0180 mmol) in DMF (2.0 mL) at room temperature was added DIPEA (0.025 mL, 0.144 mmol). After 24 h, reaction was put on lyopilizer to remove DMF and the crude mixture was purified by HPLC (Synergi-RP-Polar, 250×10 mm, 4 micron; 30% B to 60% B over 30 min, 20 mL/min flow, λ=450 nm, product Rt=15 min) to provide 180 (7.2 mg, 69%) as a powder.

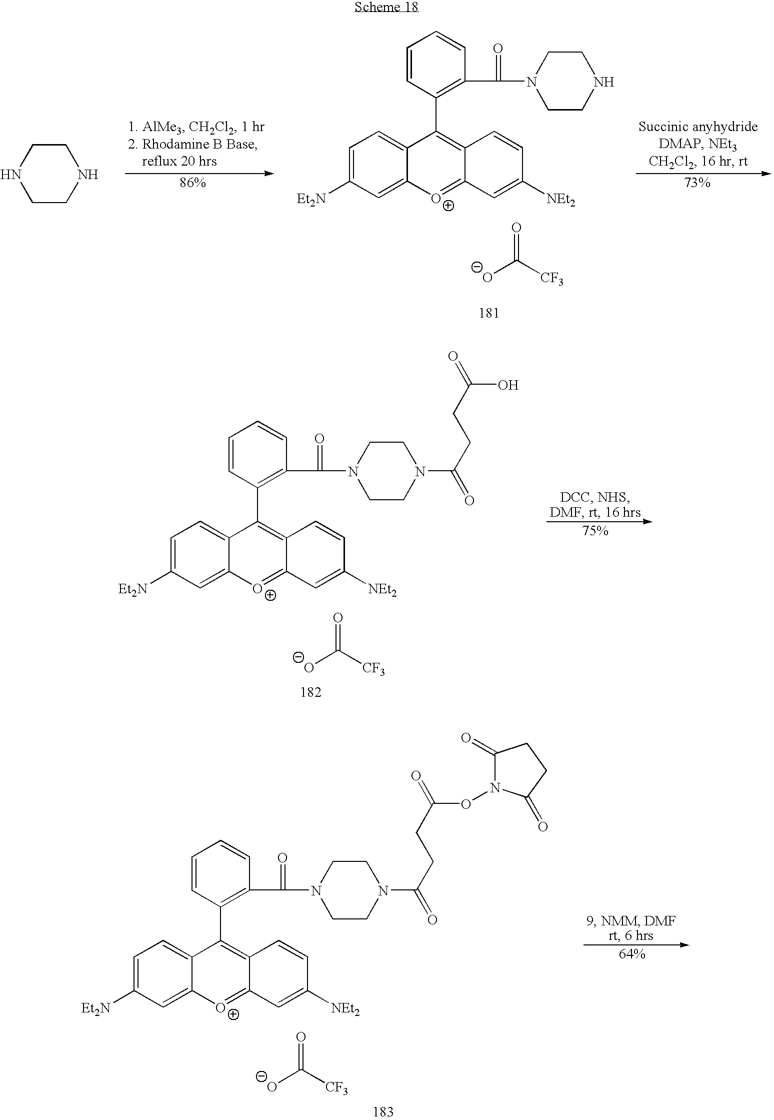

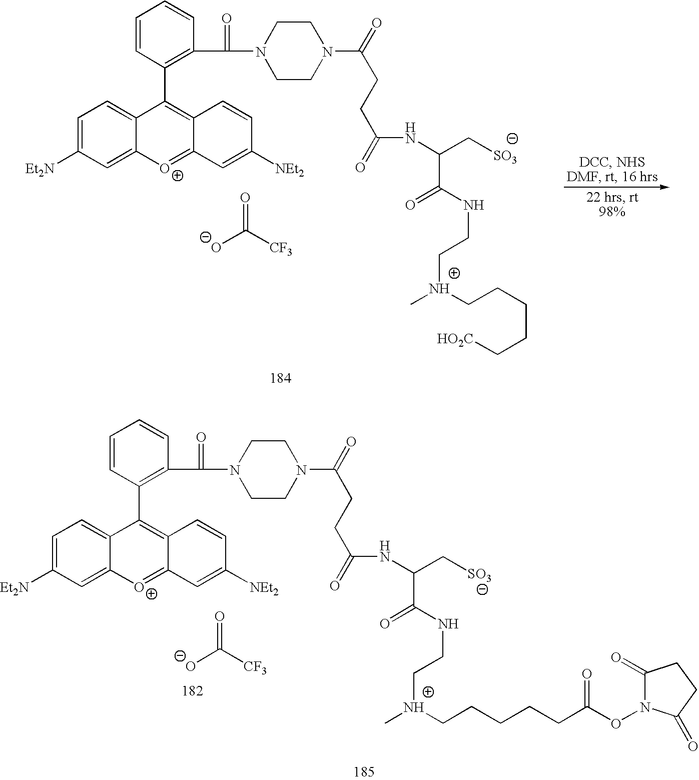

(181): Trimethylaluminum (2.0M in PhMe, 0.23 mL, 0.460 mmol) was added to a solution of piperazine (78 mg, 0.906 mmol) in anhydrous DCM (1 mL) under argon at room temperature. After stirring at room temperature for 1 h, a solution of Rhodamine B base (100 mg, 0.226 mmol) in anhydrous DCM (1 mL) was added dropwise to the solution, and the resulting mixture was refluxed 21 h. The mixture was allowed to cool to room temperature, followed by dropwise addition of 0.1 M HCl (0.2 mL). A precipitate formed, and the precipitate was collected and washed with DCM (1×10 mL) and a 4:1 solution of DCM:MeOH (3×10 mL). The solvents were removed in vacuo and the crude was purified by reverse phase HPLC (30% B to 50% B, 30 min, flow=20 mL/min, λ=525 nm, t_{product}=12 min). The fractions containing the product were frozen and the solvents removed under reduced pressure to provide a red solid (122 mg, 0.195 mmol, 86% yield). mp=116-117° C.; ¹H NMR (500 MHz, CD₃OD) δ 1.28-1.31 (t, 12H, J=7.0 Hz), 3.00-3.10 (br s, 4H), 3.66-3.71 (m, 12H), 6.96 (s, 2H), 7.04-7.06 (d, 2H, J=8.8 Hz), 7.23-7.25 (d, 2H, J=9.5 Hz), 7.50-7.51 (m, 1H), 7.73-7.79 (m, 3H); ¹³C NMR (125 MHz, CD₃OD) 13.4, 45.1, 47.8, 98.2, 115.7, 116.2, 129.1, 131.7, 131.9, 132.3, 133.3, 133.6, 136.5, 157.5, 158.1, 160.1, 170.3.

(182): Distilled triethylamine (35 μL, 0.251 mmol) was added to a solution of 181 (122 mg, 0.195 mmol), DMAP (31 mg, 0.254 mmol) and succinic anhydride (25 mg, 0.250 mmol) in anhydrous DCM (2 mL) under argon and room temperature. The mixture was stirred at room temperature for 17 h, followed by dilution of the mixture in DCM. The organic layer was washed with 1M HCl and brine, and dried over Na₂SO₄. The solvent was removed in vacuo and the crude product purified by reverse phase HPLC (30% B to 60% B, 30 min, flow=20 mL/min, λ=525 nm, t_{product}=18.8 min). The fractions containing the product were frozen and the solvents removed under reduced pressure to afford a red solid (104 mg, 0.143 mmol, 73% yield). mp=94-95° C.; ¹H NMR (300 MHz, CD₃OD) δ 1.29-1.34 (t, 12H, J=6.5 Hz), 2.40-2.42 (t, 2H, J=6.7 Hz), 2.52-2.54 (t, 2H, J=6.7 Hz), 3.67-3.71 (q, 8H, J=7.0 Hz), 6.97 (s, 2H), 7.06-7.1 (d, 2H, J=9.6 Hz), 7.27-7.31 (d, 2H, J=9.2 Hz), 7.52 (s, 1H), 7.72-7.78 (m, 3H); ¹³C NMR (125 MHz, CD₃OD) δ 12.9, 28.8, 30.0, 43.0, 47.0, 97.5, 115.0, 115.2, 128.8, 131.2, 131.7, 132.4, 133.4, 136.7, 157.2, 157.4, 159.4, 169.8, 172.8, 176.5.

(183): A mixture of 182 (30.2 mg, 0.0417 mmol), NHS (38 mg, 0.330 mmol) and DCC (58 mg, 0.281 mmol) in anhydrous DCM (2 mL) was stirred under argon at room temperature for 2.5 h. The solvent was removed in vacuo, and the crude product purified by reverse phase HPLC (40% B to 70% B, 30 min, flow=20 mL/min, λ=525 nm, t_{product}=19 min). The fractions containing the product were frozen and the solvents removed under reduced pressure to afford a red solid (25.8 mg, 0.0314 mmol, 75% yield), ¹H NMR (300 MHz, CD₃CN) δ 1.23-1.27 (t, 12H, J=6.7), 2.65 (br s, 2H), 2.74 (s, 4H), 2.83 (br s, 2H), 3.20-3.35 (m, 8H), 3.60-3.70 (q, 8H, J=6.7), 6.81 (s, 2H), 6.94-6.97 (d, 2H, J=9.5), 7.17-7.19 (d, 2H, J=9.4), 7.42 (s, 1H), 7.62 (s, 1H), 7.71 (s, 2H); ¹³C NMR (125 MHz, CD₃OD) δ 12.9, 26.5, 27.1, 28.3, 46.8, 97.1, 114.7, 128.8, 130.7, 131.0, 131.5, 132.0, 133.1, 136.7, 156.8, 157.1, 158.9, 168.2, 170.0, 171.1.

(184): NMM (68 μL, 0.633 mmol) was added to a solution of 183 (25.8 mg, 0.0314 mmol) and 9 (41 mg, 0.102 mmol) in anhydrous DMF (1.5 mL) under argon and room temperature. The mixture was stirred at room temperature for 3 h before the solvent was removed under reduced pressure. The crude product purified by reverse phase HPLC (40% B to 55% B, 30 min, flow=20 mL/min, λ=525 nm, t_{product}=8 min). The fractions containing the product were frozen and the solvents removed under reduced pressure to afford a red solid (21.0 mg, 0.0201 mmol, 64% yield). ¹H NMR (500 MHz, D₂O) δ 1.16-1.19 (t, 12H, J=7.0 Hz), 1.34 (br s, 2H), 1.58-1.70 (m, 4H), 2.33 (t, 2H, J=4.5 Hz), 2.50-2.61 (m, 4H), 2.85 (s, 3H), 3.05 (m, 2H), 3.2-3.3 (m, 10H), 3.52-3.55 (t, 8H, J=6.6), 3.60 (m, 2H), 4.63 (m, 1H), 6.70 (d, 2H, 5.3 Hz), 6.87 (s, 2H), 7.13 (s, 2H), 7.57-7.63 (m, 2H), 7.78-7.84 (m, 2H).

(185): A solution of 184 (14.0 mg, 0.0134 mmol), NHS (12 mg, 0.104 mmol) and DCC (16 to mg, 0.0775 mmol) in anhydrous DCM (1.3 mL) under argon was stirred at room temperature for 7 h. The solvent was removed in vacuo, and the crude product purified by reverse phase HPLC (40% B to 60% B, 30 min, flow=20 mL/min, λ=525 nm, t_{product}=12 min). The fractions containing the product were frozen and the solvents removed under reduced pressure to afford a red solid (15.0 mg, 0.0131 mmol, 98% yield). ¹H NMR (300 MHz, CD₃OD) δ 1.27-1.32 (t, 12H, J=6.7 Hz), 1.49-1.51 (br s, 2H), 1.78-1.81 (m, 4H), 2.49-2.51 (br s, 2H), 2.66-2.68 (m, 4H), 2.81 (s, 4H), 2.88 (s, 3H), 3.06 (m, 2H), 3.19 (m, 2H), 3.40-3.55 (m, 8H), 3.67-3.69 (q, 8H, J=7.0 Hz), 4.61 (s, 1H), 6.96 (s, 2H), 7.04-7.08 (d, 2H, J=9.0 Hz), 7.25-7.28 (d, 2H, J=9.5 Hz), 7.51 (s, 1H), 7.71-7.76 (m, 3H), 8.38 (s, 1H); HRMS-EI (m/z): [M] calcd for C₅₂H₆₉N₈O₁₂S⁺ 1029.4750. found 1029.4745.

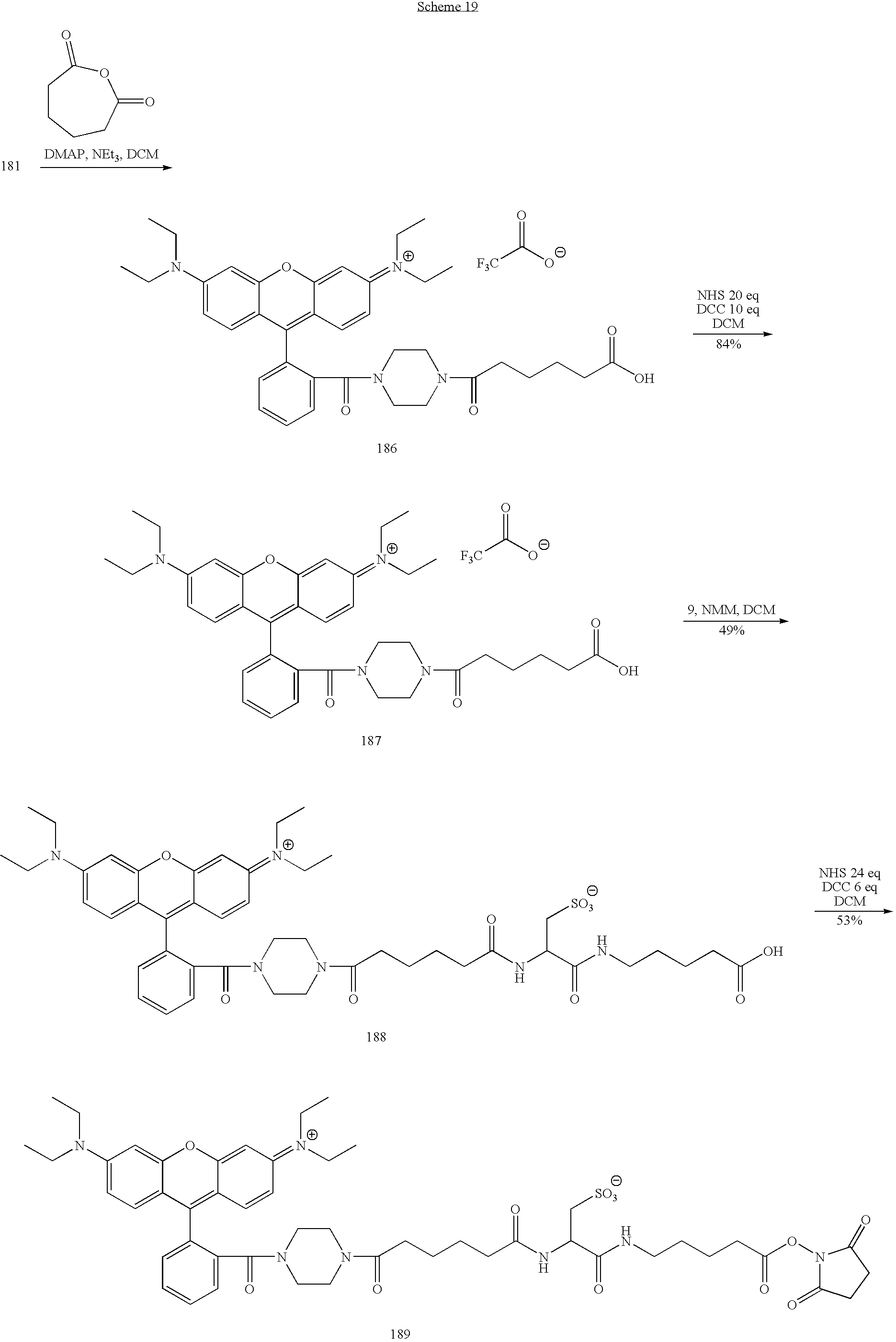

(186): To an argon flushed flask was added 181 (7.42 g, 13.6 mmol) and anhydrous DCM (50 mL). Then adipoyl anhydride (1.93 g, 14.9 mmol, 1.1 eq) and DMAP 1.78 g, 13.6 mmol, 1 eq) were added followed the dropwise addition of NEt₃ (1.9 mL, 13.6 mmol, 1 eq). The reaction was monitored by TLC (2:1 DCM/MeOH R_{f(123)}=0.1, R_{f(128)}=0.9) and after 6 hours, 1 M K₂CO₃ (500 mL) was added and the resulting bilayer was washed with EtOAc (150 mL×3). Then NaCl₍ₛ₎ was added to the aqueous phase followed by extraction with 2:1 iPrOH/DCM until the solution exhibited a light pink color. The combined organic extracts were dried (Na₂SO₄) and the solvent was removed by vacuum. The remaining red solid was dissolved in CHCl₃ and filtered. After removal of the solvent by vacuum, the resulting crude solid was purified by RP HPLC (multiple injections, B 40% to 70% over 30 min, flow=20 mL/min, λ=530 nm, retention=19.6 min) to afford the pure product as a red solid (3.50 g, 38%). ¹H NMR (300 MHz, CD₃OD) δ 7.74 (m, 3H), 7.52 (m, 1H), 7.28 (d, 2H, J=9.5 Hz), 7.08 (dd, 2H, J=9.6, 2.3 Hz), 6.97 (d, 2H, J=2.2 Hz), 3.69 (q, 8H, J=7.1 Hz), 3.39 (m, 8H), 2.37 (m, 2H), 2.30 (m, 2H), 1.59 (m, 4H), 1.31 (t, 12H, J=7.0). ¹³C NMR (125 MHz, MeOD) 12.97, 25.71, 25.87, 33.68, 34.66, 42.88, 47.04, 97.50, 115.00, 115.54, 129.06, 131.44, 131.91, 132.43, 133.31, 136.65, 157.35, 159.41, 161.41, 169.69, 174.01, 177.22. HRMS-EI (m/z): [M] calcd for C₃₈H₄₆N₄O₅+H⁺ 639.3541. found 639.3528.

(187): To an Argon flushed flask cooled to 0° C. containing 186 (141 mg, 0.19 mm) and N-hydroxysuccinimide (430 mg, 3.74 mmol, 20 eq) was added dry DCM (12 mL) followed by DCC (386 mg, 1.87 mm, 10 eq). The reaction was complete by TLC (R_{f(product})=0.7, R_{f(2)}=0.4) in 2.5 hrs at which time the solvent was removed by rotary evaporation. The resulting red solid was purified by reversed phase HPLC (HPLC (B 40% to 70% over 30 min, flow=20 mL/min, λ=530 nm, tᵣₑₜₑₙₜᵢₒₙ=24 min) to afford 134 mg of product (84%). ¹H NMR (300 MHz, CD₃OD) δ 7.74 (m, 3H), 7.53 (m, 1H), 7.28 (d, 2H, J=9.5 Hz), 7.08 (dd, 2H, J=9.5, 2.2 Hz), 6.97 (d, 2H, J=2 Hz) 3.69 (q, 8H, J=7.0 Hz), 3.39 (m, 8H), 2.82 (s, 4H), 2.65 (m, 2H), 2.37 (t, 2H, J=7 Hz), 1.69 (m, 4H), 1.31 (t, 12H, J=7.0). HRMS-EI (m/z): [M] calcd for C₄₂H₅₀N₅O₇⁺ 736.3705. found 736.3719.

(188): 187 (29.1 mg, 0.0377 mmol) was added to a solution of 9 (36.5 mg, 0.0957 mmol) in methanol (1 mL). NMM (80 μL, 0.728 mmol) was added and the reaction was stirred at ambient temperature under argon overnight. Solvent was removed in vacuo. The crude product was purified by HPLC (2:3 95% acetonitrile: 4.9% water: 0.1% TFA: 99.9% water: 0.1% TFA to 55:45% acetonitrile: 4.9% water: 0.1% TFA: 99.9% water: 0.1% TFA over 30 minutes at a flow rate of 20 mL/min). Detection was at 525 nm. The product containing fractions were combined and the solvent was removed by lyophilizer to yield 170 (16.3 mg, 49%). ¹H-NMR (500 MHz, CD₃OD) δ 7.75 (m, 2H), 7.69 (m, 1H), 7.51 (m, 1H), 7.26 (m, 2H), 7.05 (dd, J=9.5 Hz, 2H), 6.95 (s, 2H), 3.67 (q, J=7 Hz, 8H), 3.40 (m, 8H), 3.15 (m, 2H), 2.35 (m, 2H), 2.27 (m, 2H), 1.57 (bs, 4H), 1.29 (t, J=7 Hz, 12H).

(189): 188 (36.6 mg, 0.0412 mmol), NHS (113.2 mg, 0.984 mmol) and DCC (55.8 mg, 0.270 mmol) were combined in a round bottom flask and dissolved in methylene chloride (4.5 mL). The reaction was stirred at ambient temperature for 3 days. Solvent was removed in vacuo and the crude product was purified by HPLC (Synergi RP-Polar, 4 micron, 250×21.2 mm; 2:3 95% acetonitrile: 4.9% water: 0.1% TFA: 99.9% water: 0.1% TFA to 55:45% acetonitrile: 4.9% water: 0.1% TFA: 99.9% water: 0.1% TFA over 30 minutes at a flow rate of 20 mL/min. Detection was at 525 nm. The product was collected at 10 minutes. Product containing fractions were combined and solvent was removed by lyophilizer to yield 171 (21.2 mg, 53%). ¹H-NMR (500 MHz, CD₃OD) δ 7.75 (m, 2H), 7.69 (m, 1H), 7.49 (m, 1H), 7.26 (m, 2H), 7.11 (m, 1H), 7.06 (m, 1H), 6.94 (s, 2H), 4.58 (bs, 1H), 3.67 (q, J=7 Hz, 8H), 3.40 (m, 8H), 3.15 (m, 2H), 2.33 (m, 2H), 2.27 (m, 4H), 1.57 (bs, 4H), 1.50 (m, 2H), 1.29 (t, J=7 Hz, 12H).

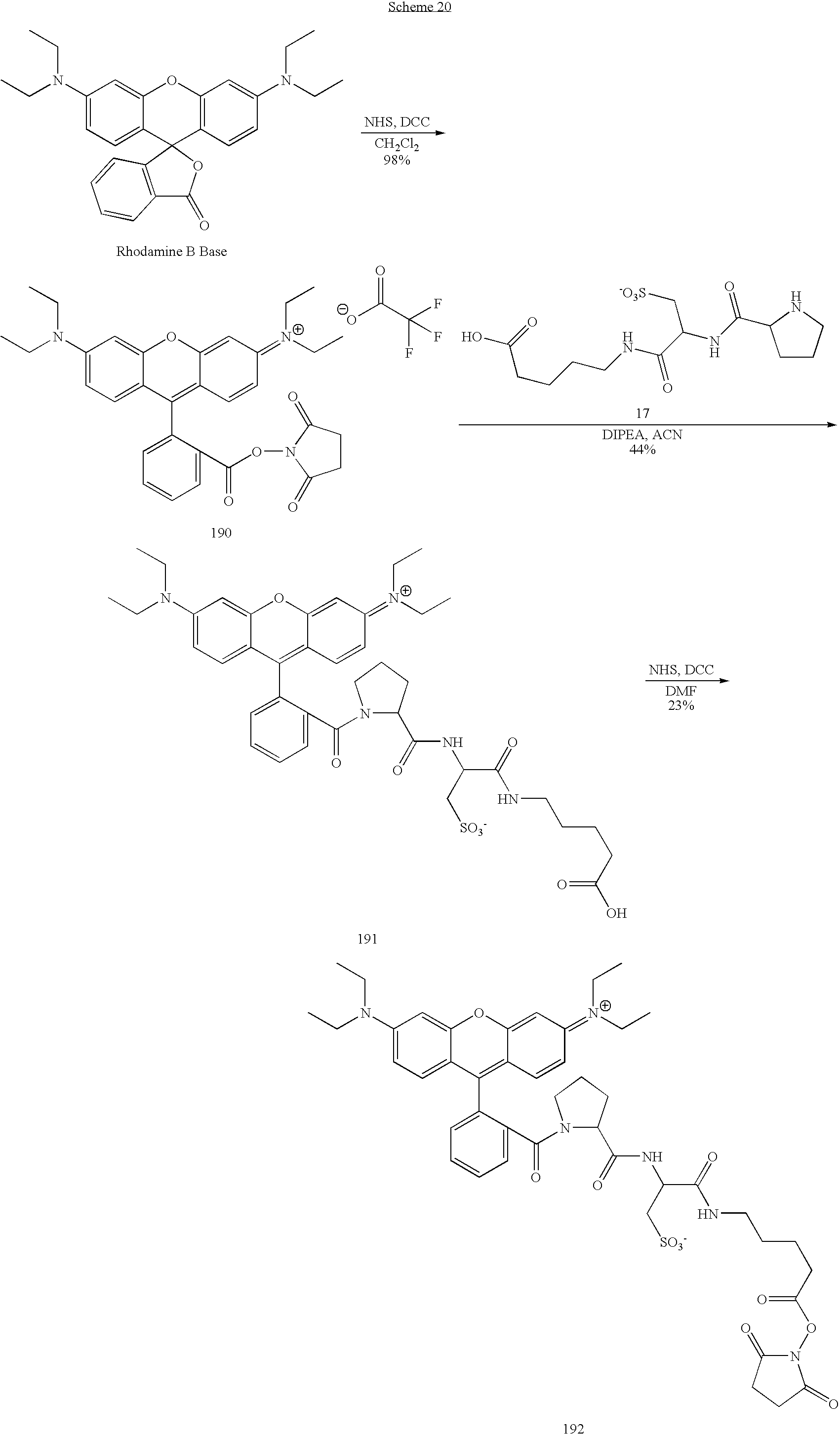

(190): Rhodamine B Base (98.9 mg, 0.206 mmol), NHS (268.4 mg, 1.30 mmol) and DCC (199.5 mg, 1.73 mmol) were combined in a round bottom flask and flushed with argon. This mixture was suspended in methylene chloride (10 mL) and stirred at ambient temperature under argon overnight. Solvent was removed in vacuo and the crude product was purified by HPLC (Synergi RP-Polar, 4 micron, 250×21.2 mm; 45:55 95% acetonitrile: 4.9% water: 0.1% TFA: 99.9% water: 0.1% TFA to 3:1 95% acetonitrile: 4.9% water: 0.1% TFA: 99.9% water: 0.1% TFA over 30 minutes at a flow rate of 20 mL/min. Detection was at 525 nm. The product containing fractions were combined and solvent was removed by lyophilizer to yield 190 (131.8 mg, 98%). ¹H-NMR (500 MHz, CD₃OD) δ 8.42 (d, J=8 Hz, 1H), 8.00 (t, J=7.5 Hz, 1H), 7.90 (t, J=7.5 Hz, 1H), 7.57 (d, J=7.5 Hz, 1H), 7.13 (d, J=9.5 Hz, 2H), 7.03 (dd, J=9.5 Hz, J=2.5 Hz, 2H), 6.95 (d, J=2.5 Hz, 2H), 3.65 (q, J=7 Hz, 8H), 2.84 (s, 4H), 1.29 (t, J=7 Hz, 12H).

(191): 190 (74.0 mg, 0.126 mmol) was combined with side chain 15 in a round bottom flask and flushed with argon. The mixture was suspended in acetonitrile (12 mL) and treated with DIPEA (150 μL, 0.861 mmol). The reaction was stirred at 80° C. for 3 h. Solvent was removed in vacuo and the crude product was purified by HPLC (Synergi RP-Polar, 4 micron, 250×21.2 mm; 2:3 95% acetonitrile: 4.9% water: 0.1% TFA: 99.9% water: 0.1% TFA to 1:1 95% acetonitrile: 4.9% water: 0.1% TFA: 99.9% water: 0.1% TFA over 20 minutes the 100% 95% acetonitrile: 4.9% water: 0.1% TFA: 99.9% water: 0.1% TFA over 10 minutes at a flow rate of 20 mL/min). Detection was at 525 nm. The product containing fractions (25 min) were combined and solvent was removed by lyophilizer to yield 191 (25.6 mg, 44%). ¹H-NMR (500 MHz, CD₃OD) δ 8.08 (d, J=8 Hz, 1H), 7.75-7.77 (m, 2H), 7.47 (d, J=6.5 Hz, 1H), 7.29 (d, J=9.5 Hz, 2H), 7.20 (d, J=9.5 Hz, 1H), 7.05-7.09 (m, 2H), 6.95 (dd, J=9.5 Hz, J=2 Hz, 2H), 4.38 (m, 1H), 3.62-3.87 (m, 10H), 3.42 (t, J=1.5 Hz, 1H), 3.14-3.18 (m, 3H), 2.97-3.01 (m, 2H), 2.76 (m, 1H), 2.18 (m, 1H), 2.04 (t, J=2 Hz, 2H), 2.00 (t, J=2 Hz, 2H), 1.72 (m, 1H), 1.29 (t, J=7 Hz, 12H), 1.14 (m, 2H).

(192): 191 (16.0 mg, 0.0203 mmol), NHS (22.0 mg, 0.191 mmol) and DCC (16.4 mg, 0.0795 mmol) were combined in a round bottom flask and flushed with argon. DMF (2 mL) was added and the reaction was stirred at ambient temperature overnight. Solvent was removed by lyophilizer and the crude product was purified by HPLC (Synergi RP-Polar, 4 micron, 250×21.2 mm; 2:3 95% acetonitrile: 4.9% water: 0.1% TFA: 99.9% water: 0.1% TFA to 55:45 95% acetonitrile: 4.9% water: 0.1% TFA: 99.9% water: 0.1% TFA over 30 minutes at a flow rate of 20 mL/min). Detection was at 525 nm. The product containing fractions (16 min) were combined and solvent was removed by lyophilizer to yield 192 (2.2 mg, 12%).

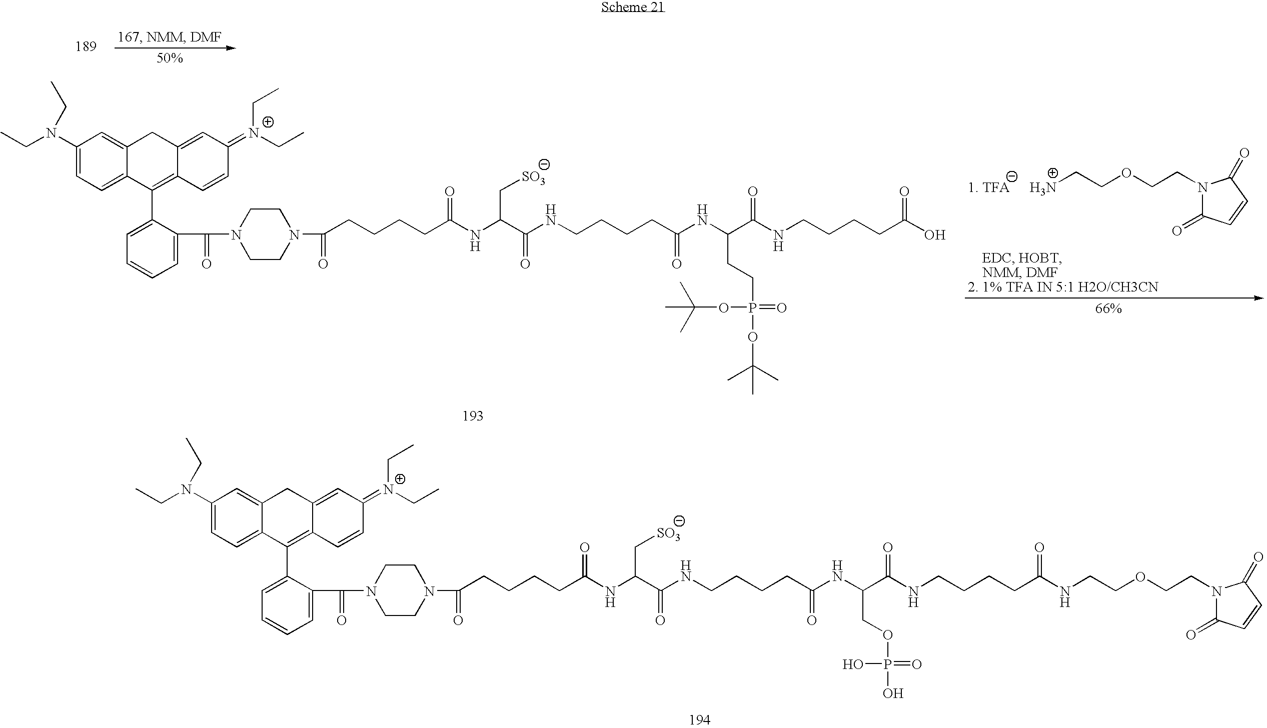

(193): To an Argon flushed flask containing 187 (38 mg, 39 umol) was added a DMF solution of 167 (31 mg, 78 umol, 2 mL) followed by NMM (4.7 uL, 43 umol, 4 eq). The reaction was complete by TLC (R_{f(product)}=0.4, R_{f(6)}=0.7, 3:1 DCM/MeOH) after 21 hours at which time the solvent was removed in vacuo. The resulting red solid was purified by RP HPLC (B 20% to 70% over 30 min, flow=20 mL/min, λ=530 nm, tᵣₑₜₑₙₜᵢₒₙ=23 min) to afford 24 mg product (50%). ¹H NMR (300 MHz, CD₃OD) δ 7.75 (m, 3H), 7.50 (m, 1H), 7.29 (d, 2H, J=9.5 Hz), 7.1 (m, 1H), 6.97 (s, 2H), 4.58 (m, 2H), 4.16 (m, 2H), 3.69 (q, 8H, J=6.9 Hz), 3.39 (m, 8H), 3.18 (m, 6H), 2.31 (m, 8H), 1.59 (m, 12H), 1.48 (m, 18H), 1.30 (t, 12H, J=7 Hz). ³¹P NMR (125 MHz, CD₃OD) δ −11.64 vs 85% H₃PO₄. HRMS-EI (m/z): [M] calcd C₆₂H₉₁N₈O₁₆PSH+Na⁺+Na⁺ 656.2898. found 656.2857.

(194): To an Argon flushed flask containing 193 (3.3 mg, 2.7 umol), HOBt (1.5 mg, 10.8 umol, 4 eq), and 172 (3.24 mg, 10.8 umol, 4 eq), was added sequentially dry DMF (0.5 mL), NMM (2.4 uL, 21.6 umol, 8 eq), and EDC (2.1 mg, 10.8 umol, 4 eq). The solution was stirred under Argon for 21 hours at which time the solvent was removed in vacuo. To the resulting red solid was added a solution of 1% TFA in 5:1 H₂O/CH₃CN (0.6 mL) and the solution was stirred under Argon for 24 hours. The solvent was removed in vacuo followed by purification by RP HPLC (B 30% to 70% over 30 min, flow=4 mL/min, 530 nm, tᵣₑₜₑₙₜᵢₒₙ=14.4 min) to afford 2.6 mg product (66%). ¹H NMR (500 MHz, CD₃OD) δ 7.75 (m, 3H), 7.51 (m, 1H), 7.29 (d, 2H, J=9.1 Hz), 7.10 (m, 2H), 6.97 (s, 2H), 6.83 (s, 2H), 4.60 (m, 2H), 4.17 (m, 2H), 3.69 (m, 10H), 3.59 (t, 2H, J=5.4 Hz), 3.49-3.39 (m, 10H), 3.22 (m, 6H), 2.37-2.2 (m, 8H), 1.57 (m 12H), 1.31 (t, 12H, J=6.9 Hz). ³¹P NMR (125 MHz, CD₃OD) δ −1.27 vs 85% H₃PO₄. HRMS-EI (m/z): [M] calcd for C₆₂H₈₃N₁₀O₁₈PS⁻² 659.2678. found 659.2659.

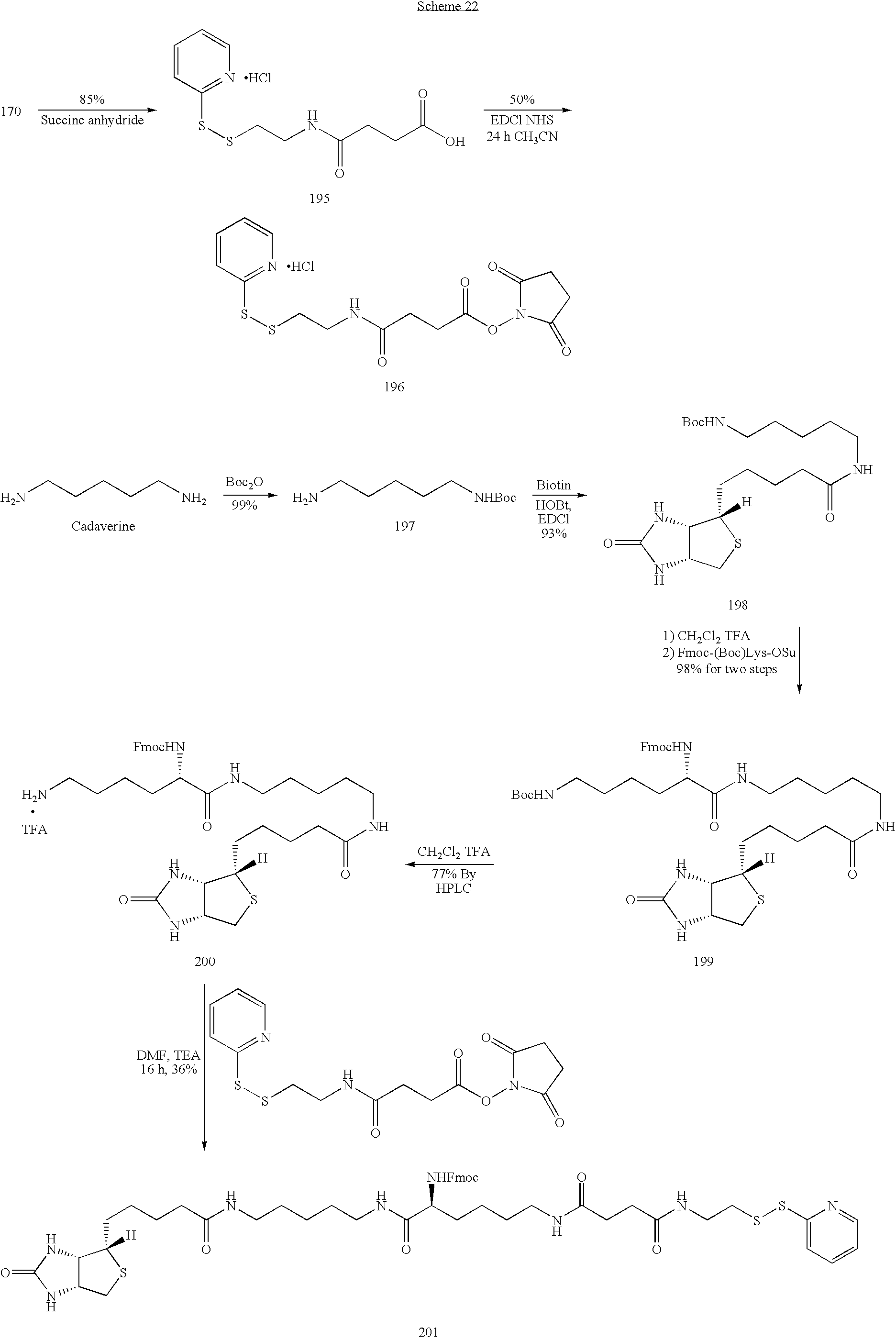

195: A solution of 170 (50 mg, 0.22 mmol), Succinic anhydride (22 mg, 0.22 mmol), potassium carbonate (35 mg, 0.25 mmol) in acetonitrile 3.0 mL was stirred overnight under argon at room temperature. Solvent was removed under reduced pressure and the resulting residue was acidified to pH=2˜3 by using 2 N hydrochloride. The crude product was separated by HPLC yielding 60 mg (85%) of 195 as a white solid. ¹H NMR (MeOD, 500M) δ 8.45 (m, 1H); 7.90 (m, 2H); 7.31 (m, 1H); 3.46 (t, J=6.5, 2H); 2.93 (t, J=6.5, 2H); 2.56 (t, J=7.0, 2H); 2.44 (t, J=7.0, 2H). MS (Microtof) 287.0505 (M+1)⁺

196: A solution of 195 (50 mg, 0.17 mmol), EDCI (40 mg, 0.21 mmol), NHS (25 mg, 0.21 mmol) in acetonitrile 2.0 mL was stirred overnight under argon at room temperature. Solvent was removed under reduced pressure and the resulting residue was separated by HPLC yielding 25 mg (50%) of 196 as a white solid, also 12 mg of 195 was recovered. ¹H NMR (MeOD, 500M) δ 8.45 (m, 1H); 7.90 (m, 2H); 7.31 (m, 1H); 3.46 (t, J=6.5, 2H); 2.93 (m, 4H); 2.85 (s, 4H); 2.56 (t, J=6.5, 2H).

197: Di-tert-butyl dicarbonate (1.1 g, 5.5 mmol) in 50 mL of 9:1 dioxane/water was added to a solution of 1,5-diaminopentane (2.5 mL, 21 mmol) in 50 mL of 9:1 dioxane/water over a period of 3.0 hours. The solution was stirred at room temperature overnight and concentrated and the residue was taken up in 50 mL of water. The precipitated N,N′-di-Boc-1,5-diaminopentane was removed by filtration through a fitted glass funnel, and the filtrate was extracted with methylene chloride (50 mL×4). The combined organic extracts were washed by water (20 mL×2), then dried by sodium sulfate. Removing the solvent to yielding 197 1.0 g (99%) as a yellow oil. ¹H NMR (CDCl₃, 500M) δ 4.52 (br, 1H); 3.08 (m, 2H); 2.66 (m, 2H); 1.48 (m, 11H); 1.31 (m, 2H); 1.06 (m, 2H).

198: A solution of 197 (0.9 g, 4.2 mmol), Biotin (0.8 g, 3.3 mmol), EDCI (0.96 g, 5.0 mmol), HOBt (0.68 g, 5.0 mmol), DIPEA 2.0 mL in DMF 8.0 mL was stirred 48 hours under argon at room temperature. Solvent was removed by using a lyophilize and the resulting residue was dissolved in methylene chloride 100 mL and washed by 2.0 N hydrochloride, brine and dried by anhydrous sodium sulfate. After removing the solvent, the crude residue was purified by chromatograph to give 1.3 g (93%) of 198 as a white solid. ¹H NMR (CDCl₃, 500M) δ 4.45 (m, 1H), 4.26m, 1H), 3.12 (m, 3H), 3.00 (t, J=6.0, 2H), 2.88 (dd, J₁=5.0 Hz, J₂=13.0 Hz, 1H), 2.66 (d, J=12.5 Hz, 1H), 2.15 (t, J=7.0 Hz, 2H), 1.60 (m, 4H), 1.48 (m, 15H), 1.28 (m, 2H).

199: A solution of 198 (1.0 g, 2.3 mmol), in 12 mL of 3:1 DCM/TFA was stirred overnight under argon at room temperature. Solvent was removed under reduced pressure and the resulting residue was used to the next step without further purification.

To a stirred solution of Fmoc-(Boc)Lys-OH (1.0 g, 2.1 mmol), in DMF 5.0 mL was added EDCI (530 mg, 2.8 mmol) and NHS (350 mg, 3.0 mmol). The resulting solution was stirred overnight under argon at room temperature. The product was extracted by ethyl acetate (50 mL) and washed by 1 N hydrochloride, saturate aqueous sodium bicarbonate and brine, then, dried by anhydrous sodium sulfate. Solvent was removed under reduced pressure and the resulting residue was used to the next step without further purification.

To a stirred solution of the resulting crude product from step 1 and step 2 in methanol 30 mL, DIPEA 1.5 mL was added. After stirring for 7.0 hours under argon at room temperature, methanol was removed under reduced pressure and the product was extracted with methylene chloride and washed by brine dried by anhydrous sodium sulfate. the crude residue was purified by chromatograph to give 1.6 g (98%) of 199 as a white foam. ¹H NMR (MeOD, 500M) δ 7.79 (m, 2H); 7.63 (m, 2H); 7.35 (m, 2H); 7.28 (m, 2H); 4.43 (m, 1H), 4.36 (m, 2H), 4.20 (m, 2H), 4.0 (m, 1H), 3.12 (m, 5H), 3.00 (t, J=6.5, 2H), 2.88 (dd, J₁=5.0 Hz, J₂=13.0 Hz, 1H), 2.66 (d, J=12.5 Hz, 1H), 2.15 (t, J=7.0 Hz, 2H), 1.60 (m, 6H), 1.47 (m, 6H), 1.40 (m, 12H), 1.31 (m, 3H). MS (Microtof) 801.3952 (M+Na)⁺

200: A solution of 199 (300 mg, 0.4 mmol), in 3 mL of 2:1 DCM/TFA was stirred overnight under argon at room temperature. After removing solvents, the crude product was purified by HPLC yielding 200 mg (77%) of 200 as a white foam. ¹H NMR (MeOD, 500M) δ 7.79 (m, 2H); 7.63 (m, 2H); 7.35 (m, 2H); 7.28 (m, 2H); 4.43 (m, 1H), 4.36 (m, 2H), 4.20 (m, 2H), 4.0 (m, 1H), 3.12 (m, 5H), 2.86 (m, 3H), 2.66 (d, J=12.5 Hz, 1H), 2.15 (t, J=7.0 Hz, 2H), 1.66 (m, 1H), 1.56 (m, 7H), 1.46-1.30 (m, 10H). MS (Microtof) 679.3637 (M+H)⁺

201: To a stirred solution of 200 (30 mg, 44 umol), 196 (26 mg, 68 umol) in DMF 2.0 mL was added triethylamine 0.1 mL. After stirring 16 hours under argon at room temperature, the crude residue was purified by HPLC to give 15 mg (36%) of 201 as a white powder. ¹H NMR (MeOD, 500M) δ 8.60 (m, 1H); 8.25 (m, 1H); 8.15 (m, 1H); 7.78 (m, 2H); 7.63 (m, 3H); 7.35 (m, 2H); 7.28 (m, 2H); 4.45 (m, 1H), 4.35 (m, 2H), 4.25 (m, 1H), 4.18 (m, 1H), 4.0 (m, 1H), 3.45 (m, 2H), 3.14 (m, 7H), 3.0 (m, 2H), 2.87 (m, 1H), 2.66 (d, J=12.5 Hz, 1H), 2.46 (m, 4H), 2.15 (t, J=7.0 Hz, 2H), 1.68-1.30 (m, 18H). MS (Microtof) 947.401 (M+H)⁺.

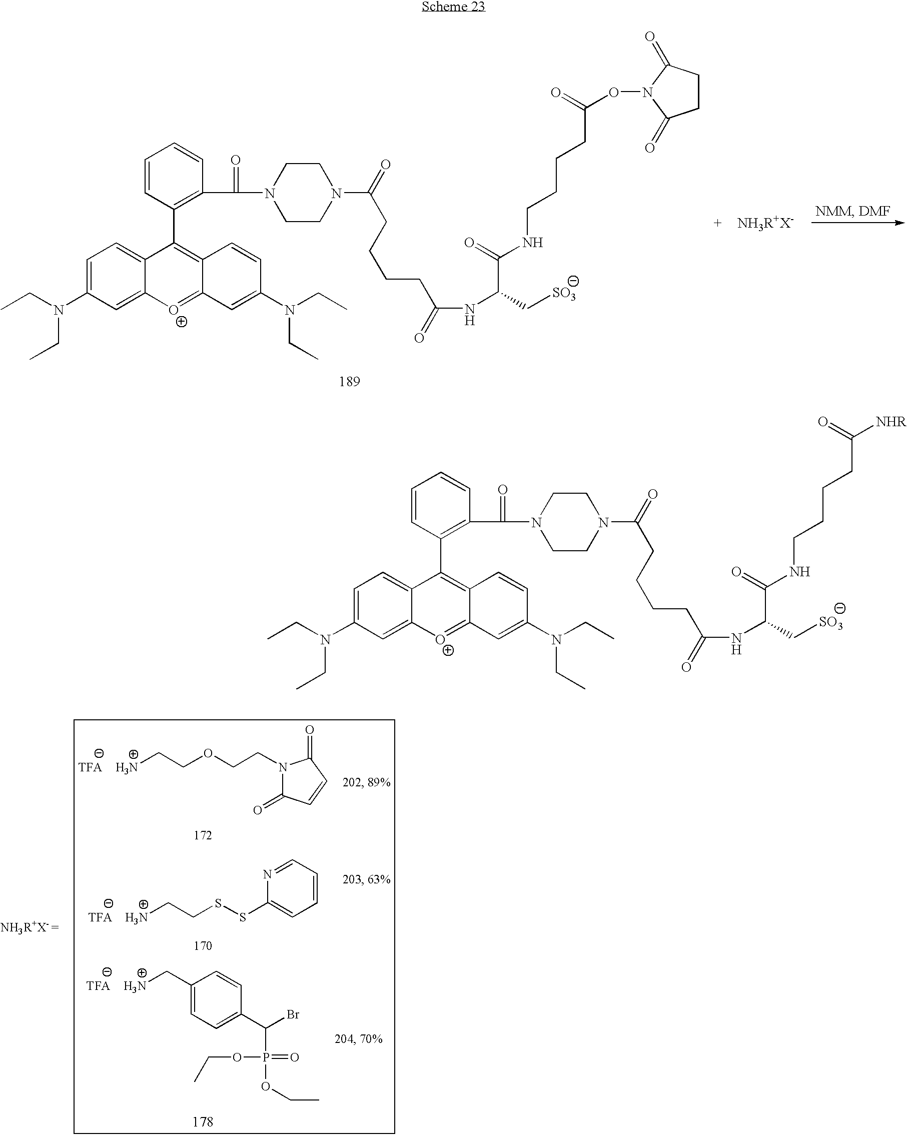

(202): To an Argon flushed flask containing 189 (21 mg, 21 umol) and 172 (13 mg, 42 μmol, 2 eq) was added dry DMF (1 mL) followed by NMM (9.4 uL, 84 μmol, 4 eq). The solution was stirred under Argon for 17 hours monitoring the reaction progress by TLC(R_{f(product)}=0.2, R_{f(6)}=0.4, 6:1 DCM/MeOH). Then the solvent was removed under vacuum and the resulting red solid was purified by reversed phase HPLC (B 40% to 70% over 30 min, flow=20 mL/min, λ=530 nm, tᵣₑₜₑₙₜᵢₒₙ=11 min) to afford 20 mg (89%). ¹H NMR (300 MHz, CD₃OD) 7.75 (m, 3H), 7.50 (m, 1H), 7.29 (d, 2H, J=9.5 Hz), 7.1 (m 2H), 6.96 (s, 2H), 6.82 (s, 2H), 4.60 (m, 1H), 3.68 (m, 10H), 3.58 (t, 2H, 5.3 Hz), 3.49 (t, 2H, 5.4 Hz), 3.28 (m, 2H), 3.17 (m, 4H), 2.35 (m, 6H), 1.60 (m, 8H), 1.30 (t, 12H, J=7 Hz). HRMS-EI (m/z): [M] calcd C₅₄H₇₀N₈O₁₂S+2Na⁺ 550.2309. found 550.2260.

(203): To an argon flushed flask containing 189 (20 mg, 20.3 μmol) and 170 (9 mg, 40.6 μmol, 2 eq) was added DMF (1 mL), followed by NMM (9 uL, 81.2 μmol, 4 eq). The resulting solution was stirred for 18 hours at which time the solvent was removed by vacuum. The resulting red solid was purified by RP HPLC (B 30% to 70% over 30 min, flow=20 mL/min, λ=530 nm, tᵣₑₜₑₙₜᵢₒₙ=18 min) to afford 13.6 mg (63%). ¹H NMR (300 MHz, CD₃OD) δ 8.44 (d, 1H, J=4.2 Hz), 7.91 (m, 2H), 7.73 (m, 3H), 7.48 (m, 1H), 7.33-7.25 (m, 3H), 7.06 (m, 2H), 6.93 (m, 2H), 4.57 (m, 1H), 3.68 (q, 8H, J=7 Hz), 3.51-3.42 (m, 10H), 3.16 (m, 4H), 2.94 (t, 2H, J=6.5 Hz), 2.32-2.13 (m, 6H), 1.59-1.50 (m, 6H), 1.28 (t, 12H, J=6.9 Hz). HRMS-EI (m/z): [M] calcd C₅₃H₆₈N₈O₉S₃+H⁺Na⁺ 540.2118. found 540.2115.

(204): To an argon flushed flask containing 189 (20 mg, 20.3 μmol) and 178 (24 mg, 56.8 μmol, 2.8 eq) was added DMF (1 mL) followed by NMM (12.5 uL, 114 μmol, 5.6 eq). The resulting solution was stirred under argon for 24 hours at which time the solvent was removed by vacuum. The resulting red solid was purified by RP HPLC (B 30% to 70% over 30 min, flow=20 mL/min, λ=530 nm, tᵣₑₜₑₙₜᵢₒₙ=20 min) to afford 17.2 mg (70%). ¹H NMR (500 MHz, CD₃OD) δ 7.77-7.71 (m, 3H), 7.54-7.50 (m, 3H), 7.27 (m, 4H), 7.20-7.01 (m, 2H), 6.96 (m, 2H), 5.27 (d, 1H, J=13 Hz), 4.60 (m, 1H), 4.35 (s, 2H), 4.22-4.19 (m, 2H), is 4.00 (m, 1H), 3.88 (m, 1H), 3.69 (q, 8H, J=7 Hz), 3.53 (m, 1H), 3.37 (m, 7H), 3.16 (m, 4H), 2.34-2.23 (m, 6H), 1.64-1.52 (m, 8H), 1.34-1.29 (m, 15H), 1.14 (t, 3H, J=7 Hz). [M] calcd C₅₈H₇₇N₇O₁₂S+[H⁺]+[H⁺] 603.7209. found 603.7029.

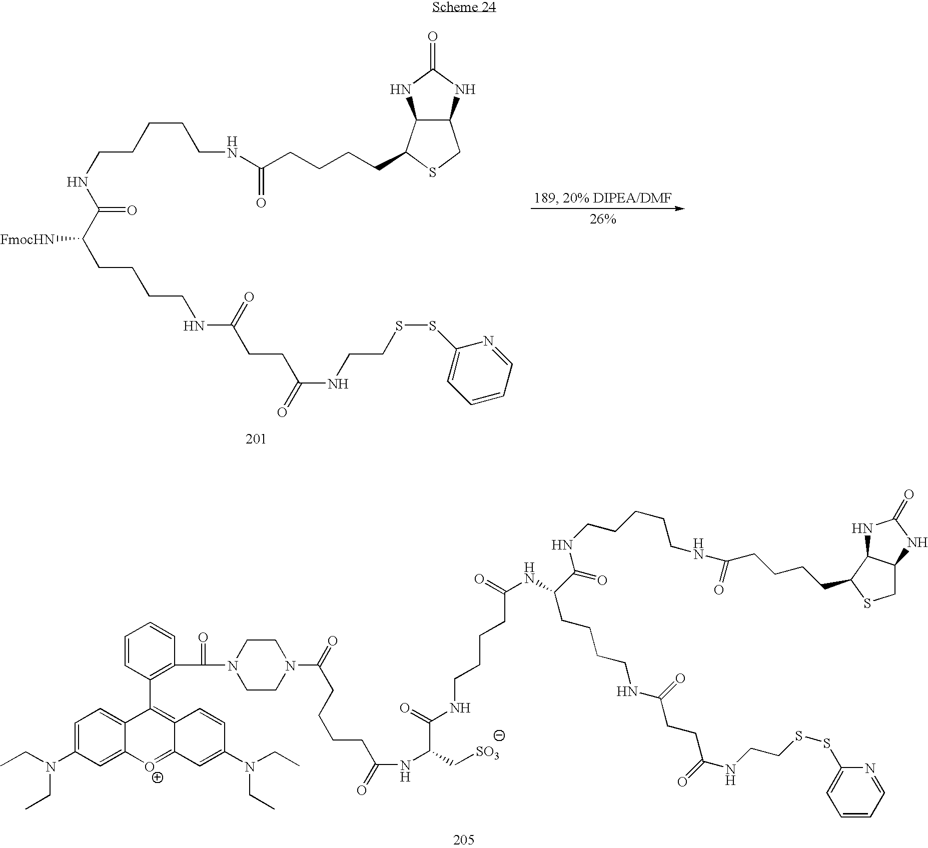

(205): To an argon flushed flask containing 189 (15 mg, 15.8 μmol) and 201 (15 mg, 15.8 μmol) was added DMF (0.8 mL) followed by DIPEA (0.2 mL). The resulting solution was stirred for 4 days at which time the solvent was removed by vacuum. The resulting red solid was purified by RP HPLC (B 40% to 70% over 30 min, flow=20 mL/min, λ=530 nm, tᵣₑₜₑₙₜᵢₒₙ9=8.7 min) to afford 6.7 mg (26%, not optimized). ¹H NMR (500 MHz, CD₃OD) δ 8.42 (d, 1H, J=4.9 Hz), 7.87 (m, 2H), 7.76-7.70 (m, 3H), 7.48 (m, 1H), 7.25 (d, 2H, J=9.5 Hz), 7.06 (m, 2H), 6.93 (m 2H), 4.60 (m, 1H), 4.45 (m, 1H), 4.27 (m, 1H), 4.18 (m, 1H), 3.64 (q, 8H, J=7 Hz), 3.46-3.42 (m, 10H), 3.13 (m, 11H), 2.94-2.86 (m, 3H), 2.66 (d, 1H, J=12.8 Hz), 2.44 (s, 4H), 2.33-2.14 (m, 8H), 1.90-1.40 (m, 26H), 1.27 (t, 12H, J=7 Hz). [M] calcd C₇₈H₁₁₀N₁₄O₁₄S+[Na⁺]+[Na⁺] 820.3496. found 820.3494.

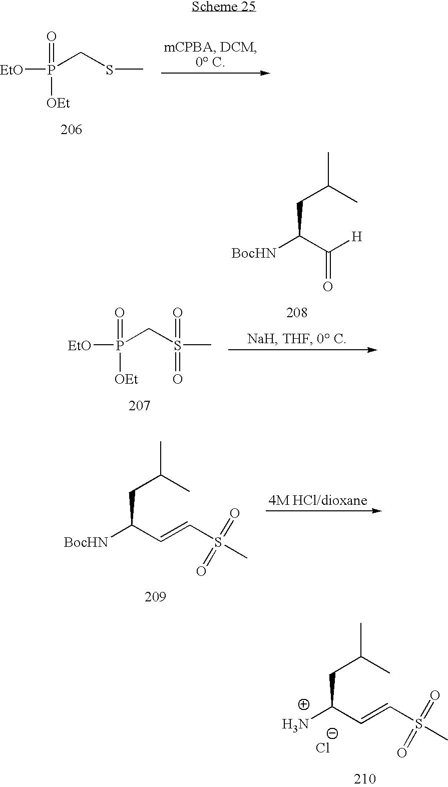

(207): To an argon flushed flask containing 206 (5 g, 25.2 mmol) at 0° C. was added DCM (80 mL) followed by mCPBA (77%, 12.4 g, 2.2 eq). The solution was stirred under argon at 0° C. for 22 hours at which time the resulting solid was filtered and washed with DCM (100 mL). The organic solution was then washed with 3M NaOH three times followed by one wash with brine. After drying over Na₂SO₄, the solvent was removed by vacuum to provide 5.13 g of white solid (88%). ¹H NMR (500 MHz, CDCl₃) δ 4.24 (q, 4H, J=7 Hz), 3.59 (d, 2H, J=16 Hz), 3.22 (s, 3H), 1.38 (t, 6H, J=7 Hz).

(209): NaH (95% in oil, 10.4 mg, 1.25 eq) was added to a THF solution (4 mL) of 207 (84 mg, 0.36 mmol, 1.1 eq) at 0° C. under argon. Then a THF solution (1 mL) of 208 (71 mg, 0.33 mmol, 1 eq) was added dropwise and the solution was stirred for 90 minutes. Then 1 M HCl (1 mL) was added followed by 2 mL EtOAc. The organic layer was washed sequentially with 1 M HCl, saturated NaHCO_{3(aq)}, and brine. After drying (Na₂SO₄) the solvent was removed and the resulting white solid was purified by column chromatography (Premium Rf silica gel, 4:1 Hexanes/EtOAc, R_{f(product)}=0.1) to afford 72 mg (74%). ¹H NMR (300 MHz, CDCl₃) δ 6.82 (dd, 1H, J=3.5, 15 Hz), 6.48 (d, J=15 Hz), 4.46 (m, 2H), 2.94 (s, 3H), 1.73-1.6 (m, 3H), 1.46 (s, 9H) 0.96-0.94 (m, 6H).

(210): 4 M HCl (0.5 mL) in dioxane was added to a flask containing 209 (48 mg, 0.163 mmol) and the resulting solution was stirred under argon for 1 hour. Then Et₂O (1 mL) was added and the organic layer was washed with water. The aqueous layers were combined and brought to dryness under vacuum to afford 30.8 mg of white solid (78%). ¹H NMR (300 MHz, CDCl₃) δ 7.03 (d, 1H, J=15.3 Hz), 6.74 (dd, 1H, J=7.7, 15.3 Hz). 4.06 (m, 1H), 3.03 (s, 3H), 1.63 (m, 3H), 0.98 (m, 6H).

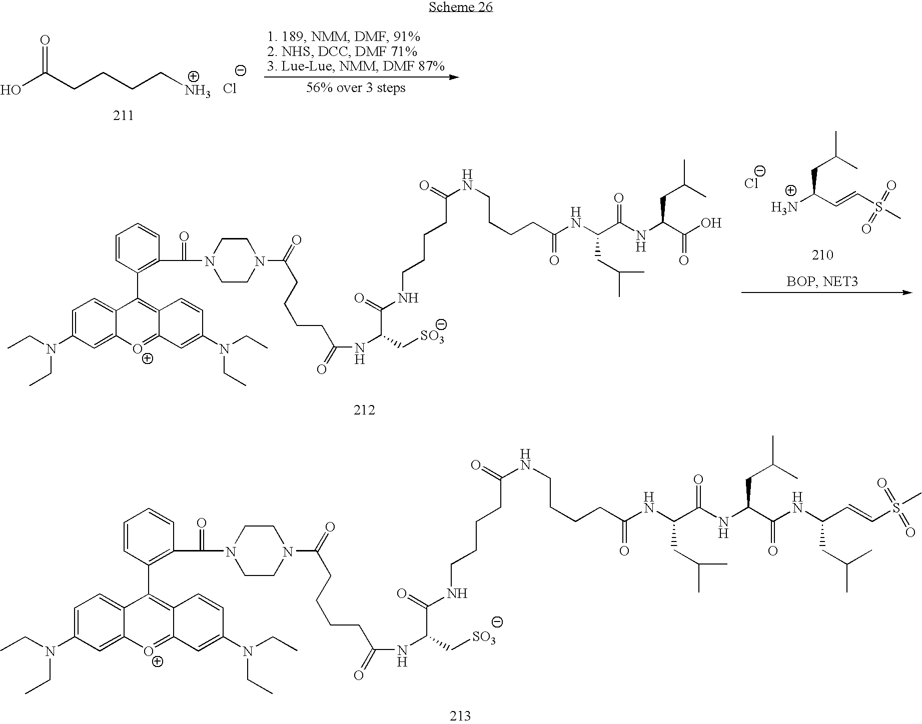

(212): To an argon flushed flask containing 211 (40 mg, 40.6 μmol) and 189 (25 mg, 4 eq) was added DMF (2 mL) followed by NMM (8 μL, 8 eq). The reaction was followed by TLC (4:1 DCM/MeOH R_{f(1)}=0.7, R_{f(product)}=0.25). After 24 hours stirring under argon, the solvent was removed by vacuum. The resulting red solid was purified by RP HPLC (B 30% to 70%=over 30 min, flow=20 mL/min, λ=530 nm, tᵣₑₜₑₙₜᵢₒₙ=16 min) to afford 36.4 mg (91%). ¹H NMR (300 MHz, CD₃OD) δ 7.74-7.69 (m, 3H), 7.47 (m, 1H), 7.25 (d, J=9.4 Hz), 4.57 (m, 1H), 3.65 (q, 8H, J=6.7 Hz), 3.50 (m, 1H), 3.35 (m, 7H), 3.14 (m, 6H), 2.32-2.11 (m, 8H, 1.58-1.49 (m, 12H), 1.27 (t, 12H, J=6.8 Hz).

The resulting acid (36.4 mg, 36.8 umol) was added to an argon flushed flask containing DCC (76 mg, 368 umol, 10 eq) and NHS (85 mg, 736 mm, 20 eq) which was then dissolved by DMF (2 mL) and stirred under argon. The reaction was followed by TLC (8:1 DCM/MeOH, R_{f(SM)}=0.13, R_{f(product)}=0.63) and after 24 hours the solvent was removed by vacuum. The resulting red solid was purified by RP HPLC (B 30% to 70% over 30 min, flow=20 mL/min, λ=530 nm, tᵣₑₜₑₙₜᵢₒₙ=16.5 min) to afford 28.3 mg (71%). ¹H NMR (500 MHz, CD₃OD) δ 7.69 (m, 3H), 7.46 (d, 1H, J=6.1 Hz), 7.25 (d, 2H, J=9 Hz), 7.08-7.03 (m, 2H), 6.91 (m, 2H), 4.54 (m, 2H), 3.64 (q, 8H, J=7 Hz), 3.49 (m, 1H), 3.33-3.25 (m, 7H), 3.11 (m, 6H), 2.76 (s, 4H), 2.60 (t, 2H, J=7 Hz), 2.30-2.20 (m, 4H), 2.11 (m, 2H), 1.88-1.44 (m, 12H), 1.24 (t, 12H, J=7).

The succinimide (26.3 mg, 24.2 umol) was then added to an argon flushed flask containing Leu-Leu-OH (24 mg, 97 umol, 4 eq) and dissolved in DMF (2 mL). Then NMM (21 uL, 194 umol, 8 eq) was added dropwise and the solution was stirred under argon for 20 hours at which time the solvent was removed by vacuum. The resulting red solid was purified by RP HPLC (B 30% to 70% over 30 min, flow=20 mL/min, λ=530 nm, tᵣₑₜₑₙₜᵢₒₙ=18 min) to afford 26.5 mg of 212 (87%). ¹H NMR (500 MHz, CD₃OD) δ δ 7.69 (m, 3H), 7.46 (d, 1H, J=6.4 Hz), 7.25 (d, 2H, J=9 Hz), 7.09-7.04 (m, 2H), 6.92 (m, 2H), 4.54 (m, 2H), 4.38 (m, 2H), 3.64 (t, 8H, J=7), 3.50 (m, 1H), 3.35 (m, 7H), 3.12 (m, 6H), 2.32-2.13 (m, 8H), 1.63-1.57 (m, 18H), 1.26 (t, 12H, J=6.7 Hz), 0.92-0.91 (m, 12H). [M] calcd C₆₃H₉₁N₉O₁₃S+[Na⁺] 1236.6349. found 1236.6362.

(213): 212 (22.5 mg, 18.5 umol) was added to an argon flushed flask at 0° C. and dissolved in DMF (1 mL) followed by the addition of DIPEA (8 uL, 2.1 eq). Then BOP (8.2 mg, 1 eq) was added followed by 210 (5 mg, 1.1 eq) and the solution was stirred for 2 hours at 0° C. under argon. The solvent was then removed by vacuum and the resulting solid was purified by RP HPLC (B 30% to 70% over 30 min, flow=20 mL/min, λ=530 nm, tᵣₑₜₑₙₜᵢₒₙ=21.4 min) to afford 3.2 mg of 213 (13%, not optimized). ¹H NMR (500 MHz, CD₃OD) δ 8.02 (m, 1H), 7.74 (m, 3H), 7.50 (m, 1H), 7.27 (d, 2H, J=9 Hz), 7.08 (m, 2H), 6.95 (m, 2H), 6.77 (dd, 1H, J=4.9, 15 Hz), 6.60 (d, 1H, J=15 Hz), 4.62 (m, 2H), 4.31 (m, 2H), 3.65 (t, 8H, J=7 Hz), 3.37 (m, 1H), 3.39 (m, 7H), 3.15 (m, 6H), 2.96 (s, 3H), 2.34-2.13 (m, 8H), 1.61-1.42 (m, 21H), 1.31-1.26 (t, 12H, J=7 Hz), 0.96-0.89 (m, 18H). [M] calcd C₇₁H₁₀₆N₁₀O₁₄S₂+ [Na⁺] 1409.7224. found 1409.7257.

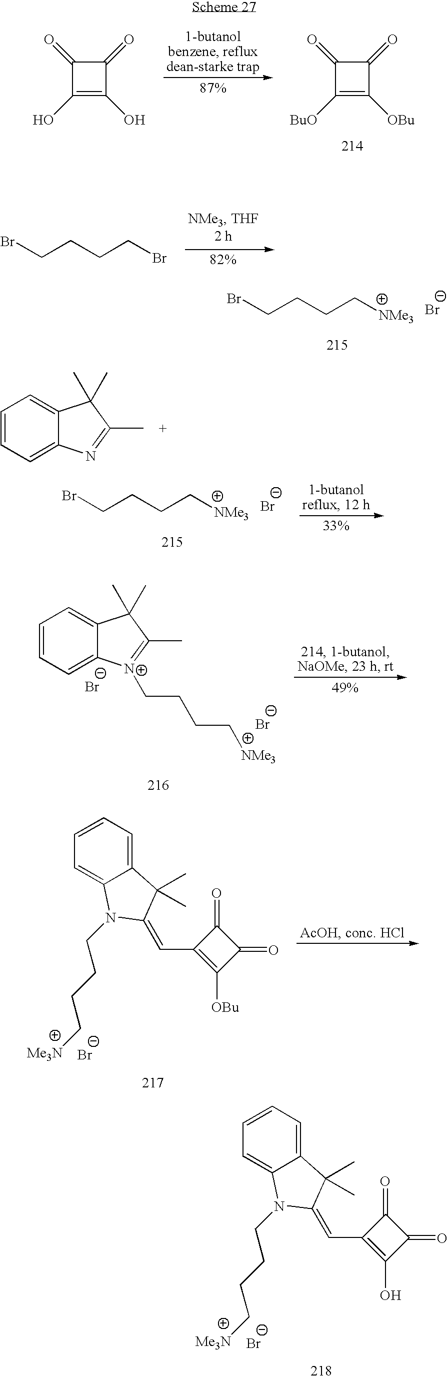

3,4-Di-n-butyl-cyclobut-3-en-1,2-dione (214): A suspension of 3,4-Dihydroxy-3-cyclobutene-1,2-dione (10 g, 87.7 mmol) in 100 ml 1-butanol (1.1 ml/mmol) and 10 ml benzene (0.11 ml/mmol) was refluxed using a Dean-Starke-Trap to remove water. Once the mixture became a clear solution and the theoretical amount of water was collected evaporation of the solvent followed. The resulting yellow oil was subjected to flash chromatography through a short column of silica gel (2/1 hexanes/ethyl acetate). The solvent was removed under reduced pressure to afford 214 (17.3 g, 76.5 mmol, 87%) as a clear oil.

(215): Through a solution of 1,4-dibromobutane (10 g, 46.31 mmol, 5.5 ml) in 96 ml THF (2 ml/mmol) was bubbled gaseous trimethyl amine for 2 h at room temperature. The resulting white precipitate was filtered off and washed with diethyl ether to give 215 (10.4 g, 38 mmol, 82%). ¹H NMR (300 MHz, CD₃CN) δ 1.80-1.92 (m, 4H), 3.05 (s, 9H), 3.27-3.38 (m, 2H), 3.47-3.55 (m, 2H).

(216): To a solution of 2,3,3-Trimethylindolenine (2.7 g, 17 mmol, 1.2 eq.) in 20 ml 1-butanol (1.2 ml/mmol) was added bromide 215 (3.89 g, 14.1 mmol, 1 eq.) and the resulting suspension refluxed overnight. The mixture was then treated with ethyl acetate and the precipitate filtered off to give 216 (2.04 g, 4.7 mmol, 33%). ¹H NMR (300 MHz, DMSO) δ 1.50 (s, 6H), 1.75-1.90 (m, 4H), 2.81 (s, 3H), 3.05 (s, 9H), 3.26-3.40 (m, 2H), 4.40-4.54 (m, 2H), 7.58-7.62 (m, 2H), 7.77-7.83 (m, 2H), 7.95-8.05 (m, 2H).

(217): To a suspension of 114 (2.2 g, 4.16 mmol, 1 eq.) in 16 ml 1-butanol (4 ml/mmol) was added sodium methoxide (216 mg, 4 mmol, 1.2 eq) and stirred for 15 min at room temperature. To this was added a solution of 116 (987 mg, 4.36 mmol, 1.3 eq.) in 1 ml 1-butanol (0.25 ml/mmol). Stirring was continued for 23 h whereas the color changed to a yellowish suspension. The product 217 has a Rf value of 0.3 (3/1 CHCl₃/MeOH) and appears as a yellow spot on TLC. The reaction mixture was quenched with water, followed by extraction with CH₂Cl₂ until the extract was colorless. Drying of the organic phase with MgSO₄ and evaporation of the solvent gave a dark brown residue which was purified by flash chromatography through a short column of silica gel (5/1 to 1/1 CHCl₃/MeOH) to give 217 (796 mg, 1.57 mmol, 49%). ¹H NMR (300 MHz, CDCl₃) δ 0.98 (t, J=7.4 Hz, 3H), 1.00-1.10 (m, 2H), 1.55 (s, 6H), 1.62-1.75 (m, 2H), 1.79-2.10 (m, 6H), 3.49 (s, 9H), 3.78-3.88 (m, 2H), 3.95-4.05 (m, 2H), 4.85-4.92 (m, 2H), 5.42 (s, 1H), 7.00-7.40 (m, 4H).

(218): To a solution of 217 (796 mg, 1.57 mmol) in 10 ml acetic acid was added 2 ml concentrated hydrochloric acid (12 M). The yellow solution was slightly heated with an oil bath until TLC showed the consumption of the starting material (after approximately 5 min). The product has a R_{f} value of 0.5 (TLC C₁₈ 2/1 MeOH/H₂O) and appears as a yellow spot on reversed phase TLC. Evaporating under reduced pressure gave 218 which was used without further purification. ¹H NMR (300 MHz, MeOD) δ 1.59 (s, 6H), 1.65-1.75 (m, 2H), 1.80-2.0 (m, 2H), 3.10 (s, 9H), 3.30-3.45 (m, 2H), 3.85-4.05 (m, 2H), 6.93-7.05 (m, 2H), 7.21-7.45 (m, 2H), no signal for double bond due to exchange.

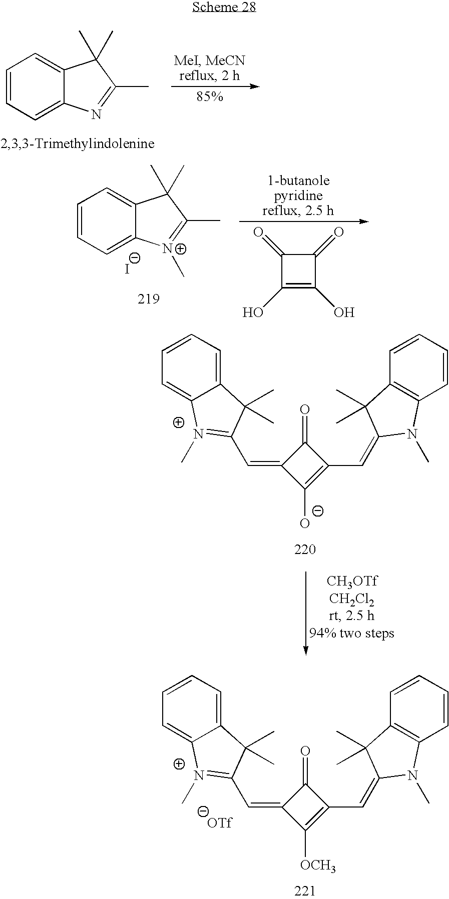

1,2,3,3-Tetramethyl-3H-indolenium iodide (219): To a solution of 2,3,3-Trimethylindolenine (3.7 g, 23.2 mmol, 3.7 ml) in 15 ml dry acetonitrile (0.7 ml/mmol) was added methyl iodide (3.63 g, 25.6 mmol, 1.6 ml, 1.1 eq.) at room temperature. The solution was refluxed for 2 h and then cooled with an icebath. The pink precipitate was filtered off and washed with acetonitrile to yield 219 (5.94 g, 19.7 mmol, 85%). ¹H NMR (300 MHz, D₂O) δ 1.45 (s, 6H), 2.67 (s, 3H), 3.90 (s, 3H), 7.51-7.65 (m, 4H).

(220): To a solution of 1,2,3,3-Tetramethyl-3H-indolenium iodide (219) (3.01 g, 10 mmol, 2 eq.) in 40 ml 1-Butanol and 10 ml pyridine was added 3,4-Dihydroxy-3-cyclobutene-1,2-dione (570 mg, 5 mmol, 1 eq.). The mixture was refluxed for 2.5 h and then cooled to 0° C. The precipitate was filtered off and washed with 1-butanol followed by hexanes. This crude material (3.43 g) was used in the next reaction without further purification. λ(abs) 629 nm; λ(ems) 640 nm; ε 280000 1·cm⁻¹·mol⁻¹; ¹H NMR (500 MHz, CDCl₃) δ 1.75 (br. s, 12H), 3.55 (br. s, 6H), 5.92 (br. s, 2H), 6.98 (d, J=7.8 Hz, 2H), 7.13 (dd, J₁=J₂=7.4 Hz, 2H), 7.28-7.35 (m, 4H).

(221): The crude 220 (3.43 g) was dissolved in 25 ml CH₂Cl₂ to give a deep blue solution. To this solution was added methyl trifluoromethanesulfonate (4.77 g, 29 mmol, 3.2 ml, 5.8 eq.) at room temperature. After 2.5 h TLC (20/1 CHCl₃/MeOH) showed the consumption of the starting material. The mixture was quenched with saturated aqueous NaHCO₃ and extracted three times with CH₂Cl₂. The organic layer was separated, dried with anhydrous MgSO₄ and evaporated. Flash chromatography (silica gel, 200/1 to 10/1 CHCl₃/MeOH) of the residue gave 185 (2.77 g, 4.7 mmol, 94% over two steps). ¹H NMR (500 MHz, CDCl₃) 1.65 (s, 12H), 3.71 (s, 6H), 4.68 (s, 3H), 5.80 (s, 2H), 7.18 (d, J=7.9 Hz, 2H), 7.23-7.25 (m, 2H), 7.33-7.38 (m, 4H).

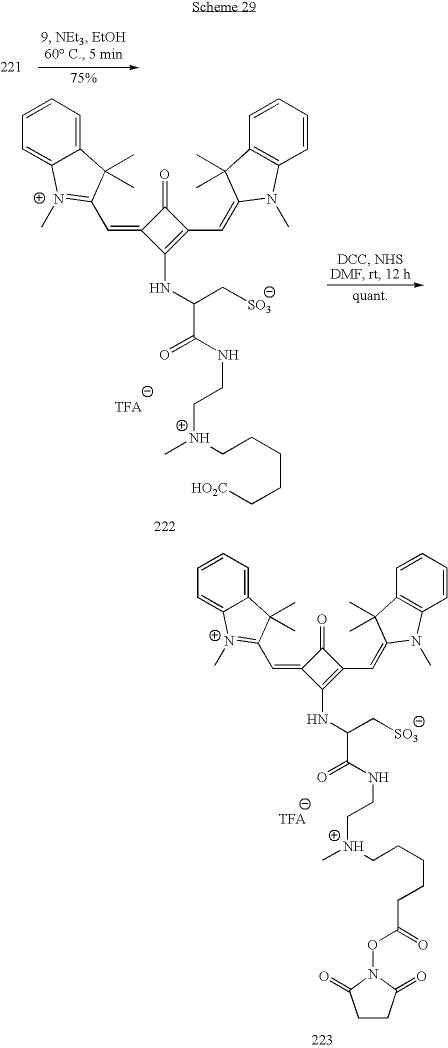

(222): The O-methylated compound 221 (59 mg, 0.1 mmol) was dissolved in 5 ml ethanol (50 ml/mmol) together with the salt 9 (68 mg, 0.15 mmol, 1.5 eq.). Triethylamine (61 mg, 0.6 mmol, 82 μl, 6 eq.) was added and the deep blue solution slightly heated in an oil bath. After 5 min TLC (10/1 CH₂Cl₂/MeOH) showed the consumption of the starting material. The solvent was removed and the dark blue residue purified by reversed phase HPLC (B 37% to 87% over 50 min, flow=20 ml/min, λ=550 nm, tᵣₑₜₑₙₜᵢₒₙ=17 min) to give 222 (64 mg, 0.075 mmol, 75%). λ(abs) 644 nm; λ(ems) 657 nm; ε 127300 1·cm⁻¹·mol⁻¹; QE 2.9%; MS [ESI] M⁺ 746.35; ¹H NMR (500 MHz, CDCl₃) δ 1.68 (s, 12H), 1.68-1.95 (m, 4H), 2.30-2.45 (m, 2H), 2.93 (s, 3H), 3.20-3.69 (m, 6H), 3.69 (s, 6H), 3.69-4.05 (m, 4H), 5.24-5.25 (m, 1H), 5.95-6.15 (m, 2H), 7.05-7.40 (m, 8H).

(223): In a flask was placed 222 (23 mg, 0.026 mmol), dicyclohexylcarbodiimide (55 mg, 0.26 mmol, 10 eq.) and N-hydroxysuccinimide (30 mg, 0.26 mmol, 10 eq.). After adding 2 ml dry DMF (77 ml/mmol) the deep blue solution was stirred overnight and afterwards evaporated to dryness. Purification of the residue by reversed phase HPLC (B 33% to 100% over 50 min, flow=4 ml/min, λ=550 nm, tᵣₑₜₑₙₜᵢₒₙ=18 min) gave 223 (25 mg, 0.026 mmol, 100%). λ(abs) 649 nm; λ(ems) 659 nm; ε 101000 1·cm⁻¹·mol⁻¹; QE 4%; MS [ESI] M⁺ 843.37; ¹H NMR (500 MHz, CDCl₃) δ 1.71 (s, 12H), 1.71-1.98 (m, 4H), 2.60-2.71 (m, 2H), 2.81 (s, 4H), 2.92 (s, 3H), 2.92-3.00 (m, 1H), 3.10-3.60 (m, 7H), 3.67 (s, 3H), 3.71 (s, 3H), 3.71-4.10 (m, 2H), 5.20-5.30 (m, 1H), 5.94 (s, 2H), 7.05-7.45 (m, 8H).

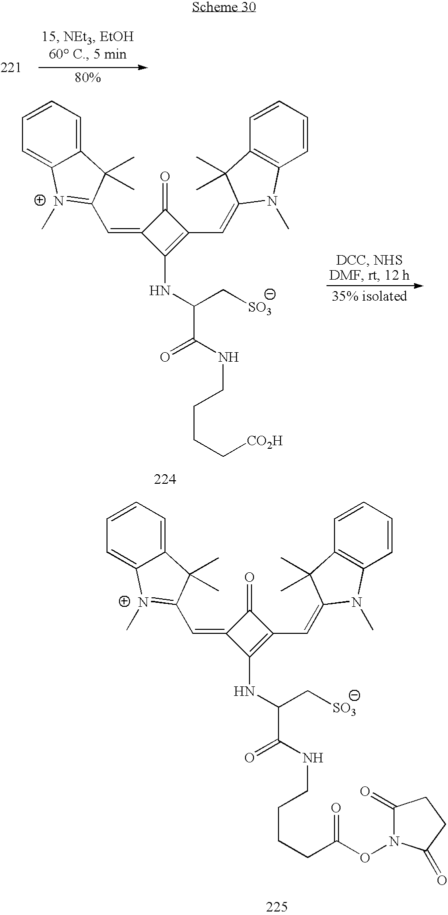

(224): The O-methylated compound 221 (54 mg, 0.09 mmol) was dissolved in 4 ml ethanol (45 ml/mmol) together with the salt 15 (31 mg, 0.11 mmol, 1.2 eq.). Triethylamine (55 mg, 0.55 mmol, 76 μl, 6 eq.) was added and the deep blue solution slightly heated in an oil bath. After 10 min TLC (10/1 CH₂Cl₂/MeOH) showed the consumption of the starting material. The solvent was removed and the dark blue residue purified by reversed phase HPLC (B 33% to 83% over 50 min, flow=20 ml/min, λ=500 nm, tᵣₑₜₑₙₜᵢₒₙ=22 min) to give 224 (49 mg, 0.073 mmol, 80%). NMR (500 MHz, CDCl₃) δ 1.60 (s, 3H), 1.66 (s, 3H), 1.71 (s, 6H), 1.60-1.75 (m, 4H), 2.30-2.40 (m, 2H), 3.10-3.20 (br. s, 1H), 3.45-3.52 (br. s, 1H), 3.52-3.90 (m, 2H), 3.59 (s, 3H), 3.73 (s, 3H), 5.20-5.30 (m, 1H), 6.26 (s, 2H), 7.05-7.45 (m, 8H).

(225): In a flask was placed 224 (29 mg, 37 umol), dicyclohexylcarbodiimide (46 mg, 222 umol, 6 eq.) and N-hydroxysuccinimide (34 mg, 296 mmol, 8 eq.). After adding 2 ml dry DMF the deep blue solution was stirred overnight and afterwards evaporated to dryness. Purification of the residue by reversed phase HPLC (B 40% to 60% over 30 min, flow=20 ml/min, λ=550 nm, tᵣₑₜₑₙₜᵢₒₙ=18 min) gave 225 (26 mg, 82%). ¹H NMR (300 MHz, CDCl₃) δ 1.50-1.80 (m, 4H), 1.66 (s, 12H), 2.50-2.60 (m, 2H), 2.70 (s, 4H), 3.25-3.40 (m, 2H), 3.50-3.80 (m, 8H), 5.28 (br. s, 1H), 5.98 (br. s, 2H), 7.00-7.45 (m, 8H).

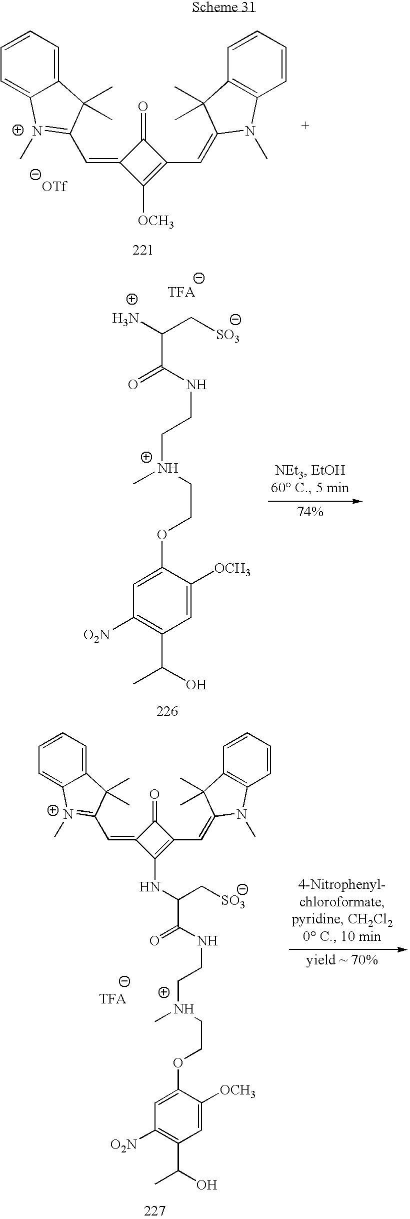

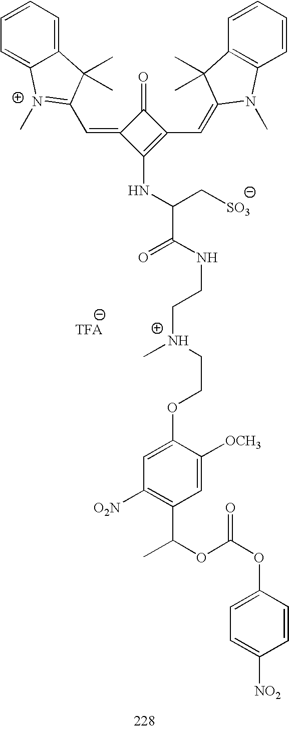

(227): The O-methylated compound 221 (32 mg, 0.054 mmol) was dissolved in 5 ml ethanol (93 ml/mmol) together with the salt 226 (31 mg, 0.054 mmol, 1 eq.). Triethyl amine (33 mg, 0.32 mmol, 44 μl, 6 eq.) was added and the deep blue solution slightly heated in an oil bath. After 10 min TLC (10/1 CH₂Cl₂/MeOH) showed the consumption of the starting material. The solvent was removed and the dark blue residue purified by reversed phase HPLC (B 33% to 83% over 50 min, flow=20 ml/min, λ=550 nm, tᵣₑₜₑₙₜᵢₒₙ=22 min) to give 227 (39 mg, 0.04 mmol, 74%). λ(abs) 644 nm; λ(ems) 657 nm; ε 101400 1·cm⁻¹·mol⁻¹; QY 2.9%; MS [ESI] M⁺ 871.36; ¹H NMR (500 MHz, CDCl₃) δ 1.50 (br. s, 3H), 1.66 (s, 3H), 1.70 (s, 6H), 1.72 (s, 3H), 3.12 (s, 3H), 3.12-3.30 (m, 2H), 3.30-3.90 (m, 6H), 3.69 (s, 3H), 3.94 (s, 3H), 4.44 (m^{AB}, 1H), 4.57 (m^{AB}, 1H), 5.22 (m, 1H), 5.51 (m, 1H), 5.90-6.15 (m, 2H), 7.00-7.15 (m, 2H), 7.18-7.25 (m, 2H), 7.30-7.40 (m, 5H), 7.61 (s, 1H).

(228): To a solution of 227 (12 mg, 0.012 mmol) in 2 ml CH₂Cl₂ (166 ml/mmol) was added pyridine (10.4 mg, 0.13 mmol, 11 μl, 11 eq.) and 4-Nitrophenyl chloroformate (24 mg, 0.12 mmol, 10 eq.) at 0° C. After 10 min reversed phase TLC (2/1 MeOH/H₂O) showed the consumption of the starting material. The solvent was evaporated and the dark blue residue purified by reversed phase HPLC (B 33% to 83% over 50 min, flow=4 ml/min, λ=500 nm, tᵣₑₜₑₙₜᵢₒₙ=18 min) to give 228 in a mixture with an unidentified impurity (13 mg, yield of 228 ˜70%). MS [ESI] M⁺ 1036.37; ¹H NMR (500 MHz, CDCl₃) δ 1.60-1.80 (m, 15H), 3.15 (s, 3H), 3.40-4.70 (m, 18H), 5.22 (m, 1H), 5.70-5.90 (m, 2H), 6.50-6.59 (m, 1H), 7.00-7.40 (m, 11H), 7.60-7.72 (s, 1H), 8.20-8.25 (m, 2H).

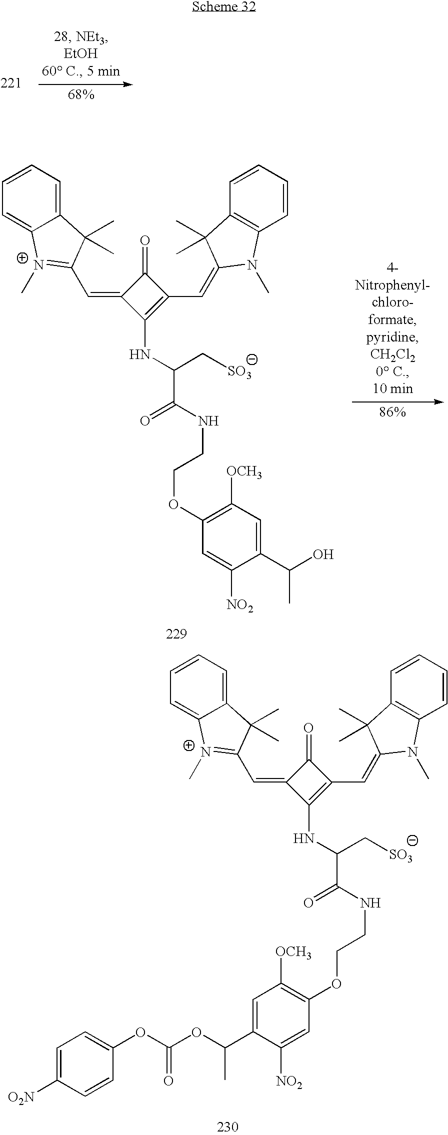

(229): The O-methylated compound 221 (80 mg, 0.14 mmol) was dissolved in 5 ml ethanol (37 ml/mmol) together with the salt 28 (61 mg, 0.15 mmol, 1.1 eq.). Triethyl amine (83 mg, 0.82 mmol, 114 μl, 6 eq.) was added and the deep blue solution slightly heated in an oil bath. After 10 min reversed phase TLC (2/1 MeOH/H₂O) showed the consumption of the starting material. The solvent was removed and the dark blue residue purified by flash chromatography through a short column of silica gel (100/1 to 5/1 CHCl₃/MeOH) to give 229 (76 mg, 0.093 mmol, 68%). MS [ESI] MH⁺ 814.31; ¹H NMR (300 MHz, CDCl₃) δ 1.40-1.80 (m, 15H), 3.00-3.30 (m, 2H), 3.40-4.00 (m, 11H), 4.15-4.40 (m, 2H), 5.03-5.23 (m, 1H), 5.40-5.50 (m, 1H), 6.32 (s, 2H), 6.80-6.90 (m, 1H), 7.08-7.40 (m, 8H), 7.63 (d, J=7.8 Hz, 1H).

(230): To a solution of 229 (73 mg, 0.09 mmol) in 3 ml CH₂Cl₂ (33 ml/mmol) was added pyridine (18 mg, 0.23 mmol, 18 μl, 2.5 eq.) and 4-Nitrophenyl chloroformate (36 mg, 0.18 mmol, 2 eq.) at 0° C. After 10 min reversed phase TLC (2/1 MeOH/H₂O) showed the consumption of the starting material. The solvent was removed and the dark blue residue purified by flash chromatography through a short column of silica gel (200/1 to 10/1 CHCl₃/MeOH) to give 230 (76 mg, 0.078 mmol, 86%). λ(abs) 646 nm; λ(ems) 661 nm; ε 79400 1·cm⁻¹·mol⁻¹; QE 3.3%; MS [ESI] MH⁺ 979.32; ¹H NMR (300 MHz, CDCl₃) δ 1.60 (br. s, 3H), 1.80 (br. s, 12H), 3.50-3.90 (m, 10H), 3.92 (s, 3H), 3.15-3.22 (br. s, 2H), 5.20-5.30 (m, 1H), 6.41 (s, 2H), 6.41-6.50 (m, 1H), 6.80-6.90 (m, 2H), 7.00-7.40 (m, 10H), 7.72 (d, J=7.6 Hz, 1H), 8.15-8.23 (m, 2H).

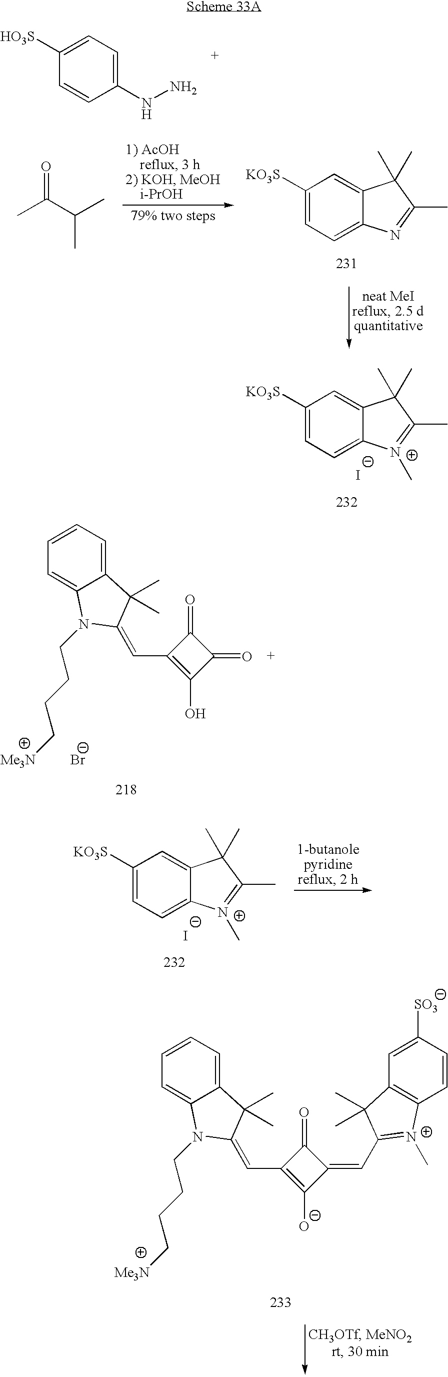

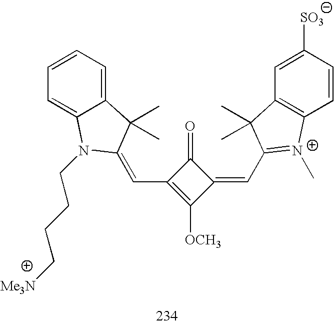

(231): 4-Hydrazinobenzene sulfonic acid hemihydrate (10 g, 53 mmol) and 3-Methyl-2-butanone (13.8 g, 160 mmol, 17 ml, 3 eq.) were dissolved in 30 ml of acetic acid (0.6 ml/mmol) followed by 3 h reflux. The mixture was suspended in diethylether and filtered. The solid was washed with diethylether to give crude indolenium sulfonate which was used without any further purification in the next reaction. The crude product was suspended in 50 ml methanol (1 ml/mmol) and 300 ml iso-propanol. To this was added potassium hydroxide (3.9 g, 68.9 mmol, 1.3 eq.) and the suspension stirred at room temperature until the color became pale yellow. The solid was filtered off and washed with iso-propanol to give 231 (11.56 g, 41.7 mmol, 79%) as a pale yellow solid. ¹H NMR (500 MHz, DMSO) δ 1.21 (s, 6H), 2.18 (s, 3H), 7.30 (d, J=7.3 Hz, 1H), 7.49 (d, J=7.3 Hz, 1H), 7.58 (s, 1H).

(232): In a sealed tube 231 (2.77 g, 10 mmol) was refluxed in 10 ml methyliodide (1 ml/mmol) for 2.5 days. The resulting solid was filtered and washed with a 10/1 hexanes/ethyl acetate mixture to give 232 (4.88 g, quantitative). MS [ESI] MH⁺ 254.08, MK⁺ 292.04; ¹H NMR (300 MHz, D₂O) δ 1.49 (s, 6H), 3.93 (s, 3H), 7.73 (d, J=7.6 Hz, 1H), 7.91 (d, J=7.3 Hz, 1H), 7.99 (s, 1H), no signal for 2-methyl due to exchange.

(233): To a solution of the semisquaric acid 218 (310 mg, 0.84 mmol) in 13 ml 1-butanol (15 ml/mmol) and 2.6 ml pyridine (3 ml/mmol) was added 232 (530 mg, 1.26 mmol, 1.5 eq.). The mixture was refluxed for 24 h and then cooled to 0° C. The precipitate was filtered off and washed with 1-butanol followed by diethyl ether to give crude 233 (612 mg) which was used in the next reaction without further purification. A sample was purified by reversed phase HPLC. λ(abs) 633 nm; λ(ems) 646 nm; ε 57000 1·cm⁻¹·mol⁻¹; QE 75.5%; MS [ESI] MH⁺ 604.28; ¹H NMR (300 MHz, MeOD) δ 1.72 (s, 6H), 1.73 (s, 6H), 1.80-1.90 (m, 2H), 1.95-2.06 (m, 2H), 3.13 (s, 9H), 3.39-3.47 (m, 2H), 3.61 (s, 3H), 4.13-4.21 (m, 2H), 5.91 (s, 1H), 5.94 (s, 1H), 7.19-7.50 (m, 5H), 7.80-7.86 (m, 2H).

(234): The crude 233 (100 mg, 0.17 mmol) was dissolved in 3 ml nitromethane (18 ml/mmol) to give a deep blue solution. To this solution was added methyl trifluoromethansulfonate (272 mg, 0.17 mmol, 0.18 ml, 10 eq.) at room temperature. After 30 min reversed phase TLC (TCL C₁₈ 2/1 MeOH/H₂O) showed the consumption of the starting material. The mixture was quenched with water and extracted three times with CHCl₃. The organic layer was separated, dried with anhydrous MgSO₄ and evaporated. The residue was used in the next step without further purification.

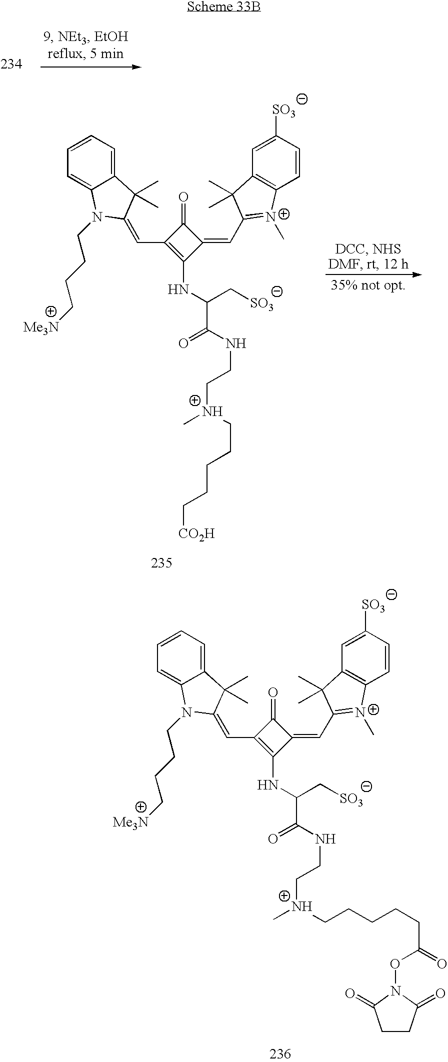

(235): The crude O-methylated compound 234 (91 mg, 0.12 mmol) was dissolved in 5 ml ethanol (42 ml/mmol) together with the salt 9 (59 mg, 0.13 mmol, 1.1 eq.). Triethyl amine (0.5 ml, 4 ml/mmol) was added and the deep blue solution slightly heated in an oil bath. After 5 min reversed phase TLC (2/1 MeOH/H₂O) showed the consumption of the starting material. The solvent was removed and the dark blue residue purified by reversed phase HPLC (B 0% to 60% over 50 min, flow=20 ml/min, λ=500 nm, tᵣₑₜₑₙₜᵢₒₙ=31 min) to give 235 (31 mg, 0.03 mmol, 25% yield from crude 234). ¹H NMR (500 MHz, D₂O) δ 1.15-1.23 (m, 2H), 1.40 (br. s, 6H), 1.47 (br. s, 6H), 1.47-1.60 (m, 2H), 1.65-1.75 (m, 2H), 1.75-1.85 (m, 2H), 2.12-2.21 (m, 2H), 2.70-3.60 (series of m, 16H), 2.95 (s, 9H), 4.03-4.11 (m, 2H), 4.60-4.80 (m, 2H), 5.10 (br. s, 1H), 5.40-5.70 (br. m, 2H), 7.05-7.40 (m, 5H), 7.65-7.70 (m, 2H).

(236): In a flask was placed 235 (20 mg, 0.019 mmol), dicyclohexylcarbodiimide (20 mg, 0.096 mmol, 5 eq.) and N-hydroxysuccinimide (11 mg, 0.096 mmol, 5 eq.). After adding 3 ml dry DMF (160 ml/mmol) the deep blue solution was stirred overnight and afterwards evaporated to dryness. Purification of the residue by reversed phase HPLC (B 0% to 60% over 50 min, flow=20 ml/min, λ=500 nm, tᵣₑₜₑₙₜᵢₒₙ=33 min) gave 236 (7.5 mg, 0.007 mmol, 35% not optimized). λ(abs) 652 nm; λ(ems) 665 nm; ε 39800 1·cm⁻¹·mol⁻¹; QE 28%; MS [ESI] M⁺ 1022.43.

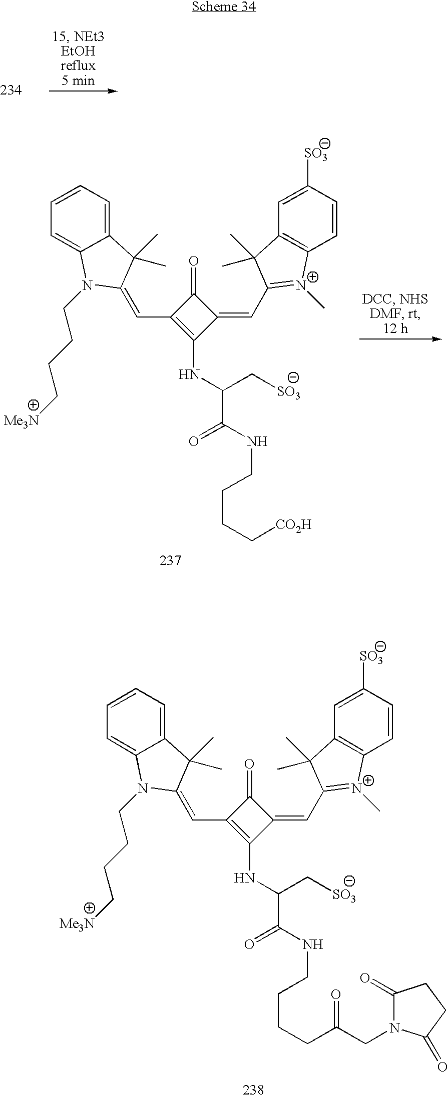

(237): The crude O-methylated compound 234 (180 mg, 0.23 mmol) was dissolved in 5 ml ethanol (22 ml/mmol) together with the salt 15 (80 mg, 0.28 mmol, 1.2 eq.). Triethyl amine (0.5 ml, 2.2 ml/mmol) was added and the deep blue solution slightly heated in an oil bath. After 5 min reversed phase TLC (2/1 MeOH/H₂O) showed the consumption of the starting material. The solvent was removed and the dark blue residue purified by reversed phase HPLC (B 10% to 50% over 50 min, flow=20 ml/min, λ=500 nm, tᵣₑₜₑₙₜᵢₒₙ=29 min) to give 237 (69 mg, 0.08 mmol, 34% yield from crude 160). ¹H NMR (300 MHz, MeOD) δ 1.50-1.60 (m, 4H), 1.70 (br. s, 12H), 1.70-1.75 (m, 2H), 2.00-2.15 (m, 2H), 2.20-2.40 (m, 2H), 3.12 (s, 9H), 3.15-3.35 (m, 2H), 3.40-3.52 (m, 4H), 3.60-3.70 (m, 3H), 4.12-4.32 (m, 2H), 5.10-5.20 (m, 2H), 5.80-6.10 (br. m, 2H), 7.20-7.51 (m, 5H), 7.80-7.91 (m, 2H).

(238): In a flask was placed 237 (62 mg, 0.071 mmol), dicyclohexylcarbodiimide (147 mg, 0.71 mmol, 10 eq.) and N-hydroxysuccinimide (82 mg, 0.71 mmol, 10 eq.). After adding 3 ml dry DMF (160 ml/mmol) the deep blue solution was stirred overnight and afterwards evaporated to dryness. Purification of the residue by reversed phase HPLC (B 10% to 50% over 50 min, flow=20 ml/min, λ=500 nm, tᵣₑₜₑₙₜᵢₒₙ=33 min) gave 238 (14 mg, 0.014 mmol, 20% not optimized) alongside recovered starting material. ¹H NMR (500 MHz, MeOD+CD₃CN) δ 1.53-1.63 (m, 2H), 1.70 (br. s, 12H), 1.70-1.75 (m, 2H), 1.90-2.10 (m, 2H), 2.53-2.60 (m, 2H), 2.65 (s, 4H), 3.08 (s, 9H), 3.15-3.35 (m, 2H), 3.40-3.52 (m, 4H), 3.60-3.70 (m, 3H), 4.12-4.20 (m, 2H), 5.00-5.10 (m, 2H), 5.65-5.92 (br. m, 2H), 7.20-7.32 (m, 3H), 7.35-7.41 (m, 1H), 7.46-7.52 (m, 2H), 7.80-7.90 (m, 2H).

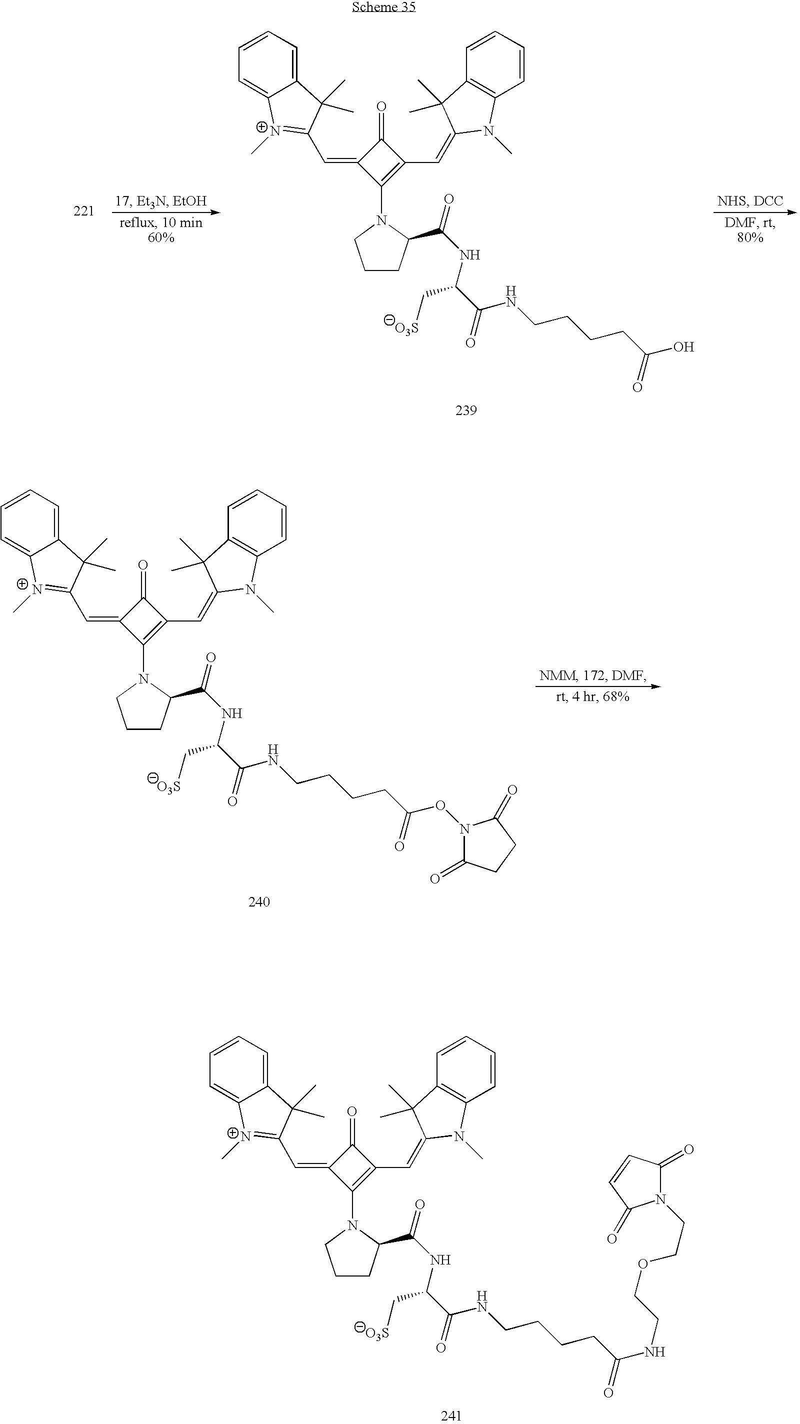

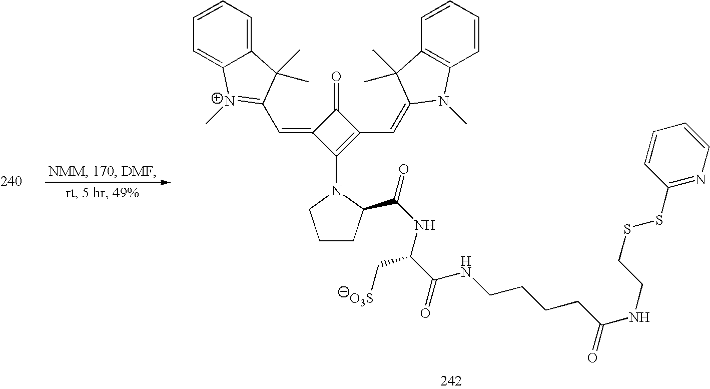

2-[2-(5-((R)-2-(R)-pyrrolidine-2-carboxamido)-3-sulfonopropionamido)-pentanoic acid)-4-oxo-3-(1,3,3-trimethyl-2,3-dihydro-1H-2-indolylidenmethyl)-2-cyclobutenyl-idenmethyl]-1,3,3-trimethyl-3H-indolium (239). 242-Methoxy-4-oxo-3-(1,3,3-trimethyl-2,3-dihydro-1H-2-indolylidenmethyl)-2-cyclobutenylidenmethyl]-1,3,3-trimethyl-3H-indolium trifluoroacetate (221) (114 mg, 0.194 mmol) and acid 17 (71 mg, 0.194 mmol) to were placed under argon in a 25 mL rbf and dissolved in EtOH (10 mL). Triethylamine (0.14 mL, 1.00 mmol) was added, and the mixture was stirred at reflux for 20 min before the solvent was removed in vacuo. The crude product was purified via reverse phase HPLC using a gradient of 2:3 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 500 nm. The product was collected at 14 min. The solution containing the product was frozen, and the solvents removed via the use of a lyophilizer providing 239 as a blue solid (90 mg, 0.116 mmol, 60%): R_{f}: 0.30 (3:1 dichloromethane-methanol); ¹H NMR (500 MHz, CD₃OD) δ 1.45 (m, 2H), 1.52 (p, J=7.5 Hz, 2H), 1.65-1.70 (m, 14H), 2.16 (m, 1H), 2.19-2.25 (m, 1H), 2.22 (t, J=7.5 Hz, 2H), 2.42 (m, 2H), 2.98 (m, 1H), 3.05-3.16 (m, 3H), 3.70 (s, 3H), 3.74 (s, 3H), 4.21 (q, J=8.5 Hz, 1H), 4.44 (m, 1H), 4.65 (dd, J=8.5, 5 Hz, 1H), 5.18 (t, J=5 Hz, 1H), 7.26 (t, J=7.5 Hz, 2H), 7.31 (m, 2H), 7.41 (t, J=7.5 Hz, 2H), 7.46 (m, 2H); ¹³C NMR (125 MHz, CD₃OD) δ 23.34, 23.43, 24.80, 26.68, 26.85, 27.07, 29.74, 29.79, 32.46, 33.82, 34.38, 34.55, 40.19, 40.26, 51.25, 51.43, 52.80, 52.86, 53.05, 65.88, 88.57, 112.14, 112.39, 112.68, 123.42, 126.37, 126.58, 129.61, 142.79, 143.32, 144.55, 144.78, 162.18, 162.38, 168.01, 171.86, 173.34, 175.93, 176.43, 177.21, 177.28; HRMS [M+Na] calcd for C₄₁H₄₉NaN₅O₈S⁺ 794.3194. found 794.3197.

2-[2-(5-((R)-2-(R)-pyrrolidine-2-carboxamido)-3-sulfono-propionamido)-pentanoic acid)-4-oxo-3-(1,3,3-trimethyl-2,3-dihydro-1H-2-indolylidenmethyl)-2-cyclobutenyl-idenmethyl]-1,3,3-trimethyl-3H-indolium, succinimidyl ester (240): Acid 239 (90 mg, 0.116 mmol), NHS (133 mg, 1.16 mmol) and DCC (200 mg, 0.969 mmol) were placed under argon in a 25 mL rbf. Anhydrous DMF (6 mL) was added, and the mixture was stirred at ambient temperature for 15 h. The DMF was removed via the use of a lyophilizer, and the crude product was purified via reverse phase HPLC using a gradient of 2:3 ([95% CH₃CN/4.9% H₂O/0.1% TFA]:[99.9% H₂O/0.1% TFA]) to 3:2 ([95% CH₃CN/4.9% H₂O/0.1% TFA]:[99.9% H₂O/0.1% TFA]) over 30 min at a flow rate of 20 mL/min, monitoring at 500 nm. The product was collected at 19 min. The solution containing the product was frozen, and the solvents removed via the use of a lyophilizer providing 240 as a blue solid (81 mg, 0.0932 mmol, 80%): R_{f}: 0.51 (9:1 dichloromethane-methanol); ¹H NMR (500 MHz, DMSO) δ 1.35 (p, J=7.5 Hz, 2H), 1.49 (p, J=7.5 Hz, 2H), 1.61 (s, 3H), 1.63 (s, 3H), 1.64 (s, 3H), 1.65 (s, 3H), 2.03 (m, 2H), 2.27 (m, 2H), 2.54 (t, J=7.5 Hz, 2H), 2.71 (dd, J=13.5, 8.5 Hz, 1H), 2.76-2.84 (m, 2H), 2.79 (s, 4H), 2.95 (m, 1H), 3.67 (s, 3H), 3.68 (s, 3H), 4.19 (q, J=8.5 Hz, 1H), 4.36 (m, 1H), 4.42 (m, 1H), 5.26 (t, J=5 Hz, 1H), 5.69 (s, 1H), 5.99 (s, 1H), 7.21-7.27 (m, 2H), 7.40-7.41 (m, 3H), 7.46 (d, J=8 Hz, 1H), 7.54 (d, J=7.5 Hz, 2H), 8.02 (t, J=4.5 Hz, 1H) (NH), 8.52 (d, J=6 Hz, 1H) (NH); ¹³C NMR (125 MHz, DMSO) δ 21.44, 23.10, 25.41, 25.49, 25.57, 26.00, 26.07, 27.88, 29.66, 30.77, 32.30, 32.57, 37.80, 49.10, 49.52, 51.10, 51.40, 52.19, 63.72, 87.72, 88.59; HRMS [M+Na] calcd for C₄₅H₅₂NaN₆O₁₀S⁺ 891.3358. found 891.3346.

2-[2-(5-(N-maleimido-3-oxapentyl((R)-2-(R)-pyrrolidine-2-carboxamido)-3-sulfono-propionamido)-pentanamide))-4-oxo-3-(1,3,3-trimethyl-2,3-dihydro-1H-2-indolyl-idenmethyl)-2-cyclobutenylidenmethyl]-1,3,3-trimethyl-3H-indolium (241): Succinimidyl ester 240 (14.0 mg, 0.0161 mmol) and N-(5-amino-3-oxapentyl) maleimide trifluoroacetate 172 (7.0 mg, 0.0235 mmol) were placed under argon in a 10 mL rbf. Anhydrous DMF (2 mL) was added to the flask, followed by distilled NMM (17.0 μL, 0.158 mmol). The mixture was stirred at ambient temperature for 4 h. The solvent was removed via the use of a lyophilizer, and the crude product was purified via reverse phase HPLC using a gradient of 3:7 ([95% CH₃CN/4.9% H₂O/0.1% TFA]:[99.9% H₂O/0.1% TFA]) to 55:45 ([95% CH₃CN/4.9% H₂O/0.1% TFA]:[99.9% H₂O/0.1% TFA]) over 30 min at a flow rate of 20 mL/min, monitoring at 500 nm. The product was collected at 26 min. The solution containing the product was frozen, and the solvents removed via the use of a lyophilizer providing 241 as a blue solid (10.3 mg, 0.0110 mmol, 68%): R_{f}: 0.39 (9:1 dichloromethane-methanol); ¹H NMR (500 MHz, d-DMSO) δ 1.24 (p, J=7.5 Hz, 2H), 1.35 (p, J=7.5 Hz, 2H), 1.61 (s, 3H), 1.62 (s, 3H), 1.64 (s, 3H), 1.65 (s, 3H), 1.95 (t, J=7.5 Hz, 2H), 2.03 (m, 2H), 2.27 (m, 2H), 2.70 (dd, J=13.5, 8 Hz, 1H), 2.76-2.82 (m, 2H), 2.92 (dt, J=11.5, 5.5 Hz, 2H), 3.34 (t, J=6 Hz, 2H), 3.48 (t, J=6 Hz, 2H), 3.54 (t, J=5.5 Hz, 2H), 3.67 (s, 6H), 4.19 (q, J=8.5 Hz, 1H), 4.36-4.43 (m, 2H), 5.24 (t, J=5.5 Hz, 2H), 5.68 (s, 1H), 5.98 (s, 1H), 7.01 (s, 2H), 7.22-7.27 (m, 2H), 7.38-7.41 (m, 3H), 7.46 (d, J=8 Hz, 1H), 7.54 (d, J=7 Hz, 2H), 7.70 (t, J=5.5 Hz, 1H) (NH), 7.96 (t, J=5.5 Hz, 1H) (NH), 8.52 (d, J=6 Hz, 1H) (NH); ¹³C NMR (125 MHz, d-DMSO) δ 22.54, 23.06, 25.52, 25.97, 26.06, 28.45, 30.74, 32.30, 32.55, 34.82, 36.69, 38.25, 38.30, 49.10, 49.50, 51.03, 51.37, 52.23, 63.72, 66.73, 68.58, 87.70, 88.51, 110.96, 111.48, 122.15, 124.47, 125.01, 128.11, 134.50, 141.18, 141.75, 142.56, 142.79, 159.21, 159.98, 166.05, 169.80, 169.95, 170.87, 171.94, 172.27, 173.86, 174.28; HRMS [M+Na] calcd for C₄₉H₅₉NaN₇O₁₀S⁺ 960.3936. found 960.3877.

2-[2-(5-((2-(2-(pyridin-2-yl)disulfanyl)ethanamine)-((R)-2-((R)-pyrrolidine-2-carboxamido)-3-sulfono-propionamido)-pentanamide))-4-oxo-3-(1,3,3-trimethyl-2,3-dihydro-1H-2-indolylidenmethyl)-2-cyclobutenylidenmethyl]-1,3,3-trimethyl-3H-indolium (242): Succinimidyl ester 240 (16.0 mg, 0.0184 mmol) and S-(2-pyridylthio)cysteamine hydrochloride (170) (9.0 mg, 0.0404 mmol) were placed under argon in a 5 mL rbf. Anhydrous DMF (1 mL) was added, and the mixture was stirred at ambient temperature for 5 h. The solvent was removed via the use of a lyophilizer, and the crude product was purified via reverse phase HPLC using a gradient of 3:7 ([95% CH₃CN/4.9% H₂O/0.1% TFA]: [99.9% H₂O/0.1% TFA]) to 65:35 ([95% CH₃CN/4.9% H₂O/0.1% TFA]:[99.9% H₂O/0.1% TFA]) over 30 min at a flow rate of 20 mL/min, monitoring at 500 nm. The product was collected at 23 min. The solution containing the product was frozen, and the solvents removed via the use of a lyophilizer providing 242 as a blue solid (8.4 mg, 8.93 μmol, 48.5%): R_{f}: 0.38 (9:1 dichloromethane-methanol); ¹H NMR (500 MHz, d-DMSO) δ 1.24 (p, J=7.5 Hz, 2H), 1.36 (p, J=7.5 Hz, 2H), 1.61 (s, 3H), 1.62 (s, 3H), 1.64 (s, 3H), 1.64 (s, 3H), 1.96 (t, J=7.5 Hz, 2H), 2.03 (m, 2H), 2.27 (m, 2H), 2.70 (dd, J=13.5, 8 Hz, 1H), 2.76-2.83 (m, 2H), 2.87 (t, J=6.5 Hz, 2H), 2.92 (p, J=6.5 Hz, 1H), 3.29 (q, J=6.5 Hz, 2H), 3.67 (s, 6H), 4.18 (q, J=8.5 Hz, 1H), 4.36 (m, 1H), 4.42 (q, J=6.5 Hz, 1H), 5.24 (t, J=5 Hz, 1H), 5.67 (s, 1H), 5.98 (s, 1H), 7.22-7.26 (m, 3H), 7.38-7.40 (m, 3H), 7.45 (d, J=8 Hz, 1H), 7.54 (d, J=7 Hz, 2H), 7.76 (d, J=8 Hz, 1H), 7.82 (t, J=8 Hz, 1H), 7.97 (m, 2H), 8.44 (d, J=4.5 Hz, 1H) (NH), 8.52 (d, J=6 Hz, 1H) (NH); ¹³C NMR (125 MHz, d-DMSO) δ 22.48, 23.07, 25.53, 25.97, 26.06, 28.40, 30.75, 32.31, 32.56, 34.89, 37.45, 37.74, 38.24, 49.11, 49.50, 51.02, 51.37, 52.25, 63.72, 87.72, 88.49, 110.98, 111.47, 119.25, 121.15, 122.15, 124.49, 125.00, 128.12, 137.80, 141.18, 141.74, 142.57, 142.79, 149.52, 159.09, 159.24, 159.97, 166.05, 169.80, 169.97, 172.06, 172.30, 173.85, 174.28; HRMS [M+H] calcd for C₄₈H₅₈N₇O₇S₃⁺ 940.3554. found 940.3499.

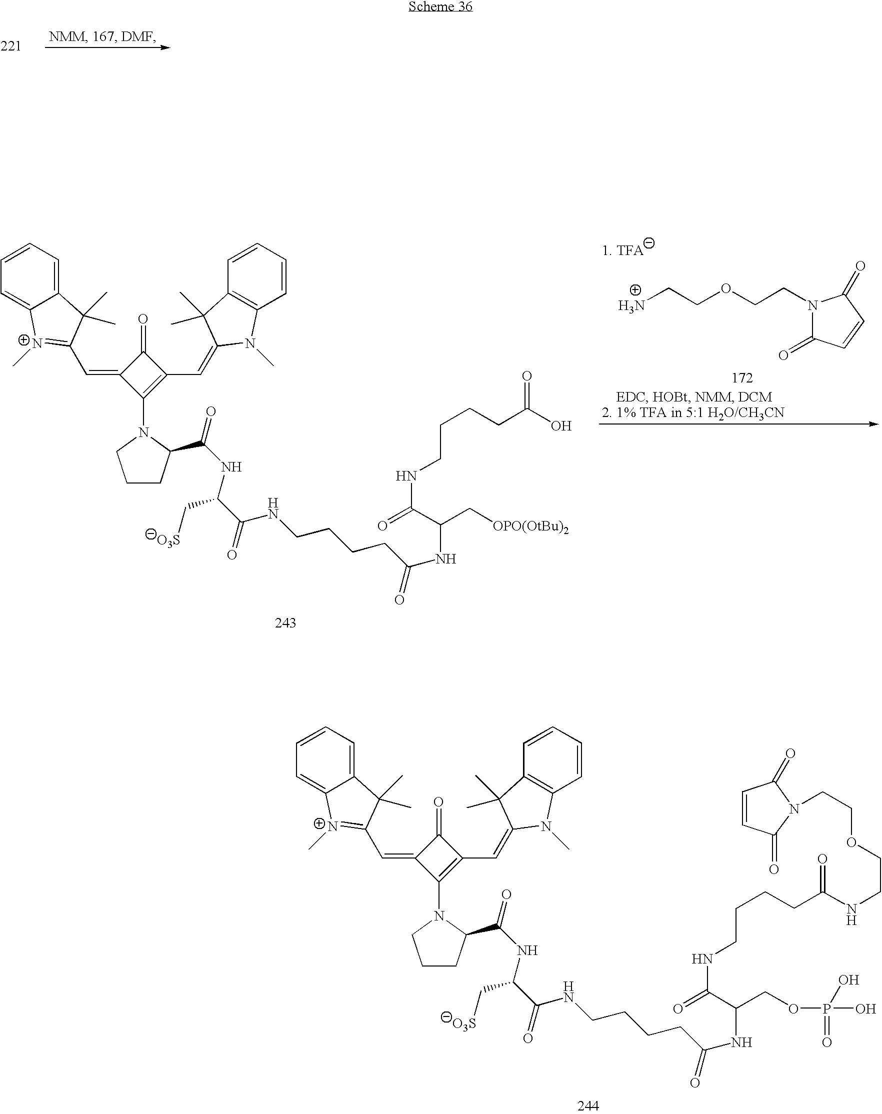

(243): To an argon flushed flask containing 221 (22 mg, 25.3 umol), 167 (30 mg, 76 umol, 3 eq), and anhydrous DMF (1 mL) was added NMM (15 uL, 152 umol, 6 eq) and the resulting solution was stirred under argon for 23 hours. The solvent was removed by vacuum and the resulting blue solid was purified by RP HPLC (B 40%-60% over 30 min, flow=20 mL/min, λ=550 nm, tᵣₑₜₑₙₜᵢₒₙ=17.8 min) to provide 14 mg of pure product (48%). ¹H NMR (300 MHz, CD₃OD) δ 8.64 (m, 1H), 7.29 (m, 8H), 5.16 (m, 1H), 4.61-4.55 (m, 2H), 4.42 (m, 1H), 3.71 (s, 3H), 3.67 (s, 3H), 3.17-2.96 (m, 6H), 2.38 (m, 2H), 2.30-2.10 (m, 6H), 1.68 (m, 12H), 1.62 (m, 8H), 1.41 (m, 18H).

(244): To an argon flushed flask containing 243 (9.2 mg, 8 umol), 172 (9.5 mg, 32 umol, 4 eq), and HOBt (4.3 mg, 32 umol, 4 eq) at 0° C. was added NMM (7 uL, 64 umol, 8 eq) followed by the addition of EDC (6 mg, 32 umol, 4 eq). The solution was stirred under argon for 19 hours at which time the solvent was removed by vacuum. The resulting blue solid was purified by RP HPLC (B 40%-60% over 30 min, flow=20 mL/min, 550 nm, tᵣₑₜₑₙₜᵢₒₙ=9.4 min) to provide 4.8 mg (50%). ¹H NMR (500 MHz, CD₃OD) δ 8.64 (m, 1H), 7.41-7.19 (m, 8H), 5.14 (m, 1H), 4.60 (t, 1H, J=6.6 Hz), 4.51 (t, 1H, J=5 Hz), 4.40 (m, 1H), 4.19-4.09 (m, 3H), 3.69-3.60 (m, 8H), 3.52 (t, 2H, J=5.5 Hz), 3.43 (t, 2H, J=5.4 Hz), 3.15-3.05 (m, 7H), 3.07 (m, 1H), 2.17-2.13 (m, 6H), 2.36 (m, 2H), 1.61-1.41 (m, 22H).

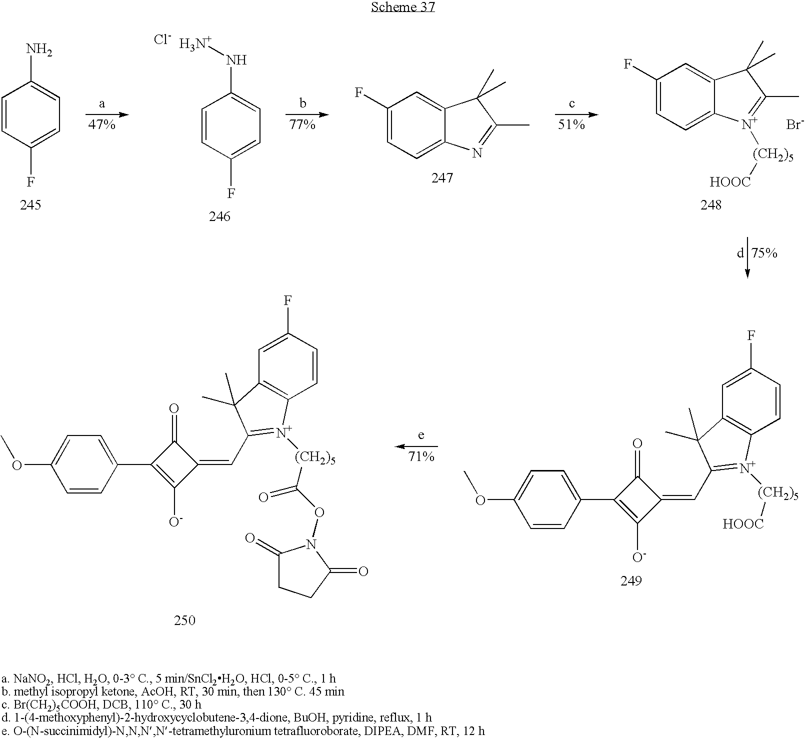

(246): 4-fluorophenylaniline (11.11 g, 0.100 mol) 245 was dissolved in 62 mL conc. HCl. The mixture was stirred in an ice bath for several minutes before adding sodium nitrite (7.94 g, 0.115 mol). After stirring for five additional minutes, SnCl₂.2H₂O (54.15 g, 0.240 mol) dissolved in 60 mL concentrated HCl was slowly added. The reaction temperature was monitored to ensure the reaction temperature did not exceed 5° C. The reaction was stirred for 1 h, then filtered to collect the tan precipitate. The precipitate was recrystallized from hot water and the crystals were lyophilized overnight. (5.87 g, 46% yield); H NMR (DMSO, 500 MHz): δ 10.81 (3H, s), 8.22 (1H, s), 7.15 (2H, t, J=9 Hz), 7.02 (2H, dd, J_{AB}=4.5 Hz, J_{AX}=9 Hz)

(247): In a flask equipped with a stir bar and condenser was added 4-fluorophenylhydrazine (3.73 g, 23.0 mmol) 246, 3-methyl-2-butanone (2.76 g, 32.1 mmol), and 23 mL acetic acid. The flask was flushed with argon and stirred at room temperature for 30 min, then placed in an oil bath at 130° C. for 45 min. The reaction mixture was then poured into 75 g crushed ice, extracted 2×75 mL with ethyl acetate, washed 2×75 mL with water, dried over Na2SO4, and the solvent was removed in vacuo. The product, a reddish oil, was then dried under vacuum overnight. (3.71 g, 77% yield). H NMR (CDCl3, 500 MHz): δ 7.44-7.47 (1H, m), 6.97-7.01 (2H, m), 2.27 (3H, s), 1.31 (6H, s).

(248): 5-fluoro-2,3,3-trimethyl-3H-indole (3.10 g, 17.5 mmol) 247 and 6-bromohexanoic acid (5.12 g, 26.2 mmol) were added to 45 mL 1,2-dichlorobenzene. The reaction vessel was fitted with a condenser, flushed with argon, and stirred in an oil bath at 110° C. for 36 hr. The solvent was lyophilized and the resulting sticky, dark crude mixture was triturated with ether to yield a dark red solid. (3.14 g, 51% yield) H NMR (DMSO-d6, 500 MHz): δ 8.05 (1H, dd, J_{AB}=4 Hz, J_{AX}=9 Hz), 7.86 (1H, dd, J_{AB}=2.5 Hz, J_{AX}=8 Hz), 7.49 (1H, td, J_{AB}=2.5 Hz, J_{AX}=8.5), 4.44 (2H, t, J=7.5 Hz), 2.83 (3H, s), 2.23 (2H, t, J=7.5 Hz), 1.83 (2H, quintet, J=7.5 Hz), 1.49-1.58 (8H, m), 1.42 (2H, quintet, J=8 Hz); ¹³C NMR (125 MHz, DMSO-d₆) δ 192.01, 182.64, 179.11, 178.05, 174.23, 166.05, 162.49, 161.92, 160.55, 145.52 (d, J=9 Hz), 137.28, 128.12, 124.50, 115.38 (d, J=24 Hz), 115.07 (d, J=8.8 Hz), 144.68, 110.71 (d, J=25 Hz), 91.12, 55.36, 51.49, 45.01, 33.36, 27.11, 25.58, 24.79, 24.07, 22.29; ESI-TOF-MS (m/z): [MH⁺] calcd for C₂₈H₂₉FNO₅ 478.2024. found 478.2016

(249): 1-(6-carboxyhexyl)-5-fluoro-2,3,3,-trimethylindolenine (0.335 g, 9.46 mmol) 248 and 1-(4-methoxyphenyl)-2-hydroxy-3,4-dione (0.193 g, 9.46 mmol) in 9 mL butanol and 0.9 mL pyridine was refluxed for 45 min. and monitored BY TLC (9:1 CH2Cl2:MeOH). The reaction vessel was cooled and the solvent removed by vacuum distillation. Separation by silica gel column chromatography (5% MeOH, 95% CH₂Cl₂) provided the product as a dark violet solid. (0.337 g, 75%) H NMR (DMSO-d₆, 500 MHz): δ 8.07 (2H, d, J=6.5 Hz), 7.78 (1H, dd, J_{AB}=4 Hz, J_{AX}=8.5 Hz), 7.75 (1H, dd, J_{AB}=3 Hz, J_{AX}=8.5 Hz), 7.38 (1H, td, J_{AB}=2 Hz, J_{AX}=9 Hz), 7.07 (2H, d, J=9 Hz), 6.23 (1H, s), 4.41 (2H, t, J=7 Hz), 3.83 (3H, s), 2.20 (2H, t, J=7 Hz), 1.72-1.83 (8H, m), 1.55 (2H, quintet, J=7.5 Hz), 1.40 (2H, quintet, J=7.5 Hz)

(250): A flask equipped with a stir bar was flushed with argon before adding 6 mL of anhydrous DMF. Compound 249 (0.318 g, 0.666 mmol) and N,N,N′,N′-Tetramethyl-O—(N-succinimidyl)uronium tetrafluoroborate (0.601 g, 2.00 mmol) were added to the DMF under argon, and then DIPEA (0.517 g, 4.00 mmol) was injected via syringe. The reaction was monitored by TLC (10% MeOH, 90% CH₂Cl₂) and stopped after 6 hrs. The DMF was lyophilized and the crude mixture separated by column chromatography on Davisil (2% MeOH, 98% CH₂Cl₂) to yield the dark violet product. (0.272 g, 71% yield) H NMR (CDCl₃, 500 MHz): δ 8.29 (2H, d, J=9 Hz), 7.19-7.23 (2H, m), 7.14 (1H, td, J_{AB}=2.5 Hz, J_{AX}=8.5 Hz), 6.97 (2H, d, J=9 Hz), 6.25 (1H, s), 4.26 (2H, t, J=7.5 Hz), 3.88 (3H, s), 2.87 (4H, s), 2.65 (2H, t, J=7 Hz), 1.83-1.94 (10H, m), 1.64 (2H, quintet, J=7.5 Hz)

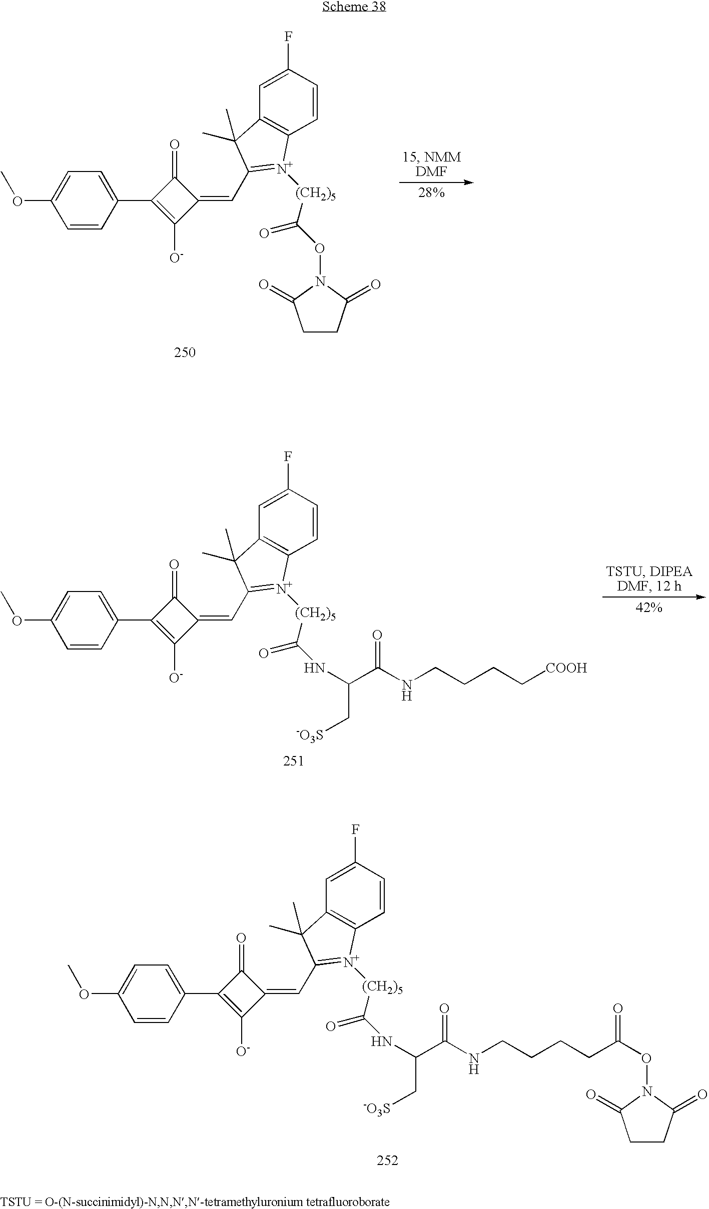

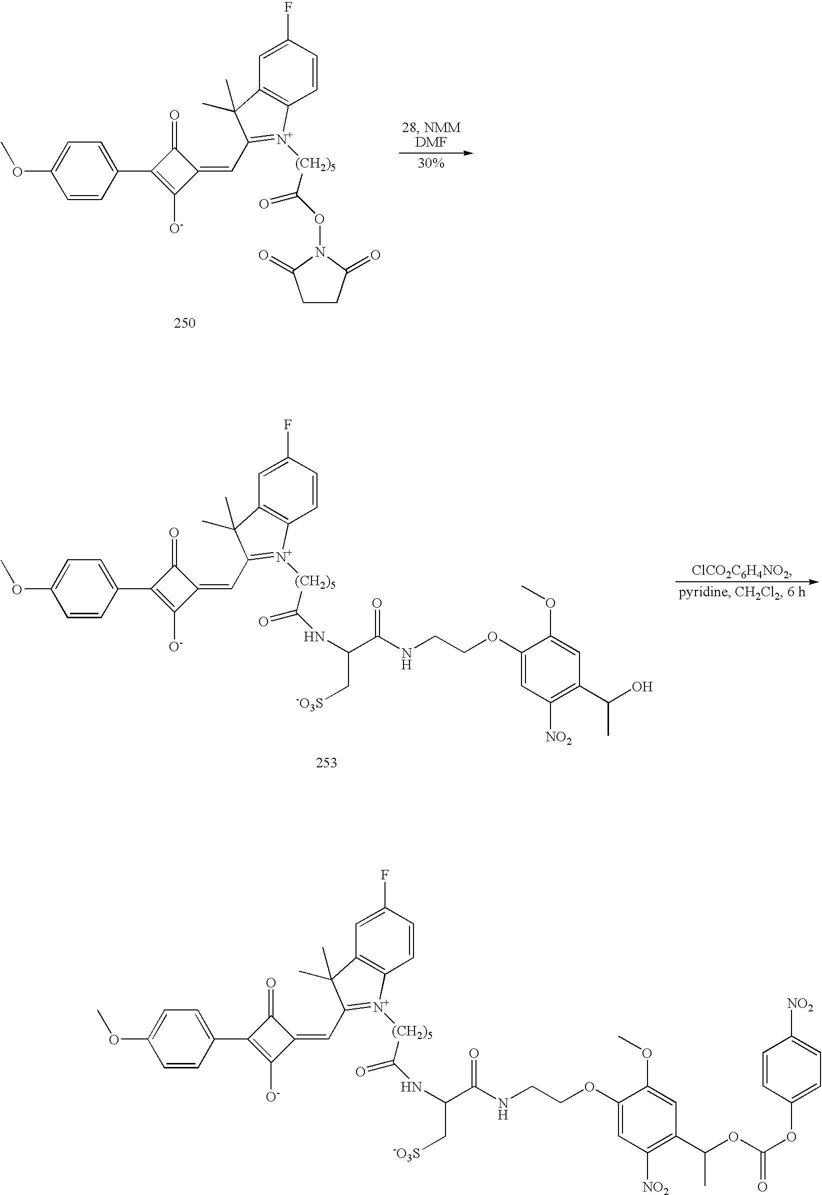

(251): A flask equipped with stir bar containing 250 (42 mg, 0.072 mmol) and the non-titratable cysteic acid sidechain 15 (84 mg, 0.29 mmol) was flushed with argon before adding DMF (0.7 μL) and NMM, (95 μL, 88 mg, 0.87 mmol). The reaction was stirred at room temperature overnight. The solvent was removed on the lyophilizer and the crude product separated by HPLC. (14.3 mg, 28%) H NMR (DMSO-d₆, 500 MHz): δ 8.07 (2H, d, J=8.5 Hz), 7.91 (1H, d, J=6 Hz), 7.87 (1H, dd, J_{AB}=4.5 Hz, J_{AX}=9 Hz), 7.78 (1H, t, J=5.5 Hz), 7.73 (1H, dd, J_{AB}=2.5 Hz, J_{AX}=8 Hz), 7.36 (1H, td, J_{AB}=2.5 Hz, J_{AX}=9 Hz), 7.07 (2H, d, J=9 Hz), 6.23 (1H, s), 4.39 (2H, t, J=7 Hz), 4.32 (1H, q, J=6.5 Hz), 3.83 (3H, s), 3.00 (2H, m), 2.81 (1H, dd, J_{AB}=4.5 Hz, J_{AX}=13.5 Hz), 2.72 (1H, dd, J_{AB}=8 Hz, J_{AX}=14 Hz), 2.1-2.25 (4H, m), 1.78 (7H, s), 1.57 (2H, m), 1.32-1.49 (7H, m); ¹³C NMR (125 MHz, DMSO-d₆) δ 191.61, 182.66, 179.11, 177.96, 174.35, 171.65, 170.59, 165.66, 162.51, 160.90, 160.56, 145.51 (d, J=8.8 Hz), 137.32, 128.14, 124.48, 115.50 (d, J=22.5 Hz), 115.34, 114.69, 110.63 (d, J=25 Hz), 91.15, 55.57, 54.87, 52.04, 50.97, 45.14, 35.00, 33.28, 28.45, 27.01, 25.43, 24.77, 24.48, 21.77; ESI-TOF-MS (m/z): [M]⁻ calcd for [C₃₆H₄₁FN₃O₁₀S]⁻ 726.2491. found 726.2485

(252): A flask containing 251 (15 mg, 0.019 mmol) and N,N,N′,N′-tetramethyl-O—(N-succinimidyl)uranium tetrafluoroborate (18 mg, 0.060 mmol) was flushed with argon before adding anhydrous DMF (0.2 mL) and DIPEA (21 □L, 15 mg, 0.12 mmol). The reaction was stirred overnight. The solvent was removed on the lyophilizer and the crude product separated by HPLC. (7 mg, 42% yield); H NMR (DMSO-d₆, 500 MHz): δ 8.07 (2H, d, J=8.5 Hz), 7.91 (1H, d, J=6 Hz), 7.87 (1H, dd, J_{AB}=4.5 Hz, J_{AX}=9 Hz), 7.78 (1H, t, J=5.5 Hz), 7.73 (1H, dd, J_{AB}=2.5 Hz, J_{AX}=8 Hz), 7.36 (1H, td, J_{AB}=2.5 Hz, J_{AX}=9 Hz), 7.07 (2H, d, J=9 Hz), 6.23 (1H, s), 4.39 (2H, t, J=7 Hz), 4.32 (1H, q, J=6.5 Hz), 3.83 (3H, s), 3.00 (2H, m), 2.81 (1H, dd, J_{AB}=4.5 Hz, J_{AX}=13.5 Hz), 2.72 (1H, dd, J_{AB}=8 Hz, J_{AX}=14 Hz), 2.1-2.25 (4H, m), 1.78 (7H, s), 1.57 (2H, m), 1.32-1.49 (7H, m); ¹³C NMR (125 MHz, DMSO-d₆) δ 191.87, 182.61, 179.11, 177.95, 171.64, 170.17, 168.88, 165.78, 162.48, 160.86, 160.54, 145.49 (d, J=9.3 Hz), 137.32, 128.09, 125.48, 124.52, 115.48 (d, J=22.5 Hz), 114.68, 110.60 (d, J=21.2 Hz), 91.07, 69.76, 55.36, 51.98, 51.46, 51.04, 38.791, 37.81, 35.00, 29.73, 27.93, 26.00, 25.40, 24.77, 24.46, 21.43; ESI-TOF-MS (m/z): [M]⁻ calcd for [C₄₀H₄₄FN₄O₁₂S]⁻ 823.2666, found 823.2702

(253): A flask equipped with stir bar containing 250 (54 mg, 0.093 mmol) and the photocleavable cysteic acid sidechain (44 mg, 0.108 mmol) was flushed with argon before adding anhydrous DMF (1 mL) and NMM (51 μL, 47 mg, 0.463 mmol). The reaction was stirred overnight. The solvent was removed on the lyophilizer and the crude product purified by HPLC. (14 mg, 30% yield); H NMR (DMSO-d₆, 500 MHz): δ 8.07 (2H, d, 9 Hz), 7.97 (2H, m), 7.85 (1H, dd, J_{AB}=4 Hz, J_{AX}=9 Hz), 7.73 (1H, J_{AB}=2.5 Hz, J_{AX}=8 Hz), 7.54 (1H, s) 7.34-7.37 (2H, m), 7.06 (2H, d, J=8.5 Hz), 6.22 (1H, s), 5.23 (1H, q, 5.5 Hz), 4.34-4.40 (4H, m), 4.02 (2H, t, J=7 Hz), 3.91 (3H, s), 3.83 (3H, s), 3.40 (2H, quintet, J=7 Hz), 2.81-2.85 (1H, m), 2.72-2.76 (1H, m), 2.68 (1H, s), 2.11 (1H, m), 1.70-1.80 (8H, m), 1.50-1.60 (2H, m), 1.41-1.43 (2H, m), 1.35 (3H, d, J=6 Hz) ¹³C NMR (125 MHz, DMSO-d₆) δ 191.71, 182.65, 179.12, 177.95, 171.70, 171.26, 165.74, 162.50, 160.89, 160.55, 153.55, 146.04, 145.77, 145.50 (d, J=9 Hz), 138.90, 138.27, 137.30, 128.13, 124.66, 124.49, 115.50 (d, J=26 Hz), 114.68, 110.63 (d, J=25 Hz), 109.14 (d, J=36 Hz), 91.104, 67.26, 63.89, 67.26, 63.89, 56.06, 55.37, 51.83, 51.47, 50.99, 45.12, 37.99, 34.99, 27.00, 25.43, 25.07, 24.77, 24.45; ESI-TOF-MS (m/z): [M]⁻ calcd for [C₄₂H₄₆FN₄O₁₃S]⁻ 865.2761. found 865.2712

(254): A flask wrapped in aluminum foil containing 253 (15 mg, 0.017 mmol) and 4-nitrophenylchloroformate (3.4 mg, 0.017 mmol) was flushed with argon before adding DCM (170 μL) and pyridine (1.4 μL, 1.3 mg, 0.017 mmol). The reaction was stirred for 6 h at room temperature. The solvent was removed in vacuo and the crude product purified by HPLC.

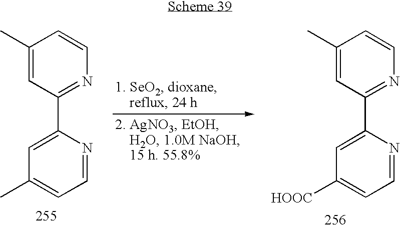

4-carboxy-4′-methyl 2,2′-bipyridine Auxiliary Ligand (255) 4,4′-dimethyl 2,2′-bipyridine (5.0 g, 27.1 mmol, 1.0 eq) was suspended in 1,4 dioxane (295 mL) with selenium dioxide (3.61 g, 32.6 mmol, 1.2 eq). The solution was heated at reflux for 25 h with stirring. The solution turned yellow and then black. The solution was then filtered hot through celite and the solvent was removed in vacuo. Ethanol (150 mL) was added and silver nitrate (4.31 g, 25.3 mmol, 1.1 eq in 40 mL H2O). 1M NaOH (100 mL) was added dropwise over 30 m while stirring under argon vigorously. The solution was allowed to stir for 24 h. The ethanol was removed in vacuo and the aqueous residue filtered. The solid was washed with 1.3M NaOH (2×30 mL) and extracted with DCM (4×100 mL). 1:1 (v/v) 4.0 N HCl/acetic acid was added to pH=3.5. A white solid ppt. formed which was filtered and dried. The solid was continuously extracted with acetone in a Soxhlet for 140 h. The solution was then cooled and the solvent removed in vacuo to give 3.244 g of off white solid (55.8%). ¹H NMR (500 MHz, CDCl₃) δ 9.16 (s, 1H), 8.99 (d, 1H, J=5.5 Hz), 8.93-8.94 (d, 1H, J=5 Hz), 8.48 (s, 1H), 8.04-8.05 (d, 1H, J=5 Hz), 7.61 (s, 1H), 2.69 (s, 3H). ¹³C NMR (500 MHz, DMSO) δ 20.70, 119.69, 121.29, 123.06, 125.30, 148.21, 149.18, 150.05, 154.44, 156.27, 157.53, 166.57.

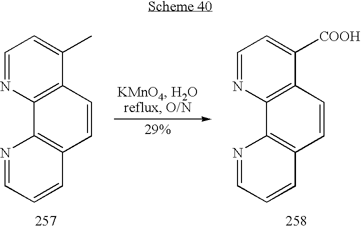

### 4-carboxy-1,10-phenanthroline Auxiliary ligand (257)

4-methyl-1,10-phenanthroline (1 g, 5.15 mmol, 1 eq) and KMnO₄ (3.25 g, 20.6 mmol, 4 eq) were stirred in 30 mL of H₂O at reflux for 23 h. The solution was filtered hot through celite and the resulting solution was evaporated in vacuo. The resulting solid was recrystallized from H₂O to yield white solid which was filtered and washed with white solid. The eluent was concentrated and recrystallized further to yield 331 mg of white product (29%). ¹H NMR (3500 MHz, DMSO) 8.27 (t, 1H, J=6.3 Hz), 8.37 (s, 1H), 8.38 (d, 1H, J=5.0 Hz), 8.91 (d, 1H, J=9.3 Hz). 9.17 (d, 1H, J=7.5 Hz), 9.30 (d, 1H, J=4.8 Hz), 9.38 (d, 1H, J=4.5 Hz). δ ¹³C NMR (500 MHz, DMSO) δ 125.83, 126.11, 126.18, 126.79, 129.41, 138.39, 141.00, 144.45, 146.64, 150.91, 167.11. HRMS m/z (ESI-TOF) for C₁₃H₈N₂O₂ Calculated 224.0586. Found 225.0645.

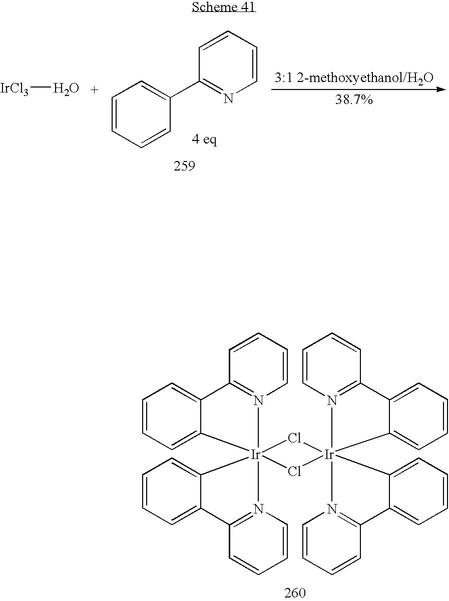

Dichloro Iridium dimer (260) To a solution of Iridium trichloride monohydrate (1 g, 3.35 mmol, 1 eq) in 2-methoxyethanol (30 mL) and water (10 mL), 2-phenyl pyridine (1.2 mL, 8.37 mmol, 2.5 eq) was added and refluxed for 24 h. at 125-130° C. The solution was cooled and the ppt. was filtered and washed with ethanol (60 mL) and acetone (60 mL). The solid was then dissolved in 250 mL DCM and filtered. 55 mL of toluene and 25 mL of hexanes were added and evaporated to approximately 150 mL. No ppt. was formed. The solvent was removed in vacuo and the compound purified by column chromatography (10:1 DCM/MeOH) to give 692.9 mg of yellow product (38.7%). R_{f}=0.81 (6:1 DCM/MeOH). ¹H NMR (500 MHz, CDCl₃) δ 5.93-5.95 (d, 4H, J=8 Hz), 6.55-6.58 (t, 4H, J=7 Hz), 6.74-6.79 (2t, 8H, J=7.5 Hz, 6 Hz), 7.53-7.54 (d, 4H, J=7.5 Hz), 7.73-7.76 (td, 4H, J=1 Hz, 8.5 Hz), 7.91-7.93 (d, 4H, J=8 Hz), 9.24-9.25 (d, 4H, J=5.5 Hz). ¹³C NMR (500 MHz, CDCl₃) δ 118.63, 121.55, 122.34, 123.88, 129.32, 130.82, 136.39, 151.92.

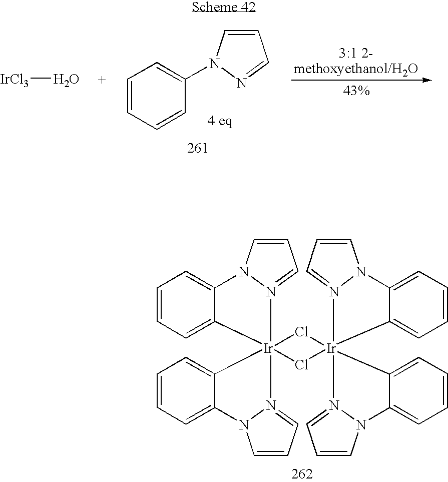

Synthesis of Blue dimer (262) 1-phenyl pyrazole (1.11 mL, 8.37 mmol, 2.5 eq) was added dropwise to a solution of IrCl₃H₂O (1 g, 3.35 mmol, 1 eq) in 2-methoxyethanol (36 mL) and water (12 mL) under argon. The solution was refluxed at 110° C. for 48 h. No color change was noted. The solution was cooled and filtered. The solid was washed with ethanol and acetone but dissolved. The solvent was removed in vacuo to give 744.1 mg of white solid (43%). R_{f}=0.83 (6:1 DCM/MeOH). ¹H NMR (500 MHz, DMSO) δ 5.80-5.81 (dd, 2H, J=0.5 Hz, 7 Hz), 6.18-6.19 (d, 2H, J=7.5 Hz), 6.64-6.71 (t, td, 4H, J=8 Hz, 1 Hz, 7.5 Hz), 6.81-6.82 (t, 2H, J=2.5 Hz), 6.85-6.89 (td, 2H, J=1 Hz, 7.5 Hz), 6.90-6.94 (td, 2H, J=1 Hz, 7 Hz), 6.95-6.96 (t, 2H, J=2.5 Hz), 7.55-7.56 (d, 2H, J=8 Hz), 7.60-7.61 (d, 2H, J=7.5 Hz), 8.10-8.11 (d, 2H, J=2 Hz), 8.45-8.46 (d, 2H, J=2 Hz), 8.80-8.81 (d, 2H, J=2.5 Hz), 8.93-8.94 (d, 2H, J=3 Hz). ¹³C NMR (500 MHz, DMSO) δ 107.35, 108.62, 111.12, 111.88, 122.65, 122.84, 125.17, 125.88, 127.50, 127.72, 128.97, 131.81, 132.65, 135.48, 138.94, 139.87, 141.80, 142.51. HRMS m/z (ESI-TOF) for C₁₈H₁₄IrN₄⁻ Calculated 479.08. Found 479.0855.

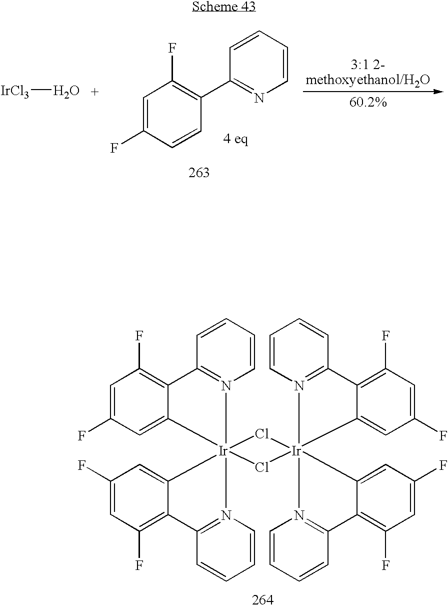

### Synthesis of Green Dimer (264)

2-(2,4-difluorophenyl)pyridine (1.28 mL, 8.37 mmol, 2.5 eq) was added dropwise to a solution of IrCl₃H₂O (1 g, 3.35 mmol, 1 eq) in 2-methoxyethanol (36 mL) and water (12 mL) under argon. The solution was refluxed at 110° C. for 48 h. The color changed to bright yellow. The solution was cooled and the solid was purified by column chromatography (10:1 DCM/MeOH). The solvent was removed in vacuo to give 1.23 g of yellow solid (60.2%). R_{f}=0.81 (6:1 DCM/MeOH). ¹H NMR (500 MHz, DMSO) δ 5.207-5.22 (d, 2H, J=8.5 Hz), 5.29-5.31 (d, 2H, J=8 Hz), 6.33-6.37 (t, 4H, J=10 Hz), 6.82-6.85 (t, 4H, J=6.5 Hz), 7.82-7.85 (t, 4H, J=9 Hz), 8.31-8.47 (d, 4H, J=8.5 Hz), 9.12-9.13 (d, 4H, J=5.5 Hz). HRMS m/z (ESI-TOF) for C₂₂H₁₂F₄IrN₂⁻ Calculated 573.05. Found 573.0553.

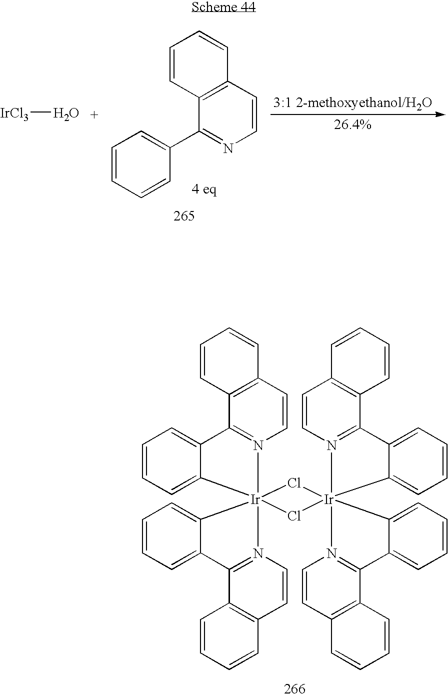

### Synthesis of Red Dimer (266)

1-phenyl isoquinoline (425 mg, 2.07 mmol, 2.5 eq) was added to a solution of IrCl₃H₂O (0.25 g, 0.837 mmol, 1 eq) in 2-methoxyethanol (9 mL) and water (3 mL) under argon. The solution was refluxed at 110° C. for 19 h. The color changed to red. The solution was cooled and the solid filtered and washed with ethanol (50 mL) and acetone (50 mL). The solid was then purified by column chromatography (10:1 DCM/MeOH). The solvent was removed in vacuo to give 14.1 mg of yellow solid (26.4%). R_{f}=0.71 (6:1 DCM/MeOH). ¹H NMR (500 MHz, DMSO) δ 6.02-6.04 (d, 1H, J=7.5 Hz), 6.34-6.36 (t, 2H, J=6 Hz), 6.56-6.56 (d, 1H, J=6.5 Hz), 6.82-6.79 (t, 2H, J=7.5 Hz), 7.50-7.57 (p, 2H, J=7.5 Hz), 7.68-7.72 (q, 2H, J=7.5 Hz), 7.74-7.77 (t, 4H, J=7.5 Hz), 7.81-8.11 (m, 4H), 8.11-8.13 (d, 2H, J=8 Hz), 8.96-8.98 (d, 1H, J=9 Hz), 9.04-9.06 (d, 1H, J=6 Hz). ¹³C NMR (500 MHz, CDCl₃) δ 119.72, 121.18, 126.29, 127.15, 127.33, 127.69, 128.59, 129.13, 129.70, 130.13, 130.93, 131.6, 131.69, 143.97, 149.58. HRMS m/z (ESI-TOF) for C₃₀H₂₀IrN₂⁻ Calculated 601.12. Found 601.1236.

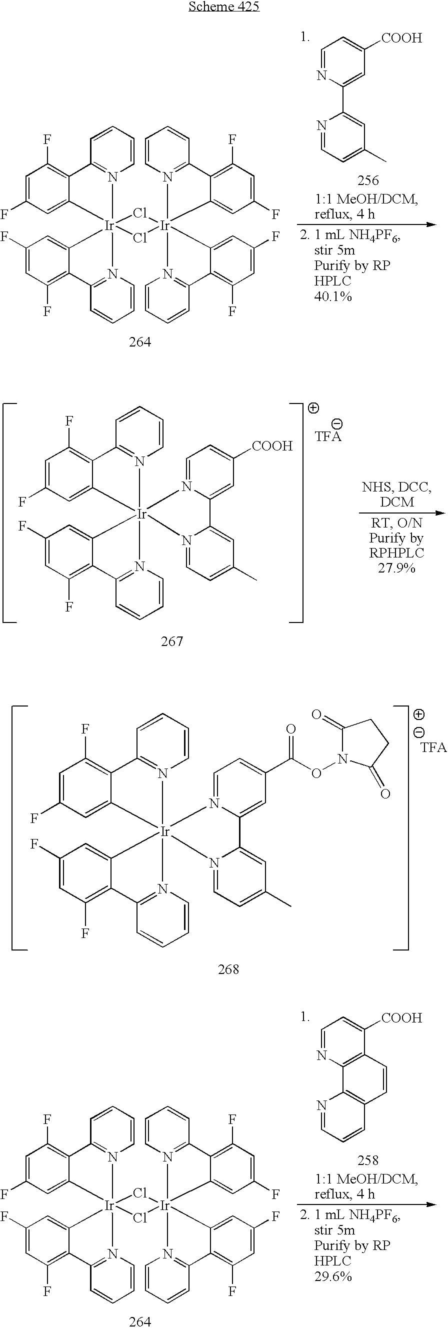

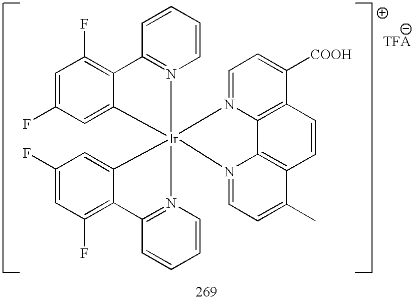

Iridium Complex (267) Iridium dimer 264 (114 mg, 0.094 mmol, 1 eq) and 4-carboxy-4′-methyl bipyridine ligand 256 (43.0 mg, 0.200 mmol, 2.1 eq) were stirred in a 1:1 mixture of anhydrous DCM/MeOH (20 mL) at reflux (50-60° C.) under argon for 4 h. The solution was then cooled to RT and 1.0 mL of sat. NH₄PF₆^{in MeOH} were added while stirring. The solution was allowed to stir for 5 min. The solvent was evaporated under vacuum and the resulting solid was purified with RP HPLC (50-80% B, 35 min, 254 nm, 20 mL/min, t_{R}=12.8 min) to give 67.7 mg of yellow solid (40.1%). R_{f}=0.075 (10:1 DCM/MeOH). ¹H NMR (^{d}DMSO, 500 MHz) δ 2.52 (s, 3H), 5.56 (qd, 2H, J=2 Hz, 8.5 Hz), 6.95 (m, 2H), 7.19 (d, t, 2H, J=2.2 Hz, 7H), 7.53 (d, 1H, J=5.5 Hz), 7.66 (d, 1H, J=6 Hz), 7.72 (d, 2H, J=5.5 Hz), 8.01 (p, 2H, J=7.5 Hz), 8.03 (s, 2H), 8.25 (t, 2H, J=6.5 Hz), 9.03 (s, 1H), 9.12 (s, 1H).

Activated Green Complex 268: Iridium complex 267 (100 mg, 0.109 mmol, 1 eq), DCC (135.3 mg, 0.656 mmol, 6 eq), and NHS (100.6 mg, 0.875 mmol, 8 eq) were stirred in anhydrous DCM (10 mL) under argon at RT for 3 h. The solvent was removed under reduced pressure and the resulting orange solid was purified by RP HPLC (50-100% B, 20 mL/min, 254 nm, 40 min, t_{R}=14 min) to give a yellow/orange solid 61.4 mg (56.3%). ¹H NMR (300 MHz, DMSO) δ 2.59 (s, 3H), 2.95 (s, 4H), 5.60-5.64 (t, d, 2H, J=2.1 Hz, 8.4 Hz), 6.95-7.03 (td, 2H, 2H, J=3 Hz, 9.9 Hz), 7.18-7.27 (p, 2H, 6.9 Hz), 7.56-7.61 (t, 1H, J=9.3 Hz), 7.68-7.70 (d, 1H, J=5.4 Hz), 7.75-7.78 (dd, 1H, J=1.8 Hz, 7.5 Hz), 7.80-7.86 (dd, 1H, J=5.7 Hz, 20.4 Hz), 8.05-8.07 (d, 4H, J=7.5 Hz), 8.19-8.23 (q, 1H, J=5.7 Hz), 8.28-8.31 (d, 2H, J=7.2 Hz), 9.13-9.16 (d, 1H).

### Green Iridium Dye (269)

Iridium dimer 264 (110.8 mg, 0.0822 mmol, 1 eq) and phenanthroline ligand 258 (47.7 mg, 0.164 mmol, 2 eq) were stirred in a solution of 1:1 DCM/MeOH (14 mL) under argon at reflux (50-60° C.) for 4 h. The solution was then cooled to RT and 1 mL of concentrated NH₄PF₆ in MeOH was added and stirred for 5 min. The solvent was removed under vacuum and the resulting solid was purified by RP HPLC (60-100% B, 40 min, 20 mL/min, 254 nm) to yield 24.5 mg of yellow solid (29.6%). R_{f}=0.79 (6:1 DCM/MeOH). ¹H NMR (^{d}DMSO, 500 MHz) δ 5.64 (td, 2H, J=2 Hz, 8.5 Hz), 7.02 (m, 4H), 7.47 (d, 1H, J=5.5 Hz), 7.58 (d, 1H, J=6 Hz), 7.95 (t, 2H, J=7.5 Hz), 7.58 (d, 1H, J=6 Hz), 7.95 (t, 2H, J=7.5 Hz), 8.05 (m, 1H), 8.25 (d, 2H, J=9 Hz), 8.30 (d, 1H, J=5 Hz), 8.34 (d, 1H, J=5 Hz), 8.40 (d, 1H, J=5 Hz), 8.46 (d, 1H, J=9 Hz), 8.94 (d, 1H, J=8.5 Hz), 8.98 (d, 1H, J=9.5 Hz). ¹³C NMR (500 MHz, DMSO) δ 99.49, 99.69, 99.89, 113.74, 123.69, 123.83, 124.92, 126.30, 128.27, 128.32, 129.27, 129.99, 131.45, 139.11, 139.70, 140.43, 146.13, 147.30, 150.40, 150.63, 152.63, 152.06, 154.18, 154.38, 158.30, 158.56, 160.07, 160.17, 162.27, 163.15, 164.45, 166.52. HRMS m/z (ESI-TOF) for C₃₅H₂₀F₄IrN₄O₂ Calculated 797.1152. Found 797.1168.

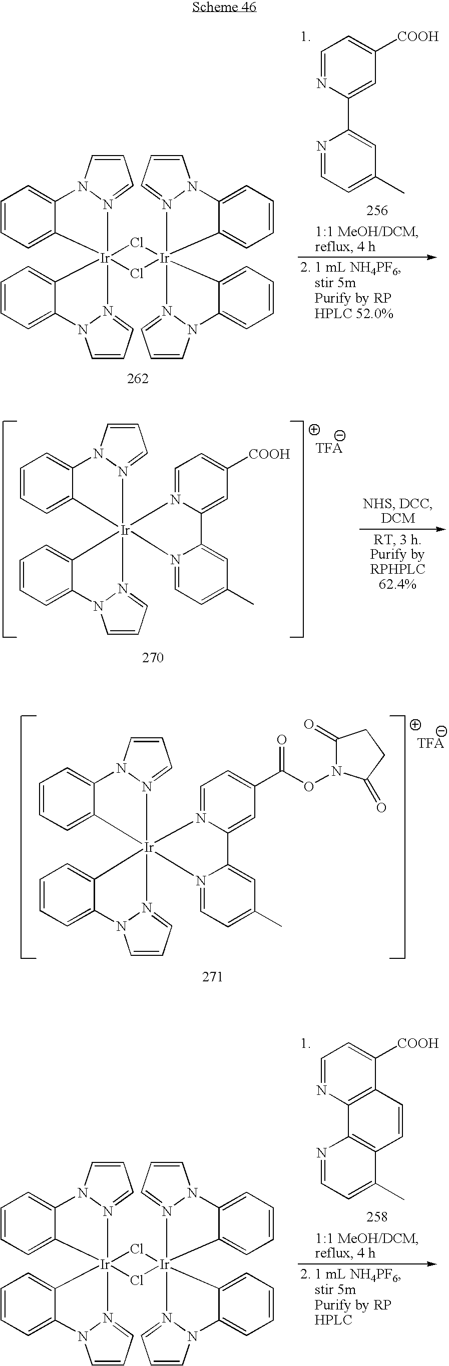

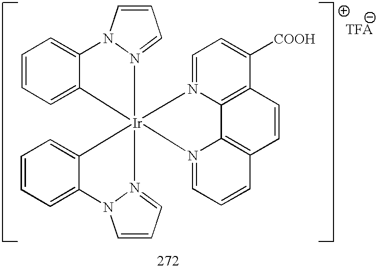

Iridium Complex (270) Iridium dimer 262 (155 mg, 0.151 mmol, 1 eq) and 4-carboxy-4′-methyl bipyridyl ligand 256 (65.6 mg, 0.306 mmol, 2 eq) were stirred in a 1:1 mixture of anhydrous DCM/MeOH (20 mL) at reflux (50° C.) under argon for 4.5 h. The solution was then cooled to RT and 1.0-1.2 mL of sat. NH₄PF₆ in MeOH was added while stirring. The solution was allowed to stir for 5 min. The solvent was evaporated under vacuum and the resulting solid was purified with RP HPLC (30-80% B, 35 min, 254 nm, 20 mL/min, t_{R}=11.1 min) to give 126.5 mg orange solid (52.0%). R_{f}=0.10 (10:1 DCM/MeOH). ¹H NMR (^{d}DMSO, 500 MHz) δ 2.49 (s, 3H), 6.16 (1H, J=7 Hz), 6.18 (d, 1H, J=7.5 Hz), 6.68 (p, 2H, J=3 Hz), 6.85 (m, 2H), 7.03 (m, 2H), 7.19 (d, 1H, J=2 Hz), 7.30 (d, 1H, J=2.5 Hz), 7.54 (d, 1H, J=5.5 Hz), 7.68 (t, 2H, J=7 Hz), 7.83 (d, 1H, J=6 Hz), 8.06 (dd, 1H, J=1 Hz, 5.5 Hz), 8.17 (d, 1H, J=5.5 Hz), 8.88 (dd, 2H, J=3 Hz, 8 Hz), 9.00 (s, 1H) 9.12 (s, 1H). ¹³C NMR (^{d}DMSO, 500 MHz) δ 20.57, 108.35, 108.42, 111.99, 122.90, 123.45, 125.91, 126.24, 126.30, 127.28, 128.50, 128.57, 128.99, 132.12, 132.29, 132.49, 138.73, 138.90, 140.82, 142.72, 142.86, 149.32, 151.26, 151.76, 154.86, 157.16, 164.79, 176.61, 181.37.

Activated Blue complex (271) A mixture of Iridium complex 270 (100 mg, 0.122 mmol, 1 eq), NHS (150.8 mg, 0.731 mmol, 6 eq), and DCC (150.8 mg, 0.731 mmol, 6 eq) was stirred in anhydrous DCM (10 mL) under argon at RT for 3 h. The solvent was removed under reduced pressure to yield an orange solid, which was purified by RP HPLC (50-100%, 20 mL.min, 254 nm, 40 min, t_{R}=12 m) to yield 68.6 mg of orange solid (62.4%). Rf=0.54 (6:1 DCM/MeOH) ¹H NMR (300 MHz, DMSO) δ 2.52 (s, 3H), 2.88 (s, 4H), 6.09-6.13 (t, 2H, J=7.5 Hz), 6.62 (s, 2H), 6.77-6.80 (t, 2H, J=7.2 Hz), 6.95-7.00 (t, 2H, J=7.2 Hz), 7.13 (s, 1H), 7.38 (s, 1H), 7.46-7.51 (t, 1H, J=5.4 Hz), 7.60-7.65 (t, 2H, J=6.9 Hz), 7.76-7.80 (t, 1H, J=5.4 Hz), 8.15-8.24 (dd, 2H, J=5.7 Hz, 21.3 Hz), 8.81-8.83 (dd, J=3.3 Hz, 8.1 Hz), 8.66 (s, 1H), 8.81 (s, 1H). ¹³C NMR (300 MHz, DMSO) δ 20.72, 25.59, 123.04, 123.58, 124.09, 126.04, 126.39, 127.38, 127.62, 128.69, 129.09, 129.41, 132.03, 132.38, 132.63, 134.26, 138.99, 139.37, 141.14, 142.79, 142.86, 149.56, 151.37, 151.95, 152.10, 154.55, 155.02, 157.28, 158.07, 158.41, 159.98, 164.87, 169.82, 172.76.

Blue Complex (272) Iridium dimer 262 (203 mg, 0.195 mmol, 1 eq) and phenanthroline ligand 258 (87.2 mg, 0.389 mmol, 2 eq) were stirred in a solution of 1:1 DCM/MeOH (20 mL) under argon at reflux (60° C.) for 4 h. The solution was cooled to RT and 1 mL of concentrated NH4PF6 was added and stirred for 10 min. The solution was rotovapped and the resulting orange solid was purified by RP HPLC (60-90% B, 40 min, 20 mL/min, 254 nm) to yield an orange solid. Rf=TBD (6:1 DCM/MeOH). ¹H NMR (500 MHz, DMSO) δ 6.249 (t, 2H, J=7.5 Hz), 6.549 (s, 2H), 6.848 (t, 2H, J=7.5 Hz), 7.050 (p, d, 3H, J=3.5 Hz, 2.5 Hz), 7.196 (d, 1H, J=2 Hz), 7.681 (d, 2H, J=8 Hz), 8.013 (q, 1H, J=5.5 Hz), 8.334 (t, 2H, J=5 Hz), 8.412 (d, 1H, J=9 Hz), 8.454 (d, 1H, J=5 Hz), 8.826 (t, 2H, J=3 Hz), 8.869 (d, 1H, J=8 Hz), 8.949 (d, 1H, J=9 Hz). ¹³C NMR (500 MHz, DMSO) δ 108.87, 109.90, 112.58, 113.31, 123.65, 125.59, 125.97, 126.84, 127.11, 127.52, 127.67, 128.92, 129.06, 129.69, 130.00, 131.12, 132.08, 132.22, 132.38, 133.06, 133.14, 138.78, 139.16, 139.55, 139.76, 141.43, 141.65, 143.47, 143.56, 147.16, 148.27, 151.69, 151.76, 158.57, 158.84, 159.18, 166.64.

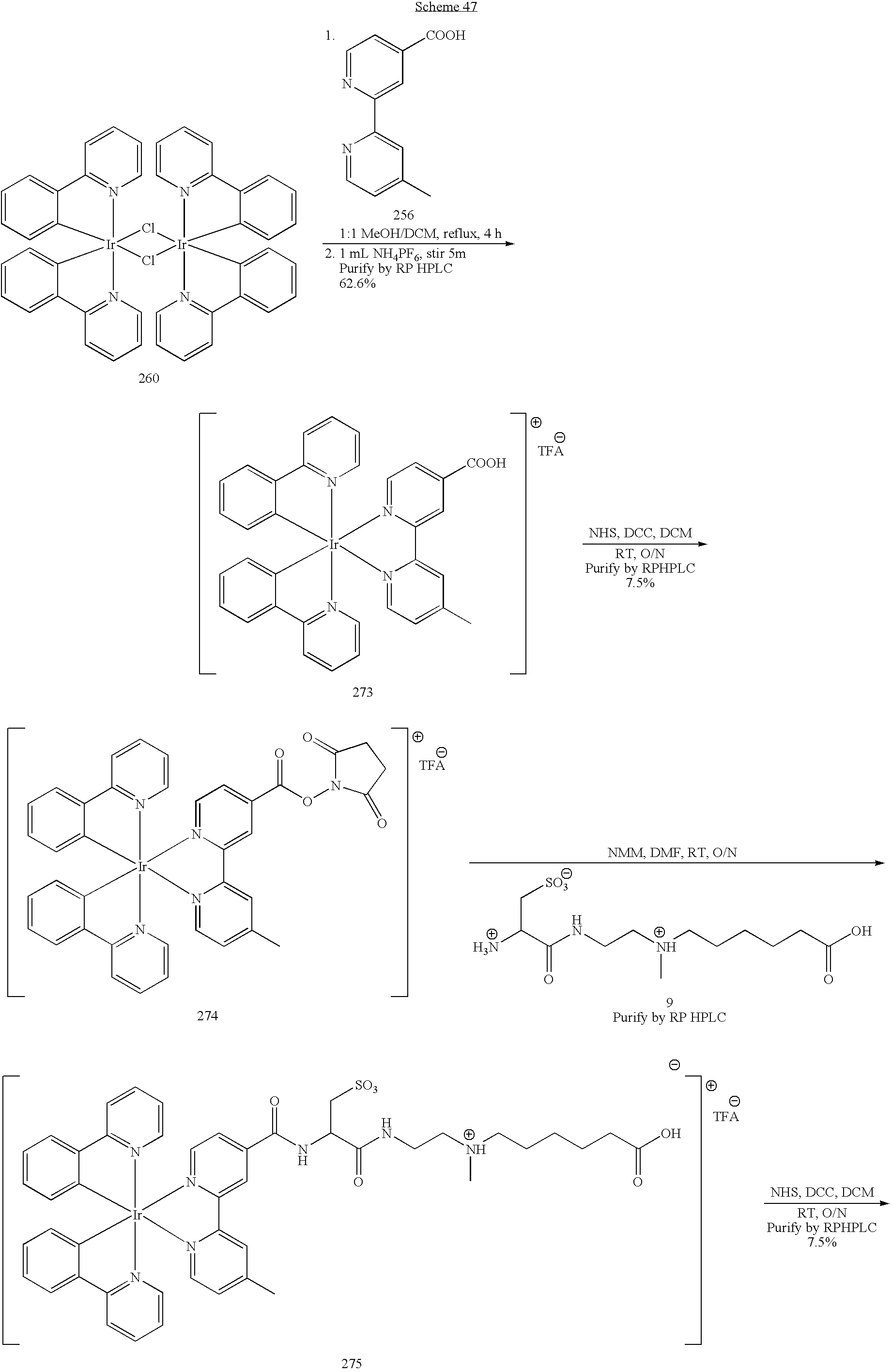

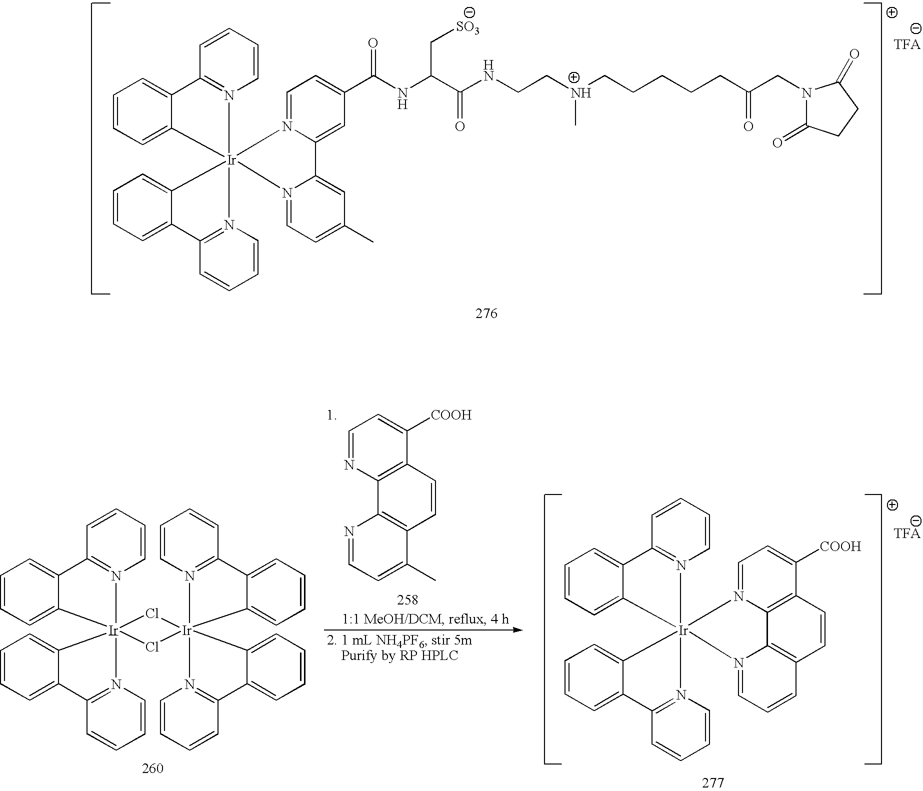

Iridium Complex (273) Iridium dimer 260 (150 mg, 0.140 mmol, 1 eq) and 4-carboxy-4′-methyl bipyridyl ligand 258 (60.0 mg, 0.280 mmol, 2 eq) were stirred in a 1:1 mixture of anhydrous DCM/MeOH (20 mL) at reflux (50-60° C.) under argon for 4 h. The solution was then cooled to RT and 1.0 mL of sat. NH₄PF₆ ^{in MeOH} was added while stirring. The solution was allowed to stir for 5 min. The solvent was evaporated under vacuum and the resulting solid was purified with RP HPLC (60-75% B, 30 min, 254 nm, 20 mL/min, t_{R}=8.85 min) to give 174.0 mg of orange solid (62.6%). R_{f}=0.12 (10:1 DCM/MeOH). ¹H NMR (^{d}DMSO, 500 MHz) δ 2.50 (s, 3H), 6.87 (tt, 2H, J=3 Hz, 4.5 Hz), 6.98 (tt, 2H, J=3.5 Hz), 7.10 (tt, 2H, J=7 Hz, 22 Hz), 7.51 (d, 1H, J=6 Hz), 7.57 (d, 1H, J=6 Hz), 7.64 (q, 2H, J=6 Hz), 7.89 (m, 4H), 7.99 (d, 1H, J=5.5 Hz), 8.03 (dd, 1H, J=1.2 Hz, 7 Hz), 8.23 (t, 2H, J=8.5 Hz), 8.99 (s, 1H), 9.11 (s, 1H). Mp=121-122° C. ¹H NMR (500 MHz, CDCl₃) δ 2.83 (s, 3H), 6.28-6.30 (dd, 2H, J=2.5 Hz, 7.5 Hz), 6.91-7.11 (q, p, q, 6H, J=7.5 Hz, 7 Hz, 7 Hz), 7.20-7.22 (d, 2H, J=5.5 Hz), 7.44-7.53 (dd, 2H, J=5.5 Hz, 14 Hz), 7.67-7.70 (d, 2H, J=7 Hz), 7.69-7.76 (t, 2H, J=2.5 Hz), 7.91-7.93 (q, 2H, J=4 Hz), 7.97-8.02 (q, 2H, J=5.5 Hz), 8.86 (s, 1H), 9.50 (s, 1H). ¹³C NMR (500 MHz, CDCl₃) δ 21.70, 119.85, 120.03, 122.97, 123.18, 123.40, 123.57, 125.03, 125.20, 126.07, 128.32, 129.28, 131.11, 131.28, 131.93, 138.36, 138.47, 143.53, 143.54, 147.66, 148.44, 148.69, 150.00, 150.01, 150.29, 151.20, 152.38, 155.53, 161.38, 168.06. HRMS m/z (ESI-TOF) for ^{C}₃₄^{H}₂₆^{IrN}₄^{O}₂⁺ Calculated 715.168. Found 715.16812.

NHS Activated Cationic Iridium Complex (274) Iridium complex 273 (67.6 mg, 0.079 mmol, 1 eq), N-hydroxysuccinimide (72.4 mg, 0.629 mmol, 8 eq), and 1,3-dicyclohexylcarbodiimide (97.3 mg, 0.472 mmol, 6 eq) were stirred in a solution of anhydrous DCM (3 mL) under argon overnight. The solvent was removed in reduced pressure and the resulting orange solid purified by RP HPLC (50-100% B, 40 min, 20 mL/min, 254 nm, t_{R}=13 min) to yield 21 mg of orange solid (27.9%). R_{f}=0.5 (6:1 DCM/MeOH). ¹H NMR (500 MHz, CDCl₃) δ 2.76 (s, 4H), 2.95 (s, 3H), 6.28-6.32 (t, 2H, J=7 Hz), 6.95-6.70 (m, 2H), 7.06-7.08 (t, 6H, J=6.5 Hz), 7.46-7.47 (d, 1H, J=3 Hz), 7.55-7.57 (d, 2H, J=8.5 Hz), 7.70-7.71 (d, 2H, J=7.5 Hz), 7.80-7.81 (d, 2H, J=5 Hz), 7.93-7.94 (d, 1H, J=6.5 Hz), 7.97-7.98 (d, 1H, 5.5 Hz), 8.02-8.05 (q, 1H, J=6 Hz), 8.18-8.19 (d, 1H, 5.5 Hz), 8.74 (s, 1H), 9.47 (s, 1H).

Zwitterionic Side Chain attachment (275) Activated complex 274 (21 mg, 0.022 mmol, 1 eq), N-methylmorpholine (0.048 mL, 0.439 mmol, 20 eq), and peptide side chain 9 (59.7 mg, 0.132, 6 eq) were stirred in DMF (3 mL) under argon at RT overnight. The solvent was then removed under reduced pressure and the solid purified by RP HPLC (40-100% B, 20 mL/min, 254 nm) to yield a yellow solid. R_{f}=0.69 (6:1 DCM/MeOH) ¹H NMR (500 MHz, CDCl₃) δ 1.25 (m, 2H), 1.52 (m, 1H), 1.61 (m, 1H), 1.80 (m, 2H) 2.37 (m, 2H), 2.63 (s, 3H), 2.93 (s, 3H), 2.97 (m, 2H), 3.16 (m, 2H), 3.36 (m, 2H), 3.87 (m, 2H), 4.11 (m, 1H), 4.29 (m, 1H), 5.22 (m, 1H), 6.29-6.30 (m 2H), 6.92-7.09 (m, 6H), 7.47 (s, 1H), 7.56 (m, 1H), 7.67-7.70 (m, 2H), 7.74-7.76 (m, 32H), 7.88-7.92 (m, 32H), 8.00-8.02 (m, 1H), 9.31-9.37 (m, 2H), 9.61-9.66 (mb, 1H). HRMS m/z (ESI-TOF) for C₄₆H₄₉O₇N₇IrS⁺ Calculated 1036.30. Found 1036.3013.

Iridium Z-Dye (276) Zwitterionic iridium complex 275 (33.3 mg, 0.0354 mmol, 1 eq), NHS (32.6 mg, 0.0283 mmol, 8 eq), and DCC (43.8 mg, 0.212 mmol, 6 eq) were stirred in anhydrous DCM (5 mL) under argon at RT overnight. The resulting yellow solid was purified by RP HPLC (40-100% B, 4 mL/min, 254 nm) and the fractions were evaporated under reduced pressure to give 2.71 mg yellow solid (7.5%). Rf=0.34 (6:1 DCM/MeOH). ¹H NMR (500 MHz, CDCl₃) δ 1.46 (m, 2H), 1.62-1.64 (m, 2H), 1.74-1.78 (m, 4H), 1.80 (m, 2H), 2.59 (s, 3H), 2.88 (s, 3H), 2.93 (m, 4H), 3.01 (m, 2H), 3.15-3.20 (m, 2H), 3.33 (m, 4H), 3.54 (m, 1H), 3.84 (m, 1H), 5.10 (m, 1H), 6.29-6.30 (q, 2H, J=7.5 Hz), 6.92-6.94 (t, 2H, J=7.5 Hz), 7.04-7.07 (t, 4H, J=7.5 Hz), 7.22-7.23 (d, 1H, J=4.5 Hz), 7.48 (m, 1H), 7.54 (m, 1H), 7.69-7.70 (d, 2H, J=7.5 Hz), 7.75-7.79 (m, 3H), 7.90-7.92 (d, 3H, J=7.5 Hz), 8.02 (s, 1H), 8.95 (m, 2H), 9.58 (m, 1H). HRMS m/z (ESI-TOF) for IrC₅₀H₅₂N₈O₉S⁺ Calculated 1133.32. Found 1133.3203.

Yellow Iridium Dye (277) Iridium dimer 260 (100 mg, 0.0932 mmol, 1 eq) and phenanthroline ligand 258 (41.8 mg, 0.187 mmol, 2 eq) were stirred in a solution of 1:1 DCM/MeOH (14 mL) under argon at reflux (60° C.) for 24 h. The solution was cooled to RT and 1 mL of concentrated NH₄PF₆ was added and stirred for 20 min. The solution was rotovapped and purified by RP HPLC (50-85% B, 40 min, 20 mL/min, 254 nm) to yield a yellow solid which is still awaiting a weight analysis. Rf=0.49 (6:1 DCM/MeOH) ¹H NMR (500 MHz, DMSO) δ 6.238 (t, 2H, J=8 Hz), 6.95 (s, 4H, J=7.5 Hz), 7.02 (t, 2H, J=7.5 Hz), 7.41 (d, 1H, J=5.5 Hz), 7.51 (d, 1H, J=6 Hz), 7.84 (t, 2H, J=3 Hz), 7.92 (d, 2H, J=8 Hz), 8.05 (q, 1H, J=5 Hz), 8.22 (q, 3H, J=8.5 Hz), 8.32 (d, 1H, J=5 Hz), 8.37 (d, 1H, J=5.5 Hz), 8.44 (d, 1H, J=9 Hz), 8.90 (d, 1H, J=6.5 Hz), 8.96 (d, 1H, J=12 Hz). ¹³C NMR (500 MHz, DMSO) δ 120.42, 122.92, 124.33, 125.53, 12621, 127.96, 128.09, 129.14, 129.91, 130.70, 131.33, 131.60, 131.68, 138.68, 139.20, 144.39, 144.49, 146.48, 147.60, 149.67, 149.93, 150.17, 150.32, 151.40, 151.47, 158.31, 158.57, 166.64, 167.12, 167.24. HRMS m/z (ESI-TOF) for C₃₅H₂₄IrN₄O₂ Calculated 725.1529. Found 725.1529.

The terms “a” and “an” do not denote a limitation of quantity, but rather denote the presence of at least one of the referenced item. The term “or” or “and/or” is used as a function word to indicate that two words or expressions are to be taken together or individually. The terms “comprising”, “having”, “including”, and “containing” are to be construed as open-ended terms (i.e., meaning “including, but not limited to”). The endpoints of all ranges directed to the same component or property are inclusive and independently combinable.

All publications and patent applications herein are incorporated by reference to the same extent as if each individual publication or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law.

Moreover, any combination of the above-described elements in all possible variations thereof is encompassed by the invention unless otherwise indicated herein or otherwise clearly contradicted by context.

1. An optical labeling molecule selected from the group consisting of structural Formula (I), structural Formula (III), structural Formula (XXI), structural Formula (XV), and structural formula (XV′), or a salt or solvate thereof, 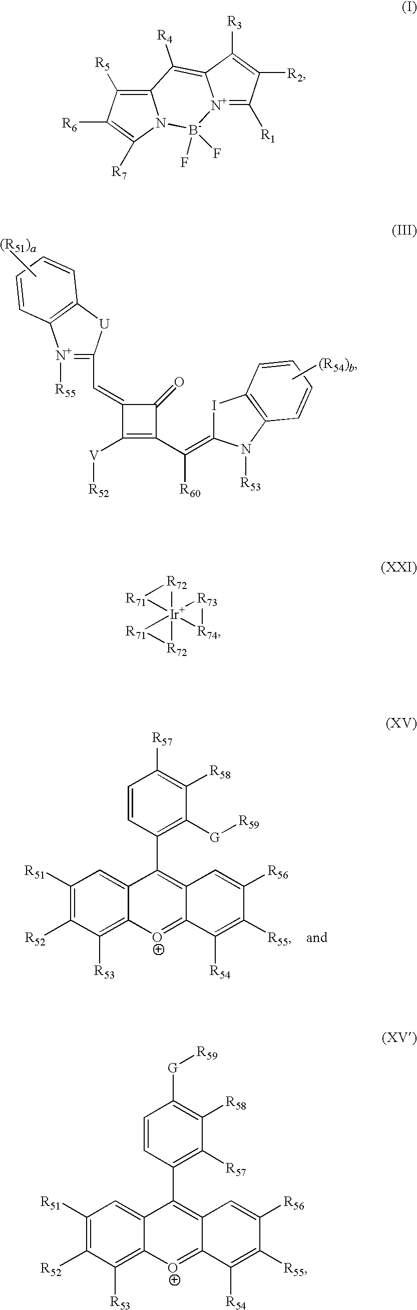
wherein the optical labeling molecule comprises a fluorophore with a derivative tail, and the derivative tail comprises at least one amide bond, wherein:
R₁ to R₇, and R₅₁ to R₅₉, are each independently hydrogen, acyl, substituted acyl, alkoxy, substituted alkoxy, alkoxycarbonyl, substituted alkoxycarbonyl, alkyl, substituted alkyl, amino, substituted amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —(CH₂)ₙN⁺(CH₃)₃, —S(O)ₜR₂₀, —SO₃H, —(CH₂)ₙS(O)ₙOH, —(CH₂)ₙS(O)₂O⁻, —OP(O)(O⁻)₂, or —(CH₂)ₙOP(O)(O⁻)₂, 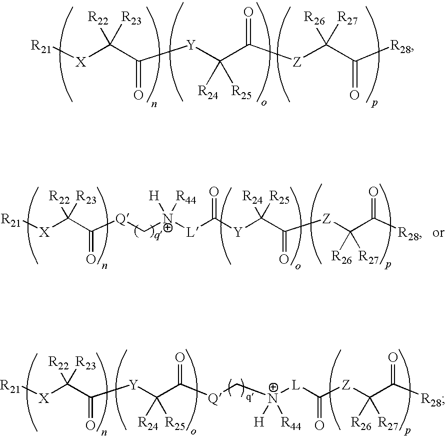
provided that one and only one of R₁ to R₇, or one and only one of R₅₂ to R₅₅, or one and only one of R₅₇ to R₅₉ is 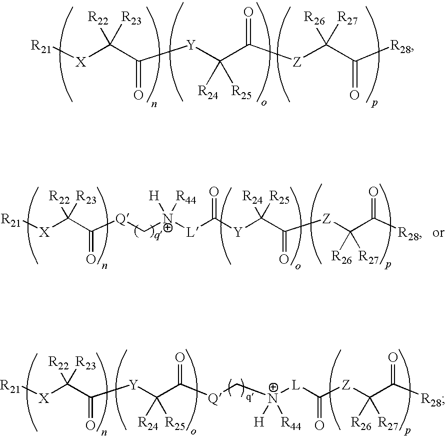
R₇₁ to R₇₄ are each independently aryl, substituted aryl, heteroaryl, or substituted heteroaryl, provided that R₇₁ to R₇₄ contain at least one amino, substituted amino, acyl, substituted acyl, 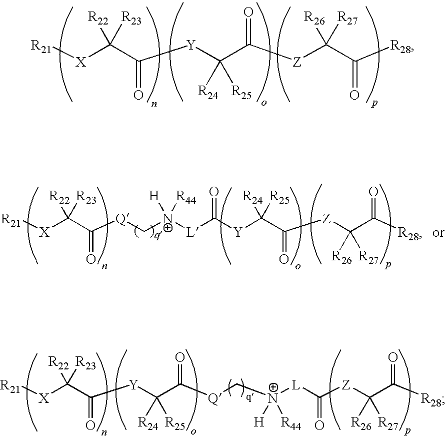
R₂₂, R₂₃, R₂₄, R₂₅, R₂₆ and R₂₇ are each independently hydrogen, alkyl, substituted alkyl, amino, substituted amino, aryl, substituted aryl, arylalkyl, substituted arylalkyl, heteroaryl, substituted heteroaryl, heteroarylalkyl, heteroalkyl or substituted heteroalkyl, nitro, —(CH₂)ₙN⁺(CH₃)₃, —S(O)ₜR₂₀, —SO₃H, —(CH₂)ₙS(O)OH, —(CH₂)ₙS(O)₂O⁻, —OP(O)(O⁻)₂, or —(CH₂)ₙOP(O)(O⁻)₂;
R₂₁ is —(CH₂)ₘ—C(O)—, —(CH₂)ₘ—C(O)-Q′(CH₂)_{q}—N⁺H(R₄₆)-L′—C(O)⁻, 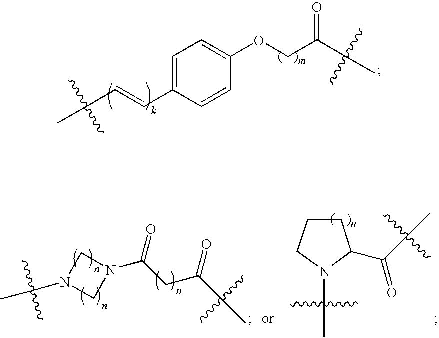
R₂₈ is -Q-L-C(O)-A; -Q(CH₂)_{q}—N⁺H(R₄₅)-L-C(O)-A, -Q-L-D-C(O)—(B′)ᵣ-A or -Q(CH₂)_{q}—N⁺H(R₄₅)-L-D-C(O)—(B′)ᵣ-A;
R₂₀ and R₄₃ are independently alkyl, substituted alkyl, aryl, substituted aryl, arylalkyl, substituted arylalkyl, heteroaryl, substituted heteroaryl, heteroarylalkyl, heteroalkyl or substituted heteroalkyl;
R₂₉ to R₃₄ and R₄₄ to R₄₇ are independently hydrogen, alkyl, or substituted alkyl;
a and b are independently 0, 1, 2, 3 or 4;
k and m are independently 1, 2, 3, 4 or 5;
h, n, o and p are independently 0, 1, 2, 3, 4 or 5;
q and q′ are independently 2, 3, 4 or 5;
e and t are independently 0, 1 or 2;
Q is —NR₂₉;
X is —NR₃₀ or —O—;
Y is —NR₃₁ or —O—;
Z is —NR₃₂ or —O—;
Q′ is —NR₃₃;
B′ is —NH—C(R₃₄)—C(O)— wherein R₃₄ is hydrogen, alkyl, substituted alkyl, aryl, substituted aryl, arylalkyl, substituted arylalkyl, heteroaryl, substituted heteroaryl, heteroarylalkyl, heteroalkyl or substituted heteroalkyl;
I is —C(R₅₆R₅₇)—, —S—, —O— or —Se—;
U is —C(R₅₈R₅₉)—, —S—, —O— or —Se—;
R₆₀ is hydrogen or alternatively R₆₀ and R₅₃ together with the atoms to which they are bonded form a cycloheteroalkyl or substituted cycloheteroalkyl ring;
R₆₁ is hydrogen or alternatively R₆₀ and R₅₂ together with the atoms to which they are bonded form a cycloheteroalkyl or substituted cycloheteroalkyl ring;
V is —NR₆₁, 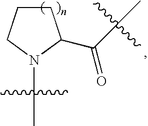
or —O—;
r is 0 or 1;
L and L′ are alkyl, substituted alkyl, heteroalkyl or substituted alkyl, aryl or substituted aryl;
A is OH, —NHCH₂CH₂SH, 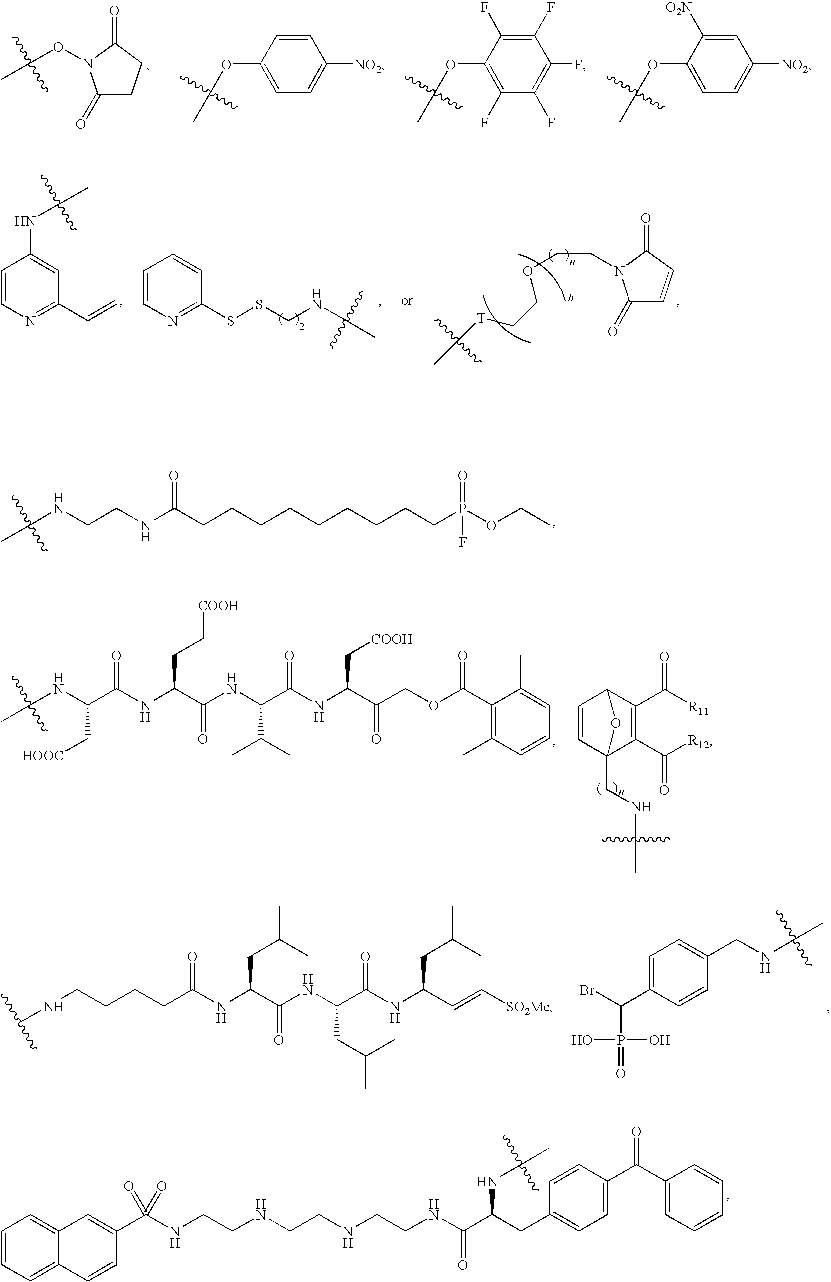 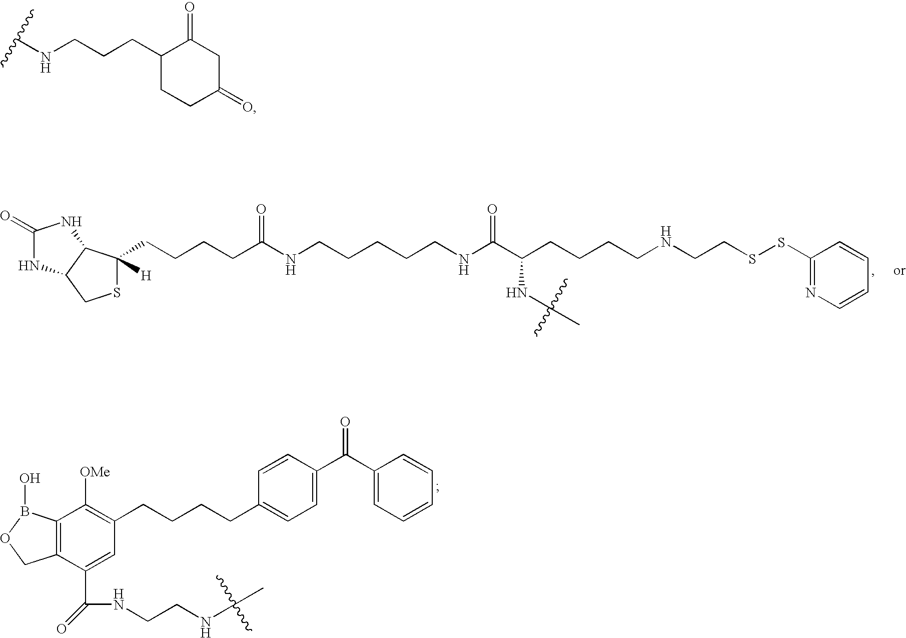
R₁₁ and R₁₂ are independently alkyl, substituted alkyl, acyl, substituted acyl, alkoxy, substituted alkoxy, aryl, substituted aryl, azido alkyl, alkynyl, substituted alkynyl, amino, or substituted amino;
T is —NR₃₄;
D is 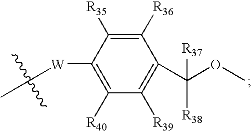
G is (CH₂)ₙ—(C(O))ₚ—N(R^{c})N(CH₂)qR^{c}, —(CH₂)ₙ—(C(O))—, 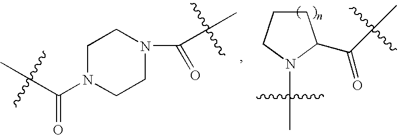
or NH₂;
R^{c} is H, alkyl or can be taken together with the nitrogen atoms to which they are bonded faun a cycloheteroalkyl or substituted cycloheteroalkyl ring;
R₃₇ and R₃₈ are independently hydrogen, alkyl or substituted alkyl;
R₃₅, R₃₆, R₃₉ and R₄₀ are independently hydrogen, nitro, alkyl, substituted alkyl, —NR₄₁R₄₂, —S(O)ₑR₄₃, aryloxy, substituted aryloxy, alkoxy or substituted alkoxy provided that at least one of R₃₅, R₃₆, R₃₇ and R₃₈ is nitro, aryloxy, substituted aryloxy, alkoxy or substituted alkoxy; and
W is —O—, —S— or —NR₄₇;
provided that the optical labeling molecule contains at least one zwitterionic pair.

2. The optical labeling molecule of claim 1 having structural formula (I), or a salt or solvate thereof: 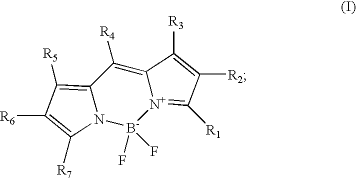
wherein the optical labeling molecule comprises a fluorophore with a derivative tail, and the derivative tail comprises at least two amide bonds; wherein
R₁ to R₇ are each independently hydrogen, acyl, substituted acyl, alkoxy, substituted alkoxy, alkoxycarbonyl, substituted alkoxycarbonyl, alkyl, substituted alkyl, amino, aryl, substituted aryl, arylalkyl, substituted arylalkyl, aryloxycarbonyl, substituted aryloxycarbonyl, carboxyl, cyano, heteroaryl, substituted heteroaryl, heteroarylalkyl, substituted heteroarylalkyl, heteroalkyl, substituted heteroalkyl, halo, nitro, —(CH₂)ₙN⁺(CH₃)₃, —S(O)ₜR₂₀, —SO₃H, —(CH₂)ₙS(O)ₙOH, —(CH₂)ₙS(O)₂O⁻, —OP(O)(O⁻)₂, —(CH₂)ₙOP(O)(O⁻)₂, 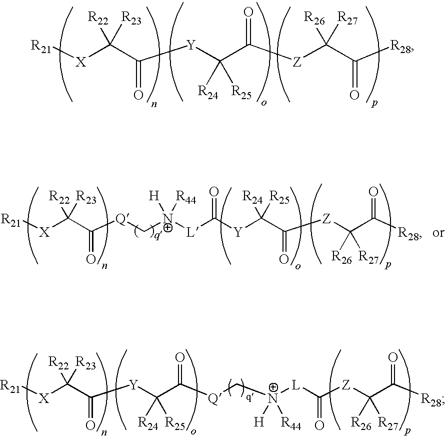
provided that at least one and only one of R₁ to R₇ is 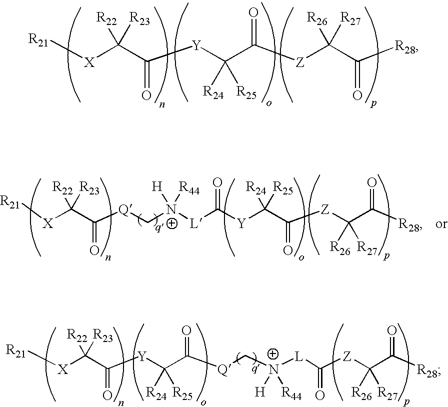
R₂₂, R₂₃, R₂₄, R₂₅, R₂₆ and R₂₇ are independently, hydrogen, alkyl, substituted alkyl, amino, aryl, substituted aryl, arylalkyl, substituted arylalkyl, heteroaryl, substituted heteroaryl, heteroarylalkyl, heteroalkyl or substituted heteroalkyl, nitro, —(CH₂)ₙN⁺(CH₃)₃, —S(O)ₜR₂₀, —SO₃H, —(CH₂)ₙS(O)OH, —(CH₂)ₙS(O)₂O⁻, —OP(O)(O⁻)₂, —(CH₂)ₙOP(O)(O⁻)₂;
R₂₁ is —(CH₂)ₘ—C(O)—, —(CH₂)ₘ—C(O)-Q′(CH₂)_{q}—N⁺H(R₄₆)-L′-C(O)—, 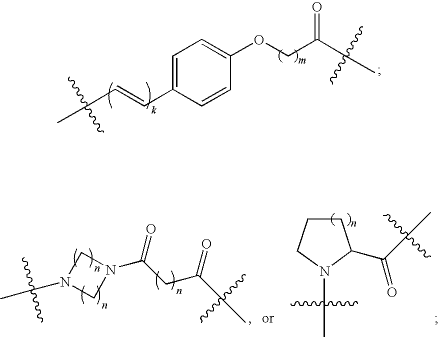
R₂₈ is -Q-L-C(O)-A; -Q(CH₂)_{q}—N⁺H(R₄₅)-L-C(O)-A, -Q-L-D-C(O)—(B′)ᵣ-A or -Q(CH₂)_{q}—N⁺H(R₄₅)-L-D-C(O)—(B′)ᵣ-A;
R₂₀ and R₄₃ are independently alkyl, substituted alkyl, aryl, substituted aryl, arylalkyl, substituted arylalkyl, heteroaryl, substituted heteroaryl, heteroarylalkyl, heteroalkyl or substituted heteroalkyl;
R₂₉ to R₃₄ and R₄₄ to R₄₇ are independently hydrogen, alkyl or substituted alkyl;
k and m are independently 1, 2, 3, 4 or 5;
n, o and p are independently 0, 1, 2, 3, 4 or 5;
q and q′ are independently 2, 3, 4 or 5;
e and t are independently 0, 1 or 2;
Q is —NR₂₉;
X is —NR₃₀ or —O—;
Y is —NR₃₁ or —O—;
Z is —NR₃₂ or —O—;
Q′ is —NR₃₃;
B′ is —NH—C(R₃₄)—C(O)— wherein R₃₄ is hydrogen, alkyl, substituted alkyl, aryl, substituted aryl, arylalkyl, substituted arylalkyl, heteroaryl, substituted heteroaryl, heteroarylalkyl, heteroalkyl or substituted heteroalkyl;
r is 0 or 1;
L and L′ are alkyl, substituted alkyl, heteroalkyl or substituted alkyl, aryl or substituted aryl;
A is OH, —NHCH₂CH₂SH, 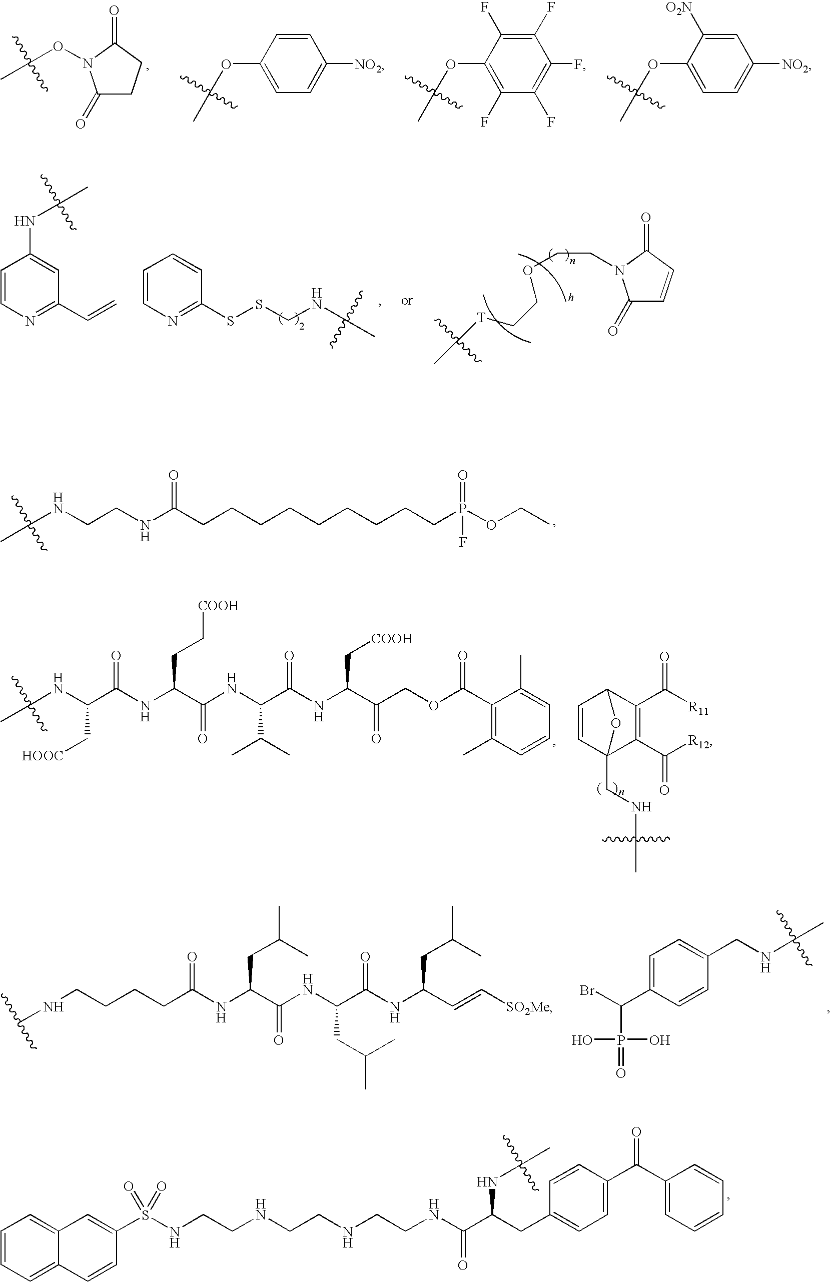 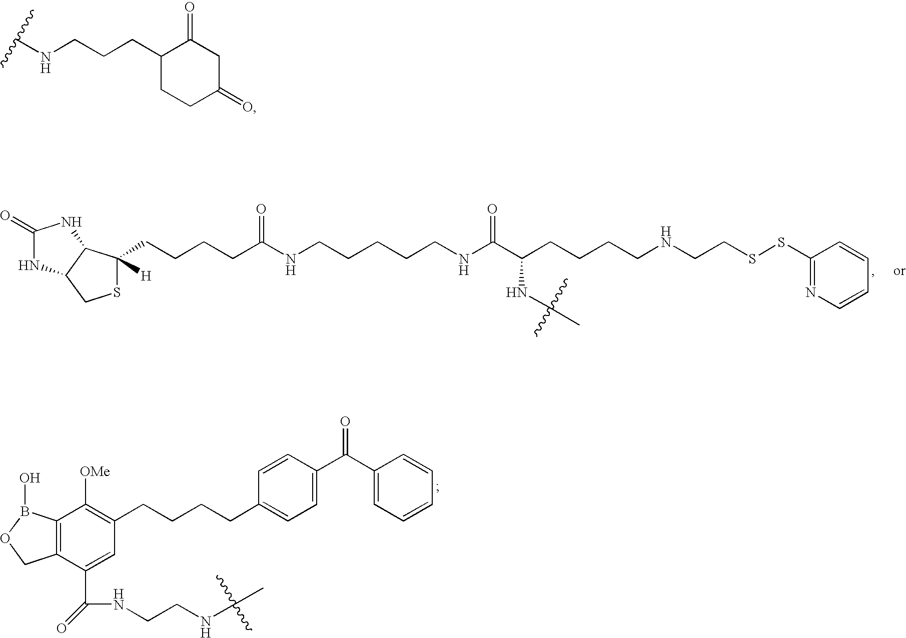
R₁₁ and R₁₂ are independently alkyl, substituted alkyl, acyl, substituted acyl, alkoxy, substituted alkoxy, aryl, substituted aryl, azido alkyl, alkynyl, substituted alkynyl, amino, or substituted amino;
T is —NR₃₄;
D is 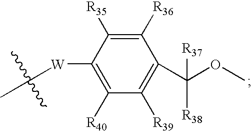
R₃₇ and R₃₈ are independently hydrogen, alkyl or substituted alkyl;
R₃₅, R₃₆, R₃₉ and R₄₀ are independently hydrogen, nitro, alkyl, substituted alkyl, —NR₄₁R₄₂, —S(O)ₙR₄₃, aryloxy, substituted aryloxy, alkoxy or substituted alkoxy provided that at least one of R₃₅, R₃₆, R₃₇ and R₃₈ is nitro, aryloxy, substituted aryloxy, alkoxy or substituted alkoxy; and
W is —O—, —S— or —NR₄₇;
provided that R₁ to R₇ contains at least one zwitterionic pair.

3. The optical labeling molecule of claim 2, wherein R₁ to R₇ are each independently hydrogen, alkyl, substituted alkyl, aryl, substituted aryl, heteroaryl, substituted heteroaryl or

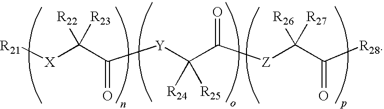

4. The optical labeling molecule of claim 2, wherein R₂, R₃, R₄, R₅, R₆ and R₇ are independently hydrogen, alkyl, substituted alkyl, aryl, substituted aryl, heteroaryl or substituted heteroaryl; and R₁ is

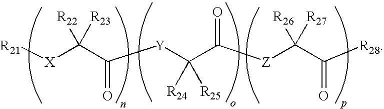

5. The optical labeling molecule of claim 2, wherein R₃ is alkyl or substituted alkyl; R₅ is alkyl or substituted alkyl; R₇ is aryl or substituted aryl or heteroaryl or substituted heteroaryl; R₂, R₄ and R₆ are hydrogen; and R₁ is

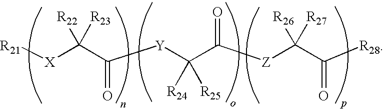

6. The optical labeling molecule of claim 2, wherein R₃, R₅, and R₇ are each independently alkyl or substituted alkyl; R₂, R₄, and R₆ are hydrogen; and R₁ is

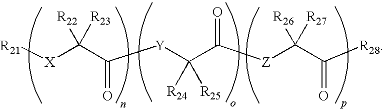

7. The optical labeling molecule of claim 2, wherein R₃, R₅, and R₇ are each independently alkyl or substituted alkyl; R₄ is aryl, substituted aryl, heteroaryl, or substituted heteroaryl; R₂ and R₆ are hydrogen; and R₁ is

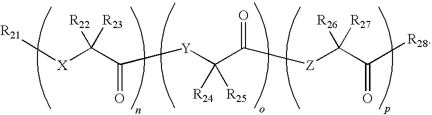

8. The optical labeling molecule of claim 2, wherein R₁, R₅, and R₇ are independently alkyl or substituted alkyl; R₂, R₄, and R₆ are hydrogen and R₃ is

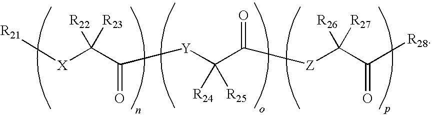

9. The optical labeling molecule of claim 2, wherein R₁ and R₅ are each independently alkyl or substituted alkyl; R₇ is aryl, substituted aryl, heteroaryl or substituted heteroaryl; R₂, R₄, and R₆ are hydrogen; and R₃ is

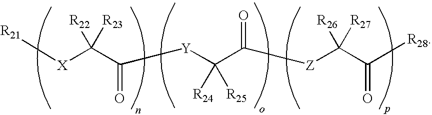

10. The optical labeling molecule of claim 2, wherein R₁ and R₇ are each independently alkyl, substituted alkyl, aryl or substituted aryl, heteroaryl or substituted heteroaryl; R₅ is alkyl or substituted alkyl; R₂ and R₆ are hydrogen; R₄ is hydrogen, alkyl, substituted alkyl, aryl or substituted aryl, heteroaryl or substituted heteroaryl; and R₃ is

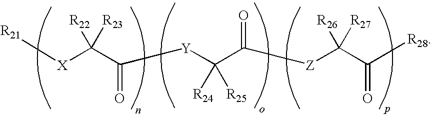

11. The optical labeling molecule of claim 2, wherein R₃ is methyl, propyl or —(CH₂)₄N⁺(CH₃)₃.

12. The optical labeling molecule of claim 2, wherein R₅ is methyl, propyl or —(CH₂)₄N⁺(CH₃)₃.

13. The optical labeling molecule of claim 2, wherein R₇ is phenyl, p-methoxyphenyl, thiophenyl, methyl, propyl, butyl, heptyl, or —(CH₂)₄N⁺(CH₃)₃.

14. The optical labeling molecule of claim 2, wherein R₃ is methyl, propyl or —(CH₂)₄N⁺(CH₃)₃; R₅ is methyl, propyl, or —(CH₂)₄N⁺(CH₃)₃; R₇ is phenyl, p-methoxyphenyl, thiophenyl, methyl, propyl, butyl, heptyl or —(CH₂)₄N⁺(CH₃)₃; R₂, R₄ and R₆ are hydrogen; and R₁ is

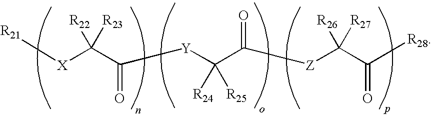

15. The optical labeling molecule of claim 2, wherein R₃ is methyl; R₅ is —(CH₂)₄N⁺(CH₃)₃; R₇ is phenyl, p-methoxyphenyl, thiophenyl, methyl, propyl, butyl, heptyl, or —(CH₂)₄N⁺(CH₃)₃; R₂, R₄ and R₆ are hydrogen; and R₁ is

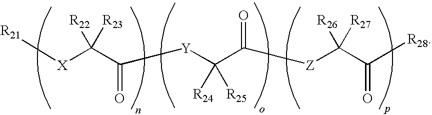

16. The optical labeling molecule of claim 2, wherein R₃ is —(CH₂)₄N⁺(CH₃)₃; R₅ is —(CH₂)₄N⁺(CH₃)₃, R₇ is phenyl, p-methoxyphenyl, methyl, propyl, butyl, heptyl, or —(CH₂)₄N⁺(CH₃)₃; R₂, R₄ and R₆ are hydrogen; and R₁ is

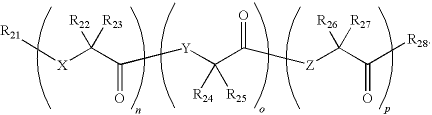

17. The optical labeling molecule of claim 2, wherein R₃ is propyl; R₅ is propyl; R₇ is —(CH₂)₄N⁺(CH₃)₃; R₂, R₄ and R₆ are hydrogen; and R₁ is

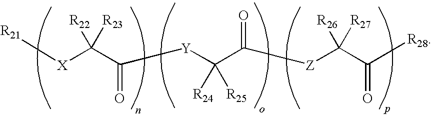

18. The optical labeling molecule of claims 2, wherein R₂₁ is —(CH₂)ₘ—C(O)—; n is 1; X is —NH—; o is 0; p is 1; Z is —NH—; and R₂₈ is -Q-L-C(O)-A.

19. The optical labeling molecule of claim 2, wherein R₂₁ is —(CH₂)₂—C(O)—, n is 1, X is —NH—, R₂₂ is hydrogen, R₂₃ is —CH₂SO₃⁻, CH₂OP(O)(O⁻)₂ or —(CH₂)ₙN⁺(CH₃)₃, o is 0, p is 1, Z is —NH—, R₂₆ and R₂₇ are hydrogen, R₂₈ is -Q-L-C(O)-A, and Q is —NH—, and L is —(CH₂)₄—.

20. The optical labeling molecule of claim 2, R₂₁ is —(CH₂)ₘ—C(O)—, n is 1, X is —NH—, o is 0, p is 0, and R₂₈ is -Q(CH₂)_{q}—N⁺H(R₂₁)-L-D-C(O)—(B′)ᵣ-A.

21. The optical labeling molecule of claim 2, wherein R₂₁ is —(CH₂)₂—C(O)—, n is 1, X is —NH—, R₂₂ is hydrogen, R₂₃ is —CH₂SO₃— or —(CH₂)ₙN⁺(CH₃)₃, o is 0, p is 0, R₂₈ is -Q(CH₂)₂—N⁺H(R₄₅)-L-D-C(O)-A, Q is —NH—, L is —(CH₂)₂—, and R₄₅ is methyl.

22. The optical labeling molecule of claim 21, wherein D is

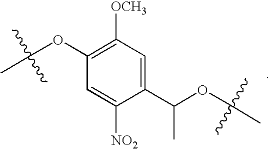

23. The optical labeling molecule of claim 22, wherein r is 1; and R₄₅ is hydrogen.

24. The optical labeling molecule of claim 2, wherein R₂₁ is —(CH₂)ₘ—C(O)—, n is 1, X is —NH—, o is 1, Y is —NH—, and R₂₈ is -Q-L-C(O)-A.

25. The optical labeling molecule of claim 2, wherein R₂₁ is —(CH₂)₂—C(O)—, R₂₂ is hydrogen, R₂₃ is —CH₂SO₃— or —(CH₂)ₙN⁺(CH₃)₃, R₂₄ is hydrogen, R₂₅ is —CH₂SO₃— or —(CH₂)ₙN⁺(CH₃)₃, n is 1, X is —NH—, o is 1, Y is —NH—, R₂₈ is -Q-L-C(O)-A, Q is —NH—, L is —(CH₂)₄—.

26. The optical labeling molecule of claim 2, wherein R₂₁ is —(CH₂)ₘ—C(O)—, n is 1, X is —NH—, o is 1, Y is —NH—, p is 0 and R₂₈ is -Q(CH₂)_{q}—N⁺H(R₄₅)-L-D-C(O)—(B′)ᵣ-A.

27. The optical labeling molecule of claim 2, wherein R₂₁ is —(CH₂)₂—C(O)—, n is 1, X is —NH—, o is 1, Y is —NH—, R₂₂ is hydrogen, R₂₃ is —CH₂SO₃— or —(CH₂)ₙN⁺(CH₃)₃, R₂₄ is hydrogen, R₂₅ is —CH₂SO₃— or —(CH₂)ₙN⁺(CH₃)₃, p is 0, R₂₈ is -Q(CH₂)₂—N⁺H(R₄₅)-L-D-C(O)-A, Q is —NH—, L is —(CH₂)₂—, and R₄₅ is methyl.

28. The compound of claim 27, wherein D is

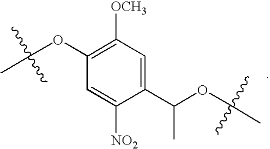

29. The compound of claim 28, wherein r is 1; and R₄₅ is hydrogen.

30. The optical labeling molecule of claim 1 having structural Formula (XV) or structural formula (XV′), or a salt or solvate thereof: 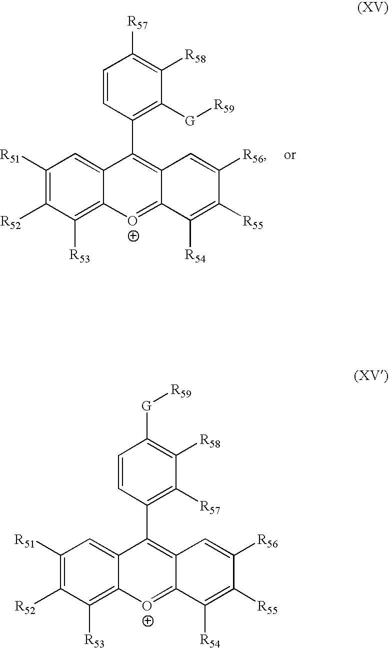
wherein:
R₅₁ to R₅₉ are each independently hydrogen, acyl, substituted acyl, alkoxy, substituted alkoxy, alkoxycarbonyl, substituted alkoxycarbonyl, alkyl, substituted alkyl, amino, substituted amino, aryl, substituted aryl, arylalkyl, substituted arylalkyl, aryloxycarbonyl, substituted aryloxycarbonyl, carboxyl, cyano, heteroaryl, substituted heteroaryl, heteroaryl alkyl, substituted heteroarylalkyl, heteroalkyl, substituted heteroalkyl, halo, nitro, —(CH₂)ₙN⁺(CH₃)₃, —S(O)ₜR²⁰, —SO₃H, —(CH₂)ₙS(O)ₙOH, —(CH₂)ₙS(O)₂O⁻, —OP(O)(O⁻)₂, —(CH₂)OP(O)(O⁻)₂, 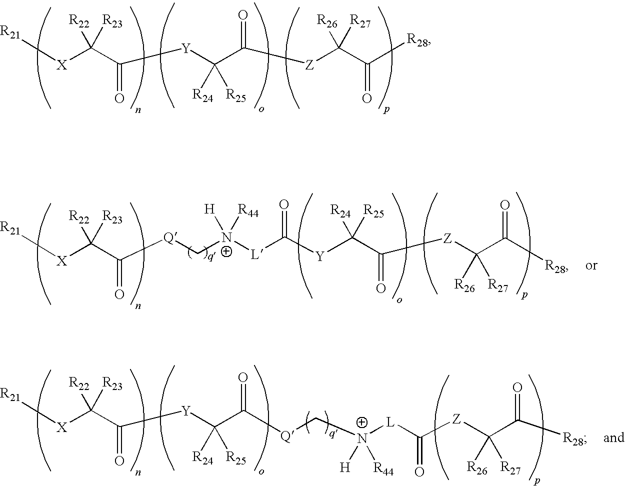
G is (CH₂)ₙ—(C(O))ₚ—N(R^{c})N(CH₂)qR^{c}, —(CH₂)ₙ—(C(O))—,

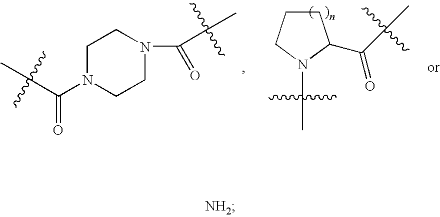

31. The optical labeling molecule of claim 30, wherein R₅₇ or R₅₉ is acyl, alkyl, substituted alkyl, alkoxy, heteroalkyl, substituted heteroalkyl, halo, nitro, —S(O)ₜR₂₀, —SO₃H, or 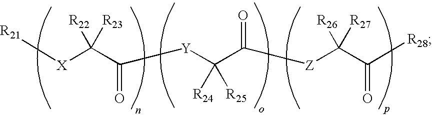
and R₅₁ to R₅₆ are each independently acyl, substituted acyl, alkyl, substituted alkyl, amino, substituted amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃, or

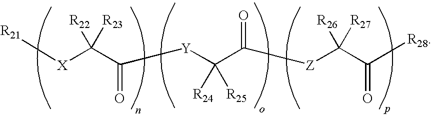

32. The optical labeling molecule of claim 30, wherein R₅₇ or R₅₉ is acyl, alkyl, substituted alkyl, alkoxy, heteroalkyl, substituted heteroalkyl, halo, nitro, —S(O)ₜR₂₀, or —SO₃H; and R₅₂ and R₅₅ are each independently acyl, substituted acyl, alkyl, substituted alkyl, amino, substituted amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃, or

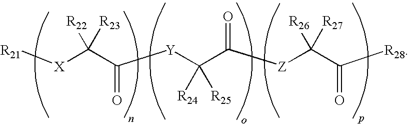

33. The optical labeling molecule of claim 30, wherein R₅₇ or R₅₈ are acyl, alkyl or substituted alkyl; R₅₉ is 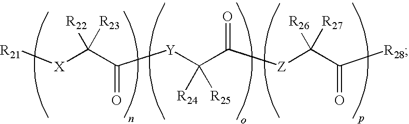
and R₅₂ and R₅₅ are each independently amino or substituted amino.

34. The optical labeling molecule of claim 33, wherein G is

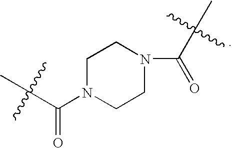

35. The optical labeling molecule of claim 33, wherein G is

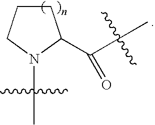

36. The optical labeling molecule of claim 1 having structural Formula (III), or a salt or solvate thereof: 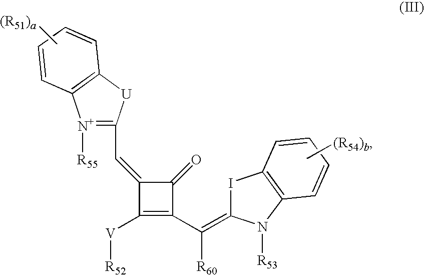
wherein:
a and b are independently 0, 1, 2, 3 or 4;
R₅₅ is independently hydrogen, acyl, substituted acyl, alkoxycarbonyl, substituted alkoxycarbonyl, alkyl, substituted alkyl, aryl, substituted aryl, arylalkyl, substituted arylalkyl, aryloxycarbonyl, substituted aryloxycarbonyl, heteroaryl, substituted heteroaryl, heteroarylalkyl, substituted heteroarylalkyl, heteroalkyl, substituted heteroalkyl, —S(O)ₜR₄₃, 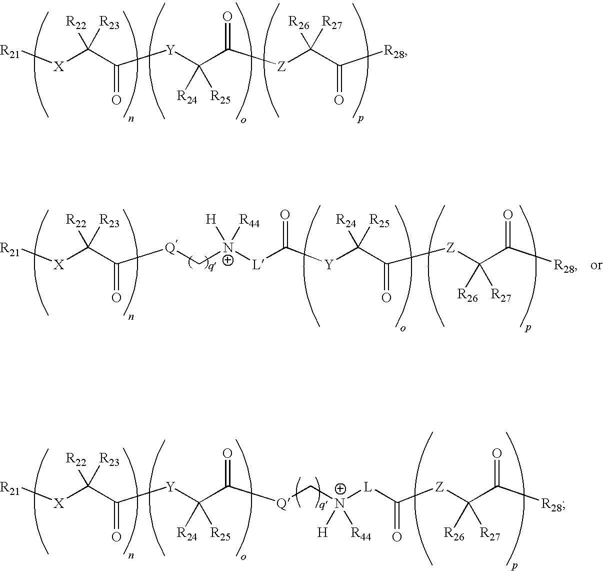
I is —C(R₅₆R₅₇)—, —S—, —O— or —Se—;
U is —C(R₅₈R₅₉)—, —S—, —O— or —Se—;
R₅₆, R₅₇, R₅₈ and R₅₉ are independently hydrogen or alkyl;
R₆₀ is hydrogen, or alternatively R₆₀ and R₅₃, together with the atoms to which they are bonded, form a cycloheteroalkyl or substituted cycloheteroalkyl ring;
V is —NR₆₁, 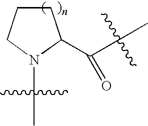
or —O—;
R₆₁ is hydrogen, alkyl, substituted alkyl, or alternatively R₆₁ and R₅₂, along with the atoms to which they are bonded, form a cycloheteroalkyl or substituted cycloheteroalkyl ring; and
R₂₀ to R₂₉, R₄₃, R₄₄, n, o, p, q′, t, e, Q′, X, Y, Z, L′, R₅₁ to R₅₄ and b are the same as defined in claim 1;
provided that:
(a) one and only one of R₅₁, R₅₂, R₅₃, R₅₄ or R₅₅ is 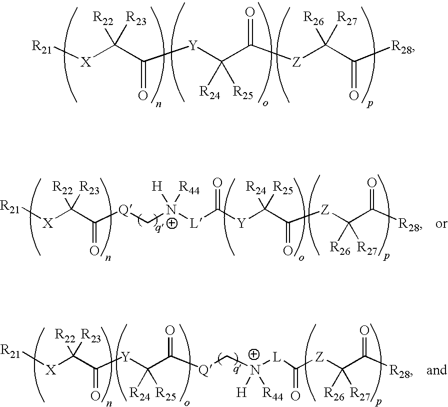
(b) R₅₁ to R₅₅ and R₆₀ contains at least one zwitterionic pair.

37. The optical labeling molecule of claim 36, wherein R₅₁ and R₅₄ are each independently hydrogen, acyl, alkyl, substituted alkyl, alkoxy, heteroalkyl, substituted heteroalkyl, halo, nitro, —S(O)ₜR₂₀, or —SO₃H; and R₅₂, R₅₃ and R₅₅ are each independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, —S(O)ₜR₄₃, or

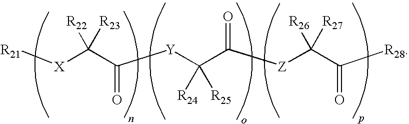

38. The optical labeling molecule of claim 36, wherein R₅₁ and R₅₄ are each independently hydrogen, acyl, alkyl, substituted alkyl, alkoxy, heteroalkyl, substituted heteroalkyl, halo, nitro, —S(O)ₜR₂₀, or —SO₃H; and R₅₃ or R₅₅ are each independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, or —S(O)ₜR₄₃; and R₅₂ is

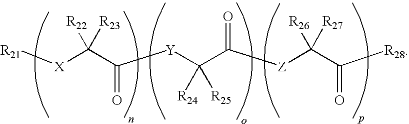

39. The optical labeling molecule of claim 36, wherein V is —NH—; R₆₁ and R₅₂ form a cycloheteroalkyl ring; I is —(CR₅₆R₅₇)— or S and U is —C(R₅₈R₅₉)—.

40. The optical labeling molecule of claim 36, wherein V is 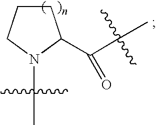
I is —(CR₅₆R₅₇)— or S and U is —C(R₅₈R₅₉)—.

41. The optical labeling molecule of claim 39, wherein R₅₆, R₅₇, R₅₈ and R₅₉ are —CH₃.

42. The optical labeling molecule of claim 36, wherein R₅₁ and R₅₄ are each independently hydrogen, acyl, alkyl, substituted alkyl, alkoxy, heteroalkyl, substituted heteroalkyl, halo, nitro, —S(O)ₜR₂₀, or —SO₃H; and R₅₃ and R₅₅ are each independently hydrogen, acyl, substituted acyl, alkyl, substituted alkyl, amino, aryl, substituted aryl, arylalkyl, substituted arylalkyl, heteroaryl, substituted heteroaryl, heteroarylalkyl, substituted heteroarylalkyl, heteroalkyl, substituted heteroalkyl, or —S(O)ₜR₄₃; R₅₂ is 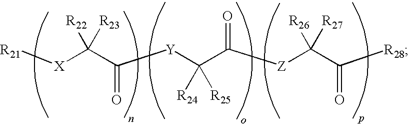
V is 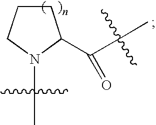
I is —(CR₅₅R₅₆)—; U is —(CR₅₈R₅₉)—; and R₅₆, R₅₇, R₅₈ and R₅₉ are —CH₃.

43. The optical labeling molecule of claim 1 having a structural Formula (XXI), or a salt or solvate thereof, 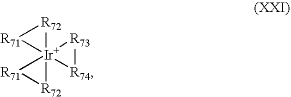
wherein:
R₇₁ is aryl or substituted aryl;
R₇₂ to R₇₄ are each independently heteroaryl or substituted heteroaryl; and
R₇₃ or R₇₄ contains at least one amino, substituted amino, acyl, substituted acyl,

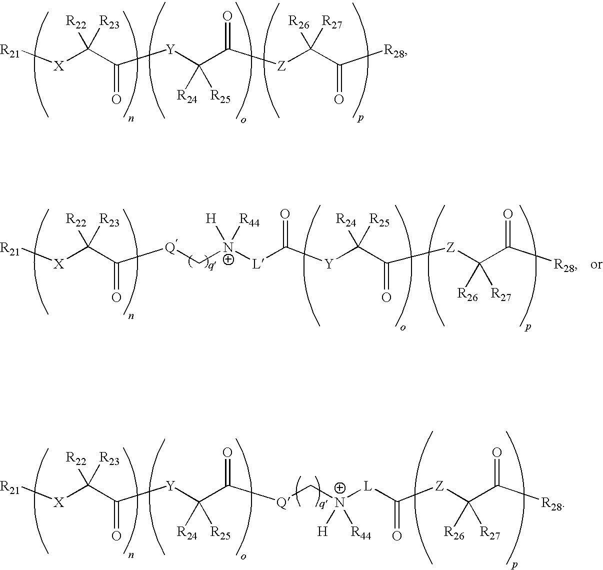

44. The optical labeling molecule of structural formula (I), (III), (XV), (XV′), or (XXI), which is selected from the group consisting of:

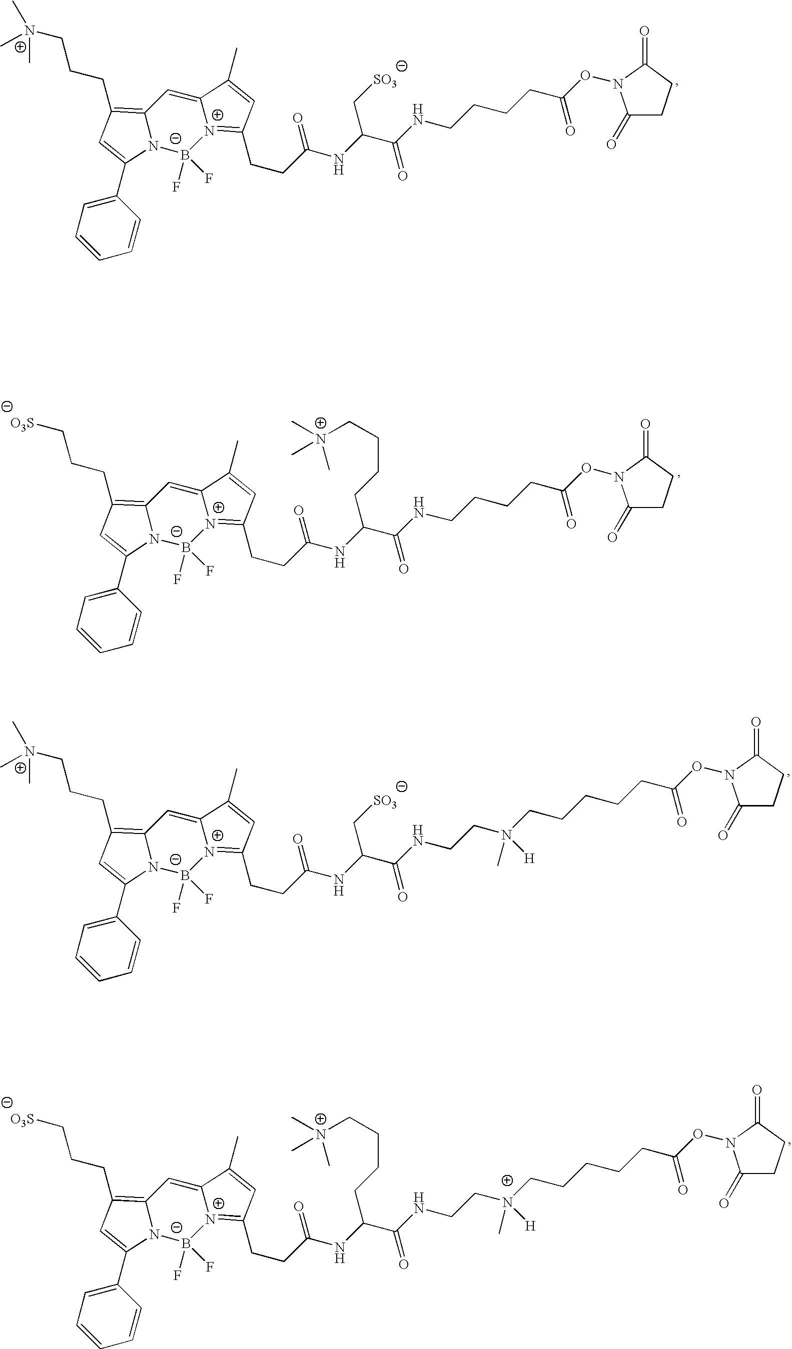

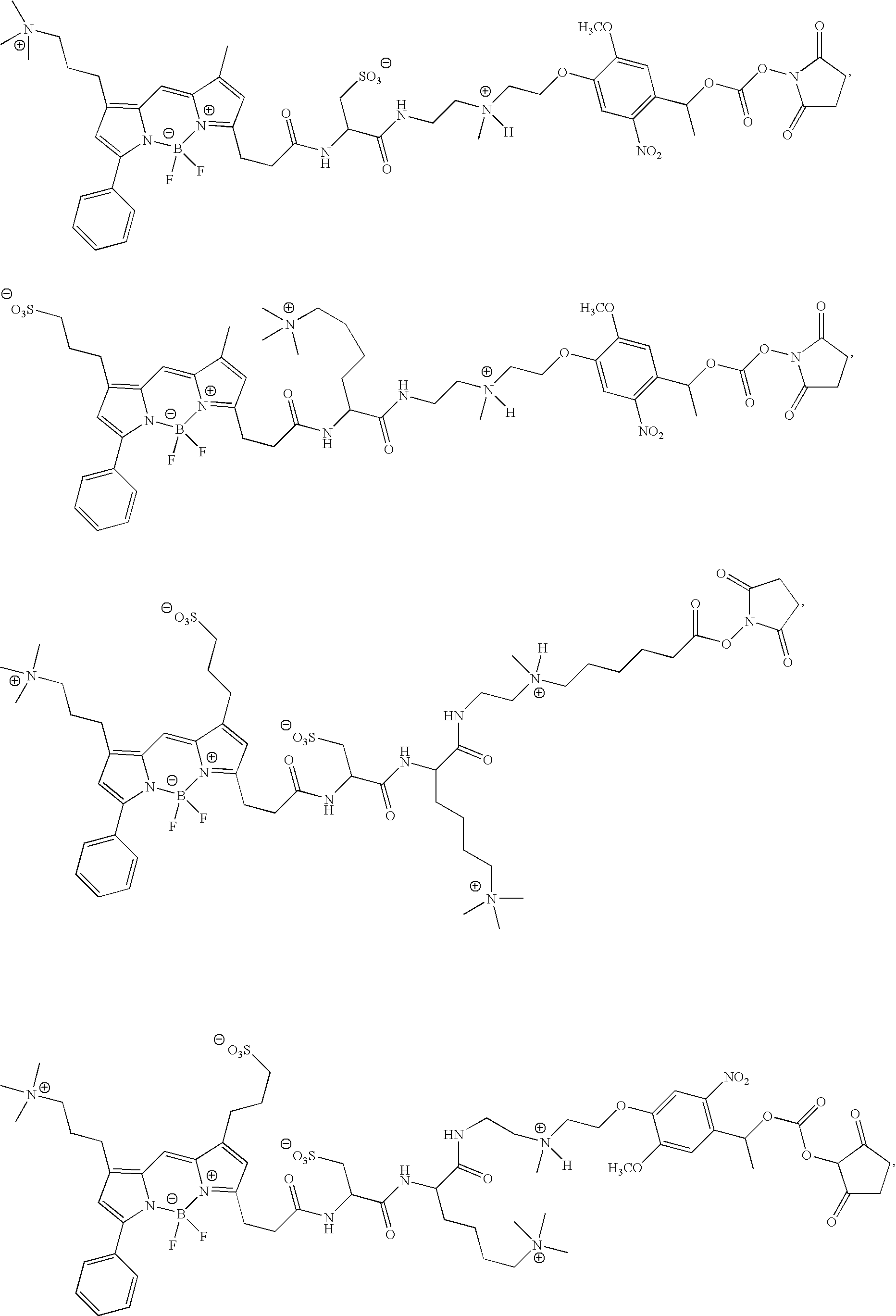

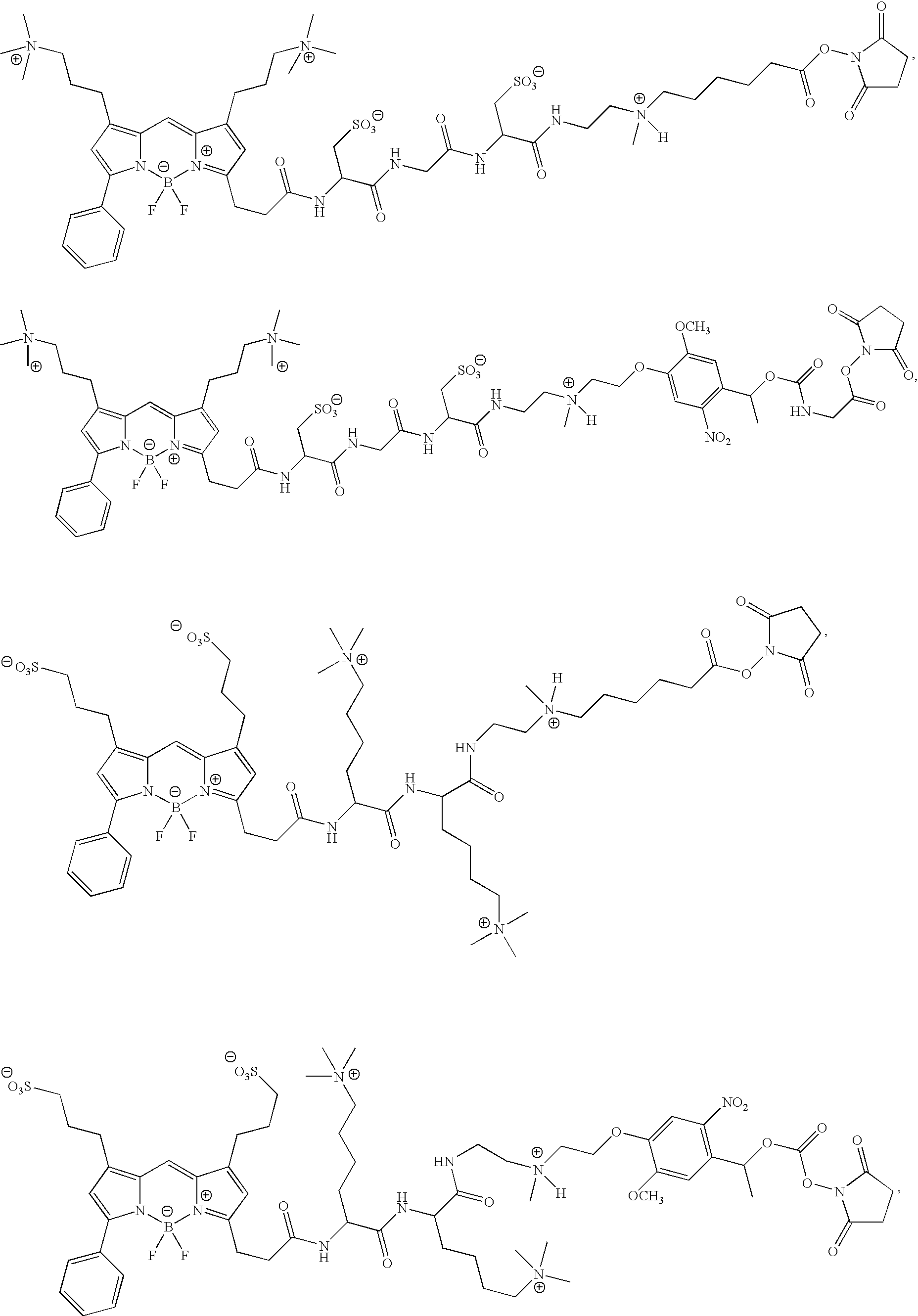

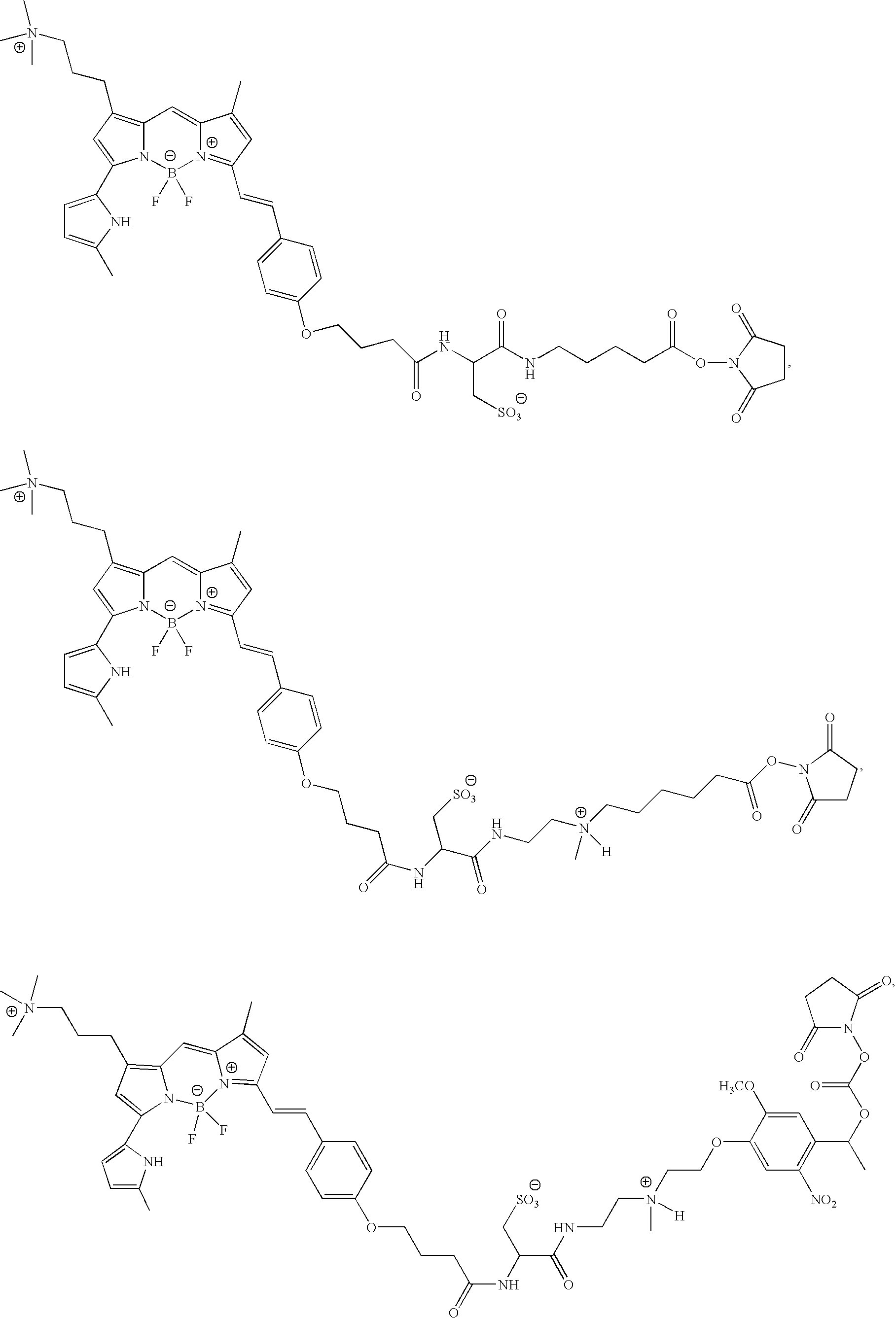

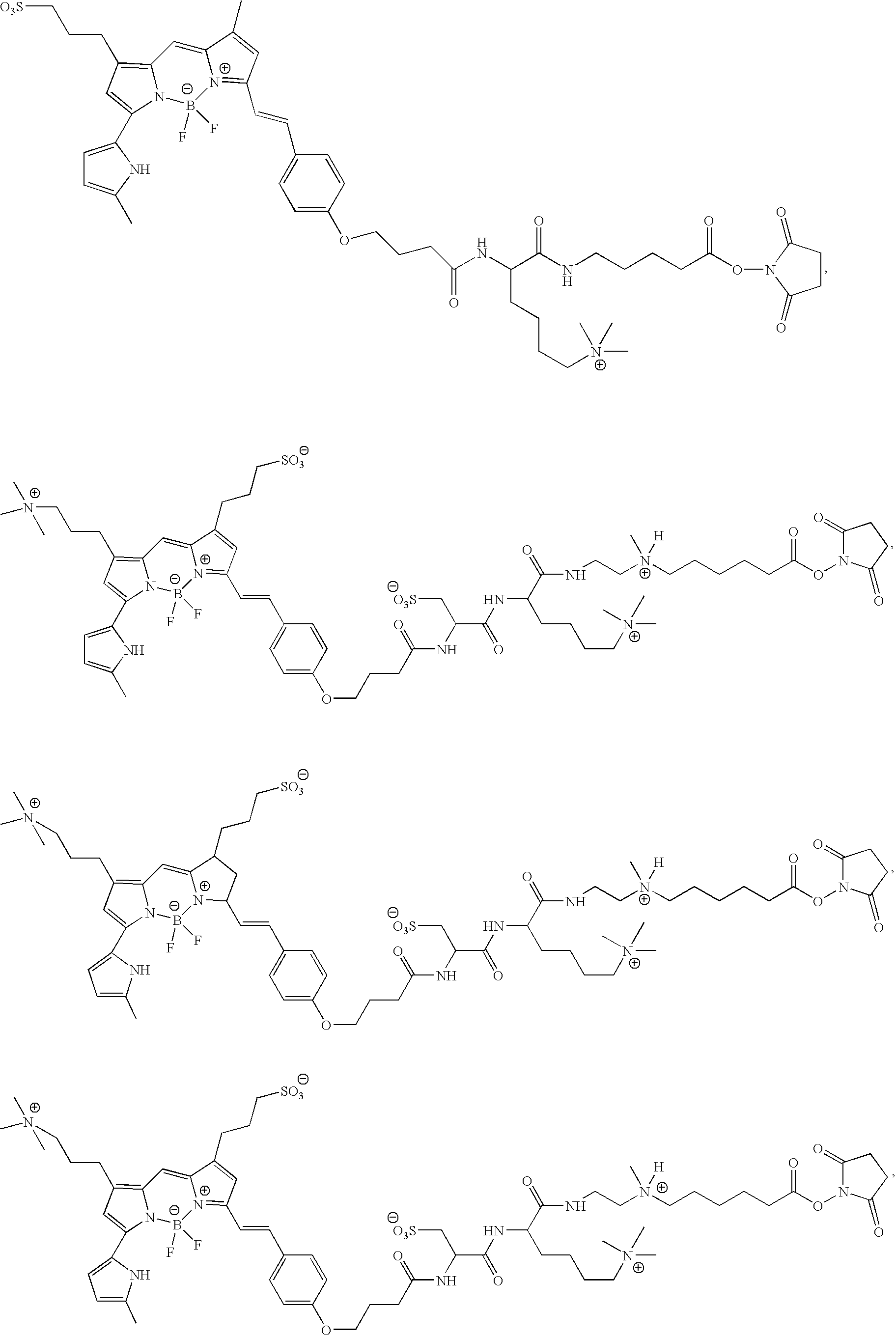

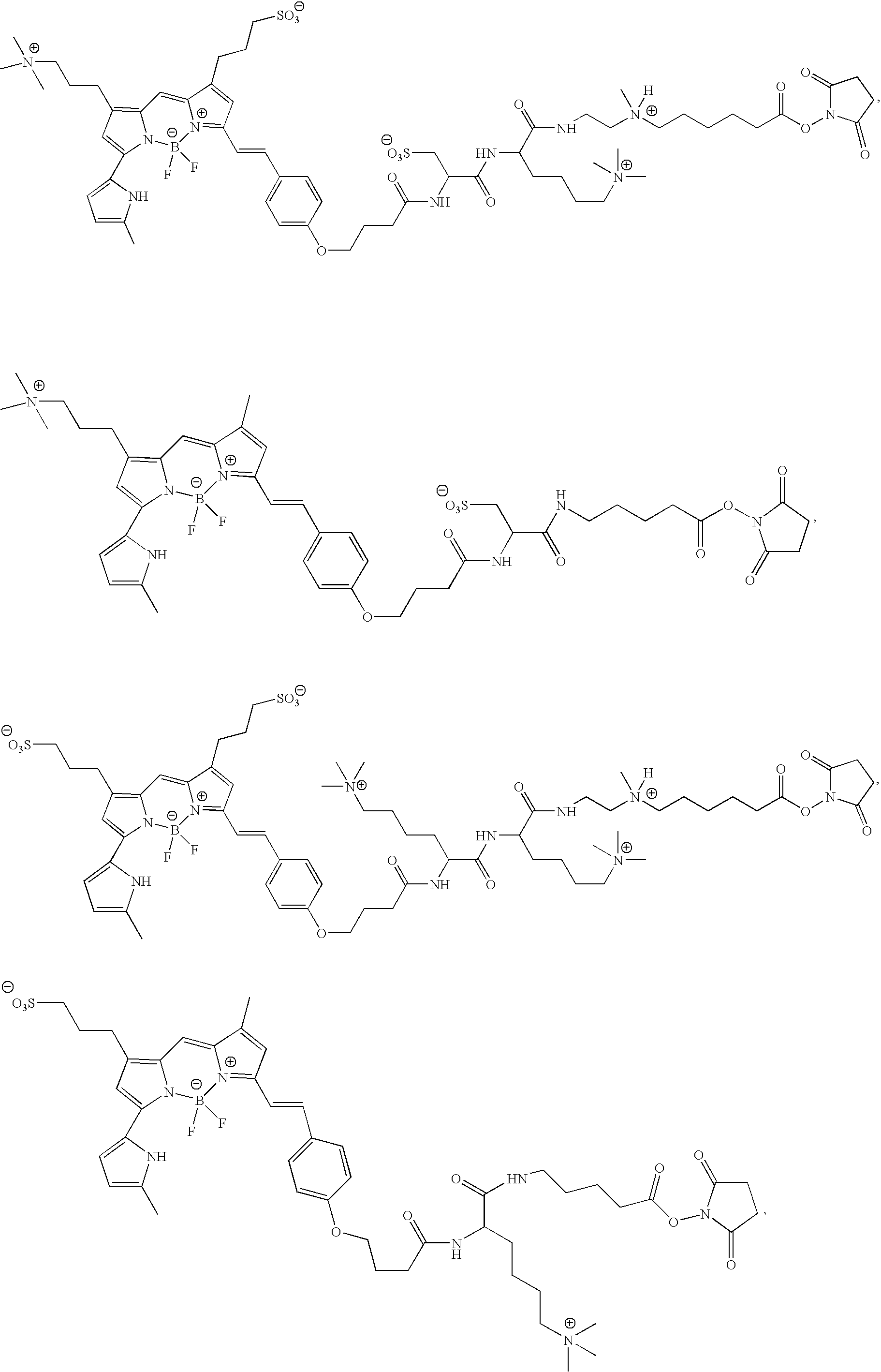

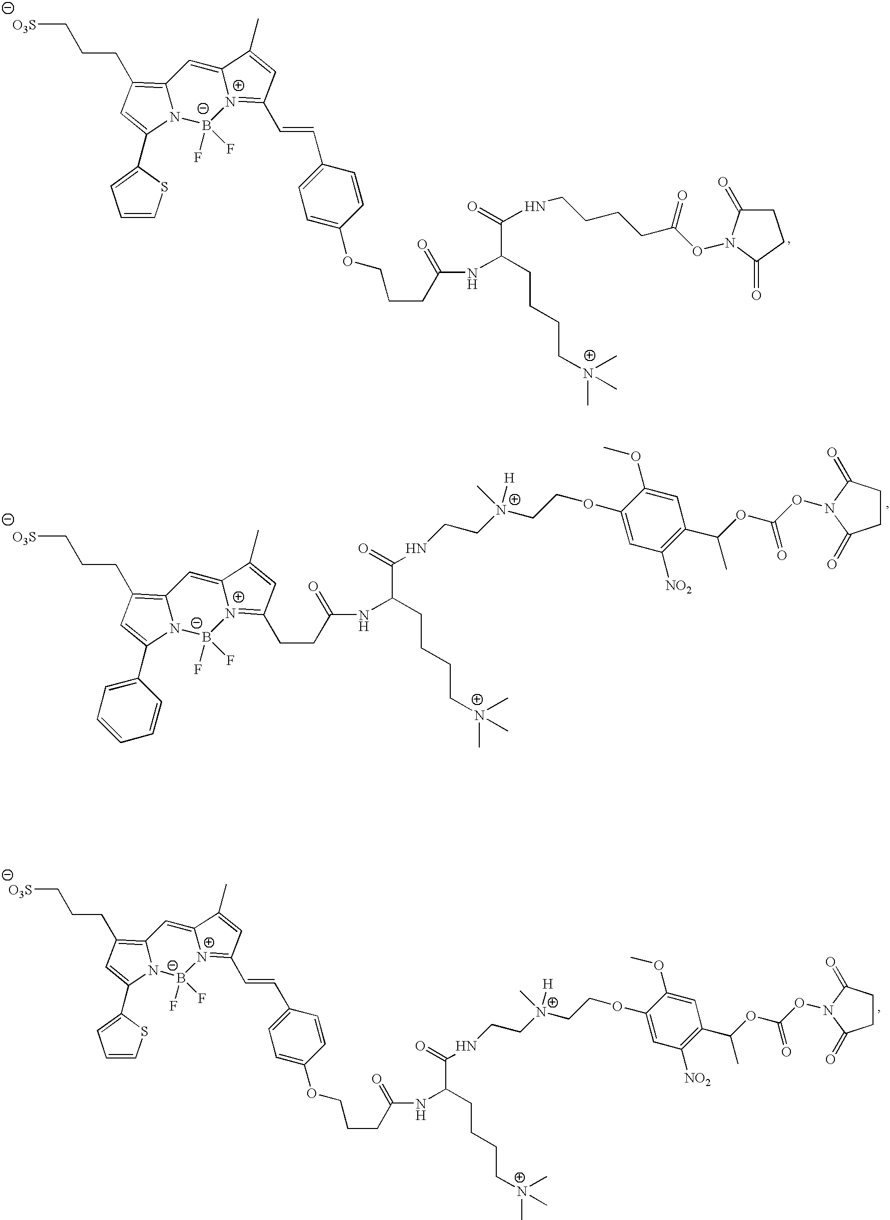

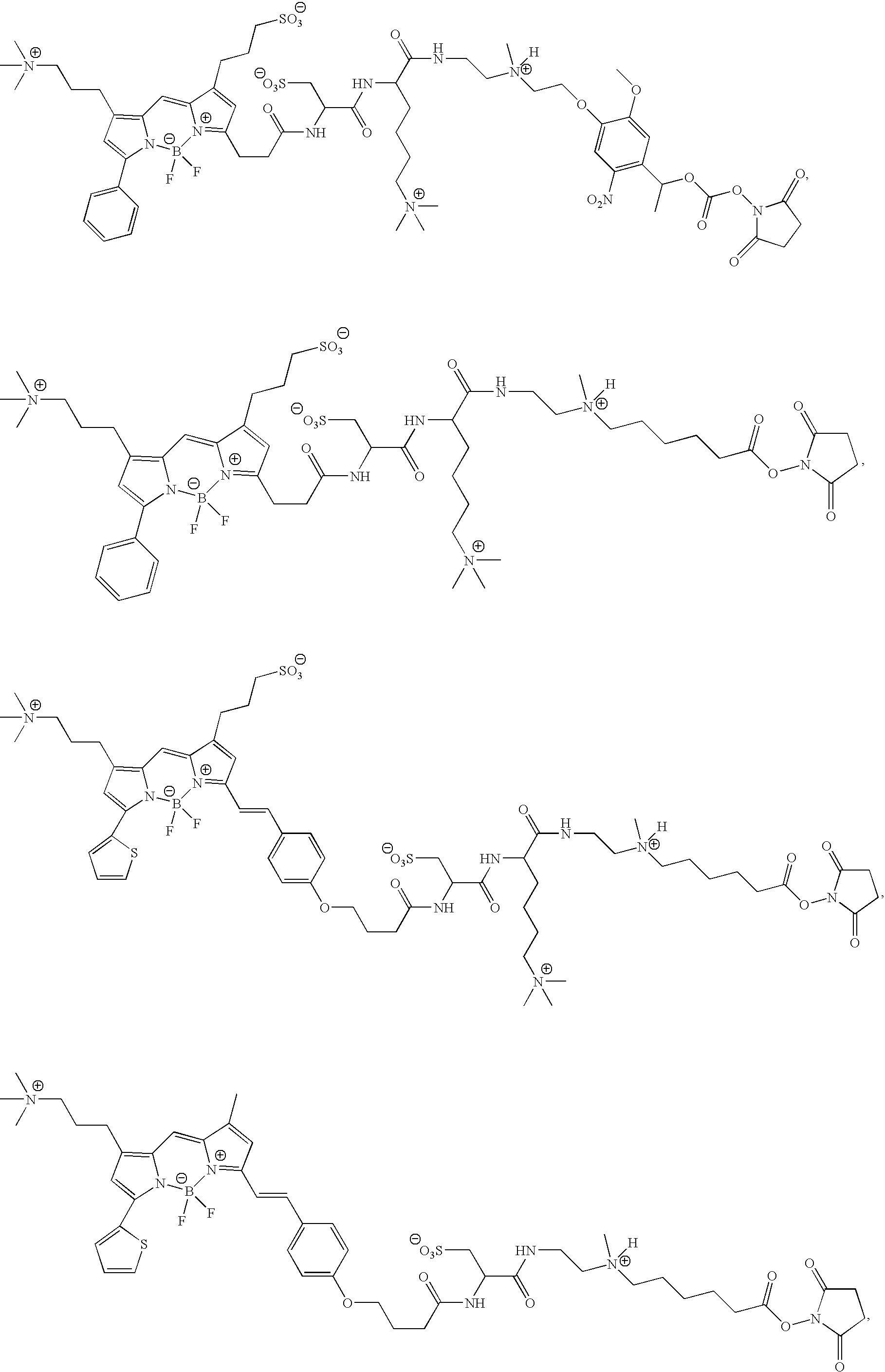

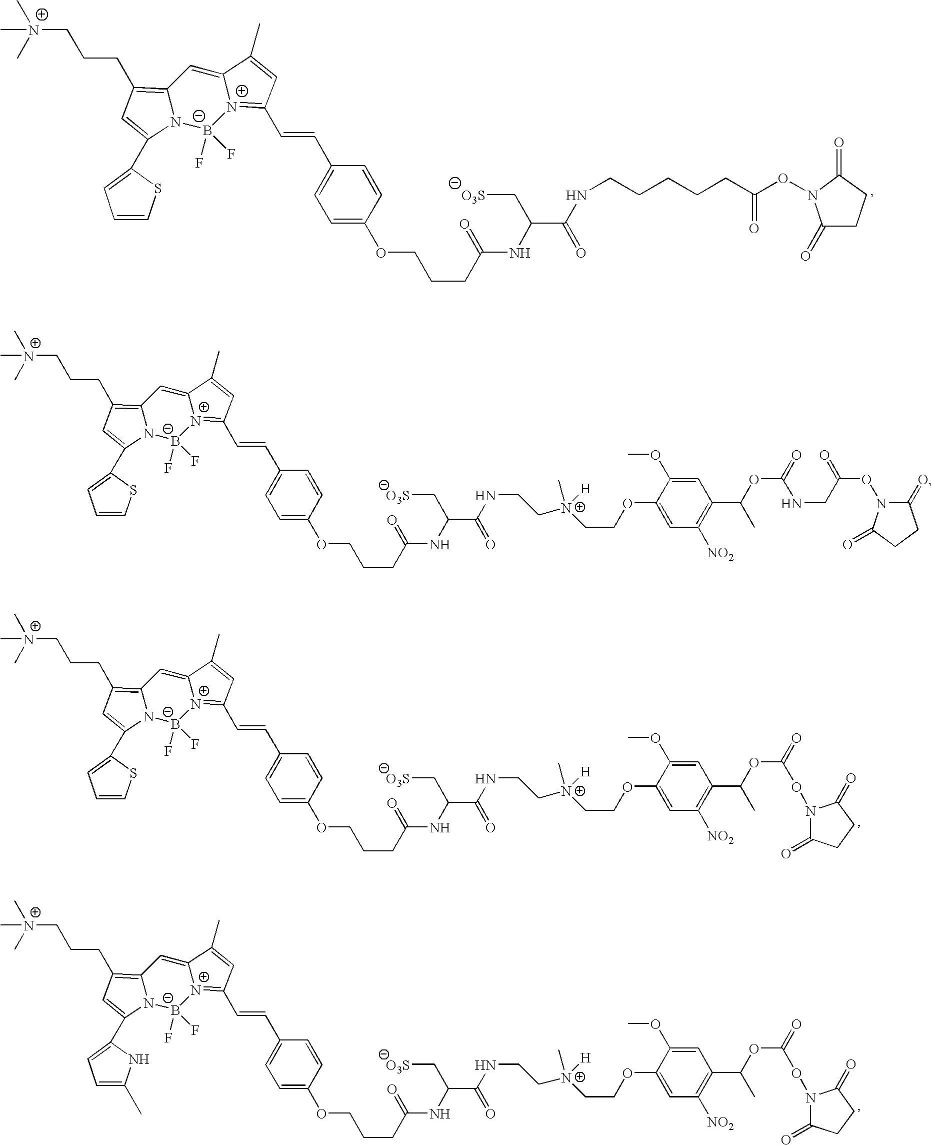

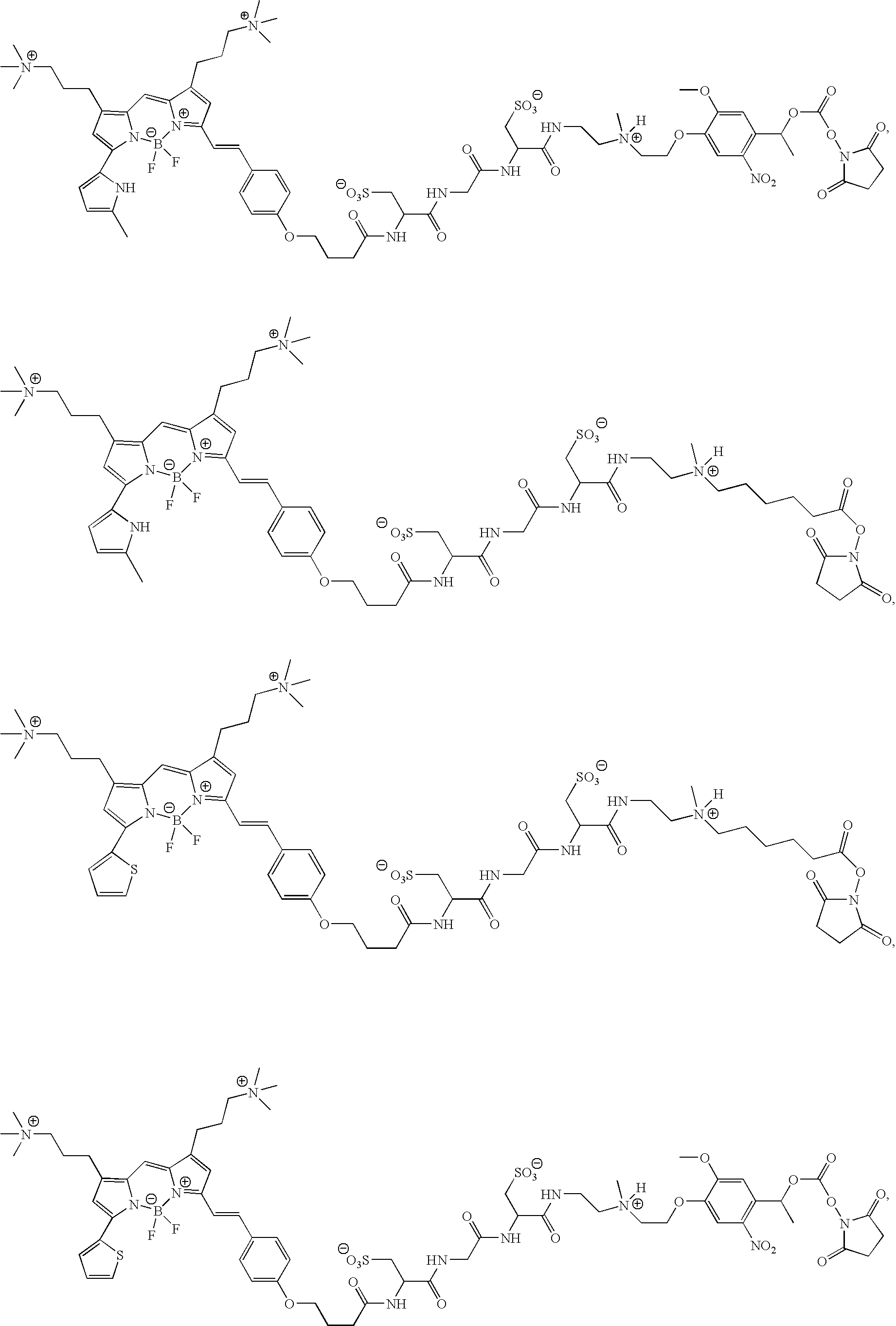

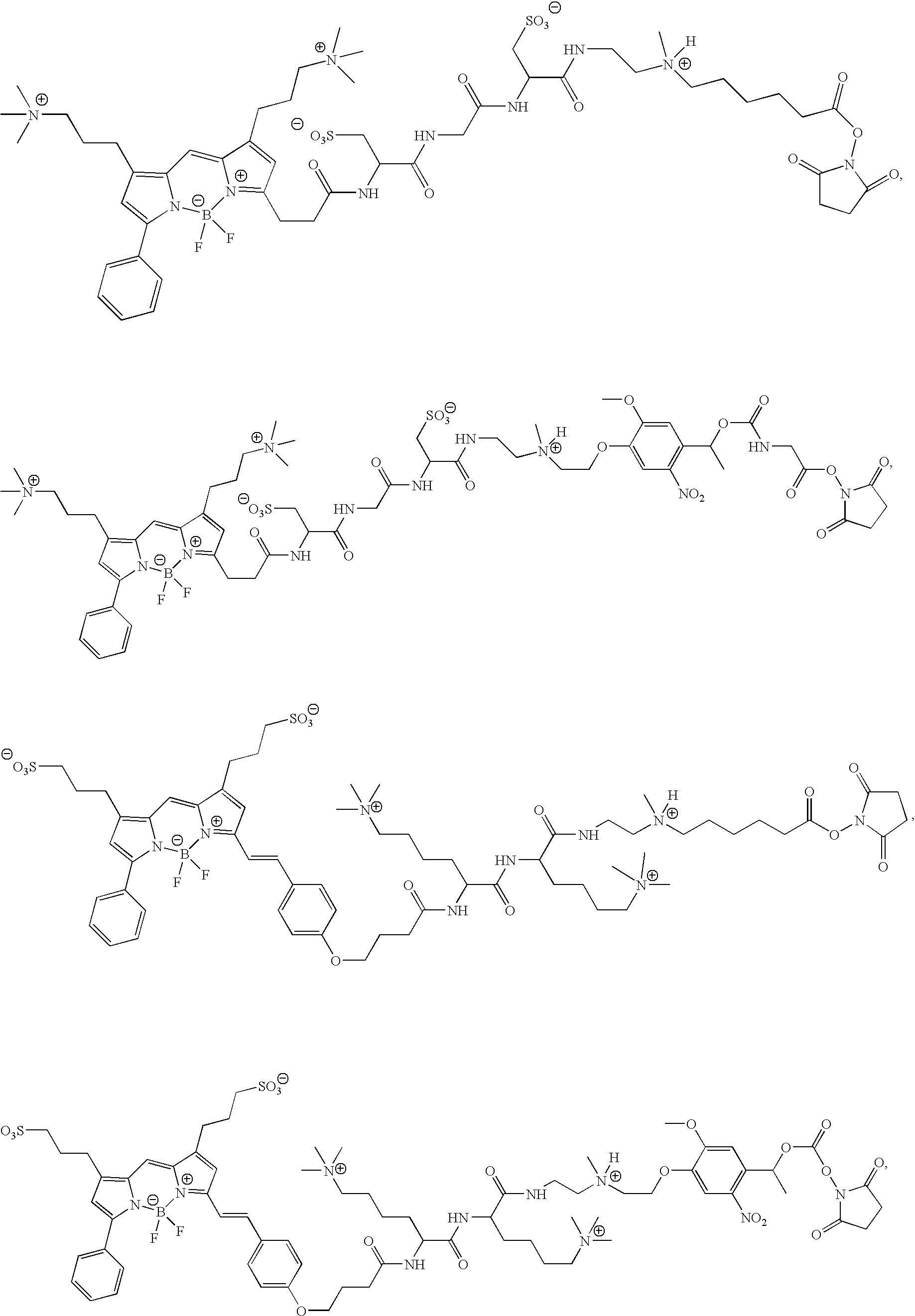

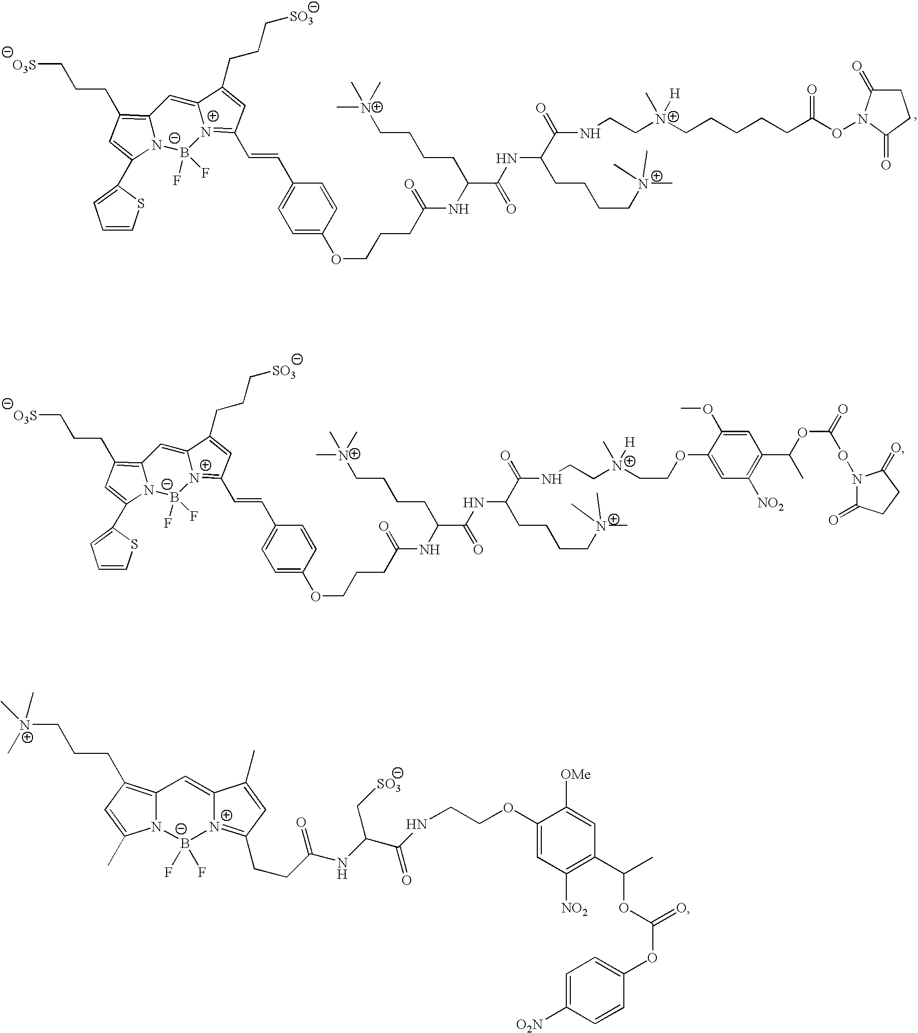

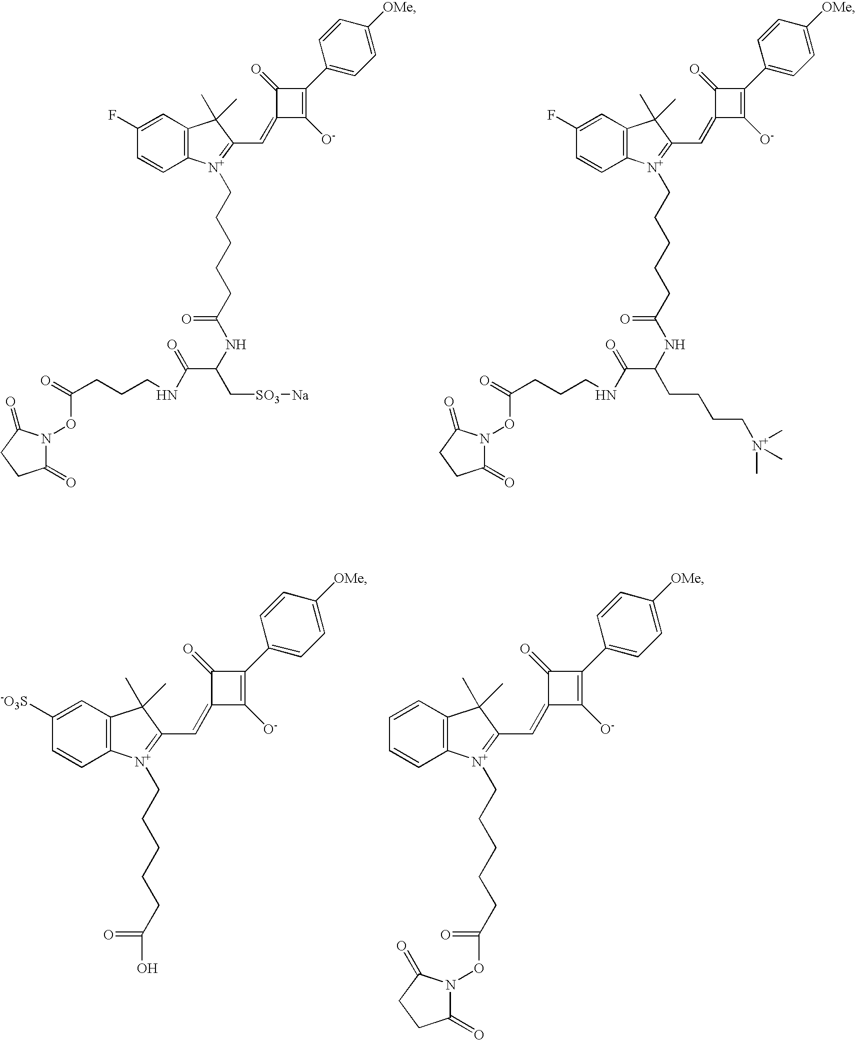

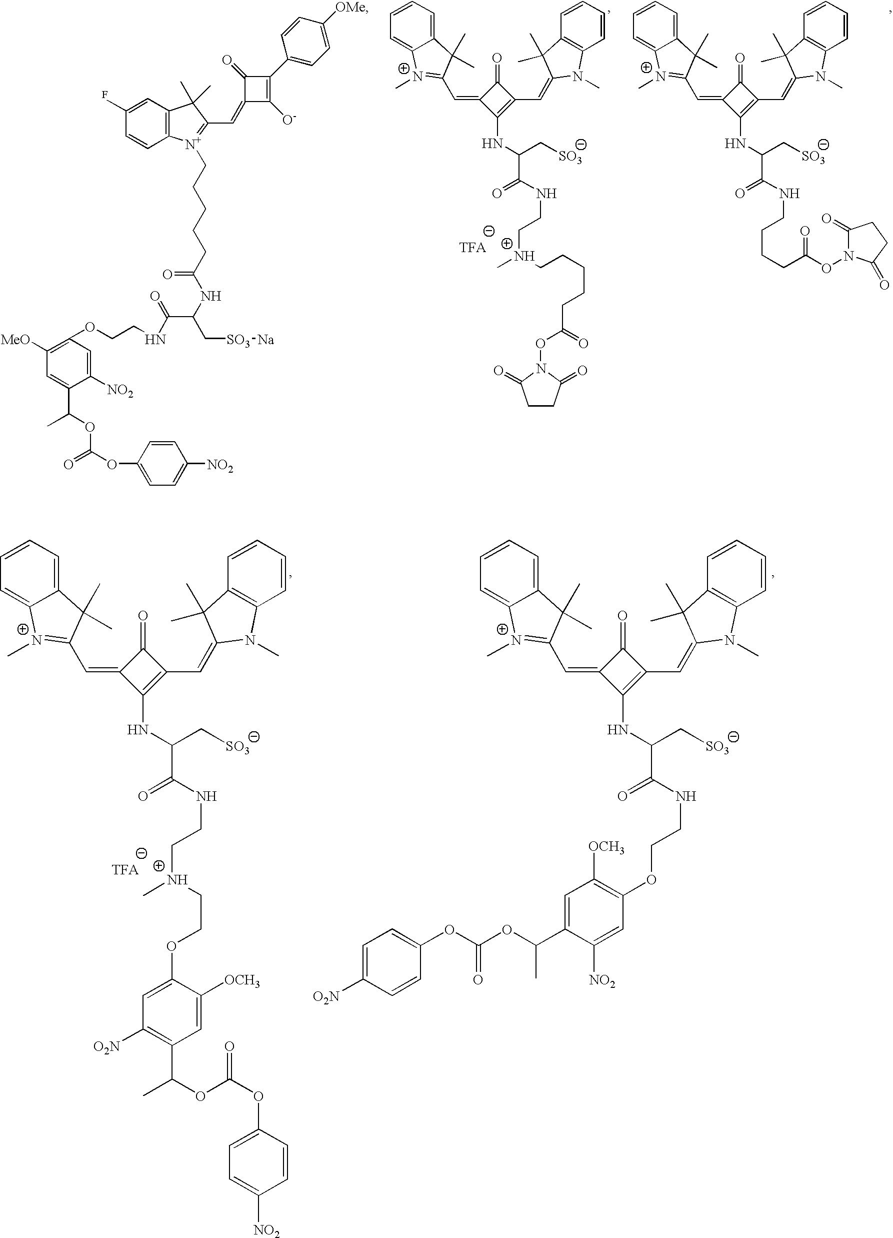

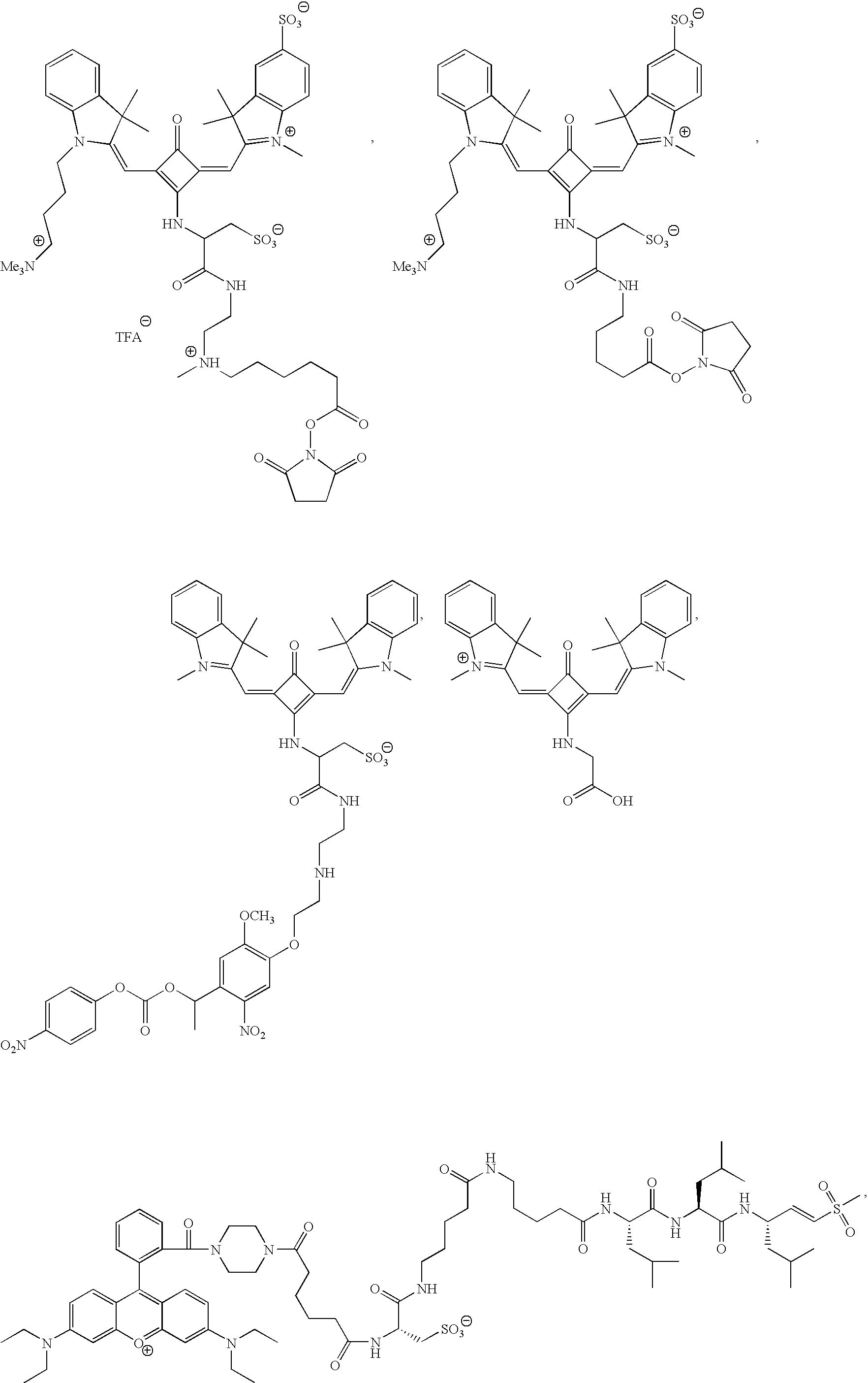

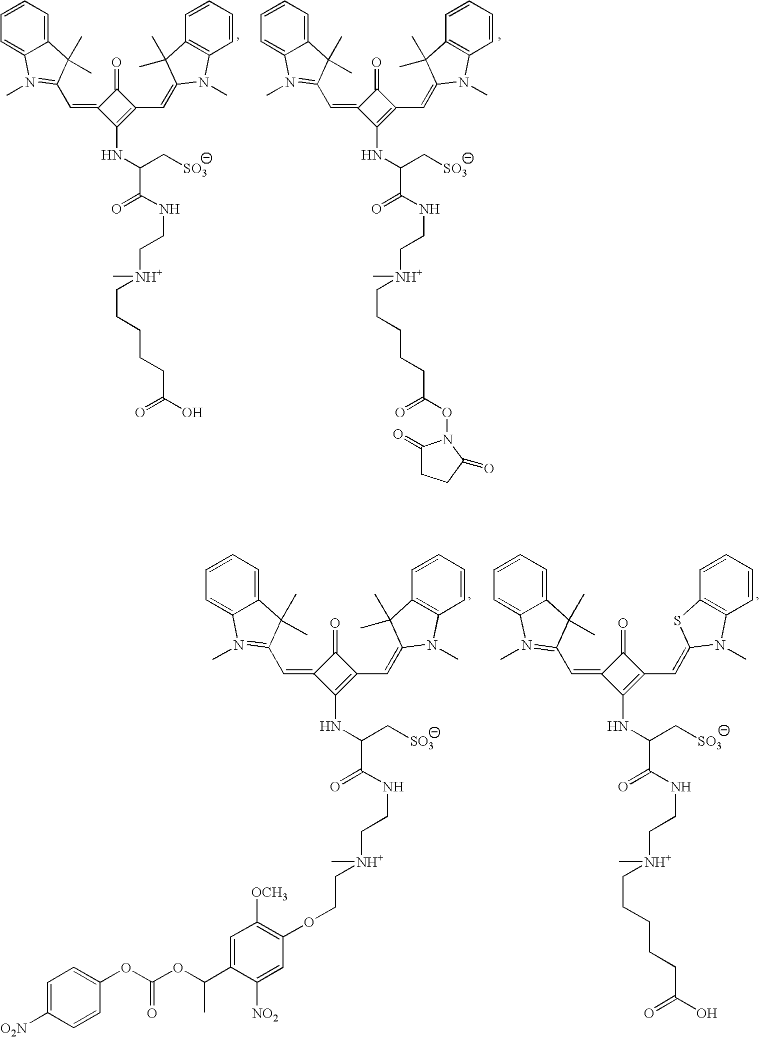

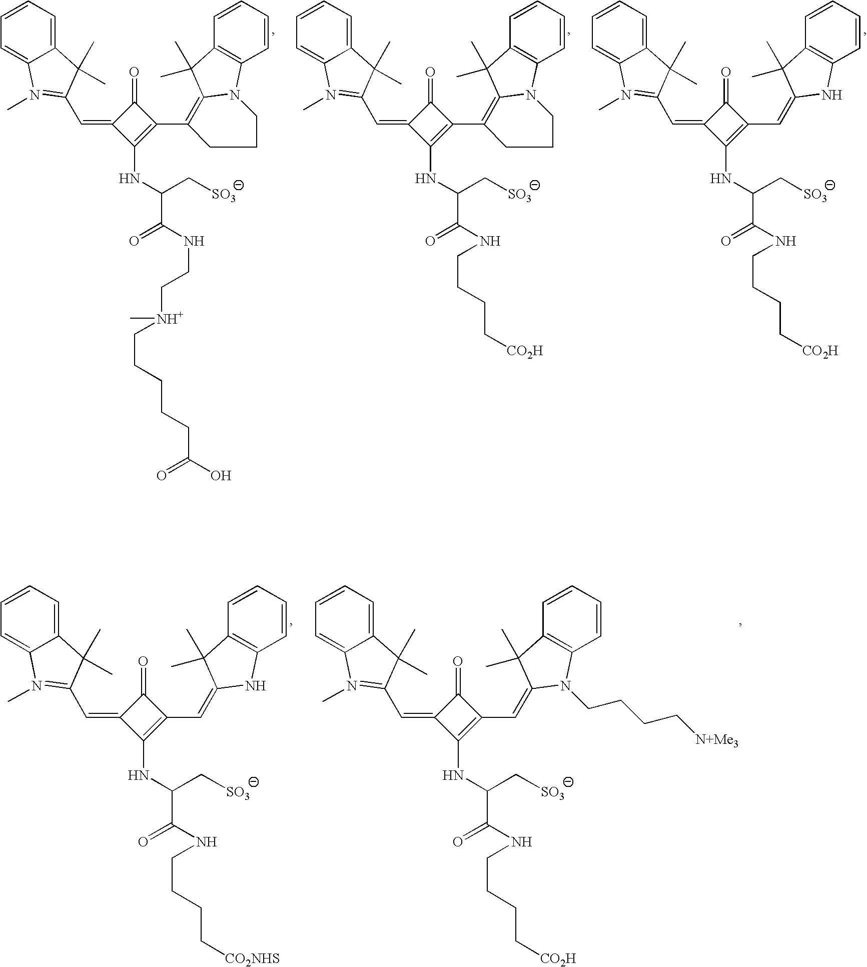

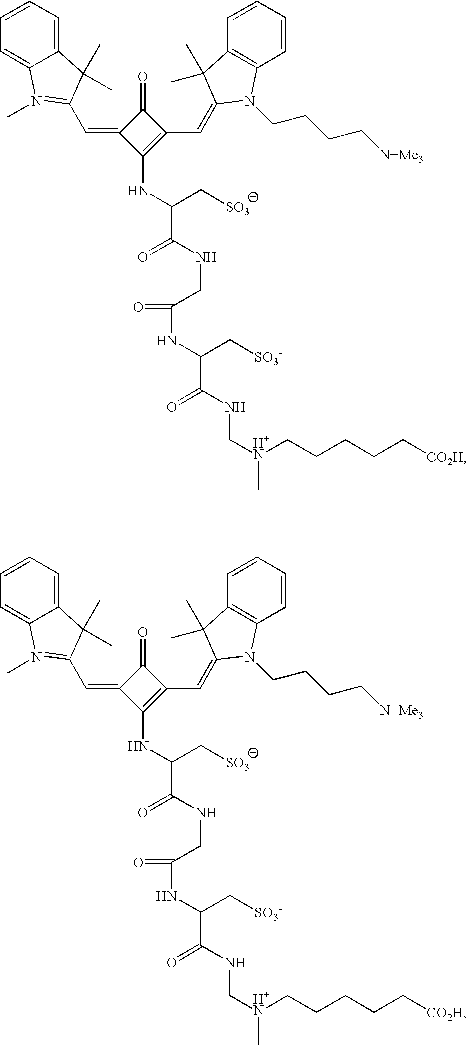

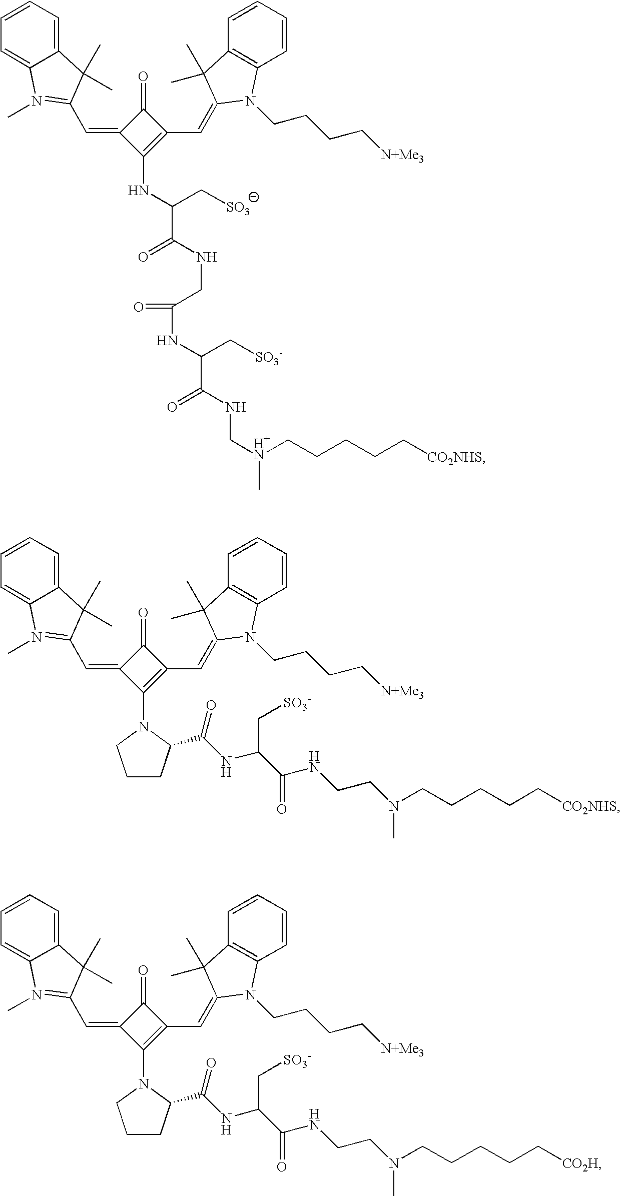

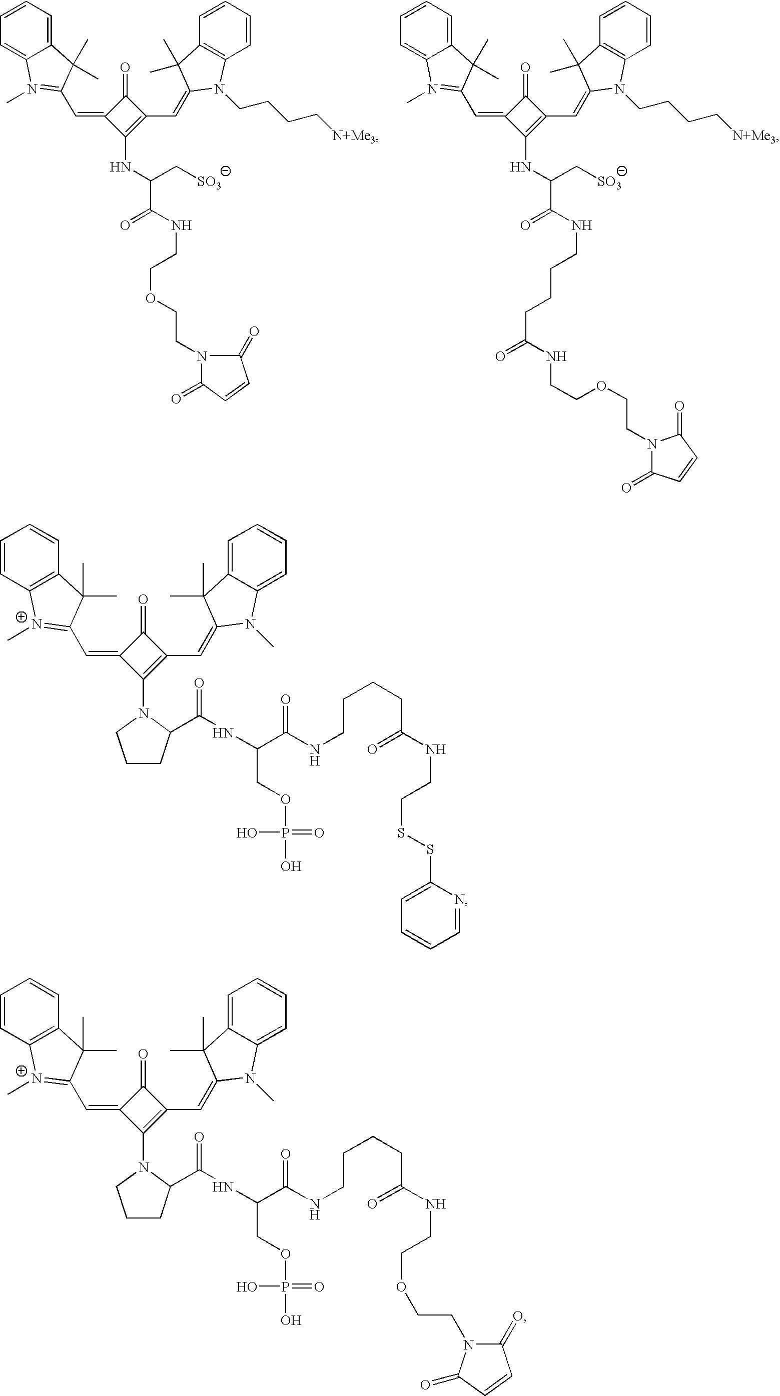

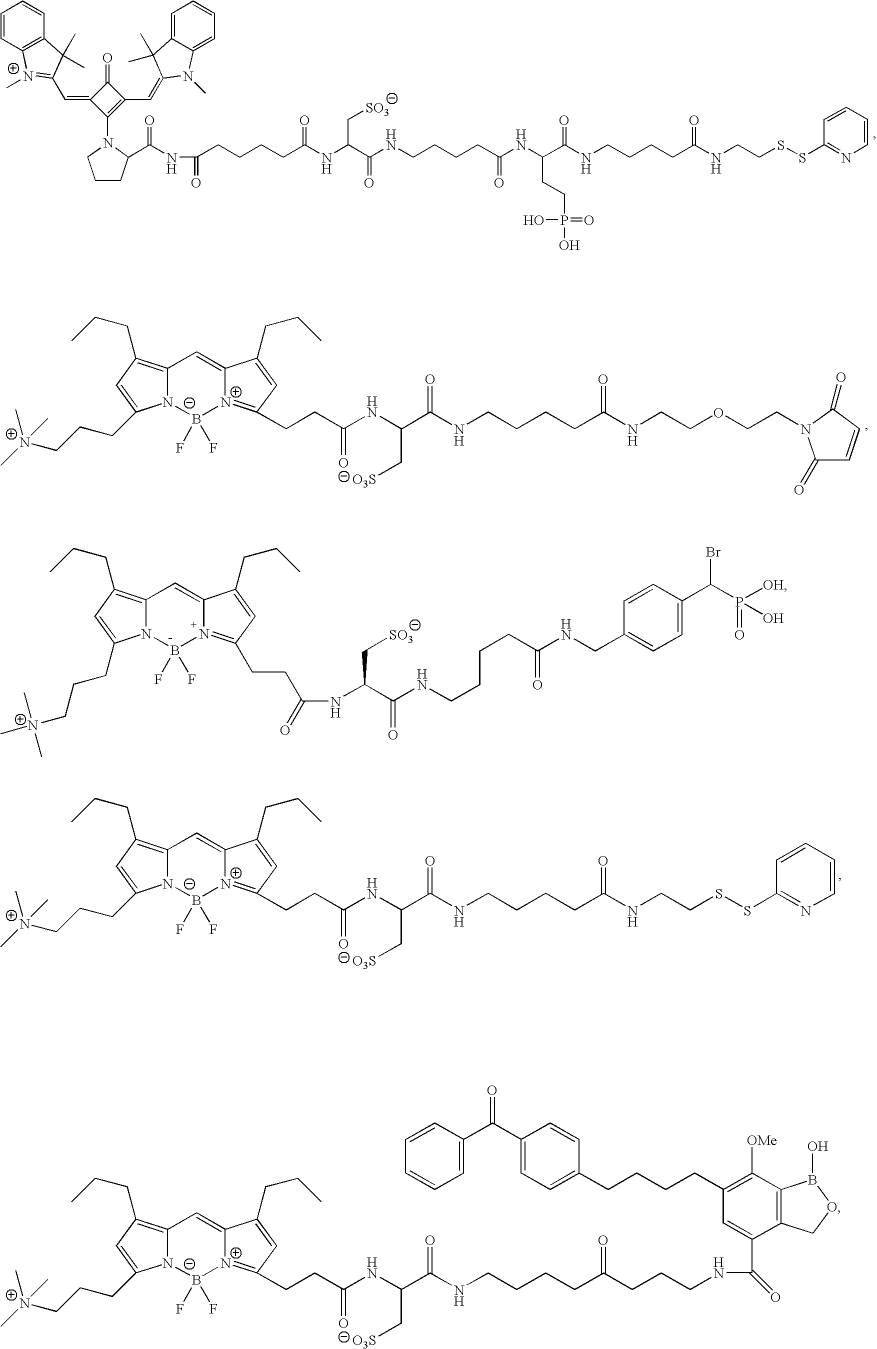

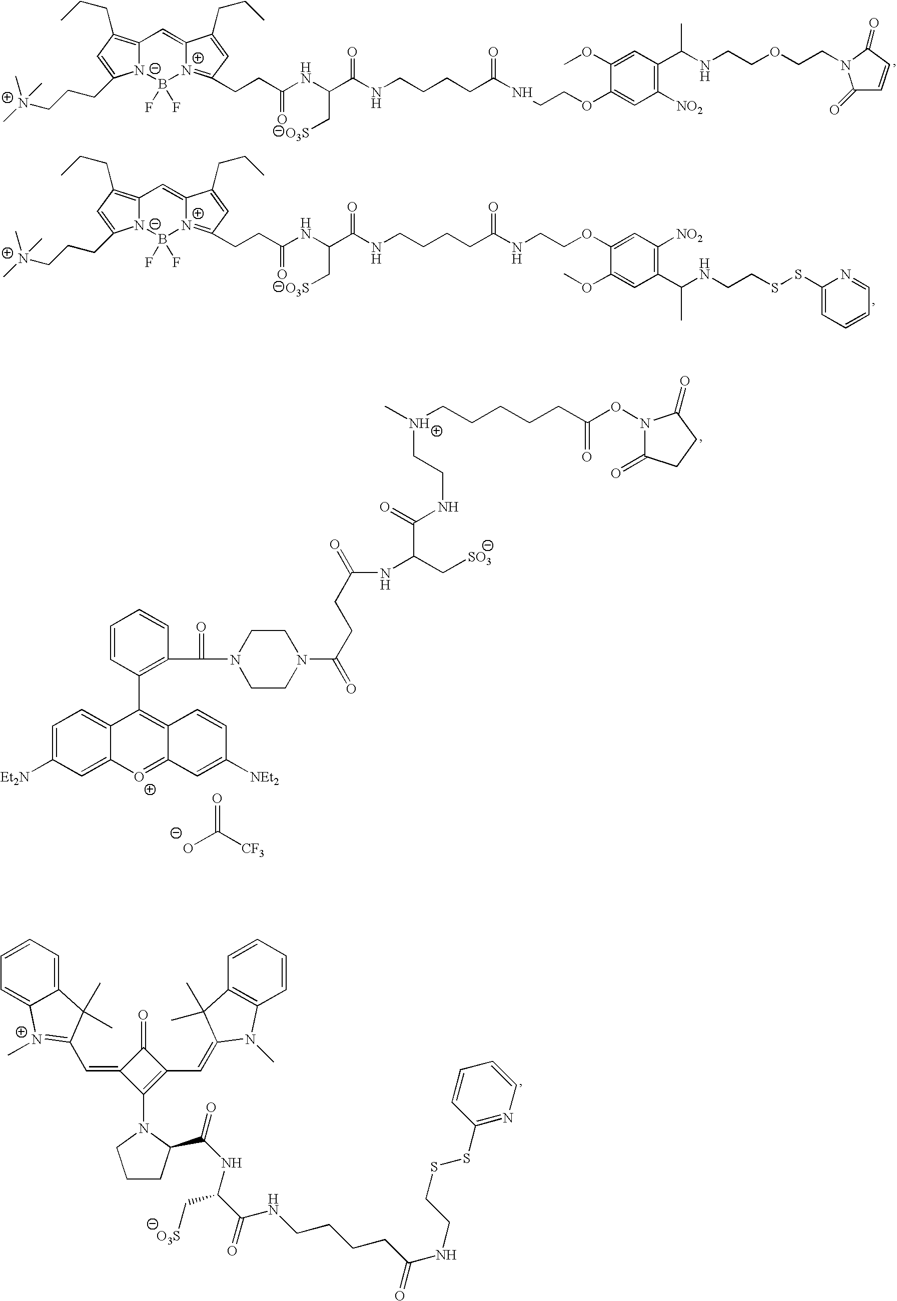

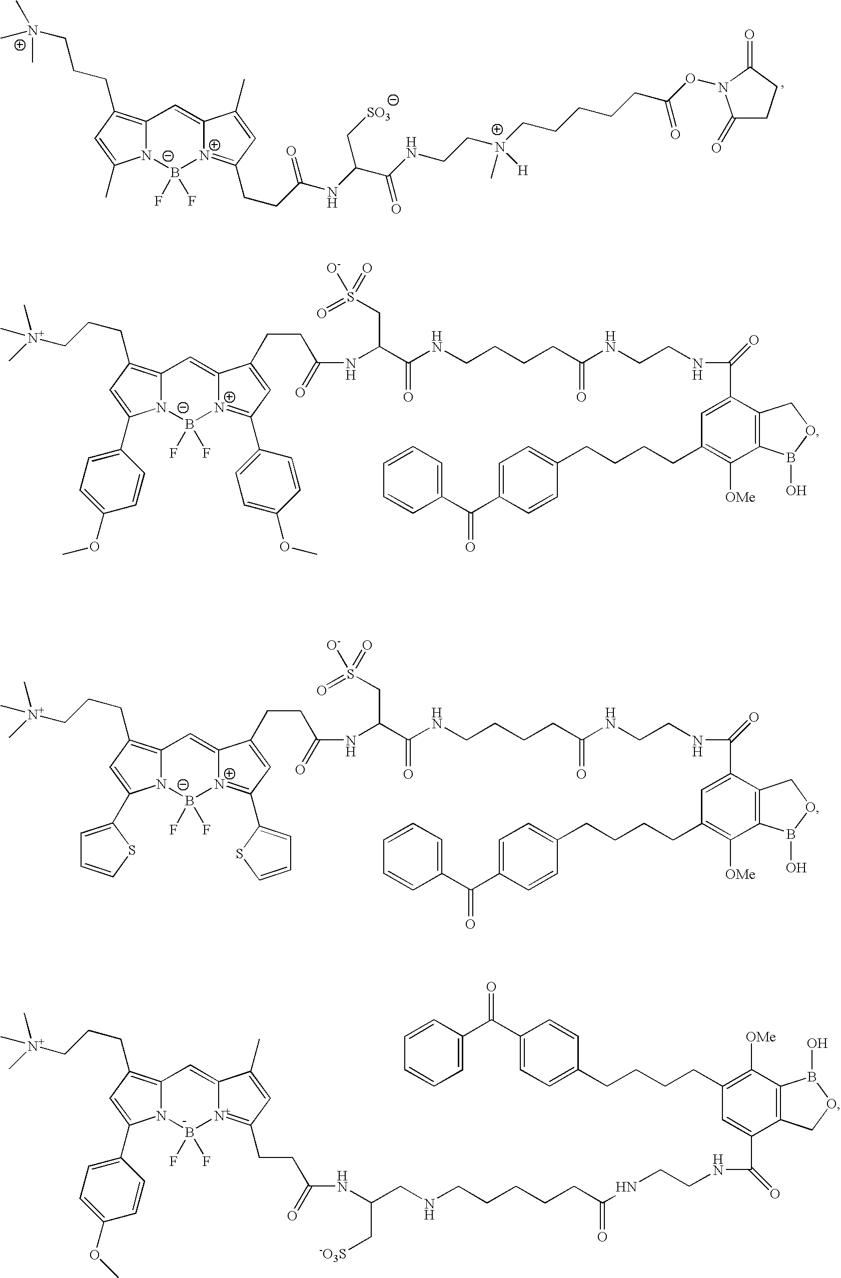

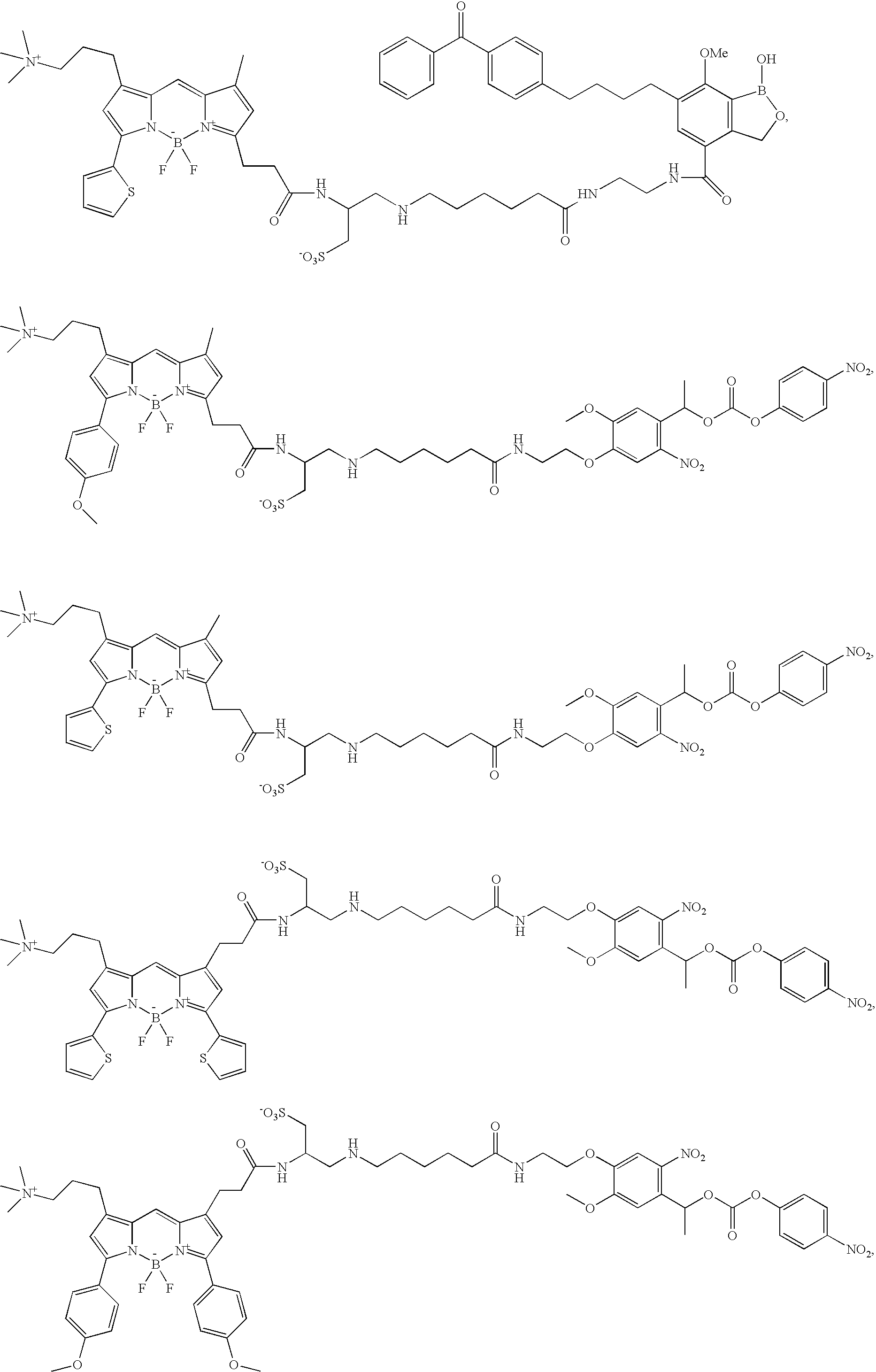

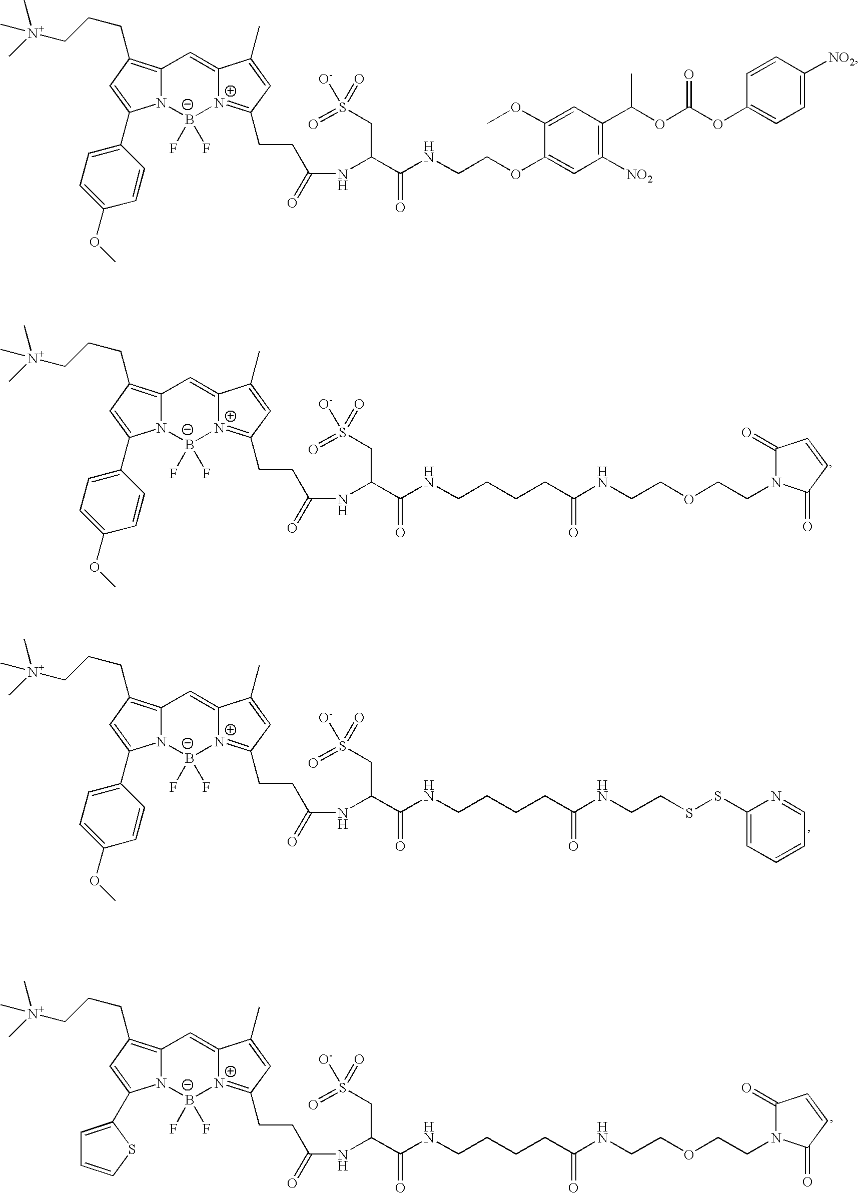

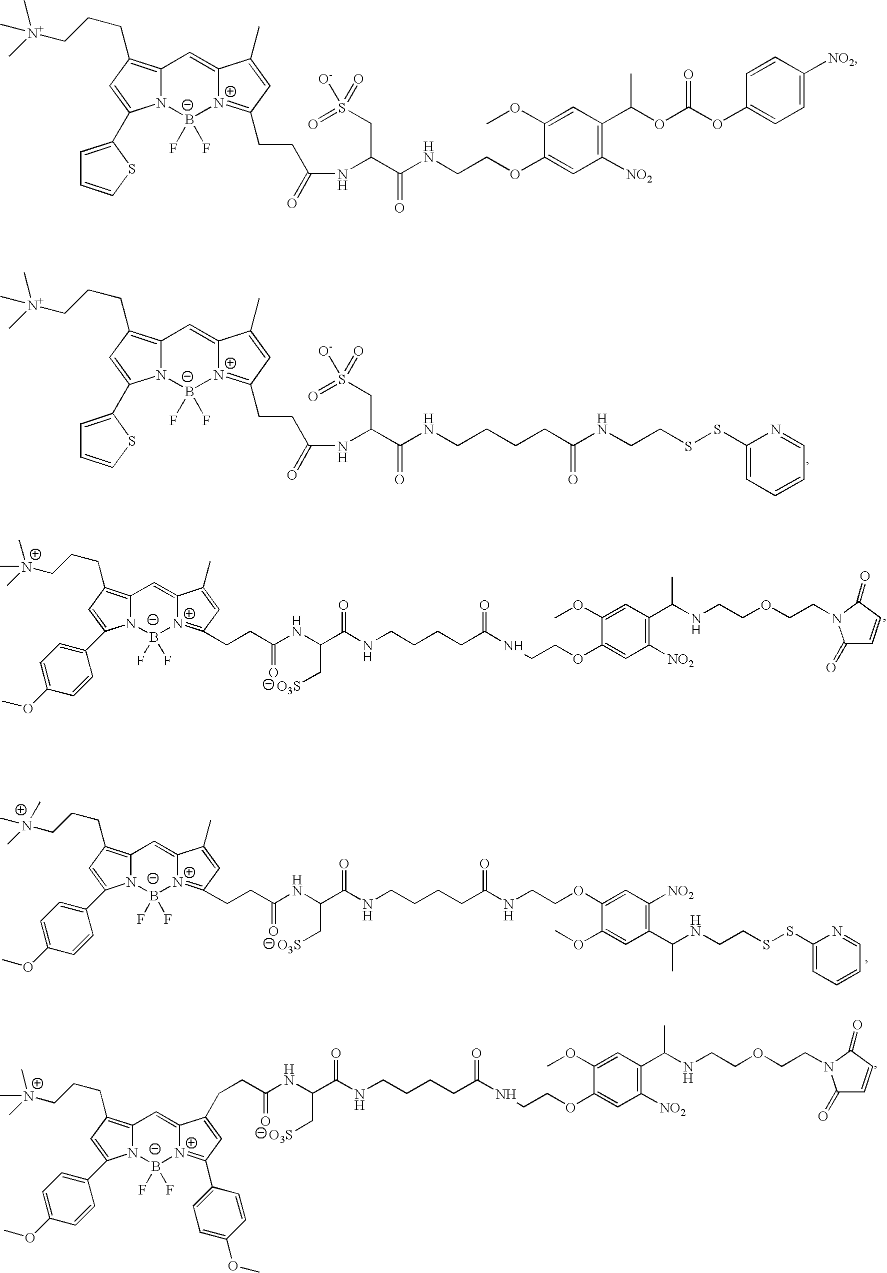

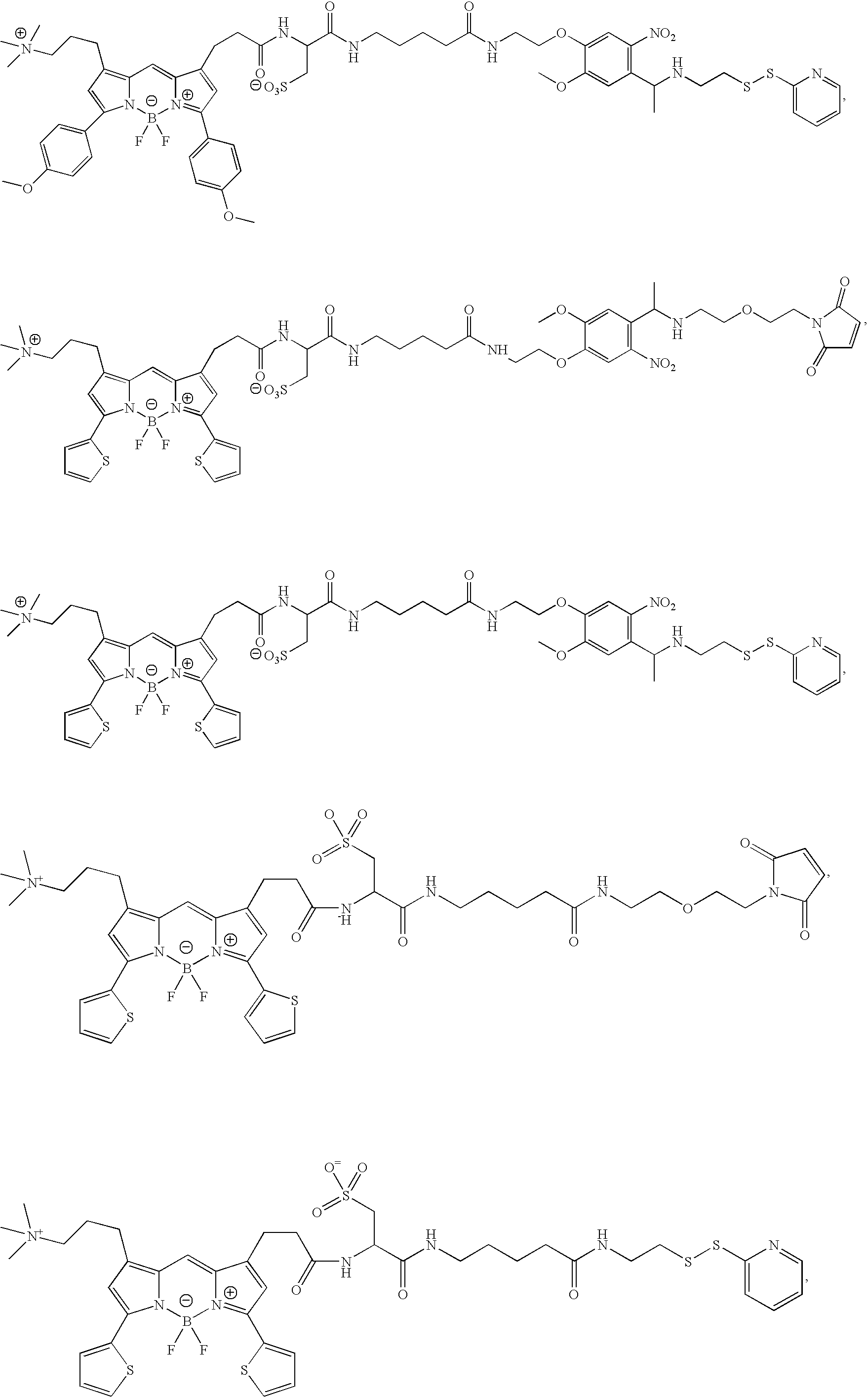

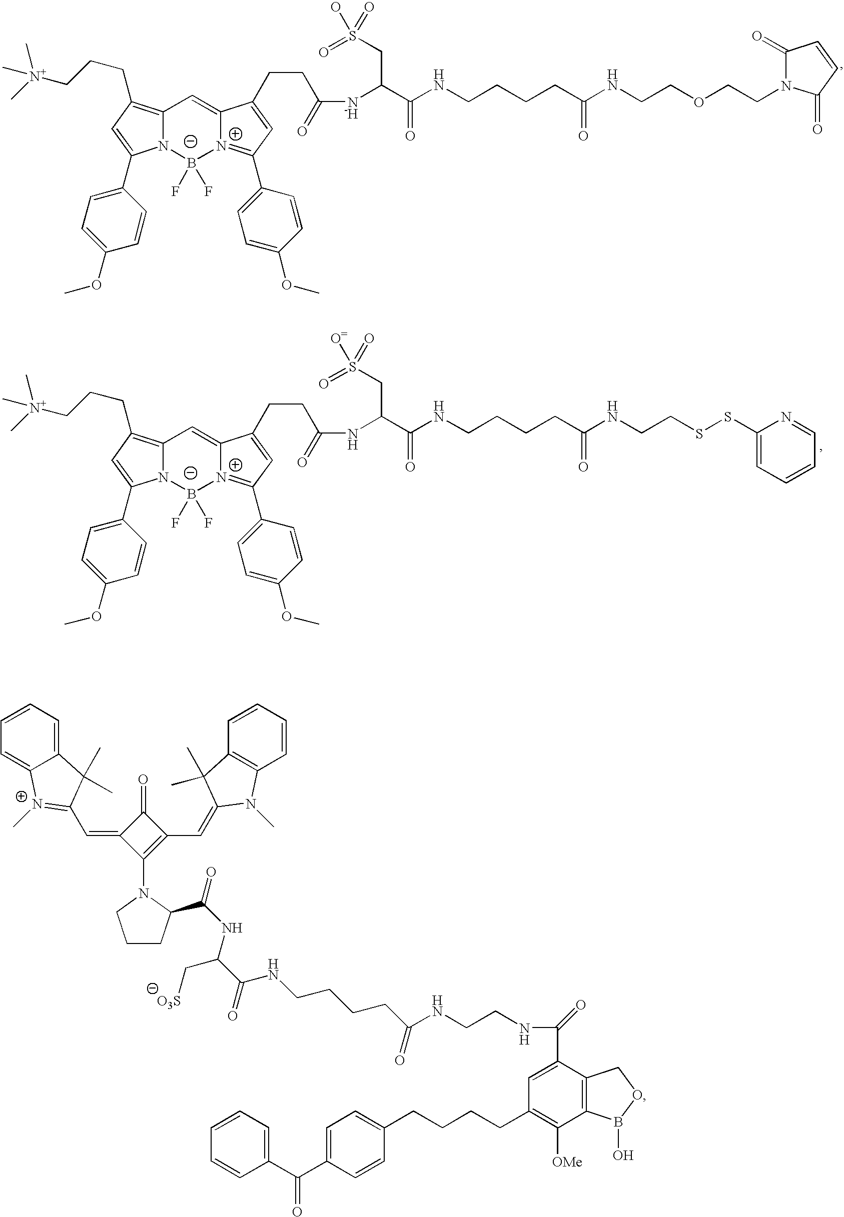

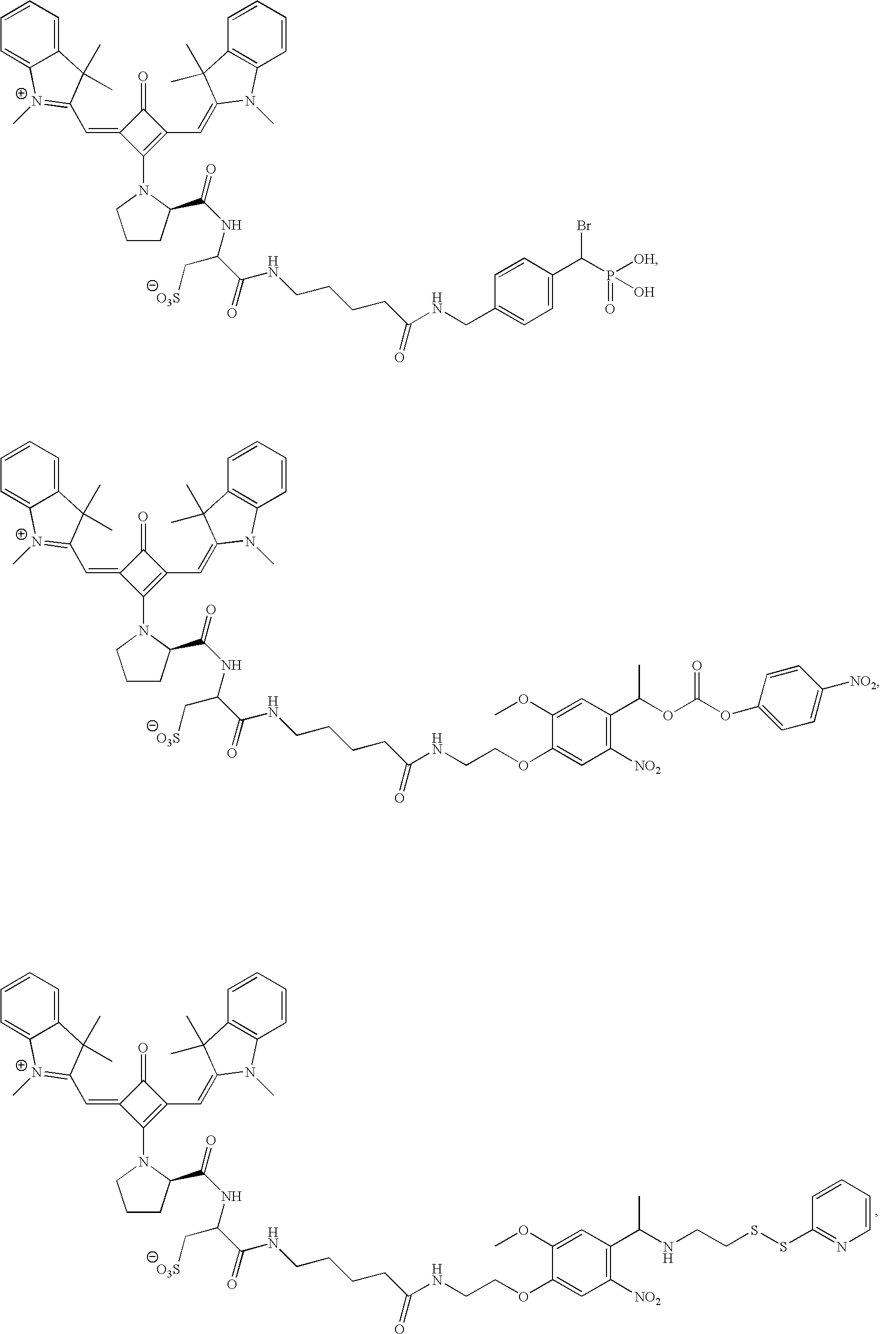

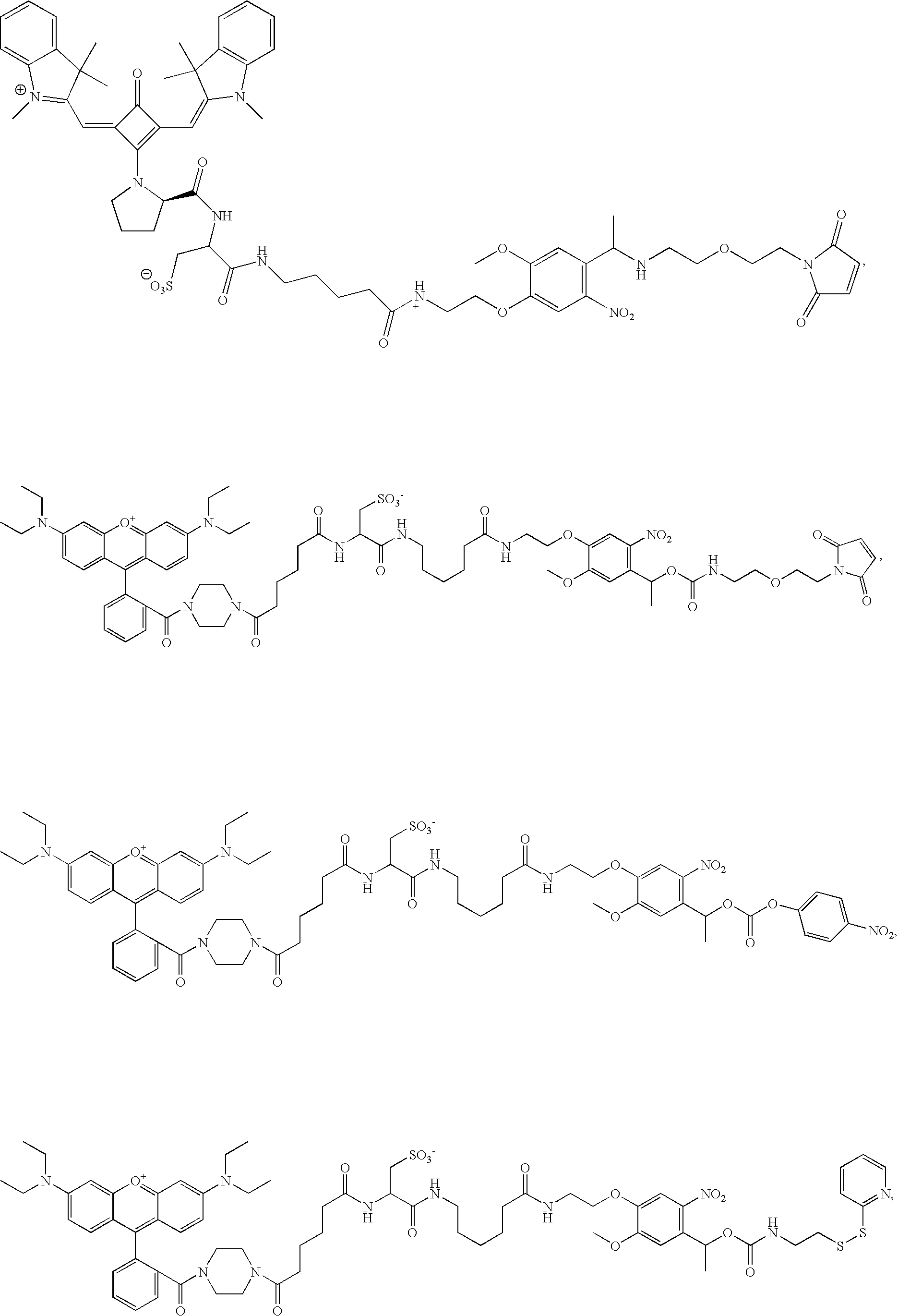

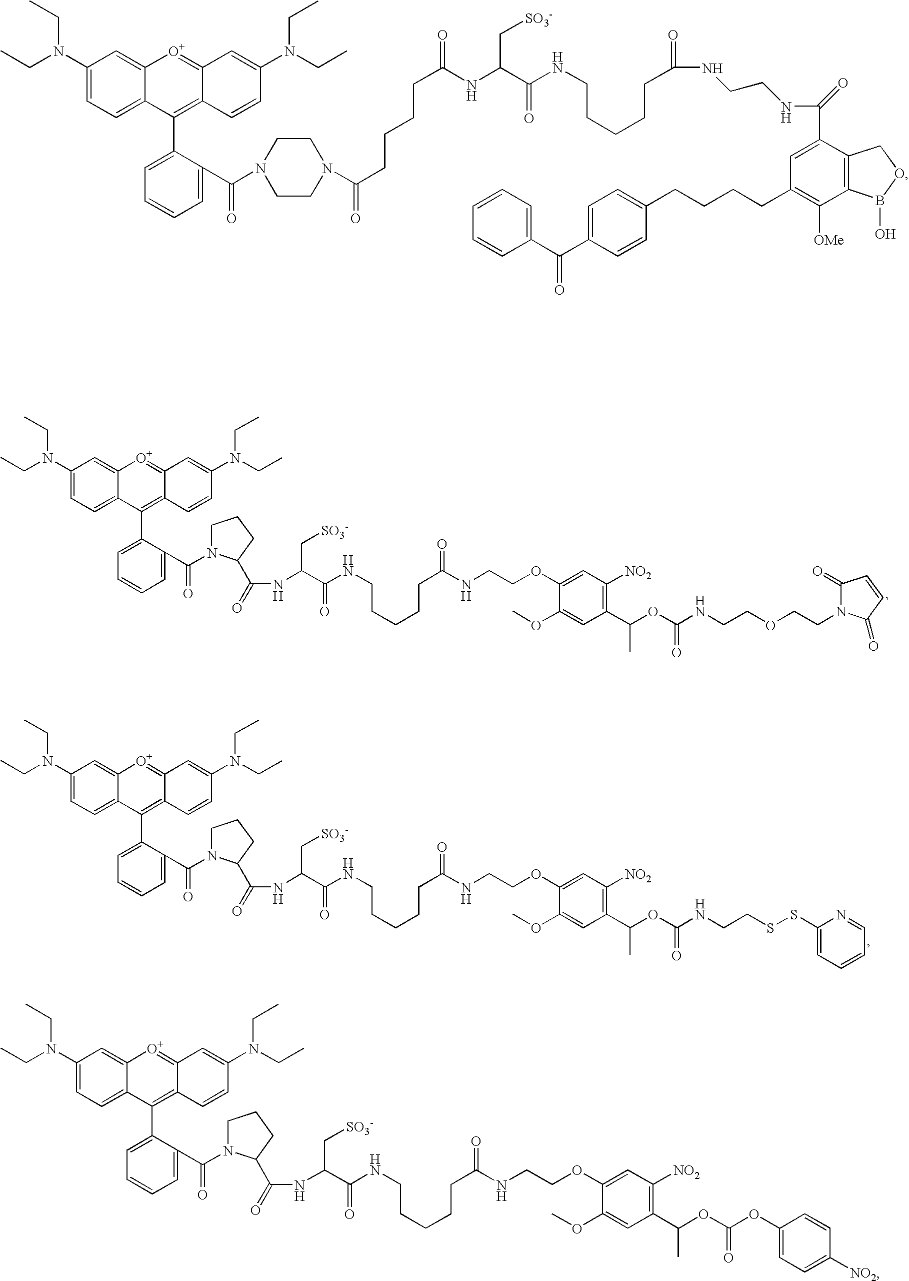

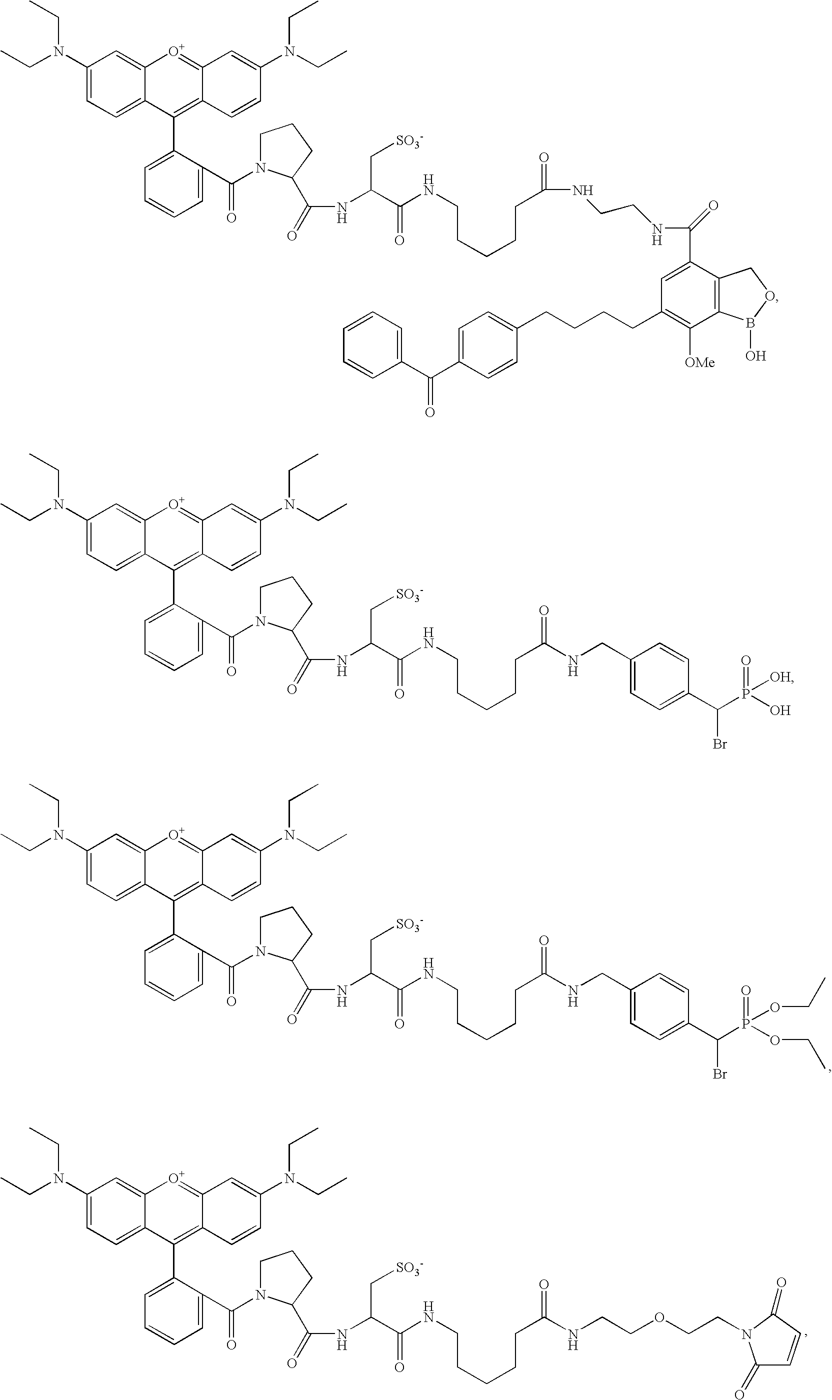

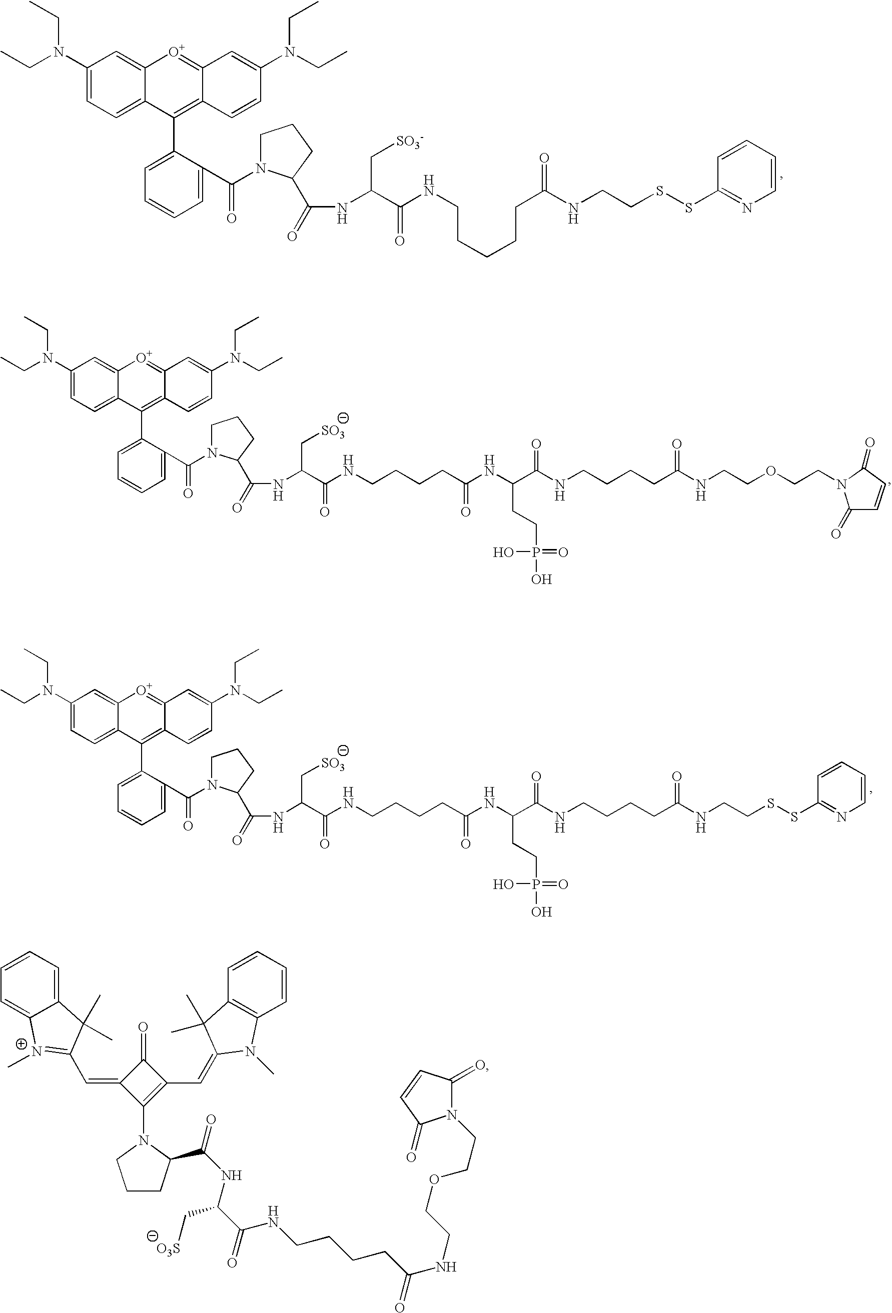

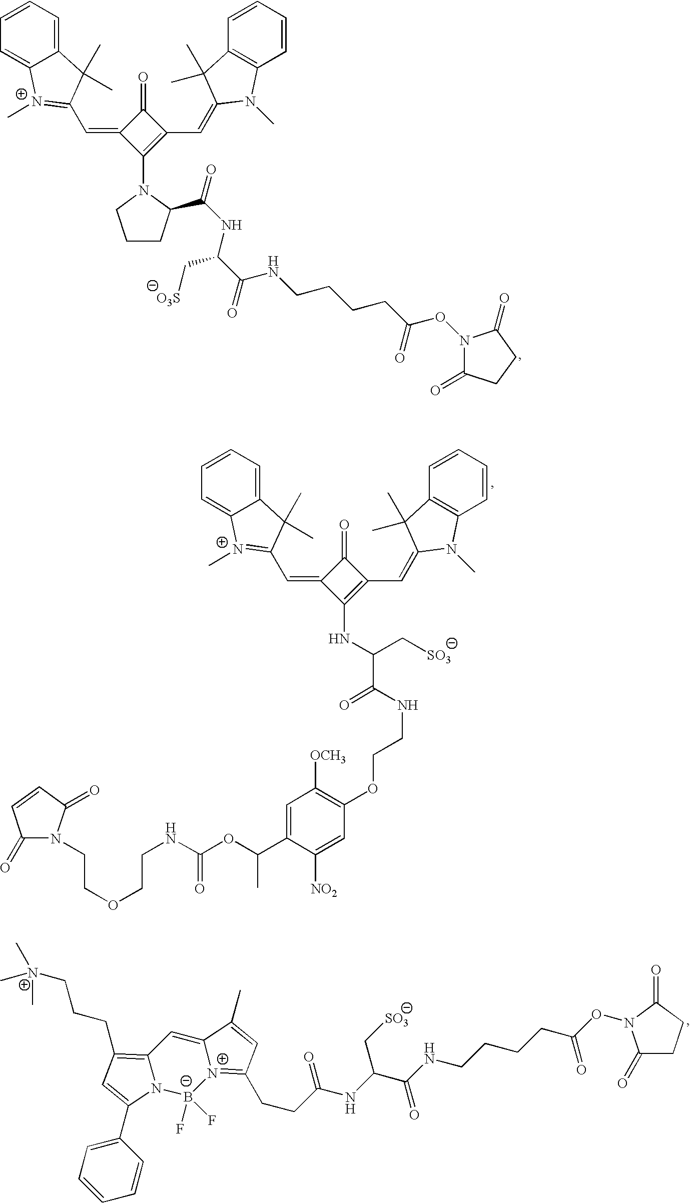

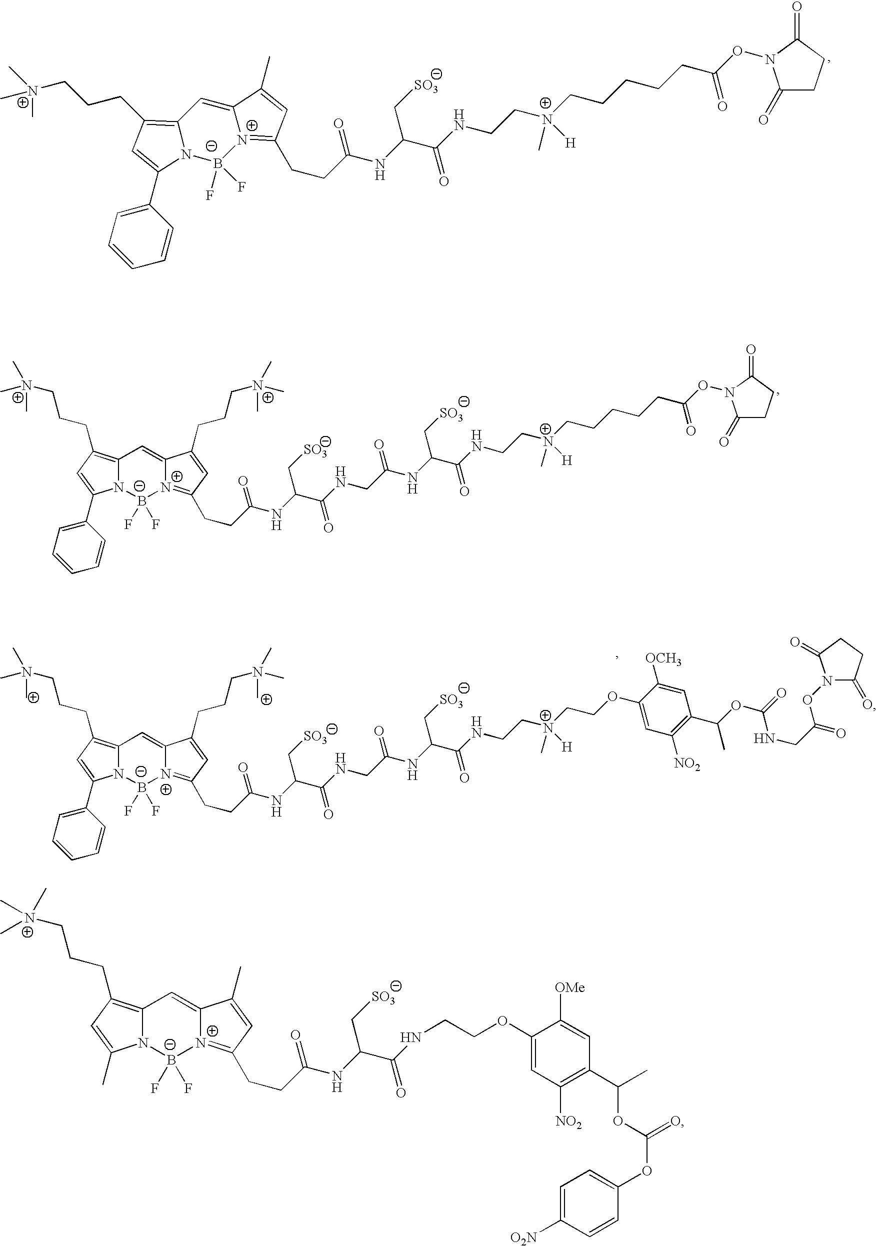

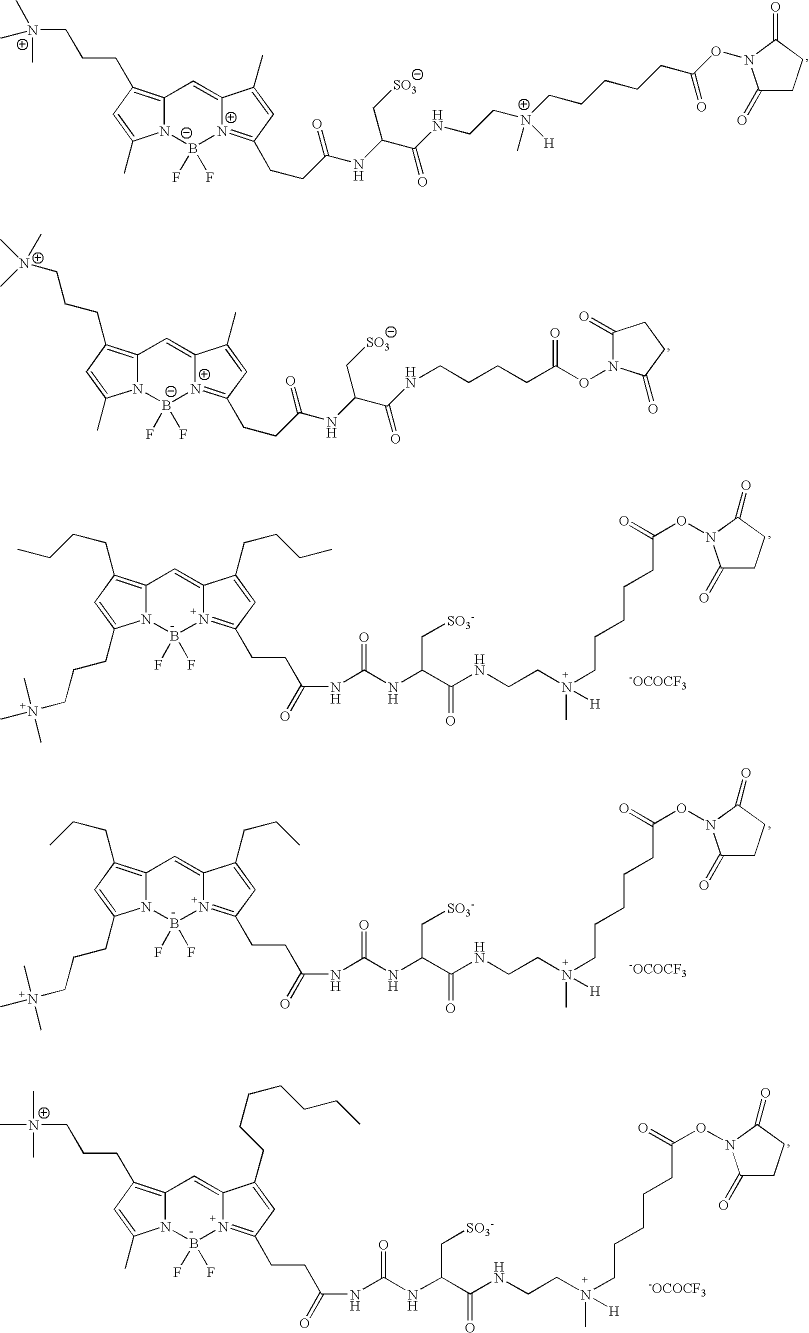

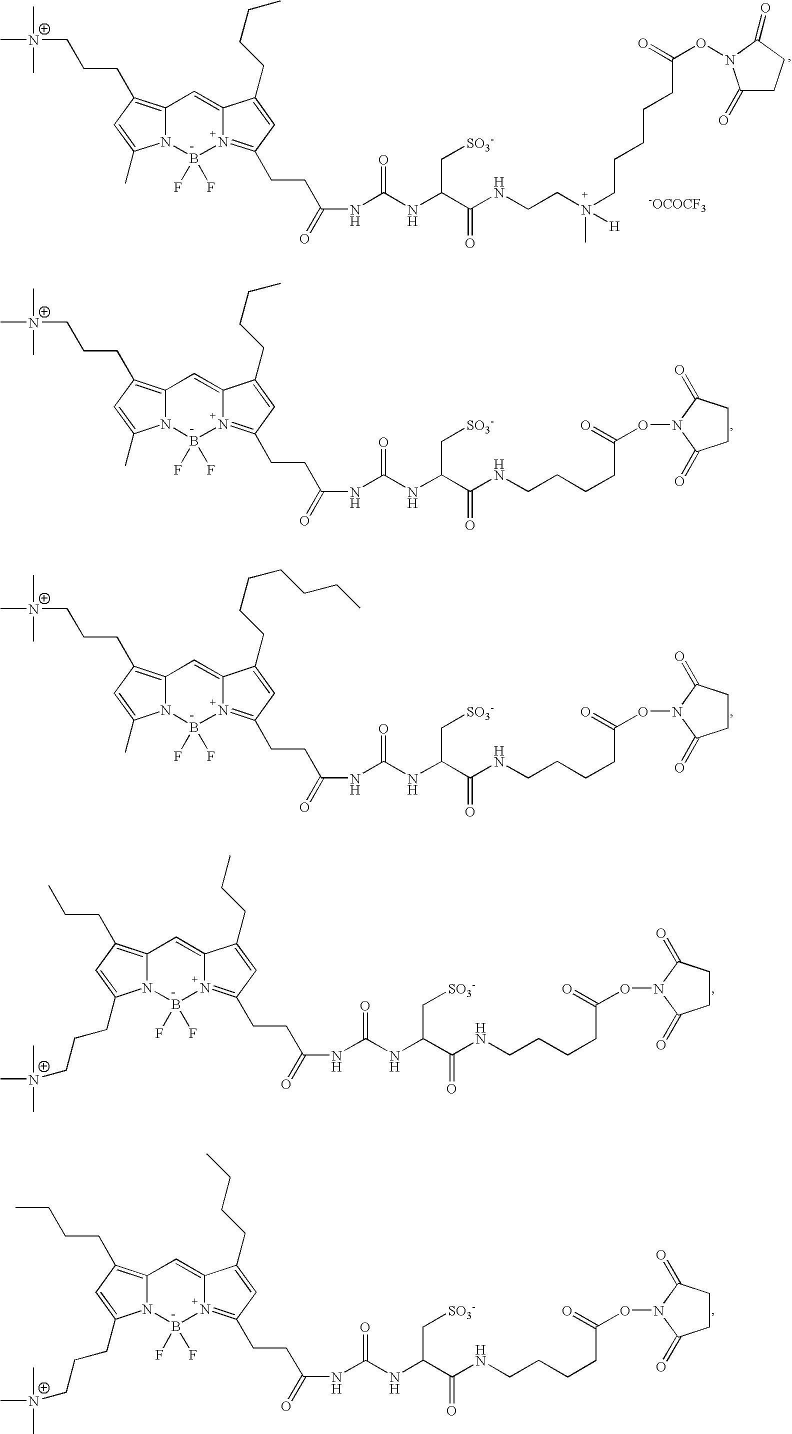

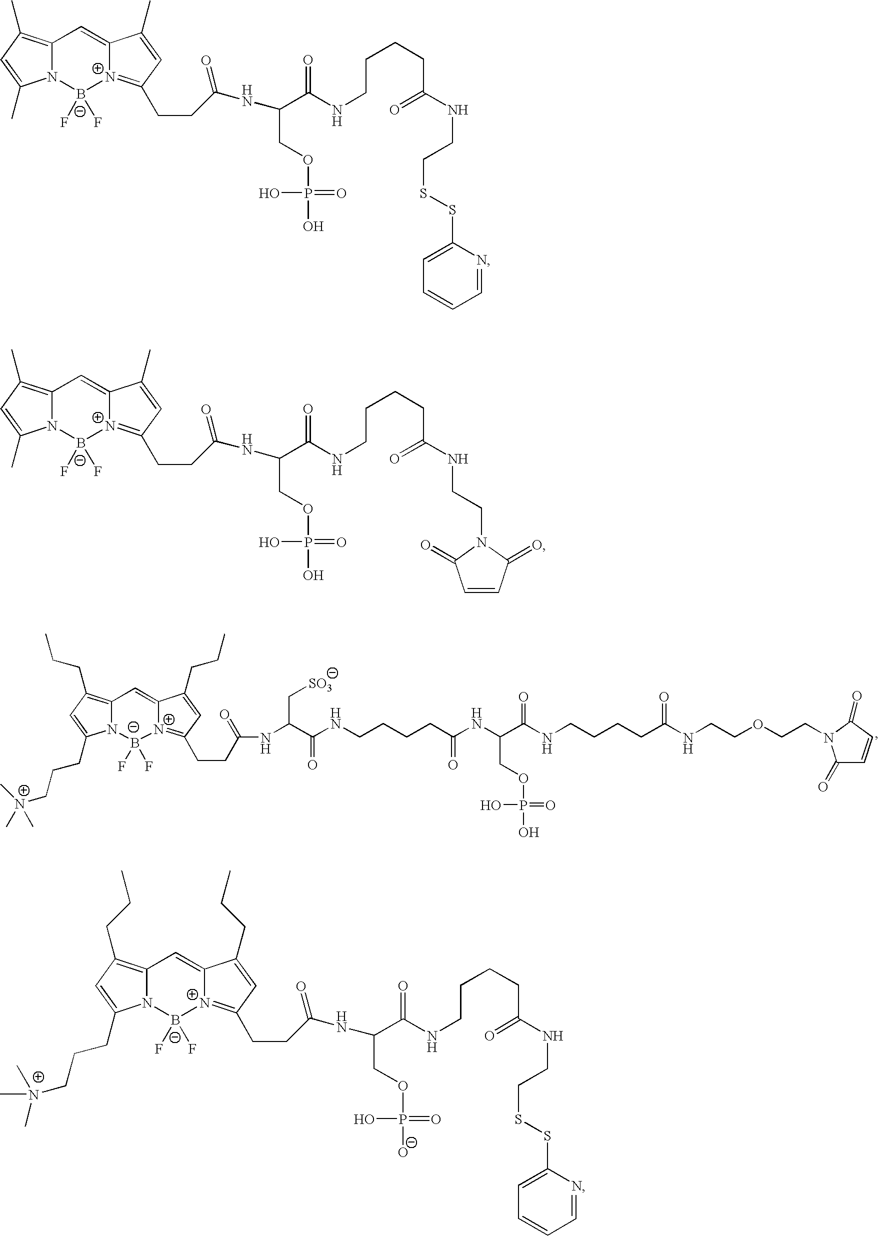

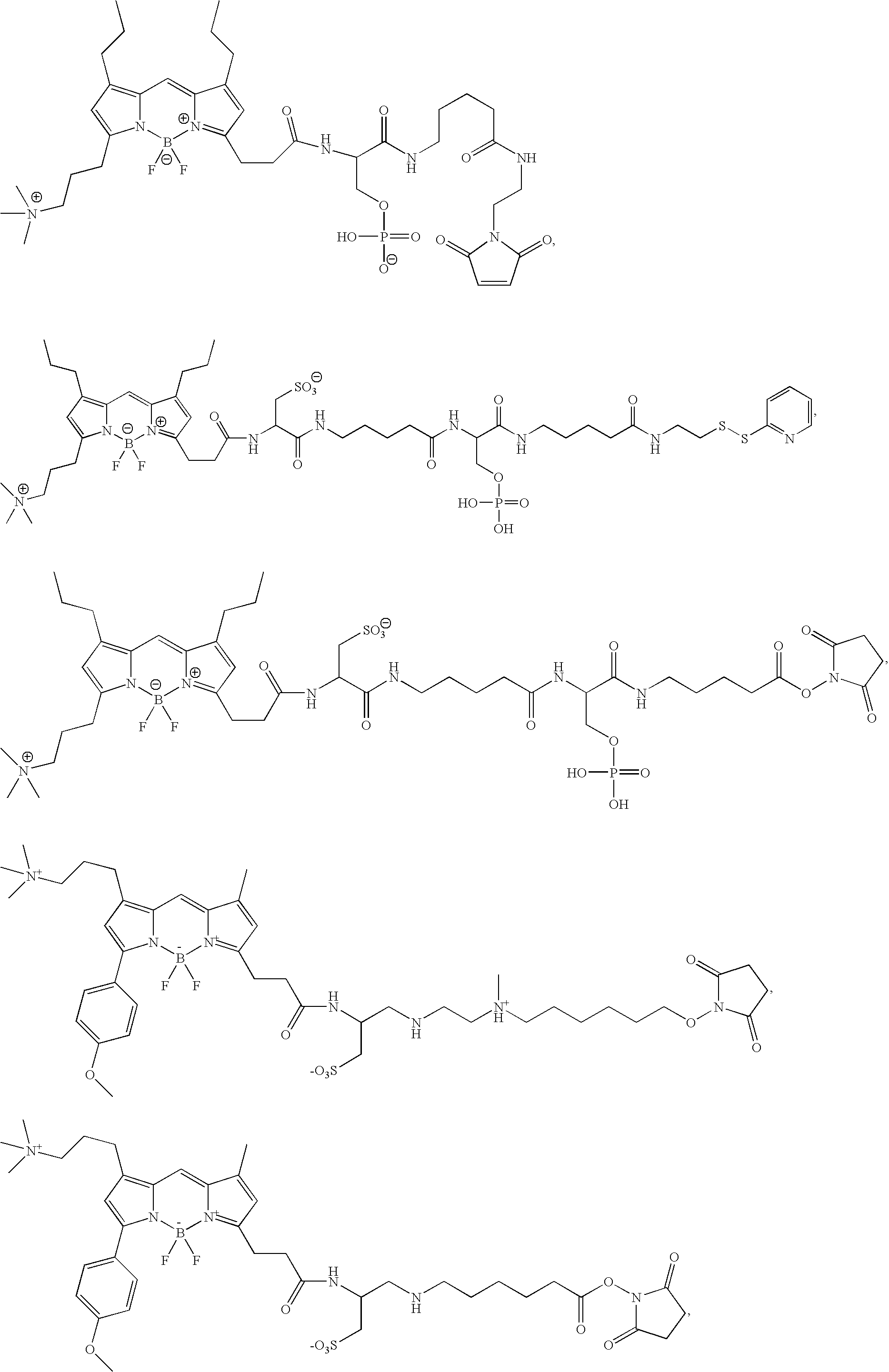

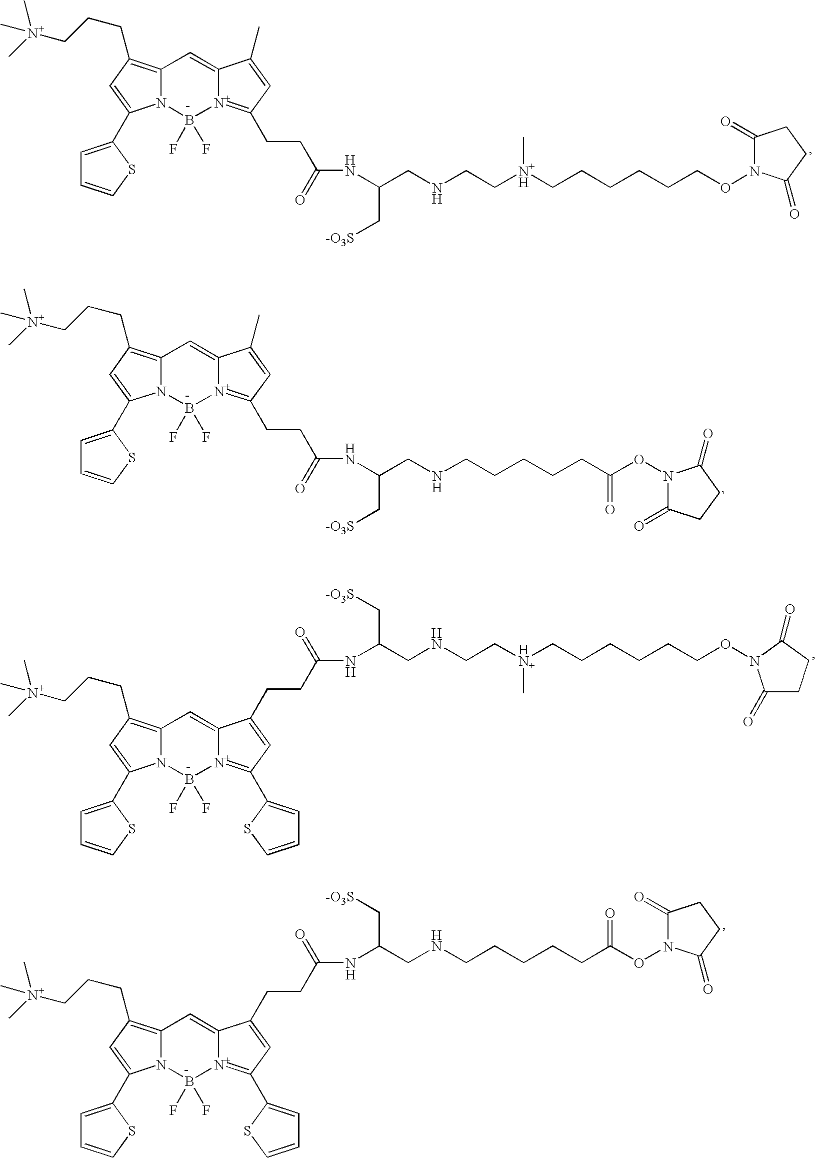

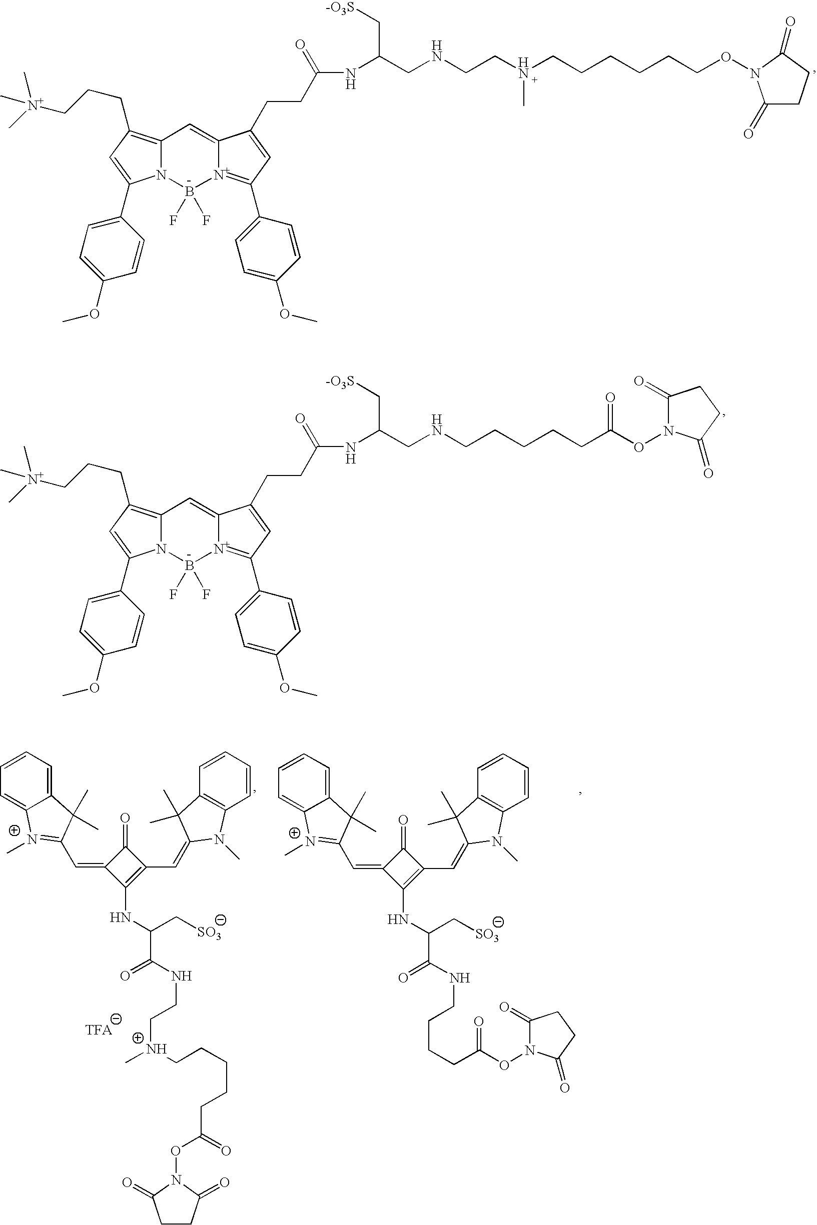

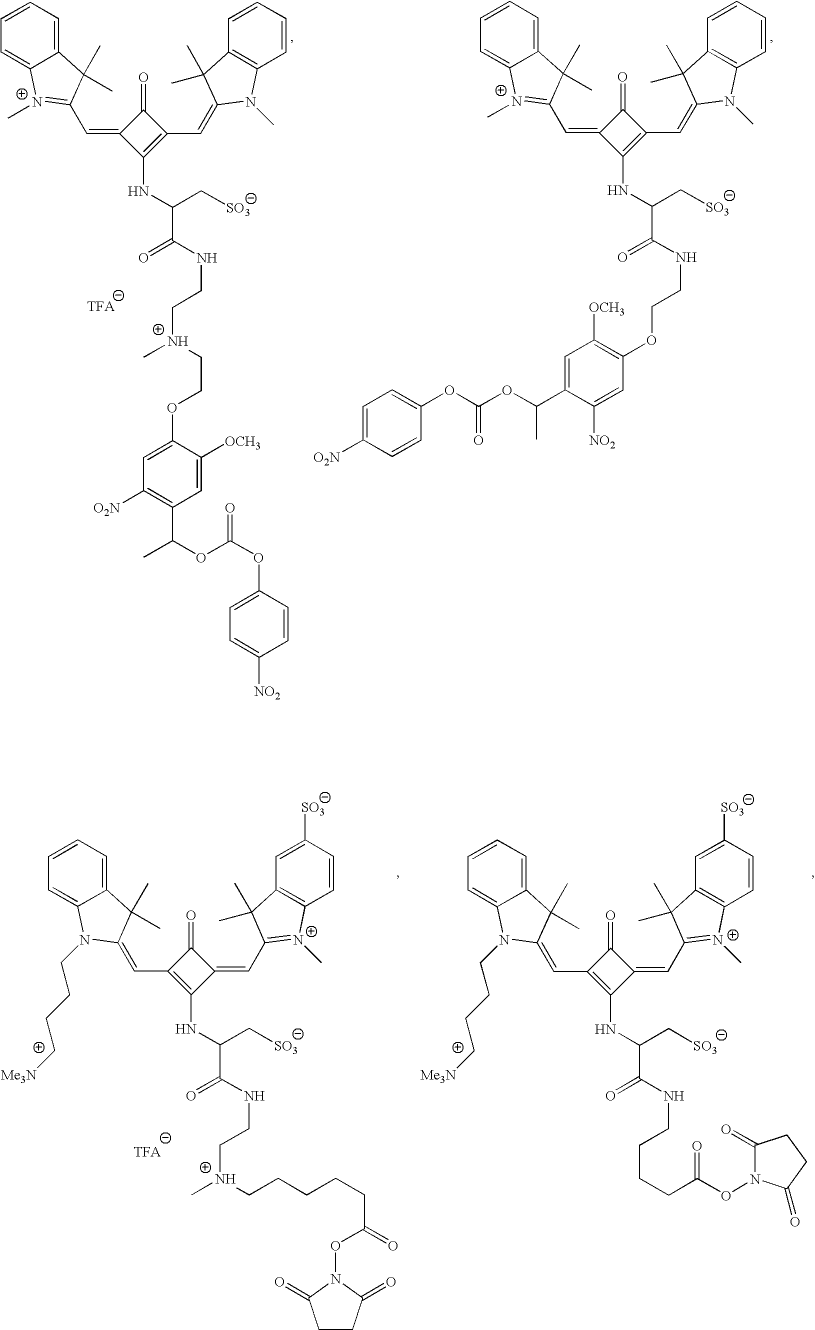

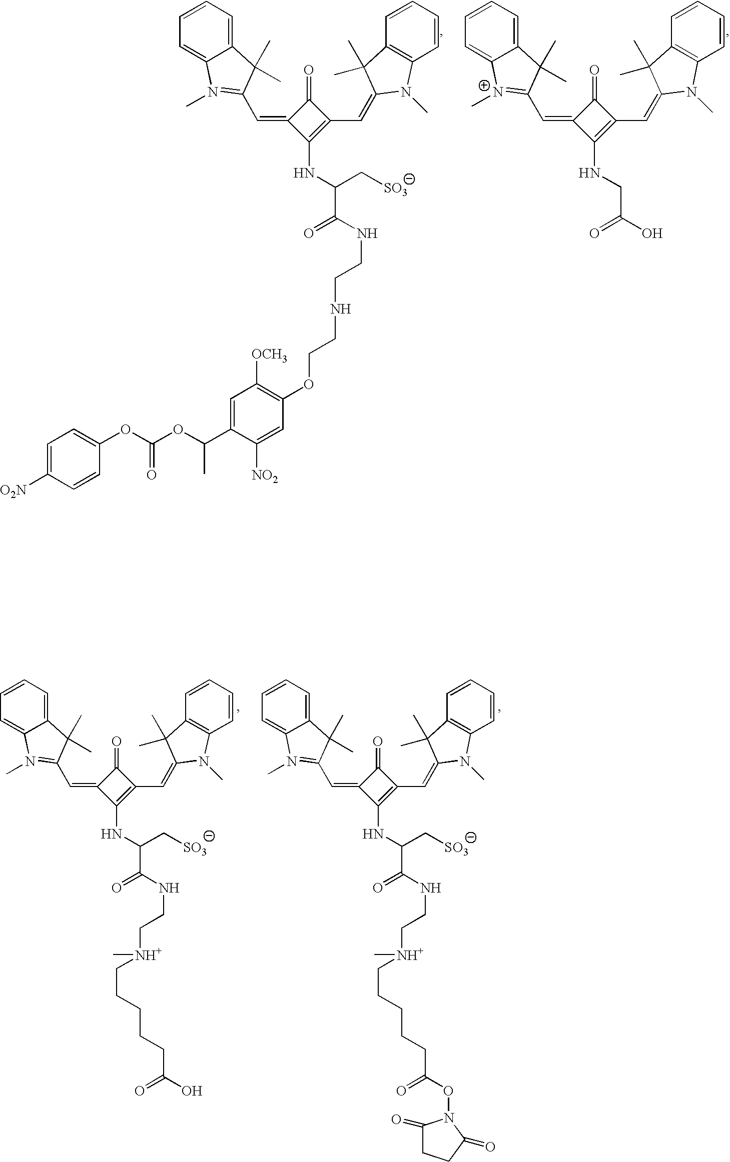

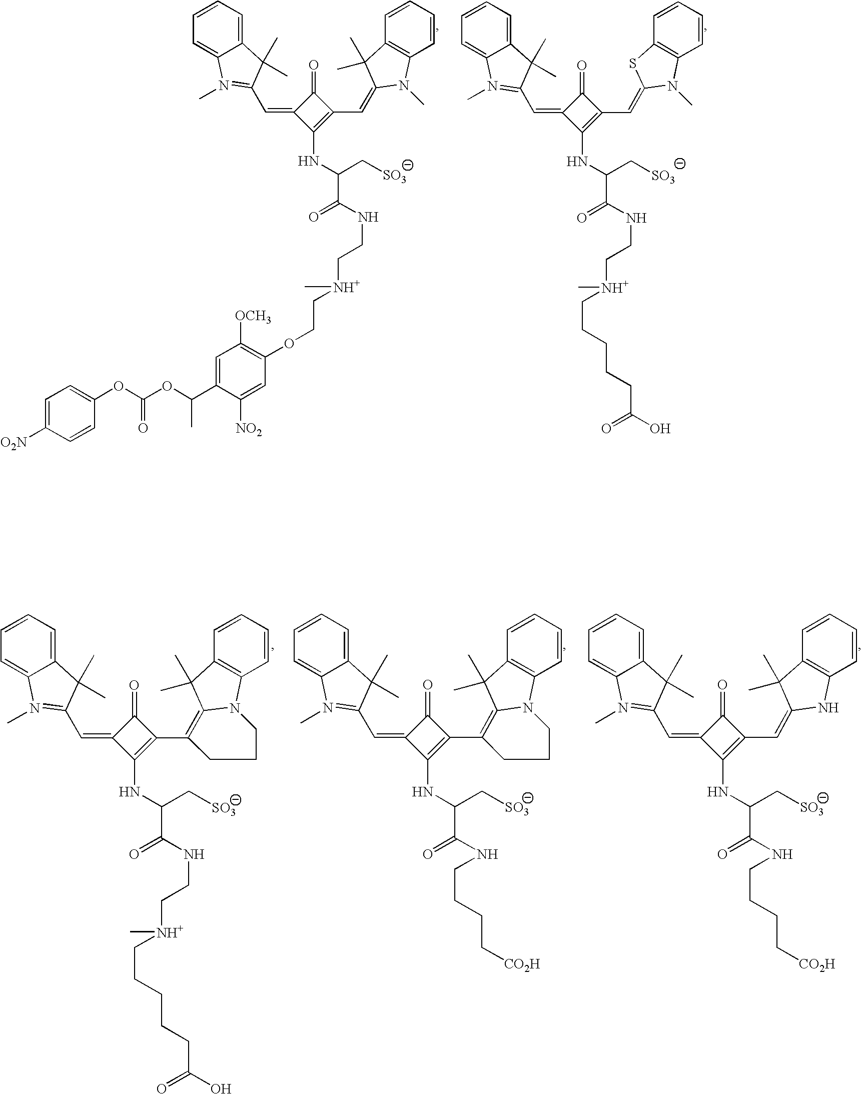

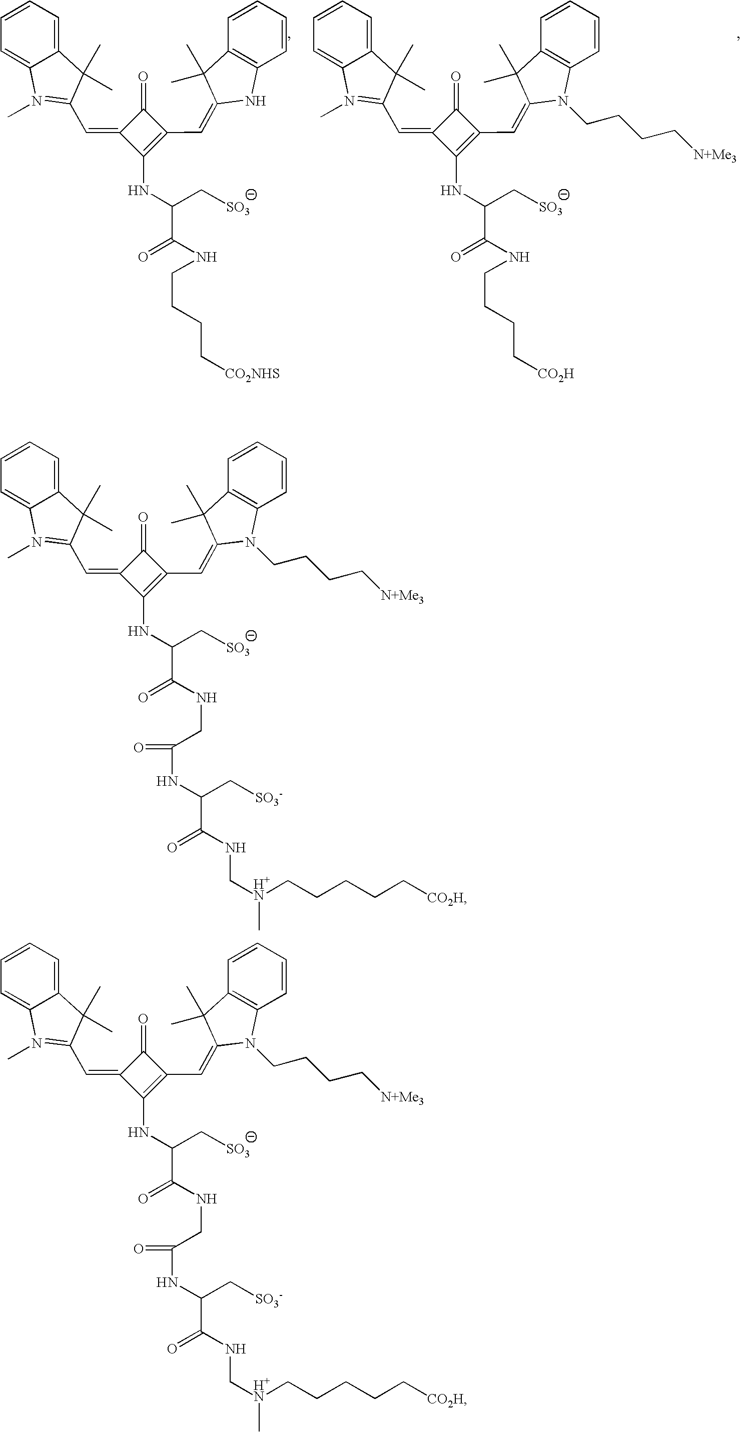

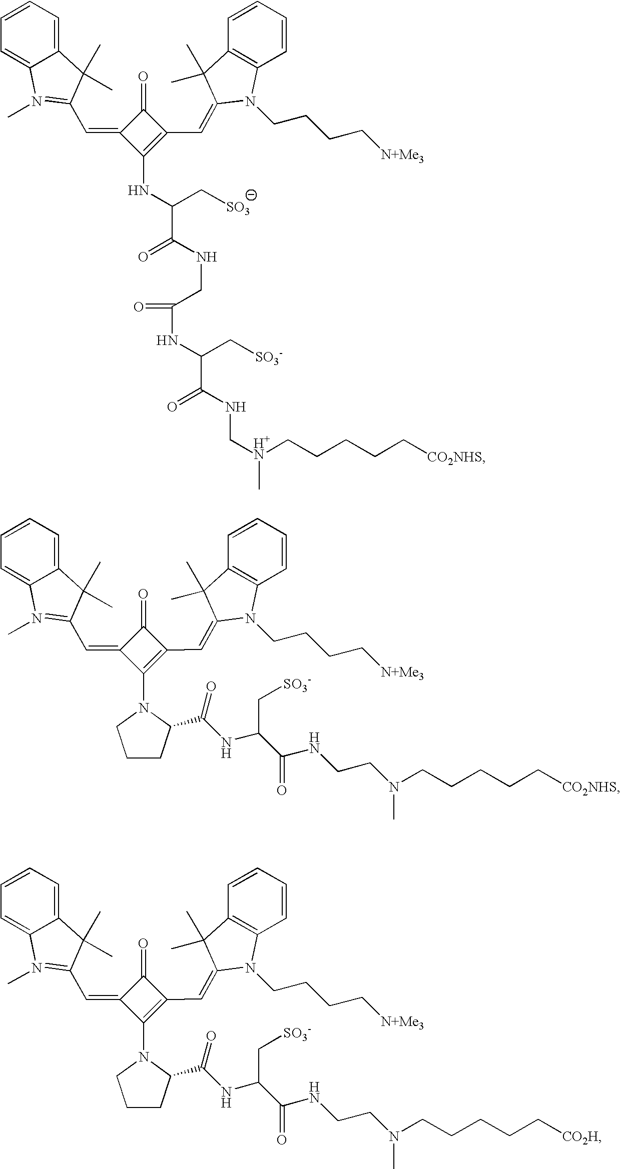

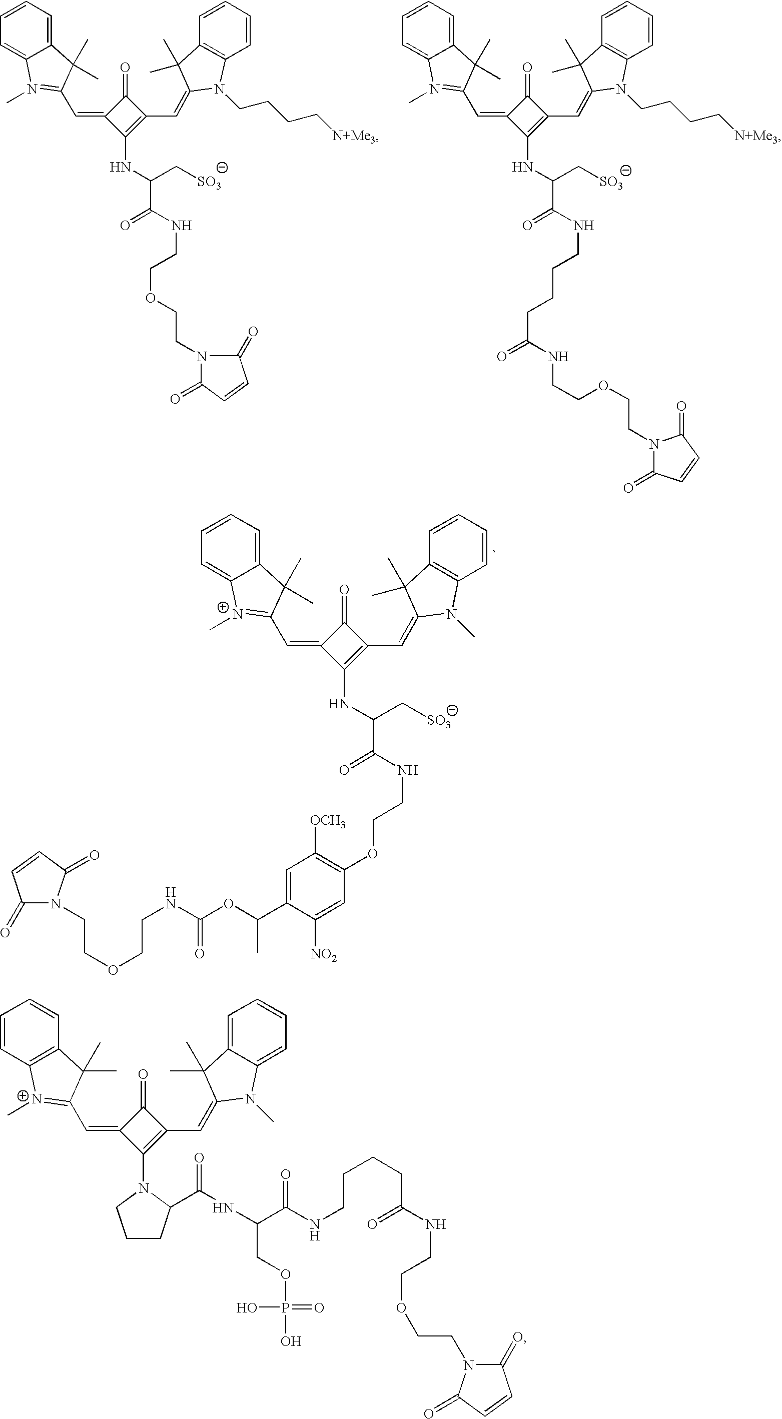

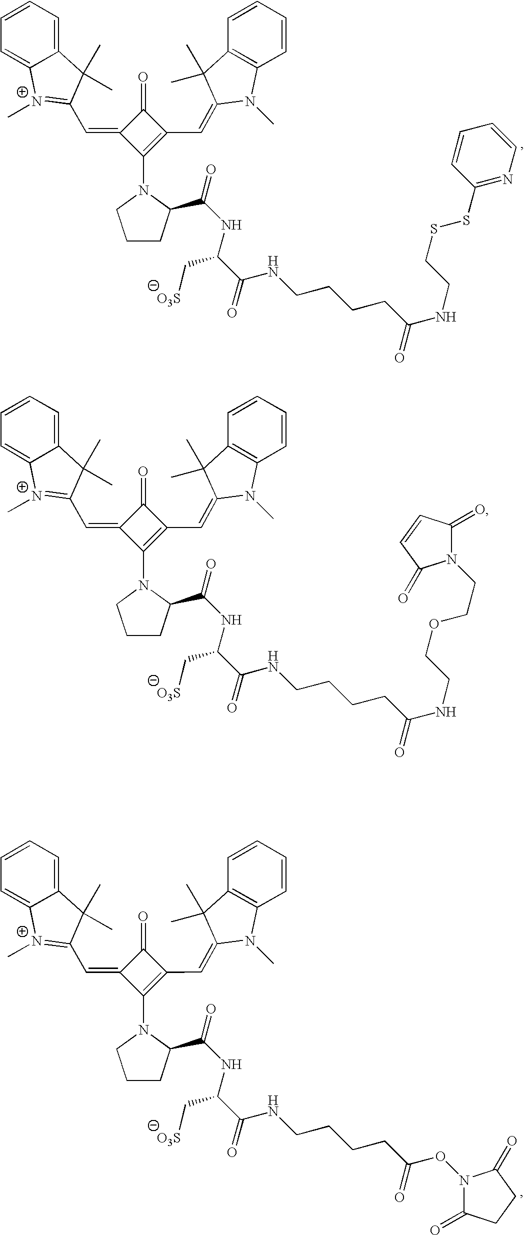

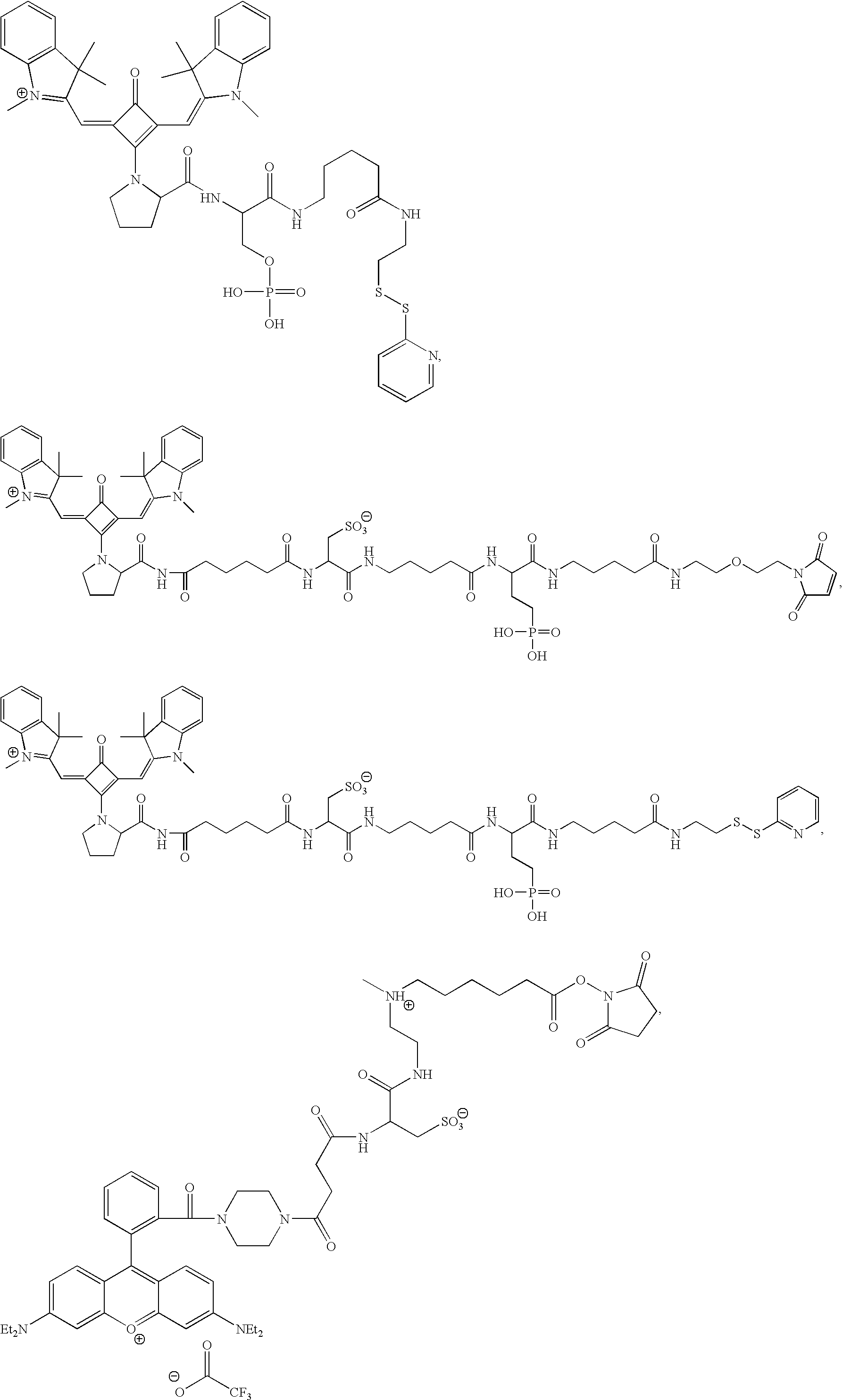

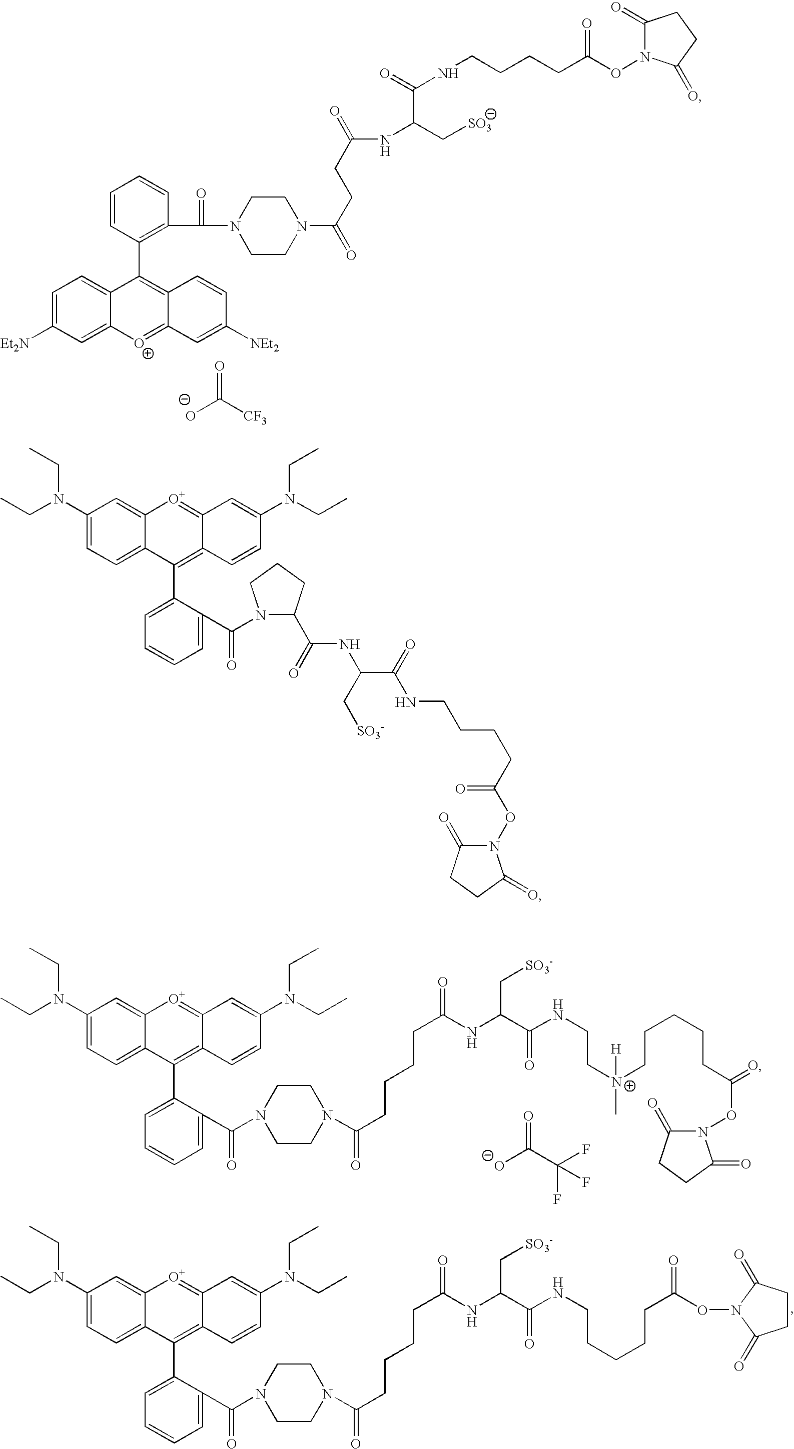

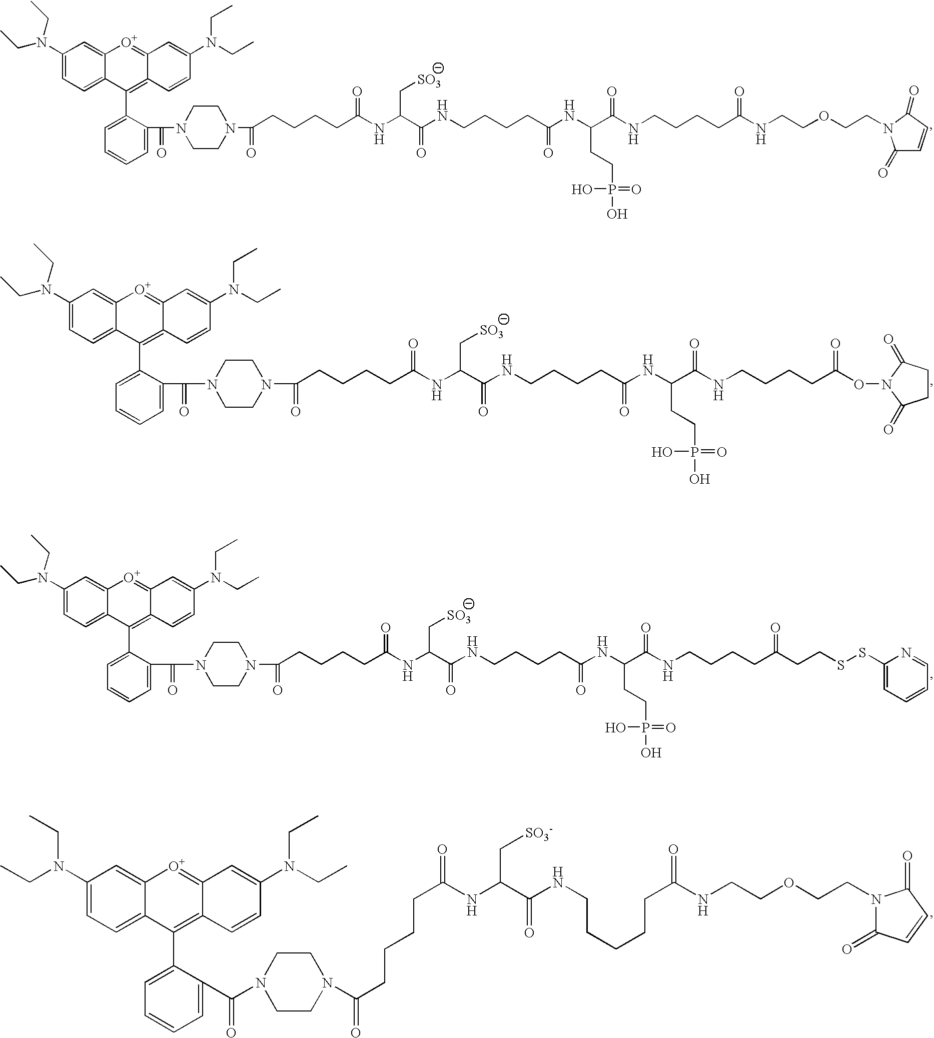

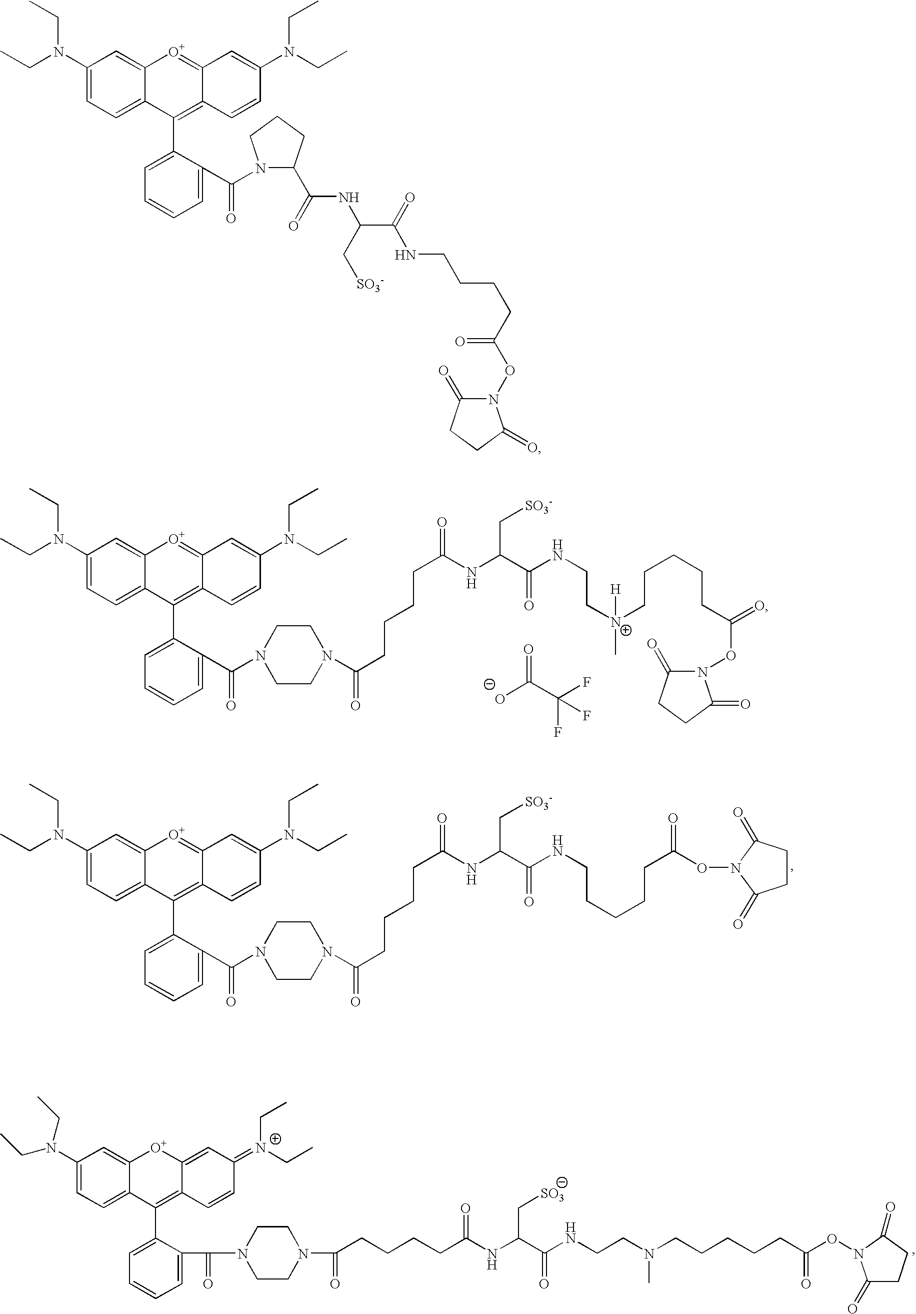

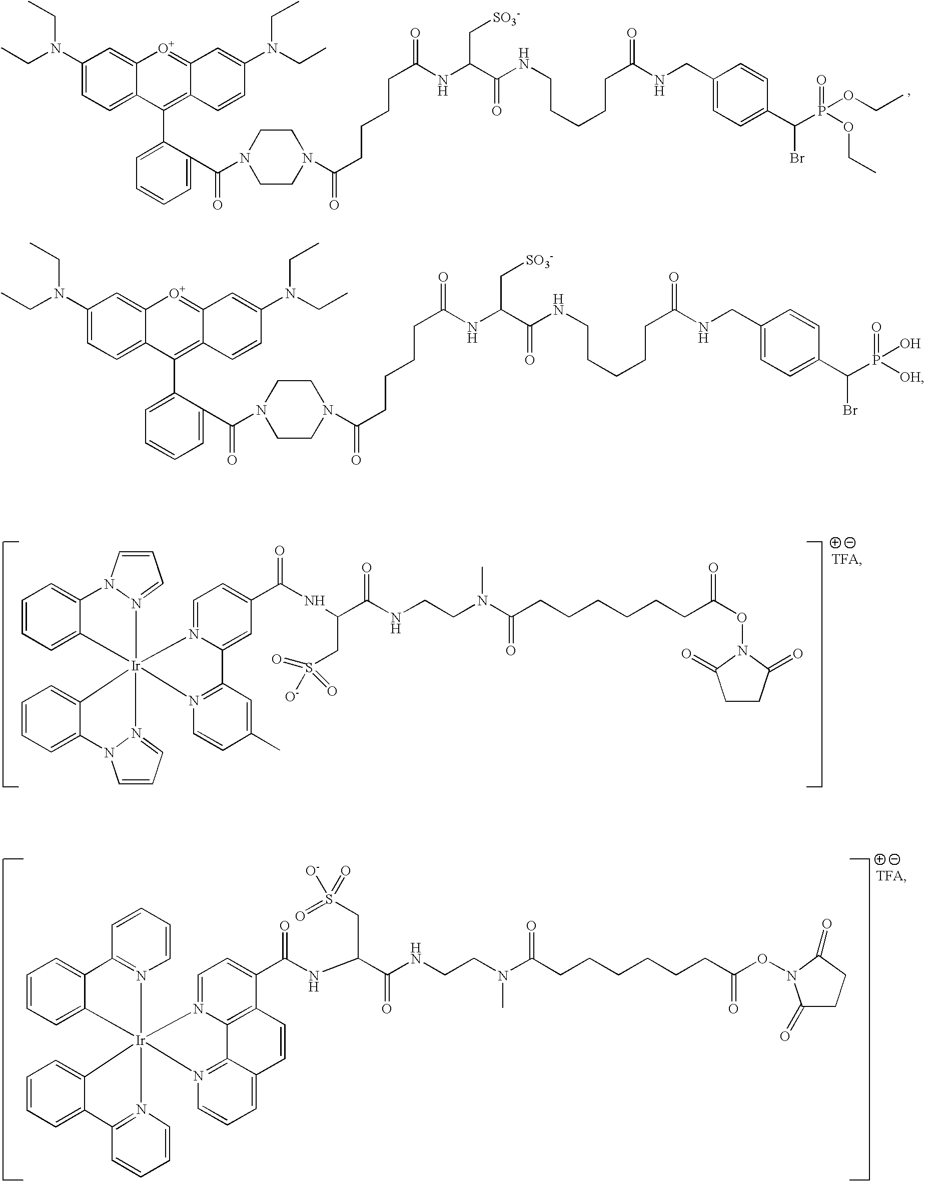

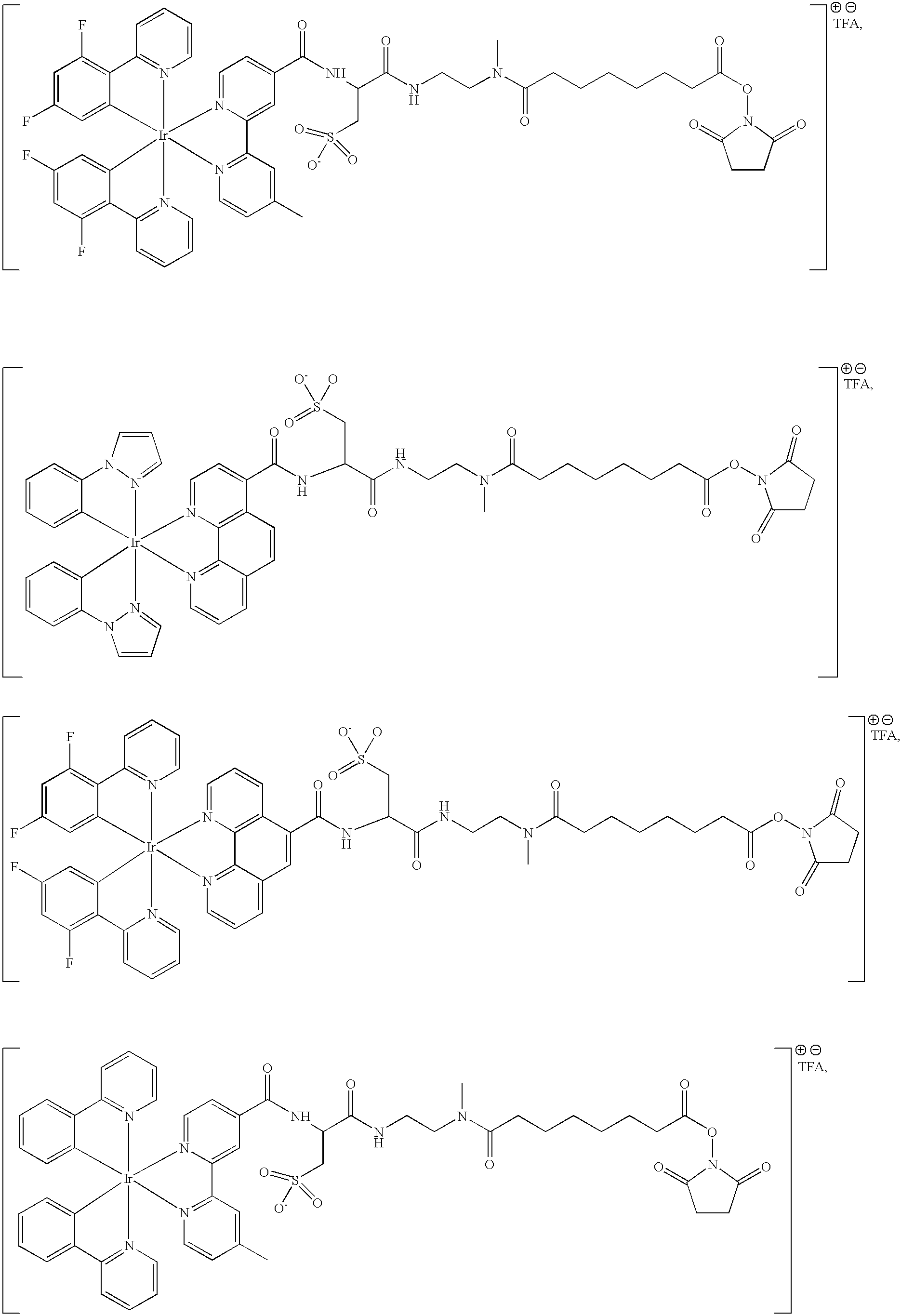

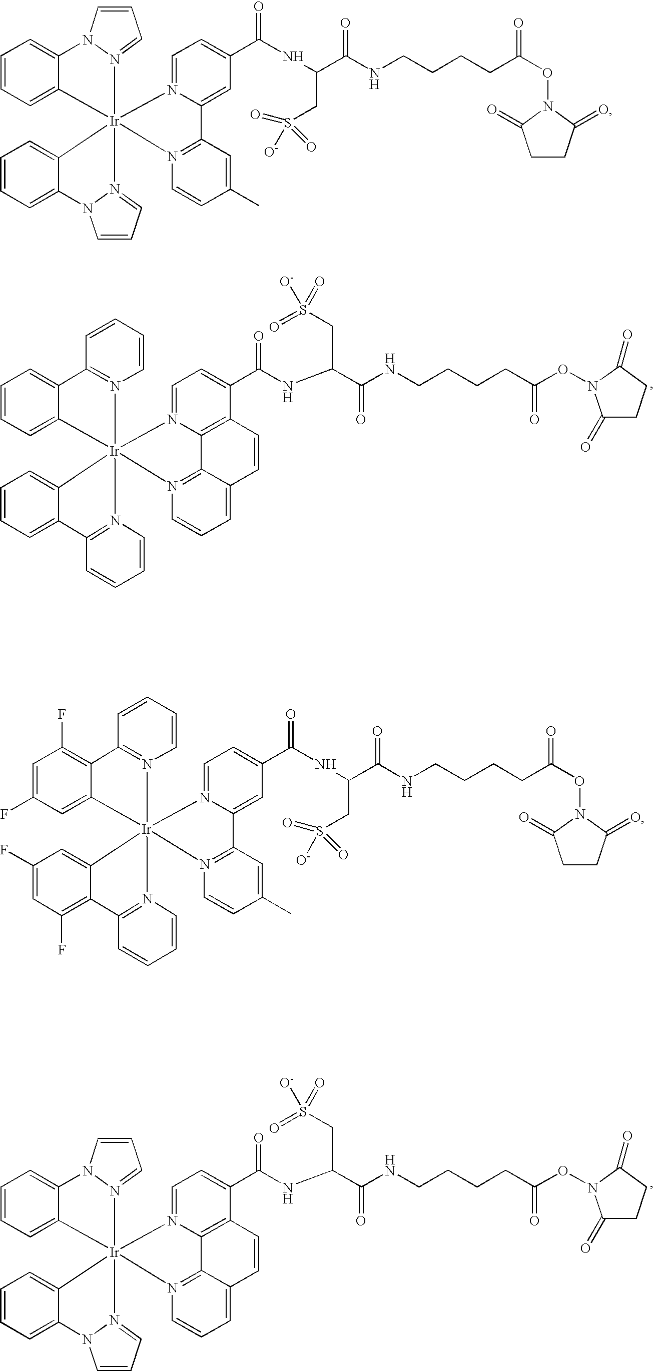

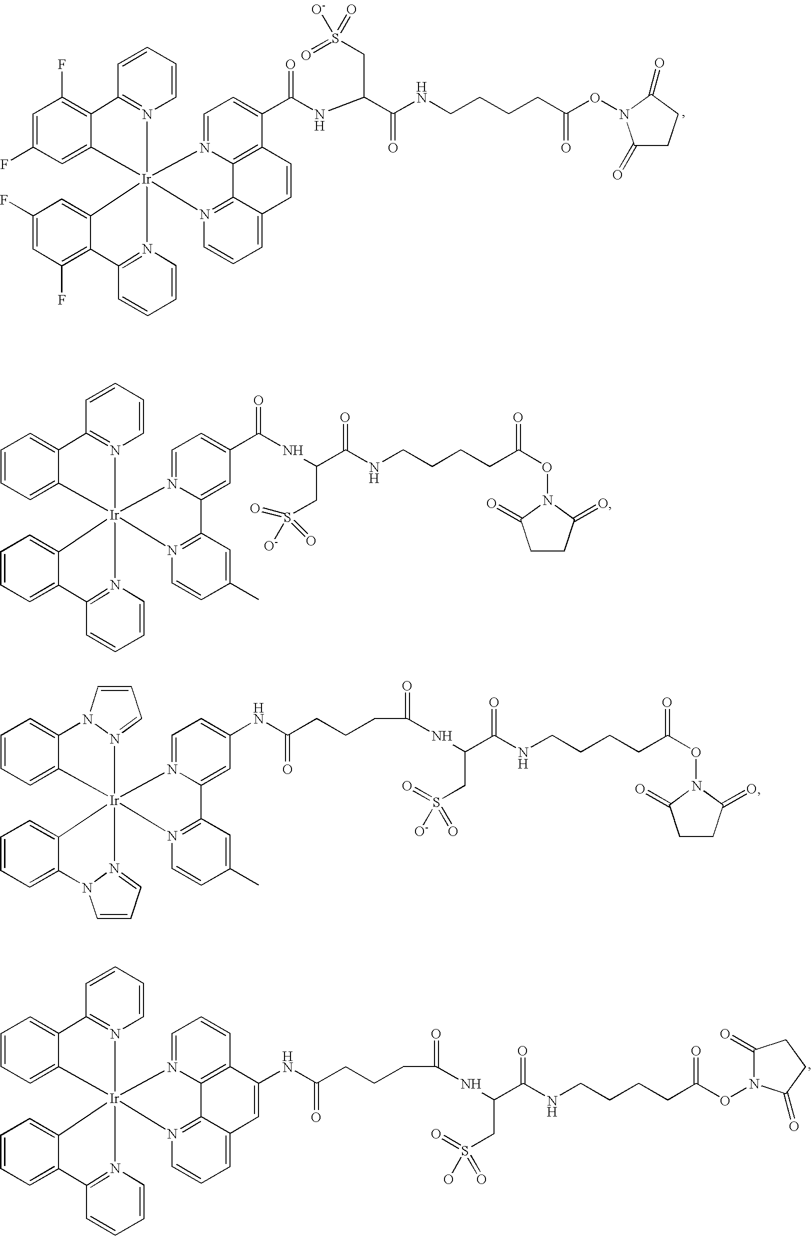

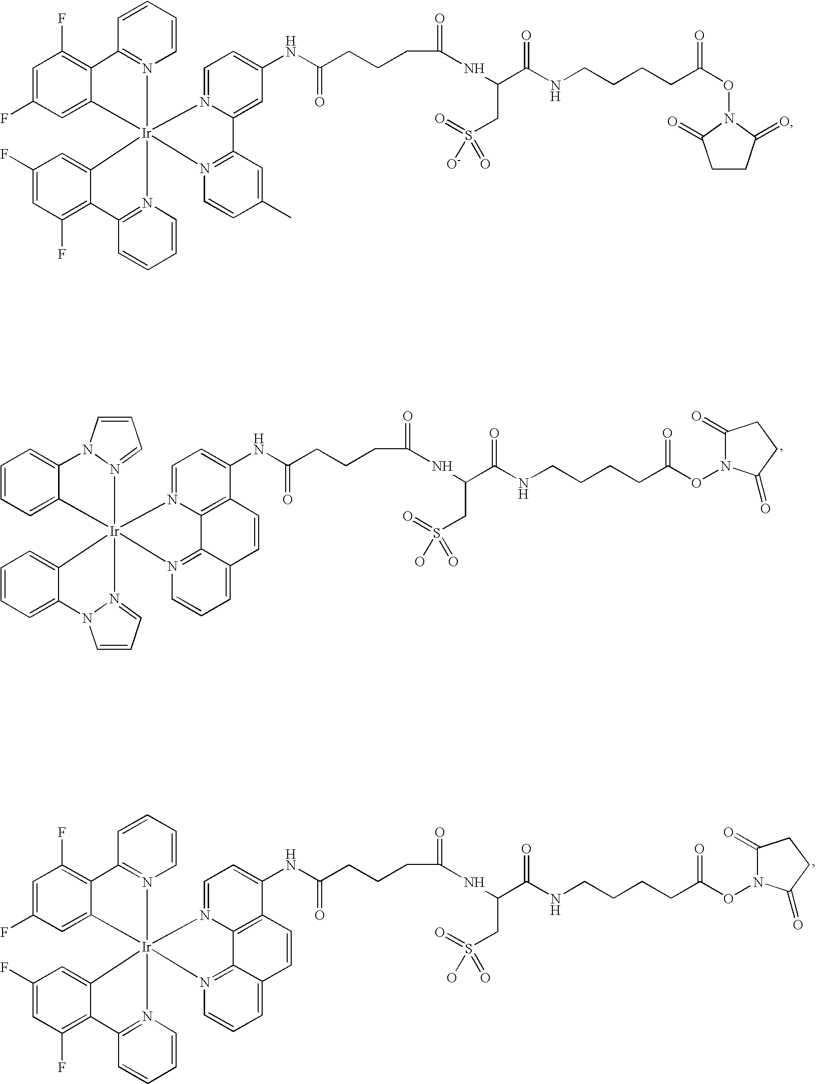

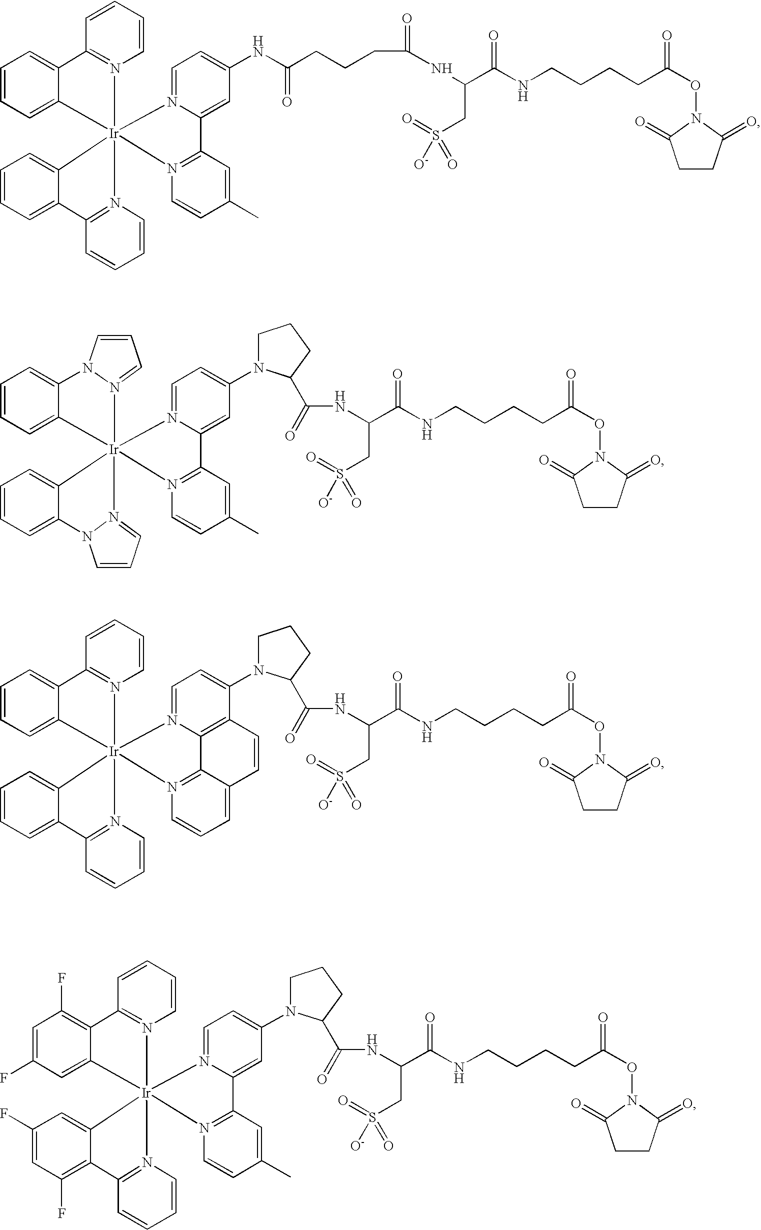

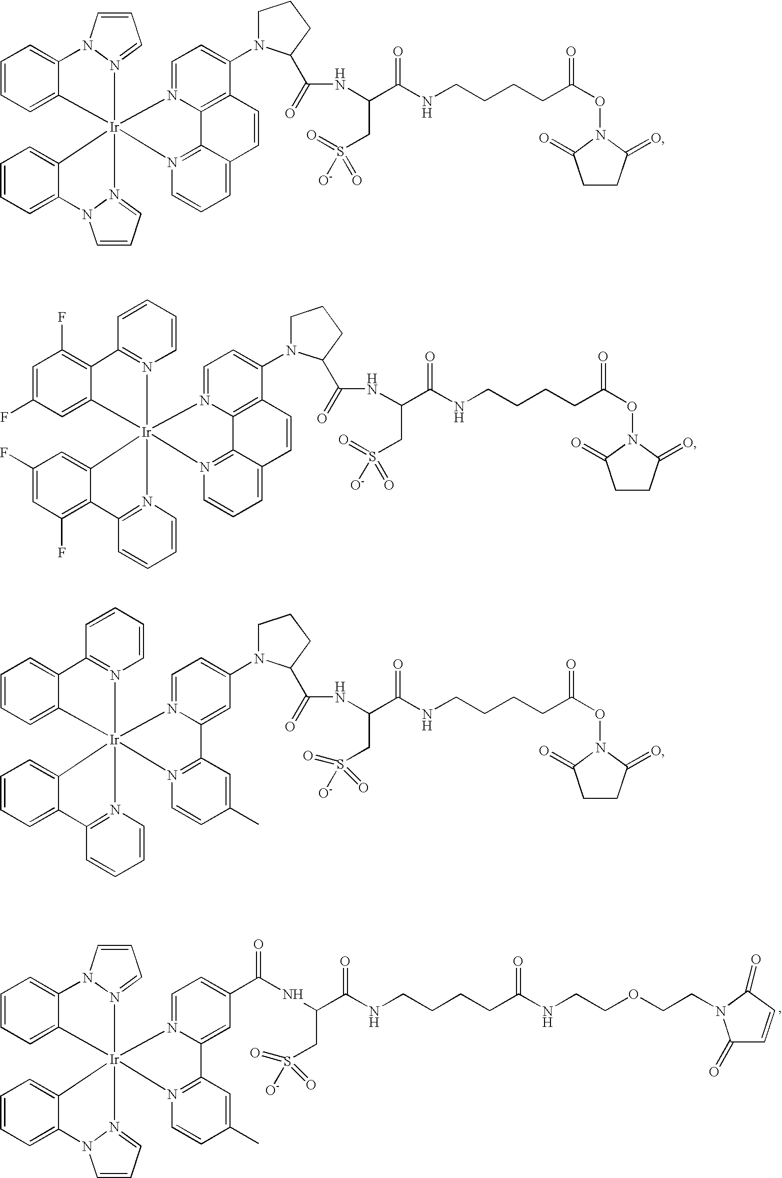

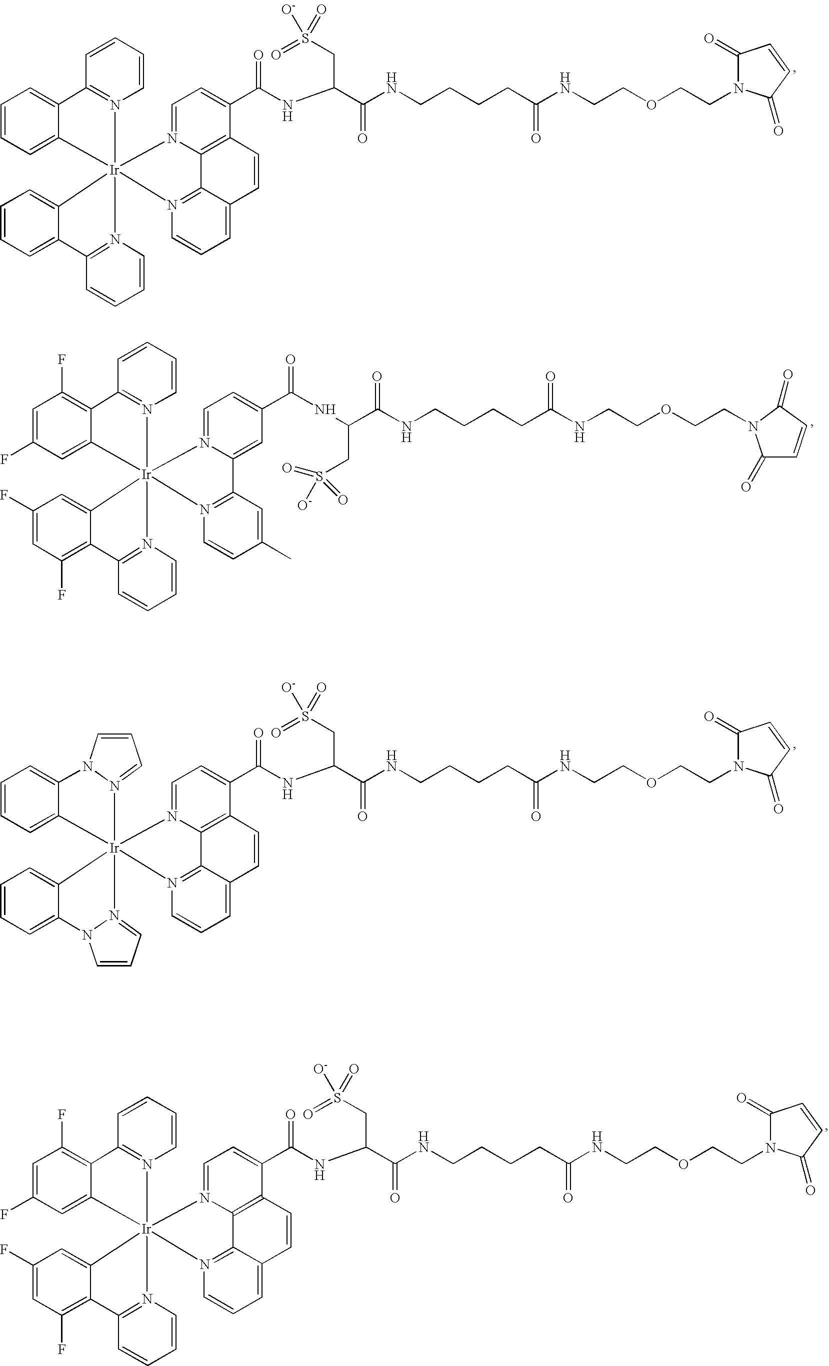

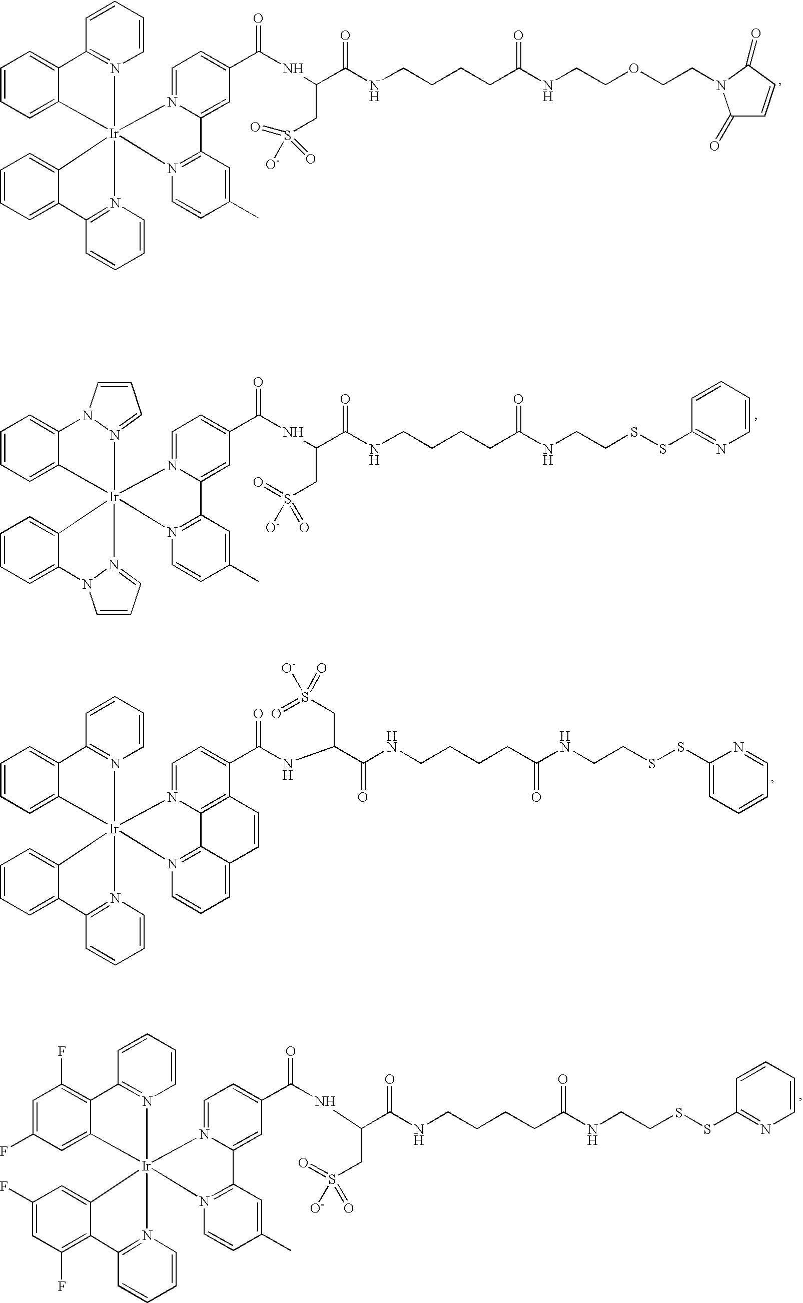

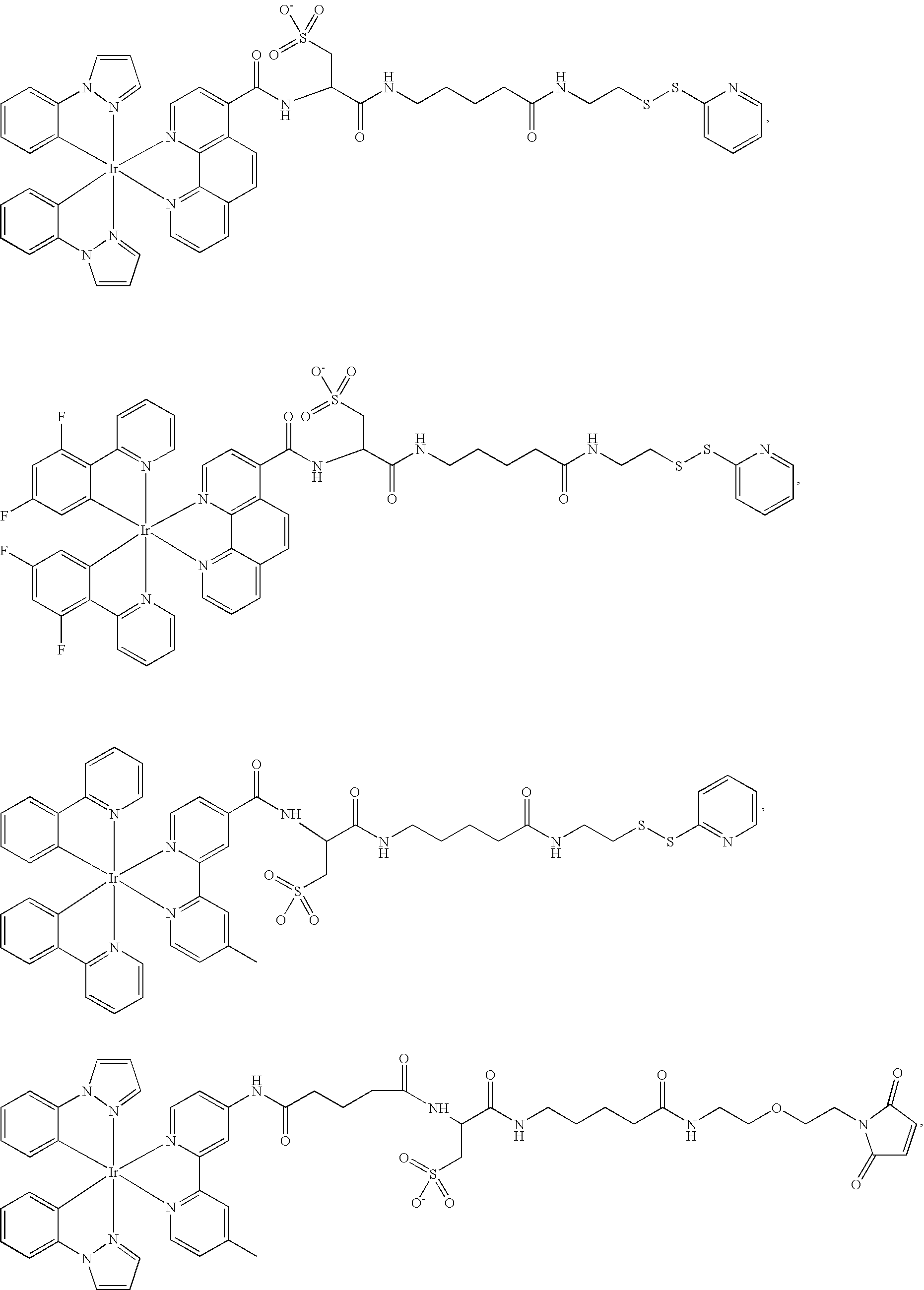

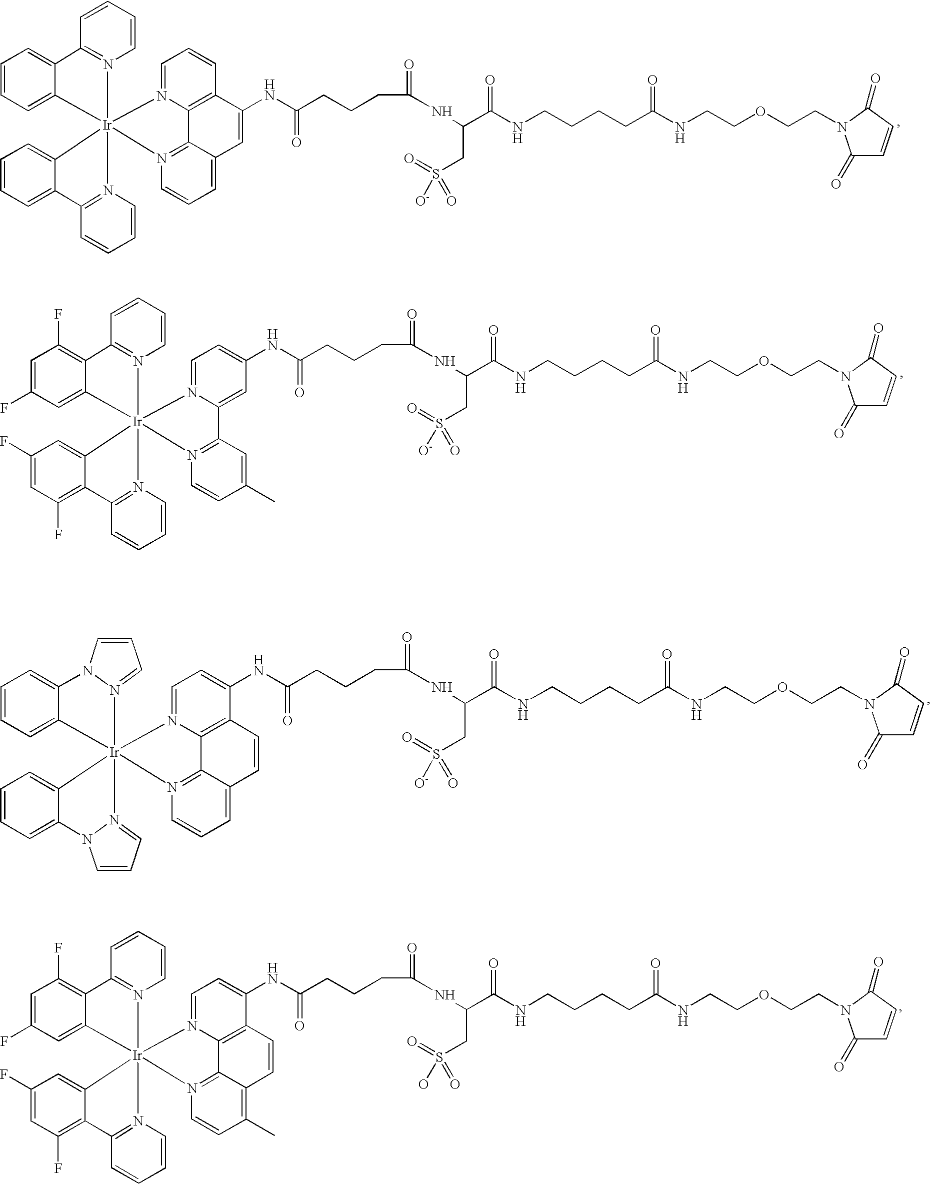

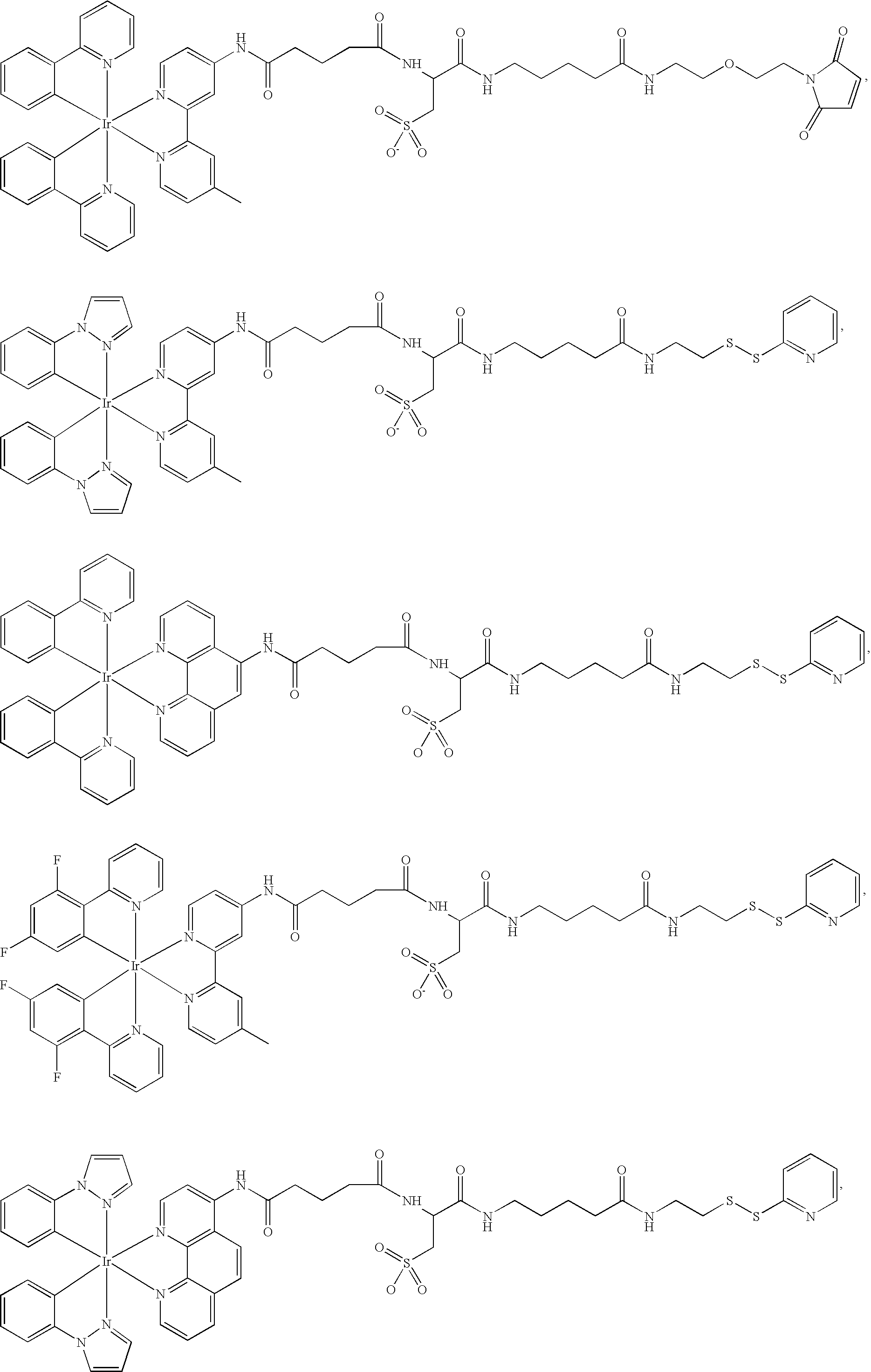

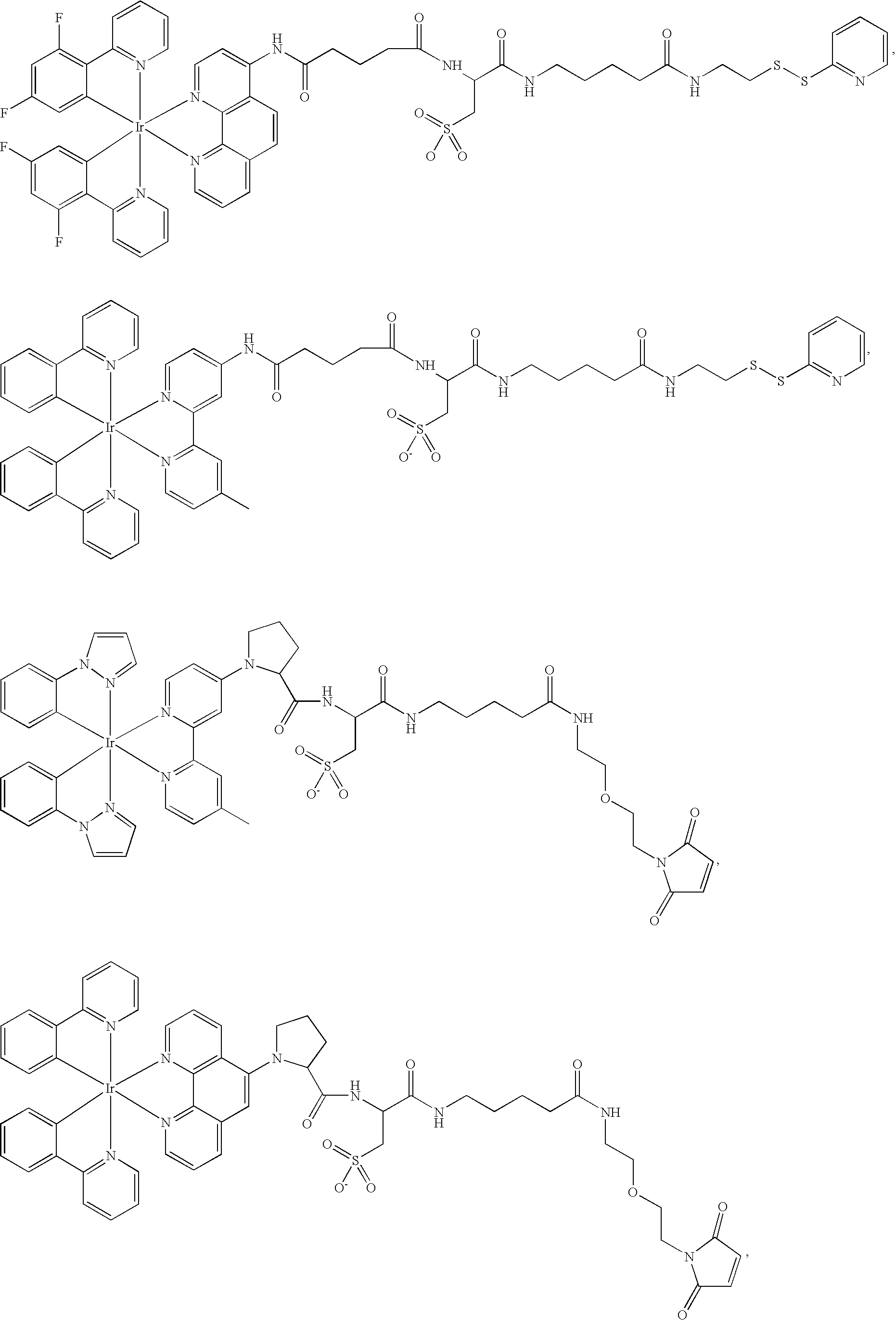

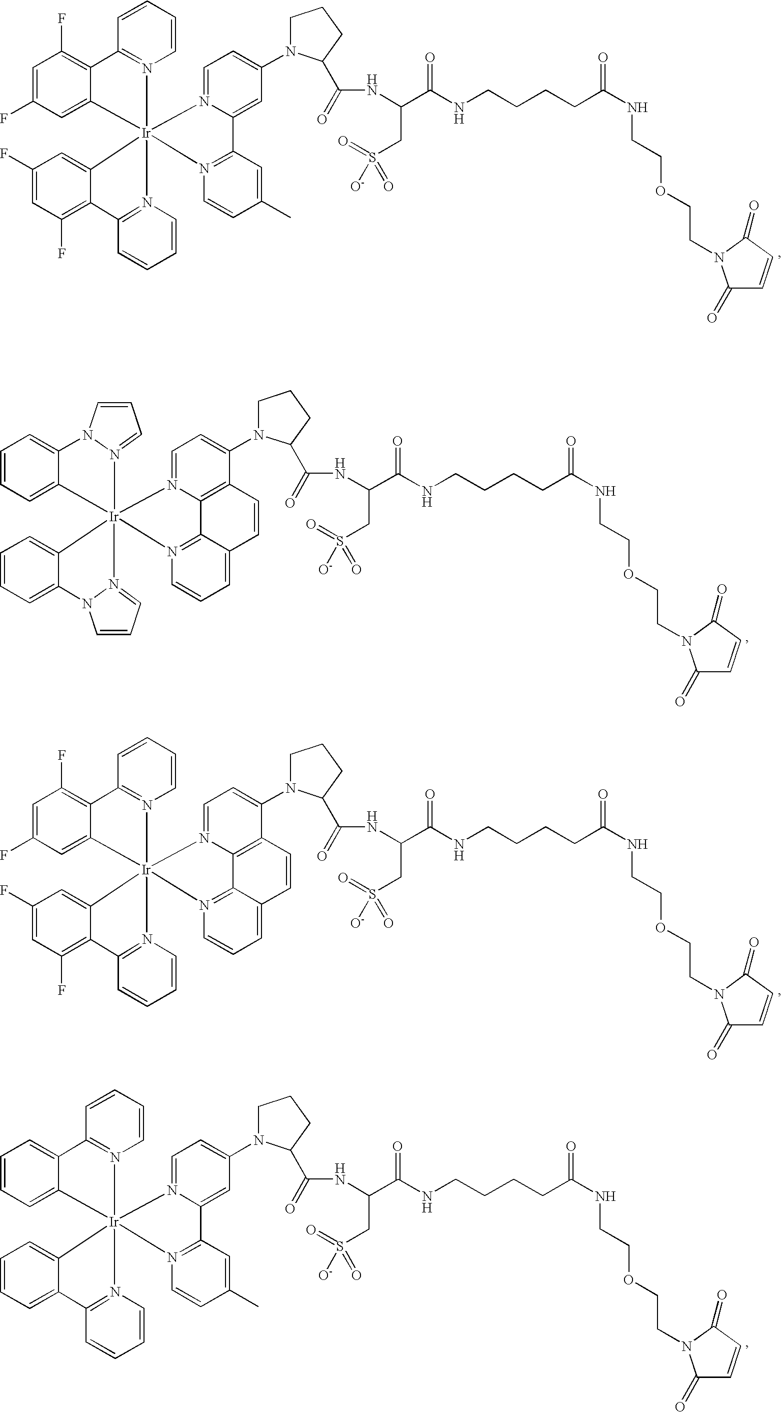

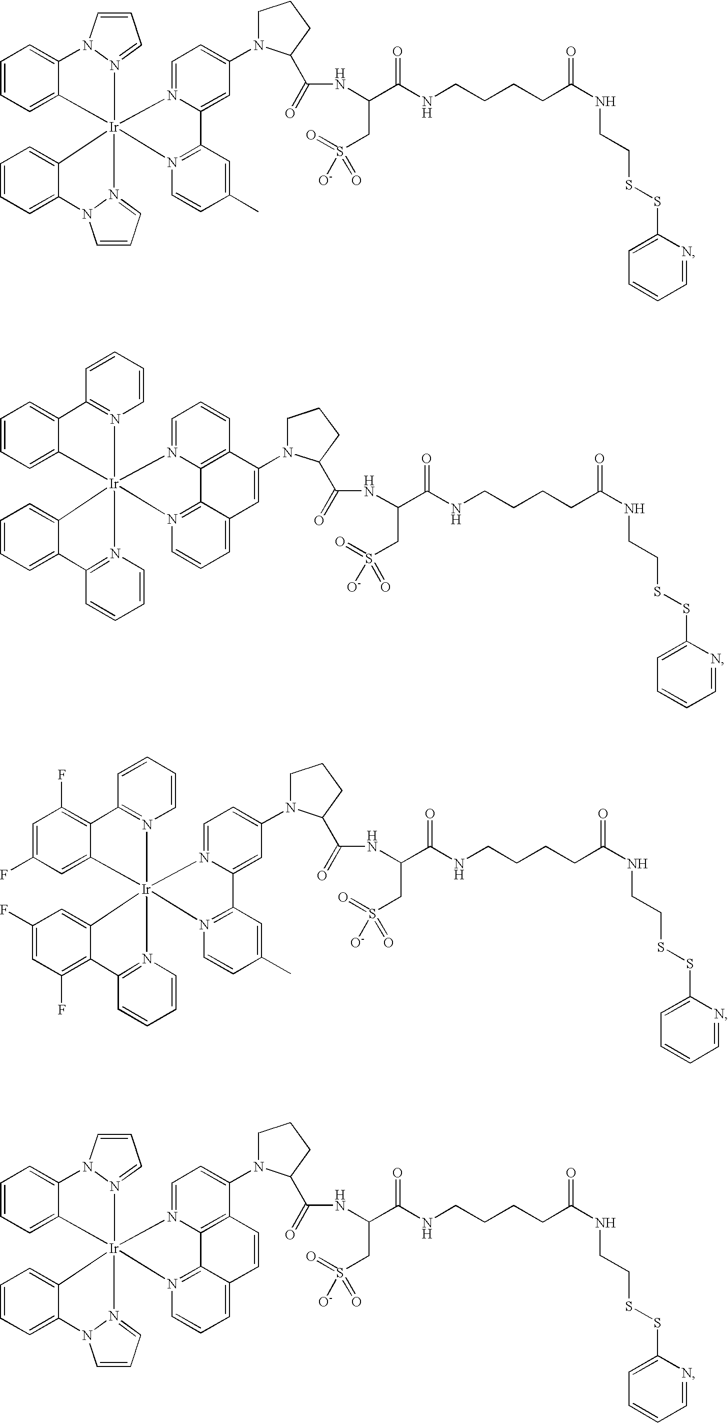

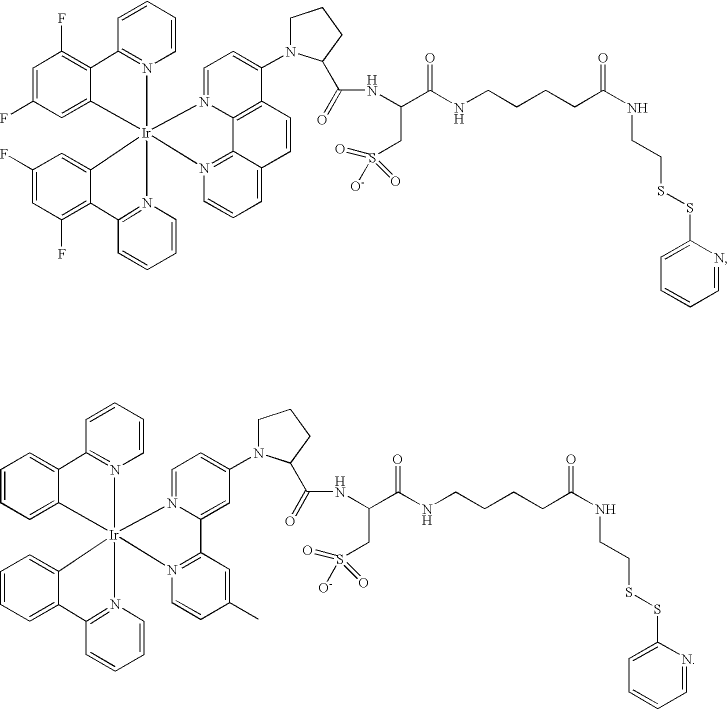

45. A set of at least two different optical labeling molecules according to claim 1 for use in labeling the proteins in at least two different target protein samples.

46. A method of differential analysis of proteins comprising:
providing at least two different samples of differently labeled proteins labeled with at least two different optical labeling molecules to form pluralities of differently labeled proteins, wherein said optical labeling molecule comprises a zwitterionic dye moiety, a linker, an optional titratable group that mimics the acid-base titration of the group labeled in the proteins, an optional cleavable group, an optional second label stable isotope group and an activator that covalently attaches the optical labeling molecule to the protein, mixing the different samples of differently labeled proteins together to form a mixture;
simultaneously separating the differently labeled proteins in the mixture to obtain a plurality of separated differently labeled proteins;
scanning the separated differently labeled proteins;
matching the same proteins from different samples that have been labeled with the different optical labeling molecules; and
simultaneously determining the changes in relative amounts of differently labeled proteins in the different samples by correlating said changes with the strength of the optical images of the labeled proteins.

47. A method according to claim 46, wherein said differently labeled proteins are produced by covalently labeling at least two different samples of proteins with at least two different optical labeling molecules to form pluralities of differently labeled proteins.

48. A method according to claim 46, wherein the differently labeled proteins are separated by a separation method selected from the group consisting of 1D gel electrophoresis, 2D gel electrophoresis, capillary electrophoresis, 1D chromatography, 2D chromatography, and 3D chromatography.

49. The method according to claim 46, wherein said optical labeling molecule is a fluorescent dye and a gel, obtained using 1D gel electrophoresis or 2D gel electrophoresis comprises the separated differently labeled proteins, is scanned with light excitation to provide fluorescent images of the differently colored optical labeling molecules.

50. The method according to claim 46, wherein the differential analysis of the proteins comprising at least one of the following analysis selected from the group consisting of relative amounts of protein, absolute amounts of protein, analysis of posttranslational modifications, analysis of enzyme activities, analysis of protein levels, analysis of cell or organelle surface-exposed proteins, phosphorylation states, nitrosylation states, and glycosidation states of proteins.

51. The method according to claim 49, wherein the matching step is performed to adjust the images to compensate for differences in gel mobility among proteins labeled with different optical labeling molecules using gel pattern matching software.

52. A method of differential analysis of proteins comprising:
covalently labeling at least two different samples of proteins with at least two different optical labeling molecules to form pluralities of differently labeled proteins, wherein said optical labeling molecule comprises an optical labeling molecule according to claim 1;
mixing the different samples of differently labeled proteins together to form a mixture;
simultaneously separating the differently labeled proteins in the mixture to obtain a plurality of separated differently labeled proteins;
scanning the separated differently labeled proteins;
matching the same proteins from different samples that have been labeled with the different optical labeling molecules; and
simultaneously determining the changes in relative amounts of differently labeled proteins in the different samples by correlating said changes with the strength of the optical images of the labeled proteins.

53. A method of differential analysis of proteins comprising:
covalently labeling at least two different samples of proteins with at least two different optical labeling molecules to form pluralities of differently labeled proteins, wherein said optical labeling molecule comprises an optical labeling molecule according to claim 44;
mixing the different samples of differently labeled proteins together to form a mixture;
simultaneously separating the differently labeled proteins in the mixture to obtain a plurality of separated differently labeled proteins;
scanning the separated differently labeled proteins;
matching the same proteins from different samples that have been labeled with the different optical labeling molecules; and
simultaneously determining the changes in relative amounts of differently labeled proteins in the different samples by correlating said changes with the strength of the optical images of the labeled proteins.

54. A method of labeling at least one target protein in a sample comprising:
covalently labeling at least one target protein with at least one optical labeling molecule according to claim 1.
